(19) United States
(12) Patent Application Publication
(10) Pub. No.: US 2024/0360476 A1
(43) Pub. Date: Oct. 31, 2024

(54) Genome Editing Compositions and Methods for Treatment of Myotonic Dystrophy

(71) Applicant: Prime Medicine, Inc., Cambridge, MA (US)

(72) Inventors: Tyler HARVEY, Cambridge, MA (US); Yu ZHENG, Cambridge, MA (US); Ekaterina Alexeevna KOSHELEVA, Cambridge, MA (US); Chicheng SUN, Cambridge, MA (US); Spencer TANG, Cambridge, MA (US)

(21) Appl. No.: 18/294,729

(22) PCT Filed: Aug. 5, 2022
(86) PCT No.: PCT/US2022/039644

§ 371 Date: Feb. 2, 2024

Related U.S. Application Data
(60) Provisional application No. 63/229,768, filed on Aug. 5, 2021.

(51) Int. Cl. C12N 15/90; C12N 9/12; C12N 9/22; C12N 15/11

(52) U.S. Cl. CPC C12N 15/907; C12N 9/1276; C12N 9/22; C12N 15/11; C12Y 207/07049

(57) ABSTRACT

Provided herein are prime editing methods and compositions for treatment of genetic disorders such as myotonic dystrophy.

### CROSS REFERENCE TO RELATED APPLICATIONS

The present application claims priority to and the benefit of U.S. Provisional Application No. 63/229,768, filed Aug. 5, 2021, the contents of which are herein incorporated by reference in their entirety.

### BACKGROUND

The present invention describes dual prime editing as a genome editing approach for treating genetic diseases, for example, repeat expansion disorder myotonic dystrophy type 1 (DM1).

DM1 is an autosomal dominant neuromuscular disorder. Affected individuals display a wide range of symptoms including myotonia, skeletal muscle weakness and wasting, cardiac conduction abnormalities, and cataracts.

DM1 is associated with mutations in the DMPK gene. The DMPK gene encodes the myotonic dystrophy protein kinase (DMPK protein).

The 3′ untranslated region of the DMPK gene contains multiple copies of a CTG trinucleotide repeat. In most healthy individuals, the number of CTG repeats in the DMPK gene ranges from 5 to 34. Expansion of the CTG repeat numbers in the DMPK gene above a normal range results in increased number of repeats in the DMPK transcript, which can form stable double-stranded structures that have strong affinity for other functional proteins, e.g., proteins of the Muscleblind-like (MBNL) family, and prohibit the proteins from performing their normal functions. The expanded repeats can also result in abnormal splicing patterns in adult tissues, which can affect expression of hundreds of genes.

The severity of DM1 symptoms is correlated with the number of abnormally expanded CTG repeats in the DMPK gene. People with symptoms of DM1, including muscle weakness and wasting beginning in adulthood, usually have above 100 to 1000 CTG repeats. People born with more severe, congenital form of DM1 tend to have more than 1,000 CTG repeats in their cells. People with mild form of DM1 symptoms usually have between 50 and 150 CTG repeats in their cells. While smaller number of CTG repeats may only relate to mild symptoms or may be asymptomatic, as the altered DMPK gene is passed from one generation to the next, the size of the CTG repeat expansion may increases in size. For example, people with 35 to 49 CTG repeats do not develop DM symptoms, but their children may be at risk of having the disorder if the number of CTG repeats increases.

Current developments in treatment for DM1 include small molecules such as mexiletine and metformin and oligonucleotide based therapies such as siRNA and antisense nucleotides (ASOs).

This disclosure provides dual prime editing methods and compositions for editing the DMPK gene and removing pathogenic trinucleotide repeats associated with DM1.

### SUMMARY OF THE INVENTION

The prime editing systems described herein comprise compositions, systems, and methods that relate to programmable editing of a double stranded target DNA, e.g., a target gene such as a DMPK gene, using two or more prime editing guide RNAs (PEgRNAs) each complexed with a prime editor (“dual prime editing”). The compositions, systems, and methods described herein may be used to incorporate one or more intended nucleotide edits into the double stranded target DNA. In some embodiments, compositions, systems and methods relating to dual prime editing may be used to make alterations in a target sequence of a target gene, for example, a DMPK gene.

In some embodiments, dual prime editing incorporates one or more intended nucleotide edits into the target DNA through excision of an endogenous DNA segment and/or replacement of the endogenous DNA segment with newly synthesized DNA via target-primed DNA synthesis. Dual prime editing involves two different PEgRNAs each complexed with a prime editor. Without wishing to be bound by any particular theory, each of the two PEgRNAs comprises a region of complementarity to a distinct search target sequence of a target DNA, for example a DMPK gene, wherein the two distinct search target sequences are on the two complementary strands of the target DNA. In some embodiments, the two PEgRNAs each can direct a prime editor to initiate the prime editing process on the two complementary strands of the target DNA, thereby incorporating one or more intended nucleotide edits into the target DNA, e.g., the DMPK gene.

In one aspect, a prime editing composition of the present disclosure comprises: (A) a first prime editing guide RNA (PEgRNA) or one or more polynucleotides encoding the first PEgRNA, and (B) a second PEgRNA or one or more polynucleotides encoding the second PEgRNA; wherein the first PEgRNA comprises: (i) a first spacer that is complementary to a first search target sequence on a first strand of a DMPK gene; (ii) a first gRNA core capable of binding to a Cas9 protein; and (iii) a first extension arm comprising a first editing template and a first primer binding site (PBS); wherein the first spacer comprises at its 3′ end nucleotides 5-20 of a sequence selected from the group consisting of SEQ ID NOs: 1, 20, 39, 58, 77, 484, 518, 536, 554, 572, 590, 912, 929, 947, 965, 983, 1001, 1019, and 1341, and wherein the first PBS comprises at its 5′ end a sequence that is the reverse complement of nucleotides 15-17 of the selected sequence; wherein the second PEgRNA comprises: (i) a second spacer that is complementary to a second search target sequence on a second strand of the DMPK gene complementary to the first strand; (ii) a second gRNA core capable of binding to a Cas9 protein; and (iii) a second extension arm comprising a second editing template and a second PBS; wherein the second spacer comprises at its 3′ end nucleotides 5-20 of a sequence selected from the group consisting of SEQ ID NOs: 1359, 1381, 1403, 1503, 1525, 1864, 1882, 1900, 1918, 1936, 2263, 2281, 2299, 2317, 2335, 2353, 2673, and wherein the second PBS comprises at its 5′ end a sequence that is the reverse complement of nucleotides 15-17 of the selected sequence; and wherein (a) the first editing template comprises a region of complementarity to the second editing template; (b) the first editing template comprises nucleotides 8-17 of the selected sequence for the second spacer, and the second editing template comprises nucleotides 8-17 of the selected sequence for the first spacer; or (c) the first editing template comprises nucleotides 8-17 of the selected sequence for the second spacer, and a region of complementarity to the second editing template, and the second editing template comprises nucleotides 8-17 of the selected sequence for the first spacer, and a region of complementarity to the first editing template.

In some embodiments, the selected sequence for the first spacer is SEQ ID NOs: 77, 484, 536, 590, or 1019.

In some embodiments, the selected sequence for the second spacer is SEQ ID NO: 1525, 1900, 1936, 2263, 2281, 2299, 2353, or 2673.

In some embodiments, the selected sequence for the first spacer is SEQ ID NO: 77, 536, or 1019.

In some embodiments, the selected sequence for the second spacer is SEQ ID NO: 1900, 1936, or 2673.

In some embodiments, the first spacer and/or the second spacer is from 16 to 22 nucleotides in length.

In some embodiments, the first spacer and/or the second spacer comprises at its 3′ the selected sequence as described in any of the embodiments above.

In some embodiments, the first spacer and/or the second spacer is 20 nucleotides in length and comprises the selected sequence.

In some embodiments, the first gRNA core and the second gRNA core comprise the same sequence.

In some embodiments, the first gRNA core and the second gRNA core each comprises SEQ ID NO: 3641.

In some embodiments, the first spacer, the first gRNA core, the first editing template, and the first PBS form a contiguous sequence in a single molecule.

In some embodiments, the first PEgRNA comprises from 5′ to 3′ the first spacer, the first gRNA core, the first editing template, and the first PBS.

In some embodiments, the second spacer, the second gRNA core, the second editing template, and the second PBS form a contiguous sequence in a single molecule.

In some embodiments, the second pegRNA comprises from 5′ to 3′ the second spacer, the second gRNA core, the second editing template, and the second PBS.

In some embodiments, the first PBS is at least 8 nucleotides in length and comprises at its 5′ end a sequence that is the reverse complement of nucleotides 10-17 of the selected sequence for the first spacer.

In some embodiments, the first PBS is 8-17 nucleotides in length and comprises at its 5′ end a sequence that is the reverse complement of nucleotides 10-17, 9-17, 8-17, 7-17, 6-17, 5-17, 4-17, 3-17, 2-17, or 1-17 of the selected sequence for the first spacer. In some embodiments, the first PBS is 8-14 nucleotides in length. In some embodiments, the first PBS is 8-12 nucleotides in length. In some embodiments, the first PBS is 8-10 nucleotides in length. In some embodiments, the first PBS is 8-9 nucleotides in length. In some embodiments, the first PBS is 10 nucleotides in length. In some embodiments, the second PBS is at least 8 nucleotides in length and comprises at its 5′ end a sequence that is the reverse complement of nucleotides 10-17 of the selected sequence for the second spacer.

In some embodiments, the second PBS is 8-17 nucleotides in length and comprises at its 5′ end a sequence that is the reverse complement of nucleotides 10-17, 9-17, 8-17, 7-17, 6-17, 5-17, 4-17, 3-17, 2-17, or 1-17 of the selected sequence for the second spacer. In some embodiments, the second PBS is 8-14 nucleotides in length. In some embodiments, the second PBS is 8-12 nucleotides in length. In some embodiments, the second PBS is 8-10 nucleotides in length. In some embodiments, the second PBS is 8-9 nucleotides in length. In some embodiments, wherein the second PBS is 10 nucleotides in length.

In some embodiments, the prime editing composition comprises the first PEgRNA and the second PEgRNA as described in any of the embodiments above, wherein the first editing template comprises a region of complementarity to the second editing template. In some embodiments, the region of complementarity is about 20 to about 80 nucleotides in length. In some embodiments, the region of complementarity is about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, or about 80 nucleotides in length. In some embodiments, the region of complementarity is between 20 and 80 nucleotides; between 21 and 80 nucleotides; between 22 and 80 nucleotides; between 23 and 80 nucleotides; between 24 and 80 nucleotides; between 25 and 80 nucleotides; between 26 and 80 nucleotides; between 27 and 80 nucleotides; between 28 and 80 nucleotides; between 29 and 80 nucleotides; between 30 and 80 nucleotides; between 31 and 80 nucleotides; between 32 and 80 nucleotides; between 33 and 80 nucleotides; between 34 and 80 nucleotides; between 35 and 80 nucleotides; between 36 and 80 nucleotides; between 37 and 80 nucleotides; between 38 and 80 nucleotides; between 39 and 80 nucleotides; between 40 and 80 nucleotides; between 41 and 80 nucleotides; between 42 and 80 nucleotides; between 43 and 80 nucleotides; between 44 and 80 nucleotides; between 45 and 80 nucleotides; between 46 and 80 nucleotides; between 47 and 80 nucleotides; between 48 and 80 nucleotides; between 49 and 80 nucleotides; between 50 and 80 nucleotides; between 51 and 80 nucleotides; between 52 and 80 nucleotides; between 53 and 80 nucleotides; between 54 and 80 nucleotides; between 55 and 80 nucleotides; between 56 and 80 nucleotides; between 57 and 80 nucleotides; between 58 and 80 nucleotides; between 59 and 80 nucleotides; between 60 and 80 nucleotides; between 61 and 80 nucleotides; between 62 and 80 nucleotides; between 63 and 80 nucleotides; between 64 and 80 nucleotides; between 65 and 80 nucleotides; between 66 and 80 nucleotides; between 67 and 80 nucleotides; between 68 and 80 nucleotides; between 69 and 80 nucleotides; between 70 and 80 nucleotides; between 71 and 80 nucleotides; between 72 and 80 nucleotides; between 73 and 80 nucleotides; between 74 and 80 nucleotides; between 75 and 80 nucleotides; between 76 and 80 nucleotides; between 77 and 80 nucleotides; between 78 and 80 nucleotides; between 20 and 80 nucleotides; between 20 and 79 nucleotides; between 20 and 78 nucleotides; between 20 and 77 nucleotides; between 20 and 76 nucleotides; between 20 and 75 nucleotides; between 20 and 74 nucleotides; between 20 and 73 nucleotides; between 20 and 72 nucleotides; between 20 and 71 nucleotides; between 20 and 70 nucleotides; between 20 and 69 nucleotides; between 20 and 68 nucleotides; between 20 and 67 nucleotides; between 20 and 66 nucleotides; between 20 and 65 nucleotides; between 20 and 64 nucleotides; between 20 and 63 nucleotides; between 20 and 62 nucleotides; between 20 and 61 nucleotides; between 20 and 60 nucleotides; between 20 and 59 nucleotides; between 20 and 58 nucleotides; between 20 and 57 nucleotides; between 20 and 56 nucleotides; between 20 and 55 nucleotides; between 20 and 54 nucleotides; between 20 and 53 nucleotides; between 20 and 52 nucleotides; between 20 and 51 nucleotides; between 20 and 50 nucleotides; between 20 and 49 nucleotides; between 20 and 48 nucleotides; between 20 and 47 nucleotides; between 20 and 46 nucleotides; between 20 and 45 nucleotides; between 20 and 44 nucleotides; between 20 and 43 nucleotides; between 20 and 42 nucleotides; between 20 and 41 nucleotides; between 20 and 40 nucleotides; between 20 and 39 nucleotides; between 20 and 38 nucleotides; between 20 and 37 nucleotides; between 20 and 36 nucleotides; between 20 and 35 nucleotides; between 20 and 34 nucleotides; between 20 and 33 nucleotides; between 20 and 32 nucleotides; between 20 and 31 nucleotides; between 20 and 30 nucleotides; between 20 and 29 nucleotides; between 20 and 28 nucleotides; between 20 and 27 nucleotides; between 20 and 26 nucleotides; between 20 and 25 nucleotides; between 20 and 24 nucleotides; between 20 and 23 nucleotides; or between 20 and 22 nucleotides in length. In some embodiments, the region of complementarity is at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, or at least 80 nucleotides in length. In some embodiments, the region of complementarity that is no more than 20, no more than 21, no more than 22, no more than 23, no more than 24, no more than 25, no more than 26, no more than 27, no more than 28, no more than 29, no more than 30, no more than 31, no more than 32, no more than 33, no more than 34, no more than 35, no more than 36, no more than 37, no more than 38, no more than 39, no more than 40, no more than 41, no more than 42, no more than 43, no more than 44, no more than 45, no more than 46, no more than 47, no more than 48, no more than 49, no more than 50, no more than 51, no more than 52, no more than 53, no more than 54, no more than 55, no more than 56, no more than 57, no more than 58, no more than 59, no more than 60, no more than 61, no more than 62, no more than 63, no more than 64, no more than 65, no more than 66, no more than 67, no more than 68, no more than 69, no more than 70, no more than 71, no more than 72, no more than 73, no more than 74, no more than 75, no more than 76, no more than 77, no more than 78, no more than 79, or no more than 80 nucleotides in length.

In some embodiments, the region of complementarity is about 20 to about 40 nucleotides in length.

In some embodiments, the region of complementarity is 23-38 nucleotides in length.

In some embodiments, the region of complementarity is about 10-19 nucleotides in length.

In some embodiments, the region of complementarity is 10-19 nucleotides in length.

In some embodiments, the region of complementarity is about 20-29 nucleotides in length.

In some embodiments, the region of complementarity is 20-29 nucleotides in length.

In some embodiments, the region of complementarity is about 30-40 nucleotides in length.

In some embodiments, the region of complementarity is 30-40 nucleotides in length.

In some embodiments, the GC content of the region of complementarity is about 40% to about 80%.

In some embodiments, the GC content of the region of complementarity is about 50% to about 80%.

In some embodiments, the GC content of the region of complementarity is about 60% to about 80%.

In some embodiments, the GC content of the region of complementarity is at least 63%.

In some embodiments, the GC content of the region of complementarity is 63%-79%.

In some embodiments, the GC content of the region of complementarity is no more than about 45%.

In some embodiments, the GC content of the region of complementarity is no more than 45%.

In some embodiments, the GC content of the region of complementarity is about 45%-60%.

In some embodiments, the GC content of the region of complementarity is 45%-60%.

In some embodiments, the GC content of the region of complementarity is about 60%-75%.

In some embodiments, the GC content of the region of complementarity is 60%-75%.

In some embodiments, the GC content of the region of complementarity is at least about 75%.

In some embodiments, the GC content of the region of complementarity is at least 75%.

In some embodiments, the first editing template comprises SEQ ID NO: 2691.

In some embodiments, the second editing template comprises SEQ ID NO: 3075.

In some embodiments, in the first editing template comprises a sequence selected from the group consisting of SEQ ID NOs: 2692-2736.

In some embodiments, the second editing template comprises a sequence selected from the group consisting of SEQ ID NOs: 3076-3120.

In some embodiments, the first editing template comprises a sequence selected from the group consisting of SEQ ID NOs: 2692-2700.

In some embodiments, the second editing template comprises a sequence selected from the group consisting of SEQ ID NOs: 3076-3084.

In some embodiments, the first editing template comprises a sequence selected from the group consisting of SEQ ID NOs: 2701-2709.

In some embodiments, the second editing template comprises a sequence selected from the group consisting of SEQ ID NOs: 3085-3093.

In some embodiments, the first editing template comprises a sequence selected from the group consisting of SEQ ID NOs: 2710-2718.

In some embodiments, the second editing template comprises a sequence selected from the group consisting of SEQ ID NOs: 3094-3102.

In some embodiments, the first editing template comprises a sequence selected from the group consisting of SEQ ID NOs: 2719-2727.

In some embodiments, the second editing template comprises a sequence selected from the group consisting of SEQ ID NOs: 3103-3111.

In some embodiments, the first editing template comprises a sequence selected from the group consisting of SEQ ID NOs: 2728-2736.

In some embodiments, the second editing template comprises a sequence selected from the group consisting of SEQ ID NOs: 3112-3120.

In some embodiments, the first editing template comprises a sequence selected from the group consisting of SEQ ID NOs: 2737-2769.

In some embodiments, the second editing template comprises a sequence selected from the group consisting of SEQ ID NOs: 3121-3153.

In some embodiments, the first editing template comprises a sequence selected from the group consisting of SEQ ID NOs: 2737-2745 and 2769.

In some embodiments, the second editing template comprises a sequence selected from the group consisting of SEQ ID NOs: 3121-3129 and 3153.

In some embodiments, the first editing template comprises a sequence selected from the group consisting of SEQ ID NOs: 2746-2749.

In some embodiments, the second editing template comprises a sequence selected from the group consisting of SEQ ID NOs: 3130-3133.

In some embodiments, the first editing template comprises a sequence selected from the group consisting of SEQ ID NOs: 2750-2758.

In some embodiments, the second editing template comprises a sequence selected from the group consisting of SEQ ID NOs: 3134-3142.

In some embodiments, the first editing template comprises a sequence selected from the group consisting of SEQ ID NOs: 2759-2768.

In some embodiments, the second editing template comprises a sequence selected from the group consisting of SEQ ID NOs: 3143-3152.

In some embodiments, the first editing template comprises a sequence selected from the group consisting of SEQ ID NOs: 2770-3074.

In some embodiments, the second editing template comprises a sequence selected from the group consisting of SEQ ID NOs: 3154-3458.

In some embodiments, the region of complementarity is 10 to 19 nt in length and has a GC content of at least about 75%.

In some embodiments, the first editing template comprises SEQ ID NOs: 2796 or 2804.

In some embodiments, the second editing template comprises SEQ ID NO: 3180 or 3188.

In some embodiments, the first editing template comprises a sequence selected from the group consisting of SEQ ID NOs: 2895, 3008, 3011, 2847, 2859, and 2864.

In some embodiments, the second editing template comprises a sequence selected from the group consisting of SEQ ID NOs: 3279, 3392, 3395, 3231, 3243, and 3248.

In some embodiments, the region of complementarity is 10 to 19 nt in length and has a GC content of about 45%-60%.

In some embodiments, the first editing template comprises SEQ ID NOs: 2792 or 3014.

In some embodiments, the second editing template comprises SEQ ID NOs: 3176 or 3398:

In some embodiments, first editing template comprises a sequence selected from the group consisting of SEQ ID NOs: 2819, 2786, and 2835.

In some embodiments, the second editing template comprises a sequence selected from the group consisting of SEQ ID NOs: 3203, 3170, and 3219.

In some embodiments, the region of complementarity is 10 to 19 nt in length and has a GC content of less than about 45%.

In some embodiments, the first editing template comprises SEQ ID NO: 2770 or 2803.

In some embodiments, the second editing template comprises SEQ ID NO: 3154 or 3187.

In some embodiments, the first editing template comprises a sequence selected from the group consisting of SEQ ID NOs: 2812, 2929, and 2797.

In some embodiments, the second editing template comprises a sequence selected from the group consisting of SEQ ID NOs: 3196, 3313, and 3181.

In some embodiments, the region of complementarity is 10 to 19 nt in length and has a GC content of about 60%-75%.

In some embodiments, the first editing template comprises SEQ ID NOs: 2781 or 2977.

In some embodiments, the second editing template comprises a SEQ ID NOs: 3165 or 3361.

In some embodiments, the first editing template comprises a sequence selected from the group consisting of SEQ ID NOs: 2802, 2823, 2939, 2831, and 2909.

In some embodiments, the second editing template comprises a sequence selected from the group consisting of SEQ ID NOs: 3186, 3207, 3323, 3215, and 3293.

In some embodiments, the region of complementarity is 20-30 nt in length and has a GC content of at least about 75%.

In some embodiments, n the first editing template comprises SEQ ID NO: 2851

In some embodiments, the second editing template comprises SEQ ID NO: 3235.

In some embodiments, the region of complementarity is 20-30 nt in length and has a GC content of about 45%-60%.

In some embodiments, the first editing template comprises SEQ ID NOs: 2924 or 3066.

In some embodiments, the second editing template comprises SEQ ID NOs: 3308 or 3450.

In some embodiments, first editing template comprises a sequence selected from the group consisting of SEQ ID NOs: 2925, 2995, 2950, and 2978.

In some embodiments, the second editing template comprises a sequence selected from the group consisting of SEQ ID NOs: 3309, 3379, 3334, and 3362.

In some embodiments, the region of complementarity is 20 to 30 nt in length and has a GC content of less than about 45%.

In some embodiments, first editing template comprises SEQ ID NOs: 2777 or 3032.

In some embodiments, the second editing template comprises SEQ ID NO:3161 or 3416.

In some embodiments, the first editing template comprises a sequence selected from the group consisting of SEQ ID NOs: 3040, 3072, and 2773.

In some embodiments, the second editing template comprises a sequence selected from the group consisting of SEQ ID NOs: 3424, 3456, and 3157.

In some embodiments, the region of complementarity is 20 to 30 nt in length and has a GC content of about 60%-75%.

In some embodiments, the first editing template comprises SEQ ID No 2980 or 3039.

In some embodiments, the second editing template comprises a sequence selected from the group consisting of SEQ ID NOs: 3364 or 3423.

In some embodiments, the first editing template comprises SEQ ID NOs: 2997 or 2849.

In some embodiments, the second editing template SEQ ID NOs: 3381, or 3233.

In some embodiments, the region of complementarity is at least 30 nt in length and has a GC content of about 45%-60%, optionally wherein the region of complementarity is 30 to 40 nt in length.

In some embodiments, the first editing template comprises SEQ ID NO: 3013.

In some embodiments, the second editing template comprises a sequence selected from the group consisting of SEQ ID NO: 3397.

In some embodiments, the first editing template comprises SEQ ID NO: 2820 or 2976.

In some embodiments, the second editing template comprises SEQ ID NO: 3204 or 3360.

In some embodiments, the region of complementarity is at least 30 nt in length and has a GC content of less than about 45%, optionally wherein the region of complementarity is 30 to 40 nt in length.

In some embodiments, the first editing template comprises SEQ ID NO: 2943 or 3020.

In some embodiments, the second editing template comprises a sequence selected from the group consisting of SEQ ID NOs: 3327 or 3404.

In some embodiments, the first editing template comprises SEQ ID NO: 2778 or 3061.

In some embodiments, the second editing template comprises SEQ ID NOs: 3162 or 3445.

In some embodiments, the prime editing composition of the above described embodiments comprises the first PEgRNA and the second PEgRNA, wherein: (i) the first spacer comprises SEQ ID NO: 77, and the first PBS comprises SEQ ID No: 85; (ii) the first spacer comprises SEQ ID NO: 536, and the first PBS comprises SEQ ID No: 544; or (iii) the first spacer comprises SEQ ID NO: 1019, and the first PBS comprises SEQ ID No:1027; and wherein: (i) the second spacer comprises SEQ ID NO: 1900, and the second PBS comprises SEQ ID NO: 1908; (ii) the second spacer comprises SEQ ID NO: 1936, and the second PBS comprises SEQ ID NO: 1944; or (iii) the second spacer comprises SEQ ID NO: 2673, and the second PBS comprises SEQ ID NO: 2681.

In some embodiments, the first spacer comprises SEQ ID No: 77, and the first PBS is 8-10 nucleotides in length and comprises SEQ ID NOs: 87, 86, or 85.

In some embodiments, the second spacer comprises SEQ ID NO: 1936, and the second PBS is 8-10 nucleotides in length and comprises SEQ ID NOs: 1946, 1945, or 1944.

In some embodiments, the first PEgRNA comprises a sequence selected from the group consisting of SEQ ID NOs: 93-102, 552, 553, 1035, and 1036, and wherein the second PEgRNA comprises a sequence selected from the group consisting of SEQ ID NOs: 1952-1961, 1916, 1917, 2689, and 2690.

In some embodiments, the first PEgRNA comprises a sequence selected from the group consisting of SEQ ID NOs: 94, 552, and 1035; and wherein the second PEgRNA comprises a sequence selected from the group consisting of 1952, 1916, and 2689.

In some embodiments, the first editing template and the second editing template have the same length and are perfectly complementary to each other.

In some embodiments, the first spacer comprises at its 3′ end nucleotides 5-20 of SEQ ID NO: 77, and wherein the first editing template comprises a sequence selected from the group consisting of SEQ ID NOs: 2771, 2800, 2811, 2775, 2778, 2820, 2990, 2776, 2779, 2780, 2786, 2799, 2802, 2812, 2819, 2937, 2996, 3010, 3041, 3044, 3058, 2813, 3068, 3070, and 3072.

In some embodiments, the first spacer comprises at its 3′ end nucleotides 5-20 of SEQ ID NO: 1019, and wherein the first editing template comprises a sequence selected from the group consisting of SEQ ID NOs: 2795, 2949, 2816, 2775, 2778, 2820, 2990, 2776, 2779, 2780, 2786, 2799, 2802, 2812, 2819, 2937, 2996, 3010, 3041, 2813, 3068, 3070, and 3072.

In some embodiments, the first spacer comprises at its 3′ end nucleotides 5-20 of SEQ ID NO: 590, and wherein the first editing template comprises a sequence selected from the group consisting of SEQ ID NOs: 2774, 2834, 2896, 2940, 2962, 3009, 30302816, 2776, 2779, 2780, 2786, 2799, 2802, 2812, 2819, 2937, 2996, 3010, 3041, 3044, 3058, and 3072.

In some embodiments, the second spacer comprises at its 3′ end nucleotides 5-20 of SEQ ID NO: 1525, and wherein the second editing template comprises a sequence selected from the group consisting of SEQ ID NOs: 3195, 3158, 3414, 3393, 3162, 3204, 3164, 3170, 3203, 3380, 3425, 3442, 3197, 3454, 3159, 3186, 3196, 3456, 3280, 3160, and 3163.

In some embodiments, the second spacer comprises at its 3′ end nucleotides 5-20 of SEQ ID NO: 1936, and wherein the second editing template comprises a sequence selected from the group consisting of SEQ ID NOs: 3184, 3218, 3346, 3321, 3428, 3452, 3393, 3162, 3204, 3164, 3170, 3203, 3380, 3425, 3442, 3197, 3454, 3159, 3186, 3196, and 3456.

In some embodiments, the second spacer comprises at its 3′ end nucleotides 5-20 of SEQ ID NO: 2353, and wherein the second editing template comprises a sequence selected from the group consisting of SEQ ID NOs: 3155, 3324, 3179, 3333, 3200, 3374, 3183, 3394, 3452, 3393, 3162, 3204, 3164, 3170, 3203, 3380, 3425, 3442, 3197, 3454, 3280, 3160, and 3163.

In some embodiments, the first editing template and the second editing template have the same length and are perfectly complementary to each other.

In some embodiments, the prime editing composition comprises the first PEgRNA and the second PEgRNa, wherein the first editing template comprises at its 3′ end nucleotides 8-17 of the selected sequence for the second spacer, and wherein the second editing template comprises at its 3′ end nucleotides 8-17 of the selected sequence of the first spacer.

In some embodiments, the first editing template comprises at its 3′ end nucleotides 1-17, 2-17, 3-17, 4-17, 5-17, 6-17, 7-17, or 8-17 of the selected sequence for the second spacer, and/or wherein the second editing template comprises at its 3′ end nucleotides 1-17, 2-17, 3-17, 4-17, 5-17, 6-17, 7-17, or 8-17 of the selected sequence for the first spacer.

In some embodiments, the first editing template comprises at its 3′ end nucleotides 3-17 of the selected sequence for the second spacer, and wherein the second editing template comprises at its 3′ end nucleotides 3-17 of the selected sequence of the first spacer.

In some embodiments, the first editing template comprises a region of complementarity to a sequence on the second strand of the DMPK gene that is directly 3′ to nucleotide 3 of the second search target sequence, wherein the region of complementarity is about 20, about 25, about 30 about 35, about 40, about 45, about 50 nucleotides in length.

In some embodiments, a prime editing composition comprises a first PEgRNA comprising a first editing template that comprises a region of complementarity to a sequence on the second strand of the DMPK gene that is directly 3′ to nucleotide 3 of the second search target sequence, wherein the region of complementarity is about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, or about 50 nucleotides in length.

In some embodiments, a prime editing composition of comprises a first PEgRNA comprising a first editing template that comprises a region of complementarity to a sequence on the second strand of the DMPK gene that is directly 3′ to nucleotide 3 of the second search target sequence, wherein the region of complementarity is between 20 and 50 nucleotides; between 21 and 50 nucleotides; between 22 and 50 nucleotides; between 23 and 50 nucleotides; between 24 and 50 nucleotides; between 25 and 50 nucleotides; between 26 and 50 nucleotides; between 27 and 50 nucleotides; between 28 and 50 nucleotides; between 29 and 50 nucleotides; between 30 and 50 nucleotides; between 31 and 50 nucleotides; between 32 and 50 nucleotides; between 33 and 50 nucleotides; between 34 and 50 nucleotides; between 35 and 50 nucleotides; between 36 and 50 nucleotides; between 37 and 50 nucleotides; between 38 and 50 nucleotides; between 39 and 50 nucleotides; between 40 and 50 nucleotides; between 41 and 50 nucleotides; between 42 and 50 nucleotides; between 43 and 50 nucleotides; between 44 and 50 nucleotides; between 45 and 50 nucleotides; between 46 and 50 nucleotides; between 47 and 50 nucleotides; between 48 and 50 nucleotides; between 20 and 50 nucleotides; between 20 and 49 nucleotides; between 20 and 48 nucleotides; between 20 and 47 nucleotides; between 20 and 46 nucleotides; between 20 and 45 nucleotides; between 20 and 44 nucleotides; between 20 and 43 nucleotides; between 20 and 42 nucleotides; between 20 and 41 nucleotides; between 20 and 40 nucleotides; between 20 and 39 nucleotides; between 20 and 38 nucleotides; between 20 and 37 nucleotides; between 20 and 36 nucleotides; between 20 and 35 nucleotides; between 20 and 34 nucleotides; between 20 and 33 nucleotides; between 20 and 32 nucleotides; between 20 and 31 nucleotides; between 20 and 30 nucleotides; between 20 and 29 nucleotides; between 20 and 28 nucleotides; between 20 and 27 nucleotides; between 20 and 26 nucleotides; between 20 and 25 nucleotides; between 20 and 24 nucleotides; between 20 and 23 nucleotides; or between 20 and 22 nucleotides in length.

In some embodiments, a prime editing composition of a first PEgRNA that comprises a first editing template that comprises a region of complementarity to a sequence on the second strand of the DMPK gene that is directly 3′ to nucleotide 3 of the second search target sequence, wherein the region of complementarity is at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, or at least 50 nucleotides in length.

In some embodiments, a prime editing composition of comprises a first PEgRNA that comprises a first editing template that comprises a region of complementarity to a sequence on the second strand of the DMPK gene that is directly 3′ to nucleotide 3 of the second search target sequence, wherein the region of complementarity is no more than 20, no more than 21, no more than 22, no more than 23, no more than 24, no more than 25, no more than 26, no more than 27, no more than 28, no more than 29, no more than 30, no more than 31, no more than 32, no more than 33, no more than 34, no more than 35, no more than 36, no more than 37, no more than 38, no more than 39, no more than 40, no more than 41, no more than 42, no more than 43, no more than 44, no more than 45, no more than 46, no more than 47, no more than 48, no more than 49, or no more than 50 nucleotides in length.

In some embodiments, the region of complementarity is about 20-30 nucleotides in length.

In some embodiments, the second editing template comprises a region of complementarity to a sequence on the first strand of the DMPK gene that is directly 3′ to nucleotide 3 of the first search target sequence, wherein the region of complementarity is about 20, about 25, about 30, about 35, about 40, about 45, or about 50 nucleotides in length.

In some embodiments, the region of complementarity is about 20-30 nucleotides in length.

In some embodiments, the first editing template comprises a sequence selected from the group consisting of SEQ ID NOs: 3459-3461, wherein the second spacer comprises at its 3′ end nucleotides 5-20 of SEQ ID NO: 1525, optionally wherein the second spacer comprises at its 3 end SEQ ID NO: 1525.

In some embodiments, the first editing template comprises a sequence selected from the group consisting of SEQ ID NOs: 3467-3471, wherein the second spacer comprises at its 3′ end nucleotides 5-20 of SEQ ID NO: 1525, optionally wherein the second spacer comprises at its 3 end SEQ ID NO: 1525.

In some embodiments, the first editing template comprises a sequence selected from the group consisting of SEQ ID NOs: 3462-3466, and wherein the second spacer comprises at its 3′ end nucleotides 5-20 of SEQ ID NO: 2353, optionally wherein the second spacer comprises at its 3 end SEQ ID NO: 2353.

In some embodiments, the first editing template comprises a sequence selected from the group consisting of SEQ ID NOs: 3472-3476, wherein the second spacer comprises at its 3′ end nucleotides 5-20 of SEQ ID NO: 1936, optionally wherein the second spacer comprises at its 3 end SEQ ID NO: 1936.

In some embodiments, the first editing template comprises nucleotides 31-40, 30-40, 29-40, 28-40, 27-40, 26-40, 25-40, 24-40, 23-40, 22-40, 21-40, 20-40, 19-40, 18-40, 17-40, 16-40, 15-40, 14-40, 13-40, 12-40, 11-40, 10-40, 9-40, 8-40, 7-40, 6-40, 5-40, 4-40, 3-40, 2-40, or 1-40 of SEQ ID NO: 3647, wherein the second spacer comprises at its 3′ end nucleotides 5-20 of SEQ ID NO:1900, optionally wherein the second spacer comprises at its 3′ end SEQ ID NO: 1900.

In some embodiments, the first editing template comprises nucleotides 31-40, 30-40, 29-40, 28-40, 27-40, 26-40, 25-40, 24-40, 23-40, 22-40, 21-40, 20-40, 19-40, 18-40, 17-40, 16-40, 15-40, 14-40, 13-40, 12-40, 11-40, 10-40, 9-40, 8-40, 7-40, 6-40, 5-40, 4-40, 3-40, 2-40, or 1-40 of SEQ ID NO: 3648, wherein the second spacer comprises at its 3′ end nucleotides 5-20 of SEQ ID NO: 1918, optionally wherein the second spacer comprises at its 3′ end SEQ ID NO: 1918.

In some embodiments, the first editing template comprises nucleotides 31-40, 30-40, 29-40, 28-40, 27-40, 26-40, 25-40, 24-40, 23-40, 22-40, 21-40, 20-40, 19-40, 18-40, 17-40, 16-40, 15-40, 14-40, 13-40, 12-40, 11-40, 10-40, 9-40, 8-40, 7-40, 6-40, 5-40, 4-40, 3-40, 2-40, or 1-40 of SEQ ID NO: 3649, wherein the second spacer comprises at its 3′ end nucleotides 5-20 of SEQ ID NO: 1403, optionally wherein the second spacer comprises at its 3′ end SEQ ID NO: 1403.

In some embodiments, the first editing template comprises nucleotides 31-40, 30-40, 29-40, 28-40, 27-40, 26-40, 25-40, 24-40, 23-40, 22-40, 21-40, 20-40, 19-40, 18-40, 17-40, 16-40, 15-40, 14-40, 13-40, 12-40, 11-40, 10-40, 9-40, 8-40, 7-40, 6-40, 5-40, 4-40, 3-40, 2-40, or 1-40 of SEQ ID NO: 3650, wherein the second spacer comprises at its 3′ end nucleotides 5-20 of SEQ ID NO: 1936, optionally wherein the second spacer comprises at its 3′ end SEQ ID NO: 1936.

In some embodiments, the first editing template comprises nucleotides 31-40, 30-40, 29-40, 28-40, 27-40, 26-40, 25-40, 24-40, 23-40, 22-40, 21-40, 20-40, 19-40, 18-40, 17-40, 16-40, 15-40, 14-40, 13-40, 12-40, 11-40, 10-40, 9-40, 8-40, 7-40, 6-40, 5-40, 4-40, 3-40, 2-40, or 1-40 of SEQ ID NO: 3651, wherein the second spacer comprises at its 3′ end nucleotides 5-20 of SEQ ID NO: 2263, optionally wherein the second spacer comprises at its 3′ end SEQ ID NO: 2263.

In some embodiments, the first editing template comprises nucleotides 31-40, 30-40, 29-40, 28-40, 27-40, 26-40, 25-40, 24-40, 23-40, 22-40, 21-40, 20-40, 19-40, 18-40, 17-40, 16-40, 15-40, 14-40, 13-40, 12-40, 11-40, 10-40, 9-40, 8-40, 7-40, 6-40, 5-40, 4-40, 3-40, 2-40, or 1-40 of SEQ ID NO: 3652, wherein the second spacer comprises at its 3′ end nucleotides 5-20 of SEQ ID NO: 2353, optionally wherein the second spacer comprises at its 3′ end SEQ ID NO: 2353.

In some embodiments, the first editing template comprises nucleotides 31-40, 30-40, 29-40, 28-40, 27-40, 26-40, 25-40, 24-40, 23-40, 22-40, 21-40, 20-40, 19-40, 18-40, 17-40, 16-40, 15-40, 14-40, 13-40, 12-40, 11-40, 10-40, 9-40, 8-40, 7-40, 6-40, 5-40, 4-40, 3-40, 2-40, or 1-40 of SEQ ID NO: 3653, wherein the second spacer comprises at its 3′ end nucleotides 5-20 of SEQ ID NO: 1503, optionally wherein the second spacer comprises at its 3′ end SEQ ID NO: 1503.

In some embodiments, the first editing template comprises nucleotides 31-40, 30-40, 29-40, 28-40, 27-40, 26-40, 25-40, 24-40, 23-40, 22-40, 21-40, 20-40, 19-40, 18-40, 17-40, 16-40, 15-40, 14-40, 13-40, 12-40, 11-40, 10-40, 9-40, 8-40, 7-40, 6-40, 5-40, 4-40, 3-40, 2-40, or 1-40 of SEQ ID NO: 3654, wherein the second spacer comprises at its 3′ end nucleotides 5-20 of SEQ ID NO: 2281, optionally wherein the second spacer comprises at its 3′ end SEQ ID NO: 2281.

In some embodiments, the first editing template comprises nucleotides 31-40, 30-40, 29-40, 28-40, 27-40, 26-40, 25-40, 24-40, 23-40, 22-40, 21-40, 20-40, 19-40, 18-40, 17-40, 16-40, 15-40, 14-40, 13-40, 12-40, 11-40, 10-40, 9-40, 8-40, 7-40, 6-40, 5-40, 4-40, 3-40, 2-40, or 1-40 of SEQ ID NO: 3655, wherein the second spacer comprises at its 3′ end nucleotides 5-20 of SEQ ID NO: 2673, optionally wherein the second spacer comprises at its 3′ end SEQ ID NO: 2673.

In some embodiments, the first editing template comprises nucleotides 31-40, 30-40, 29-40, 28-40, 27-40, 26-40, 25-40, 24-40, 23-40, 22-40, 21-40, 20-40, 19-40, 18-40, 17-40, 16-40, 15-40, 14-40, 13-40, 12-40, 11-40, 10-40, 9-40, 8-40, 7-40, 6-40, 5-40, 4-40, 3-40, 2-40, or 1-40 of SEQ ID NO: 3656, wherein the second spacer comprises at its 3′ end nucleotides 5-20 of SEQ ID NO: 1525, optionally wherein the second spacer comprises at its 3′ end SEQ ID NO: 1525.

In some embodiments, the first editing template comprises nucleotides 31-40, 30-40, 29-40, 28-40, 27-40, 26-40, 25-40, 24-40, 23-40, 22-40, 21-40, 20-40, 19-40, 18-40, 17-40, 16-40, 15-40, 14-40, 13-40, 12-40, 11-40, 10-40, 9-40, 8-40, 7-40, 6-40, 5-40, 4-40, 3-40, 2-40, or 1-40 of SEQ ID NO: 3657, wherein the second spacer comprises at its 3′ end nucleotides 5-20 of SEQ ID NO: 2299, optionally wherein the second spacer comprises at its 3′ end SEQ ID NO: 2299.

In some embodiments, the first editing template comprises nucleotides 31-40, 30-40, 29-40, 28-40, 27-40, 26-40, 25-40, 24-40, 23-40, 22-40, 21-40, 20-40, 19-40, 18-40, 17-40, 16-40, 15-40, 14-40, 13-40, 12-40, 11-40, 10-40, 9-40, 8-40, 7-40, 6-40, 5-40, 4-40, 3-40, 2-40, or 1-40 of SEQ ID NO: 3658, wherein the second spacer comprises at its 3′ end nucleotides 5-20 of SEQ ID NO: 2317, optionally wherein the second spacer comprises at its 3′ end SEQ ID NO: 2317.

In some embodiments, the first editing template comprises nucleotides 31-40, 30-40, 29-40, 28-40, 27-40, 26-40, 25-40, 24-40, 23-40, 22-40, 21-40, 20-40, 19-40, 18-40, 17-40, 16-40, 15-40, 14-40, 13-40, 12-40, 11-40, 10-40, 9-40, 8-40, 7-40, 6-40, 5-40, 4-40, 3-40, 2-40, or 1-40 of SEQ ID NO: 3659, wherein the second spacer comprises at its 3′ end nucleotides 5-20 of SEQ ID NO: 1864, optionally wherein the second spacer comprises at its 3′ end SEQ ID NO: 1864.

In some embodiments, the first editing template comprises nucleotides 31-40, 30-40, 29-40, 28-40, 27-40, 26-40, 25-40, 24-40, 23-40, 22-40, 21-40, 20-40, 19-40, 18-40, 17-40, 16-40, 15-40, 14-40, 13-40, 12-40, 11-40, 10-40, 9-40, 8-40, 7-40, 6-40, 5-40, 4-40, 3-40, 2-40, or 1-40 of SEQ ID NO: 3660, wherein the second spacer comprises at its 3′ end nucleotides 5-20 of SEQ ID NO: 1359, optionally wherein the second spacer comprises at its 3′ end SEQ ID NO: 1359.

In some embodiments, the first editing template comprises nucleotides 31-40, 30-40, 29-40, 28-40, 27-40, 26-40, 25-40, 24-40, 23-40, 22-40, 21-40, 20-40, 19-40, 18-40, 17-40, 16-40, 15-40, 14-40, 13-40, 12-40, 11-40, 10-40, 9-40, 8-40, 7-40, 6-40, 5-40, 4-40, 3-40, 2-40, or 1-40 of SEQ ID NO: 3661, wherein the second spacer comprises at its 3′ end nucleotides 5-20 of SEQ ID NO: 2335, optionally wherein the second spacer comprises at its 3′ end SEQ ID NO: 2335.

In some embodiments, the first editing template comprises nucleotides 31-40, 30-40, 29-40, 28-40, 27-40, 26-40, 25-40, 24-40, 23-40, 22-40, 21-40, 20-40, 19-40, 18-40, 17-40, 16-40, 15-40, 14-40, 13-40, 12-40, 11-40, 10-40, 9-40, 8-40, 7-40, 6-40, 5-40, 4-40, 3-40, 2-40, or 1-40 of SEQ ID NO: 3662, wherein the second spacer comprises at its 3′ end nucleotides 5-20 of SEQ ID NO: 1882, optionally wherein the second spacer comprises at its 3′ end SEQ ID NO: 1882.

In some embodiments, the first editing template comprises nucleotides 31-40, 30-40, 29-40, 28-40, 27-40, 26-40, 25-40, 24-40, 23-40, 22-40, 21-40, 20-40, 19-40, 18-40, 17-40, 16-40, 15-40, 14-40, 13-40, 12-40, 11-40, 10-40, 9-40, 8-40, 7-40, 6-40, 5-40, 4-40, 3-40, 2-40, or 1-40 of SEQ ID NO: 3663, wherein the second spacer comprises at its 3′ end nucleotides 5-20 of SEQ ID NO: 1381, optionally wherein the second spacer comprises at its 3′ end SEQ ID NO: 1381.

In some embodiments, the second editing template comprises a sequence selected from the group consisting of SEQ ID NOs: 3477-3479, wherein the first spacer comprises at its 3′ end nucleotides 5-20 of SEQ ID NO: 484, optionally wherein the first spacer comprises at its 3′ end SEQ ID NO: 484.

In some embodiments, the second editing template comprises a sequence selected from the group consisting of SEQ ID NOs: 3480-3484, wherein the first spacer comprises at its 3′ end nucleotides 5-20 of SEQ ID NO: 77, optionally wherein the first spacer comprises at its 3 end SEQ ID NO: 77.

In some embodiments, the second editing template comprises a sequence selected from the group consisting of SEQ ID NOs: 3485-3489, wherein the first spacer comprises at its 3′ end nucleotides 5-20 of SEQ ID NO: 590, optionally wherein the first spacer comprises at its 3 end SEQ ID NO: 590.

In some embodiments, the second editing template comprises a sequence selected from the group consisting of SEQ ID NOs: 3490-3494, wherein the first spacer comprises at its 3′ end nucleotides 5-20 of SEQ ID NO: 1019, optionally wherein the first spacer comprises at its 3 end SEQ ID NO: 1019.

In some embodiments, the second editing template comprises nucleotides 31-40, 30-40, 29-40, 28-40, 27-40, 26-40, 25-40, 24-40, 23-40, 22-40, 21-40, 20-40, 19-40, 18-40, 17-40, 16-40, 15-40, 14-40, 13-40, 12-40, 11-40, 10-40, 9-40, 8-40, 7-40, 6-40, 5-40, 4-40, 3-40, 2-40, or 1-40 of SEQ ID NO: 3664, wherein the second spacer comprises at its 3′ end nucleotides 5-20 of SEQ ID NO:518, optionally wherein the second spacer comprises at its 3′ end SEQ ID NO:518.

In some embodiments, the second editing template comprises nucleotides 31-40, 30-40, 29-40, 28-40, 27-40, 26-40, 25-40, 24-40, 23-40, 22-40, 21-40, 20-40, 19-40, 18-40, 17-40, 16-40, 15-40, 14-40, 13-40, 12-40, 11-40, 10-40, 9-40, 8-40, 7-40, 6-40, 5-40, 4-40, 3-40, 2-40, or 1-40 of SEQ ID NO:3665, wherein the second spacer comprises at its 3′ end nucleotides 5-20 of SEQ ID NO:536, optionally wherein the second spacer comprises at its 3′ end SEQ ID NO:536.

Embodiment 58f: The prime editing composition of any one of Embodiments 50-58, wherein the second editing template comprises nucleotides 31-40, 30-40, 29-40, 28-40, 27-40, 26-40, 25-40, 24-40, 23-40, 22-40, 21-40, 20-40, 19-40, 18-40, 17-40, 16-40, 15-40, 14-40, 13-40, 12-40, 11-40, 10-40, 9-40, 8-40, 7-40, 6-40, 5-40, 4-40, 3-40, 2-40, or 1-40 of SEQ ID NO:3666, wherein the second spacer comprises at its 3′ end nucleotides 5-20 of SEQ ID NO:554, optionally wherein the second spacer comprises at its 3′ end SEQ ID NO:554.

In some embodiments, the second editing template comprises nucleotides 31-40, 30-40, 29-40, 28-40, 27-40, 26-40, 25-40, 24-40, 23-40, 22-40, 21-40, 20-40, 19-40, 18-40, 17-40, 16-40, 15-40, 14-40, 13-40, 12-40, 11-40, 10-40, 9-40, 8-40, 7-40, 6-40, 5-40, 4-40, 3-40, 2-40, or 1-40 of SEQ ID NO:3667, wherein the second spacer comprises at its 3′ end nucleotides 5-20 of SEQ ID NO:572, optionally wherein the second spacer comprises at its 3′ end SEQ ID NO:572.

In some embodiments, the second editing template comprises nucleotides 31-40, 30-40, 29-40, 28-40, 27-40, 26-40, 25-40, 24-40, 23-40, 22-40, 21-40, 20-40, 19-40, 18-40, 17-40, 16-40, 15-40, 14-40, 13-40, 12-40, 11-40, 10-40, 9-40, 8-40, 7-40, 6-40, 5-40, 4-40, 3-40, 2-40, or 1-40 of SEQ ID NO:3668, wherein the second spacer comprises at its 3′ end nucleotides 5-20 of SEQ ID NO:590, optionally wherein the second spacer comprises at its 3′ end SEQ ID NO:590.

In some embodiments, the second editing template comprises nucleotides 31-40, 30-40, 29-40, 28-40, 27-40, 26-40, 25-40, 24-40, 23-40, 22-40, 21-40, 20-40, 19-40, 18-40, 17-40, 16-40, 15-40, 14-40, 13-40, 12-40, 11-40, 10-40, 9-40, 8-40, 7-40, 6-40, 5-40, 4-40, 3-40, 2-40, or 1-40 of SEQ ID NO:3669, wherein the second spacer comprises at its 3′ end nucleotides 5-20 of SEQ ID NO:39, optionally wherein the second spacer comprises at its 3′ end SEQ ID NO:39.

In some embodiments, the second editing template comprises nucleotides 31-40, 30-40, 29-40, 28-40, 27-40, 26-40, 25-40, 24-40, 23-40, 22-40, 21-40, 20-40, 19-40, 18-40, 17-40, 16-40, 15-40, 14-40, 13-40, 12-40, 11-40, 10-40, 9-40, 8-40, 7-40, 6-40, 5-40, 4-40, 3-40, 2-40, or 1-40 of SEQ ID NO:3670, wherein the second spacer comprises at its 3′ end nucleotides 5-20 of SEQ ID NO:912, optionally wherein the second spacer comprises at its 3′ end SEQ ID NO:912.

In some embodiments, the second editing template comprises nucleotides 31-40, 30-40, 29-40, 28-40, 27-40, 26-40, 25-40, 24-40, 23-40, 22-40, 21-40, 20-40, 19-40, 18-40, 17-40, 16-40, 15-40, 14-40, 13-40, 12-40, 11-40, 10-40, 9-40, 8-40, 7-40, 6-40, 5-40, 4-40, 3-40, 2-40, or 1-40 of SEQ ID NO:3671, wherein the second spacer comprises at its 3′ end nucleotides 5-20 of SEQ ID NO:929, optionally wherein the second spacer comprises at its 3′ end SEQ ID NO:929.

In some embodiments, the second editing template comprises nucleotides 31-40, 30-40, 29-40, 28-40, 27-40, 26-40, 25-40, 24-40, 23-40, 22-40, 21-40, 20-40, 19-40, 18-40, 17-40, 16-40, 15-40, 14-40, 13-40, 12-40, 11-40, 10-40, 9-40, 8-40, 7-40, 6-40, 5-40, 4-40, 3-40, 2-40, or 1-40 of SEQ ID NO:3672, wherein the second spacer comprises at its 3′ end nucleotides 5-20 of SEQ ID NO:947, optionally wherein the second spacer comprises at its 3′ end SEQ ID NO:947.

In some embodiments, the second editing template comprises nucleotides 31-40, 30-40, 29-40, 28-40, 27-40, 26-40, 25-40, 24-40, 23-40, 22-40, 21-40, 20-40, 19-40, 18-40, 17-40, 16-40, 15-40, 14-40, 13-40, 12-40, 11-40, 10-40, 9-40, 8-40, 7-40, 6-40, 5-40, 4-40, 3-40, 2-40, or 1-40 of SEQ ID NO:3673, wherein the second spacer comprises at its 3′ end nucleotides 5-20 of SEQ ID NO:965, optionally wherein the second spacer comprises at its 3′ end SEQ ID NO:965.

In some embodiments, the second editing template comprises nucleotides 31-40, 30-40, 29-40, 28-40, 27-40, 26-40, 25-40, 24-40, 23-40, 22-40, 21-40, 20-40, 19-40, 18-40, 17-40, 16-40, 15-40, 14-40, 13-40, 12-40, 11-40, 10-40, 9-40, 8-40, 7-40, 6-40, 5-40, 4-40, 3-40, 2-40, or 1-40 of SEQ ID NO:3674, wherein the second spacer comprises at its 3′ end nucleotides 5-20 of SEQ ID NO:983, optionally wherein the second spacer comprises at its 3′ end SEQ ID NO:983.

In some embodiments, the second editing template comprises nucleotides 31-40, 30-40, 29-40, 28-40, 27-40, 26-40, 25-40, 24-40, 23-40, 22-40, 21-40, 20-40, 19-40, 18-40, 17-40, 16-40, 15-40, 14-40, 13-40, 12-40, 11-40, 10-40, 9-40, 8-40, 7-40, 6-40, 5-40, 4-40, 3-40, 2-40, or 1-40 of SEQ ID NO: 3675, wherein the second spacer comprises at its 3′ end nucleotides 5-20 of SEQ ID NO:1001, optionally wherein the second spacer comprises at its 3′ end SEQ ID NO:1001.

In some embodiments, the second editing template comprises nucleotides 31-40, 30-40, 29-40, 28-40, 27-40, 26-40, 25-40, 24-40, 23-40, 22-40, 21-40, 20-40, 19-40, 18-40, 17-40, 16-40, 15-40, 14-40, 13-40, 12-40, 11-40, 10-40, 9-40, 8-40, 7-40, 6-40, 5-40, 4-40, 3-40, 2-40, or 1-40 of SEQ ID NO:3676, wherein the second spacer comprises at its 3′ end nucleotides 5-20 of SEQ ID NO:1019, optionally wherein the second spacer comprises at its 3′ end SEQ ID NO:1019.

In some embodiments, the second editing template comprises nucleotides 31-40, 30-40, 29-40, 28-40, 27-40, 26-40, 25-40, 24-40, 23-40, 22-40, 21-40, 20-40, 19-40, 18-40, 17-40, 16-40, 15-40, 14-40, 13-40, 12-40, 11-40, 10-40, 9-40, 8-40, 7-40, 6-40, 5-40, 4-40, 3-40, 2-40, or 1-40 of SEQ ID NO:3677, wherein the second spacer comprises at its 3′ end nucleotides 5-20 of SEQ ID NO:1341, optionally wherein the second spacer comprises at its 3′ end SEQ ID NO:1341.

In some embodiments, the second editing template comprises nucleotides 31-40, 30-40, 29-40, 28-40, 27-40, 26-40, 25-40, 24-40, 23-40, 22-40, 21-40, 20-40, 19-40, 18-40, 17-40, 16-40, 15-40, 14-40, 13-40, 12-40, 11-40, 10-40, 9-40, 8-40, 7-40, 6-40, 5-40, 4-40, 3-40, 2-40, or 1-40 of SEQ ID NO:3678, wherein the second spacer comprises at its 3′ end nucleotides 5-20 of SEQ ID NO:20, optionally wherein the second spacer comprises at its 3′ end SEQ ID NO:20.

In some embodiments, the second editing template comprises nucleotides 31-40, 30-40, 29-40, 28-40, 27-40, 26-40, 25-40, 24-40, 23-40, 22-40, 21-40, 20-40, 19-40, 18-40, 17-40, 16-40, 15-40, 14-40, 13-40, 12-40, 11-40, 10-40, 9-40, 8-40, 7-40, 6-40, 5-40, 4-40, 3-40, 2-40, or 1-40 of SEQ ID NO:3679, wherein the second spacer comprises at its 3′ end nucleotides 5-20 of SEQ ID NO:77, optionally wherein the second spacer comprises at its 3′ end SEQ ID NO:77.

In some embodiments, the second editing template comprises nucleotides 31-40, 30-40, 29-40, 28-40, 27-40, 26-40, 25-40, 24-40, 23-40, 22-40, 21-40, 20-40, 19-40, 18-40, 17-40, 16-40, 15-40, 14-40, 13-40, 12-40, 11-40, 10-40, 9-40, 8-40, 7-40, 6-40, 5-40, 4-40, 3-40, 2-40, or 1-40 of SEQ ID NO:3680, wherein the second spacer comprises at its 3′ end nucleotides 5-20 of SEQ ID NO:484, optionally wherein the second spacer comprises at its 3′ end SEQ ID NO:484.

In some embodiments, the second editing template comprises nucleotides 31-40, 30-40, 29-40, 28-40, 27-40, 26-40, 25-40, 24-40, 23-40, 22-40, 21-40, 20-40, 19-40, 18-40, 17-40, 16-40, 15-40, 14-40, 13-40, 12-40, 11-40, 10-40, 9-40, 8-40, 7-40, 6-40, 5-40, 4-40, 3-40, 2-40, or 1-40 of SEQ ID NO:3681, wherein the second spacer comprises at its 3′ end nucleotides 5-20 of SEQ ID NO:58, optionally wherein the second spacer comprises at its 3′ end SEQ ID NO:58.

In some embodiments, the second editing template comprises nucleotides 31-40, 30-40, 29-40, 28-40, 27-40, 26-40, 25-40, 24-40, 23-40, 22-40, 21-40, 20-40, 19-40, 18-40, 17-40, 16-40, 15-40, 14-40, 13-40, 12-40, 11-40, 10-40, 9-40, 8-40, 7-40, 6-40, 5-40, 4-40, 3-40, 2-40, or 1-40 of SEQ ID NO:3682, wherein the second spacer comprises at its 3′ end nucleotides 5-20 of SEQ ID NO:1, optionally wherein the second spacer comprises at its 3′ end SEQ ID NO:1.

In some embodiments, the first PEgRNA comprises a sequence selected from the group consisting of SEQ ID NOs: 503-517, and wherein the second PEgRNA comprises a sequence selected from the group consisting of SEQ ID NOs: 1548-1562.

In some embodiments, the prime editing composition comprises the first PEgRNA and the second PEgRNA, wherein the first editing template comprises from 5′ to 3′ (i) a region of complementarity to the second editing template and (ii) nucleotides 8-17 of the selected sequence for the second spacer; and wherein the second editing template comprises from 5′ to 3′ (i) a region of complementarity to the first editing template and (ii) nucleotides 8-17 of the selected sequence for the first spacer.

In some embodiments, the first editing template comprises nucleotides 1-17, 2-17, 3-17, 4-17, 5-17, 6-17, 7-17, or 8-17 of the selected sequence for the second spacer, and/or wherein the second editing template comprises 1-17, 2-17, 3-17, 4-17, 5-17, 6-17, 7-17, or 8-17 of the selected sequence for the first spacer.

In some embodiments, the first editing template comprises nucleotides 3-17 of the selected sequence for the second spacer, and wherein the second editing template comprises nucleotides 3-17 of the selected sequence of the first spacer.

In some embodiments, the first editing template comprises a region of complementarity to a sequence on the second strand of the DMPK gene that is directly 3′ to nucleotide 3 of the second search target sequence, wherein the region of complementarity is about 20, about 25, about 30, about 35, about 40, about 45, or about 50 nucleotides in length.

In some embodiments, the region of complementarity between the first editing template and the sequence on the second strand of the DMPK gene is about 20-30 nucleotides in length.

In some embodiments, the second editing template comprises a region of complementarity to a sequence on the first strand of the DMPK gene that is directly 3′ to nucleotide 3 of the first search target sequence, wherein the region of complementarity is about 20, about 25, about 30, about 35, about 40, about 45, or about 50 nucleotides in length.

In some embodiments, the region of complementarity between the second editing template and the sequence on the first strand of the DMPK gene is about 20-30 nucleotides in length.

In some embodiments, the region of complementarity between the first editing template and the second editing template is about 20 to about 80 nucleotides in length.

In some embodiments, the prime editing composition comprises a region of complementarity between the first editing template and the second editing template that is about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, or about 80 nucleotides in length.

In some embodiments, the prime editing composition of comprises a region of complementarity between the first editing template and the second editing template that is between 20 and 80 nucleotides; between 21 and 80 nucleotides; between 22 and 80 nucleotides; between 23 and 80 nucleotides; between 24 and 80 nucleotides; between 25 and 80 nucleotides; between 26 and 80 nucleotides; between 27 and 80 nucleotides; between 28 and 80 nucleotides; between 29 and 80 nucleotides; between 30 and 80 nucleotides; between 31 and 80 nucleotides; between 32 and 80 nucleotides; between 33 and 80 nucleotides; between 34 and 80 nucleotides; between 35 and 80 nucleotides; between 36 and 80 nucleotides; between 37 and 80 nucleotides; between 38 and 80 nucleotides; between 39 and 80 nucleotides; between 40 and 80 nucleotides; between 41 and 80 nucleotides; between 42 and 80 nucleotides; between 43 and 80 nucleotides; between 44 and 80 nucleotides; between 45 and 80 nucleotides; between 46 and 80 nucleotides; between 47 and 80 nucleotides; between 48 and 80 nucleotides; between 49 and 80 nucleotides; between 50 and 80 nucleotides; between 51 and 80 nucleotides; between 52 and 80 nucleotides; between 53 and 80 nucleotides; between 54 and 80 nucleotides; between 55 and 80 nucleotides; between 56 and 80 nucleotides; between 57 and 80 nucleotides; between 58 and 80 nucleotides; between 59 and 80 nucleotides; between 60 and 80 nucleotides; between 61 and 80 nucleotides; between 62 and 80 nucleotides; between 63 and 80 nucleotides; between 64 and 80 nucleotides; between 65 and 80 nucleotides; between 66 and 80 nucleotides; between 67 and 80 nucleotides; between 68 and 80 nucleotides; between 69 and 80 nucleotides; between 70 and 80 nucleotides; between 71 and 80 nucleotides; between 72 and 80 nucleotides; between 73 and 80 nucleotides; between 74 and 80 nucleotides; between 75 and 80 nucleotides; between 76 and 80 nucleotides; between 77 and 80 nucleotides; between 78 and 80 nucleotides; between 20 and 80 nucleotides; between 20 and 79 nucleotides; between 20 and 78 nucleotides; between 20 and 77 nucleotides; between 20 and 76 nucleotides; between 20 and 75 nucleotides; between 20 and 74 nucleotides; between 20 and 73 nucleotides; between 20 and 72 nucleotides; between 20 and 71 nucleotides; between 20 and 70 nucleotides; between 20 and 69 nucleotides; between 20 and 68 nucleotides; between 20 and 67 nucleotides; between 20 and 66 nucleotides; between 20 and 65 nucleotides; between 20 and 64 nucleotides; between 20 and 63 nucleotides; between 20 and 62 nucleotides; between 20 and 61 nucleotides; between 20 and 60 nucleotides; between 20 and 59 nucleotides; between 20 and 58 nucleotides; between 20 and 57 nucleotides; between 20 and 56 nucleotides; between 20 and 55 nucleotides; between 20 and 54 nucleotides; between 20 and 53 nucleotides; between 20 and 52 nucleotides; between 20 and 51 nucleotides; between 20 and 50 nucleotides; between 20 and 49 nucleotides; between 20 and 48 nucleotides; between 20 and 47 nucleotides; between 20 and 46 nucleotides; between 20 and 45 nucleotides; between 20 and 44 nucleotides; between 20 and 43 nucleotides; between 20 and 42 nucleotides; between 20 and 41 nucleotides; between 20 and 40 nucleotides; between 20 and 39 nucleotides; between 20 and 38 nucleotides; between 20 and 37 nucleotides; between 20 and 36 nucleotides; between 20 and 35 nucleotides; between 20 and 34 nucleotides; between 20 and 33 nucleotides; between 20 and 32 nucleotides; between 20 and 31 nucleotides; between 20 and 30 nucleotides; between 20 and 29 nucleotides; between 20 and 28 nucleotides; between 20 and 27 nucleotides; between 20 and 26 nucleotides; between 20 and 25 nucleotides; between 20 and 24 nucleotides; between 20 and 23 nucleotides; or between 20 and 22 nucleotides in length.

In some embodiments, the prime editing composition comprises a region of complementarity between the first editing template and the second editing template that is at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, or at least 80 nucleotides in length.

In some embodiments, the prime editing composition comprises a region of complementarity between the first editing template and the second editing template that is no more than 20, no more than 21, no more than 22, no more than 23, no more than 24, no more than 25, no more than 26, no more than 27, no more than 28, no more than 29, no more than 30, no more than 31, no more than 32, no more than 33, no more than 34, no more than 35, no more than 36, no more than 37, no more than 38, no more than 39, no more than 40, no more than 41, no more than 42, no more than 43, no more than 44, no more than 45, no more than 46, no more than 47, no more than 48, no more than 49, no more than 50, no more than 51, no more than 52, no more than 53, no more than 54, no more than 55, no more than 56, no more than 57, no more than 58, no more than 59, no more than 60, no more than 61, no more than 62, no more than 63, no more than 64, no more than 65, no more than 66, no more than 67, no more than 68, no more than 69, no more than 70, no more than 71, no more than 72, no more than 73, no more than 74, no more than 75, no more than 76, no more than 77, no more than 78, no more than 79, or no more than 80 nucleotides in length.

In some embodiments, the region of complementarity between the first editing template and the second editing template is about 20 to about 40 nucleotides in length.

In some embodiments, the region of complementarity between the first editing template and the second editing template is 23-38 nucleotides in length.

In an aspect, a prime editing composition comprises a first prime editing guide RNA (PEgRNA) or one or more polynucleotides encoding the first PEgRNA and a second PEgRNA or one or more polynucleotides encoding the second PEgRNA, wherein (a) the first PEgRNA comprises: (i) a first spacer comprising at its 3′ end nucleotides 5-20 of a Spacer sequence selected from any one of Tables 1A, 2A, 3A, 4A, 5A, 6A, 7A, 8A, 9A, 10A, 11A, 12A, 13A, 14A, 15A, 16A, 17A, 18A, and 19A; (ii) a first gRNA core capable of binding to a Cas9 protein; (iii) a first extension arm comprising: a first primer binding site (PBS) comprising a PBS sequence selected from the same Table as the first spacer, and a first editing template comprising an RTT sequence selected from Table 37 and has an RTT Paring NO: x in Table 37; and (b) the second PEgRNA comprises: (i) a second spacer comprising at its 3′ end nucleotides 5-20 of a Spacer sequence selected from any one of Tables 20A, 21A, 22A, 23A, 24A, 25A, 26A, 27A, 28A, 29A, 30A, 31A, 32A, 33A, 34A, 35A, and 36A; (ii) a second gRNA core capable of binding to a Cas9 protein; (iii) a second extension arm comprising: a second primer binding site (PBS) comprising a PBS sequence selected from the same Table as the second spacer, and a second editing template comprising an RTT sequence selected from Table 38 and has an RTT paring NO: x in Table 38; wherein the RTT paring number x in (a) and (b) are the same integer.

In some embodiments, the first editing template and the second editing template have the same length and are perfectly complementary to each other.

In an aspect, a prime editing composition comprising a first prime editing guide RNA (PEgRNA) or a one or more polynucleotides encoding the first PEgRNA and a second PEgRNA or one or more polynucleotides encoding the second PEgRNA, wherein (a) the first PEgRNA comprises: (i) a first spacer comprising at its 3′ end nucleotides 5-20 of a Spacer sequence selected from Table xA, wherein x is an integer from 1 to 19; (ii) a first gRNA core capable of binding to a Cas9 protein; (iii) a first extension arm comprising: a first primer binding site (PBS) comprising a PBS sequence selected from the same Table as the first spacer, and a first editing template comprising an RTT sequence selected from Table 39, wherein the Paring Spacer number of the RTT sequence in Table 39 is y; and (b) the second PEgRNA comprises: (iv) a second spacer comprising at its 3′ end nucleotides 5-20 of a Spacer sequence selected from Table yA, wherein y is an integer from 20 to 36; (v) a second gRNA core capable of binding to a Cas9 protein; (vi) a second extension arm comprising: a second editing template comprising an RTT sequence selected from Table 40, wherein the Paring Spacer number of the RTT sequence in Table 40 is x, and a second primer binding site (PBS) comprising a PBS sequence selected from the same Table as the second spacer, wherein x in (a) and (b) are the same integer, and wherein y in (a) and (b) are the same integer.

In some embodiments, the first spacer and/or the second spacer is from 16 to 22 nucleotides in length.

In some embodiments, the first spacer and/or the second spacer comprises at its 3′ the selected sequence.

In some embodiments, the first spacer and/or the second spacer is 20 nucleotides in length.

In some embodiments, the first gRNA core and the second gRNA core comprise the same sequence.

In some embodiments, the first gRNA core and the second gRNA core each comprises SEQ ID NO: 3641.

In some embodiments, the first spacer, the first gRNA core, the first editing template, and the first PBS form a contiguous sequence in a single molecule.

In some embodiments, the first pegRNA comprises from 5′ to 3′ the first spacer, the first gRNA core, the first editing template, and the first PBS.

In some embodiments, the second spacer, the second gRNA core, the second editing template, and the second PBS form a contiguous sequence in a single molecule.

In some embodiments, the second pegRNA comprises from 5′ to 3′ the second spacer, the second gRNA core, the second editing template, and the second PBS.

In some embodiments, the prime editing composition of any one of the embodiments described above comprises the first PEgRNA and the second PEgRNA, wherein the first PEgRNA and/or the second PEgRNA further comprises 3′ mN*mN*mN*N and 5′mN*mN*mN*modifications, where m indicates that the nucleotide contains a 2′-O-Me modification and a * indicates the presence of a phosphorothioate bond.

In some embodiments, the prime editing composition of any one of the embodiments described above further comprises a prime editor or one or more polynucleotides encoding the prime editor, wherein the prime editor comprises a Cas9 nickase having a nuclease inactivating mutation in the HNH domain and a reverse transcriptase.

In some embodiments, the Cas9 nickase comprises an amino acid sequence having at least 80%, 85%, 90%, 95%, 96%, 97%, 98%, 99%, or 100% identity to SEQ ID NO: 3582.

In some embodiments, the reverse transcriptase comprises an amino acid sequence having at least 80%, 85%, 90%, 95%, 96%, 97%, 98%, 99%, or 100% identity to SEQ ID NO: 3579.

In some embodiments, the sequence identities are determined by Needleman-Wunsch alignment of two protein sequences with Gap Costs set to Existence: 11 Extension: 1 where percent identity is calculated by dividing the number of identities by the length of the alignment.

In some embodiments, the prime editor is a fusion protein.

In some embodiments, the one or more polynucleotides comprise (a) a first sequence encoding an N-terminal portion of the Cas9 nickase and an intein-N and (b) a second sequence encoding a intein-C, a C-terminal portion of the Cas9 nickase, and the reverse transcriptase.

In some embodiments, the prime editing composition comprises one or more vectors comprising the one or more polynucleotides encoding the first PEgRNA, the one or more polynucleotides encoding the second PEgRNA, and the one or more polynucleotides encoding the prime editor.

In some embodiments, the one or more vectors are AAV vectors.

In some embodiments, provided herein is an LNP comprising the prime editing composition of any one of the above described embodiments.

In an aspect, provided herein is a method of editing a DMPK gene, the method comprising contacting the DMPK gene with (a) the prime editing composition of any one of Embodiments 1-82 and a prime editor comprising a Cas9 nickase having a nuclease inactivation mutation in the HNH domain and a reverse transcriptase, (b) the prime editing composition of any one of Embodiments 83-90, or (c) the LNP of Embodiment 91.

In some embodiments, the DMPK gene is in a cell.

In some embodiments, the cell is a mammalian cell.

In some embodiments, the cell is a human cell.

In some embodiments, the cell is a fibroblast, a myoblast, a myosatellite, a muscle progenitor cell, a cardiomyocyte, a differentiated muscle cell, a skeletal muscle cell, or a smooth muscle cell.

In some embodiments, the cell is in a subject.

In some embodiments, the subject is a human.

In some embodiments, the cell is from a subject having myotonic dystrophy type 1.

In some embodiments, provided herein is a cell generated by any of the methods described above.

In some embodiments, provided herein is a population of cells generated by any of the methods described above.

In an aspect, provided herein is a method for treating myotonic dystrophy type 1 in a subject in need thereof, the method comprising administering to the subject (a) the prime editing composition in the embodiments described above and a prime editor comprising a Cas9 nickase having a nuclease inactivation mutation in the HNH domain and a reverse transcriptase, (b) the prime editing composition the embodiments described above, (c) the LNP, the cell, or the population of cells of the embodiments described above.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 depicts a schematic of a prime editing guide RNA (PEgRNA) binding to a double stranded target DNA sequence.

FIG. 2 depicts a PEgRNA architectural overview in an exemplary schematic of PEgRNA designed for a prime editor.

FIG. 3 is a schematic showing the spacer and gRNA core part of an exemplary guide RNA, in two separate molecules. The rest of the PEgRNA structure is not shown.

FIG. 4A depicts an exemplary schematic of a dual prime editing system for editing both strands of a double stranded target DNA. Same color/shading indicates complementarity or identity between sequences.

FIG. 4B depicts an exemplary schematic of dual prime editing with a replacement duplex (RD) comprising an overlap duplex (OD). Same color/shading indicates complementarity or identity between sequences.

FIG. 4C depicts an exemplary schematic of dual prime editing. Same color/shading indicates complementarity or identity between sequences.

FIG. 4D depicts an exemplary schematic of dual prime editing. Same color/shading indicates complementarity or identity between sequences.

FIG. 4E depicts an exemplary schematic of dual prime editing. Same color/shading indicates complementarity or identity between sequences.

FIG. 4F depicts an exemplary schematic of dual prime editing. Same color/shading indicates complementarity or identity between sequences.

FIG. 4G depicts an exemplary schematic of dual prime editing. Same color/shading indicates complementarity or identity between sequences.

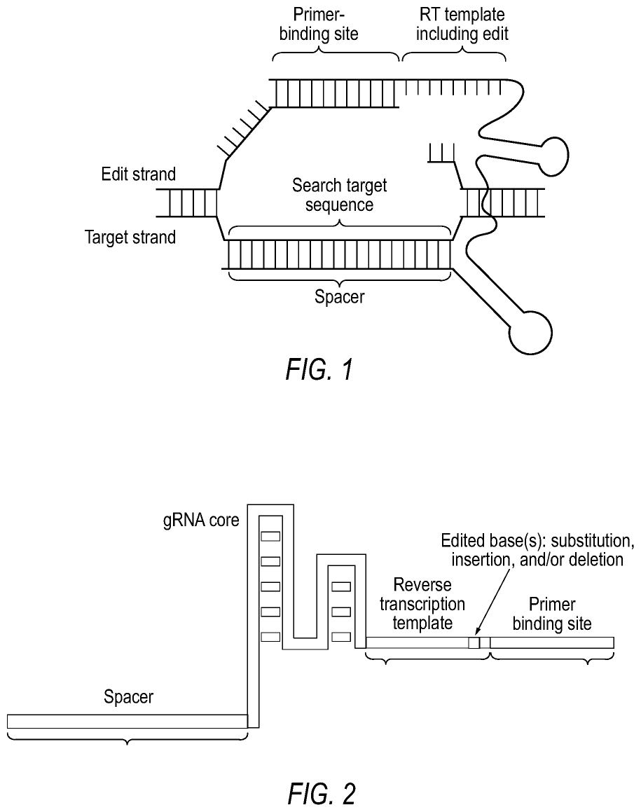

### DETAILED DESCRIPTION OF THE INVENTION

Provided herein, in some embodiments, are compositions and methods to edit the target gene DMPK with dual prime editing. In certain embodiments, provided herein are compositions and methods for correction of mutations in the DMPK gene associated with myotonic dystrophy type 1 (DM1). Compositions provided herein can comprise prime editors (PEs) and engineered guide polynucleotides, e.g., prime editing guide RNAs (PEgRNAs), that can direct PEs to specific DNA targets and can encode DNA edits on the DMPK gene, including correction of disease-causing mutations in the DMPK gene.

The following description and examples illustrate embodiments of the present disclosure in detail. It is to be understood that this disclosure is not limited to the particular embodiments described herein and as such can vary. Those of skill in the art will recognize that there are numerous variations and modifications of this disclosure, which are encompassed within its scope. Although various features of the present disclosure can be described in the context of a single embodiment, the features can also be provided separately or in any suitable combination. Conversely, although the present disclosure can be described herein in the context of separate embodiments for clarity, the present disclosure can also be implemented in a single embodiment.

### Definitions

This disclosure refers to position numbers in polynucleotides. Unless otherwise noted, nucleotide x, in a polynucleotide sequence, refers to the nucleotide at position number x in the polynucleotide sequence from a 5′ to 3′ order.

Unless defined otherwise, all technical and scientific terms used herein have the same meaning as is commonly understood by one of ordinary skill in the art.

The terminology used herein is for the purpose of describing particular embodiments only and is not intended to be limiting. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used herein, they mean “comprising”.

Unless otherwise specified, the words “comprising”, “comprise”, “comprises”, “having”, “have”, “has”, “including”, “includes”, “include”, “containing”, “contains” and “contain” are inclusive or open-ended and do not exclude additional, unrecited elements or method steps.

Reference to “some embodiments”, “an embodiment”, “one embodiment”, or “other embodiments” means that a particular feature or characteristic described in connection with the embodiments is included in at least one or more embodiments, but not necessarily all embodiments, of the present disclosure.

The term “about” or “approximately” in relation to a numerical means, a range of values that fall within 10% greater than or less than the value. For example, about x means x±(10%*x).

The term “between” when used with reference to a range of numbers, means the range of numbers including the first and the last number in the range.

As used herein, a “cell” can generally refer to a biological cell. A cell can be the basic structural, functional and/or biological unit of a living organism. A cell can originate from any organism having one or more cells. Some non-limiting examples include: a prokaryotic cell, eukaryotic cell, a bacterial cell, an archaeal cell, a cell of a single-cell eukaryotic organism, a protozoa cell, a cell from a plant, an animal cell, a cell from an invertebrate animal (e.g. fruit fly, cnidarian, echinoderm, nematode, etc.), a cell from a vertebrate animal (e.g., fish, amphibian, reptile, bird, mammal), a cell from a mammal (e.g., a pig, a cow, a goat, a sheep, a rodent, a rat, a mouse, a non-human primate, a human, etc.), et cetera. Sometimes a cell may not originate from a natural organism (e.g., a cell can be synthetically made, sometimes termed an artificial cell).

In some embodiments, the cell is a human cell. A cell may be of or derived from different tissues, organs, and/or cell types. In some embodiments, the cell is a primary cell. In some embodiments, the term primary cell means a cell isolated from an organism, e.g., a mammal, which is grown in tissue culture (i.e., in vitro) for the first time before subdivision and transfer to a subculture. In some non-limiting examples, mammalian primary cells can be modified through introduction of one or more polynucleotides, polypeptides, and/or prime editing compositions (e.g., through transfection, transduction, electroporation and the like) and further passaged. Such modified mammalian primary cells include muscle cells (e.g., cardiac muscle cells, smooth muscle cells, myosatellite cells), epithelial cells (e.g., mammary epithelial cells, intestinal epithelial cells, hepatocytes), endothelial cells, glial cells, neural cells, formed elements of the blood (e.g., lymphocytes, bone marrow cells), precursors of any of these somatic cell types, and stem cells. In some embodiments, the cell is a fibroblast. In some embodiments, the cell is a human fibroblast. In some embodiments, the cell is a myogenic cell. In some embodiments, the cell is a myoblast. In some embodiments, the cell is a human myogenic cell. In some embodiments, the cell is a human myoblast. In some embodiments, the cell is a primary muscle cell. In some embodiments, the cell is a myosatellite cell (a satellite cell). In some embodiments, the cell is a human myosatellite cell (a satellite cell). In some embodiments, the cell is a stem cell. In some embodiments, the cell is an embryonic stem cell (ESC). In some embodiments, the cell is an induced human pluripotent stem cell (iPSC). In some embodiments, the cell is a human stem cell. In some embodiments, the cell is a human embryonic stem cell.

In some embodiments, a cell is not isolated from an organism but forms part of a tissue or organ of an organism, e.g., a mammal. In some non-limiting examples, mammalian cells include muscle cells (e.g., cardiac muscle cells, smooth muscle cells, myosatellite cells), epithelial cells (e.g., mammary epithelial cells, intestinal epithelial cells, hepatocytes), endothelial cells, glial cells, neural cells, formed elements of the blood (e.g., lymphocytes, bone marrow cells), precursors of any of these somatic cell types, and stem cells. In some embodiments, the cell is a primary muscle cell. In some embodiments, the cell is a myosatellite cell (a satellite cell). In some embodiments, the cell is a human myosatellite cell (a satellite cell). In some embodiments, the cell is a stem cell. In some embodiments, the cell is a human stem cell.

In some embodiments, the cell is a progenitor cell. In some embodiments, the cell is a differentiated cell. In some embodiments, the cell is a fibroblast. In some embodiments, the cell is a myogenic cell. In some embodiments, the cell is a myoblast. In some embodiments, the cell is a human myogenic cell. In some embodiments, the cell is a human myoblast. In some embodiments, the cell is a differentiated muscle cell. In some embodiments, the cell is a myosatellite cell. In some embodiments, the cell is a skeletal muscle cell. In some embodiments, the skeletal muscle cell is differentiated from an iPSC, ESC or myosatellite cell. In some embodiments, the cell is a differentiated human cell. In some embodiments, the cell is a human fibroblast. In some embodiments, the cell is a differentiated human muscle cell. In some embodiments, the cell is a human myosatellite cell. In some embodiments, the cell is a human skeletal muscle cell. In some embodiments, the human skeletal muscle cell is differentiated from a human iPSC, human ESC or human myosatellite cell. In some embodiments, a human myosatellite cell is differentiated from a human iPSC or human ESC.

In some embodiments, the cell comprises a prime editor or a prime editing composition. In some embodiments, the cell comprises a dual prime editing composition comprising a prime editor and at least two PEgRNAs that are different from each other. In some embodiments, the cell is from a human subject. In some embodiments, the human subject has a disease or condition associated with one or more mutations to be corrected by prime editing, for example, myotonic dystrophy type 1 (DM1). In some embodiments, the cell is from a human subject, and comprises a prime editor or a prime editing composition for correction of the one or more mutations. In some embodiments, the cell is from the human subject and the mutation has been edited or corrected by prime editing. In some embodiments, the cell is in a human subject, and comprises a prime editor or a prime editing composition for correction of the one or more mutations. In some embodiments, the cell is from the human subject and the mutation has been edited or corrected by prime editing.

The term “substantially” as used herein may refer to a value approaching 100% of a given value. In some embodiments, the term may refer to an amount that may be at least about 70%, 80%, 90%, 91%, 92%, 93%, 94%, 95%, 96%, 97%, 98%, 99%, 99.9%, or 99.99% of a total amount. In some embodiments, the term may refer to an amount that may be about 100% of a total amount.

The terms “protein” and “polypeptide” can be used interchangeably to refer to a polymer of two or more amino acids joined by covalent bonds (e.g., an amide bond) that can adopt a three-dimensional conformation. In some embodiments, a protein or polypeptide comprises at least 10 amino acids, 15 amino acids, 20 amino acids, 30 amino acids or 50 amino acids joined by covalent bonds (e.g., amide bonds). In some embodiments, a protein comprises at least two amide bonds. In some embodiments, a protein comprises multiple amide bonds. In some embodiments, a protein comprises an enzyme, enzyme precursor proteins, regulatory protein, structural protein, receptor, nucleic acid binding protein, a biomarker, a member of a specific binding pair (e.g., a ligand or aptamer), or an antibody. In some embodiments, a protein may be a full-length protein (e.g., a fully processed protein having certain biological function). In some embodiments, a protein may be a variant or a fragment of a full-length protein. For example, in some embodiments, a Cas9 protein domain comprises an H840A amino acid substitution compared to a naturally occurring S. pyogenes Cas9 protein. A variant of a protein or enzyme, for example a variant reverse transcriptase, comprises a polypeptide having an amino acid sequence that is about 60% identical, about 70% identical, about 80% identical, about 90% identical, about 95% identical, about 96% identical, about 97% identical, about 98% identical, about 99% identical, about 99.5% identical, or about 99.9% identical to the amino acid sequence of a reference protein.

In some embodiments, a protein comprises one or more protein domains or subdomains. As used herein, the term “polypeptide domain”, “protein domain”, or “domain” when used in the context of a protein or polypeptide, refers to a polypeptide chain that has one or more biological functions, e.g., a catalytic function, a protein-protein binding function, or a protein-DNA function. In some embodiments, a protein comprises multiple protein domains. In some embodiments, a protein comprises multiple protein domains that are naturally occurring. In some embodiments, a protein comprises multiple protein domains from different naturally occurring proteins. For example, in some embodiments, a prime editor may be a fusion protein comprising a Cas9 protein domain of S. pyogenes and a reverse transcriptase protein domain of Moloney murine leukemia virus. A protein that comprises amino acid sequences from different origins or naturally occurring proteins may be referred to as a fusion, or chimeric protein.

In some embodiments, a protein comprises a functional variant or functional fragment of a full-length wild type protein. A “functional fragment” or “functional portion”, as used herein, refers to any portion of a reference protein (e.g., a wild type protein) that encompasses less than the entire amino acid sequence of the reference protein while retaining one or more of the functions, e.g., catalytic or binding functions. For example, a functional fragment of a reverse transcriptase may encompass less than the entire amino acid sequence of a wild type reverse transcriptase, but retains the ability under at least one set of conditions to catalyze the polymerization of a polynucleotide. When the reference protein is a fusion of multiple functional domains, a functional fragment thereof may retain one or more of the functions of at least one of the functional domains. For example, a functional fragment of a Cas9 may encompass less than the entire amino acid sequence of a wild type Cas9, but retains its DNA binding ability and lacks its nuclease activity partially or completely.

A “functional variant” or “functional mutant”, as used herein, refers to any variant or mutant of a reference protein (e.g., a wild type protein) that encompasses one or more alterations to the amino acid sequence of the reference protein while retaining one or more of the functions, e.g., catalytic or binding functions. In some embodiments, the one or more alterations to the amino acid sequence comprises amino acid substitutions, insertions or deletions, or any combination thereof. In some embodiments, the one or more alterations to the amino acid sequence comprises amino acid substitutions. For example, a functional variant of a reverse transcriptase may comprise one or more amino acid substitutions compared to the amino acid sequence of a wild type reverse transcriptase, but retains the ability under at least one set of conditions to catalyze the polymerization of a polynucleotide. When the reference protein is a fusion of multiple functional domains, a functional variant thereof may retain one or more of the functions of at least one of the functional domains. For example, in some embodiments, a functional variant of a Cas9 may comprise one or more amino acid substitutions in a nuclease domain, e.g., an H840A amino acid substitution, compared to the amino acid sequence of a wild type Cas9, but retains the DNA binding ability and lacks the nuclease activity partially or completely.

The term “function” and its grammatical equivalents as used herein may refer to a capability of operating, having, or serving an intended purpose. Functional may comprise any percent from baseline to 100% of an intended purpose. For example, functional may comprise or comprise about 5%, 10%, 15%, 20%, 25%, 30%, 35%, 40%, 45%, 50%, 55%, 60%, 65%, 70%, 75%, 80%, 85%, 90%, 95%, or up to about 100% of an intended purpose. In some embodiments, the term functional may mean over or over about 100% of normal function, for example, 125%, 150%, 175%, 200%, 250%, 300%, 400%, 500%, 600%, 700% or up to about 1000% of an intended purpose.

In some embodiments, a protein or polypeptide includes naturally occurring amino acids (e.g., one of the twenty amino acids commonly found in peptides synthesized in nature, and known by the one letter abbreviations A, R, N, C, D, Q, E, G, H, I, L, K, M, F, P, S, T, W, Y and V). In some embodiments, a protein or polypeptides includes non-naturally occurring amino acids (e.g., amino acids which is not one of the twenty amino acids commonly found in peptides synthesized in nature, including synthetic amino acids, amino acid analogs, and amino acid mimetics). In some embodiments, a protein or polypeptide is modified.

In some embodiments, a protein comprises an isolated polypeptide. The term “isolated” means free or removed to varying degrees from components which normally accompany it as found in the natural state or environment. For example, a polypeptide naturally present in a living animal is not isolated, and the same polypeptide partially or completely separated from the coexisting materials of its natural state is isolated.

In some embodiments, a protein is present within a cell, a tissue, an organ, or a virus particle. In some embodiments, a protein is present within a cell or a part of a cell (e.g., a bacteria cell, a plant cell, or an animal cell). In some embodiments, the cell is in a tissue, in a subject, or in a cell culture. In some embodiments, the cell is a microorganism (e.g., a bacterium, fungus, protozoan, or virus). In some embodiments, a protein is present in a mixture of analytes (e.g., a lysate). In some embodiments, the protein is present in a lysate from a plurality of cells or from a lysate of a single cell.

The terms “homologous,” “homology,” or “percent homology” as used herein refer to the degree of sequence identity between an amino acid or polynucleotide sequence and a corresponding reference sequence. “Homology” can refer to polymeric sequences, e.g., polypeptide or DNA sequences that are similar. Homology can mean, for example, nucleic acid sequences with at least about: 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identity. In other embodiments, a “homologous sequence” of nucleic acid sequences may exhibit 93%, 95% or 98% sequence identity to the reference nucleic acid sequence. For example, a “region of homology to a genomic region” can be a region of DNA that has a similar sequence to a given genomic region in the genome. A region of homology can be of any length that is sufficient to promote stable binding of a spacer, primer binding site or protospacer sequence to the complementary sequence of a genomic region. For example, the region of homology can comprise at least 5, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100 or more bases in length such that the region of homology has sufficient homology to undergo binding to the complementary sequence of a corresponding genomic region.

When a percentage of sequence homology or identity is specified, in the context of two nucleic acid sequences or two polypeptide sequences, the percentage of homology or identity generally refers to the alignment of two or more sequences across a portion of their length when compared and aligned for maximum correspondence. When a position in the compared sequence can be occupied by the same base or amino acid, then the molecules can be homologous at that position. Unless stated otherwise, sequence homology or identity is assessed over the specified length of the nucleic acid, polypeptide or portion thereof. In some embodiments, the homology or identity is assessed over a functional portion or specified portion of the length.

Alignment of sequences for assessment of sequence homology can be conducted by algorithms known in the art, such as the Basic Local Alignment Search Tool (BLAST) algorithm, which is described in Altschul et al, J. Mol. Biol. 215:403-410, 1990. A publicly available, internet interface, for performing BLAST analyses is accessible through the National Center for Biotechnology Information. Additional known algorithms include those published in: Smith & Waterman, “Comparison of Biosequences”, Adv. Appl. Math. 2:482, 1981; Needleman & Wunsch, “A general method applicable to the search for similarities in the amino acid sequence of two proteins” J. Mol. Biol. 48:443, 1970; Pearson & Lipman “Improved tools for biological sequence comparison”, Proc. Natl. Acad. Sci. USA 85:2444, 1988; or by automated implementation of these or similar algorithms. Global alignment programs may also be used to align similar sequences of roughly equal size. Examples of global alignment programs include NEEDLE (available at www.ebi.ac.uk/Tools/psa/emboss_needle/) which is part of the EMBOSS package (Rice P et al., Trends Genet., 2000; 16: 276-277), and the GGSEARCH program https://fasta.bioch.virginia.edu/fasta_www2/, which is part of the FASTA package (Pearson W and Lipman D, 1988, Proc. Natl. Acad. Sci. USA, 85: 2444-2448). Both of these programs are based on the Needleman-Wunsch algorithm which is used to find the optimum alignment (including gaps) of two sequences along their entire length. A detailed discussion of sequence analysis can also be found in Unit 19.3 of Ausubel et al (“Current Protocols in Molecular Biology” John Wiley & Sons Inc, 1994-1998, Chapter 15, 1998). In some embodiments, alignment between a query sequence and a reference sequence is performed with Needleman-Wunsch alignment with Gap Costs set to Existence: 11 Extension: 1 where percent identity is calculated by dividing the number of identities by the length of the alignment, as further described in Altschul et al. (“Gapped BLAST and PSI-BLAST: a new generation of protein database search programs”, Nucleic Acids Res. 25:3389-3402, 1997) and Altschul et al, (“Protein database searches using compositionally adjusted substitution matrices”, FEBS J. 272:5101-5109, 2005).

A skilled person understands that amino acid (or nucleotide) positions may be determined in homologous sequences based on alignment, for example, “H840” in a reference Cas9 sequence may correspond to H839, or another position in a Cas9 homolog.

The term “polynucleotide” or “nucleic acid molecule” can be any polymeric form of nucleotides, including DNA, RNA, a hybridization thereof, or RNA-DNA chimeric molecules. In some embodiments, a polynucleotide comprises cDNA, genomic DNA, mRNA, tRNA, rRNA, or microRNA. In some embodiments, a polynucleotide is double stranded, e.g., a double-stranded DNA in a gene. In some embodiments, a polynucleotide is single-stranded or substantially single-stranded, e.g., single-stranded DNA or an mRNA. In some embodiments, a polynucleotide is a cell-free nucleic acid molecule. In some embodiments, a polynucleotide circulates in blood. In some embodiments, a polynucleotide is a cellular nucleic acid molecule. In some embodiments, a polynucleotide is a cellular nucleic acid molecule in a cell circulating in blood.

Polynucleotides can have any three-dimensional structure. The following are nonlimiting examples of polynucleotides: a gene or gene fragment (for example, a probe, primer, EST or SAGE tag), an exon, an intron, intergenic DNA (including, without limitation, heterochromatic DNA), messenger RNA (mRNA), transfer RNA (tRNA), ribosomal RNA (rRNA), a ribozyme, cDNA, a recombinant polynucleotide, a branched polynucleotide, a plasmid, a vector, isolated DNA, isolated RNA, sgRNA, guide RNA, a nucleic acid probe, a primer, an snRNA, a long non-coding RNA, a snoRNA, a siRNA, a miRNA, a tRNA-derived small RNA (tsRNA), an antisense RNA, an shRNA, or a small rDNA-derived RNA (srRNA).

In some embodiments, a polynucleotide comprises deoxyribonucleotides, ribonucleotides or analogs thereof. In some embodiments, a polynucleotide comprises modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component.

In some embodiments, a polynucleotide is composed of a specific sequence of four nucleotide bases: adenine (A); cytosine (C); guanine (G); thymine (T); and uracil (U) for thymine when the polynucleotide is RNA. In some embodiments, the polynucleotide may comprise one or more other nucleotide bases, such as inosine (I), which is read by the translation machinery as guanine (G).

In some embodiments, a polynucleotide may be modified. As used herein, the terms “modified” or “modification” refers to chemical modification with respect to the A, C, G, T and U nucleotides. In some embodiments, modifications may be on the nucleoside base and/or sugar portion of the nucleosides that comprise the polynucleotide. In some embodiments, the modification may be on the internucleoside linkage (e.g., phosphate backbone). In some embodiments, multiple modifications are included in the modified nucleic acid molecule. In some embodiments, a single modification is included in the modified nucleic acid molecule.

The term “complement”, “complementary”, or “complementarity”, as used herein, refers to the ability of two polynucleotide molecules to base pair with each other. Complementary polynucleotides may base pair via hydrogen bonding, which may be Watson Crick, Hoogsteen or reversed Hoogsteen hydrogen bonding. For example, an adenine on one polynucleotide molecule will base pair to a thymine or uracil on a second polynucleotide molecule and a cytosine on one polynucleotide molecule will base pair to a guanine on a second polynucleotide molecule. Two polynucleotide molecules are complementary to each other when a first polynucleotide molecule comprising a first nucleotide sequence can base pair with a second polynucleotide molecule comprising a second nucleotide sequence. For instance, the two DNA molecules 5′-ATGC-3′ and 5′-GCAT-3′ are complementary, and the complement of the DNA molecule 5′-ATGC-3′ is 5′-GCAT-3′. A percentage of complementarity indicates the percentage of nucleotides in a polynucleotide molecule which can base pair with a second polynucleotide molecule (e.g., 5, 6, 7, 8, 9, 10 out of 10 being 50%, 60%, 70%, 80%, 90%, and 100% complementary, respectively). “Perfectly complementary” means that all the contiguous nucleotides of a polynucleotide molecule will base pair with the same number of contiguous nucleotides in a second polynucleotide molecule. “Substantially complementary” as used herein refers to a degree of complementarity that can be 70%, 75%, 80%, 85%, 90%, 95%, 97%, 98%, or 99% over all or a portion of two polynucleotide molecules. In some embodiments, the portion of complementarity may be a region of 10, 15, 20, 25, 30, 35, 40, 45, 50, or more nucleotides. In some embodiments, the portion of complementarity between the two polynucleotide molecules is at least 60%, 65%, 70%, 75%, 80%, 85%, 90%, 95%, 97%, 98%, or 99% of the length of at least one of the two polynucleotide molecules or a functional or defined portion thereof.

As used herein, “expression” refers to the process by which polynucleotides are transcribed into mRNA and/or the process by which polynucleotides, e.g., the transcribed mRNA, are translated into peptides, polypeptides, or proteins. If the polynucleotide is derived from genomic DNA, expression may include splicing of the mRNA in a eukaryotic cell. In some embodiments, expression of a polynucleotide, e.g., a gene or a DNA encoding a protein, is determined by the amount of the protein encoded by the gene after transcription and translation of the gene. In some embodiments, expression of a polynucleotide, e.g., a gene or a DNA encoding a protein, is determined by the amount of a functional form of the protein encoded by the gene after transcription and translation of the gene. In some embodiments, expression of a gene is determined by the amount of the mRNA, or transcript, that is encoded by the gene after transcription of the gene. In some embodiments, expression of a polynucleotide, e.g., an mRNA, is determined by the amount of the protein encoded by the mRNA after translation of the mRNA. In some embodiments, expression of a polynucleotide, e.g., a mRNA or coding RNA, is determined by the amount of a functional form of the protein encoded by the polypeptide after translation of the polynucleotide.

The terms “equivalent” or “biological equivalent” are used interchangeably when referring to a particular molecule, or biological or cellular material, and means a molecule having minimal homology to another molecule while still maintaining a desired structure or functionality.

The term “encode” as it is applied to polynucleotides refers to a nucleic acid, in its native state or when manipulated by methods well known to those skilled in the art, contains the information that can be used as a template for synthesis of another nucleic acid, amino acid, or polypeptide. For example, a DNA sequence can encode a RNA sequence and can serve as the template for transcription of the RNA sequence. A RNA sequence can encode a DNA sequence and can serve as the template for reverse transcription of the DNA sequence. A DNA or RNA sequence can encode a polypeptide sequence and can contain the information for the translation template of the polypeptide sequence. In some embodiments, one or more or a few nucleotides can be said to specifically encode a mutation or a desired nucleotide edit. In some embodiments, a polynucleotide comprising three contiguous nucleotides form a codon that encodes a specific amino acid. In some embodiments, a polynucleotide comprises one or more codons that encode a polypeptide. In some embodiments, a polynucleotide comprising one or more codons comprises a mutation in a codon compared to a wild-type reference polynucleotide. In some embodiments, the mutation in the codon encodes an amino acid substitution in a polypeptide encoded by the polynucleotide as compared to a wild-type reference polypeptide.

The term “mutation” as used herein refers to a change and/or alteration in an amino acid sequence of a protein or nucleic acid sequence of a polynucleotide. Such changes and/or alterations may comprise the substitution, insertion, deletion and/or truncation of one or more amino acids, in the case of an amino acid sequence, and/or nucleotides, in the case of nucleic acid sequence, compared to a reference amino acid or nucleic acid sequence. A mutation in a polynucleotide may be insertion or expansion of one or more nucleotides, or example, expansion of three nucleotides (tri-nucleotide expansion). In some embodiments, the reference sequence is a wild-type sequence. In some embodiments, a mutation in a nucleic acid sequence of a polynucleotide encodes a mutation in the amino acid sequence of a polypeptide. In some embodiments, the mutation in the amino acid sequence of a polypeptide or the mutation in the nucleic acid sequence of a polynucleotide is a mutation associated with a disease state.

The term “subject” and its grammatical equivalents as used herein may refer to a human or a non-human. A subject may be a mammal. A human subject may be male or female. A human subject may be of any age. A subject may be a human embryo. A human subject may be a newborn, an infant, a child, an adolescent, or an adult. A human subject may be in need of treatment for a genetic disease or disorder.

The terms “treatment” or “treating” and their grammatical equivalents may refer to the medical management of a subject with an intent to cure, ameliorate, or ameliorate a symptom of, a disease, condition, or disorder. Treatment may include active treatment, that is, treatment directed specifically toward the improvement of a disease, condition, or disorder. Treatment may include causal treatment, that is, treatment directed toward removal of the cause of the associated disease, condition, or disorder. In addition, this treatment may include palliative treatment, that is, treatment designed for the relief of symptoms rather than the curing of the disease, condition, or disorder. Treatment may include supportive treatment, that is, treatment employed to supplement another specific therapy directed toward the improvement of the disease, condition, or disorder. In some embodiments, a condition may be pathological. In some embodiments, a treatment may not completely cure or prevent a disease, condition, or disorder. In some embodiments, a treatment ameliorates, but does not completely cure or prevent a disease, condition, or disorder. In some embodiments, a subject may be treated for 12 hours, 24 hours, 2 days, 3 days, 4 days, 5 days, 6 days, 7 days, 2 weeks, 3 weeks, 4 weeks, 2 months, 3 months, 4 months, 5 months, 6 months, 1 year, 2 years, 3 years, 4 years, 5 years, 6 years, indefinitely, or life of the subject.

The term “ameliorate” and its grammatical equivalents means to decrease, suppress, attenuate, diminish, arrest, or stabilize the development or progression of a disease.

The terms “prevent” or “preventing” means delaying, forestalling, or avoiding the onset or development of a disease, condition, or disorder for a period of time. Prevent also means reducing risk of developing a disease, disorder, or condition. Prevention includes minimizing or partially or completely inhibiting the development of a disease, condition, or disorder. In some embodiments, a composition, e.g., a pharmaceutical composition, prevents a disorder by delaying the onset of the disorder for 12 hours, 24 hours, 2 days, 3 days, 4 days, 5 days, 6 days, 7 days, 2 weeks, 3 weeks, 4 weeks, 2 months, 3 months, 4 months, 5 months, 6 months, 1 year, 2 years, 3 years, 4 years, 5 years, 6 years, indefinitely, or life of a subject.

The term “effective” means having the ability to produce a biological response. For example, “effective amount” or “therapeutically effective amount” may refer to a quantity of a composition, for example a composition comprising a construct, that can be sufficient to result in a desired activity upon introduction into a subject as disclosed herein. An effective amount of the prime editing compositions can be provided to the target gene or cell, whether the cell is in vitro, ex vivo or in vivo.

The amount of target gene modulation may be measured by any suitable method known in the art. In some embodiments, the “effective amount” or “therapeutically effective amount” is the amount of a composition that is required to ameliorate the symptoms of a disease relative to an untreated patient. In some embodiments, an effective amount is the amount of a composition sufficient to introduce an alteration in a gene of interest in a cell (e.g., a cell in vitro, ex vivo or in vivo).

An effective amount can be the amount to induce, when administered to a population of cells, at least about a 2-fold decrease in the number of cells that have expanded CTG repeat number in the DMPK gene. An effective amount can be the amount to induce, when administered to a population of cells, at least about a 2-fold decrease in the number of cells that have 35 or more CTG repeats in a DMPK mRNA encoded by the DMPK gene.

In some embodiments, an effective amount can be the amount to induce, for example, at least about 1.1-fold decrease, about 1.5 fold decrease, about 2-fold decrease, about 3-fold decrease, about 4-fold decrease, about 5-fold decrease, about 6-fold decrease, about 7-fold decrease, about 8-fold decrease, about 9-fold decrease, about 10-fold decrease, about 25-fold decrease, about 50-fold decrease, about 100-fold decrease, about 200-fold decrease, about 500-fold decrease, about 700-fold decrease, about 1000-fold decrease, about 5000-fold decrease, or about 10,000-fold decrease in the amount of DMPK transcripts having 35 or more CTG repeats.

The term “construct” refers to a polynucleotide or a portion of a polynucleotide, comprising one or more nucleic acid sequences encoding one or more transcriptional products and/or proteins. A construct may be a recombinant nucleic acid molecule or a part thereof. In some embodiments, the one or more nucleic acid sequences of a construct are operably linked to one or more regulatory sequences, for example, transcriptional initiation regulatory sequences. In some embodiments, a construct is a vector, a plasmid, or a portion thereof. In some embodiments, a construct comprises DNA. In some embodiments, a construct comprises RNA. In some embodiments, a construct is double stranded. In some embodiments, a construct is single stranded. In some embodiments, a construct comprises an expression cassette. An expression cassette means a polynucleotide comprising a nucleic acid sequence that encodes one or more transcriptional products and is operably linked to at least one transcriptional regulatory sequence, e.g., a promoter.

The term “exogenous” when used in reference to a biomolecule, e.g., a polynucleotide sequence or a polypeptide sequence refers to a biomolecule that is not native to a specific biological context, e.g., a gene, a particular chromosome, a particular cell or chromosomal site of the cell, tissue, or organism, or, if from the same source, is modified from its original form or is present in a non-native location, e.g., a chromosome location.

The term “endogenous” when used in reference to a biomolecule, e.g., a polynucleotide sequence or a polypeptide sequence refers to a biomolecule that is native to or naturally occurring in a specific biological context, e.g., a gene, a particular chromosome, a particular cell or chromosomal site of the cell, tissue, or organism. For example, an endogenous sequence may be a wild type sequence or may comprise one or more mutations compared to a wild type sequence. In some embodiments, an endogenous sequence is mutated compared to a wild type sequence and may cause or be associated with a disease or disorder in a subject. As used herein, in some embodiments, a wild type sequence, with respect to a specific gene and a specific disease, is a gene sequence found in healthy individuals, wherein the wild-type sequence does not include a mutation causative of the specific disease. In some embodiments, in the context of repeat expansion disease, a wild type sequence may be used to refer to a sequence that harbors an array of a specific tri-nucleotide repeats within a normal range. For example, in some embodiments, the tri-nucleotide repeat is a CTG repeat in a DMPK gene, and a wild type sequence of a DMPK gene may have 5 to 34 CTG repeats.

A “repeat expansion disorder” or “trinucleotide repeat disorder” or “expansion repeat disorder” refers to a set of genetic disorders which are caused by “trinucleotide repeat expansion,” which is a kind of mutation characterized by expanded number of repeats of a contiguous array of three nucleotides each having the same sequence, referred to as “trinucleotide repeats” or “triplet repeats”). As used herein, an array of tri-nucleotide repeats means at least two contiguous tri-nucleotides that are the same. In some embodiments, an array of tri-nucleotide repeats comprises 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 repeats of the same tri-nucleotides. In some embodiments, an array of tri-nucleotide repeats comprises at least 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, or 1500 repeats of the same tri-nucleotides. In some embodiments, an array of tri-nucleotide repeats comprises about 50 to 100, about 100 to 150, about 150 to 200, about 200 to 250, about 250 to 300, about 300 to 400, about 400 to 500, about 500 to 600, about 600 to 700, about 700 to 800, about 800 to 900, about 900 to 1000 repeats of the same tri-nucleotides. In some embodiments, an array of tri-nucleotide repeats comprises more than 1000 repeats of the same tri-nucleotides. In some embodiments, an array of tri-nucleotide repeats comprises about 5 to 10, 5 to 15, 5 to 20, 5 to 25, 5 to 30, 5 to 35, 5 to 40, 5 to 45, 5 to 50, 10 to 15, 10 to 20, 10 to 25, 10 to 30, 10 to 35, 10 to 40, 10 to 45, 10 to 50, 15 to 20, 15 to 25, 15 to 30, 15 to 35, 15 to 40, 15 to 45, 15 to 50, 20 to 25, 20 to 30, 20 to 35, 20 to 40, 20 to 45, 20 to 50, 25 to 30, 25 to 35, 25 to 40, 25 to 45, 25 to 50, 30 to 35, 30 to 40, 30 to 45, 30 to 50, 35 to 40, 35 to 45, 35 to 50, 40 to 45, 40 to 50, or 45 to 50 repeats of the same tri-nucleotides. In some embodiments, an array of tri-nucleotide repeats is in a non-coding region of a gene, for example, a 3′ UTR, a 5′ UTR, or an intron of a gene. In some embodiments, an array of tri-nucleotide repeats is in a regulatory sequence, e.g., a promoter, of a gene. In some embodiments, an array of tri-nucleotide repeats is in an upstream sequence or a downstream sequence of a gene. In some embodiments, an array of tri-nucleotide repeats is in a coding region of a gene. In some embodiments, an array of tri-nucleotide repeats encodes an array of amino acid repeats. In some embodiments, the number of repeats of the same tri-nucleotides in an array of tri-nucleotide repeats is altered as compared to the number of repeats in a reference array of tri-nucleotide repeats, for example, the number of repeats in a wild type gene sequence. In some embodiments, the number of repeats of the same tri-nucleotides in an array of tri-nucleotide repeats is increased as compared to the number of repeats in a reference array of tri-nucleotide repeats, for example, the number of repeats in a wild type gene sequence. In some embodiments, the altered or increased number of repeats in an array of tri-nucleotide repeats compared to the number of the same repeats in a wild type gene sequence is associated with a disease.

### Dual Prime Editing

In some embodiments, prime editing may comprise programmable editing of a target DNA using one or more prime editors each complexed with a PEgRNA (“dual prime editing”). Dual prime editing refers to programmable editing of a double stranded target DNA using two or more PEgRNAs, each of which is complexed with a prime editor for incorporating one or more intended nucleotide edits into the double stranded target DNA. In some embodiments, dual prime editing incorporates one or more intended nucleotide edits into a double stranded target DNA through excision of an endogenous DNA segment and/or replacement of the endogenous DNA segment with newly synthesized DNA via target-primed DNA synthesis. In some embodiments, dual prime editing may be used to edit a target DNA that is or is part of a target gene. In some embodiments, the target gene is a disease-associated gene. In some embodiments, the target gene is a monogenic disease-associated gene. In some embodiments, the target gene is a polygenic disease-associated gene. In some embodiments, the target gene is mutated compared to a wild-type sequence of the same gene and may cause or be associated with a disease or disorder in a subject. In some embodiments, the mutated target gene causes a disease or a disorder in a human subject.

The term “prime editing” refers to programmable editing of a target DNA using a prime editor complexed with a PEgRNA to incorporate an intended nucleotide edit into the target DNA through target-primed DNA synthesis. In prime editing, a target DNA may comprise a double stranded DNA molecule having two complementary strands. When viewed in the context of each specific PEgRNA, the two complementary strands of a double stranded target DNA may comprise a strand that may be referred to as a “target strand” or a “non-edit strand”, and a complementary strand that may be referred to as a “non-target strand,” or an “edit strand.” In some embodiments, in a prime editing guide RNA (PEgRNA), a spacer sequence is complementary or substantially complementary to a specific sequence on the target strand, which may be referred to as a “search target sequence”. In some embodiments, the spacer sequence anneals with the target strand at the search target sequence. The target strand may also be referred to as the “non-Protospacer Adjacent Motif (non-PAM strand).” In some embodiments, the non-target strand may also be referred to as the “PAM strand”. In some embodiments, the PAM strand comprises a protospacer sequence and optionally a PAM sequence. A protospacer sequence refers to a specific sequence in the PAM strand of the target gene that is complementary to the search target sequence. In a PEgRNA, a spacer sequence may have a substantially identical sequence as the protospacer sequence on the edit strand of a target gene, except that the spacer sequence may comprise Uracil (U) and the protospacer sequence may comprise Thymine (T).

In some embodiments, dual prime editing involves using two different PEgRNAs each complexed with a prime editor, wherein each of the two PEgRNAs comprises a spacer complementary or substantially complementary to a separate search target sequence. In some embodiments, each of the two PEgRNAs anneals with a separate search target sequence through its spacer. Accordingly, references to a “PAM strand”, a “non-PAM strand”, a “target strand’, a “non-target strand”, an “edit strand” or a “non-edit strand” are relative in the context of a specific PEgRNA, e.g., one of the two PEgRNAs in dual prime editing.

In some embodiments, dual prime editing involves two PEgRNAs, different from one another, each complexed with a prime editor. In some embodiments, each of the two PEgRNAs comprises a region of complementarity to a distinct search target sequence of the target DNA, wherein the two distinct search target sequences are on the two complementary strands of the target DNA. The terms “region”, “portion”, and “segment” are used interchangeably to refer to a proportion of a molecule, for example, a polynucleotide or a polypeptide. For example, a region of a polynucleotide may be 5%, 10%, 15%, 20%, 25%, 30%, 35%, 40%, 45%, 50%, 55%, 60%, 65%, 70%, 75%, 80%, 85%, 90%, 95% or 100% of the polynucleotide. In some embodiments, the two PEgRNAs each can direct a prime editor to initiate the prime editing process on the two complementary strands of the target DNA.

In some embodiments, dual prime editing involves two PEgRNAs each complexed with a prime editor. In some embodiments, a first PEgRNA comprises a first spacer complementary to a first search target sequence on a first strand of a double stranded target DNA, e.g., a double stranded target gene. In the context of the first PEgRNA, the first strand of the double stranded target DNA may be referred to as a first target strand, and the complementary strand referred to as the first PAM strand.

In some embodiments, a second PEgRNA comprises a second spacer complementary to a second search target sequence on a second strand of the double stranded target DNA. In some embodiments, the first strand and the second strand of the double stranded target DNA, e.g., a double stranded target gene, are complementary to each other. Accordingly, in some embodiments, the second PEgRNA and the first PEgRNA bind opposite strands of the double stranded target DNA. In the context of the second PEgRNA, the second strand of the double stranded target DNA may be referred to as a second target strand, and the complementary strand referred to as the second PAM strand. In some embodiments, the first target strand is the same strand as the second PAM strand of the double stranded target DNA. In some embodiments, the second target strand is the same strand as the first PAM strand of the double stranded target DNA.

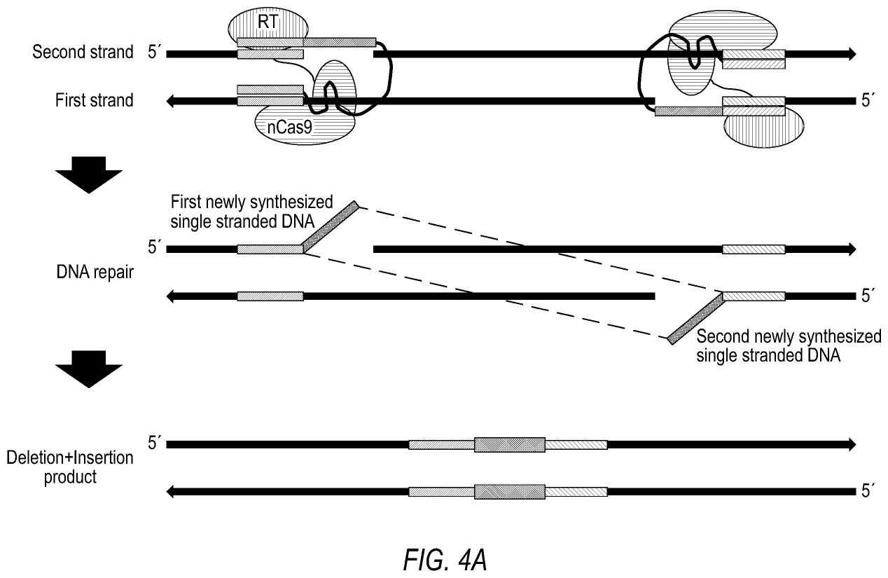

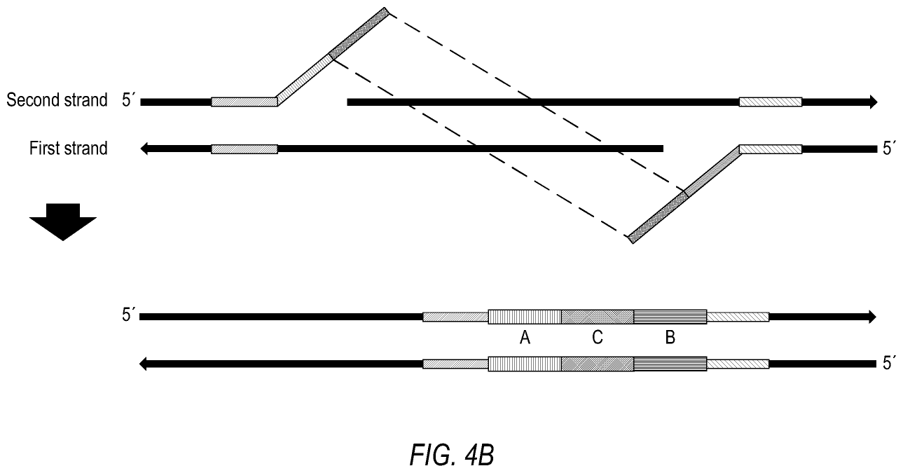

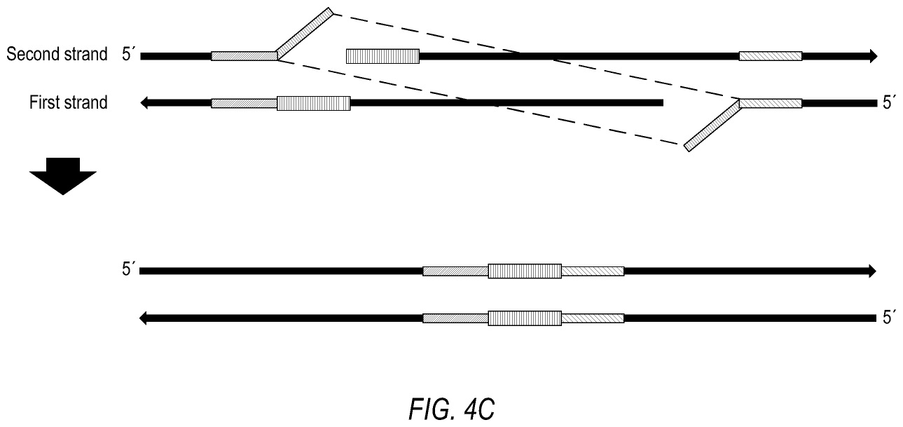

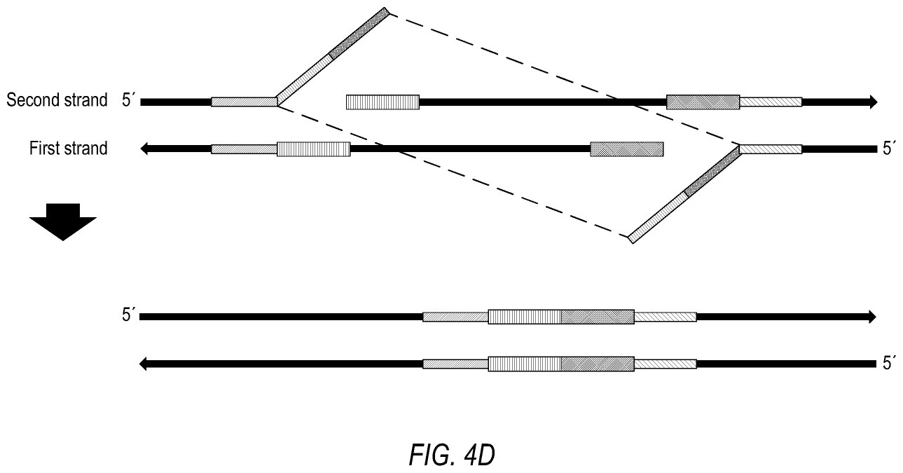

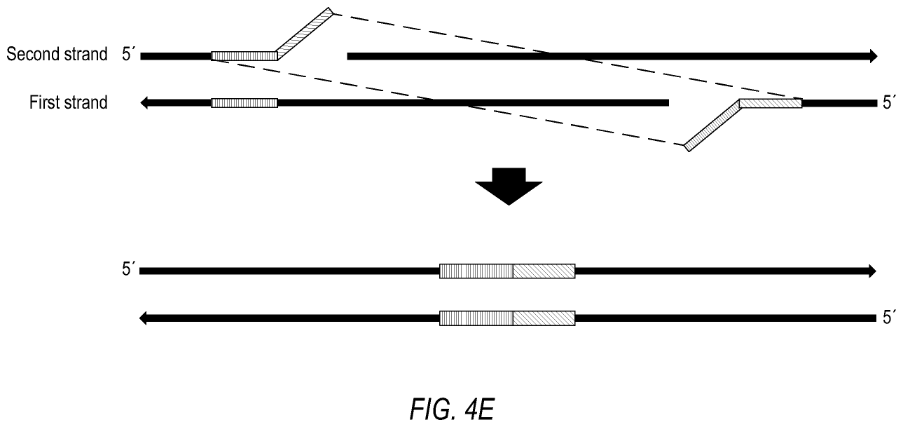

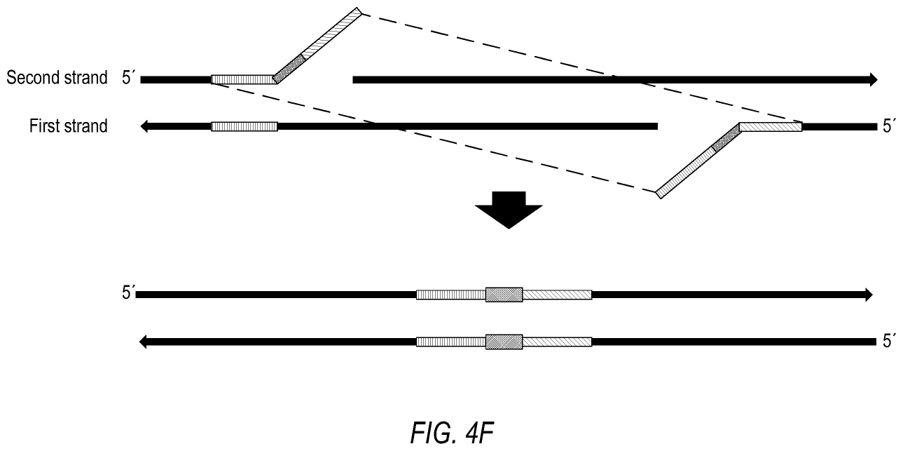

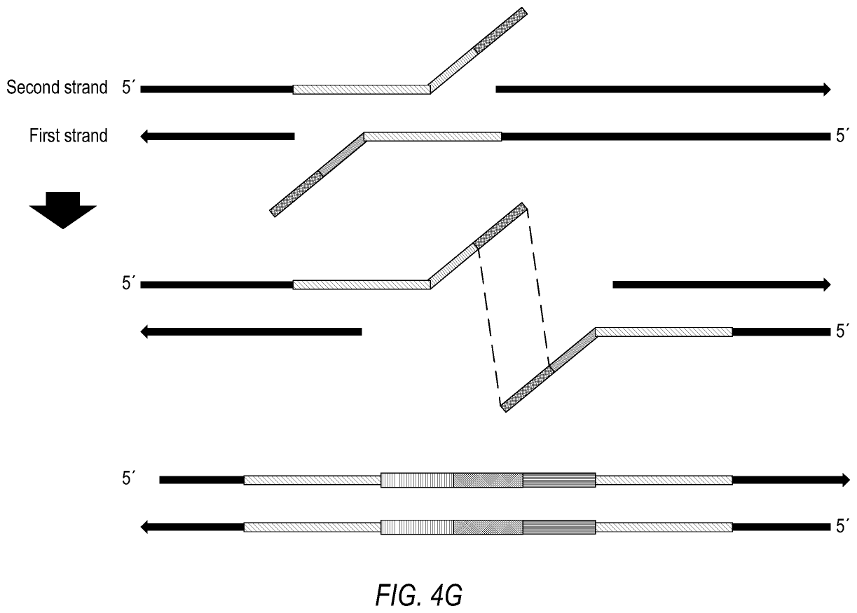

As used herein for editing of the DMPK gene, the first PEgRNA may also be referred to as the “5′ PEgRNA”, and the second PEgRNA may be referred to as the “3′ PEgRNA”. Specifically, the 5′ to 3′ orientation of the DMPK gene refers to the 5′ to 3′ orientation of the coding strand (i.e. sense strand) of the DMPK gene. The first PEgRNA (5′ PEgRNA) comprises a first spacer having complementarity to a first search target sequence on the non-coding strand of DMPK, and is capable of directing a prime editor to nick the coding strand at a first nick site that is 5′ to the CTG repeats. The second PEgRNA (3′ PEgRNA) comprises a second spacer having complementarity to a second search target sequence on the coding strand of DMPK, and is capable of directing a prime editor to nick the non-coding strand at a second nick site that is 5′ to the CAG repeats (that is, the position corresponding to the second nick site on the coding strand is 3′ to the CTG repeats). An exemplary dual prime editing strategy for editing the DMPK gene is provided in FIG. 4A, where the first strand (bottom) is the non-coding strand, and the second strand (top) is the coding strand. The (CTG) repeats are accordingly in the coding strand, and the non-coding strand contains the complementary (CAG) repeats. A 5′ PEgRNA complexed with a prime editor is at the left side of the figure, and a 3′ PEgRNA complexed with a prime editor is at the right side.

In some embodiments, the first PEgRNA anneals with the first target strand of the double stranded target DNA, through the first spacer of the first PEgRNA. In some embodiments, the first PEgRNA complexes with and directs a first prime editor to bind the double stranded target DNA at the position corresponding to the first search target sequence. In some embodiments, the second PEgRNA anneals with the second search target sequence on the second target strand of the double stranded target DNA, through a second spacer of the second PEgRNA. In some embodiments, the second PEgRNA complexes with and directs a second prime editor to bind the double stranded target DNA at the position corresponding to the second search target sequence. In some embodiments, the first prime editor and the second prime editor are the same. In some embodiments, the first prime editor and the second prime editor are different.

In some embodiments, the first search target sequence recognized by the spacer of the first PEgRNA and the second search target sequence recognized by the spacer of the second PEgRNA have a region of complementarity to each other. In some embodiments, the region of complementarity is 2 to 20 nucleotides in length. In some embodiments, the region of complementarity is 5 to 15 nucleotides in length.

In some embodiments, the first search target sequence recognized by the spacer of the first PEgRNA and the second search target sequence recognized by the spacer of the second PEgRNA do not have a region of complementarity to each other. In some embodiments, the positions of the first and second search target sequences relative to each other may be determined by their positions in the double stranded target DNA prior to editing. In some embodiments, the positions of the first and second search target sequences relative to each other may be determined by their positions in a reference double stranded target DNA.

In some embodiments, the 3′ end of the first search target sequence and the position corresponding to the 3′ end of the second search target sequence are 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleotides apart from each other. In some embodiments, the 3′ end of the first search target sequence and the position corresponding to the 3′ end of the second search target sequence are about 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 nucleotides apart from each other. In some embodiments, the 3′ end of the first search target sequence and the position corresponding to the 3′ end of the second search target sequence are about 500, 600, 700, 800, 900, 1000, 1100, 1200, 1300, 1400, 1500, 1600, 1700, 1800, 1900, 2000 or more nucleotides apart from each other. In some embodiments, the 3′ end of the first search target sequence and the position corresponding to the 3′ end of the second search target sequence are about 150 to about 450 nucleotides apart from each other. In some embodiments, the 3′ end of the first search target sequence and the position corresponding to the 3′ end of the second search target sequence are about 105 to about 145 nucleotides apart from each other. In some embodiments, the 3′ end of the first search target sequence and the position corresponding to the 3′ end of the second search target sequence are about 300 to about 3000 nucleotides apart from each other. In some embodiments, the 3′ end of the first search target sequence and the position corresponding to the 3′ end of the second search target sequence at least about 3000 nucleotides apart from each other.

In some embodiments, the bound first prime editor generates a first nick on the first PAM strand of the double stranded target DNA. In some embodiments, a first PEgRNA comprises a first primer binding site (PBS, also referred to herein as “primer binding site sequence”) that is complementary to the sequence of the first PAM strand of the double stranded target DNA that is immediately upstream of the first nick site, and can anneal with the sequence of the first PAM strand at a free 3′ end formed at the first nick site. In some embodiments, a first PEgRNA comprises a first primer binding site (PBS) that anneals to a free 3′ end formed at the first nick site and the first prime editor initiates DNA synthesis from the nick site, using the free 3′ end as a primer. In some embodiments, the first prime editor generates a first newly synthesized single stranded DNA encoded by a first editing template of the first PEgRNA.

In some embodiments, the bound second prime editor generates a second nick on the second PAM strand of the double stranded target DNA. In some embodiments, the double stranded target DNA, e.g., a target gene, comprises a double stranded DNA sequence between the first nick generated by the first prime editor on the second target strand (also referred to as the first PAM strand) and the second nick generated by the second prime editor on the first target strand (also referred to as the second PAM strand), which may be referred to as an inter-nick duplex (IND). In some embodiments, the two strands of an IND are perfectly complementary to each other. In some embodiments, the two strands of an IND are partially complementary to each other. In some embodiments, the IND is subsequently excised from the double stranded target DNA, e.g., the target gene.

In some embodiments, the IND is 1, 2, 3, 4, 5, 6, 7, 8, 9, 10, 11, 12, 13, 14, 15, 16, 17, 18, 19, 20 or more base pairs in length. In some embodiments, the IND is up to 5, up to 10, up to 15, up to 20, up to 25, up to 30, up to 40, or up to 50 base pairs in length. In some embodiments, the IND is 1-3000, 1-2500, 1-2000, 1-1500, 1-1000, 1-900, 1-800, 1-700, 1-600, 1-500, 1-400, 1-300, 1-200, 1-100, 1-50, 1-40, 1-30, 1-25, 1-20, 1-15, 1-10, or 1-5 base pairs in length. In some embodiments, the IND is 500-3000, 500-2500, 500-2000, 500-1500, 500-1000, 500-900, 500-800, 500-700, or 500-600 base pairs in length. In some embodiments, the IND is 30-300, 30-250, 30-200, 30-150, 30-100, 30-75, 30-50, 50-200, 50-150, 50-100, 50-75, 75-100, 75-150, 75-200, 75-250, 75-300 base pairs in length. In some embodiments, the IND is 1-3, 1-6, 1-9, 1-12, 1-15, 1-18, 1-21, 1-24, 1-27, 1-30, 1-36, 1-45, 1-60, 1-72, 1-90, 3-6, 3-9, 3-12, 3-15, 3-18, 3-21, 3-24, 3-27, 3-30, 3-36, 3-45, 3-60, 3-72, 3-90, 6-9, 6-12, 6-15, 6-18, 6-21, 6-24, 6-27, 6-30, 6-36, 6-45, 6-60, 6-72, 6-90, 9-12, 9-15, 9-18, 9-21, 9-24, 9-27, 9-30, 9-36, 9-45, 9-60, 9-72, 9-90, 12-15, 12-18, 12-21, 12-24, 12-27, 12-30, 12-36, 12-45, 12-60, 12-72, 12-90, 15-18, 15-21, 15-24, 15-27, 15-30, 15-36, 15-45, 15-60, 15-72, 15-90, 18-21, 18-24, 18-27, 18-30, 18-36, 18-45, 18-60, 18-72, 18-90, 21-24, 21-27, 21-30, 21-36, 21-45, 21-60, 21-72, 21-90, 24-27, 24-30, 24-36, 24-45, 24-60, 24-72, 24-90, 27-30, 27-36, 27-45, 27-60, 27-72, 27-90, 30-36, 30-45, 30-60, 30-72, 30-90, 45-60, 45-72, 60-72, 60-90, or 72-90 base pairs in length. In some embodiments, the IND is 1-3000, 1-2500, 1-2000, 1-1500, 1-1000, 1-900, 1-800, 1-700, 1-600, 1-500, 1-400, 1-300, 1-200, 1-100, 1-50, 1-40, 1-30, 1-25, 1-20, 1-15, 1-10, 1-5, 500-3000, 500-2500, 500-2000, 500-1500, 500-1000, 500-900, 500-800, 500-700, 500-600, 30-300, 30-250, 30-200, 30-150, 30-100, 30-75, 30-50, 50-200, 50-150, 50-100, 50-75, 75-100, 75-150, 75-200, 75-250, or 75-300 base pairs in length. In some embodiments, the IND is 1-3, 1-6, 1-9, 1-12, 1-15, 1-18, 1-21, 1-24, 1-27, 1-30, 1-36, 1-45, 1-60, 1-72, 1-90, 3-6, 3-9, 3-12, 3-15, 3-18, 3-21, 3-24, 3-27, 3-30, 3-36, 3-45, 3-60, 3-72, 3-90, 6-9, 6-12, 6-15, 6-18, 6-21, 6-24, 6-27, 6-30, 6-36, 6-45, 6-60, 6-72, 6-90, 9-12, 9-15, 9-18, 9-21, 9-24, 9-27, 9-30, 9-36, 9-45, 9-60, 9-72, 9-90, 12-15, 12-18, 12-21, 12-24, 12-27, 12-30, 12-36, 12-45, 12-60, 12-72, 12-90, 15-18, 15-21, 15-24, 15-27, 15-30, 15-36, 15-45, 15-60, 15-72, 15-90, 18-21, 18-24, 18-27, 18-30, 18-36, 18-45, 18-60, 18-72, 18-90, 21-24, 21-27, 21-30, 21-36, 21-45, 21-60, 21-72, 21-90, 24-27, 24-30, 24-36, 24-45, 24-60, 24-72, 24-90, 27-30, 27-36, 27-45, 27-60, 27-72, 27-90, 30-36, 30-45, 30-60, 30-72, 30-90, 45-60, 45-72, 60-72, 60-90, or 72-90 base pairs in length. In some embodiments, the IND is about 150 to about 450 base pairs in length. In some embodiments, the IND is about 105 to about 145 base pairs in length. In some embodiments, the IND is about 300 to about 3000 base pairs in length. In some embodiments, the IND is more than about 3000 base pairs in length.

In some embodiments, the double stranded target DNA is a double stranded target gene or a part of a double stranded target gene, and the IND comprises a part of a coding sequence of the target gene. In some embodiments, the IND comprises a part of a non-coding sequence of the target gene. In some embodiments, the IND comprises a part of an exon. In some embodiments, the IND comprises an entire exon. In some embodiments, the IND comprises a part of an intron. In some embodiments, the IND comprises an entire intron. In some embodiments, the IND comprises a 3′ UTR sequence of the target gene. In some embodiments, the IND comprises a 5′ UTR sequence of the target gene. In some embodiments, the IND comprises a whole or a part of an ORF of the target gene. In some embodiments, the IND comprises both coding and non-coding sequences of the target gene. In some embodiments, the IND comprises both intron and exon sequences of the target gene. For example, in some embodiments, the IND comprises the sequence of an exon flanked by an intronic sequence at the 5′ end, the 3′ end, or both ends. In some embodiments, the IND comprises one or more exons and intervening introns. In some embodiments, the IND comprises two or more exons and intervening introns. In some embodiments, the IND comprises all of the coding regions of a target gene, regulatory sequences of a target gene, or the entire target gene comprising its exons, introns and regulatory sequences. In some embodiments, the double stranded DNA comprises a gene or a part of a gene, and the IND comprises one or more mutations compared to a wild type reference sequence of the same gene. In some embodiments, the one or more mutations are associated with a disease. In some embodiments, the IND comprises an array of three nucleotide repeats (or tri-nucleotide repeats). In some embodiments, the IND comprises an array of tri-nucleotide repeats, wherein the number of the tri-nucleotide repeats is associated with a disease. As used herein, an array of tri-nucleotide repeats means at least two tri-nucleotides that are the same. In some embodiments, an array of tri-nucleotide repeats comprises at least 10, 20, 30, 50, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, or 1500 repeats of the same tri-nucleotides. In some embodiments, an array of tri-nucleotide repeats comprises at least 34 tri-nucleotide repeats. In some embodiments, an array of tri-nucleotide repeats comprises at least 50 tri-nucleotide repeats. In some embodiments, an array of tri-nucleotide repeats comprises at least 100 tri-nucleotide repeats. In some embodiments, an array of tri-nucleotide repeats comprises at least 1000 tri-nucleotide repeats. In some embodiments, an array of tri-nucleotide repeats is in a non-coding region of a gene, for example, a 3′ UTR, of a gene. In some embodiments, the array of tri-nucleotide repeats is an array of CTG (or the reverse complement CAG) repeats.

In some embodiments, a first PEgRNA comprises a first primer binding site (PBS) that is complementary to a free 3′ end of the second strand of the double stranded target DNA formed at the first nick site. In some embodiments, the first PBS anneals with the free 3′ end formed at the first nick site, and the first prime editor initiates DNA synthesis from the first nick site, using the free 3′ end at the first nick site as a primer. In some embodiments, the first prime editor synthesizes a first new single stranded DNA encoded by the first editing template of the first PEgRNA. In some embodiments, the second PEgRNA comprises a second PBS that is complementary to a free 3′ end of the first strand of the double stranded target DNA formed at the second nick site. In some embodiments, the second PBS anneals with the free 3′ end formed at the second nick site, and the second prime editor initiates DNA synthesis from the nick site, using the free 3′ end at the second nick site as a primer. In some embodiments, the second prime editor synthesizes a second newly synthesized single stranded DNA encoded by a second editing template of the second PEgRNA.

In some embodiments, through DNA repair, the sequence of the first newly synthesized single stranded DNA encoded by the first editing template and/or the sequence of the second newly synthesized single stranded DNA encoded by the second editing template is incorporated into the double stranded target DNA, e.g., a target gene, thereby incorporating one or more intended nucleotide edits in the double stranded target DNA, e.g., the target gene.

As used herein, a “nucleotide edit” or an “intended nucleotide edit” refers to a specified edit of a double stranded target DNA. A nucleotide edit or intended nucleotide edit refers to a (i) deletion of one or more contiguous nucleotides at one specific position, (ii) insertion of one or more contiguous nucleotides at one specific position, (iii) substitution of one or more contiguous nucleotides, or (iv) a combination of contiguous nucleotide substitutions, insertions and/or deletions of two or more contiguous nucleotides at one specific position, or other alterations at one specific position to be incorporated into the sequence of the double stranded target DNA. An intended nucleotide edit may refer to the edit on an editing template (e.g., a first editing template or a second editing template) as compared to the sequence of the double stranded target gene, or may refer to the edit encoded by an editing template in the newly synthesized single stranded DNA that is incorporated in the double stranded target DNA, e.g., the DMPK gene, as compared to endogenous sequence of the double stranded target DNA, e.g., the DMPK gene. In some embodiments, an intended nucleotide edit may also refer to the edit that results from incorporation of the newly synthesized DNA encoded by an editing template, or incorporation of the two newly synthesized single stranded DNA encoded by each of the first PEgRNA and the second PEgRNA in dual prime editing.

In some embodiments, the sequence of the first newly synthesized single stranded DNA and/or the sequence of the second newly synthesized single stranded DNA are incorporated into the double stranded target DNA, e.g., the target gene. In some embodiments, the first and/or the second newly synthesized single stranded DNAs comprises one or more intended nucleotide edits compared to the endogenous sequence of the double stranded target DNA, e.g., the target gene, which are incorporated in the double stranded target DNA, e.g., the target gene. In some embodiments, the sequence of the first newly synthesized single stranded DNA encoded by the first editing template is incorporated in the double stranded target DNA, e.g., the target gene, thereby incorporating one or more intended nucleotide edits in the double stranded target DNA, e.g., the target gene. In some embodiments, the sequence of the second newly synthesized single stranded DNA encoded by the second editing template is incorporated in the double stranded target DNA, e.g., the target gene, thereby incorporating one or more intended nucleotide edits in the double stranded target DNA, e.g., the target gene. In some embodiments, the sequence of the first newly synthesized single stranded DNA encoded by the first editing template and the sequence of the second newly synthesized single stranded DNA encoded by the second editing template are incorporated in the double stranded target DNA, e.g., the target gene, thereby incorporating one or more intended nucleotide edits in the double stranded target DNA, e.g., the target gene.

In some embodiments, the intended nucleotide edit comprises an insertion, deletion, nucleotide substitution, inversion, or any combination thereof compared to the endogenous sequence of the double stranded target DNA, e.g., the target gene. In some embodiments, the intended nucleotide edit comprises 1, 2, 3, 4, 5, 6, 7, 8, 9, 10, 11, 12, 13, 14, 15, 16, 17, 18, 19, 20 or more nucleotide substitutions compared to the endogenous sequence of the double stranded target DNA, e.g., the target gene. In some embodiments, the intended nucleotide edit comprises up to 5, up to 10, up to 15, up to 20, up to 25, up to 30, up to 40, or up to 50 nucleotide substitutions compared to the endogenous sequence of the double stranded target DNA, e.g., the target gene. In some embodiments, the intended nucleotide edit comprises 1-50, 1-40, 1-30, 1-25, 1-20, 1-15, 1-10, or 1-5 nucleotide substitutions compared to the endogenous sequence of the double stranded target DNA, e.g., the target gene. In some embodiments, the intended nucleotide edit comprises 3-50, 3-40, 3-30, 3-25, 3-20, 3-15, 3-10, or 3-5 nucleotide substitutions compared to the endogenous sequence of the double stranded target DNA, e.g., the target gene. In some embodiments, the intended nucleotide edit comprises 5-50, 5-40, 5-30, 5-25, 5-20, 5-15, or 5-10 nucleotide substitutions compared to the endogenous sequence of the double stranded target DNA, e.g., the target gene.

In some embodiments, the intended nucleotide edit comprises 1, 2, 3, 4, 5, 6, 7, 8, 9, 10, 11, 12, 13, 14, 15, 16, 17, 18, 19, 20 or more nucleotide insertions compared to the endogenous sequence of the double stranded target DNA, e.g., the target gene. In some embodiments, the intended nucleotide edit comprises up to 5, up to 10, up to 15, up to 20, up to 25, up to 30, up to 40, or up to 50 nucleotide insertions compared to the endogenous sequence of the double stranded target DNA, e.g., the target gene. In some embodiments, the intended nucleotide edit comprises single nucleotide insertions at 1, 2, 3, 4, 5, 6, 7, 8, 9, 10, 11, 12, 13, 14, 15, 16, 17, 18, 19, 20 or more sites in the double stranded target DNA compared to the endogenous sequence of the double stranded target DNA, e.g., the target gene. In some embodiments, the intended nucleotide edit comprises nucleotide insertions of greater than one nucleotide at each site in the double stranded target DNA compared to the endogenous sequence of the double stranded target DNA, e.g., the target gene. As used herein, “site” refers to a specific position in the sequence of a target DNA, e.g., a target gene. In some embodiments, the specific position in the sequence of the double stranded target DNA, e.g., a target gene, can be referred to by specific positions in a reference sequence, e.g., a wild type gene sequence. In some embodiments, a nucleotide insertion at position x refers to insertion of one or more nucleotides between position x and position x+1 as set forth by numbering in a reference sequence. In some embodiments, a nucleotide deletion at position x refers to deletion of the specific nucleotide at position x as set forth by numbering in a reference sequence. In some embodiments, a nucleotide deletion of positions x to x+n refers to deletion of the specific nucleotides starting at nucleotide x to nucleotide x+n, including nucleotide x and nucleotide x+n, as set forth by numbering in a reference sequence. In some embodiments, a nucleotide inversion of positions x to x+n refers to inversion of the specific nucleotides starting at nucleotide x to nucleotide x+n, including nucleotide x and nucleotide x+n, as set forth by numbering in a reference sequence.

In some embodiments, the intended nucleotide edit comprises nucleotide insertions of greater than one nucleotide at 1, 2, 3, 4, 5, 6, 7, 8, 9, 10, 11, 12, 13, 14, 15, 16, 17, 18, 19, 20 or more sites in the double stranded target DNA compared to the endogenous sequence of the double stranded target DNA, e.g., the target gene. In some embodiments, the intended nucleotide edit comprises 1-3000, 1-2500, 1-2000, 1-1500, 1-1000, 1-900, 1-800, 1-700, 1-600, 1-500, 1-400, 1-300, 1-200, 1-100, 1-50, 1-40, 1-30, 1-25, 1-20, 1-15, 1-10, or 1-5 nucleotide insertions compared to the endogenous sequence of the double stranded target DNA, e.g., the target gene. In some embodiments, the intended nucleotide edit comprises 500-3000, 500-2500, 500-2000, 500-1500, 500-1000, 500-900, 500-800, 500-700, or 500-600 nucleotide insertions compared to the endogenous sequence of the double stranded target DNA, e.g., the target gene. In some embodiments, the intended nucleotide edit comprises 30-300, 30-250, 30-200, 30-150, 30-100, 30-75, 30-50, 50-200, 50-150, 50-100, 50-75, 75-100, 75-150, 75-200, 75-250, or 75-300 nucleotide insertions compared to the endogenous sequence of the double stranded target DNA, e.g., the target gene. In some embodiments, the intended nucleotide edit comprises nucleotide insertions of 1-3000, 1-2500, 1-2000, 1-1500, 1-1000, 1-900, 1-800, 1-700, 1-600, 1-500, 1-400, 1-300, 1-200, 1-100, 1-50, 1-40, 1-30, 1-25, 1-20, 1-15, 1-10, 1-5, 500-3000, 500-2500, 500-2000, 500-1500, 500-1000, 500-900, 500-800, 500-700, 500-600, 30-300, 30-250, 30-200, 30-150, 30-100, 30-75, 30-50, 50-200, 50-150, 50-100, 50-75, 75-100, 75-150, 75-200, 75-250, or 75-300 nucleotides at each site in the double stranded target DNA compared to the endogenous sequence of the double stranded target DNA, e.g., the target gene.

In some embodiments, the intended nucleotide edit comprises 1, 2, 3, 4, 5, 6, 7, 8, 9, 10, 11, 12, 13, 14, 15, 16, 17, 18, 19, 20 or more nucleotide deletions compared to the endogenous sequence of the double stranded target DNA, e.g., the target gene. In some embodiments, the intended nucleotide edit comprises up to 5, up to 10, up to 15, up to 20, up to 25, up to 30, up to 40, or up to 50 nucleotide deletions compared to the endogenous sequence of the double stranded target DNA, e.g., the target gene. In some embodiments, the intended nucleotide edit comprises single nucleotide deletions at 1, 2, 3, 4, 5, 6, 7, 8, 9, 10, 11, 12, 13, 14, 15, 16, 17, 18, 19, 20 or more sites in the double stranded target DNA compared to the endogenous sequence of the double stranded target DNA, e.g., the target gene. In some embodiments, the intended nucleotide edit comprises nucleotide deletions of greater than one nucleotide at each site in the double stranded target DNA compared to the endogenous sequence of the double stranded target DNA, e.g., the target gene. In some embodiments, the intended nucleotide edit comprises nucleotide deletions of greater than one nucleotide at 1, 2, 3, 4, 5, 6, 7, 8, 9, 10, 11, 12, 13, 14, 15, 16, 17, 18, 19, 20 or more sites in the double stranded target DNA compared to the endogenous sequence of the double stranded target DNA, e.g., the target gene. In some embodiments, the intended nucleotide edit comprises 1-3000, 1-2500, 1-2000, 1-1500, 1-1000, 1-900, 1-800, 1-700, 1-600, 1-500, 1-400, 1-300, 1-200, 1-100, 1-50, 1-40, 1-30, 1-25, 1-20, 1-15, 1-10, or 1-5 nucleotide deletions compared to the endogenous sequence of the double stranded target DNA, e.g., the target gene. In some embodiments, the intended nucleotide edit comprises 500-3000, 500-2500, 500-2000, 500-1500, 500-1000, 500-900, 500-800, 500-700, or 500-600 nucleotide deletions compared to the endogenous sequence of the double stranded target DNA, e.g., the target gene. In some embodiments, the intended nucleotide edit comprises 30-300, 30-250, 30-200, 30-150, 30-100, 30-75, 30-50, 50-200, 50-150, 50-100, 50-75, 75-100, 75-150, 75-200, 75-250, 75-300 nucleotide deletions compared to the endogenous sequence of the double stranded target DNA, e.g., the target gene. In some embodiments, the intended nucleotide edit comprises deletion of about 105-145 nucleotides at a site of the target gene, e.g., the DMPK gene. In some embodiments, the intended nucleotide edit comprises deletion of about 150-450 nucleotides at a site of the target gene, e.g., the DMPK gene. In some embodiments, the intended nucleotide edit comprises deletion of about 300-3000 nucleotides at a site of the target gene, e.g., the DMPK gene. In some embodiments, the intended nucleotide edit comprises deletion of more than about 3000 nucleotides at a site of the target gene, e.g., the DMPK gene.

In some embodiments, the intended nucleotide edit comprises 1-3, 1-6, 1-9, 1-12, 1-15, 1-18, 1-21, 1-24, 1-27, 1-30, 1-36, 1-45, 1-60, 1-72, 1-90, 3-6, 3-9, 3-12, 3-15, 3-18, 3-21, 3-24, 3-27, 3-30, 3-36, 3-45, 3-60, 3-72, 3-90, 6-9, 6-12, 6-15, 6-18, 6-21, 6-24, 6-27, 6-30, 6-36, 6-45, 6-60, 6-72, 6-90, 9-12, 9-15, 9-18, 9-21, 9-24, 9-27, 9-30, 9-36, 9-45, 9-60, 9-72, 9-90, 12-15, 12-18, 12-21, 12-24, 12-27, 12-30, 12-36, 12-45, 12-60, 12-72, 12-90, 15-18, 15-21, 15-24, 15-27, 15-30, 15-36, 15-45, 15-60, 15-72, 15-90, 18-21, 18-24, 18-27, 18-30, 18-36, 18-45, 18-60, 18-72, 18-90, 21-24, 21-27, 21-30, 21-36, 21-45, 21-60, 21-72, 21-90, 24-27, 24-30, 24-36, 24-45, 24-60, 24-72, 24-90, 27-30, 27-36, 27-45, 27-60, 27-72, 27-90, 30-36, 30-45, 30-60, 30-72, 30-90, 45-60, 45-72, 60-72, 60-90, or 72-90 nucleotide deletions compared to the endogenous sequence of the double stranded target DNA, e.g., the target gene. In some embodiments, the intended nucleotide edit comprises nucleotide deletions of 1-3000, 1-2500, 1-2000, 1-1500, 1-1000, 1-900, 1-800, 1-700, 1-600, 1-500, 1-400, 1-300, 1-200, 1-100, 1-50, 1-40, 1-30, 1-25, 1-20, 1-15, 1-10, 1-5, 500-3000, 500-2500, 500-2000, 500-1500, 500-1000, 500-900, 500-800, 500-700, 500-600, 30-300, 30-250, 30-200, 30-150, 30-100, 30-75, 30-50, 50-200, 50-150, 50-100, 50-75, 75-100, 75-150, 75-200, 75-250, or 75-300 nucleotides at each site in the double stranded target DNA compared to the endogenous sequence of the double stranded target DNA, e.g., the target gene. In some embodiments, the intended nucleotide edit comprises nucleotide deletions of 1-3, 1-6, 1-9, 1-12, 1-15, 1-18, 1-21, 1-24, 1-27, 1-30, 1-36, 1-45, 1-60, 1-72, 1-90, 3-6, 3-9, 3-12, 3-15, 3-18, 3-21, 3-24, 3-27, 3-30, 3-36, 3-45, 3-60, 3-72, 3-90, 6-9, 6-12, 6-15, 6-18, 6-21, 6-24, 6-27, 6-30, 6-36, 6-45, 6-60, 6-72, 6-90, 9-12, 9-15, 9-18, 9-21, 9-24, 9-27, 9-30, 9-36, 9-45, 9-60, 9-72, 9-90, 12-15, 12-18, 12-21, 12-24, 12-27, 12-30, 12-36, 12-45, 12-60, 12-72, 12-90, 15-18, 15-21, 15-24, 15-27, 15-30, 15-36, 15-45, 15-60, 15-72, 15-90, 18-21, 18-24, 18-27, 18-30, 18-36, 18-45, 18-60, 18-72, 18-90, 21-24, 21-27, 21-30, 21-36, 21-45, 21-60, 21-72, 21-90, 24-27, 24-30, 24-36, 24-45, 24-60, 24-72, 24-90, 27-30, 27-36, 27-45, 27-60, 27-72, 27-90, 30-36, 30-45, 30-60, 30-72, 30-90, 45-60, 45-72, 60-72, 60-90, or 72-90 nucleotides at each site in the double stranded target DNA compared to the endogenous sequence of the double stranded target DNA, e.g., the target gene.

In some embodiments, the intended nucleotide edits, e.g., nucleotide substitutions, insertions, or deletions, are in consecutive or contiguous nucleotides in the double stranded target DNA sequence compared to the endogenous sequence of the double stranded target DNA, e.g., the target gene. In some embodiments, the intended nucleotide edits, e.g., nucleotide substitutions, insertions, or deletions are in non-consecutive or non-contiguous nucleotides in the double stranded target DNA sequence compared to the endogenous sequence of the double stranded target DNA, e.g., the target gene.

In some embodiments, the intended nucleotide edit comprises an inversion as compared to the endogenous sequence of the double stranded target DNA, e.g., the target gene. In some embodiments, a segment of 2, 3, 4, 5, 6, 7, 8, 9, 10, 20, 30, 40, 50, 60, 70, 80, 90, 100, 110, 120, 130, 140, 150, 200, 250, 300 or more nucleotides of the endogenous sequence of the double stranded target DNA is inverted. In some embodiments, a segment of 1-50, 1-40, 1-30, 1-25, 1-20, 1-15, 1-10, or 1-5 nucleotides of the endogenous sequence of the double stranded target DNA is inverted. In some embodiments, a segment of 3-50, 3-40, 3-30, 3-25, 3-20, 3-15, 3-10, 3-5, 5-50, 5-40, 5-30, 5-25, 5-20, 5-15, or 5-10 nucleotides of the endogenous sequence of the double stranded target DNA is inverted.

In some embodiments, the intended nucleotide edit comprises more than one nucleotide edit in the double stranded target DNA sequence compared to the endogenous sequence of the double stranded target DNA, e.g., the target gene. In some embodiments, the intended nucleotide edit comprises a combination of one or more of nucleotide substitutions, one or more of nucleotide insertions, one or more of nucleotide deletions and one or more of nucleotide inversions compared to the endogenous sequence of the double stranded target DNA, e.g., the target gene. In some embodiments, the intended nucleotide edit comprises one or more nucleotide substitutions and one or more nucleotide insertions. In some embodiments, the intended nucleotide edit comprises one or more nucleotide substitutions and one or more nucleotide deletions. In some embodiments, the intended nucleotide edit comprises one or more nucleotide substitutions and one or more nucleotide inversions. In some embodiments, the intended nucleotide edit comprises one or more nucleotide insertions and one or more nucleotide deletions. In some embodiments, the intended nucleotide edit comprises one or more nucleotide insertions and one or more nucleotide inversions. In some embodiments, the intended nucleotide edit comprises one or more nucleotide deletions and one or more nucleotide inversions. In some embodiments, the intended nucleotide edit comprises one or more nucleotide substitutions, one or more nucleotide insertions and one or more nucleotide deletions. In some embodiments, the intended nucleotide edit comprises one or more nucleotide substitutions, one or more nucleotide insertions and one or more nucleotide inversions. In some embodiments, the intended nucleotide edit comprises one or more nucleotide substitutions, one or more nucleotide deletions and one or more nucleotide inversions. In some embodiments, the intended nucleotide edit comprises one or more nucleotide insertions, one or more nucleotide deletions and one or more nucleotide inversions. In some embodiments, the intended nucleotide edit comprises one or more nucleotide substitutions, one or more nucleotide insertions, one or more nucleotide deletions and one or more nucleotide inversions.

In some embodiments, the first newly synthesized single stranded DNA and the second newly synthesized single stranded DNA have a region of complementarity to each other. In some embodiments, the first newly synthesized single stranded DNA has a region of complementarity to an endogenous sequence of the double stranded target DNA, e.g., the target gene, adjacent to or near a nick site. In some embodiments, the first newly synthesized single stranded DNA has a region of complementarity to an endogenous sequence of the double stranded target DNA, e.g., the target gene, on the first strand adjacent to the second nick site. In some embodiments, the first newly synthesized single stranded DNA has a region of complementarity to an endogenous sequence of the double stranded target DNA, e.g., the target gene, on the first strand adjacent to and downstream of the second nick site. In some embodiments, the first newly synthesized single stranded DNA has a region of identity to an endogenous sequence of the double stranded target DNA e.g., the target gene, on the second strand adjacent to and downstream of the first nick site. In some embodiments, the first newly synthesized single stranded DNA has a region of identity to an endogenous sequence of the double stranded target on the second strand adjacent to and upstream of the second nick site.

In some embodiments, the second newly synthesized single stranded DNA has a region of complementarity to an endogenous sequence of the double stranded target DNA, e.g., the target gene, adjacent to or near a nick site. In some embodiments, the second newly synthesized single stranded DNA has a region of complementarity to an endogenous sequence of the double stranded target DNA, e.g., the target gene, on the second strand adjacent to the first nick site. In some embodiments, the second newly synthesized single stranded DNA has a region of complementarity to an endogenous sequence of the double stranded target DNA, e.g., the target gene, on the second strand adjacent to and downstream of the first nick site. In some embodiments, the second newly synthesized single stranded DNA has a region of identity to an endogenous sequence of the double stranded target DNA, e.g., the target gene, on the first strand adjacent to and upstream of the second nick site.

As used herein, reference for positioning in a chromosome or a double stranded polynucleotide, e.g., a double stranded target DNA, includes the position on either strand of the two strands, unless otherwise specified. For example, a position of a first nick site may be used refer to the first nick site on the first edit strand and/or the corresponding position on the second edit strand.

By “upstream” and “downstream” it is intended to define relative positions of at least two nucleotides, regions, or sequences in a nucleic acid molecule oriented in a 5′-to-3′ direction according to a reference strand of the nucleic acid molecule. For example, a first nucleotide is upstreat of a second nucleotide when the first nucleotide is 5′ to the second nucleotide. A first sequence is upstream of a second sequence in a DNA molecule where the first sequence is positioned 5′ to the second sequence according to a reference strand of the DNA molecule. Accordingly, the second sequence is downstream, that is, 3′, of the first sequence.

In some embodiments, each of the first newly synthesized single stranded DNA and the second newly synthesized single stranded DNA has a region of complementarity to an endogenous sequence of the double stranded target DNA, e.g., the target gene, adjacent to or near a nick site. In some embodiments, each of the first newly synthesized single stranded DNA and the second newly synthesized single stranded DNA has a region of identity to an endogenous sequence of the double stranded target DNA, e.g., the target gene, adjacent to or near a nick site. In some embodiments, the first newly synthesized single stranded DNA has a region of complementarity to an endogenous sequence of the double stranded target DNA, e.g., the target gene, on the first strand adjacent to and upstream of the second nick site, and the second newly synthesized single stranded DNA has a region of complementarity to an endogenous sequence of the double stranded target DNA, e.g., the target gene, on the second strand adjacent to and upstream of the first nick site. In some embodiments, the first newly synthesized single stranded DNA has a region of identity to an endogenous sequence of the double stranded target DNA on the second strand adjacent to and downstream of the first nick site and/or a region of identity to an endogenous sequence of the double stranded target DNA on the second strand adjacent to and downstream of the position corresponding to the second nick site, and the second newly synthesized single stranded DNA has a region of identity to an endogenous sequence of the double stranded target DNA on the first strand adjacent to and downstream of the first nick site and/or a region of identity to an endogenous sequence of the double stranded target DNA on the first strand adjacent to and downstream of the position corresponding to the first nick site.

In some embodiments, the first newly synthesized single stranded DNA encoded by the first editing template and the second newly synthesized single stranded DNA encoded by the second editing template have a region of complementarity to each other. The complementary region between the first newly synthesized single stranded DNA and the second newly synthesized single stranded DNA may be referred to as an overlap duplex (OD).

In some embodiments, the first newly synthesized single stranded DNA encoded by the first editing template and the second newly synthesized single stranded DNA encoded by the second editing template are substantially complementary to each other. In some embodiments, the OD is incorporated in the double stranded target DNA, e.g., the target gene, thereby incorporating one or more intended nucleotide edits encoded by the first editing template and the second editing template into the double stranded target DNA, e.g., the target gene. In some embodiments, the OD replaces all or a portion of the IND, thereby incorporating one or more intended nucleotide edits in the double stranded target DNA, e.g., the target gene. In some embodiments, the IND is excised or degraded, and the OD is incorporated at the place of the IND excision, followed by ligation of the nicks on both strands of the double stranded target DNA, e.g., the target gene, thereby incorporating the one or more intended nucleotide edits in the double stranded target DNA. In some embodiments, the sequence of the OD comprises partial identity compared to the sequence of the IND. In some embodiments, the sequence of the OD comprises no identity compared to the sequence of the IND. In some embodiments, the sequence of the OD comprises a sequence exogenous to the double stranded target DNA.

In some embodiments, the first editing template and the second editing template comprise a region of complementarity or substantial complementarity to each other, and do not have complementarity to either strand of the double stranded target DNA, e.g., the target gene. Accordingly, in some embodiments, the first newly synthesized single stranded DNA encoded by the first editing template and the second newly synthesized single stranded DNA encoded by the second editing template can anneal to each other to form an OD that does not have substantial sequence identity with the endogenous sequence of double stranded target DNA, e.g., the target gene. In some embodiments, the sequence of the OD comprises a sequence exogenous to the double stranded target DNA, e.g., the target gene. In some embodiments, the sequence of the OD consists of a sequence exogenous to the double stranded target DNA, e.g., the target gene. In some embodiments, the IND is excised, and the OD is incorporated at the place of the IND excision, followed by ligation of the nicks on both strands of the target DNA, thereby incorporating the sequence of the OD in the double stranded target DNA.

In some embodiments, the OD comprises 1, 2, 3, 4, 5, 6, 7, 8, 9, 10, 11, 12, 13, 14, 15, 16, 17, 18, 19, 20, 21, 22, 23, 24, 25, 30, 35, 40, 45, 50 or more contiguous complementary or substantially complementary base pairs. In some embodiments, the OD comprises about 5 to 10, 5 to 15, 5 to 20, 5 to 25, 5 to 30, 5 to 35, 5 to 40, 5 to 45, 5 to 50, 5 to 55, 5 to 60, 5 to 65, 5 to 70, 5 to 75, 5 to 80, 5 to 85, 5 to 90, 5 to 95, 5 to 100, 5 to 110, 5 to 120, 5 to 130, 5 to 140, 5 to 150, 15 to 20, 15 to 25, 15 to 30, 15 to 35, 15 to 40, 15 to 45, 15 to 50, 15 to 55, 15 to 60, 15 to 65, 15 to 70, 15 to 75, 15 to 80, 15 to 85, 15 to 90, 15 to 95, 15 to 100, 15 to 110, 15 to 120, 15 to 130, 15 to 140, 15 to 150, 25 to 30, 25 to 35, 25 to 40, 25 to 45, 25 to 50, 25 to 55, 25 to 60, 25 to 65, 25 to 70, 25 to 75, 25 to 80, 25 to 85, 25 to 90, 25 to 95, 25 to 100, 25 to 110, 25 to 120, 25 to 130, 25 to 140, 25 to 150, 35 to 40, 35 to 45, 35 to 50, 35 to 55, 35 to 60, 35 to 65, 35 to 70, 35 to 75, 35 to 80, 35 to 85, 35 to 90, 35 to 95, 35 to 100, 35 to 110, 35 to 120, 35 to 130, 35 to 140, 35 to 150, 45 to 50, 45 to 55, 45 to 60, 45 to 65, 45 to 70, 45 to 75, 45 to 80, 45 to 85, 45 to 90, 45 to 95, 45 to 100, 45 to 110, 45 to 120, 45 to 130, 45 to 140, o45 to 150, 55 to 60, 55 to 65, 55 to 70, 55 to 75, 55 to 80, 55 to 85, 55 to 90, 55 to 95, 55 to 100, 55 to 110, 55 to 120, 55 to 130, 55 to 140, 55 to 150, 65 to 70, 65 to 75, 65 to 80, 65 to 85, 65 to 90, 65 to 95, 65 to 100, 65 to 110, 65 to 120, 65 to 130, 65 to 140, 65 to 150, 75 to 80, 75 to 85, 75 to 90, 75 to 95, 75 to 100, 75 to 110, 75 to 120, 75 to 130, 75 to 140, 75 to 150, 85 to 90, 85 to 95, 85 to 100, 85 to 110, 85 to 120, 85 to 130, 85 to 140, 85 to 150, 95 to 100, 95 to 110, 95 to 120, 95 to 130, 95 to 140, 95 to 150, 105 to 110, 105 to 120, 105 to 130, 105 to 140, 105 to 150, 115 to 120, 115 to 130, 115 to 140, 115 to 150, 125 to 130, 125 to 140, 125 to 150, 135 to 140, 135 to 150, or 145 to 150 contiguous complementary or substantially complementary base pairs. In some embodiments, the OD comprise 10, 11, 12, 13, 14, 15, 16, 17, 18, 19, 20, 21, 22, 23, 24 or 25 contiguous complementary or substantially complementary base pairs. In some embodiments, the OD comprise 30, 35, 40, 50, 60, 70, 80, 90, or 100 contiguous complementary or substantially complementary base pairs. In some embodiments, the OD comprise no greater than 20, 25, 30, 35, 40, 50, 60, 70, 80, 90, or 100 contiguous complementary or substantially complementary base pairs. In some embodiments, the OD comprises a sufficient number of contiguous complementary base pairs to form a sufficiently stable duplex for replacement of the IND. In some embodiments, the OD comprises at least 10 contiguous complementary or substantially complementary base pairs. In some embodiments, the OD comprises at least 15 contiguous complementary or substantially complementary base pairs. In some embodiments, the OD comprises about 20 contiguous complementary or substantially complementary base pairs.

In some embodiments, the OD contains about 20 to 40 contiguous complementary or substantially complementary base pairs. In some embodiments, the OD contains about 20, about 30, about 40, about 50, about 60, about 70, or about 80 contiguous complementary or substantially complementary base pairs. In some embodiments, the OD contains about 10 to 19 contiguous complementary or substantially complementary base pairs. In some embodiments, the OD contains about 20 to 30 contiguous complementary or substantially complementary base pairs. In some embodiments, the OD contains about 30 to 40 contiguous complementary or substantially complementary base pairs. In some embodiments, the OD contains at least 40 contiguous complementary or substantially complementary base pairs. In some embodiments, the OD consists of about 20 to 40 contiguous complementary or substantially complementary base pairs. In some embodiments, the OD consists of about 20, about 30, about 40, about 50, about 60, about 70, or about 80 contiguous complementary or substantially complementary base pairs. In some embodiments, the OD consists of about 10 to 19 contiguous complementary or substantially complementary base pairs. In some embodiments, the OD consists of about 20 to 30 contiguous complementary or substantially complementary base pairs. In some embodiments, the OD consists of about 30 to 40 contiguous complementary or substantially complementary base pairs. In some embodiments, the OD consists of 23, 38, 53, 68, or 83 contiguous complementary or substantially complementary base pairs. In some embodiments, the OD consists of 38 contiguous complementary or substantially complementary base pairs.

The sequence of the OD can comprises any exogenous sequence or any endogenous sequence of the DMPK gene. The GC content of the OD may vary. In some embodiments, the GC content of the OD is less than about 45%. In some embodiments, the GC content of the OD is about 45%-60%. In some embodiments, the GC content of the OD is about 60%-75%. In some embodiments, the GC content of the OD is at least about 75%. In some embodiments, the GC content of the OD is about 40%-80%. In some embodiments, the GC content of the OD is about 50%-60%. In some embodiments, the GC content of the OD is about 60%-80%. In some embodiments, the GC content of the OD is about 60%-70%. In some embodiments, the GC content of the OD is about 70%-80%. In some embodiments, the GC content of the OD is about 10%-20%, about 20%-30%, about 30%-40%, about 40%-50%, about 50%-60%, about 60%-70%, about 70%-80%, about 80%-90% or about 90%-100%. In some embodiments, the GC content of the OD is about 42%. In some embodiments, the GC content of the OD is about 53%. In some embodiments, the GC content of the OD is about 63%. In some embodiments, the GC content of the OD is about 71%. In some embodiments, the GC content of the OD is about 79%. In some embodiments, the GC content of the OD is about 63%.

In some embodiments, the OD replaces the IND of a target DNA, wherein the double stranded target DNA is an entire target gene or is part of a target gene. In some embodiments, the OD replaces part of an exon or an entire exon, part of an intron or an entire intron, one or more exons and intervening introns, all of the coding regions of a target gene, regulatory sequences of a target gene, or the entire target gene comprising its exons, introns and regulatory sequences. In some embodiments, the OD comprises a region of identity to an endogenous sequence of the double stranded target DNA. In some embodiments, the OD does not have sequence identity to an endogenous sequence of the double stranded target DNA. In some embodiments, the OD is exogenous to the double stranded target DNA, e.g., the target gene.

In some embodiments, the OD has a biological function or encodes a polypeptide having a biological function, or a portion thereof. In some embodiments, the OD comprises an expression cassette. In some embodiments, the OD comprises a nucleotide sequence that encodes an expression tag, for example, an affinity tag, a His tag, a V5 tag, or a FLAG tag. In some embodiments, the OD comprises a nucleotide sequence that encodes a His tag. In some embodiments, the OD comprises a nucleotide sequence that encodes a FLAG tag. In some embodiments, the OD comprises a nucleotide sequence that encodes an attB or an attP sequence. In some embodiments, the OD comprises a nucleotide sequence that encodes a reporter protein, for example, a green fluorescence protein, a blue fluorescence protein, a cyan fluorescence protein, a yellow fluorescence protein, an auto fluorescent protein, or a luciferase. In some embodiments, the OD comprises a recognition site of an enzyme, for example, a recombinase recognition sequence. In some embodiments, the OD comprises nucleotide sequence that encodes a selectable marker, for example, an antibiotic resistance marker. In some embodiments, the OD comprises a regulatory sequence, for example, a promoter, an enhancer, or an insulator. In some embodiments, the OD comprises a trackable sequence, for example, a barcode. In some embodiments, replacement of the IND by the OD restores or partially restores the function of the target gene. In some embodiments, the target gene is a disease-associated gene. In some embodiments, the target gene is a monogenic disease-associated gene. In some embodiments, the target gene is a polygenic disease-associated gene. In some embodiments, the target gene is a disease-associated gene containing one or more disease-causing mutations, wherein replacement of the IND by the OD corrects the mutations, thereby restoring or partially restoring the function of the target gene. In some embodiments, the disease-associated gene containing one or more disease-causing mutations is in a human subject in need of treatment. In some embodiments, the target gene is a mutated gene causing a disease or disorder in a human subject, wherein replacement of the IND by the OD corrects the mutated gene, thereby restoring or partially restoring the function of the target gene. In some embodiments, the target gene is a disease-associated gene containing one or more disease-causing mutations, wherein replacement of the IND by the OD modifies the target gene to restore or partially restore the function of the target gene. In some embodiments, the disease-associated gene containing one or more disease-causing mutations is in a human subject in need of treatment. In some embodiments, the target gene is a mutated gene causing a disease or disorder in a human subject, wherein replacement of the IND by the OD modifies the mutated gene to restore or partially restore the function of the target gene.

In some embodiments, the first editing template and/or the second editing template comprises a nucleotide sequence that encodes a polypeptide sequence that is the same as, or a portion of, the polypeptide sequence encoded by the IND, but does not have substantial nucleotide sequence complementarity or identity to the sequence of the IND. In some embodiments, the second newly synthesized single stranded DNA encoded by the second editing template comprises a nucleotide sequence that encodes a polypeptide sequence that is the same as, or a portion of, the polypeptide sequence encoded by the IND, but does not have substantial nucleotide sequence complementarity or identity to the sequence of the IND. Accordingly, in some embodiments, the OD comprises a sequence that encodes a polypeptide sequence that is the same as the polypeptide sequence or a portion of the same polypeptide sequence encoded by the IND, wherein the OD does not have substantial nucleotide sequence identity to the sequence of the IND.

In some embodiments, the first newly synthesized single stranded DNA encoded by the first editing template and the second newly synthesized single stranded DNA encoded by the second editing template comprises a region of complementarity with each other, and can anneal with each other to form an OD. In some embodiments, the first newly synthesized single stranded DNA encoded by the first editing template further comprises a region that does not have complementarity with the second newly synthesized single stranded DNA encoded by the second editing template (see exemplary schematic in FIG. 4B). In some embodiments, the second newly synthesized single stranded DNA encoded by the second editing template further comprises a region that does not have complementarity with the first newly synthesized single stranded DNA encoded by the first editing template. Accordingly, in some embodiments, the first newly synthesized single stranded DNA encoded by the first editing template and the second newly synthesized single stranded DNA encoded by the second editing template can anneal to each other through the partially complementary sequences to form an OD that is linked to a 5′ overhang and/or a 3′ overhang. In some embodiments, the IND is removed, the OD, along with the 5′ overhang and/or the 3′ overhang, is incorporated at the place of the IND excision in the double stranded target DNA, e.g., the target gene. Through DNA repair, the gaps corresponding to the positions of the 5′ overhang and/or the 3′ overhangs are filled and ligated, thereby incorporating the one or more intended nucleotide edits in the double stranded target DNA, e.g., the target gene.

Accordingly, in some embodiments, the IND is replaced by the sequence of (A+C), (B+C), or (A+B+C), wherein A is the region, and its complementary strand, of the first newly synthesized single stranded DNA that is not complementary to the second newly synthesized single stranded DNA, wherein B is the region, and its complementary strand, of the second newly synthesized single stranded DNA that is not complementary to the first newly synthesized single stranded DNA, and wherein C is the OD. The double stranded sequence of (A+C), (B+C), or (A+B+C) that replaces the IND may be referred to as the “replacement duplex (RD)”.

Accordingly, in some embodiments, the RD comprises the OD. In some embodiments, as exemplified in FIG. 4A, the first editing template and the second editing template are substantially complementary to each other. Accordingly, in some embodiments, the OD comprises the entirety or substantially the entirety of the first newly synthesized single stranded DNA encoded by the first editing template and the second newly synthesized single stranded DNA encoded by the second editing template. In some embodiments, the RD consists of the OD. In some embodiments, as exemplified in FIG. 4B, the RD comprises the OD, the non-complementary region of the first newly synthesized DNA compared to the second newly synthesized DNA and complement thereof, and/or the non-complementary region of the second newly synthesized DNA compared to the first newly synthesized DNA and complement thereof.

In some embodiments, the RD comprises 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 60, 70, 80, 90, 100, 110, 120, 130, 140, 150, 160, 170, 180, 190, 200 or more base pairs. In some embodiments, the RD comprises about 5 to 10, 5 to 15, 5 to 20, 5 to 25, 5 to 30, 5 to 35, 5 to 40, 5 to 45, 5 to 50, 5 to 55, 5 to 60, 5 to 65, 5 to 70, 5 to 75, 5 to 80, 5 to 85, 5 to 90, 5 to 95, 5 to 100, 5 to 110, 5 to 120, 5 to 130, 5 to 140, 5 to 150, 5 to 175, 5 to 200, 5 to 225, 5 to 250, 5 to 275, 5 to 300, 5 to 325, 5 to 350, 5 to 375, 5 to 400, 5 to 425, 5 to 450, 5 to 475, 5 to 500, 10 to 15, 10 to 20, 10 to 25, 10 to 30, 10 to 35, 10 to 40, 10 to 45, 10 to 50, 10 to 55, 10 to 60, 10 to 65, 10 to 70, 10 to 75, 10 to 80, 10 to 85, 10 to 90, 10 to 95, 10 to 100, 10 to 110, 10 to 120, 10 to 130, 10 to 140, 10 to 150, 10 to 175, 10 to 200, 10 to 225, 10 to 250, 10 to 275, 10 to 300, 10 to 325, 10 to 350, 10 to 375, 10 to 400, 10 to 425, 10 to 450, 10 to 475, 10 to 500, 15 to 20, 15 to 25, 15 to 30, 15 to 35, 15 to 40, 15 to 45, 15 to 50, 15 to 55, 15 to 60, 15 to 65, 15 to 70, 15 to 75, 15 to 80, 15 to 85, 15 to 90, 15 to 95, 15 to 100, 15 to 110, 15 to 120, 15 to 130, 15 to 140, 15 to 150, 15 to 175, 15 to 200, 15 to 225, 15 to 250, 15 to 275, 15 to 300, 15 to 325, 15 to 350, 15 to 375, 15 to 400, 15 to 425, 15 to 450, 15 to 475, 15 to 500, 20 to 25, 20 to 30, 20 to 35, 20 to 40, 20 to 45, 20 to 50, 20 to 55, 20 to 60, 20 to 65, 20 to 70, 20 to 75, 20 to 80, 20 to 85, 20 to 90, 20 to 95, 20 to 100, 20 to 110, 20 to 120, 20 to 130, 20 to 140, 20 to 150, 20 to 175, 20 to 200, 20 to 225, 20 to 250, 20 to 275, 20 to 300, 20 to 325, 20 to 350, 20 to 375, 20 to 400, 20 to 425, 20 to 450, 20 to 475, 20 to 500, 30 to 35, 30 to 40, 30 to 45, 30 to 50, 30 to 55, 30 to 60, 30 to 65, 30 to 70, 30 to 75, 30 to 80, 30 to 85, 30 to 90, 30 to 95, 30 to 100, 30 to 110, 30 to 120, 30 to 130, 30 to 140, 30 to 150, 30 to 175, 30 to 200, 30 to 225, 30 to 250, 30 to 275, 30 to 300, 30 to 325, 30 to 350, 30 to 375, 30 to 400, 30 to 425, 30 to 450, 30 to 475, 30 to 500, 40 to 45, 40 to 50, 40 to 55, 40 to 60, 40 to 65, 40 to 70, 40 to 75, 40 to 80, 40 to 85, 40 to 90, 40 to 95, 40 to 100, 40 to 110, 40 to 120, 40 to 130, 40 to 140, 40 to 150, 40 to 175, 40 to 200, 40 to 225, 40 to 250, 40 to 275, 40 to 300, 40 to 325, 40 to 350, 40 to 375, 40 to 400, 40 to 425, 40 to 450, 40 to 475, 40 to 500, 50 to 55, 50 to 60, 50 to 65, 50 to 70, 50 to 75, 50 to 80, 50 to 85, 50 to 90, 50 to 95, 50 to 100, 50 to 110, 50 to 120, 50 to 130, 50 to 140, 50 to 150, 50 to 175, 50 to 200, 50 to 225, 50 to 250, 50 to 275, 50 to 300, 50 to 325, 50 to 350, 50 to 375, 50 to 400, 50 to 425, 50 to 450, 50 to 475, 50 to 500, 75 to 80, 75 to 85, 75 to 90, 75 to 95, 75 to 100, 75 to 110, 75 to 120, 75 to 130, 75 to 140, 75 to 150, 75 to 175, 75 to 200, 75 to 225, 75 to 250, 75 to 275, 75 to 300, 75 to 325, 75 to 350, 75 to 375, 75 to 400, 75 to 425, 75 to 450, 75 to 475, 75 to 500, 100 to 110, 100 to 120, 100 to 130, 100 to 140, 100 to 150, 100 to 175, 100 to 200, 100 to 225, 100 to 250, 100 to 275, 100 to 300, 100 to 325, 100 to 350, 100 to 375, 100 to 400, 100 to 425, 100 to 450, 100 to 475, 100 to 500, 125 to 150, 125 to 175, 125 to 200, 125 to 225, 125 to 250, 125 to 275, 125 to 300, 125 to 325, 125 to 350, 125 to 375, 125 to 400, 125 to 425, 125 to 450, 125 to 475, 125 to 500, 150 to 175, 150 to 200, 150 to 225, 150 to 250, 150 to 275, 150 to 300, 150 to 325, 150 to 350, 150 to 375, 150 to 400, 150 to 425, 150 to 450, 150 to 475, 150 to 500, 175 to 200, 175 to 225, 175 to 250, 175 to 275, 175 to 300, 175 to 325, 175 to 350, 175 to 375, 175 to 400, 175 to 425, 175 to 450, 175 to 475, 175 to 500, 200 to 250, 200 to 275, 200 to 300, 200 to 325, 200 to 350, 200 to 375, 200 to 400, 200 to 425, 200 to 450, 200 to 475, 200 to 500, 225 to 250, 225 to 275, 225 to 300, 225 to 325, 225 to 350, 225 to 375, 225 to 400, 225 to 425, 225 to 450, 225 to 475, 225 to 500, 250 to 275, 250 to 300, 275 to 300, 275 to 325, 275 to 350, 275 to 375, 275 to 400, 275 to 425, 275 to 450, 275 to 475, 275 to 500, 300 to 325, 300 to 350, 300 to 375, 300 to 400, 300 to 425, 300 to 450, 300 to 475, 300 to 500, 325 to 350, 325 to 375, 325 to 400, 325 to 425, 325 to 450, 325 to 475, 325 to 500, 350 to 375, 350 to 400, 350 to 425, 350 to 450, 350 to 475, 350 to 500, 375 to 400, 375 to 425, 375 to 450, 375 to 475, 375 to 500, 400 to 425, 400 to 450, 400 to 475, 400 to 500, 425 to 450, 425 to 475, 425 to 500, 450 to 475, 450 to 500, or 475 to 500 base pairs. In some embodiments, the RD comprise 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 base pairs. In some embodiments, the RD comprise at least 30, 35, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 base pairs. In some embodiments, the RD comprise no greater than 20, 25, 30, 35, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 base pairs.

In some embodiments, the RD replaces the IND of a target DNA, wherein the IND is an entire target gene or is part of a target gene. In some embodiments, the RD replaces part of an exon or an entire exon, part of an intron or an entire intron, one or more exons and intervening introns, all of the coding regions of a target gene, regulatory sequences of a target gene, or the entire target gene comprising its exons, introns and regulatory sequences, thereby incorporating the one or more intended nucleotide edits compared to the endogenous sequence of the double stranded target DNA, e.g., the DMPK gene. In some embodiments, the RD comprises a region of identity to an endogenous sequence of the double stranded target DNA. In some embodiments, the RD does not have sequence identity to an endogenous sequence of the double stranded target DNA. In some embodiments, the RD is exogenous to the double stranded target DNA, e.g., the target gene. Accordingly, in some embodiments, the intended nucleotide edit(s) comprises replacement of an endogenous sequence of the double stranded target DNA, e.g., the DMPK gene, in its entirety, by the sequence of the RD.

In some embodiments, the RD has a biological function or encodes a polypeptide having a biological function. In some embodiments, the RD comprises an expression cassette. In some embodiments, the RD comprises a nucleotide sequence that encodes an expression tag, for example, an affinity tag, a His tag, a V5 tag, or a FLAG tag. In some embodiments, the RD comprises a nucleotide sequence that encodes a His tag. In some embodiments, the RD comprises a nucleotide sequence that encodes a FLAG tag. In some embodiments, the RD comprises a nucleotide sequence that encodes an attB or an attP sequence. In some embodiments, the RD comprises a nucleotide sequence that encodes a reporter protein, for example, a green fluorescence protein, a blue fluorescence protein, a cyan fluorescence protein, a yellow fluorescence protein, an auto fluorescent protein, or a luciferase. In some embodiments, the RD comprises a recognition site of an enzyme, for example, a recombinase recognition sequence. In some embodiments, the RD comprises a nucleotide sequence that encodes a selectable marker, for example, an antibiotic resistance marker. In some embodiments, the RD comprises a regulatory sequence, for example, a promoter, an enhancer, or an insulator. In some embodiments, the RD comprises a trackable sequence, for example, a barcode. In some embodiments, replacement of the IND by the RD restores or partially restores the function of the target gene. In some embodiments, the target gene is a disease-associated gene. In some embodiments, the target gene is a monogenic disease-associated gene. In some embodiments, the target gene is a polygenic disease-associated gene. In some embodiments, the target gene is a disease-associated gene containing one or more disease-causing mutations, wherein replacement of the IND by the RD corrects the mutations, thereby restoring or partially restoring the function of the target gene. In some embodiments, the disease-associated gene containing one or more disease-causing mutations is in a human subject in need of treatment. In some embodiments, the target gene is a mutated gene causing a disease or disorder in a human subject, wherein replacement of the IND by the RD corrects the mutated gene, thereby restoring or partially restoring the function of the target gene. In some embodiments, the target gene is a disease-associated gene containing one or more disease-causing mutations, wherein replacement of the IND by the RD modifies the target gene to restore or partially restore the function of the target gene. In some embodiments, the disease-associated gene containing one or more disease-causing mutations is in a human subject in need of treatment. In some embodiments, the target gene is a mutated gene causing a disease or disorder in a human subject, wherein replacement of the IND by the RD modifies the mutated gene to restore or partially restore the function of the target gene.

In some embodiments, the first editing template and the second editing template are partially complementary to each other. As used herein, the first editing template is partially complementary to the second editing template when the first and the second editing templates have complementary or substantially complementary region(s) over part of the length of both editing templates. The partially complementary region(s) in the first editing template and the second editing template can be in any position within the first editing template and the second editing template. Accordingly, in some embodiments, the first newly synthesized single stranded DNA encoded by the first editing template and the second newly synthesized single stranded DNA encoded by the second editing template are partially complementary to each other, at any position within the first newly synthesized single stranded DNA encoded by the first editing template and the second newly synthesized single stranded DNA encoded by the second editing template. In some embodiments, the first newly synthesized single stranded DNA comprises a region of complementarity to the second newly synthesized single stranded DNA, at or near the 3′ end of the first newly synthesized single stranded DNA. In some embodiments, the first newly synthesized single stranded DNA comprises a region of complementarity to the second newly synthesized single stranded DNA, at or near the 5′ end of the first newly synthesized single stranded DNA. In some embodiments, the first newly synthesized single stranded DNA comprises a region of complementarity to the second newly synthesized single stranded DNA, in the middle of the first newly synthesized single stranded DNA.

In some embodiments, the second newly synthesized single stranded DNA comprises a region of complementarity to the first newly synthesized single stranded DNA, at or near the 3′ end of the second newly synthesized single stranded DNA. In some embodiments, the second newly synthesized single stranded DNA comprises a region of complementarity to the first newly synthesized single stranded DNA, at or near the 5′ end of the second newly synthesized single stranded DNA. In some embodiments, the second newly synthesized single stranded DNA comprises a region of complementarity to the first newly synthesized single stranded DNA, in the middle of the second newly synthesized single stranded DNA.

In some embodiments, the first newly synthesized single stranded DNA and the second newly synthesized single stranded DNA each comprises a region of complementarity to each other at the 3′ end of each of the first newly synthesized single stranded DNA and the second newly synthesized single stranded DNA.

In some embodiments, the first editing template and the second editing template are of the same length. In some embodiments, the first editing template and the second editing template are of different lengths.

In some embodiments, the first editing template comprises a region that has complementarity or substantial complementarity to the second editing template (the OD encoding region), and further comprises a region that does not have complementarity to the second editing template. In some embodiments, the first editing template comprises a region that has complementarity or substantial complementarity to the second editing template (the OD encoding region), wherein the region is flanked by one or more regions that do not have complementarity to the second editing template. In some embodiments, the entirety of the first editing template has complementarity or substantial complementarity to a region of the second editing template, wherein the second editing template comprises a region that does not have complementarity to the first editing template.

In some embodiments, the first editing template comprises a region that does not have complementarity to the second editing template, wherein the region is about 5 to 10, 5 to 15, 5 to 20, 5 to 25, 5 to 30, 5 to 35, 5 to 40, 5 to 45, 5 to 50, 5 to 55, 5 to 60, 5 to 65, 5 to 70, 5 to 75, 5 to 80, 5 to 85, 5 to 90, 5 to 95, 5 to 100, 5 to 110, 5 to 120, 5 to 130, 5 to 140, 5 to 150, 5 to 175, 5 to 200, 5 to 225, 5 to 250, 5 to 275, 5 to 300, 5 to 325, 5 to 350, 5 to 375, 5 to 400, 5 to 425, 5 to 450, 5 to 475, 5 to 500, 10 to 15, 10 to 20, 10 to 25, 10 to 30, 10 to 35, 10 to 40, 10 to 45, 10 to 50, 10 to 55, 10 to 60, 10 to 65, 10 to 70, 10 to 75, 10 to 80, 10 to 85, 10 to 90, 10 to 95, 10 to 100, 10 to 110, 10 to 120, 10 to 130, 10 to 140, 10 to 150, 10 to 175, 10 to 200, 10 to 225, 10 to 250, 10 to 275, 10 to 300, 10 to 325, 10 to 350, 10 to 375, 10 to 400, 10 to 425, 10 to 450, 10 to 475, 10 to 500, 15 to 20, 15 to 25, 15 to 30, 15 to 35, 15 to 40, 15 to 45, 15 to 50, 15 to 55, 15 to 60, 15 to 65, 15 to 70, 15 to 75, 15 to 80, 15 to 85, 15 to 90, 15 to 95, 15 to 100, 15 to 110, 15 to 120, 15 to 130, 15 to 140, 15 to 150, 15 to 175, 15 to 200, 15 to 225, 15 to 250, 15 to 275, 15 to 300, 15 to 325, 15 to 350, 15 to 375, 15 to 400, 15 to 425, 15 to 450, 15 to 475, 15 to 500, 20 to 25, 20 to 30, 20 to 35, 20 to 40, 20 to 45, 20 to 50, 20 to 55, 20 to 60, 20 to 65, 20 to 70, 20 to 75, 20 to 80, 20 to 85, 20 to 90, 20 to 95, 20 to 100, 20 to 110, 20 to 120, 20 to 130, 20 to 140, 20 to 150, 20 to 175, 20 to 200, 20 to 225, 20 to 250, 20 to 275, 20 to 300, 20 to 325, 20 to 350, 20 to 375, 20 to 400, 20 to 425, 20 to 450, 20 to 475, 20 to 500, 30 to 35, 30 to 40, 30 to 45, 30 to 50, 30 to 55, 30 to 60, 30 to 65, 30 to 70, 30 to 75, 30 to 80, 30 to 85, 30 to 90, 30 to 95, 30 to 100, 30 to 110, 30 to 120, 30 to 130, 30 to 140, 30 to 150, 30 to 175, 30 to 200, 30 to 225, 30 to 250, 30 to 275, 30 to 300, 30 to 325, 30 to 350, 30 to 375, 30 to 400, 30 to 425, 30 to 450, 30 to 475, 30 to 500, 40 to 45, 40 to 50, 40 to 55, 40 to 60, 40 to 65, 40 to 70, 40 to 75, 40 to 80, 40 to 85, 40 to 90, 40 to 95, 40 to 100, 40 to 110, 40 to 120, 40 to 130, 40 to 140, 40 to 150, 40 to 175, 40 to 200, 40 to 225, 40 to 250, 40 to 275, 40 to 300, 40 to 325, 40 to 350, 40 to 375, 40 to 400, 40 to 425, 40 to 450, 40 to 475, 40 to 500, 50 to 55, 50 to 60, 50 to 65, 50 to 70, 50 to 75, 50 to 80, 50 to 85, 50 to 90, 50 to 95, 50 to 100, 50 to 110, 50 to 120, 50 to 130, 50 to 140, 50 to 150, 50 to 175, 50 to 200, 50 to 225, 50 to 250, 50 to 275, 50 to 300, 50 to 325, 50 to 350, 50 to 375, 50 to 400, 50 to 425, 50 to 450, 50 to 475, 50 to 500, 75 to 80, 75 to 85, 75 to 90, 75 to 95, 75 to 100, 75 to 110, 75 to 120, 75 to 130, 75 to 140, 75 to 150, 75 to 175, 75 to 200, 75 to 225, 75 to 250, 75 to 275, 75 to 300, 75 to 325, 75 to 350, 75 to 375, 75 to 400, 75 to 425, 75 to 450, 75 to 475, 75 to 500, 100 to 110, 100 to 120, 100 to 130, 100 to 140, 100 to 150, 100 to 175, 100 to 200, 100 to 225, 100 to 250, 100 to 275, 100 to 300, 100 to 325, 100 to 350, 100 to 375, 100 to 400, 100 to 425, 100 to 450, 100 to 475, 100 to 500, 125 to 150, 125 to 175, 125 to 200, 125 to 225, 125 to 250, 125 to 275, 125 to 300, 125 to 325, 125 to 350, 125 to 375, 125 to 400, 125 to 425, 125 to 450, 125 to 475, 125 to 500, 150 to 175, 150 to 200, 150 to 225, 150 to 250, 150 to 275, 150 to 300, 150 to 325, 150 to 350, 150 to 375, 150 to 400, 150 to 425, 150 to 450, 150 to 475, 150 to 500, 175 to 200, 175 to 225, 175 to 250, 175 to 275, 175 to 300, 175 to 325, 175 to 350, 175 to 375, 175 to 400, 175 to 425, 175 to 450, 175 to 475, 175 to 500, 200 to 250, 200 to 275, 200 to 300, 200 to 325, 200 to 350, 200 to 375, 200 to 400, 200 to 425, 200 to 450, 200 to 475, 200 to 500, 225 to 250, 225 to 275, 225 to 300, 225 to 325, 225 to 350, 225 to 375, 225 to 400, 225 to 425, 225 to 450, 225 to 475, 225 to 500, 250 to 275, 250 to 300, 275 to 300, 275 to 325, 275 to 350, 275 to 375, 275 to 400, 275 to 425, 275 to 450, 275 to 475, 275 to 500, 300 to 325, 300 to 350, 300 to 375, 300 to 400, 300 to 425, 300 to 450, 300 to 475, 300 to 500, 325 to 350, 325 to 375, 325 to 400, 325 to 425, 325 to 450, 325 to 475, 325 to 500, 350 to 375, 350 to 400, 350 to 425, 350 to 450, 350 to 475, 350 to 500, 375 to 400, 375 to 425, 375 to 450, 375 to 475, 375 to 500, 400 to 425, 400 to 450, 400 to 475, 400 to 500, 425 to 450, 425 to 475, 425 to 500, 450 to 475, 450 to 500, or 475 to 500 nucleotides in length. In some embodiments, the first editing template comprises a region that does not have complementarity to the second editing template, wherein the region is about 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 or more nucleotides in length.

In some embodiments, the second editing template comprises a region that has complementarity or substantial complementarity to the first editing template, and further comprises a region that does not have complementarity to the first editing template. In some embodiments, the second editing template comprises a region that has complementarity or substantial complementarity to the first editing template, and is flanked by one or more regions that do not have complementarity to the first editing template. The region(s) in the first editing template and the second editing template may have same or different lengths. In some embodiments, the entirety of the second editing template has complementarity or substantial complementarity to a region of the first editing template, wherein the first editing template comprises a region that does not have complementarity to the second editing template.

In some embodiments, the second editing template comprises a region that does not have complementarity to the first editing template, wherein the region is about 5 to 10, 5 to 15, 5 to 20, 5 to 25, 5 to 30, 5 to 35, 5 to 40, 5 to 45, 5 to 50, 5 to 55, 5 to 60, 5 to 65, 5 to 70, 5 to 75, 5 to 80, 5 to 85, 5 to 90, 5 to 95, 5 to 100, 5 to 110, 5 to 120, 5 to 130, 5 to 140, 5 to 150, 5 to 175, 5 to 200, 5 to 225, 5 to 250, 5 to 275, 5 to 300, 5 to 325, 5 to 350, 5 to 375, 5 to 400, 5 to 425, 5 to 450, 5 to 475, 5 to 500, 10 to 15, 10 to 20, 10 to 25, 10 to 30, 10 to 35, 10 to 40, 10 to 45, 10 to 50, 10 to 55, 10 to 60, 10 to 65, 10 to 70, 10 to 75, 10 to 80, 10 to 85, 10 to 90, 10 to 95, 10 to 100, 10 to 110, 10 to 120, 10 to 130, 10 to 140, 10 to 150, 10 to 175, 10 to 200, 10 to 225, 10 to 250, 10 to 275, 10 to 300, 10 to 325, 10 to 350, 10 to 375, 10 to 400, 10 to 425, 10 to 450, 10 to 475, 10 to 500, 15 to 20, 15 to 25, 15 to 30, 15 to 35, 15 to 40, 15 to 45, 15 to 50, 15 to 55, 15 to 60, 15 to 65, 15 to 70, 15 to 75, 15 to 80, 15 to 85, 15 to 90, 15 to 95, 15 to 100, 15 to 110, 15 to 120, 15 to 130, 15 to 140, 15 to 150, 15 to 175, 15 to 200, 15 to 225, 15 to 250, 15 to 275, 15 to 300, 15 to 325, 15 to 350, 15 to 375, 15 to 400, 15 to 425, 15 to 450, 15 to 475, 15 to 500, 20 to 25, 20 to 30, 20 to 35, 20 to 40, 20 to 45, 20 to 50, 20 to 55, 20 to 60, 20 to 65, 20 to 70, 20 to 75, 20 to 80, 20 to 85, 20 to 90, 20 to 95, 20 to 100, 20 to 110, 20 to 120, 20 to 130, 20 to 140, 20 to 150, 20 to 175, 20 to 200, 20 to 225, 20 to 250, 20 to 275, 20 to 300, 20 to 325, 20 to 350, 20 to 375, 20 to 400, 20 to 425, 20 to 450, 20 to 475, 20 to 500, 30 to 35, 30 to 40, 30 to 45, 30 to 50, 30 to 55, 30 to 60, 30 to 65, 30 to 70, 30 to 75, 30 to 80, 30 to 85, 30 to 90, 30 to 95, 30 to 100, 30 to 110, 30 to 120, 30 to 130, 30 to 140, 30 to 150, 30 to 175, 30 to 200, 30 to 225, 30 to 250, 30 to 275, 30 to 300, 30 to 325, 30 to 350, 30 to 375, 30 to 400, 30 to 425, 30 to 450, 30 to 475, 30 to 500, 40 to 45, 40 to 50, 40 to 55, 40 to 60, 40 to 65, 40 to 70, 40 to 75, 40 to 80, 40 to 85, 40 to 90, 40 to 95, 40 to 100, 40 to 110, 40 to 120, 40 to 130, 40 to 140, 40 to 150, 40 to 175, 40 to 200, 40 to 225, 40 to 250, 40 to 275, 40 to 300, 40 to 325, 40 to 350, 40 to 375, 40 to 400, 40 to 425, 40 to 450, 40 to 475, 40 to 500, 50 to 55, 50 to 60, 50 to 65, 50 to 70, 50 to 75, 50 to 80, 50 to 85, 50 to 90, 50 to 95, 50 to 100, 50 to 110, 50 to 120, 50 to 130, 50 to 140, 50 to 150, 50 to 175, 50 to 200, 50 to 225, 50 to 250, 50 to 275, 50 to 300, 50 to 325, 50 to 350, 50 to 375, 50 to 400, 50 to 425, 50 to 450, 50 to 475, 50 to 500, 75 to 80, 75 to 85, 75 to 90, 75 to 95, 75 to 100, 75 to 110, 75 to 120, 75 to 130, 75 to 140, 75 to 150, 75 to 175, 75 to 200, 75 to 225, 75 to 250, 75 to 275, 75 to 300, 75 to 325, 75 to 350, 75 to 375, 75 to 400, 75 to 425, 75 to 450, 75 to 475, 75 to 500, 100 to 110, 100 to 120, 100 to 130, 100 to 140, 100 to 150, 100 to 175, 100 to 200, 100 to 225, 100 to 250, 100 to 275, 100 to 300, 100 to 325, 100 to 350, 100 to 375, 100 to 400, 100 to 425, 100 to 450, 100 to 475, 100 to 500, 125 to 150, 125 to 175, 125 to 200, 125 to 225, 125 to 250, 125 to 275, 125 to 300, 125 to 325, 125 to 350, 125 to 375, 125 to 400, 125 to 425, 125 to 450, 125 to 475, 125 to 500, 150 to 175, 150 to 200, 150 to 225, 150 to 250, 150 to 275, 150 to 300, 150 to 325, 150 to 350, 150 to 375, 150 to 400, 150 to 425, 150 to 450, 150 to 475, 150 to 500, 175 to 200, 175 to 225, 175 to 250, 175 to 275, 175 to 300, 175 to 325, 175 to 350, 175 to 375, 175 to 400, 175 to 425, 175 to 450, 175 to 475, 175 to 500, 200 to 250, 200 to 275, 200 to 300, 200 to 325, 200 to 350, 200 to 375, 200 to 400, 200 to 425, 200 to 450, 200 to 475, 200 to 500, 225 to 250, 225 to 275, 225 to 300, 225 to 325, 225 to 350, 225 to 375, 225 to 400, 225 to 425, 225 to 450, 225 to 475, 225 to 500, 250 to 275, 250 to 300, 275 to 300, 275 to 325, 275 to 350, 275 to 375, 275 to 400, 275 to 425, 275 to 450, 275 to 475, 275 to 500, 300 to 325, 300 to 350, 300 to 375, 300 to 400, 300 to 425, 300 to 450, 300 to 475, 300 to 500, 325 to 350, 325 to 375, 325 to 400, 325 to 425, 325 to 450, 325 to 475, 325 to 500, 350 to 375, 350 to 400, 350 to 425, 350 to 450, 350 to 475, 350 to 500, 375 to 400, 375 to 425, 375 to 450, 375 to 475, 375 to 500, 400 to 425, 400 to 450, 400 to 475, 400 to 500, 425 to 450, 425 to 475, 425 to 500, 450 to 475, 450 to 500, or 475 to 500 nucleotides in length. In some embodiments, the second editing template comprises a region that does not have complementarity to the first editing template, wherein the region is about 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 or more nucleotides in length.

In some embodiments, the RD comprises a region (or a subset) of the sequence of the IND. In some embodiments, the RD consists of a region of the sequence of the IND. In some embodiments, the RD comprises one or more intended nucleotide edits compared to the IND. In some embodiments, the RD comprises a region(s) that has substantial sequence identity to the sequence of the IND, wherein the region(s) comprises one or more nucleotide edits compared to the sequence of the IND. For example, the RD may comprise a region that has substantial sequence identity to the sequence of the IND, wherein the region comprises one or more nucleotide substitutions, insertions, or deletions. In some embodiments, the RD comprises a region of the sequence of the IND, and further comprises a region that does not have sequence identity or complementary to the IND. In some embodiments, the RD comprises a region that has substantial identity to the sequence of the IND comprising one or more nucleotide edits, and further comprises a region that does not have sequence identity or complementary to the IND. In some embodiments, the region that does not have sequence identity or complementary to the IND has a biological function or encodes a polypeptide or a portion thereof having a biological function. In some embodiments, the RD comprises one or more intended nucleotide edits compared to the IND and encodes a polypeptide or a portion thereof.

In some embodiments, the OD comprises a region (or a subset) of the sequence of the IND. In some embodiments, the OD consists of a region of the sequence of the IND. In some embodiments, the OD comprises one or more intended nucleotide edits compared to the IND. In some embodiments, the OD comprises a region(s) that has substantial sequence identity to the sequence of the IND, wherein the region(s) comprise one or more nucleotide edits compared to the sequence of the IND. For example, the OD may comprise a region that has substantial sequence identity to the sequence of the IND, wherein the region comprises one or more nucleotide substitutions, insertions, or deletions. In some embodiments, the OD comprises a region of the sequence of the IND, and further comprises a region that does not have sequence identity or complementary to the IND. In some embodiments, the OD comprises a region that has substantial identity to the sequence of the IND comprising one or more nucleotide edits, and further comprises a region that does not have sequence identity or complementarity to the IND. In some embodiments, the region that does not have sequence identity or complementarity to the IND has a biological function or encodes a polypeptide or a portion thereof having a biological function. In some embodiments, the OD comprises one or more intended nucleotide edits compared to the IND and encodes a polypeptide or a portion thereof.

In some embodiments, the IND comprises an array of nucleotide motifs. In some embodiments, the IND has an array of three nucleotide repeats (or tri-nucleotide repeats). In some embodiments, the IND has an array of 5-10, 5-15, 5-20, 5-25, 5-30, 5-35, 5-40, 5-50, 5-60, 5-70, 5-80, 5-90, 5-100, 20-30, 20-40, 20-50, 20-60, 20-70, 20-80, 20-90, 20-100, 35-40, 35-50, 35-60, 35-70, 35-80, 35-90, 35-100, 50-70, 50-80, 50-90, 50-100, 50-150, 50-200, 50-250, 50-300, 50-350, 50-400, 50-450, 50-500, 50-550, 50-600, 50-650, 50-700, 50-750, 50-800, 50-850, 50-900, 50-950, 50-1000, 50-1050, 50-1100, 50-1150, 50-1200, 50-1250, 50-1300, 50-1350, 50-1400, 50-1450, 50-1500, 100-150, 100-200, 100-250, 100-300, 100-350, 100-400, 100-450, 100-500, 100-550, 100-600, 100-650, 100-700, 100-750, 100-800, 100-850, 100-900, 100-950, 100-1000, 100-1050, 100-1100, 100-1150, 100-1200, 100-1250, 100-1300, 100-1350, 100-1400, 100-1450, 100-1500, 150-200, 150-250, 150-300, 150-350, 150-400, 150-450, 150-500, 150-550, 150-600, 150-650, 150-700, 150-750, 150-800, 150-850, 150-900, 150-950, 150-1000, 150-1050, 150-1100, 150-1150, 150-1200, 150-1250, 150-1300, 150-1350, 150-1400, 150-1450, 150-1500, 200-250, 200-300, 200-350, 200-400, 200-450, 200-500, 200-550, 200-600, 200-650, 200-700, 200-750, 200-800, 200-850, 200-900, 200-950, 200-1000, 200-1050, 200-1100, 200-1150, 200-1200, 200-1250, 200-1300, 200-1350, 200-1400, 200-1450, 200-1500, 250-300, 250-350, 250-400, 250-450, 250-500, 250-550, 250-600, 250-650, 250-700, 250-750, 250-800, 250-850, 250-900, 250-950, 250-1000, 250-1050, 250-1100, 250-1150, 250-1200, 250-1250, 250-1300, 250-1350, 250-1400, 250-1450, 250-1500, 300-350, 300-400, 300-450, 300-500, 300-550, 300-600, 300-650, 300-700, 300-750, 300-800, 300-850, 300-900, 300-950, 300-1000, 300-1050, 300-1100, 300-1150, 300-1200, 300-1250, 300-1300, 300-1350, 300-1400, 300-1450, 300-1500, 400-450, 400-500, 400-550, 400-600, 400-650, 400-700, 400-750, 400-800, 400-850, 400-900, 400-950, 400-1000, 400-1050, 400-1100, 400-1150, 400-1200, 400-1250, 400-1300, 400-1350, 400-1400, 400-1450, 400-1500, 500-550, 500-600, 500-650, 500-700, 500-750, 500-800, 500-850, 500-900, 500-950, 500-1000, 500-1050, 500-1100, 500-1150, 500-1200, 500-1250, 500-1300, 500-1350, 500-1400, 500-1450, 500-1500, 600-650, 600-700, 600-750, 600-800, 600-850, 600-900, 600-950, 600-1000, 600-1050, 600-1100, 600-1150, 600-1200, 600-1250, 600-1300, 600-1350, 600-1400, 600-1450, 600-1500, 700-750, 700-800, 700-850, 700-900, 700-950, 700-1000, 700-1050, 700-1100, 700-1150, 700-1200, 700-1250, 700-1300, 700-1350, 700-1400, 700-1450, 700-1500, 800-850, 800-900, 800-950, 800-1000, 800-1050, 800-1100, 800-1150, 800-1200, 800-1250, 800-1300, 800-1350, 800-1400, 800-1450, 800-1500, 900-950, 900-1000, 900-1050, 900-1100, 900-1150, 900-1200, 900-1250, 900-1300, 900-1350, 900-1400, 900-1450, 900-1500, 1000-1050, 1000-1100, 1000-1150, 1000-1200, 1000-1250, 1000-1300, 1000-1350, 1000-1400, 1000-1450, 1000-1500, 1100-1200, 1100-1300, 1100-1400, 1100-1500, 1200-1300, 1200-1400, 1200-1500, 1300-1400, 1300-1500, or 1400-1500 tri-nucleotide repeats. In some embodiments, the IND has an array of more than 100, 200, 300, 400, 500, 600, 700, 800, 900, or 1000 repeats. In some embodiments, the IND has an array of more than 1000 repeats. In some embodiments, the IND has an array of more than 1500 repeats. In some embodiments, the IND has an array of 35-50 CTG repeats. In some embodiments, the IND has an array of 35-49 CTG repeats. In some embodiments, the IND has an array of 50-1000 CTG repeats. In some embodiments, the IND has an array of 100-1000 CTG repeats. In some embodiments, the IND has an array of 50-150 CTG repeats. In some embodiments, the IND has an array of more than 1000 CTG repeats.

In some embodiments, the first editing template comprises a region of identity to a sequence adjacent to the second nick site on the second PAM strand of the double stranded target DNA, wherein the sequence is outside the IND. In some embodiments, the second editing template comprises a region of identity to a sequence adjacent to the first nick site on the first PAM strand of the double stranded target DNA, wherein the sequence is outside the IND. Accordingly, in some embodiments, the first newly synthesized single stranded DNA encoded by the first editing template comprises a region of complementarity to a sequence adjacent to the second nick site on the second PAM strand of the double stranded target DNA, wherein the sequence is outside the IND. In some embodiments, the second newly synthesized single stranded DNA encoded by the second editing template comprises a region of complementarity to a sequence adjacent to the first nick site on the first PAM strand of the double stranded target DNA, wherein the sequence is outside the IND.

In some embodiments, the first newly synthesized single stranded DNA comprises a region of complementarity to a sequence immediately adjacent to the second nick site on the second PAM strand of the double stranded target DNA, wherein the sequence is outside the IND. In some embodiments, the second newly synthesized single stranded DNA encoded by the second editing template comprises a region of complementarity to a sequence immediately adjacent to the first nick site on the first PAM strand of the double stranded target DNA, wherein the sequence is outside the IND (see, e.g., FIG. 4F).

In some embodiments, the first newly synthesized single stranded DNA comprises a region of complementarity to a sequence adjacent to the second nick site on the second PAM strand of the double stranded target DNA, wherein the sequence is outside the IND, and is 1, 2, 3, 4, 5, 6, 7, 8, 9, or 10 nucleotides apart from the second nick site. In some embodiments, the second newly synthesized single stranded DNA encoded by the second editing template comprises a region of complementarity to a sequence adjacent to the first nick site on the first PAM strand of the double stranded target DNA, wherein the sequence is outside the IND, and is 1, 2, 3, 4, 5, 6, 7, 8, 9, or 10 nucleotides apart from the first nick site.

In some embodiments, the IND consists of all tri-nucleotide repeats of the double stranded target DNA, e.g. the DMPK gene. In some embodiments, through prime editing and DNA repair, the IND is excised, and the tri-nucleotide repeats are deleted from the double stranded target DNA, e.g., the DMPK gene.

In some embodiments, the IND comprises the tri-nucleotide repeats of the double stranded target DNA, and further comprises one or more base pairs upstream and/or downstream of the tri-nucleotide repeats of the double stranded target DNA, e.g., the DMPK gene. In some embodiments, through prime editing and DNA repair, the IND is excised, and the array of tri-nucleotide repeats, along with the one or more base pairs upstream and/or downstream of the array of tri-nucleotide repeats are deleted from the double stranded target DNA, e.g., the DMPK gene. Accordingly, in some embodiments, incorporation of the first newly synthesized single stranded DNA and the second newly synthesized single stranded DNA results in incorporation of one or more intended nucleotide edits, which comprise deletion of the array of the tri-nucleotide repeat sequence. In some embodiments, incorporation of the first newly synthesized single stranded DNA and the second newly synthesized single stranded DNA results incorporation of one or more intended nucleotide edits, which comprise deletion of the array of the tri-nucleotide repeat sequence and deletion of the one or more base pairs upstream and/or downstream of the array of trinucleotide repeat sequence.

In some embodiments, the first editing template and the second editing template each comprises a region of complementarity or substantial complementarity to each other. In some embodiments, the first editing template comprises a sequence that is exogenous to the double stranded target DNA. In some embodiments, the second editing template comprise a sequence that is exogenous to the double stranded target DNA. In some embodiments, the sequence in the first editing template that is exogenous to the double stranded target DNA comprises a region of complementarity or substantial complementarity to the sequence in the second editing template that is exogenous to the double stranded target DNA. In some embodiments, the sequence in the first editing template that is exogenous to the double stranded target DNA further comprises a region that is not complementary to the sequence in the second editing template that is exogenous to the double stranded target DNA. In some embodiments, the sequence in the second editing template that is exogenous to the double stranded target DNA comprises a region of complementarity or substantial complementarity to the sequence in the first editing template that is exogenous to the double stranded target DNA. In some embodiments, the sequence in the second editing template that is exogenous to the double stranded target DNA further comprises a region that is not complementary to the sequence in the first editing template that is exogenous to the double stranded target DNA. In some embodiments, the first editing template comprises a sequence exogenous to the double stranded target DNA, wherein the sequence exogenous to the double stranded target DNA comprises a polynucleotide sequence that encodes an expression tag, for example, an affinity tag, a His tag, a V5 tag, or a FLAG tag. In some embodiments, the first editing template comprises a sequence exogenous to the double stranded target DNA, wherein the sequence exogenous to the double stranded target DNA comprises an attB or an attP sequence. In some embodiments, the second editing template comprises a sequence exogenous to the double stranded target DNA, wherein the sequence exogenous to the double stranded target DNA comprises a polynucleotide sequence that encodes an expression tag, for example, an affinity tag, a His tag, a V5 tag, or a FLAG tag.

Accordingly, in some embodiments, the first newly synthesized single stranded DNA and the second newly synthesized single stranded DNA each comprises a region of complementarity or substantial complementarity to each other. In some embodiments, the first newly synthesized single stranded DNA comprise a sequence that is exogenous to the double stranded target DNA. In some embodiments, the second newly synthesized single stranded DNA comprise a sequence that is exogenous to the double stranded target DNA. In some embodiments, the sequence in the first newly synthesized single stranded DNA that is exogenous to the double stranded target DNA comprises a region of complementarity or substantial complementarity to the sequence in the second newly synthesized single stranded DNA that is exogenous to the double stranded target DNA. In some embodiments, the sequence in the first newly synthesized single stranded DNA that is exogenous to the double stranded target DNA further comprises a region that is not complementary to the sequence in the second newly synthesized single stranded DNA that is exogenous to the double stranded target DNA. In some embodiments, the sequence in the second newly synthesized single stranded DNA that is exogenous to the double stranded target DNA comprises a region of complementarity or substantial complementarity to the sequence in the first newly synthesized single stranded DNA that is exogenous to the double stranded target DNA. In some embodiments, the sequence in the second newly synthesized single stranded DNA that is exogenous to the double stranded target DNA further comprises a region that is not complementary to the sequence in the first newly synthesized single stranded DNA that is exogenous to the double stranded target DNA.

Accordingly, in some embodiments, the first newly synthesized single stranded DNA and the second newly synthesized single stranded DNA form an OD that comprises a sequence that is exogenous to the double stranded target DNA, e.g. the DMPK gene. In some embodiments, the first newly synthesized single stranded DNA and the second newly synthesized single stranded DNA form an RD that comprises a sequence that is exogenous to the double stranded target DNA, e.g. the DMPK gene. In some embodiments, the IND comprises substantially all or all tri-nucleotide repeats of the double stranded target DNA, e.g. the DMPK gene. Through prime editing, in some embodiments, the IND is excised and is replaced by the RD. In some embodiments, the IND is excised and is replaced by the RD. Accordingly, in some embodiments, substantially all or all tri-nucleotide repeats of the double stranded target DNA, e.g., the DMPK gene, are deleted and replaced by the sequence exogenous to the double stranded target DNA, e.g., the DMPK gene. In some embodiments, the sequence exogenous to the double stranded target DNA comprises a polynucleotide sequence that encodes an expression tag, for example, an affinity tag, a His tag, a V5 tag, or a FLAG tag. In some embodiments, the sequence exogenous to the double stranded target DNA comprises an attB or an attP sequence. Accordingly, in some embodiments, incorporation of the one or more intended nucleotide edits comprises deletion of array of the tri-nucleotide repeat sequence and incorporation of one or more exogenous sequences encoded by the first editing template and/or the second editing template.

In some embodiments, the first editing template comprises a sequence that has complementarity or substantial complementarity to an endogenous sequence of the double stranded target DNA, e.g., the DMPK gene. In some embodiments, the second editing template comprises a sequence that has complementarity or substantial complementarity to an endogenous sequence of the double stranded target DNA, e.g., the DMPK gene. In some embodiments, the first and/or the second editing template comprises a sequence that has complementarity or substantial complementarity to an endogenous sequence of the double stranded target gene, e.g., the DMPK gene, wherein the endogenous sequence of the double stranded target DNA, e.g., the DMPK gene does not comprise the array of CTG tri-nucleotide repeat of the double stranded target DNA, e.g., the DMPK gene. In some embodiments, the endogenous sequence of the double stranded target DNA, e.g., the DMPK gene, that has complementarity or substantial complementarity to the first and/or the second editing template does not comprise an array of tri-nucleotide repeat or any nucleotide repeat structure. In some embodiments, the first editing template comprises a sequence that has complementarity or substantial complementarity to an endogenous sequence on the second strand of the double stranded target DNA, e.g., the DMPK gene, wherein the endogenous sequence of the double stranded target DNA, e.g., the DMPK gene, is upstream of the array of tri-nucleotide repeats. In some embodiments, the first editing template comprises a sequence that has complementarity or substantial complementarity to an endogenous sequence on the second strand of the double stranded target DNA, e.g., the DMPK gene, wherein the endogenous sequence of the double stranded target DNA, e.g., the DMPK gene, is downstream of the array of tri-nucleotide repeats.

In some embodiments, the second editing template comprises a sequence that has complementarity or substantial complementarity to an endogenous sequence on the first strand of the double stranded target DNA, e.g., the DMPK gene, wherein the endogenous sequence of the double stranded target DNA, e.g., the DMPK gene, is upstream of the array of tri-nucleotide repeats. In some embodiments, the second editing template comprises a sequence that has complementarity or substantial complementarity to an endogenous sequence on the first strand of the double stranded target DNA, e.g., the DMPK gene, wherein the endogenous sequence of the double stranded target DNA, e.g., the DMPK gene, is downstream of the array of tri-nucleotide repeats.

In some embodiments, the first editing template comprises a sequence that has complementarity or substantial complementarity to an endogenous sequence on the second strand of the double stranded target DNA that is upstream of the array of the trinucleotide-repeats, wherein the endogenous sequence is 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleotides in length.

In some embodiments, the first editing template comprises a sequence that has complementarity or substantial complementarity to an endogenous sequence on the second strand of the double stranded target DNA that is downstream of the array of the trinucleotide-repeats, wherein the endogenous sequence is 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleotides in length.

In some embodiments, the first editing template comprises a sequence that has complementarity or substantial complementarity to an endogenous sequence on the second strand of the double stranded target DNA that is upstream of the array of the trinucleotide-repeats, wherein the endogenous sequence is 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 250, 300, 350, 400, 450, 500 or more nucleotides upstream of the array of the tri-nucleotide repeats as measured at the 5′ ends. In some embodiments, the first editing template comprises a sequence that has complementarity or substantial complementarity to an endogenous sequence on the second strand of the double stranded target DNA that is downstream of the array of the trinucleotide-repeats, wherein the endogenous sequence is 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 250, 300, 350, 400, 450, 500 or more nucleotides downstream of the array of the tri-nucleotide repeats as measured at the 5′ ends.

In some embodiments, the second editing template comprises a sequence that has complementarity or substantial complementarity to an endogenous sequence on the first strand of the double stranded target DNA that is upstream of the array of the trinucleotide-repeats, wherein the endogenous sequence is 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleotides in length.

In some embodiments, the second editing template comprises a sequence that has complementarity or substantial complementarity to an endogenous sequence on the first strand of the double stranded target DNA that is downstream of the array of the trinucleotide-repeats, wherein the endogenous sequence is 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleotides in length.

In some embodiments, the second editing template comprises a sequence that has complementarity or substantial complementarity to an endogenous sequence on the first strand of the double stranded target DNA that is upstream of the array of the trinucleotide-repeats, wherein the endogenous sequence is 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 250, 300, 350, 400, 450, 500 or more nucleotides upstream of the array of the tri-nucleotide repeats as measured at the 5′ ends.

In some embodiments, the second editing template comprises a sequence that has complementarity or substantial complementarity to an endogenous sequence on the first strand of the double stranded target DNA that is downstream of the array of the trinucleotide-repeats, wherein the endogenous sequence is 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 250, 300, 350, 400, 450, 500 or more nucleotides downstream of the array of the tri-nucleotide repeats as measured at the 5′ ends.

In some embodiments, the sequence of the first editing template that has complementarity or substantial complementarity to an endogenous sequence of the double stranded target DNA comprises a region of complementarity or substantial complementarity to the sequence of the second editing template that has complementarity or substantial complementarity to an endogenous sequence of the double stranded target DNA. In some embodiments, the sequence of the first editing template that complementarity or substantial complementarity to an endogenous sequence of the double stranded target DNA further comprises a region that is not complementary to the sequence of the second editing template that has complementarity or substantial complementarity to an endogenous sequence of the double stranded target DNA. In some embodiments, the sequence of the second editing template that has complementarity or substantial complementarity to an endogenous sequence of the double stranded target DNA comprises a region of complementarity or substantial complementarity to the sequence of the first editing template that has complementarity or substantial complementarity to an endogenous sequence of the double stranded target DNA. In some embodiments, the sequence of the second editing template that has complementarity or substantial complementarity to an endogenous sequence of the double stranded target DNA further comprises a region that is not complementary to the sequence of the first editing template that has identity or substantial identity to an endogenous sequence of the double stranded target DNA.

Accordingly, in some embodiments, the first newly synthesized single stranded DNA comprises a sequence that has identity or substantial identity to an endogenous sequence of the double stranded target DNA, e.g., the DMPK gene. In some embodiments, the second newly synthesized single stranded DNA comprises a sequence that has identity or substantial identity to an endogenous sequence of the double stranded target DNA, e.g., the DMPK gene. In some embodiments, the first newly synthesized single stranded DNA and/or the second newly synthesized single stranded DNA comprises a sequence that has identity or substantial identity to an endogenous sequence of the double stranded target DNA, e.g., the DMPK gene, wherein the endogenous sequence of the double stranded target DNA, e.g., the DMPK gene does not comprise the array of tri-nucleotide repeat of the double stranded target DNA, e.g., the DMPK gene. In some embodiments, the first newly synthesized single stranded DNA comprises a sequence that has identity or substantial identity to an endogenous sequence on the second strand of the double stranded target DNA, e.g., the DMPK gene, wherein the endogenous sequence of the double stranded target DNA, e.g., the DMPK gene, is upstream of the array of tri-nucleotide repeats. In some embodiments, the first newly synthesized single stranded DNA comprises a sequence that has identity or substantial identity to an endogenous sequence on the second strand of the double stranded target DNA, e.g., the DMPK gene, wherein the endogenous sequence of the double stranded target DNA, e.g., the DMPK gene, is downstream of the array of tri-nucleotide repeats. In some embodiments, the second newly synthesized single stranded DNA comprises a sequence that has identity or substantial identity to an endogenous sequence on the first strand of the double stranded target DNA, e.g., the DMPK gene, wherein the endogenous sequence of the double stranded target DNA, e.g., the DMPK gene, is upstream of the array of tri-nucleotide repeats. In some embodiments, the second newly synthesized single stranded DNA comprises a sequence that has identity or substantial identity to an endogenous sequence on the first strand of the double stranded target DNA, e.g., the DMPK gene, wherein the endogenous sequence of the double stranded target DNA, e.g., the DMPK gene, is downstream of the array of tri-nucleotide repeats.

In some embodiments, the array of tri-nucleotide repeats of the DMPK gene is an array of CTG repeats on the coding strand (the second strand) or the reverse complement CAG repeats on the non-coding strand (the first strand).

In some embodiments, the sequence of the first newly synthesized single stranded DNA that has identity or substantial identity to an endogenous sequence of the double stranded target DNA comprises a region of complementarity or substantial complementarity to the sequence of the second newly synthesized single stranded DNA that has identity or substantial identity to an endogenous sequence of the double stranded target DNA. In some embodiments, the sequence of the first newly synthesized single stranded DNA that has identity or substantial identity to an endogenous sequence of the double stranded target DNA further comprises a region that is not complementary to the sequence of the second newly synthesized single stranded DNA that has identity or substantial identity to an endogenous sequence of the double stranded target DNA. In some embodiments, the sequence of the second newly synthesized single stranded DNA that has identity or substantial identity to an endogenous sequence of the double stranded target DNA comprises a region of complementarity or substantial complementarity to the sequence of the first newly synthesized single stranded DNA that has identity or substantial identity to an endogenous sequence of the double stranded target DNA. In some embodiments, the sequence of the second newly synthesized single stranded DNA that has identity or substantial identity to an endogenous sequence of the double stranded target DNA further comprises a region that is not complementary to the sequence of the first newly synthesized single stranded DNA that has identity or substantial identity to an endogenous sequence of the double stranded target DNA.

Accordingly, in some embodiments, the first newly synthesized single stranded DNA and the second newly synthesized single stranded DNA form an OD that comprises an endogenous sequence of the double stranded target DNA, e.g. the DMPK gene. In some embodiments, the first newly synthesized single stranded DNA and the second newly synthesized single stranded DNA form an RD that comprises an endogenous sequence of the double stranded target DNA, e.g. the DMPK gene. In some embodiments, the RD or the OD comprises an endogenous sequence of the double stranded target DNA, e.g., the DMPK gene, that is upstream of the array of tri-nucleotide repeats. In some embodiments, the RD or the OD comprises an endogenous sequence of the double stranded target DNA, e.g., the DMPK gene, that is downstream of the array of tri-nucleotide repeats. In some embodiments, the RD or the OD comprises a sequence that is endogenous compared to the double stranded target DNA, e.g., the DMPK gene, wherein the sequence comprises two regions: a) a region that is identical or substantially identical to an endogenous sequence upstream of the array of the tri-nucleotide repeats, and b) a region that is identical or substantially identical to an endogenous sequence downstream of the array of the tri-nucleotide repeats. In some embodiments, the region identical or substantially identical to the endogenous sequence upstream of the array of the trinucleotide-repeats is 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 base pairs in length. In some embodiments, the region identical or substantially identical to the endogenous sequence downstream of the array of the trinucleotide-repeats is 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 base pairs in length. In some embodiments, the region identical or substantially identical to the endogenous sequence upstream of the array of the trinucleotide-repeats is 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 250, 300, 350, 400, 450, 500 or more base pairs upstream of the array of the tri-nucleotide repeats as measured at the 5′ ends. In some embodiments, the region identical or substantially identical to the endogenous sequence upstream of the array of the trinucleotide-repeats is 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 250, 300, 350, 400, 450, 500 or more base pairs downstream of the array of the tri-nucleotide repeats as measured at the 5′ ends. In some embodiments, the array of tri-nucleotide repeats of the DMPK gene is an array of CTG (or CAG) repeats.

In some embodiments, the IND comprises all tri-nucleotide repeats of the double stranded target DNA, e.g. the entire array of CTG (or the reverse complement CAG) repeats of the DMPK gene. In some embodiments, the IND comprises all tri-nucleotide repeats of the double stranded target DNA, e.g. the entire array of CTG (or CAG) repeats of the DMPK gene, and further comprises one or more base pairs upstream and/or downstream of the array of tri-nucleotide repeats. Through prime editing, the IND is excised and is replaced by the RD or the OD. Accordingly, in some embodiments, all tri-nucleotide repeats of the double stranded target DNA, e.g., the entire array of CTG (or CAG) repeats of the DMPK gene, are deleted, and the endogenous sequence upstream of the array of tri-nucleotide repeats is retained. In some embodiments, all tri-nucleotide repeats of the double stranded target DNA, e.g. the entire array of CTG (or CAG) repeats of the DMPK gene are deleted, and the endogenous sequence downstream of the array of tri-nucleotide repeats is retained. In some embodiments, the IND comprises all tri-nucleotide repeats of the double stranded target DNA, e.g. the DMPK gene. In some embodiments, the first editing template has a different number of tri-nucleotide repeats compared to the number of tri-nucleotide repeats in the IND. In some embodiments, the first editing template has a reduced number of tri-nucleotide repeats compared to the number of tri-nucleotide repeats in the IND. In some embodiments, the second editing template has a different number of tri-nucleotide repeats compared to the number of tri-nucleotide repeats in the IND. In some embodiments, the second editing template has a reduced number of tri-nucleotide repeats compared to the number of tri-nucleotide repeats in the IND.

Accordingly, in some embodiments, the first newly synthesized single stranded DNA encoded by the first editing template has a different number of tri-nucleotide repeats compared to the number of tri-nucleotide repeats in the IND. In some embodiments, the first newly synthesized single stranded DNA encoded by the first editing template has a reduced number of tri-nucleotide repeats compared to the number of tri-nucleotide repeats in the IND. In some embodiments, the second newly synthesized single stranded DNA encoded by the second editing template has a different number of tri-nucleotide repeats compared to the number of tri-nucleotide repeats in the IND. In some embodiments, the second newly synthesized single stranded DNA encoded by the second editing template has a reduced number of tri-nucleotide repeats compared to the number of tri-nucleotide repeats in the IND. In some embodiments, the first newly synthesized single stranded DNA and the second newly synthesized single stranded DNA can form an OD or a RD that comprises a reduced number of tri-nucleotide repeats compared to the number of tri-nucleotide repeats in the IND.

In some embodiments, the OD has an array of the same nucleotide repeat motifs, for example, CTG repeats, but of a different number compared to the number of the tri-nucleotide repeats in the IND. In some embodiments, the RD has an array of the same nucleotide repeat motifs, for example, CTG repeats, but of a different number compared to the number of the tri-nucleotide repeats in the IND. In some embodiments, the OD has a reduced number of the tri-nucleotide repeats, e.g., CTG repeats compared to the endogenous number of tri-nucleotide repeats of the double stranded target DNA, e.g., the DMPK gene. In some embodiments, the RD has a reduced number of the tri-nucleotide repeats, e.g., CTG repeats, compared to the endogenous number of tri-nucleotide repeats of the double stranded target DNA, e.g., the DMPK gene. In some embodiments, the RD contains at most 30, 20, 10, or 5 CTG tri-nucleotide repeats. In some embodiments, the RD contains 30, 20, 10, or 5 CTG tri-nucleotide repeats. In some embodiments, the OD contains at most 30, 20, 10, or 5 CTG tri-nucleotide repeats. In some embodiments, the OD contains 30, 20, 10, or 5 CTG tri-nucleotide repeats. In some embodiments, the OD contains 5 CTG repeats. In some embodiments, the RD contains 5 CTG repeats. In some embodiments, the RD or the OD contains the same number of tri-nucleotide repeats as a reference gene, for example, a wild type DMPK gene.

Accordingly, in some embodiments, excision of the IND and incorporation of the RD results in deletion of a portion of the nucleotide repeats sequences of the IND from the double stranded target DNA, e.g., the target gene.

In some embodiments, excision of the IND and incorporation of the OD results in deletion of a portion of the nucleotide repeats sequences of the IND from the double stranded target DNA, e.g., the target gene. In some embodiments, the deletion comprises at least 1, 2, 3, 4, 5, 6, 7, 8, 9, 10, 11, 12, 13, 14, 15, 16, 17, 18, 19, 20 or more tri-nucleotide repeats. In some embodiments, the deletion comprises 1-3, 1-5, 1-10, 1-15, 1-20, 1-25, 1-30, 1-35, 1-40, 1-45, 1-50, 1-55, 1-60, 1-75 or more tri-nucleotide repeats. In some embodiments, the deletion comprises deletion of at least 1, 2, 3, 4, 5, 6, 7, 8, 9, 10, 11, 12, 13, 14, 15, 16, 17, 18, 19, 20 tri-nucleotide repeats from the double stranded target DNA, e.g., the target gene. In some embodiments, the deletion comprises deletion of 1-3, 1-5, 1-10, 1-15, 1-20, 1-25, 1-30, 1-35, 1-40, 1-45, 1-50, 1-55, 1-60, 1-75 or more tri-nucleotide repeats from the double stranded target DNA, e.g., the target gene. In some embodiments, the deletion comprises deletion of 5-10, 5-15, 5-20, 5-25, 5-30, 5-35, 5-40, 5-50, 5-60, 5-70, 5-80, 5-90, 5-100, 20-30, 20-40, 20-50, 20-60, 20-70, 20-80, 20-90, 20-100, 35-40, 35-50, 35-60, 35-70, 35-80, 35-90, 35-100, 50-70, 50-80, 50-90, 50-100, 50-150, 50-200, 50-250, 50-300, 50-350, 50-400, 50-450, 50-500, 50-550, 50-600, 50-650, 50-700, 50-750, 50-800, 50-850, 50-900, 50-950, 50-1000, 50-1050, 50-1100, 50-1150, 50-1200, 50-1250, 50-1300, 50-1350, 50-1400, 50-1450, 50-1500, 100-150, 100-200, 100-250, 100-300, 100-350, 100-400, 100-450, 100-500, 100-550, 100-600, 100-650, 100-700, 100-750, 100-800, 100-850, 100-900, 100-950, 100-1000, 100-1050, 100-1100, 100-1150, 100-1200, 100-1250, 100-1300, 100-1350, 100-1400, 100-1450, 100-1500, 150-200, 150-250, 150-300, 150-350, 150-400, 150-450, 150-500, 150-550, 150-600, 150-650, 150-700, 150-750, 150-800, 150-850, 150-900, 150-950, 150-1000, 150-1050, 150-1100, 150-1150, 150-1200, 150-1250, 150-1300, 150-1350, 150-1400, 150-1450, 150-1500, 200-250, 200-300, 200-350, 200-400, 200-450, 200-500, 200-550, 200-600, 200-650, 200-700, 200-750, 200-800, 200-850, 200-900, 200-950, 200-1000, 200-1050, 200-1100, 200-1150, 200-1200, 200-1250, 200-1300, 200-1350, 200-1400, 200-1450, 200-1500, 250-300, 250-350, 250-400, 250-450, 250-500, 250-550, 250-600, 250-650, 250-700, 250-750, 250-800, 250-850, 250-900, 250-950, 250-1000, 250-1050, 250-1100, 250-1150, 250-1200, 250-1250, 250-1300, 250-1350, 250-1400, 250-1450, 250-1500, 300-350, 300-400, 300-450, 300-500, 300-550, 300-600, 300-650, 300-700, 300-750, 300-800, 300-850, 300-900, 300-950, 300-1000, 300-1050, 300-1100, 300-1150, 300-1200, 300-1250, 300-1300, 300-1350, 300-1400, 300-1450, 300-1500, 400-450, 400-500, 400-550, 400-600, 400-650, 400-700, 400-750, 400-800, 400-850, 400-900, 400-950, 400-1000, 400-1050, 400-1100, 400-1150, 400-1200, 400-1250, 400-1300, 400-1350, 400-1400, 400-1450, 400-1500, 500-550, 500-600, 500-650, 500-700, 500-750, 500-800, 500-850, 500-900, 500-950, 500-1000, 500-1050, 500-1100, 500-1150, 500-1200, 500-1250, 500-1300, 500-1350, 500-1400, 500-1450, 500-1500, 600-650, 600-700, 600-750, 600-800, 600-850, 600-900, 600-950, 600-1000, 600-1050, 600-1100, 600-1150, 600-1200, 600-1250, 600-1300, 600-1350, 600-1400, 600-1450, 600-1500, 700-750, 700-800, 700-850, 700-900, 700-950, 700-1000, 700-1050, 700-1100, 700-1150, 700-1200, 700-1250, 700-1300, 700-1350, 700-1400, 700-1450, 700-1500, 800-850, 800-900, 800-950, 800-1000, 800-1050, 800-1100, 800-1150, 800-1200, 800-1250, 800-1300, 800-1350, 800-1400, 800-1450, 800-1500, 900-950, 900-1000, 900-1050, 900-1100, 900-1150, 900-1200, 900-1250, 900-1300, 900-1350, 900-1400, 900-1450, 900-1500, 1000-1050, 1000-1100, 1000-1150, 1000-1200, 1000-1250, 1000-1300, 1000-1350, 1000-1400, 1000-1450, 1000-1500, 1100-1200, 1100-1300, 1100-1400, 1100-1500, 1200-1300, 1200-1400, 1200-1500, 1300-1400, 1300-1500, or 1400-1500 tri-nucleotide repeats from the double stranded target DNA, e.g., the target gene. In some embodiments, the deletion comprises deletion of 100, 200, 300, 400, 500, 600, 700, 800, 900, or 1000 trinucleotide repeats from the double stranded target DNA, e.g., the target gene. In some embodiments, the deletion comprises deletion of more than 1000 trinucleotide repeats from the double stranded target DNA, e.g., the target gene.

In some embodiments, the first editing template has a reduced number of tri-nucleotide repeats compared to the number of tri-nucleotide repeats in the IND, and further comprises a region that is complementary or substantially complementary to a sequence on the second strand of the double stranded target DNA that is downstream of the array of tri-nucleotide repeats. In some embodiments, the first editing template has a reduced number of tri-nucleotide repeats compared to the number of tri-nucleotide repeats in the IND, and further comprises a) a region that is complementary or substantially complementary to a sequence on the second strand of the double stranded target DNA that is downstream of the array of tri-nucleotide repeats, and b) a region that is complementary or substantially complementary to a sequence on the second strand of the double stranded target DNA that is upstream of the array of tri-nucleotide repeats.

In some embodiments, the second editing template has a reduced number of tri-nucleotide repeats compared to the number of tri-nucleotide repeats in the IND, and further comprises a region that is complementary or substantially complementary to a sequence on the first strand of the double stranded target DNA that is upstream of the array of tri-nucleotide repeats. In some embodiments, the second editing template has a reduced number of tri-nucleotide repeats compared to the number of tri-nucleotide repeats in the IND, and further comprises a) a region that is complementary or substantially complementary to a sequence on the first strand of the double stranded target DNA that is upstream of the array of tri-nucleotide repeats, and b) a region that is complementary or substantially complementary to a sequence on the first strand of the double stranded target DNA that is downstream of the array of tri-nucleotide repeats.

Accordingly, in some embodiments, the first newly synthesized single stranded DNA encoded by the first editing template has a reduced number of tri-nucleotide repeats compared to the number of tri-nucleotide repeats in the IND, and further comprises a region that is identical or substantially identical to a sequence on the second strand of the double stranded target DNA that is downstream of the array of tri-nucleotide repeats. In some embodiments, the first newly synthesized single stranded DNA encoded by the first editing template has a reduced number of tri-nucleotide repeats compared to the number of tri-nucleotide repeats in the IND, and further comprises a) a region that is identical or substantially identical to a sequence on the second strand of the double stranded target DNA that is downstream of the array of tri-nucleotide repeats, and b) a region that is identical or substantially identical to a sequence on the second strand of the double stranded target DNA that is upstream of the array of tri-nucleotide repeats.

In some embodiments, the second newly synthesized single stranded DNA encoded by the second editing template has a reduced number of tri-nucleotide repeats compared to the number of tri-nucleotide repeats in the IND, and further comprises a region that is identical or substantially identical to a sequence on the first strand of the double stranded target DNA that is upstream of the array of tri-nucleotide repeats. In some embodiments, the second newly synthesized single stranded DNA encoded by the second editing template has a reduced number of tri-nucleotide repeats compared to the number of tri-nucleotide repeats in the IND, and further comprises a) a region that is identical or substantially identical to a sequence on the first strand of the double stranded target DNA that is upstream of the array of tri-nucleotide repeats, and b) a region that is identical or substantially identical to a sequence on the first strand of the double stranded target DNA that is downstream of the array of tri-nucleotide repeats.

Accordingly, in some embodiments, the RD or the OD has a reduced number of tri-nucleotide repeats compared to the number of tri-nucleotide repeats in the IND, and further comprises a double stranded sequence of the double stranded target DNA that is upstream or downstream of the IND. In some embodiments, the RD or the OD has a reduced number of tri-nucleotide repeats compared to the number of tri-nucleotide repeats in the IND, and further comprises a double stranded sequence of the double stranded target DNA that is upstream of the IND, and a double stranded sequence of the double stranded target DNA that is downstream of the IND.

In some embodiments, through prime editing, the IND is removed, and the sequence of the RD or the OD is incorporated into the double stranded target DNA. As a result, a portion of the trinucleotide repeats of the double stranded target DNA is deleted from the double stranded target DNA, e.g., the target gene, and the sequences flanking the array of tri-nucleotide repeats are retained.

In some embodiments, the first editing template and/or the second editing template is partially complementary, substantially complementary, or identical to the sequence of the IND. In some embodiments, for example, the first editing template comprises a region that is complementary or identical to a region of a sequence of the IND. In some embodiments, the first editing template comprises a region of complementarity to the sequence on the first PAM strand of the IND. In some embodiments, the first editing template further comprises a region of complementarity to the second editing template. In some embodiments, the first editing template is partially complementary, substantially complementary or identical to a sequence of the IND, and is also substantially complementary to the second editing template. In some embodiments, the second editing template comprises a region that is complementary or identical to a region of a sequence of the IND. In some embodiments, the second editing template comprises a region of complementarity to the sequence on the second PAM strand of the IND. In some embodiments, the second editing template further comprises a region of complementarity to the first editing template. In some embodiments, the second editing template is partially complementary, substantially complementary or identical to a sequence of the IND, and is also substantially complementary to the first editing template. In some embodiments, the first editing template and the second editing template each comprises a region of complementarity to a sequence of the IND.

The partially complementary region(s) in the first editing template and the second editing template can be in any position within the first editing template and the second editing template. Accordingly, in some embodiments, the first newly synthesized single stranded DNA encoded by the first editing template and the second newly synthesized single stranded DNA encoded by the second editing template are partially complementary to each other, at any position within the first newly synthesized single stranded DNA encoded by the first editing template and the second newly synthesized single stranded DNA encoded by the second editing template. In some embodiments, the first newly synthesized single stranded DNA comprises a region of complementarity to the first strand of the IND, at or near the 3′ end of the first newly synthesized single stranded DNA. In some embodiments, the first newly synthesized single stranded DNA comprises a region of complementarity to the first strand of the IND, at or near the 5′ end of the first newly synthesized single stranded DNA. In some embodiments, the first newly synthesized single stranded DNA comprises a region of complementarity to the first strand of the IND, in the middle of the first newly synthesized single stranded DNA. In some embodiments, the second newly synthesized single stranded DNA comprises a region of complementarity to the second strand of the IND, at or near the 3′ end of the second newly synthesized single stranded DNA. In some embodiments, the second newly synthesized single stranded DNA comprises a region of complementarity to the second strand of the IND, at or near the 5′ end of the second newly synthesized single stranded DNA. In some embodiments, the second newly synthesized single stranded DNA comprises a region of complementarity to the second strand of the IND, in the middle of the second newly synthesized single stranded DNA. In some embodiments, the first newly synthesized single stranded DNA and the second newly synthesized single stranded DNA each comprises a region of complementarity to each other at the 3′ end.

Accordingly, as exemplified in FIG. 4C-FIG. 4D, in some embodiments, the first newly synthesized single stranded DNA encoded by the first editing template comprises a region that is identical to a region of the sequence on the first PAM strand of the IND. In some embodiments, the second newly synthesized single stranded DNA encoded by the second editing template comprises a region that is identical to a region of the sequence on the second PAM strand of the IND. In some embodiments, the first newly synthesized single stranded DNA comprises two or more sub regions, each of which is identical to a sub region of the sequence on the first PAM strand of the IND (as exemplified in FIG. 4D). The sub regions on the first newly synthesized single stranded DNA and/or the first PAM strand of the IND may or may not be consecutive. For example, the first newly synthesized single stranded DNA may comprise 2 sub regions each identical to a sub region of the sequence on the first PAM strand of the IND, wherein the two sub regions of the sequence on the first PAM strand of the IND are separated by a region that does not have identity or substantial identity to the first newly synthesized single stranded DNA. In some embodiments, the second newly synthesized single stranded DNA comprises two or more sub regions, each of which is identical to a sub region of the sequence on the second PAM strand of the IND (as exemplified in FIG. 4D). The sub regions on the second newly synthesized single stranded DNA and/or the second PAM strand of the IND may or may not be consecutive. For example, the second newly synthesized single stranded DNA may comprise 2 sub regions each identical to a sub region of the sequence on the second PAM strand of the IND, wherein the two sub regions of the sequence on the second PAM strand of the IND are separated by a region that does not have identity or substantial identity to the second newly synthesized single stranded DNA.

In some embodiments, the region of the sequence on the first PAM strand of the IND and the region of the sequence on the second PAM strand of the IND are complementary to each other. In some embodiments, the first newly synthesized single stranded DNA encoded by the first editing template and the second newly synthesized single stranded DNA encoded by the second editing template are at least partially complementary to each other and can anneal to each other to form an OD. In some embodiments, the first newly synthesized single stranded DNA encoded by the first editing template and the second newly synthesized single stranded DNA encoded by the second editing template are substantially complementary or complementary to each other and can anneal to each other to form an OD. In some embodiments, the IND is excised, and the OD is incorporated in the double stranded target DNA at the place of the IND excision. As a result, the portion in the IND that is not complementary or identical to the first editing template or the second editing template is deleted from the double stranded target DNA. In some embodiments, the deletion is at the 3′ end of the IND. In some embodiments, the deletion is at the 5′ end of the IND. In some embodiments, the deletion is in the middle of the IND.

In some embodiments, the first editing template of the first PEgRNA is at least partially complementary, substantially complementary, at least partially identical, or identical to a sequence of the double stranded target DNA outside the IND. “Outside the IND” refers to sequences or positions of the double stranded target DNA that are not in between the two nick sites generated by the first prime editor and the second prime editor. In some embodiments, the first editing template comprises a region of identity to a sequence outside the IND on the second PAM strand (or the first strand) of the double stranded target DNA. In some embodiments, the first editing template comprises a region of identity to a sequence on the first strand of the double stranded target DNA adjacent to the second nick site generated by the second prime editor complexed with the second PEgRNA, wherein the sequence is outside the IND. In some embodiments, the first editing template comprises a region of identity to a sequence on the first strand of the double stranded target DNA immediately adjacent to the second nick site generated by the second prime editor complexed with the second PEgRNA, wherein the sequence is outside the IND. In some embodiments, the region of identity is at least 10, at least 15, at least 20, at least 25, at least 30, at least 35, at least 40, at least 45, at least 50, at least 55, at least 60, at least 65, at least 70, or at least 75 nucleotides in length. In some embodiments, the region of identity is about 10 to 15, about 10 to 20, about 10 to 25, about 10 to 30, about 10 to 35 about 10 to 40, about 15 to 20, about 15 to 25, about 15 to 30, about 15 to 35, about 15 to 40, about 20 to 25, about 20 to 30, about 20 to 35, about 20 to 40, about 25 to 30, about 25 to 35, about 25 to 40, about 30 to 35, or about 35 to 40 nucleotides in length. In some embodiments, the region of identity is no more than 25, no more than 30, no more than 35, no more than 40, or no more than 45 nucleotides in length. In some embodiments, the first editing template comprises a region of identity to the second spacer. In some embodiments, the first editing template comprises nucleotides m to (n−3) of the second spacer, wherein n is the length of the second spacer, and m is any integer between 1 and (n−12). In some embodiments, n is an integer from 16 to 22. For example, for a 20 nucleotide second spacer, the first editing template can contain nucleotides 1-17, 2-17, 3-17, 4-17, 5-17, 6-17, 7-7, or 8-17 of the second spacer.

Accordingly, as exemplified in FIG. 4E, in some embodiments, the first newly synthesized single stranded DNA encoded by the first editing template comprises a region of complementarity to a sequence on the first strand of the double stranded target DNA adjacent or immediately adjacent to the second nick site generated by the second prime editor complexed with the second PEgRNA. In some embodiments, the first newly synthesized single stranded DNA encoded by the first editing template comprises a region of complementarity to a sequence on the first strand of the double stranded target DNA immediately adjacent to the second nick site generated by the second prime editor complexed with the second PEgRNA, wherein the sequence is outside the IND. In some embodiments, the first newly synthesized single stranded DNA anneals with the sequence on the first strand of the double stranded target DNA adjacent or immediately adjacent to the second nick site generated by the second prime editor. In some embodiments, through DNA repair, the IND is excised and deleted from the double stranded target DNA, e.g., the target gene.

In some embodiments, the second editing template of the second PEgRNA is at least partially complementary, substantially complementary, at least partially identical, or identical to a sequence of the double stranded target DNA outside the IND. In some embodiments, the second editing template of the second PEgRNA comprises a region of identity to a sequence outside the IND on the first PAM strand (or the second strand) of the double stranded target DNA. In some embodiments, the second editing template of the second PEgRNA comprises a region of identity to a sequence on the second strand of the double stranded target DNA adjacent to the first nick site generated by the first prime editor complexed with the first PEgRNA, wherein the sequence is outside the IND. In some embodiments, the second editing template of the second PEgRNA comprises a region of identity to a sequence on the second strand of the double stranded target DNA immediately adjacent to the first nick site generated by the first prime editor complexed with the first PEgRNA, wherein the sequence is outside the IND. In some embodiments, the region of identity is at least 10, at least 15, at least 20, at least 25, at least 30, at least 35, at least 40, at least 45, at least 50, at least 55, at least 60, at least 65, at least 70, or at least 75 nucleotides in length. In some embodiments, the region of identity is about 10 to 15, about 10 to 20, about 10 to 25, about 10 to 30, about 10 to 35 about 10 to 40, about 15 to 20, about 15 to 25, about 15 to 30, about 15 to 35, about 15 to 40, about 20 to 25, about 20 to 30, about 20 to 35, about 20 to 40, about 25 to 30, about 25 to 35, about 25 to 40, about 30 to 35, or about 35 to 40 nucleotides in length. In some embodiments, the region of identity is no more than 25, no more than 30, no more than 35, no more than 40, or no more than 45 nucleotides in length. In some embodiments, the second editing template comprises a region of identity to the second spacer. In some embodiments, the second editing template comprises nucleotides m to (n−3) of the first spacer, wherein n is the length of the first spacer, and m is any integer between 1 and (n−12). In some embodiments, n is an integer from 16 to 22. For example, for a 20 nucleotide first spacer, the second editing template can contain nucleotides 1-17, 2-17, 3-17, 4-17, 5-17, 6-17, 7-7, or 8-17 of the second spacer.

Accordingly, as exemplified in FIG. 4E, in some embodiments, the second newly synthesized single stranded DNA encoded by the second editing template comprises a region of complementarity to a sequence on the second strand of the double stranded target DNA adjacent or immediately adjacent to the first nick site generated by the first prime editor complexed with the first PEgRNA. In some embodiments, the second newly synthesized single stranded DNA encoded by the second editing template comprises a region of complementarity to a sequence on the second strand of the double stranded target DNA adjacent or immediately adjacent to the first nick site generated by the first prime editor complexed with the first PEgRNA, wherein the sequence is outside the IND. In some embodiments, the second newly synthesized single stranded DNA anneals with the sequence on the second strand of the double stranded target DNA adjacent or immediately adjacent to the first nick site generated by the first prime editor. In some embodiments, through DNA repair, the IND is excised and deleted from the double stranded target DNA, e.g., the target gene.

In some embodiments, the first editing template of the first PEgRNA comprises a region at least partially identical to a sequence on the first strand of the double stranded target DNA immediately adjacent to the second nick site generated by the second prime editor complexed with the second PEgRNA, wherein the sequence is outside the IND. In some embodiments, the second editing template of the second PEgRNA comprises a region at least partially identical to a sequence on the second strand of the double stranded target DNA immediately adjacent to the first nick site generated by the first prime editor complexed with the first PEgRNA, wherein the sequence is outside the IND. In some embodiments, the first editing template and the second editing template further comprise a region of complementarity or substantial complementarity to each other.

Accordingly, as exemplified in FIG. 4F, in some embodiments, the first newly synthesized single stranded DNA encoded by the first editing template comprises a region of complementarity to a sequence on the first strand of the double stranded target DNA, wherein the sequence is immediately adjacent to the second nick site generated by the second prime editor complexed with the second PEgRNA and is outside the IND. In some embodiments, the second newly synthesized single stranded DNA encoded by the second editing template comprises a region of complementarity or substantial complementarity to a sequence on the second strand of the double stranded target DNA, wherein the sequence is immediately adjacent to the first nick site generated by the first prime editor complexed with the first PEgRNA and is outside the IND. In some embodiments, the first newly synthesized single stranded DNA encoded by the first editing template and the second newly synthesized single stranded DNA encoded by the second editing template further comprise a region of complementarity or substantial complementarity to each other, and can anneal to each other to form an OD.

In some embodiments, the first newly synthesized single stranded DNA encoded by the first editing template further comprises a region that is not complementary to the second newly synthesized single stranded DNA encoded by the second editing template and does not have complementarity or identity to the double stranded target DNA. In some embodiments, the second newly synthesized single stranded DNA encoded by the second editing template further comprises a region that is not complementary to the first newly synthesized single stranded DNA encoded by the first editing template and does not have complementarity or identity to the double stranded target DNA, e.g., the target gene.

Accordingly, in some embodiments, the RD comprises (i) the OD, (ii) the region of the first newly synthesized single stranded DNA that is not complementary to the second newly synthesized single stranded DNA and does not have complementarity or identity to the double stranded target DNA, and a complementary sequence thereof, and (iii) the region of the second newly synthesized single stranded DNA that is not complementary to the first newly synthesized single stranded DNA and does not have complementarity or identity to the double stranded target DNA, and a complementary sequence thereof. In some embodiments, through DNA repair, the IND is excised from the double stranded target DNA, e.g., the DMPK gene, and the RD is incorporated into the double stranded target DNA.

In some embodiments, the IND is excised and deleted from the target gene, and the RD is incorporated at the place of excision of the IND. In some embodiments, the IND is excised and deleted from the target gene, and the OD is incorporated at the place of excision of the IND. In some embodiments, the RD comprises a region of identity to an endogenous sequence of the double stranded target DNA. In some embodiments, the OD comprises a region of identity to an endogenous sequence of the double stranded target DNA. In some embodiments, the RD does not have sequence identity to an endogenous sequence of the double stranded target DNA. In some embodiments, the RD is exogenous to the double stranded target DNA, e.g., the target gene. In some embodiments, the RD has a biological function or encodes a polypeptide having a biological function. In some embodiments, the OD does not have sequence identity to an endogenous sequence of the double stranded target DNA. In some embodiments, the OD is exogenous to the double stranded target DNA, e.g., the target gene. In some embodiments, the OD has a biological function or encodes a polypeptide having a biological function.

In some embodiments, the first editing template of the first PEgRNA comprises a region at least partially identical to a sequence of the double stranded target DNA that is outside the IND and is not immediately adjacent to (also referred to as “distal to”) the second nick site on the second PAM strand of the double stranded target DNA. In some embodiments, the first editing template of the first PEgRNA comprises a region of identity to a sequence of double stranded target DNA on the second PAM strand that is outside the IND and is at least 1, 2, 3, 4, 5, 6, 7, 8, 9, 10, 11, 12, 13, 14, 15, 16, 17, 18, 19, 20 or more nucleotides upstream of the second nick site.

In some embodiments, the second editing template of the second PEgRNA comprises a region at least partially identical to a sequence of the double stranded target DNA that is outside the IND and is not immediately adjacent to the first nick site on the first PAM strand of the double stranded target DNA. In some embodiments, the second editing template of the second PEgRNA comprises a region of identity to a sequence of the double stranded target DNA on the first PAM strand that is outside the IND and is at least 1, 2, 3, 4, 5, 6, 7, 8, 9, 10, 11, 12, 13, 14, 15, 16, 17, 18, 19, 20 or more nucleotides upstream of the first nick site.

Accordingly, in some embodiments, the first newly synthesized single stranded DNA encoded by the first editing template comprises a region of complementarity to a sequence of the double stranded target DNA that is outside the IND and is not immediately adjacent (i.e., distal) to the second nick site on the second PAM strand. In some embodiments, the first newly synthesized DNA encoded by the first editing template can anneal with the sequence that is outside the IND and is not immediately adjacent to the second nick site on the second PAM strand of the double stranded target DNA. In some embodiments, the first newly synthesized DNA encoded by the first editing template comprises a region of complementarity to, and can anneal with a sequence of the double stranded target DNA that is outside the IND and is at least 1, 2, 3, 4, 5, 6, 7, 8, 9, 10, 11, 12, 13, 14, 15, 16, 17, 18, 19, 20 or more nucleotides upstream of the second nick site.

In some embodiments, the second newly synthesized single stranded DNA encoded by the second editing template comprises a region of complementarity to a sequence of the first PAM strand of the double stranded target DNA that is outside the IND and is not immediately adjacent to (also referred to as “distal to”) the first nick site on the first PAM strand. In some embodiments, the second newly synthesized DNA encoded by the second editing template can anneal with the sequence that is outside the IND and is not immediately adjacent to the first nick site on the first PAM strand of the double stranded target DNA. In some embodiments, the second newly synthesized DNA encoded by the second editing template comprises a region of complementarity to, and can anneal with a sequence of the double stranded target DNA that is outside the IND and is at least 1, 2, 3, 4, 5, 6, 7, 8, 9, 10, 11, 12, 13, 14, 15, 16, 17, 18, 19, 20 or more nucleotides upstream of the first nick site.

In some embodiments, through DNA repair, the IND is excised and deleted from the double stranded target DNA, e.g., the target gene. In some embodiments, the endogenous sequence of the double stranded target DNA between the 3′ end of the sequence that is outside the IND and is distal to the second nick site on the second PAM strand and the 3′ end of the sequence of the double stranded target DNA that is outside the IND and is distal to the first nick site on the first PAM strand of the double stranded target DNA is excised and deleted from the double stranded target DNA.

In some embodiments, as exemplified in FIG. 4G, the first protospacer is downstream of the second search target sequence. In some embodiments, the first editing template comprises a region that has complementarity or substantial complementarity to the second editing template, and optionally further comprises a region that does not have a complementarity to the second editing template. In some embodiments, the second editing template comprises a region that has complementarity or substantial complementarity to the first editing template, and optionally further comprises a region that does not have complementarity to the first editing template.

### Prime Editor

The term “prime editor (PE)” refers to the polypeptide or polypeptide components involved in prime editing, or any polynucleotide(s) encoding the polypeptide or polypeptide components. In various embodiments, a prime editor includes a polypeptide domain having DNA binding activity and a polypeptide domain having DNA polymerase activity. In some embodiments, the polypeptide domain having DNA binding activity is a polypeptide domain having programmable DNA binding activity. In some embodiments, the prime editor further comprises a polypeptide domain having nuclease activity. In some embodiments, the polypeptide domain having DNA binding activity comprises a nuclease domain or nuclease activity. In some embodiments, the polypeptide domain having nuclease activity comprises a nickase, or a fully active nuclease. As used herein, the term “nickase” refers to a nuclease capable of cleaving only one strand of a double-stranded DNA target. In some embodiments, the prime editor comprises a polypeptide domain that is an inactive nuclease. In some embodiments, the polypeptide domain having programmable DNA binding activity comprises a nucleic acid guided DNA binding domain, for example, a CRISPR-Cas protein, for example, a Cas9 nickase, a Cpf1 nickase, or another CRISPR-Cas nuclease. In some embodiments, the polypeptide domain having DNA polymerase activity comprises a template-dependent DNA polymerase, for example, a DNA-dependent DNA polymerase or an RNA-dependent DNA polymerase. In some embodiments, the DNA polymerase is a reverse transcriptase. In some embodiments, the prime editor comprises additional polypeptides or polypeptide domains involved in prime editing, for example, a polypeptide domain having 5′ endonuclease activity, e.g., a 5′ endogenous DNA flap endonucleases (e.g., FEN1), for helping to drive the prime editing process towards the edited product formation. In some embodiments, the prime editor further comprises an RNA-protein recruitment polypeptide, for example, a MS2 coat protein.

A prime editor may be engineered. In some embodiments, the polypeptide components of a prime editor do not naturally occur in the same organism or cellular environment. In some embodiments, the polypeptide components of a prime editor may be of different origins or from different organisms. In some embodiments, a prime editor comprises a DNA binding domain and a DNA polymerase domain that are derived from different species. In some embodiments, a prime editor comprises a Cas polypeptide and a reverse transcriptase polypeptide that are derived from different species. For example, a prime editor may comprise a S. pyogenes Cas9 polypeptide and a Moloney murine leukemia virus (M-MLV) reverse transcriptase polypeptide.

In some embodiments, polypeptide domains of a prime editor may be fused or linked by a peptide linker to form a fusion protein. In other embodiments, a prime editor comprises one or more polypeptide domains provided in trans as separate proteins, which are capable of being associated to each other through non-peptide linkages or through aptamers or recruitment sequences. For example, a prime editor may comprise a DNA binding domain and a reverse transcriptase domain associated with each other by an RNA-protein recruitment aptamer, e.g., a MS2 aptamer, which may be linked to a PEgRNA. Prime editor polypeptide components may be encoded by one or more polynucleotides in whole or in part. In some embodiments, a single polynucleotide, construct, or vector encodes the prime editor fusion protein. In some embodiments, multiple polynucleotides, constructs, or vectors each encode a polypeptide domain or portion of a domain of a prime editor, or a portion of a prime editor fusion protein. For example, a prime editor fusion protein may comprise an N-terminal portion fused to an intein-N and a C-terminal portion fused to an intein-C, each of which is individually encoded by an AAV vector.

The term “prime editor complex” is used interchangeably with the term “prime editing complex” and refers to a complex comprising one or more prime editor components (e.g., a polypeptide domain having DNA binding activity and a polypeptide domain having DNA polymerase activity) complexed with a PEgRNA.

### Prime Editor Nucleotide Polymerase Domain

In some embodiments, a prime editor comprises a nucleotide polymerase domain, e.g., a DNA polymerase domain. The DNA polymerase domain may be a wild-type DNA polymerase domain, a full-length DNA polymerase protein domain, or may be a functional mutant, a functional variant, or a functional fragment thereof. In some embodiments, the polymerase domain is a template dependent polymerase domain. For example, the DNA polymerase may rely on a template polynucleotide strand, e.g., the editing template sequence, for new strand DNA synthesis. In some embodiments, the prime editor comprises a DNA-dependent DNA polymerase. For example, a prime editor having a DNA-dependent DNA polymerase can synthesize a new single stranded DNA using a PEgRNA editing template that comprises a DNA sequence as a template. In such cases, the PEgRNA is a chimeric or hybrid PEgRNA, and comprises an extension arm comprising a DNA strand. As used herein, an “extension arm” is a polynucleotide portion of a PEgRNA that comprises an editing template and a primer binding site sequence (PBS). In some embodiments, an extension arm further comprises additional components, for example, a 3′ modifier. The chimeric or hybrid PEgRNA may comprise an RNA portion (including the spacer and the gRNA core) and a DNA portion (the extension arm comprising the editing template that includes a strand of DNA).

The DNA polymerases can be wild type polymerases from eukaryotic, prokaryotic, archael, or viral organisms, and/or the polymerases may be modified by genetic engineering, mutagenesis, or directed evolution-based processes. The polymerases can be a T7 DNA polymerase, T5 DNA polymerase, T4 DNA polymerase, Klenow fragment DNA polymerase, DNA polymerase III and the like. The polymerases can be thermostable, and can include Taq, Tne, Tma, Pfu, Tfl, Tth, Stoffel fragment, VENT® and DEEPVENT® DNA polymerases, KOD, Tgo, JDF3, and mutants, variants and derivatives thereof.

In some embodiments, the DNA polymerase is a bacteriophage polymerase, for example, a T4, T7, or phi29 DNA polymerase. In some embodiments, the DNA polymerase is an archaeal polymerase, for example, pol I type archaeal polymerase or a pol II type archaeal polymerase. In some embodiments, the DNA polymerase comprises a thermostable archaeal DNA polymerase. In some embodiments, the DNA polymerase comprises a eubacterial DNA polymerase, for example, Pol I, Pol II, or Pol III polymerase. In some embodiments, the DNA polymerase is a Pol I family DNA polymerase. In some embodiments, the DNA polymerase is a E. coli Pol I DNA polymerase. In some embodiments, the DNA polymerase is a Pol II family DNA polymerase. In some embodiments, the DNA polymerase is a Pyrococcus furiosus (Pfu) Pol II DNA polymerase. In some embodiments, the DNA Polymerase is a Pol IV family DNA polymerase. In some embodiments, the DNA polymerase is a E. coli Pol IV DNA polymerase.

In some embodiments, the DNA polymerase comprises a eukaryotic DNA polymerase. In some embodiments, the DNA polymerase is a Pol-beta DNA polymerase, a Pol-lambda DNA polymerase, a Pol-sigma DNA polymerase, or a Pol-mu DNA polymerase. In some embodiments, the DNA polymerase is a Pol-alpha DNA polymerase. In some embodiments, the DNA polymerase is a POLA1 DNA polymerase. In some embodiments, the DNA polymerase is a POLA2 DNA polymerase. In some embodiments, the DNA polymerase is a Pol-delta DNA polymerase. In some embodiments, the DNA polymerase is a POLD1 DNA polymerase. In some embodiments, the DNA polymerase is a POLD2 DNA polymerase. In some embodiments, the DNA polymerase is a human POLD1 DNA polymerase. In some embodiments, the DNA polymerase is a human POLD2 DNA polymerase. In some embodiments, the DNA polymerase is a POLD3 DNA polymerase. In some embodiments, the DNA polymerase is a POLD4 DNA polymerase. In some embodiments, the DNA polymerase is a Pol-epsilon DNA polymerase. In some embodiments, the DNA polymerase is a POLE1 DNA polymerase. In some embodiments, the DNA polymerase is a POLE2 DNA polymerase. In some embodiments, the DNA polymerase is a POLE3 DNA polymerase. In some embodiments, the DNA polymerase is a Pol-eta (POLH) DNA polymerase. In some embodiments, the DNA polymerase is a Pol-iota (POLI) DNA polymerase. In some embodiments, the DNA polymerase is a Pol-kappa (POLK) DNA polymerase. In some embodiments, the DNA polymerase is a Rev1 DNA polymerase. In some embodiments, the DNA polymerase is a human Rev1 DNA polymerase. In some embodiments, the DNA polymerase is a viral DNA-dependent DNA polymerase. In some embodiments, the DNA polymerase is a B family DNA polymerases. In some embodiments, the DNA polymerase is a herpes simplex virus (HSV) UL30 DNA polymerase. In some embodiments, the DNA polymerase is a cytomegalovirus (CMV) UL54 DNA polymerase.

In some embodiments, the DNA polymerase is an archaeal polymerase. In some embodiments, the DNA polymerase is a Family B/pol I type DNA polymerase. For example, in some embodiments, the DNA polymerase is a homolog of Pfu from Pyrococcus furiosus. In some embodiments, the DNA polymerase is a pol II type DNA polymerase. For example, in some embodiments, the DNA polymerase is a homolog of P. furiosus DP1/DP2 2-subunit polymerase. In some embodiments, the DNA polymerase lacks 5′ to 3′ nuclease activity. Suitable DNA polymerases (pol I or pol II) can be derived from archaea with optimal growth temperatures that are similar to the desired assay temperatures.

In some embodiments, the DNA polymerase comprises a thermostable archaeal DNA polymerase. In some embodiments, the thermostable DNA polymerase is isolated or derived from Pyrococcus species (furiosus, species GB-D, woesii, abysii, horikoshii), Thermococcus species (kodakaraensis KOD1, litoralis, species 9 degrees North-7, species JDF-3, gorgonarius), Pyrodictium occultum, and Archaeoglobus fulgidus.

Polymerases may also be from eubacterial species. In some embodiments, the DNA polymerase is a Pol I family DNA polymerase. In some embodiments, the DNA polymerase is an E. coli Pol I DNA polymerase. In some embodiments, the DNA polymerase is a Pol II family DNA polymerase. In some embodiments, the DNA polymerase is a Pyrococcus furiosus (Pfu) Pol II DNA polymerase. In some embodiments, the DNA Polymerase is a Pol III family DNA polymerase. In some embodiments, the DNA Polymerase is a Pol IV family DNA polymerase. In some embodiments, the DNA polymerase is an E. coli Pol IV DNA polymerase. In some embodiments, the Pol I DNA polymerase is a DNA polymerase functional variant that lacks or has reduced 5′ to 3′ exonuclease activity.

Suitable thermostable pol I DNA polymerases can be isolated from a variety of thermophilic eubacteria, including Thermus species and Thermotoga maritima such as Thermus aquaticus (Taq), Thermus thermophilus (Tth) and Thermotoga maritima (Tma UlTma).

In some embodiments, a prime editor comprises an RNA-dependent DNA polymerase domain, for example, a reverse transcriptase (RT). A RT or an RT domain may be a wild type RT domain, a full-length RT domain, or may be a functional mutant, a functional variant, or a functional fragment thereof. An RT or an RT domain of a prime editor may comprise a wild-type RT, or may be engineered or evolved to contain specific amino acid substitutions, truncations, or variants. An engineered RT may comprise sequences or amino acid changes different from a naturally occurring RT. In some embodiments, the engineered RT may have improved reverse transcription activity over a naturally occurring RT or RT domain. In some embodiments, the engineered RT may have improved features over a naturally occurring RT, for example, improved thermostability, reverse transcription efficiency, or target fidelity. In some embodiments, a prime editor comprising the engineered RT has improved prime editing efficiency over a prime editor having a reference naturally occurring RT.

In some embodiments, a prime editor comprises a virus RT, for example, a retrovirus RT. Non-limiting examples of virus RT include Moloney murine leukemia virus (M-MLV or MLVRT); human T-cell leukemia virus type 1 (HTLV-1) RT; bovine leukemia virus (BLV) RT; Rous Sarcoma Virus (RSV) RT; human immunodeficiency virus (HIV) RT, M-MFV RT, Avian Sarcoma-Leukosis Virus (ASLV) RT, Rous Sarcoma Virus (RSV) RT, Avian Myeloblastosis Virus (AMV) RT, Avian Erythroblastosis Virus (AEV) Helper Virus MCAV RT, Avian Myelocytomatosis Virus MC29 Helper Virus MCAV RT, Avian Reticuloendotheliosis Virus (REV-T) Helper Virus REV-A RT, Avian Sarcoma Virus UR2 Helper Virus (UR2AV) RT, Avian Sarcoma Virus Y73 Helper Virus YAV RT, Rous Associated Virus (RAV) RT, and Myeloblastosis Associated Virus (MAV) RT, all of which may be suitably used in the methods and composition described herein.

In some embodiments, the prime editor comprises a wild type M-MLV RT. An exemplary sequence of a reference M-MLV RT is provided in SEQ ID NO: 3578.

In some embodiments, the prime editor comprises a M-MLV RT comprising one or more of amino acid substitutions P51X, S67X, E69X, L139X, T197X, D200X, H204X, F209X, E302X, T306X, F309X, W313X, T330X, L345X, L435X, N454X, D524X, E562X, D583X, H594X, L603X, E607X, or D653X as compared to the reference M-MLV RT as set forth in SEQ ID NO: 3578, where X is any amino acid other than the wild type amino acid. In some embodiments, the prime editor comprises a M-MLV RT comprising one or more of amino acid substitutions P51L, S67K, E69K, L139P, T197A, D200N, H204R, F209N, E302K, E302R, T306K, F309N, W313F, T330P, L345G, L435G, N454K, D524G, E562Q, D583N, H594Q, L603W, E607K, and D653N as compared to the reference M-MLV RT as set forth in SEQ ID NO: 3578. In some embodiments, the prime editor comprises a M-MLV RT comprising one or more amino acid substitutions D200N, T330P, L603W, T306K, and W313F as compared to the reference M-MLV RT as set forth in SEQ ID NO: 3578. In some embodiments, the prime editor comprises a M-MLV RT comprising amino acid substitutions D200N, T330P, L603W, T306K, and W313F as compared to the reference M-MLV RT as set forth in SEQ ID NO: 3578. In some embodiments, a prime editor comprises a M-MLV RT variant having the sequence as set forth in SEQ ID NO: 3579.

In some embodiments, an RT variant may be a functional fragment of a reference RT that has 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or up to 100, or up to 200, or up to 300, or up to 400, or up to 500 or more amino acid changes compared to a reference RT, e.g., a wild type RT or a reference RT. In some embodiments, the RT variant comprises a fragment of a reference RT, e.g., a wild type RT or a reference RT, such that the fragment is about 70% identical, about 80% identical, about 90% identical, about 95% identical, about 96% identical, about 97% identical, about 98% identical, about 99% identical, about 99.5% identical, or about 99.9% identical to the corresponding fragment of the reference RT. In some embodiments, the fragment is 30%, 35%, 40%, 45%, 50%, 55%, 60%, 65%, 70%, 75%, 80%, 85%, 90%, 95%, 96%, 97%, 98%, 99%, or 99.5% of the amino acid length of a corresponding reference RT (M-MLV reverse transcriptase) (e.g., SEQ ID NO: 3578).

In some embodiments, the RT functional fragment is at least 100 amino acids in length. In some embodiments, the fragment is at least 100, 150, 200, 250, 300, 350, 400, 450, 500, 550, or up to 600 or more amino acids in length.

In still other embodiments, the functional RT variant is truncated at the N-terminus or the C-terminus, or both, by a certain number of amino acids which results in a truncated variant which still retains sufficient DNA polymerase function. In some embodiments, the RT truncated variant has a truncation of at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 30, 40, 50, 60, 70, 80, 90, 100, 110, 120, 130, 140, 150, 160, 170, 180, 190, 200, 210, 220, 230, 240, or 250 amino acids at the N-terminal end compared to a reference RT. In some embodiments, the reference RT is has the sequence as set forth in SEQ ID NO: 3578. In other embodiments, the RT truncated variant has a truncation of at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 30, 40, 50, 60, 70, 80, 90, 100, 110, 120, 130, 140, 150, 160, 170, 180, 190, 200, 210, 220, 230, 240, or 250 amino acids at the C-terminal end compared to a reference RT. In some embodiments, the reference RT is has the sequence as set forth in SEQ ID NO: 3578. In still other embodiments, the RT truncated variant has a truncation at the N-terminal and the C-terminal end compared to a reference RT. In some embodiments, the reference RT is has the sequence as set forth in SEQ ID NO: 3578. In some embodiments, the N-terminal truncation and the C-terminal truncation are of the same length. In some embodiments, the N-terminal truncation and the C-terminal truncation are of different lengths.

For example, the prime editors disclosed herein may include a functional variant of a wild type M-MLV reverse transcriptase. In some embodiments, the prime editor comprises a functional variant of a reference M-MLV RT, wherein the functional variant of M-MLV RT is truncated after amino acid position 502 compared to a reference M-MLV RT as set forth in SEQ ID NO: 3578. In some embodiments, the functional variant of M-MLV RT further comprises a D200X, T306X, W313X, and/or T330X amino acid substitution compared to a reference M-MLV RT as set forth in SEQ ID NO: 3578, wherein X is any amino acid other than the original amino acid. In some embodiments, the functional variant of M-MLV RT further comprises a D200N, T306K, W313F, and/or T330P amino acid substitution compared to a reference M-MLV RT as set forth in SEQ ID NO: 3578, wherein X is any amino acid other than the original amino acid. A DNA sequence encoding a prime editor comprising this truncated RT is 522 bp smaller than the DNA encoding the reference M-MLV RT (e.g. the RT as set forth in SEQ ID NO: 3578), and therefore makes it potentially useful for applications where delivery of the DNA sequence is challenging due to its size (i.e., adeno-associated virus and lentivirus delivery).

In some embodiments, a prime editor comprises a eukaryotic RT, for example, a yeast, drosophila, rodent, or primate RT. In some embodiments, the prime editor comprises a Group II intron RT, for example, a. Geobacillus stearothermophilus Group II Intron (GsI-IIC) RT or a Eubacterium rectale group II intron (Eu.re.I2) RT. In some embodiments, the prime editor comprises a retron RT.

### Programmable DNA Binding Domain

In some embodiments, the DNA-binding domain of a prime editor is a programmable DNA binding domain. A programmable DNA binding domain refers to a protein domain that is designed to bind a specific nucleic acid sequence, e.g., a target DNA or a target RNA. In some embodiments, the DNA-binding domain is a polynucleotide programmable DNA-binding domain that can associate with a guide polynucleotide (e.g., a PEgRNA) that guides the DNA-binding domain to a specific DNA sequence, e.g., a search target sequence in a target gene. In some embodiments, the DNA-binding domain comprises a Clustered Regularly Interspaced Short Palindromic Repeats (CRISPR) Associated (Cas) protein. A Cas protein may comprise any Cas protein described herein or a functional fragment or functional variant thereof. In some embodiments, a DNA-binding domain may also comprise a zinc-finger protein domain. In other cases, a DNA-binding domain comprises a transcription activator-like effector domain (TALE). In some embodiments, the DNA-binding domain comprises a DNA nuclease. For example, the DNA-binding domain of a prime editor may comprise an RNA-guided DNA endonuclease, e.g., a Cas protein. In some embodiments, the DNA-binding domain comprises a zinc finger nuclease (ZFN) or a transcription activator like effector domain nuclease (TALEN), where one or more zinc finger motifs or TALE motifs are associated with one or more nucleases, e.g., a Fok I nuclease domain.

In some embodiments, the DNA-binding domain comprise a nuclease activity. In some embodiments, the DNA-binding domain of a prime editor comprises an endonuclease domain having single strand DNA cleavage activity. For example, the endonuclease domain may comprise a FokI nuclease domain. In some embodiments, the DNA-binding domain of a prime editor comprises a nuclease having full nuclease activity. In some embodiments, the DNA-binding domain of a prime editor comprises a nuclease having modified or reduced nuclease activity as compared to a wild type endonuclease domain. For example, the endonuclease domain may comprise one or more amino acid substitutions as compared to a wild type endonuclease domain. In some embodiments, the DNA-binding domain of a prime editor has nickase activity. In some embodiments, the DNA-binding domain of a prime editor comprises a Cas protein domain that is a nickase. In some embodiments, compared to a wild type Cas protein, the Cas nickase comprises one or more amino acid substitutions in a nuclease domain that reduces or abolishes its double strand nuclease activity but retains DNA binding activity. In some embodiments, the Cas nickase comprises an amino acid substitution in a HNH domain. In some embodiments, the Cas nickase comprises an amino acid substitution in a RuvC domain.

In some embodiments, the DNA-binding domain comprises a CRISPR associated protein (Cas protein) domain. A Cas protein may be a Class 1 or a Class 2 Cas protein. A Cas protein can be a type I, type II, type III, type IV, type V Cas protein, or a type VI Cas protein. Non-limiting examples of Cas proteins include Cas9, Cas12a (Cpf1), Cas12e (CasX), Cas12d (CasY), Cas12b1 (C2c1), Cas12b2, Cas12c (C2c3), C2c4, C2c8, C2c5, C2c10, C2c9, Cas14a, Cas14b, Cas14c, Cas14d, Cas14e, Cas14f, Cas14g, Cas14h, Cas14u, Cns2, Cas Φ, and homologs, functional fragments, or modified versions thereof. A Cas protein can be a chimeric Cas protein that is fused to other proteins or polypeptides. A Cas protein can be a chimera of various Cas proteins, for example, comprising domains of Cas proteins from different organisms.

A Cas protein, e.g., Cas9, can be from any suitable organism. In some aspects, the organism is Streptococcus pyogenes (S. pyogenes). In some aspects, the organism is Staphylococcus aureus (S. aureus). In some aspects, the organism is Streptococcus thermophilus (S. thermophilus). In some embodiments, the organism is Staphylococcus lugdunensis.

A Cas protein, e.g., Cas9, can be a wild type or a modified form of a Cas protein. A Cas protein, e.g., Cas9, can be a nuclease active variant, nuclease inactive variant, a nickase, or a functional variant or functional fragment of a wild type Cas protein. A Cas protein, e.g., Cas9, can comprise an amino acid change such as a deletion, insertion, substitution, fusion, chimera, or any combination thereof relative to a wild-type version of the Cas protein. A Cas protein can be a polypeptide with at least about 50%, 60%, 70%, 80%, 90%, 91%, 92%, 93%, 94%, 95%, 96%, 97%, 98%, 99%, or 100% sequence identity or sequence similarity to a wild type exemplary Cas protein.

A Cas protein, e.g., Cas9, may comprise one or more domains. Non-limiting examples of Cas domains include, guide nucleic acid recognition and/or binding domain, nuclease domains (e.g., DNase or RNase domains, RuvC, HNH), DNA binding domain, RNA binding domain, helicase domains, protein-protein interaction domains, and dimerization domains. In various embodiments, a Cas protein comprises a guide nucleic acid recognition and/or binding domain that can interact with a guide nucleic acid, and one or more nuclease domains that comprise catalytic activity for nucleic acid cleavage.

In some embodiments, a Cas protein, e.g., Cas9, comprises one or more nuclease domains. A Cas protein can comprise an amino acid sequence having at least about 50%, 60%, 70%, 80%, 90%, 91%, 92%, 93%, 94%, 95%, 96%, 97%, 98%, 99%, or 100% sequence identity to a nuclease domain (e.g., RuvC domain, HNH domain) of a wild-type Cas protein. In some embodiments, a Cas protein comprises a single nuclease domain. For example, a Cpf1 may comprise a RuvC domain but lacks HNH domain. In some embodiments, a Cas protein comprises two nuclease domains, e.g., a Cas9 protein can comprise an HNH nuclease domain and a RuvC nuclease domain.

In some embodiments, a prime editor comprises a Cas protein, e.g., Cas9, wherein all nuclease domains of the Cas protein are active. In some embodiments, a prime editor comprises a Cas protein having one or more inactive nuclease domains. One or a plurality of the nuclease domains (e.g., RuvC, HNH) of a Cas protein can be deleted or mutated so that they are no longer functional or comprise reduced nuclease activity. In some embodiments, a Cas protein, e.g., Cas9, comprising mutations in a nuclease domain has reduced (e.g., nickase) or abolished nuclease activity while maintaining its ability to target a nucleic acid locus at a search target sequence when complexed with a guide nucleic acid, e.g., a PEgRNA.

In some embodiments, a prime editor comprises a Cas nickase that can bind to the target gene in a sequence-specific manner and generate a single-strand break at a protospacer within double-stranded DNA in the target gene, but not a double-strand break. For example, the Cas nickase can cleave the edit strand (i.e., the PAM strand) or the non-edit strand of the target gene, but may not cleave both. In some embodiments, a prime editor comprises a Cas nickase comprising two nuclease domains (e.g., Cas9), with one of the two nuclease domains modified to lack catalytic activity or deleted. In some embodiments, the Cas nickase of a prime editor comprises a nuclease inactive RuvC domain and a nuclease active HNH domain. In some embodiments, the Cas nickase of a prime editor comprises a nuclease inactive HNH domain and a nuclease active RuvC domain. In some embodiments, a prime editor comprises a Cas9 nickase having an amino acid substitution in the RuvC domain. In some embodiments, the Cas9 nickase comprises a DIOX amino acid substitution compared to a wild type S. pyogenes Cas9 as set forth in SEQ ID NO: 3580, wherein X is any amino acid other than D. In some embodiments, a prime editor comprises a Cas9 nickase having an amino acid substitution in the HNH domain. In some embodiments, the Cas9 nickase comprises a H840X amino acid substitution compared to a wild type S. pyogenes Cas9 as set forth in SEQ ID NO: 3580, wherein X is any amino acid other than H.

In some embodiments, a prime editor comprises a Cas protein that can bind to the target gene in a sequence-specific manner but lacks or has abolished nuclease activity and may not cleave either strand of a double stranded DNA in a target gene. Abolished activity or lacking activity can refer to an enzymatic activity less than 1%, less than 2%, less than 3%, less than 4%, less than 5%, less than 6%, less than 7%, less than 8%, less than 9%, or less than 10% activity compared to a wild-type exemplary activity (e.g., wild-type Cas9 nuclease activity). In some embodiments, a Cas protein of a prime editor completely lacks nuclease activity. A nuclease, e.g., Cas9, that lacks nuclease activity may be referred to as nuclease inactive or “nuclease dead” (abbreviated by “d”). A nuclease dead Cas protein (e.g., dCas, dCas9) can bind to a target polynucleotide but may not cleave the target polynucleotide. In some aspects, a dead Cas protein is a dead Cas9 protein. In some embodiments, a prime editor comprises a nuclease dead Cas protein wherein all of the nuclease domains (e.g., both RuvC and HNH nuclease domains in a Cas9 protein; RuvC nuclease domain in a Cpf1 protein) are mutated to lack catalytic activity, or are deleted.

A Cas protein can be modified. A Cas protein, e.g., Cas9, can be modified to increase or decrease nucleic acid binding affinity, nucleic acid binding specificity, and/or enzymatic activity. Cas proteins can also be modified to change any other activity or property of the protein, such as stability. For example, one or more nuclease domains of the Cas protein can be modified, deleted, or inactivated, or a Cas protein can be truncated to remove domains that are not essential for the function of the protein or to optimize (e.g., enhance or reduce) the activity of the Cas protein.

A Cas protein can be a fusion protein. For example, a Cas protein can be fused to a cleavage domain, an epigenetic modification domain, a transcriptional regulation domain, or a polymerase domain. A Cas protein can also be fused to a heterologous polypeptide providing increased or decreased stability. The fused domain or heterologous polypeptide can be located at the N-terminus, the C-terminus, or internally within the Cas protein.

In some embodiments, the Cas protein of a prime editor is a Class 2 Cas protein. In some embodiments, the Cas protein is a type II Cas protein. In some embodiments, the Cas protein is a Cas9 protein, a modified version of a Cas9 protein, a Cas9 protein homolog, mutant, variant, or a functional fragment thereof. As used herein, a Cas9, Cas9 protein, Cas9 polypeptide or a Cas9 nuclease refers to an RNA guided nuclease comprising one or more Cas9 nuclease domains and a Cas9 gRNA binding domain having the ability to bind a guide polynucleotide, e.g., a PEgRNA. A Cas9 protein may refer to a wild type Cas9 protein from any organism or a homolog, ortholog, or paralog from any organisms; any functional mutants or functional variants thereof; or any functional fragments or domains thereof. In some embodiments, a prime editor comprises a full-length Cas9 protein. In some embodiments, the Cas9 protein can generally comprises at least about 50%, 60%, 70%, 80%, 90%, 100% sequence identity to a wild type reference Cas9 protein (e.g., Cas9 from S. pyogenes). In some embodiments, the Cas9 comprises an amino acid change such as a deletion, insertion, substitution, fusion, chimera, or any combination thereof as compared to a wild type reference Cas9 protein.

In some embodiments, a Cas9 protein may comprise a Cas9 protein from Streptococcus pyogenes (Sp), Staphylococcus aureus (Sa), Streptococcus canis (Sc), Streptococcus thermophilus (St), Staphylococcus lugdunensis (Slu), Neisseria meningitidis (Nm), Campylobacter jejuni (Cj), Francisella novicida (Fn), or Treponema denticola (Td), or any Cas9 homolog or ortholog from an organism known in the art. In some embodiments, a Cas9 polypeptide is a SpCas9 polypeptide. In some embodiments, a Cas9 polypeptide is a SaCas9 polypeptide. In some embodiments, a Cas9 polypeptide is a ScCas9 polypeptide. In some embodiments, a Cas9 polypeptide is a StCas9 polypeptide. In some embodiments, a Cas9 polypeptide is a SluCas9 polypeptide. In some embodiments, a Cas9 polypeptide is a NmCas9 polypeptide. In some embodiments, a Cas9 polypeptide is a CjCas9 polypeptide. In some embodiments, a Cas9 polypeptide is a FnCas9 polypeptide. In some embodiments, a Cas9 polypeptide is a TdCas9 polypeptide. In some embodiments, a Cas9 polypeptide is a chimera comprising domains from two or more of the organisms described herein or those known in the art. In some embodiments, a Cas9 polypeptide is a Cas9 polypeptide from Streptococcus macacae. In some embodiments, a Cas9 polypeptide is a Cas9 polypeptide generated by replacing a PAM interaction domain of a SpCas9 with that of a Streptococcus macacae Cas9 (Spy-mac Cas9).

In some embodiments, a Cas9 is a chimeric Cas9, e.g., modified Cas9; e.g., synthetic RNA-guided nucleases (sRGNs), e.g., modified by DNA family shuffling, e.g., sRGN3.1, sRGN3.3. In some embodiments, the DNA family shuffling comprises, fragmentation and reassembly of parental Cas9 genes, e.g., one or more of Cas9s from Staphylococcus hyicus (Shy), Staphylococcus lugdunensis (Slu), Staphylococcus microti (Smi), and Staphylococcus pasteuri (Spa).

An exemplary wild type Streptococcus pyogenes Cas9 (SpCas9) amino acid sequence is provided in SEQ ID NO: 3580.

In some embodiments, a prime editor comprises a Cas9 protein from Staphylococcus lugdunensis (Slu Cas9). An exemplary amino acid sequence of a wild type Slu Cas9 is provided in SEQ ID NO: 3583.

In some embodiments, a Cas9 protein comprises a variant Cas9 protein containing one or more amino acid substitutions. In some embodiments, a wild type Cas9 protein comprises a RuvC domain and an HNH domain. In some embodiments, a prime editor comprises a nuclease active Cas9 protein that may cleave both strands of a double stranded target DNA sequence. In some embodiments, the nuclease active Cas9 protein comprises a functional RuvC domain and a functional HNH domain. In some embodiments, a prime editor comprises a Cas9 nickase that can bind to a guide polynucleotide and recognize a target DNA, but can cleave only one strand of a double stranded target DNA. In some embodiments, the Cas9 nickase comprises only one functional RuvC domain or one functional HNH domain. In some embodiments, a prime editor comprises a Cas9 that has a non-functional HNH domain and a functional RuvC domain. In some embodiments, the prime editor can cleave the edit strand (i.e., the PAM strand), but not the non-edit strand of a double stranded target DNA sequence. In some embodiments, a prime editor comprises a Cas9 having a non-functional RuvC domain that can cleave the target strand (i.e., the non-PAM strand), but not the edit strand of a double stranded target DNA sequence. In some embodiments, a prime editor comprises a Cas9 that has neither a functional RuvC domain nor a functional HNH domain, which may not cleave any strand of a double stranded target DNA sequence.

In some embodiments, a prime editor comprises a Cas9 having a mutation in the RuvC domain that reduces or abolishes the nuclease activity of the RuvC domain. In some embodiments, the Cas9 comprise a mutation at amino acid D10 as compared to a wild type SpCas9 as set forth in SEQ ID NO: 3580, or a corresponding mutation thereof. In some embodiments, the Cas9 comprise a D10A mutation as compared to a wild type SpCas9 as set forth in SEQ ID NO: 3580, or a corresponding mutation thereof. In some embodiments, the Cas9 polypeptide comprise a mutation at amino acid D10, G12, and/or G17 as compared to a wild type SpCas9 as set forth in SEQ ID NO: 3580, or a corresponding mutation thereof. In some embodiments, the Cas9 polypeptide comprise a D10A mutation, a G12A mutation, and/or a G17A mutation as compared to a wild type SpCas9 as set forth in SEQ ID NO: 3580, or a corresponding mutation thereof.

In some embodiments, a prime editor comprises a Cas9 polypeptide having a mutation in the HNH domain that reduces or abolishes the nuclease activity of the HNH domain. In some embodiments, the Cas9 polypeptide comprise a mutation at amino acid H840 as compared to a wild type SpCas9 as set forth in SEQ ID NO: 3580, or a corresponding mutation thereof. In some embodiments, the Cas9 polypeptide comprise a H840A mutation as compared to a wild type SpCas9 as set forth in SEQ ID NO: 3580, or a corresponding mutation thereof. In some embodiments, the Cas9 polypeptide comprises a mutation at amino acid E762, D839, H840, N854, N856, N863, H982, H983, A984, D986, and/or a A987 as compared to a wild type SpCas9 as set forth in SEQ ID NO: 3580, or a corresponding mutation thereof. In some embodiments, the Cas9 polypeptide comprise a E762A, D839A, H840A, N854A, N856A, N863A, H982A, H983A, A984A, and/or a D986A mutation as compared to a wild type SpCas9 as set forth in SEQ ID NO: 3580, or a corresponding mutation thereof.

In some embodiments, a prime editor comprises a Cas9 having one or more amino acid substitutions in both the HNH domain and the RuvC domain that reduce or abolish the nuclease activity of both the HNH domain and the RuvC domain. In some embodiments, the prime editor comprises a nuclease inactive Cas9, or a nuclease dead Cas9 (dCas9). In some embodiments, the dCas9 comprises a H840X substitution and a DIOX mutation compared to a wild type SpCas9 as set forth in SEQ ID NO: 3580 or corresponding mutations thereof, wherein X is any amino acid other than H for the H840X substitution and any amino acid other than D for the DIOX substitution. In some embodiments, the dead Cas9 comprises a H840A and a D10A mutation as compared to a wild type SpCas9 as set forth in SEQ ID NO: 3580, or corresponding mutations thereof.

In some embodiments, the N-terminal methionine is removed from a Cas9 nickase, or from any Cas9 variant, ortholog, or equivalent disclosed or contemplated herein. For example, methionine-minus Cas9 nickases, or a variant thereof having an amino acid sequence that has at least 80%, at least 85%, at least 90%, at least 95%, or at least 99% sequence identity thereto.

In some embodiments, a prime editor comprises a Streptococcus pyogenes Cas9 (SpCas9) having a nuclease inactivating mutation in the HNH domain (a SpCas9 nickase). In some embodiments, the SpCas9 nickase lacks the N-terminus methionine relative to a corresponding reference SpCas9 (e.g., wild type SpCas9). In some embodiments, a prime editor comprises a SpCas9 nickase having the sequences as provide in SEQ ID NO: 3581 (SpCas9 H840A nickase including the N-terminal methionine). In some embodiments, a prime editor comprises a SpCas9 nickase having the sequences as provide in SEQ ID NO: 3582 (SpCas9 H840A nickase lacking the N-terminal methionine).

In some embodiments, the SpCas9 nickase further comprises a R221K and/or a N394K amino acid substitution compared to a reference SpCas9 sequence set forth in SEQ ID NO: 3580. In some embodiments, the SpCas9 nickase comprises a sequence as set forth in SEQ ID NO: 3623.

In some embodiments, a prime editor comprises a Staphylococcus lugdunensis (SluCas9) having a nuclease inactivating mutation in the HNH domain (a SluCas9 nickase). In some embodiments, the SluCas9 nickase lacks the N-terminus methionine relative to a corresponding reference SluCas9 (e.g., wild type SluCas9). In some embodiments, a prime editor comprises a SluCas9 nickase having the sequences as provide in SEQ ID NO: 3584 (SluCas9 H840A nickase including the N-terminal methionine). In some embodiments, a prime editor comprises a SluCas9 nickase having the sequences as provide in SEQ ID NO: 3585 (SluCas9 H840A nickase lacking the N-terminal methionine).

Besides dead Cas9 and Cas9 nickase variants, the Cas9 proteins used herein may also include other Cas9 variants having at least about 70% identity, at least about 80% identity, at least about 90% identity, at least about 95% identity, at least about 96% identity, at least about 97% identity, at least about 98% identity, at least about 99% identity, at least about 99.5% identity, or at least about 99.9% identity to any reference Cas9 protein, including any wild type Cas9, or mutant Cas9 (e.g., a dead Cas9 or Cas9 nickase), or fragment Cas9, or circular permutant Cas9, or other variant of Cas9 disclosed herein or known in the art. In some embodiments, a Cas9 variant may have 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50 or more amino acid changes compared to a reference Cas9, e.g., a wild type Cas9. In some embodiments, the Cas9 variant comprises a fragment of a reference Cas9 (e.g., a gRNA binding domain or a DNA-cleavage domain), such that the fragment is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to the corresponding fragment of the reference Cas9, e.g., a wild type Cas9. In some embodiments, the fragment is at least 30%, at least 35%, at least 40%, at least 45%, at least 50%, at least 55%, at least 60%, at least 65%, at least 70%, at least 75%, at least 80%, at least 85%, at least 90%, at least 95% identical, at least 96%, at least 97%, at least 98%, at least 99%, or at least 99.5% of the amino acid length of a corresponding wild type Cas9.

In some embodiments, a Cas9 fragment is a functional fragment that retains one or more Cas9 activities. In some embodiments, the Cas9 fragment is at least 100 amino acids in length. In some embodiments, the fragment is at least 100, 150, 200, 250, 300, 350, 400, 450, 500, 550, 600, 650, 700, 750, 800, 850, 900, 950, 1000, 1050, 1100, 1150, 1200, 1250, or at least 1300 amino acids in length.

In some embodiments, a prime editor comprises a Cas protein, e.g., Cas9, containing modifications that allow altered PAM recognition. In prime editing using a Cas-protein-based prime editor, a “protospacer adjacent motif (PAM)”, PAM sequence, or PAM-like motif, may be used to refer to a short DNA sequence immediately following the protospacer on the PAM strand of the target gene. In some embodiments, the PAM is recognized by the Cas nuclease in the prime editor during prime editing. In certain embodiments, the PAM is required for target binding of the Cas protein. The specific PAM sequence required for Cas protein recognition may depend on the specific type of the Cas protein. A PAM can be 1, 2, 3, 4, 5, 6, 7, 8, 9, 10 or more nucleotides in length. In some embodiments, a PAM is between 2-6 nucleotides in length. In some embodiments, the PAM can be a 5′ PAM (i.e., located upstream of the 5′ end of the protospacer). In other embodiments, the PAM can be a 3′ PAM (i.e., located downstream of the 5′ end of the protospacer). In some embodiments, the Cas protein of a prime editor recognizes a canonical PAM, for example, a SpCas9 recognizes 5′-NGG-3′ PAM. In some embodiments, the Cas protein of a prime editor has altered or non-canonical PAM specificities. Exemplary PAM sequences and corresponding Cas variants are described in Table 43 below. It should be appreciated that for each of the variants provided, the Cas protein comprises one or more of the amino acid substitutions as indicated compared to a wild type Cas protein sequence, for example, the SpCas9 as set forth in SEQ ID NO: 3580. The PAM motifs as shown in Table 43 below are in the order of 5′ to 3′. The nucleotides listed in Table 43 are represented by the base codes as provided in the Handbook on Industrial Property Information and Documentation, World Intellectual Property Organization (WIPO) Standard ST.26, Version 1.4. For example, an “R” in Table 43 represents the nucleotide A or G; “W” in Table 43 represents A or T; and “V” in Table 43 represents A or C or G.

TABLE 43
Cas protein variants and corresponding PAM sequences

| Variant | PAM |
|---|---|
| spCas9 (wild type) | NGG, NGA, NAG |
| spCas9-VRVRFRR (also referred to as SpCas9-NG) | NG |
| R1335V/L1111R/D1135V/G1218R/E1219F/A1322R/T1337R | |
| spCas9-VQR (D1135V/R1335Q/T1337R) | NGA |
| spCas9-EQR (D1135E/R1335Q/T1337R) | NGA |
| spCas9-VRER (D1135V/G1218R/R1335E/T1337R) | NGCG |
| xCas9 (E480K, E543D, E1219V, K294R, Q1256K, A262T, | NGN |
| S409I, M694I) | |
| SluCa9 | NNGG |
| saCas9 | NNGRRT, NNGRRN |
| saCas9-KKH (E782K, N968K, R1015H) | NNNRRT |
| spCas9-MQKSER (D1135M, S1136Q, G1218K, E1219S, | NGCG/NGCN |
| R1335E, T1337R) | |
| spCas9-LRKIQK (D1135L, S1136R, G1218K, E1219I, | NGTN |
| R1335Q, T1337K) | |
| spCas9-LRVSQK (D1135L, S1136R, G1218V, E1219S, | NGTN |
| R1335Q, T1337K) | |
| spCas9-LRVSQL(D1135L, S1136R, G1218V, E1219S, | NGTN |
| R1335Q, T1337L) | |
| Cpf1 | TTTV |
| Spy-Mac | NAA |
| NmCas9 | NNNNGATT |
| StCas9 | NNAGAAW |
| TdCas9 | NAAAAC |
| spCas9-VRQR (D1135V, G1218R, R1335Q, T1337R) | NGA |
| SpG Cas9 (D1135L, S1136W, G1218K, E1219Q, R1335Q, | NGN |
| T1337R) | |
| SyRY Cas9 | NRN |
| (A61R, L1111R, N1317R, A1322R, and R1333P) | |
| sRGN1, sRGN2, sRGN4, sRGN3.1, sRGN3.3 | NNGG |

Exemplary Cas9s that allow alternative PAM recognition are provided in SEQ ID NO: 3590 (SpCas9-NG), SEQ ID NO: 3591 (SpCas9-NG H840A nickase), SEQ ID NO: 3592 (SpCas9-NG H839A nickase lacking N-terminal methionine), SEQ ID NO: 3593 (SpCas9-VRQR), SEQ ID NO: 3594 (SpCas9-VRQR H840A nickase), SEQ ID NO: 3595 (SpCas9-VRQR H839A nickase lacking N-terminal methionine), SEQ ID NO: 3596 (SpRY Cas9), SEQ ID NO: 3697 (SpRY Cas9 H840A nickase), SEQ ID NO: 3598 (SpRY Cas9 H839A nickase lacking N-terminal methionine), SEQ ID NO: 3599 (sRGN3.1), SEQ ID NO: 3600 (sRGN3.1 N585A nickase), SEQ ID NO: 3601 (sRNA3.1 N584A nickase lacking N-terminal methionine), SEQ ID NO: 3602 (sRGN3.3), SEQ ID NO: 3603 (sRGN3.3 N585A nickase), SEQ ID NO: 3604 (sRGN3.3 N584A nickase lacking N-terminal methionine), SEQ ID NO: 3605 (SpG Cas9), SEQ ID NO: 3606 (SpG Cas9 H840A nikcase), and SEQ ID NO: 3607 (SpG Cas9 H839A nickase lacking N-terminal methionine). In some embodiments, a prime editor comprises a Cas9 polypeptide comprising one or more mutations selected from the group consisting of: A61R, L111R, D1135V, R221K, A262T, R324L, N394K, S409I, S409I, E427G, E480K, M495V, N497A, Y515N, K526E, F539S, E543D, R654L, R661A, R661L, R691A, N692A, M694A, M694I, Q695A, H698A, R753G, M763I, K848A, K890N, Q926A, K1003A, R1060A, L1111R, R1114G, D1135E, D1135L, D1135N, S1136W, V1139A, D1180G, G1218K, G1218R, G1218S, E1219Q, E1219V, E1219V, Q1221H, P1249S, E1253K, N1317R, A1320V, P1321S, A1322R, I1322V, D1332G, R1332N, A1332R, R1333K, R1333P, R1335L, R1335Q, R1335V, T1337N, T1337R, S1338T, H1349R, and any combinations thereof as compared to a wild type SpCas9 polypeptide as set forth in SEQ ID NO: 3580.

In some embodiments, a prime editor comprises a SaCas9 polypeptide. In some embodiments, the SaCas9 polypeptide comprises one or more of mutations E782K, N968K, and R1015H as compared to a wild type SaCas9. In some embodiments, a prime editor comprises a FnCas9 polypeptide, for example, a wild type FnCas9 polypeptide or a FnCas9 polypeptide comprising one or more of mutations E1369R, E1449H, or R1556A as compared to the wild type FnCas9. In some embodiments, a prime editor comprises a Sc Cas9, for example, a wild type ScCas9 or a ScCas9 polypeptide comprises one or more of mutations I367K, G368D, I369K, H371L, T375S, T376G, and T1227K as compared to the wild type ScCas9. In some embodiments, a prime editor comprises a St1 Cas9 polypeptide, a St3 Cas9 polypeptide, or a Slu Cas9 polypeptide.

In some embodiments, a prime editor comprises a Cas polypeptide that comprises a circular permutant Cas variant. For example, a Cas9 polypeptide of a prime editor may be engineered such that the N-terminus and the C-terminus of a Cas9 protein (e.g., a wild type Cas9 protein, or a Cas9 nickase) are topically rearranged to retain the ability to bind DNA when complexed with a guide RNA (gRNA). An exemplary circular permutant configuration may be N-terminus-[original C-terminus]-[original N-terminus]-C-terminus. Any of the Cas9 proteins described herein, including any variant, ortholog, or naturally occurring Cas9 or equivalent thereof, may be reconfigured as a circular permutant variant.

In various embodiments, the circular permutants of a Cas protein, e.g., a Cas9, may have the following structure: N-terminus-[original C-terminus]-[optional linker]-[original N-terminus]-C-terminus. In some embodiments, a circular permutant Cas9 comprises any one of the following structures (amino acid positions as set forth in SEQ ID NO: 3580.
N-terminus-[1268-1368]-[optional linker]-[1-1267]-C-terminus;
N-terminus-[1168-1368]-[optional linker]-[1-1167]-C-terminus;
N-terminus-[1068-1368]-[optional linker]-[1-1067]-C-terminus;
N-terminus-[968-1368]-[optional linker]-[1-967]-C-terminus;
N-terminus-[868-1368]-[optional linker]-[1-867]-C-terminus;
N-terminus-[768-1368]-[optional linker]-[1-767]-C-terminus;
N-terminus-[668-1368]-[optional linker]-[1-667]-C-terminus;
N-terminus-[568-1368]-[optional linker]-[1-567]-C-terminus;
N-terminus-[468-1368]-[optional linker]-[1-467]-C-terminus;
N-terminus-[368-1368]-[optional linker]-[1-367]-C-terminus;
N-terminus-[268-1368]-[optional linker]-[1-267]-C-terminus;
N-terminus-[168-1368]-[optional linker]-[1-167]-C-terminus;
N-terminus-[68-1368]-[optional linker]-[1-67]-C-terminus;
N-terminus-[10-1368]-[optional linker]-[1-9]-C-terminus, or the corresponding circular permutants of other Cas9 proteins (including other Cas9 orthologs, variants, etc).

In some embodiments, a circular permutant Cas9 comprises any one of the following structures (amino acid positions as set forth in SEQ ID NO: 3580):
N-terminus-[102-1368]-[optional linker]-[1-101]-C-terminus;
N-terminus-[1028-1368]-[optional linker]-[1-1027]-C-terminus;
N-terminus-[1041-1368]-[optional linker]-[1-1043]-C-terminus;
N-terminus-[1249-1368]-[optional linker]-[1-1248]-C-terminus; or
N-terminus-[1300-1368]-[optional linker]-[1-1299]-C-terminus, or the corresponding circular permutants of other Cas9 proteins (including other Cas9 orthologs, variants, etc).

In some embodiments, a circular permutant Cas9 comprises any one of the following structures (amino acid positions as set forth in SEQ ID NO: 3580):
N-terminus-[103-1368]-[optional linker]-[1-102]-C-terminus:
N-terminus-[1029-1368]-[optional linker]-[1-1028]-C-terminus;
N-terminus-[1042-1368]-[optional linker]-[1-1041]-C-terminus;
N-terminus-[1250-1368]-[optional linker]-[1-1249]-C-terminus; or
N-terminus-[1301-1368]-[optional linker]-[1-1300]-C-terminus, or the corresponding circular permutants of other Cas9 proteins (including other Cas9 orthologs, variants, etc).

In some embodiments, the circular permutant can be formed by linking a C-terminal fragment of a Cas9 to an N-terminal fragment of a Cas9, either directly or by using a linker, such as an amino acid linker. In some embodiments, the C-terminal fragment may correspond to the C-terminal 95% or more of the amino acids of a Cas9 (e.g., amino acids about 1300-1368 as set forth in SEQ ID NO: 3580 or corresponding amino acid positions thereof), or the C-terminal 90%, 85%, 80%, 75%, 70%, 65%, 60%, 55%, 50%, 45%, 40%, 35%, 30%, 25%, 20%, 15%, 10%, or 5% or more of a Cas9. The N-terminal portion may correspond to the N-terminal 95% or more of the amino acids of a Cas9 (e.g., amino acids about 1-1300 as set forth in SEQ ID NO: 3580 or corresponding amino acid positions thereof), or the N-terminal 90%, 85%, 80%, 75%, 70%, 65%, 60%, 55%, 50%, 45%, 40%, 35%, 30%, 25%, 20%, 15%, 10%, or 5% or more of a Cas9 (e.g., as set forth in SEQ ID NO: 3580 or corresponding amino acid positions thereof).

In some embodiments, the circular permutant can be formed by linking a C-terminal fragment of a Cas9 to an N-terminal fragment of a Cas9, either directly or by using a linker, such as an amino acid linker. In some embodiments, the C-terminal fragment that is rearranged to the N-terminus includes or corresponds to the C-terminal 30% or less of the amino acids of a Cas9 (e.g., amino acids 1012-1368 as set forth in SEQ ID NO: 3580 or corresponding amino acid positions thereof). In some embodiments, the C-terminal fragment that is rearranged to the N-terminus, includes or corresponds to the C-terminal 30%, 29%, 28%, 27%, 26%, 25%, 24%, 23%, 22%, 21%, 20%, 19%, 18%, 17%, 16%, 15%, 14%, 3%, 12%, 11%, 10%, 9%, 8%, 7%, 6%, 5%, 4%, 3%, 2%, or 1% of the amino acids of a Cas9 (e.g., as set forth in SEQ ID NO: 3580 or corresponding amino acid positions thereof). In some embodiments, the C-terminal fragment that is rearranged to the N-terminus, includes or corresponds to the C-terminal 410 residues or less of a Cas9 (e.g., as set forth in SEQ ID NO: 3580 or corresponding amino acid positions thereof). In some embodiments, the C-terminal portion that is rearranged to the N-terminus, includes or corresponds to the C-terminal 410, 400, 390, 380, 370, 360, 350, 340, 330, 320, 310, 300, 290, 280, 270, 260, 250, 240, 230, 220, 210, 200, 190, 180, 170, 160, 150, 140, 130, 120, 110, 100, 90, 80, 70, 60, 50, 40, 30, 20, or 10 residues of a Cas9 (e.g., as set forth in SEQ ID NO: 3580 or corresponding amino acid positions thereof). In some embodiments, the C-terminal portion that is rearranged to the N-terminus includes or corresponds to the C-terminal 357, 341, 328, 120, or 69 residues of a Cas9 (e.g., as set forth in SEQ ID NO: 3580 or corresponding amino acid positions thereof).

In other embodiments, circular permutant Cas9 variants may be a topological rearrangement of a Cas9 primary structure based on the following method, which is based on S. pyogenes Cas9 of SEQ ID NO: 3580: (a) selecting a circular permutant (CP) site corresponding to an internal amino acid residue of the Cas9 primary structure, which dissects the original protein into two halves: an N-terminal region and a C-terminal region; (b) modifying the Cas9 protein sequence (e.g., by genetic engineering techniques) by moving the original C-terminal region (comprising the CP site amino acid) to precede the original N-terminal region, thereby forming a new N-terminus of the Cas9 protein that now begins with the CP site amino acid residue. The CP site can be located in any domain of the Cas9 protein, including, for example, the helical-II domain, the RuvCIII domain, or the CTD domain. For example, the CP site may be located (as set forth in SEQ ID NO: 3580 or corresponding amino acid positions thereof) at original amino acid residue 181, 199, 230, 270, 310, 1010, 1016, 1023, 1029, 1041, 1247, 1249, or 1282. Thus, once relocated to the N-terminus, original amino acid 181, 199, 230, 270, 310, 1010, 1016, 1023, 1029, 1041, 1247, 1249, or 1282 would become the new N-terminal amino acid. Nomenclature of these CP-Cas9 proteins may be referred to as Cas9-CP¹⁸¹, Cas9-CP¹⁹⁹, Cas9-CP²³⁰, Cas9-CP²⁷⁰, Cas9-CP³¹⁰, Cas9-CP¹⁰¹⁰, Cas9-CP¹⁰¹⁶, Cas9-CP¹⁰²³, Cas9-CP¹⁰²⁹, Cas9-CP¹⁰⁴¹, Cas9-CP¹²⁴⁷, Cas9-CP¹²⁴⁹, and Cas9-CP¹²⁸², respectively. This description is not meant to be limited to making CP variants from SEQ ID NO: 3580, but may be implemented to make CP variants in any Cas9 sequence, either at CP sites that correspond to these positions, or at other CP sites entirely. This description is not meant to limit the specific CP sites in any way. Virtually any CP site may be used to form a CP-Cas9 variant.

In some embodiments, a prime editor comprises a Cas9 functional variant that is of smaller molecular weight than a wild type SpCas9 protein. In some embodiments, a smaller-sized Cas9 functional variant may facilitate delivery to cells, e.g., by an expression vector, nanoparticle, or other means of delivery. In certain embodiments, a smaller-sized Cas9 functional variant is a Class 2 Type II Cas protein. In certain embodiments, a smaller-sized Cas9 functional variant is a Class 2 Type V Cas protein. In certain embodiments, a smaller-sized Cas9 functional variant is a Class 2 Type VI Cas protein.

In some embodiments, a prime editor comprises a SpCas9 that is 1368 amino acids in length and has a predicted molecular weight of 158 kilodaltons. In some embodiments, a prime editor comprises a Cas9 functional variant or functional fragment that is less than 1300 amino acids, less than 1290 amino acids, than less than 1280 amino acids, less than 1270 amino acids, less than 1260 amino acid, less than 1250 amino acids, less than 1240 amino acids, less than 1230 amino acids, less than 1220 amino acids, less than 1210 amino acids, less than 1200 amino acids, less than 1190 amino acids, less than 1180 amino acids, less than 1170 amino acids, less than 1160 amino acids, less than 1150 amino acids, less than 1140 amino acids, less than 1130 amino acids, less than 1120 amino acids, less than 1110 amino acids, less than 1100 amino acids, less than 1050 amino acids, less than 1000 amino acids, less than 950 amino acids, less than 900 amino acids, less than 850 amino acids, less than 800 amino acids, less than 750 amino acids, less than 700 amino acids, less than 650 amino acids, less than 600 amino acids, less than 550 amino acids, or less than 500 amino acids, but at least larger than about 400 amino acids and retaining the one or more functions, e.g., DNA binding function, of the Cas9 protein.

In some embodiments, the Cas protein may include any CRISPR associated protein, including but not limited to, Cas12a, Cas12b1, Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof, and preferably comprising a nickase mutation (e.g., a mutation corresponding to the D10A mutation of the wild type Cas9 polypeptide of SEQ ID NO: 3580). In various other embodiments, the polypeptide domain having DNA binding activity can be any of the following proteins: a Cas9, a Cas12a (Cpf1), a Cas12e (CasX), a Cas12d (CasY), a Cas12b1 (C201), a Cas13a (C2c2), a Cas12c (C2c3), a GeoCas9, a CjCas9, a Cas12g, a Cas12h, a Cas12i, a Cas13b, a Cas13c, a Cas13d, a Cas14, a Csn2, an xCas9, an SpCas9-NG, a circularly permuted Cas9, or an Argonaute (Ago) domain, or a functional variant or fragment thereof.

### Exemplary Cas Proteins and Nomenclature are Shown in Table 44 Below:

TABLE 44

| Legacy nomenclature | Current nomenclature |
|---|---|
| type II CRISPR-Cas enzymes | |
| Cas9 | same |
| type V CRISPR-Cas enzymes | |
| Cpf1 | Cas12a |
| CasX | Cas12e |
| C2c1 | Cas12b1 |
| Cas12b2 | same |
| C2c3 | Cas12c |
| CasY | Cas12d |
| C2c4 | same |
| C2c8 | same |
| C2c5 | same |
| C2c10 | same |
| C2c9 | same |
| type VI CRISPR-Cas enzymes | |
| C2c2 | Cas13a |
| Cas13d | same |
| C2c7 | Cas13c |
| C2c6 | Cas13b |

In some embodiments, a prime editor as described herein may comprise a Cas12a (Cpf1) polypeptide or functional variants thereof. In some embodiments, the Cas12a polypeptide comprises a mutation that reduces or abolishes the endonuclease domain of the Cas12a polypeptide. In some embodiments, the Cas12a polypeptide is a Cas12a nickase. In some embodiments, the Cas protein comprises an amino acid sequence that comprises at least about 50%, 60%, 70%, 80%, 90%, 91%, 92%, 93%, 94%, 95%, 96%, 97%, 98%, 99%, or 100% sequence identity to a naturally occurring Cas12a polypeptide.

In some embodiments, a prime editor comprises a Cas protein that is a Cas12b (C2c1) or a Cas12c (C2c3) polypeptide. In some embodiments, the Cas protein comprises an amino acid sequence that comprises at least about 50%, 60%, 70%, 80%, 90%, 91%, 92%, 93%, 94%, 95%, 96%, 97%, 98%, 99%, or 100% sequence identity to a naturally occurring Cas12b (C2c1) or Cas12c (C2c3) protein. In some embodiments, the Cas protein is a Cas12b nickase or a Cas12c nickase. In some embodiments, the Cas protein is a Cas12e, a Cas12d, a Cas13, Cas14a, Cas14b, Cas14c, Cas14d, Cas14e, Cas14f, Cas14g, Cas14h, Cas14u, or a Casq polypeptide. In some embodiments, the Cas protein comprises an amino acid sequence that comprises at least about 50%, 60%, 70%, 80%, 90%, 91%, 92%, 93%, 94%, 95%, 96%, 97%, 98%, 99%, or 100% sequence identity to a naturally-occurring Cas12e, Cas12d, Cas13, Cas14a, Cas14b, Cas14c, Cas14d, Cas14e, Cas14f, Cas14g, Cas14h, Cas14u, or Cas q protein. In some embodiments, the Cas protein is a Cas12e, Cas12d, Cas13, or Cas q nickase.

### Flap Endonuclease

In some embodiments, a prime editor further comprises additional polypeptide components, for example, a flap endonuclease (FEN, e.g., FEN1). In some embodiments, the flap endonuclease excises the 5′ single stranded DNA of the edit strand of the target gene and assists incorporation of the intended nucleotide edit into the target gene. In some embodiments, the FEN is linked or fused to another component. In some embodiments, the FEN is provided in trans, for example, as a separate polypeptide or polynucleotide encoding the FEN.

In some embodiments, a prime editor or prime editing composition comprises a flap nuclease. In some embodiments, the flap nuclease is a FEN1, or any FEN1 functional variant, functional mutant, or functional fragment thereof. In some embodiments, the flap nuclease is a TREX2, EXO1, or any other flap nuclease known in the art, or any functional variant, functional mutant, or functional fragment thereof. In some embodiments, the flap nuclease has an amino acid sequence that is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to any of the flap nucleases described herein or known in the art.

### Nuclear Localization Sequences

In some embodiments, a prime editor further comprises one or more nuclear localization sequence (NLS). In some embodiments, the NLS helps promote translocation of a protein into the cell nucleus. In some embodiments, a prime editor comprises a fusion protein, e.g., a fusion protein comprising a DNA binding domain and a DNA polymerase, that comprises one or more NLSs. In some embodiments, one or more polypeptides of the prime editor are fused to or linked to one or more NLSs. In some embodiments, the prime editor comprises a DNA binding domain and a DNA polymerase domain that are provided in trans, wherein the DNA binding domain and/or the DNA polymerase domain is fused or linked to one or more NLSs.

In certain embodiments, a prime editor or prime editing composition comprises at least one NLS. In some embodiments, a prime editor or prime editing composition comprises at least two NLSs. In embodiments with at least two NLSs, the NLSs can be the same NLS, or they can be different NLSs.

In some instances, a prime editor may further comprise at least one nuclear localization sequence (NLS). In some cases, a prime editor may further comprise one NLS. In some cases, a prime editor may further comprise two NLSs. In other cases, a prime editor may further comprise three NLSs. In one case, a primer editor may further comprise more than 4, 5, 6, 7, 8, 9 or 10 NLSs.

In addition, the NLSs may be expressed as part of a prime editor or prime editing composition. In some embodiments, a NLS can be positioned almost anywhere in a protein's amino acid sequence, and generally comprises a short sequence of three or more or four or more amino acids. The location of the NLS fusion can be at the N-terminus, the C-terminus, or positioned anywhere within a sequence of a prime editor or a component thereof (e.g., inserted between the DNA-binding domain and the DNA polymerase domain of a prime editor fusion protein, between the DNA binding domain and a linker sequence, between a DNA polymerase and a linker sequence, between two linker sequences of a prime editor fusion protein or a component thereof, in either N-terminus to C-terminus or C-terminus to N-terminus order). In some embodiments, a prime editor is a fusion protein that comprises an NLS at the N terminus. In some embodiments, a prime editor is a fusion protein that comprises an NLS at the C terminus. In some embodiments, a prime editor is a fusion protein that comprises at least one NLS at both the N terminus and the C terminus. In some embodiments, the prime editor is a fusion protein that comprises two NLSs at the N terminus and/or the C terminus.

Any NLSs that are known in the art are also contemplated herein. The NLSs may be any naturally occurring NLS, or any non-naturally occurring NLS (e.g., an NLS with one or more mutations relative to a wild-type NLS). In some embodiments, the one or more NLSs of a prime editor comprise bipartite NLSs. In some embodiments, a nuclear localization signal (NLS) is predominantly basic. In some embodiments, the one or more NLSs of a prime editor are rich in lysine and arginine residues. In some embodiments, the one or more NLSs of a prime editor comprise proline residues. In some embodiments, a nuclear localization signal (NLS) comprises the sequence MDSLLMNRRKFLYQFKNVRWAKGRRETYLC (SEQ ID NO: 3610), MKRTADGSEFESPKKKRKV (SEQ ID NO: 3609), KRTADGSEFEPKKKRKV (SEQ ID NO: 3773), NLSKRPAAIKKAGQAKKKK (SEQ ID NO: 3774), RQRRNELKRSF, or NQSSNFGPMKGGNFGGRSSGPYGGGGQYFAKPRNQGGY (SEQ ID NO: 3775).

In some embodiments, a NLS is a monopartite NLS. For example, in some embodiments, a NLS is a SV40 large T antigen NLS (PKKKRKV (SEQ ID NO: 3608)). In some embodiments, a NLS is a bipartite NLS. In some embodiments, a bipartite NLS comprises two basic domains separated by a linker comprising a variable number of amino acids. In some embodiments, a NLS is a bipartite NLS. In some embodiments, a bipartite NLS consists of two basic domains separated by a linker comprising a variable number of amino acids. In some embodiments, the linker amino acid sequence comprises the sequence (KRXXXXXXXXXXKKKL, SEQ ID NO: 3776), wherein X is any amino acid. In some embodiments, the NLS comprises a nucleoplasmin NLS sequence KRPAATKKAGQAKKKK (SEQ ID NO: 3777). In some embodiments, a NLS is a noncanonical sequences such as M9 of the hnRNP A1 protein, the influenza virus nucleoprotein NLS, and the yeast Gal4 protein NLS.

Other non-limiting examples of NLS sequences are provided in Table 45 below.

TABLE 45
Exemplary nuclear localization sequences

| Description | Sequence | SEQ ID NO: |
|---|---|---|
| NLS of SV40 | PKKKRKV | 3608 |
| Large T-AG | | |
| NLS | MKRTADGSEFESPKKKRKV | 3609 |
| NLS | MDSLLMNRRKFLYQFKNVRWAKGRRETYLC | 3610 |
| NLS of | AVKRPAATKKAGQAKKKKLD | 3611 |
| Nucleoplasmin | | |
| NLS of EGL-13 | MSRRRKANPTKLSENAKKLAKEVEN | 3612 |
| NLS of C-Myc | PAAKRVKLD | 3613 |
| NLS of Tus- | KLKIKRPVK | 3614 |
| protein | | |
| NLS of polyoma | VSRKRPRP | 3615 |
| large T-AG | | |
| NLS of Hepatitis | EGAPPAKRAR | 3616 |
| D virus antigen | | |
| NLS of murine | PPQPKKKPLDGE | 3617 |
| p53 | | |
| C-terminal NLS | SGGSKRTADGSEFEPKKKRKV | 3618 |
| and linker for | | |
| exemplary PE | | |
| fusio protein | | |

### Additional Prime Editor Components

A prime editor described herein may comprise additional functional domains, for example, one or more domains that modify the folding, solubility, or charge of the prime editor. In some instances, the prime editor may comprise a solubility-enhancement (SET) domain.

In some embodiments, a split intein comprises two halves of an intein protein, which may be referred to as a N-terminal half of an intein, or intein-N, and a C-terminal half of an intein, or intein-C, respectively. In some embodiments, the intein-N and the intein-C may each be fused to a protein domain (the N-terminal and the C-terminal exteins). The exteins can be any protein or polypeptides, for example, any prime editor polypeptide component. In some embodiments, the intein-N and intein-C of a split intein can associate non-covalently to form an active intein and catalyze a trans splicing reaction. In some embodiments, the trans splicing reaction excises the two intein sequences and links the two extein sequences with a peptide bond. As a result, the intein-N and the intein-C are spliced out, and a protein domain linked to the intein-N is fused to a protein domain linked to the intein-C essentially in same way as a contiguous intein does. In some embodiments, a split-intein is derived from a eukaryotic intein, a bacterial intein, or an archaeal intein. Preferably, the split intein so-derived will possess only the amino acid sequences essential for catalyzing trans-splicing reactions. In some embodiments, an intein-N or an intein-C further comprise one or more amino acid substitutions as compared to a wild type intein-N or wild type intein-C, for example, amino acid substitutions that enhances the trans-splicing activity of the split intein. In some embodiments, the intein-C comprises 4 to 7 contiguous amino acid residues, wherein at least 4 amino acids of which are from the last β-strand of the intein from which it was derived. In some embodiments, the split intein is derived from a Ssp DnaE intein, e.g., Synechocytis sp. PCC6803, or any intein or split intein known in the art, or any functional variants or fragments thereof.

In some embodiments, a prime editor comprises one or more epitope tags. Non-limiting examples of epitope tags include histidine (His) tags, V5 tags, FLAG tags, influenza hemagglutinin (HA) tags, Myc tags, VSV-G tags, thioredoxin (Trx) tags, biotin carboxylase carrier protein (BCCP) tags, myc-tags, calmodulin-tags, polyhistidine tags, also referred to as histidine tags or His-tags, maltose binding protein (MBP)-tags, nus-tags, glutathione-S-transferase (GST)-tags, green fluorescent protein (GFP)-tags, thioredoxin-tags, S-tags, Softags (e.g., Softag 1, Softag 3), strep-tags, biotin ligase tags, FlAsH tags, V5 tags, and SBP-tags. Additional suitable sequences will be apparent to those of skill in the art. In some embodiments, the fusion protein comprises one or more His tags.

In some embodiments, a prime editor comprises one or more polypeptide domains encoded by one or more reporter genes. Examples of reporter genes include, but are not limited to, glutathione-S-transferase (GST), horseradish peroxidase (HRP), chloramphenicol acetyltransferase (CAT), beta-galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP).

In some embodiments, a prime editor comprises one or more polypeptide domains that binds DNA molecules or binds other cellular molecules. Examples of binding proteins or domains include, but are not limited to, maltose binding protein (MBP), S-tag, Lex A DNA binding domain (DBD) fusions, GAL4 DNA binding domain fusions, and herpes simplex virus (HSV) BP16 protein fusions.

Polypeptides comprising components of a prime editor may be fused via peptide linkers, or may be provided in trans relative to each other. For example, a reverse transcriptase may be expressed, delivered, or otherwise provided as an individual component rather than as a part of a fusion protein with the DNA binding domain. In such cases, components of the prime editor may be associated through non-peptide linkages or co-localization functions. In some embodiments, a prime editor further comprises additional components capable of interacting with, associating with, or capable of recruiting other components of the prime editor or the prime editing system. For example, a prime editor may comprise an RNA-protein recruitment polypeptide that can associate with an RNA-protein recruitment RNA aptamer. In some embodiments, an RNA-protein recruitment polypeptide can recruit, or be recruited by, a specific RNA sequence. Non limiting examples of RNA-protein recruitment polypeptide and RNA aptamer pairs include a MS2 coat protein and a MS2 RNA hairpin, a PCP polypeptide and a PP7 RNA hairpin, a Com polypeptide and a Com RNA hairpin, a Ku protein and a telomerase Ku binding RNA motif, and a Sm7 protein and a telomerase Sm7 binding RNA motif. In some embodiments, the prime editor comprises a DNA binding domain fused or linked to an RNA-protein recruitment polypeptide. In some embodiments, the prime editor comprises a DNA polymerase domain fused or linked to an RNA-protein recruitment polypeptide. In some embodiments, the DNA binding domain and the DNA polymerase domain fused to the RNA-protein recruitment polypeptide, or the DNA binding domain fused to the RNA-protein recruitment polypeptide and the DNA polymerase domain are co-localized by the corresponding RNA-protein recruitment RNA aptamer of the RNA-protein recruitment polypeptide. In some embodiments, the corresponding RNA-protein recruitment RNA aptamer is fused or linked to a portion of the PEgRNA. For example, an MS2 coat protein may be fused or linked to the DNA polymerase and a MS2 hairpin installed on the PEgRNA for co-localization of the DNA polymerase and the RNA-guided DNA binding domain (e.g., a Cas9 nickase).

In some embodiments, a prime editor comprises a polypeptide domain, an MS2 coat protein (MCP or MS2cp), that recognizes an MS2 hairpin. In some embodiments, the nucleotide sequence of the MS2 hairpin (or equivalently referred to as the “MS2 aptamer”) is: GCCAACATGAGGATCACCCATGTCTGCAGGGCC (SEQ ID NO: 3619). In some embodiments, the amino acid sequence of the MCP is:

(SEQ ID NO: 3620)
GSASNFTQFVLVDNGGTGDVTVAPSNFANGVAEWISSNSRSQAYKVTCS
VRQSSAQNRKYTIKVEVPKVATQTVGGEELPVAGWRSYLNMELTIPIFA
TNSDCELIVKAMQGLLKDGNPIPSAIA ANSGIY.

In certain embodiments, components of a prime editor are directly fused to each other. In certain embodiments, components of a prime editor are associated to each other via a linker.

As used herein, a linker can be any chemical group or a molecule linking two molecules or moieties, e.g., a DNA binding domain and a polymerase domain of a prime editor. In some embodiments, a linker is an organic molecule, group, polymer, or chemical moiety. In some embodiments, the linker comprises a non-peptide moiety. The linker may be as simple as a covalent bond, or it may be a polymeric linker many atoms in length, for example, a polynucleotide sequence. In certain embodiments, the linker is a covalent bond (e.g., a carbon-carbon bond, disulfide bond, carbon-heteroatom bond, etc.).

In certain embodiments, two or more components of a prime editor are linked to each other by a peptide linker. In some embodiments, a peptide linker is 5-100 amino acids in length, for example, 2, 3, 4, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. In some embodiments, the peptide linker is 16 amino acids in length, 24 amino acids in length, 64 amino acids in length, or 96 amino acids in length.

In some embodiments, the linker comprises the amino acid sequence (GGGGS)n (SEQ ID NO: 3627), (G)n (SEQ ID NO: 3628), (EAAAK)n (SEQ ID NO: 3629), (GGS)n (SEQ ID NO: 3630), (SGGS)n (SEQ ID NO: 3631), (XP)n (SEQ ID NO: 3632), or any combination thereof, wherein n is independently an integer between 1 and 30, and wherein X is any amino acid. In some embodiments, the linker comprises the amino acid sequence (GGS)n (SEQ ID NO: 3630), wherein n is 1, 3, or 7. In some embodiments, the linker comprises the amino acid sequence SGSETPGTSESATPES (SEQ ID NO: 3633). In some embodiments, the linker comprises the amino acid sequence SGGSSGGSSGSETPGTSESATPESSGGSSGGS (SEQ ID NO: 3634). In some embodiments, the linker comprises the amino acid sequence SGGSSGGSSGSETPGTSESATPESSGGSSGGSS (SEQ ID NO: 3635). In some embodiments, the linker comprises the amino acid sequence SGGSGGSGGS (SEQ ID NO: 3636). In some embodiments, the linker comprises the amino acid sequence SGGS (SEQ ID NO: 3637). In other embodiments, the linker comprises the amino acid sequence SGGSSGGSSGSETPGTSESATPESAGSYPYDVPDYAGSAAPAAKKKKLDGSGSGGSSGG S (SEQ ID NO: 3638).

In certain embodiments, two or more components of a prime editor are linked to each other by a non-peptide linker. In some embodiments, the linker is a carbon-nitrogen bond of an amide linkage. In certain embodiments, the linker is a cyclic or acyclic, substituted or unsubstituted, branched or unbranched aliphatic or heteroaliphatic linker. In certain embodiments, the linker is polymeric (e.g., polyethylene, polyethylene glycol, polyamide, polyester, etc.). In certain embodiments, the linker comprises a monomer, dimer, or polymer of aminoalkanoic acid. In certain embodiments, the linker comprises an aminoalkanoic acid (e.g., glycine, ethanoic acid, alanine, beta-alanine, 3-aminopropanoic acid, 4-aminobutanoic acid, 5-pentanoic acid, etc.). In certain embodiments, the linker comprises a monomer, dimer, or polymer of aminohexanoic acid (Ahx). In certain embodiments, the linker is based on a carbocyclic moiety (e.g., cyclopentane, cyclohexane). In other embodiments, the linker comprises a polyethylene glycol moiety (PEG). In certain embodiments, the linker comprises an aryl or heteroaryl moiety. In certain embodiments, the linker is based on a phenyl ring. The linker may include functionalized moieties to facilitate attachment of a nucleophile (e.g., thiol, amino) from the peptide to the linker. Any electrophile may be used as part of the linker. Exemplary electrophiles include, but are not limited to, activated esters, activated amides, Michael acceptors, alkyl halides, aryl halides, acyl halides, and isothiocyanates.

Components of a prime editor may be connected to each other in any order. In some embodiments, the DNA binding domain and the DNA polymerase domain of a prime editor may be fused to form a fusion protein, or may be joined by a peptide or protein linker, in any order from the N terminus to the C terminus. In some embodiments, a prime editor comprises a DNA binding domain fused or linked to the C-terminal end of a DNA polymerase domain. In some embodiments, a prime editor comprises a DNA binding domain fused or linked to the N-terminal end of a DNA polymerase domain. In some embodiments, the prime editor comprises a fusion protein comprising the structure NH2-[DNA binding domain]-[DNA polymerase]-CooH; or NH2-[DNA polymerase]-[DNA binding domain]-COOH, wherein each instance of “]-[” indicates the presence of an optional linker sequence. In some embodiments, a prime editor comprises a fusion protein and a DNA polymerase domain provided in trans, wherein the fusion protein comprises the structure NH2-[DNA binding domain]-[RNA-protein recruitment polypeptide]-COOH. In some embodiments, a prime editor comprises a fusion protein and a DNA binding domain provided in trans, wherein the fusion protein comprises the structure NH2-[DNA polymerase domain]-[RNA-protein recruitment polypeptide]-COOH.

In some embodiments, a prime editor fusion protein, a polypeptide component of a prime editor, or a polynucleotide encoding the prime editor fusion protein or polypeptide component, may be split into an N-terminal half and a C-terminal half or polypeptides that encode the N-terminal half and the C terminal half, and provided to a target DNA in a cell separately. For example, in certain embodiments, a prime editor fusion protein may be split into a N-terminal and a C-terminal half for separate delivery in AAV vectors, and subsequently translated and colocalized in a target cell to reform the complete polypeptide or prime editor protein. In such cases, separate halves of a protein or a fusion protein may each comprise a split-intein to facilitate colocalization and reformation of the complete protein or fusion protein by the mechanism of intein facilitated trans splicing. In some embodiments, a prime editor comprises a N-terminal half fused to an intein-N, and a C-terminal half fused to an intein-C, or polynucleotides or vectors (e.g., AAV vectors) encoding each thereof. When delivered and/or expressed in a target cell, the intein-N and the intein-C can be excised via protein trans-splicing, resulting in a complete prime editor fusion protein in the target cell.

The amino acid sequence of an exemplary prime editor (PE) fusion protein and its individual components is shown in Table 46. Table 47 also includes the amino acid sequence of an exemplary prime editor (PE) fusion protein and its individual components.

In some embodiments, a prime editing composition comprises a fusion protein comprising a DNA binding domain (e.g., Cas9(H840A)) and a reverse transcriptase (e.g., a variant M-MLV RT) having the following structure: [NLS]-[Cas9(H840A)]-[linker]-[M-MLV_RT(D200N)(T330P)(L603W)(T306K)(W313F)], and one or more desired PEgRNAs. In some embodiments, the prime editing composition comprises a prime editor fusion protein that has the amino acid sequence of SEQ ID NO: 3621.

In various embodiments, a prime editor fusion protein comprises an amino acid sequence that is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to the exemplary PE fusion protein provided below, or any of the prime editor fusion sequences described herein or known in the art.

TABLE 46
Amino acid sequence of exemplary PE and its individual components

| DESCRIPTION | SEQUENCE |
|---|---|
| PE FUSION | MKRTADGSEFESPKKKRKVDKKYSIGLDIGTNSVGWAVITDEYKVPSKKFK |
| PROTEIN | VLGNTDRHSIKKNLIGALLFDSGETAEATRLKRTARRRYTRRKNRICYLQ |
| CAS9(H840A)- | EIFSNEMAKVDDSFFHRLEESFLVEEDKKHERHPIFGNIVDEVAYHEKYPT |
| MMLV_RT | IYHLRKKLVDSTDKADLRLIYLALAHMIKFRGHFLIEGDLNPDNSDVDKL |
| D200N T330P | FIQLVQTYNQLFEENPINASGVDAKAILSARLSKSRRLENLIAQLPGEKKN |
| L603W T306K | GLFGNLIALSLGLTPNFKSNFDLAEDAKLQLSKDTYDDDLDNLLAQIGDQ |
| W313F | YADLFLAAKNLSDAILLSDILRVNTEITKAPLSASMIKRYDEHHQDLTLLK |
| | ALVRQQLPEKYKEIFFDQSKNGYAGYIDGGASQEEFYKFIKPILEKMDGT |
| | EELLVKLNREDLLRKQRTFDNGSIPHQIHLGELHAILRRQEDFYPFLKDN |
| | REKIEKILTFRIPYYVGPLARGNSRFAWMTRKSEETITPWNFEEVVDKGAS |
| | AQSFIERMTNFDKNLPNEKVLPKHSLLYEYFTVYNELTKVKYVTEGMRK |
| | PAFLSGEQKKAIVDLLFKTNRKVTVKQLKEDYFKKIECFDSVEISGVEDR |
| | FNASLGTYHDLLKIIKDKDFLDNEENEDILEDIVLTLTLFEDREMIEERLKT |
| | YAHLFDDKVMKQLKRRRYTGWGRLSRKLINGIRDKQSGKTILDFLKSDG |
| | FANRNFMQLIHDDSLTFKEDIQKAQVSGQGDSLHEHIANLAGSPAIKKGIL |
| | QTVKVVDELVKVMGRHKPENIVIEMARENQTTQKGQKNSRERMKRIEE |
| | GIKELGSQILKEHPVENTQLQNEKLYLYYLQNGRDMYVDQELDINRLSDY |
| | DVDAIVPQSFLKDDSIDNKVLTRSDKNRGKSDNVPSEEVVKKMKNYWRQ |
| | LLNAKLITQRKFDNLTKAERGGLSELDKAGFIKRQLVETRQITKHVAQIL |
| | DSRMNTKYDENDKLIREVKVITLKSKLVSDFRKDFQFYKVREINNYHHAH |
| | DAYLNAVVGTALIKKYPKLESEFVYGDYKVYDVRKMIAKSEQEIGKATAK |
| | YFFYSNIMNFFKTEITLANGEIRKRPLIETNGETGEIVWDKGRDFATVRKV |
| | LSMPQVNIVKKTEVQTGGFSKESILPKRNSDKLIARKKDWDPKKYGGFD |
| | SPTVAYSVLVVAKVEKGKSKKLKSVKELLGITIMERSSFEKNPIDFLEAKG |
| | YKEVKKDLIIKLPKYSLFELENGRKRMLASAGELQKGNELALPSKYVNFL |
| | YLASHYEKLKGSPEDNEQKQLFVEQHKHYLDEIIEQISEFSKRVILADANL |
| | DKVLSAYNKHRDKPIREQAENIIHLFTLTNLGAPAAFKYFDTTIDRKRYTS |
| | TKEVLDATLIHQSITGLYETRIDLSQLGGDSGGSSGGSSGSETPGTSESATP |
| | ESSGGSSGGSSTLNIEDEYRLHETSKEPDVSLGSTWLSDFPQAWAETGGMGLAVR |
| | QAPLIIPLKATSTPVSIKQYPMSQEARLGIKPHIQRLLDQGILVPCQSPWNTPLLPVK |
| | KPGTNDYRPVQDLREVNKRVEDIHPTVPNPYNLLSGLPPSHQWYTVLDLKDAFFC |
| | LRLHPTSQPLFAFEWRDPEMGISGQLTWTRLPQGFKNSPTLFNEALHRDLADFRI |
| | QHPDLILLQYVDDLLLAATSELDCQQGTRALLQTLGNLGYRASAKKAQICQKQVKY |
| | LGYLLKEGQRWLTEARKETVMGQPTPKTPRQLREFLGKAGFCRLFIPGFAEMAAP |
| | LYPLTKPGTLFNWGPDQQKAYQEIKQALLTAPALGLPDLTKPFELFVDEKQGYAK |
| | GVLTQKLGPWRRPVAYLSKKLDPVAAGWPPCLRMVAAIAVLTKDAGKLTMGQPLV |
| | ILAPHAVEALVKQPPDRWLSNARMTHYQALLLDTDRVQFGPVVALNPATLLPLPEE |
| | GLQHNCLDILAEAHGTRPDLTDQPLPDADHTWYTDGSSLLQEGQRKAGAAVTTET |
| | EVIWAKALPAGTSAQRAELIALTQALKMAEGKKLNVYTDSRYAFATAHIHGEIYRRRG |
| | WLTSEGKEIKNKDEILALLKALFLPKRLSIIHCPGHQKGHSAEARGNRMADQAARK |
| | AAITETPDTSTLLIENSSPSGGSKRTADGSEFEPKKKRKV (SEQ ID NO: 3621) |
| | KEY: |
| | NUCLEAR LOCALIZATION SEQUENCE (NLS) |
| | CAS9(H840A) |
| | 33-AMINO ACID LINKER |
| | M-MLV REVERSE TRANSCRIPTASE |
| N- TERMINAL | MKRTADGSEFESPKKKRKV (SEQ ID NO: 3609) |
| NLS | |
| CAS9 (H840A) | DKKYSIGLDIGTNSVGWAVITDEYKVPSKKFKVLGNTDRHSIKKNLIGALLFDS |
| (MET MINUS) | GETAEATRLKRTARRRYTRRKNRICYLQEIFSNEMAKVDDSFFHRLEESFLVEE |
| | DKKHERHPIFGNIVDEVAYHEKYPTIYHLRKKLVDSTDKADLRLIYLALAHMI |
| | KFRGHFLIEGDLNPDNSDVDKLFIQLVQTYNQLFEENPINASGVDAKAILSARL |
| | SKSRRLENLIAQLPGEKKNGLFGNLIALSLGLTPNFKSNFDLAEDAKLQLSKDT |
| | YDDDLDNLLAQIGDQYADLFLAAKNLSDAILLSDILRVNTEITKAPLSASMIKR |
| | YDEHHQDLTLLKALVRQQLPEKYKEIFFDQSKNGYAGYIDGGASQEEFYKFIK |
| | PILEKMDGTEELLVKLNREDLLRKQRTFDNGSIPHQIHLGELHAILRRQEDFYP |
| | FLKDNREKIEKILTFRIPYYVGPLARGNSRFAWMTRKSEETITPWNFEEVVDKG |
| | ASAQSFIERMTNFDKNLPNEKVLPKHSLLYEYFTVYNELTKVKYVTEGMRKP |
| | AFLSGEQKKAIVDLLFKTNRKVTVKQLKEDYFKKIECFDSVEISGVEDRFNAS |
| | LGTYHDLLKIIKDKDFLDNEENEDILEDIVLTLTLFEDREMIEERLKTYAHLFDD |
| | KVMKQLKRRRYTGWGRLSRKLINGIRDKQSGKTILDFLKSDGFANRNFMQLI |
| | HDDSLTFKEDIQKAQVSGQGDSLHEHIANLAGSPAIKKGILQTVKVVDELVKV |
| | MGRHKPENIVIEMARENQTTQKGQKNSRERMKRIEEGIKELGSQILKEHPVEN |
| | TQLQNEKLYLYYLQNGRDMYVDQELDINRLSDYDVDAIVPQSFLKDDSIDNK |
| | VLTRSDKNRGKSDNVPSEEVVKKMKNYWRQLLNAKLITQRKFDNLTKAERG |
| | GLSELDKAGFIKRQLVETRQITKHVAQILDSRMNTKYDENDKLIREVKVITLKS |
| | KLVSDFRKDFQFYKVREINNYHHAHDAYLNAVVGTALIKKYPKLESEFVYGD |
| | YKVYDVRKMIAKSEQEIGKATAKYFFYSNIMNFFKTEITLANGEIRKRPLIETN |
| | GETGEIVWDKGRDFATVRKVLSMPQVNIVKKTEVQTGGFSKESILPKRNSDKL |
| | IARKKDWDPKKYGGFDSPTVAYSVLVVAKVEKGKSKKLKSVKELLGITIMERS |
| | SFEKNPIDFLEAKGYKEVKKDLIIKLPKYSLFELENGRKRMLASAGELQKGNE |
| | LALPSKYVNFLYLASHYEKLKGSPEDNEQKQLFVEQHKHYLDEIIEQISEFSKR |
| | VILADANLDKVLSAYNKHRDKPIREQAENIIHLFTLTNLGAPAAFKYFDTTIDR |
| | KRYTSTKEVLDATLIHQSITGLYETRIDLSQLGGD (SEQ ID NO: 3582) |
| LINKER | SGGSSGGSSGSETPGTSESATPESSGGSSGGSS (SEQ ID NO: 3635) |
| BETWEEN | |
| CAS9 | |
| DOMAIN AND | |
| RT DOMAIN | |
| (33 AMINO | |
| ACIDS) | |
| MMLV_RT | TLNIEDEYRLHETSKEPDVSLGSTWLSDFPQAWAETGGMGLAVRQAPLIIPLK |
| D200N | ATSTPVSIKQYPMSQEARLGIKPHIQRLLDQGILVPCQSPWNTPLLPVKKPGTN |
| T330P | DYRPVQDLREVNKRVEDIHPTVPNPYNLLSGLPPSHQWYTVLDLKDAFFCLR |
| L603W | LHPTSQPLFAFEWRDPEMGISGQLTWTRLPQGFKNSPTLFNEALHRDLADFRI |
| T306K | QHPDLILLQYVDDLLLAATSELDCQQGTRALLQTLGNLGYRASAKKAQICQK |
| W313F | QVKYLGYLLKEGQRWLTEARKETVMGQPTPKTPRQLREFLGKAGFCRLFIPG |
| | FAEMAAPLYPLTKPGTLFNWGPDQQKAYQEIKQALLTAPALGLPDLTKPFELF |
| | VDEKQGYAKGVLTQKLGPWRRPVAYLSKKLDPVAAGWPPCLRMVAAIAVLT |
| | KDAGKLTMGQPLVILAPHAVEALVKQPPDRWLSNARMTHYQALLLDTDRVQ |
| | FGPVVALNPATLLPLPEEGLQHNCLDILAEAHGTRPDLTDQPLPDADHTWYTD |
| | GSSLLQEGQRKAGAAVTTETEVIWAKALPAGTSAQRAELIALTQALKMAEGK |
| | KLNVYTDSRYAFATAHIHGEIYRRRGWLTSEGKEIKNKDEILALLKALFLPKRL |
| | SIIHCPGHQKGHSAEARGNRMADQAARKAAITETPDTSTLLIENSSP (SEQ ID NO: 3579) |
| C-TERMINAL | SGGSKRTADGSEFEPKKKRKV (SEQ ID NO: 3618) |
| NLS | |

TABLE 47
Amino acid sequence of exemplary PE and its individual components

| DESCRIPTION | SEQUENCE |
|---|---|
| Exemplary prime editor | MKRTADGSEFESPKKKRKVDKKYSIGLDIGTNSVGWAVITDEYKVPS |
| [NLS]- | KKFKVLGNTDRHSIKKNLIGALLFDSGETAEATRLKRTARRRYTRR |
| [Cas9((R220K) | KNRICYLQEIFSNEMAKVDDSFFHRLEESFLVEEDKKHERHPIFGNI |
| (R393K) (H839A)]- | VDEVAYHEKYPTIYHLRKKLVDSTDKADLRLIYLALAHMIKFRGHF |
| [linker]- | LIEGDLNPDNSDVDKLFIQLVQTYNQLFEENPINASGVDAKAILSARL |
| [MMLV_RT(D200N) | SKSRKLENLIAQLPGEKKNGLFGNLIALSLGLTPNFKSNFDLAEDAK |
| (T330P)(L603W)(T306K) | LQLSKDTYDDDLDNLLAQIGDQYADLFLAAKNLSDAILLSDILRVNT |
| (W313F)]- | EITKAPLSASMIKRYDEHHQDLTLLKALVRQQLPEKYKEIFFDQSKN |
| [NLS] | GYAGYIDGGASQEEFYKFIKPILEKMDGTEELLVKLKREDLLRKQR |
| | TFDNGSIPHQIHLGELHAILRRQEDFYPFLKDNREKIEKILTFRIPYY |
| | VGPLARGNSRFAWMTRKSEETITPWNFEEVVDKGASAQSFIERMTN |
| | FDKNLPNEKVLPKHSLLYEYFTVYNELTKVKYVTEGMRKPAFLSGE |
| | QKKAIVDLLFKTNRKVTVKQLKEDYFKKIECFDSVEISGVEDRFNAS |
| | LGTYHDLLKIIKDKDFLDNEENEDILEDIVLTLTLFEDREMIEERLKT |
| | YAHLFDDKVMKQLKRRRYTGWGRLSRKLINGIRDKQSGKTILDFL |
| | KSDGFANRNFMQLIHDDSLTFKEDIQKAQVSGQGDSLHEHIANLAG |
| | SPAIKKGILQTVKVVDELVKVMGRHKPENIVIEMARENQTTQKGQ |
| | KNSRERMKRIEEGIKELGSQILKEHPVENTQLQNEKLYLYYLQNGR |
| | DMYVDQELDINRLSDYDVDAIVPQSFLKDDSIDNKVLTRSDKNRGKS |
| | DNVPSEEVVKKMKNYWRQLLNAKLITQRKFDNLTKAERGGLSELD |
| | KAGFIKRQLVETRQITKHVAQILDSRMNTKYDENDKLIREVKVITL |
| | KSKLVSDFRKDFQFYKVREINNYHHAHDAYLNAVVGTALIKKYPKL |
| | ESEFVYGDYKVYDVRKMIAKSEQEIGKATAKYFFYSNIMNFFKTEIT |
| | LANGEIRKRPLIETNGETGEIVWDKGRDFATVRKVLSMPQVNIVKK |
| | TEVQTGGFSKESILPKRNSDKLIARKKDWDPKKYGGFDSPTVAYSV |
| | LVVAKVEKGKSKKLKSVKELLGITIMERSSFEKNPIDFLEAKGYKEV |
| | KKDLIIKLPKYSLFELENGRKRMLASAGELQKGNELALPSKYVNFL |
| | YLASHYEKLKGSPEDNEQKQLFVEQHKHYLDEIIEQISEFSKRVILA |
| | DANLDKVLSAYNKHRDKPIREQAENIIHLFTLTNLGAPAAFKYFDTT |
| | IDRKRYTSTKEVLDATLIHQSITGLYETRIDLSQLGGDSGGSSGGSKR |
| | TADGSEFESPKKKRKVSGGSSGGSTLNIEDEYRLHETSKEPDVSLGSTWL |
| | SDFPQAWAETGGMGLAVRQAPLIIPLKATSTPVSIKQYPMSQEARLGIKPHIQ |
| | RLLDQGILVPCQSPWNTPLLPVKKPGTNDYRPVQDLREVNKRVEDIHPTVP |
| | NPYNLLSGLPPSHQWYTVLDLKDAFFCLRLHPTSQPLFAFEWRDPEMGISG |
| | QLTWTRLPQGFKNSPTLFNEALHRDLADFRIQHPDLILLQYVDDLLLAATSE |
| | LDCQQGTRALLQTLGNLGYRASAKKAQICQKQVKYLGYLLKEGQRWLTEAR |
| | KETVMGQPTPKTPRQLREFLGKAGFCRLFIPGFAEMAAPLYPLTKPGTLFN |
| | WGPDQQKAYQEIKQALLTAPALGLPDLTKPFELFVDEKQGYAKGVLTQKL |
| | GPWRRPVAYLSKKLDPVAAGWPPCLRMVAAIAVLTKDAGKLTMGQPLVILA |
| | PHAVEALVKQPPDRWLSNARMTHYQALLLDTDRVQFGPVVALNPATLLPLP |
| | EEGLQHNCLDILAEAHGTRPDLTDQPLPDADHTWYTDGSSLLQEGQRKAG |
| | AAVTTETEVIWAKALPAGTSAQRAELIALTQALKMAEGKKLNVYTDSRYAFAT |
| | AHIHGEIYRRRGWLTSEGKEIKNKDEILALLKALFLPKRLSIIHCPGHQKGHS |
| | AEARGNRMADQAARKAAITETPDTSTLLIENSSPSGGSKRTADGSEFESPKKK |
| | RKVGSGPAAKRVKLD (SEQ ID NO: 3622) |
| | KEY: |
| | N-terminal bipartiteSV40NLS |
| | CAS9(R221K N394K H840A) |
| | SGGSx2-met-bpSV40NLS-SGGSx2 LINKER |
| | M-MLV D200N T306K W313F T330P L603W REVERSE TRANSCRIPTASE |
| | C-terminal linker-NLS1 |
| | C-terminal linker-NLS2 |
| -N-terminal | MKRTADGSEFESPKKKRKV (SEQ ID NO: 3609) |
| bpSV40NLS | |
| -CAS9 (R221K | DKKYSIGLDIGTNSVGWAVITDEYKVPSKKFKVLGNTDRHSIKKNLIGA |
| N394K H840A) | LLFDSGETAEATRLKRTARRRYTRRKNRICYLQEIFSNEMAKVDDSFFH |
| | RLEESFLVEEDKKHERHPIFGNIVDEVAYHEKYPTIYHLRKKLVDSTDK |
| | ADLRLIYLALAHMIKFRGHFLIEGDLNPDNSDVDKLFIQLVQTYNQLFEE |
| | NPINASGVDAKAILSARLSKSRKLENLIAQLPGEKKNGLFGNLIALSLGL |
| | TPNFKSNFDLAEDAKLQLSKDTYDDDLDNLLAQIGDQYADLFLAAKNL |
| | SDAILLSDILRVNTEITKAPLSASMIKRYDEHHQDLTLLKALVRQQLPEK |
| | YKEIFFDQSKNGYAGYIDGGASQEEFYKFIKPILEKMDGTEELLVKLKRE |
| | DLLRKQRTFDNGSIPHQIHLGELHAILRRQEDFYPFLKDNREKIEKILTFR |
| | IPYYVGPLARGNSRFAWMTRKSEETITPWNFEEVVDKGASAQSFIERMT |
| | NFDKNLPNEKVLPKHSLLYEYFTVYNELTKVKYVTEGMRKPAFLSGEQ |
| | KKAIVDLLFKTNRKVTVKQLKEDYFKKIECFDSVEISGVEDRFNASLGT |
| | YHDLLKIIKDKDFLDNEENEDILEDIVLTLTLFEDREMIEERLKTYAHLFD |
| | DKVMKQLKRRRYTGWGRLSRKLINGIRDKQSGKTILDFLKSDGFANRN |
| | FMQLIHDDSLTFKEDIQKAQVSGQGDSLHEHIANLAGSPAIKKGILQTVK |
| | VVDELVKVMGRHKPENIVIEMARENQTTQKGQKNSRERMKRIEEGIKE |
| | LGSQILKEHPVENTQLQNEKLYLYYLQNGRDMYVDQELDINRLSDYDV |
| | DAIVPQSFLKDDSIDNKVLTRSDKNRGKSDNVPSEEVVKKMKNYWRQL |
| | LNAKLITQRKFDNLTKAERGGLSELDKAGFIKRQLVETRQITKHVAQIL |
| | DSRMNTKYDENDKLIREVKVITLKSKLVSDFRKDFQFYKVREINNYHH |
| | AHDAYLNAVVGTALIKKYPKLESEFVYGDYKVYDVRKMIAKSEQEIGK |
| | ATAKYFFYSNIMNFFKTEITLANGEIRKRPLIETNGETGEIVWDKGRDFA |
| | TVRKVLSMPQVNIVKKTEVQTGGFSKESILPKRNSDKLIARKKDWDPKK |
| | YGGFDSPTVAYSVLVVAKVEKGKSKKLKSVKELLGITIMERSSFEKNPI |
| | DFLEAKGYKEVKKDLIIKLPKYSLFELENGRKRMLASAGELQKGNELAL |
| | PSKYVNFLYLASHYEKLKGSPEDNEQKQLFVEQHKHYLDEIIEQISEFSK |
| | RVILADANLDKVLSAYNKHRDKPIREQAENIIHLFTLTNLGAPAAFKYF |
| | DTTIDRKRYTSTKEVLDATLIHQSITGLYETRIDLSQLGGD (SEQ ID NO: |
| | 3623) |
| SGGSx2- | SGGSSGGSKRTADGSEFESPKKKRKVSGGSSGGS (SEQ ID NO: 3624) |
| bpSV40NLS-SGGSx2 | |
| linker | |
| -MMLV_RT D200N | TLNIEDEYRLHETSKEPDVSLGSTWLSDFPQAWAETGGMGLAVRQAPLI |
| T330P L603W T306K | IPLKATSTPVSIKQYPMSQEARLGIKPHIQRLLDQGILVPCQSPWNTPLLP |
| W313F | VKKPGTNDYRPVQDLREVNKRVEDIHPTVPNPYNLLSGLPPSHQWYTV |
| | LDLKDAFFCLRLHPTSQPLFAFEWRDPEMGISGQLTWTRLPQGFKNSPT |
| | LFNEALHRDLADFRIQHPDLILLQYVDDLLLAATSELDCQQGTRALLQT |
| | LGNLGYRASAKKAQICQKQVKYLGYLLKEGQRWLTEARKETVMGQPT |
| | PKTPRQLREFLGKAGFCRLFIPGFAEMAAPLYPLTKPGTLFNWGPDQQK |
| | AYQEIKQALLTAPALGLPDLTKPFELFVDEKQGYAKGVLTQKLGPWRR |
| | PVAYLSKKLDPVAAGWPPCLRMVAAIAVLTKDAGKLTMGQPLVILAPH |
| | AVEALVKQPPDRWLSNARMTHYQALLLDTDRVQFGPVVALNPATLLP |
| | LPEEGLQHNCLDILAEAHGTRPDLTDQPLPDADHTWYTDGSSLLQEGQ |
| | RKAGAAVTTETEVIWAKALPAGTSAQRAELIALTQALKMAEGKKLNV |
| | YTDSRYAFATAHIHGEIYRRRGWLTSEGKEIKNKDEILALLKALFLPKRL |
| | SIIHCPGHQKGHSAEARGNRMADQAARKAAITETPDTSTLLIENSSP |
| | (SEQ ID NO: 3579) |
| C-terminal linker-NLS | SGGSKRTADGSEFESPKKKRKV (SEQ ID NO: 3625) |
| C-terminal linker-NLS2 | GSGPAAKRVKLD (SEQ ID NO: 3626) |

### PEgRNA for Editing of DMPK Gene

The term “prime editing guide RNA”, or “PEgRNA”, refers to a guide polynucleotide that comprises one or more intended nucleotide edits for incorporation into the target double stranded DNA. In some embodiments, the PEgRNA associates with and directs a prime editor to incorporate the one or more intended nucleotide edits into the target gene via prime editing.

In some embodiments, a PEgRNA comprises a spacer that is complementary or substantially complementary to a search target sequence on a target strand of the target gene. In some embodiments, the PEgRNA comprises a gRNA core that associates with a DNA binding domain, e.g., a CRISPR-Cas protein domain, of a prime editor. In some embodiments, the PEgRNA comprises an editing template. In some embodiments, the PEgRNA comprises a primer binding site (PBS). In some embodiments, a PEgRNA comprises an extension arm that comprises an editing template and a primer binding site (PBS).

Dual prime editing involves two different PEgRNAs each complexed with a prime editor. In some embodiments, the prime editor is the same for each of the PEgRNA-prime editor complexes. In some embodiments, the prime editor is different for each of the PEgRNA-prime editor complexes. In some embodiments, each of the two PEgRNAs comprises a region of complementarity to a distinct search target sequence of the double stranded target DNA, wherein the two distinct search target sequences are on the two complementary strands of the double stranded target DNA. In some embodiments, the two PEgRNAs each can direct a prime editor to initiate the prime editing process on the two complementary strands of the double stranded target DNA. In some embodiments, each of the two PEgRNAs comprises a spacer complementary to a separate search target sequence. In some embodiments, each of the two PEgRNAs anneals with a separate search target sequence through its spacer.

In some embodiments, a first PEgRNA comprises a first spacer complementary to a first search target sequence on a first strand of a double stranded target DNA, e.g., a double stranded target gene. In the context of the first PEgRNA, the first strand of the double stranded target DNA may be referred to as a first target strand, and the complementary strand referred to as the first PAM strand. In some embodiments, a first PEgRNA comprises a first gRNA core. In some embodiments, a first PEgRNA comprises a first editing template. In certain embodiments, a first PEgRNA comprises a first primer binding site (PBS) that is complementary to a free 3′ end formed at the first nick site.

In some embodiments, a second PEgRNA comprises a second spacer complementary to a second search target sequence on a second strand of a double stranded target DNA, e.g., a double stranded target gene. In the context of the second PEgRNA, the second strand of the double stranded target DNA may be referred to as a second target strand, and the complementary strand referred to as the second PAM strand. In some embodiments, a second PEgRNA comprises a second gRNA core. In some embodiments, a second PEgRNA comprises a second editing template. In some embodiments, a second PEgRNA comprises a second primer binding site (PBS) that is complementary to a free 3′ end formed at the second nick site.

In certain embodiments, the first editing template of a first PEgRNA and the second editing template of a second PEgRNA comprise a region of complementarity to each other. In certain embodiments, the region of complementarity between the first editing template and the second editing template comprises a nucleotide sequence that is exogenous to the double stranded target DNA or target gene. In certain embodiments, the exogenous sequence may be a marker, expression tag, barcode or regulatory sequence. In certain embodiments, the region of complementarity between the first editing template and the second editing template comprises a nucleotide sequence that is at least partially identical to a sequence in the double stranded target DNA or target gene. In certain embodiments, the region of complementarity between the first editing template and the second editing template comprises a nucleotide sequence that is at least partially identical to a sequence in the IND.

In certain embodiments, the first editing template of a first PEgRNA and the second editing template of a second PEgRNA do not comprise a region of complementarity to each other. In certain embodiments, the first editing template of a first PEgRNA comprises region of identity to a sequence on the first target strand (or the first strand), and the second editing template comprises a region of identity to a sequence on the second target strand (or the second strand). In certain embodiments, the first editing template of a first PEgRNA comprises a region of identity to a sequence on the first target strand immediately adjacent to and outside the IND. In certain embodiments, the second editing template of a second PEgRNA comprises a region of identity to a sequence on the second target strand immediately adjacent to and outside the IND. In some embodiments, an editing template comprises one or more intended nucleotide edits to be incorporated in the double stranded target DNA, e.g., the DMPK gene, by prime editing. In some embodiments, incorporation of the newly synthesized single stranded DNA encoded by the editing template results in incorporation of one or more intended nucleotide edit in the double stranded target DNA, e.g., the DMPK gene, compared to the endogenous sequence of the double stranded target gene. For example, in some embodiments, the one or more intended nucleotide edits comprises deletion, insertion, and/or substitution of one or more nucleotides compared to the endogenous sequence of the double stranded target gene, e.g., the DMPK gene. In some embodiments, the one or more intended nucleotide edits comprises deletion of an array of tri-nucleotide repeats compared to the endogenous sequence of the double stranded target gene, e.g., the DMPK gene. In some embodiments, the one or more intended nucleotide edits comprises deletion of an array of CTG repeats compared to the endogenous sequence of the double stranded target gene, e.g., the DMPK gene. In some embodiments, the one or more intended nucleotide edits comprises deletion of an array of tri-nucleotide repeats, e.g., an array of CTG repeats, and insertion of one or more exogenous sequences compared to the endogenous sequence of the double stranded target gene, e.g., the DMPK gene. In some embodiments, the one or more intended nucleotide edits comprises deletion of a portion of an array of tri-nucleotide repeats, e.g., an array of CTG repeats, and optionally insertion of one or more exogenous sequences compared to the endogenous sequence of the double stranded target gene, e.g., the DMPK gene. In some embodiments, the one or more intended nucleotide edits comprises deletion of 1-3, 1-5, 1-10, 1-15, 1-20, 1-25, 1-30, 1-35, 1-40, 1-45, 1-50, 1-55, 1-60, 1-75 or more tri-nucleotide repeats compared to the endogenous sequence of the double stranded target gene, e.g., the DMPK gene. In some embodiments, the one or more intended nucleotide edits comprises deletion of 1-3, 1-5, 1-10, 1-15, 1-20, 1-25, 1-30, 1-35, 1-40, 1-45, 1-50, 1-55, 1-60, 1-75 or more tri-nucleotide repeats compared to the endogenous sequence of the double stranded target gene, e.g., the DMPK gene. In some embodiments, the one or more intended nucleotide edits comprises deletion of 5-10, 5-15, 5-20, 5-25, 5-30, 5-35, 5-40, 5-50, 5-60, 5-70, 5-80, 5-90, 5-100, 20-30, 20-40, 20-50, 20-60, 20-70, 20-80, 20-90, 20-100, 35-40, 35-50, 35-60, 35-70, 35-80, 35-90, 35-100, 50-70, 50-80, 50-90, 50-100, 50-150, 50-200, 50-250, 50-300, 50-350, 50-400, 50-450, 50-500, 50-550, 50-600, 50-650, 50-700, 50-750, 50-800, 50-850, 50-900, 50-950, 50-1000, 50-1050, 50-1100, 50-1150, 50-1200, 50-1250, 50-1300, 50-1350, 50-1400, 50-1450, 50-1500, 100-150, 100-200, 100-250, 100-300, 100-350, 100-400, 100-450, 100-500, 100-550, 100-600, 100-650, 100-700, 100-750, 100-800, 100-850, 100-900, 100-950, 100-1000, 100-1050, 100-1100, 100-1150, 100-1200, 100-1250, 100-1300, 100-1350, 100-1400, 100-1450, 100-1500, 150-200, 150-250, 150-300, 150-350, 150-400, 150-450, 150-500, 150-550, 150-600, 150-650, 150-700, 150-750, 150-800, 150-850, 150-900, 150-950, 150-1000, 150-1050, 150-1100, 150-1150, 150-1200, 150-1250, 150-1300, 150-1350, 150-1400, 150-1450, 150-1500, 200-250, 200-300, 200-350, 200-400, 200-450, 200-500, 200-550, 200-600, 200-650, 200-700, 200-750, 200-800, 200-850, 200-900, 200-950, 200-1000, 200-1050, 200-1100, 200-1150, 200-1200, 200-1250, 200-1300, 200-1350, 200-1400, 200-1450, 200-1500, 250-300, 250-350, 250-400, 250-450, 250-500, 250-550, 250-600, 250-650, 250-700, 250-750, 250-800, 250-850, 250-900, 250-950, 250-1000, 250-1050, 250-1100, 250-1150, 250-1200, 250-1250, 250-1300, 250-1350, 250-1400, 250-1450, 250-1500, 300-350, 300-400, 300-450, 300-500, 300-550, 300-600, 300-650, 300-700, 300-750, 300-800, 300-850, 300-900, 300-950, 300-1000, 300-1050, 300-1100, 300-1150, 300-1200, 300-1250, 300-1300, 300-1350, 300-1400, 300-1450, 300-1500, 400-450, 400-500, 400-550, 400-600, 400-650, 400-700, 400-750, 400-800, 400-850, 400-900, 400-950, 400-1000, 400-1050, 400-1100, 400-1150, 400-1200, 400-1250, 400-1300, 400-1350, 400-1400, 400-1450, 400-1500, 500-550, 500-600, 500-650, 500-700, 500-750, 500-800, 500-850, 500-900, 500-950, 500-1000, 500-1050, 500-1100, 500-1150, 500-1200, 500-1250, 500-1300, 500-1350, 500-1400, 500-1450, 500-1500, 600-650, 600-700, 600-750, 600-800, 600-850, 600-900, 600-950, 600-1000, 600-1050, 600-1100, 600-1150, 600-1200, 600-1250, 600-1300, 600-1350, 600-1400, 600-1450, 600-1500, 700-750, 700-800, 700-850, 700-900, 700-950, 700-1000, 700-1050, 700-1100, 700-1150, 700-1200, 700-1250, 700-1300, 700-1350, 700-1400, 700-1450, 700-1500, 800-850, 800-900, 800-950, 800-1000, 800-1050, 800-1100, 800-1150, 800-1200, 800-1250, 800-1300, 800-1350, 800-1400, 800-1450, 800-1500, 900-950, 900-1000, 900-1050, 900-1100, 900-1150, 900-1200, 900-1250, 900-1300, 900-1350, 900-1400, 900-1450, 900-1500, 1000-1050, 1000-1100, 1000-1150, 1000-1200, 1000-1250, 1000-1300, 1000-1350, 1000-1400, 1000-1450, 1000-1500, 1100-1200, 1100-1300, 1100-1400, 1100-1500, 1200-1300, 1200-1400, 1200-1500, 1300-1400, 1300-1500, or 1400-1500 tri-nucleotide repeats compared to the endogenous sequence of the double stranded target gene, e.g., the DMPK gene.

In some embodiments, the editing template is a template for an RNA-dependent DNA polymerase domain or polypeptide of the prime editor, for example, a reverse transcriptase domain. The reverse transcriptase editing template may also be referred to herein as an RT template, or RTT.

In certain embodiments, the extension arm comprises a primer binding site sequence (PBS) that can initiate target-primed DNA synthesis. In some embodiments, the PBS is complementary or substantially complementary to a free 3′ end on the edit strand of the target gene at a nick site generated by the prime editor. In some embodiments, the first PEgRNA comprises a first PBS that comprises a region of complementarity to the second strand of the double stranded target DNA or target gene. In some embodiments, the second PEgRNA comprises a second PBS that comprises a region of complementarity to the first strand of the double stranded target DNA or target gene. In some embodiments, the first PEgRNA comprises a first PBS that comprises a region of complementarity to the first spacer of the first PEgRNA. In some embodiments, the first PEgRNA comprises a first PBS that is at least partially complementary to the first spacer of the first PEgRNA. In some embodiments, the second PEgRNA comprises a second PBS that comprises a region of complementarity to the second spacer of the second PEgRNA. In some embodiments, the second PEgRNA comprises a second PBS that is at least partially complementary to the second spacer of the second PEgRNA.

In some embodiments, a PEgRNA includes only RNA nucleotides and forms an RNA polynucleotide. In some embodiments, a PEgRNA is a chimeric polynucleotide that includes both RNA and DNA nucleotides. For example, a PEgRNA can include DNA in the spacer, the gRNA core, or the extension arm. In some embodiments, a PEgRNA comprises DNA in the spacer. In some embodiments, the entire spacer of a PEgRNA is a DNA sequence. In some embodiments, the PEgRNA comprises DNA in the gRNA core, for example, in a stem region of the gRNA core. In some embodiments, the PEgRNA comprises DNA in the extension arm, for example, in the editing template. An editing template that comprises a DNA sequence may serve as a DNA synthesis template for a DNA polymerase in a prime editor, for example, a DNA-dependent DNA polymerase. Accordingly, the PEgRNA may be a chimeric polynucleotide that comprises RNA in the spacer, gRNA core, and/or the PBS sequences and DNA in the editing template.

Components of a PEgRNA may be arranged in a modular fashion. In some embodiments, the spacer, the primer binding site sequence (PBS) and the editing template, e.g., a reverse transcriptase template (RTT), can be interchangeably located in the 5′ portion of the PEgRNA, the 3′ portion of the PEgRNA, or in the middle of the gRNA core. In some embodiments, a PEgRNA comprises a PBS and an editing template sequence in 5′ to 3′ order. In some embodiments, the gRNA core of a PEgRNA of this disclosure may be located in between a spacer and an extension arm (i.e., the PBS and editing template) of the PEgRNA. In some embodiments, the gRNA core of a PEgRNA may be located at the 3′ end of a spacer. In some embodiments, the gRNA core of a PEgRNA may be located at the 5′ end of a spacer. In some embodiments, the gRNA core of a PEgRNA may be located at the 3′ end of an extension arm. In some embodiments, the gRNA core of a PEgRNA may be located at the 5′ end of an extension arm. In some embodiments, a PEgRNA comprises, from 5′ to 3′: a spacer, a gRNA core, and an extension arm. In some embodiments, a PEgRNA comprises, from 5′ to 3′: a spacer, a gRNA core, an editing template, and a PBS. In some embodiments, a PEgRNA comprises, from 5′ to 3′: an extension arm, a spacer, and a gRNA core. In some embodiments, the PEgRNA comprises, from 5′ to 3′: an editing template, a PBS, a spacer, and a gRNA core. In some embodiments, a first PEgRNA comprises a structure: 5′-[first spacer]-[first gRNA core]-[first editing template]-[first primer binding site sequence]-3′. In some embodiments, a first PEgRNA comprises a structure: 5′-[first editing template]-[first primer binding site sequence]-[first spacer]-[first gRNA core]-3′. In some embodiments, a second PEgRNA comprises a structure: 5′-[second spacer]-[second gRNA core]-[second editing template]-[second primer binding site sequence]-3′. In some embodiments, a second PEgRNA comprises a structure: 5′-[second editing template]-[second primer binding site sequence]-[second spacer]-[second gRNA core]-3′.

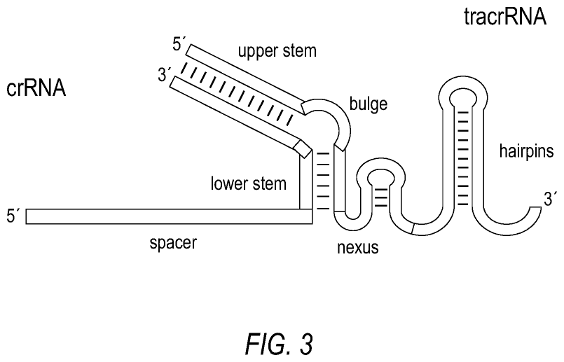

In some embodiments, a PEgRNA comprises a single polynucleotide molecule that comprises the spacer, the gRNA core, and the editing template. In some embodiments, a PEgRNA comprises a single polynucleotide molecule that comprises the spacer, the gRNA core, and the extension arm (i.e., a PBS and editing template). In some embodiments, a PEgRNA comprises multiple polynucleotide molecules, for example, two polynucleotide molecules. In some embodiments, a PEgRNA comprise a first polynucleotide molecule that comprises the spacer and a portion of the gRNA core, and a second polynucleotide molecule that comprises the rest of the gRNA core and the extension arm. In some embodiments, the gRNA core portion in the first polynucleotide molecule and the gRNA core portion in the second polynucleotide molecule are at least partly complementary to each other. In some embodiments, the PEgRNA may comprise a first polynucleotide comprising the spacer and a first portion of a gRNA core referred to as a crRNA. In some embodiments, the PEgRNA comprise a second polynucleotide comprising a second portion of the gRNA core and the extension arm, wherein the second portion of the gRNA core may also be referred to as a trans-activating crRNA, or tracr RNA. In some embodiments, the crRNA portion and the tracr RNA portion of the gRNA core are at least partially complementary to each other. In some embodiments, the partially complementary portions of the crRNA and the tracr RNA form a lower stem, a bulge, and an upper stem, as exemplified in FIG. 3.

In some embodiments, a first spacer comprises a region that has substantial complementarity to a first search target sequence on a first target strand, or first strand, of a double stranded target DNA, e.g., a DMPK gene. In some embodiments, the first spacer of a PEgRNA is identical or substantially identical to a protospacer sequence on the second strand of the target gene (except that the protospacer sequence comprises thymine and the spacer may comprise uracil). In some embodiments, the first spacer is at least about 70%, 75%, 80%, 85%, 90%, 95%, or 100% complementary to a first search target sequence in the target gene. In some embodiments, the first spacer is substantially complementary to the first search target sequence. In some embodiments, a second spacer comprises a region that has substantial complementarity to a second search target sequence on the second target strand, or the second strand, of a double stranded target DNA, e.g., a DMPK gene. In some embodiments, the second spacer of a PEgRNA is identical or substantially identical to a protospacer on the first strand of the target gene (except that the protospacer comprises thymine and the spacer may comprise uracil). In some embodiments, the second spacer is at least about 70%, 75%, 80%, 85%, 90%, 95%, or 100% complementary to a second search target sequence in the target gene. In some embodiments, the second spacer is substantially complementary to the second search target sequence.

In some embodiments, the length of the spacer varies from at least 10 nucleotides to 100 nucleotides. For examples, a spacer may be at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 21 nucleotides, at least 22 nucleotides, at least 23 nucleotides, at least 24 nucleotides, at least 25 nucleotides, at least 30 nucleotides, at least 40 nucleotides, at least 50 nucleotides, at least 60 nucleotides, at least 70 nucleotides, at least 80 nucleotides, at least 90 nucleotides, at least 100 nucleotides. In some embodiments, the spacer is 16 nucleotides, 17 nucleotides, 18 nucleotides, 19 nucleotides, 20 nucleotides, 21 nucleotides, 22 nucleotides, 23 nucleotides, 24 nucleotides, or 25 nucleotides in length. In some embodiments, the spacer is from 15 nucleotides to 30 nucleotides in length, 15 to 25 nucleotides in length, 17 to 23 nucleotides in length, 18 to 22 nucleotides in length, 10 to 20 nucleotides in length, 20 to 30 nucleotides in length, 30 to 40 nucleotides in length, 40 to 50 nucleotides in length, 50 to 60 nucleotides in length, 60 to 70 nucleotides in length, 70 to 80 nucleotides in length, or 90 nucleotides to 100 nucleotides in length. In some embodiments, the spacer is 20 nucleotides in length. In some embodiments, the spacer is 17 to 18 nucleotides in length. In some embodiments, the spacer is 21 to 22 nucleotides in length.

The extension arm of a first PEgRNA may be partially complementary to the spacer of the first PEgRNA. In some embodiments, the editing template (e.g., RTT) of a first PEgRNA is partially complementary to the spacer of the first PEgRNA. In some embodiments, the editing template (e.g., RTT) and the primer binding site (PBS) of the first PEgRNA are each partially complementary to the spacer of the first PEgRNA. The extension arm of a PEgRNA may comprise a primer binding site (PBS) and an editing template (e.g., an RTT). The extension arm of a second PEgRNA may be partially complementary to the spacer of the second PEgRNA. In some embodiments, the editing template (e.g., RTT) of a second PEgRNA is partially complementary to the spacer of the second PEgRNA. In some embodiments, the editing template (e.g., RTT) and the primer binding site (PBS) of the second PEgRNA are each partially complementary to the spacer of the second PEgRNA.

An extension arm of a PEgRNA may comprise a primer binding site sequence (also referred to as a primer binding site, PBS, or PBS sequence) that hybridizes with a free 3′ end of a single stranded DNA in the target gene (e.g., the DMPK gene) generated by nicking with a prime editor. The length of the PBS sequence may vary depending on, e.g., the prime editor components, the search target sequence and other components of the PEgRNA. In some embodiments, the length of the primer binding site (PBS) varies from at least 2 nucleotides to 50 nucleotides. For examples, a primer binding site (PBS) may be at least 2 nucleotides, at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 30 nucleotides, at least 40 nucleotides, or at least 50 nucleotides in length. In some embodiments, the PBS is at least 4 nucleotides in length. In some embodiments, the PBS is at least 6 nucleotides in length. In some embodiments, the PBS is about 4 to 12 nucleotides, about 6 to 12 nucleotides, about 8 to 12 nucleotides, about 10 to 12 nucleotides, 4 to 14 nucleotides, about 6 to 14 nucleotides, about 8 to 14 nucleotides, about 10 to 14 nucleotides, about 12 to 14 nucleotides, 4 to 16 nucleotides, about 6 to 16 nucleotides, about 8 to 16 nucleotides, about 10 to 16 nucleotides, about 6 to 18 nucleotides, about 6 to 20 nucleotides, about 8 to 20 nucleotides, about 10 to 20 nucleotides, about 12 to 20 nucleotides, about 14 to 20 nucleotides, about 16 to 20 nucleotides, or about 18 to 20 nucleotides in length. In some embodiments, the PBS is about 7 to 15 nucleotides in length. In some embodiments, the PBS is 6, 7, 8, 9, 10, 11, 12, 13, 14, 15, 16, 17, 18, 19, or 20 nucleotides in length. In some embodiments, the PBS is 8, 9, 10, 11, 12, 13, or 14 nucleotides in length. In some embodiments, the PBS is 3 to 19 nucleotides in length. In some embodiments, the PBS is 3 to 17 nucleotides in length. In some embodiments, the PBS is about 8 to 17 nucleotides in length. In some embodiments, the PBS is 8 to 9, 8 to 10, 8 to 11, or 8 to 12 nucleotides in length.

The PBS of a first PEgRNA may be complementary or substantially complementary to a DNA sequence in the second strand of the target gene. The PBS of a second PEgRNA may be complementary or substantially complementary to a DNA sequence in the first strand of the target gene. By annealing with the edit strand at a free hydroxy group, e.g., a free 3′ end generated by prime editor nicking, a PBS may initiate synthesis of a new single stranded DNA encoded by the editing template at the nick site. In some embodiments, a PBS is at least about 70%, 75%, 80%, 85%, 90%, 95%, or 100% complementary to a region of the edit strand of the target gene (e.g., the DMPK gene). In some embodiments, a PBS is perfectly complementary, or 100% complementary, to a region of the edit strand of the target gene (e.g., the DMPK gene).

An extension arm of a PEgRNA may comprise an editing template that serves as a DNA synthesis template for the DNA polymerase in a prime editor during prime editing.

The length of an editing template may vary depending on, e.g., the prime editor components, the search target sequence and other components of the PEgRNA. In some embodiments, the editing template serves as a DNA synthesis template for a reverse transcriptase, and the editing template is referred to as a reverse transcription editing template or simply reverse transcriptase template (RTT).

In some embodiments, the editing template (e.g., RTT) is 2, 3, 4, 5, 6, 7, 8, 9, 10, 11, 12, 13, 14, 15, 16, 17, 18, 19, 20, 21, 22, 23, 24, or 25 nucleotides in length. In some embodiments, the editing template is 12, 13, 14, 15, 16, 17, 18, 19, 20, 21, 22, 23, 24, or 25 nucleotides in length. In some embodiments, the editing template is 16, 17, 18, 19, 20, 21, 22, 23, 24, 25, 26, 27, 28, 29, 30, 31, 32, 33, 34, 35, 36, 37, 38, 39, or 40 nucleotides in length. In some embodiments, the editing template comprises 2, 3, 4, 5, 6, 7, 8, 9, 10, 11, 12, 13, 14, 15, 16, 17, 18, 19, 20, 21, 22, 23, 24, 25, 30, 35, 40, 45, 50 or more nucleotides. In some embodiments, the editing template comprises about 5 to 10, 5 to 15, 5 to 20, 5 to 25, 5 to 30, 5 to 35, 5 to 40, 5 to 45, 5 to 50, 5 to 55, 5 to 60, 5 to 65, 5 to 70, 5 to 75, 5 to 80, 5 to 85, 5 to 90, 5 to 95, 5 to 100, 5 to 110, 5 to 120, 5 to 130, 5 to 140, 5 to 150, 15 to 20, 15 to 25, 15 to 30, 15 to 35, 15 to 40, 15 to 45, 15 to 50, 15 to 55, 15 to 60, 15 to 65, 15 to 70, 15 to 75, 15 to 80, 15 to 85, 15 to 90, 15 to 95, 15 to 100, 15 to 110, 15 to 120, 15 to 130, 15 to 140, 15 to 150, 25 to 30, 25 to 35, 25 to 40, 25 to 45, 25 to 50, 25 to 55, 25 to 60, 25 to 65, 25 to 70, 25 to 75, 25 to 80, 25 to 85, 25 to 90, 25 to 95, 25 to 100, 25 to 110, 25 to 120, 25 to 130, 25 to 140, 25 to 150, 35 to 40, 35 to 45, 35 to 50, 35 to 55, 35 to 60, 35 to 65, 35 to 70, 35 to 75, 35 to 80, 35 to 85, 35 to 90, 35 to 95, 35 to 100, 35 to 110, 35 to 120, 35 to 130, 35 to 140, 35 to 150, 45 to 50, 45 to 55, 45 to 60, 45 to 65, 45 to 70, 45 to 75, 45 to 80, 45 to 85, 45 to 90, 45 to 95, 45 to 100, 45 to 110, 45 to 120, 45 to 130, 45 to 140, o45 to 150, 55 to 60, 55 to 65, 55 to 70, 55 to 75, 55 to 80, 55 to 85, 55 to 90, 55 to 95, 55 to 100, 55 to 110, 55 to 120, 55 to 130, 55 to 140, 55 to 150, 65 to 70, 65 to 75, 65 to 80, 65 to 85, 65 to 90, 65 to 95, 65 to 100, 65 to 110, 65 to 120, 65 to 130, 65 to 140, 65 to 150, 75 to 80, 75 to 85, 75 to 90, 75 to 95, 75 to 100, 75 to 110, 75 to 120, 75 to 130, 75 to 140, 75 to 150, 85 to 90, 85 to 95, 85 to 100, 85 to 110, 85 to 120, 85 to 130, 85 to 140, 85 to 150, 95 to 100, 95 to 110, 95 to 120, 95 to 130, 95 to 140, 95 to 150, 105 to 110, 105 to 120, 105 to 130, 105 to 140, 105 to 150, 115 to 120, 115 to 130, 115 to 140, 115 to 150, 125 to 130, 125 to 140, 125 to 150, 135 to 140, 135 to 150, or 145 to 150 nucleotides in length.

In some embodiments, the editing template comprises 10, 11, 12, 13, 14, 15, 16, 17, 18, 19, 20, 21, 22, 23, 24 or 25 nucleotides in length. In some embodiments, the editing template comprises 30, 35, 40, 50, 60, 70, 80, 90, or 100 nucleotides in length. In some embodiments, the editing template comprises no greater than 20, 25, 30, 35, 40, 50, 60, 70, 80, 90, or 100 nucleotides in length. In some embodiments, the editing template comprises a sufficient number of nucleotides to form a sufficiently stable duplex with a sequence on the double stranded target DNA. In some embodiments, the editing template comprises at least 10 polynucleotides. In some embodiments, the editing template comprises at least 15 polynucleotides. In some embodiments, the editing template comprises at least 20 polynucleotides.

RTT's may also include sequences unrelated to the endogenous sequence. This may be done, for example, to insert a readily-identifiable sequence to permit rapid determination of successful editing, or to improve editing efficiency by controlling insert length or GC content. In some embodiments, the editing template has a GC content of about 40%. In some embodiments, the editing template has a GC content of about 50%. In some embodiments, the editing template has a GC content of about 60%. In some embodiments, the editing template has a GC content of about 70%. In some embodiments, the editing template has a GC content of about 80%.

An intended nucleotide edit in an editing template of a PEgRNA may comprise various types of alterations as compared to the target gene sequence. In some embodiments, the nucleotide edit is a single nucleotide substitution as compared to the target gene sequence. In some embodiments, the nucleotide edit is a deletion as compared to the target gene sequence. In some embodiments, the nucleotide edit is an insertion as compared to the target gene sequence. In some embodiments, the editing template comprises one to ten intended nucleotide edits as compared to the target gene sequence. In some embodiments, the editing template comprises one or more intended nucleotide edits as compared to the target gene sequence. In some embodiments, the editing template comprises two or more intended nucleotide edits as compared to the target gene sequence. In some embodiments, the editing template comprises three or more intended nucleotide edits as compared to the target gene sequence. In some embodiments, the editing template comprises four or more, five or more, or six or more intended nucleotide edits as compared to the target gene sequence. In some embodiments, the editing template comprises two single nucleotide substitutions, insertions, deletions, or any combination thereof, as compared to the target gene sequence. In some embodiments, the editing template comprises three single nucleotide substitutions, insertions, deletions, or any combination thereof, as compared to the target gene sequence. In some embodiments, the editing template comprises four, five, or six single nucleotide substitutions, insertions, deletions, or any combination thereof, as compared to the target gene sequence. In some embodiments, a nucleotide substitution comprises an adenine (A)-to-thymine (T) substitution. In some embodiments, a nucleotide substitution comprises an A-to-guanine (G) substitution. In some embodiments, a nucleotide substitution comprises an A-to-cytosine (C) substitution. In some embodiments, a nucleotide substitution comprises a T-A substitution. In some embodiments, a nucleotide substitution comprises a T-G substitution. In some embodiments, a nucleotide substitution comprises a T-C substitution. In some embodiments, a nucleotide substitution comprises a G-to-A substitution. In some embodiments, a nucleotide substitution comprises a G-to-T substitution. In some embodiments, a nucleotide substitution comprises a G-to-C substitution. In some embodiments, a nucleotide substitution comprises a C-to-A substitution. In some embodiments, a nucleotide substitution comprises a C-to-T substitution. In some embodiments, a nucleotide substitution comprises a C-to-G substitution.

In some embodiments, a nucleotide insertion is at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, or at least 20 nucleotides in length. In some embodiments, a nucleotide insertion is from 1 to 2 nucleotides, from 1 to 3 nucleotides, from 1 to 4 nucleotides, from 1 to 5 nucleotides, form 2 to 5 nucleotides, from 3 to 5 nucleotides, from 3 to 6 nucleotides, from 3 to 8 nucleotides, from 4 to 9 nucleotides, from 5 to 10 nucleotides, from 6 to 11 nucleotides, from 7 to 12 nucleotides, from 8 to 13 nucleotides, from 9 to 14 nucleotides, from 10 to 15 nucleotides, from 11 to 16 nucleotides, from 12 to 17 nucleotides, from 13 to 18 nucleotides, from 14 to 19 nucleotides, from 15 to 20 nucleotides in length. In some embodiments, a nucleotide insertion is a single nucleotide insertion. In some embodiments, a nucleotide insertion comprises insertion of two nucleotides.

The editing template of a PEgRNA may comprise one or more intended nucleotide edits, compared to the DMPK gene to be edited. Position of the intended nucleotide edit(s) relevant to other components of the PEgRNA, or to particular nucleotides (e.g., mutations) in the DMPK target gene may vary. In some embodiments, the nucleotide edit is in a region of the PEgRNA corresponding to or homologous to the protospacer sequence. In some embodiments, the nucleotide edit is in a region of the PEgRNA corresponding to a region of the DMPK gene outside of the protospacer sequence.

In some embodiments, incorporation of the one or more intended nucleotide edits results in deletion of the inter-nick duplex (IND) from the double stranded target DNA, e.g., the DMPK gene. In some embodiments, the IND comprises a mutation compared to a wild type gene sequence, e.g., a wild type DMPK gene. In some embodiments, the IND comprises a mutation in the 3′ untranslated region of the DMPK gene as compared to a wild type DMPK gene. In some embodiments, the mutation is expansion of the number of CTG repeats compared to a wild type DMPK gene. In some embodiments, the IND is located between positions corresponding to positions 45770205 and 45770207 of human chromosome 19 as set forth in human genome research consortium human build 38 (GRCh38). In some embodiments, the IND is located between positions corresponding to positions 45770105 and 45770307 of human chromosome 19 as set forth in human genome research consortium human build 38 (GRCh38). In some embodiments, the IND is located between positions corresponding to positions 45770005 and 45770407 of human chromosome 19 as set forth in human genome research consortium human build 38 (GRCh38). In some embodiments, the IND is located between positions corresponding to positions 45776905 and 45770507 of human chromosome 19 as set forth in human genome research consortium human build 38 (GRCh38)

In some embodiments, the editing template comprises an adenine at the first nucleobase position (e.g., for a PEgRNA following 5′-spacer-gRNA core-RTT-PBS-3′ orientation, the 5′ most nucleobase is the “first base”). In some embodiments, the editing template comprises a guanine at the first nucleobase position (e.g., for a PEgRNA following 5′-spacer-gRNA core-RTT-PBS-3′ orientation, the 5′ most nucleobase is the “first base”). In some embodiments, the editing template comprises an uracil at the first nucleobase position (e.g., for a PEgRNA following 5′-spacer-gRNA core-RTT-PBS-3′ orientation, the 5′ most nucleobase is the “first base”). In some embodiments, the editing template comprises a cytosine at the first nucleobase position (e.g., for a PEgRNA following 5′-spacer-gRNA core-RTT-PBS-3′ orientation, the 5′ most nucleobase is the “first base”). In some embodiments, the editing template does not comprise a cytosine at the first nucleobase position (e.g., for a PEgRNA following 5′-spacer-gRNA core-RTT-PBS-3′ orientation, the 5′ most nucleobase is the “first base”).

A guide RNA core (also referred to herein as the gRNA core, gRNA scaffold, or gRNA backbone sequence) of a PEgRNA may contain a polynucleotide sequence that binds to a DNA binding domain (e.g., Cas9) of a prime editor. The gRNA core may interact with a prime editor as described herein, for example, by association with a DNA binding domain, such as a Cas9 nickase of the prime editor.

One of skill in the art will recognize that different prime editors having different DNA binding domains from different DNA binding proteins may require different gRNA core sequences specific to the DNA binding protein. In some embodiments, the gRNA core is capable of binding to a Cas9-based prime editor. In some embodiments, the gRNA core is capable of binding to a Cpf1-based prime editor. In some embodiments, the gRNA core is capable of binding to a Cas12b-based prime editor.

In some embodiments, the gRNA core comprises regions and secondary structures involved in binding with specific CRISPR Cas proteins. For example, in a Cas9 based prime editing system, the gRNA core of a PEgRNA may comprise one or more regions of a base paired “lower stem” adjacent to the spacer and a base paired “upper stem” following the lower stem, where the lower stem and upper stem may be connected by a “bulge” comprising unpaired RNAs. The gRNA core may further comprise a “nexus” distal from the spacer, followed by a hairpin structure, e.g., at the 3′ end, as exemplified in FIG. 3. In some embodiments, the gRNA core comprises modified nucleotides as compared to a wild type gRNA core in the lower stem, upper stem, and/or the hairpin. For example, nucleotides in the lower stem, upper stem, and/or the hairpin regions may be modified, deleted, or replaced. In some embodiments, RNA nucleotides in the lower stem, upper stem, and/or the hairpin regions may be replaced with one or more DNA sequences. In some embodiments, the gRNA core comprises unmodified or wild type RNA sequences in the nexus and/or the bulge regions. In some embodiments, the gRNA core does not include long stretches of A-T pairs, for example, a GUUUU-AAAAC pairing element. In some embodiments, the gRNA core is capable of binding to a SpCas9, and comprises the sequence: GUUUUAGAGCUAGAAAUAGCAAGUUAAAAUAAGGCUAGUCCGUUAUCAACUUGA AAAAGUGGCACCGAGUCGGUGC (SEQ ID NO: 3641); GUUUGAGAGCUAGAAAUAGCAAGUUUAAAUAAGGCUAGUCCGUUAUCAACUUGA AAAAGUGGGACCGAGUCGGUCC (SEQ ID NO: 3642), or GUUUAAGAGCUAUGCUGGAAACAGCAUAGCAAGUUUAAAUAAGGCUAGUCCGUU AUCAACUUGAAAAAGUGGCACCGAGUCGGUGC (SEQ ID NO: 3643). In some embodiments, the gRNA core comprises the sequence GUUUUAGAGCUAGAAAUAGCAAGUUAAAAUAAGGCUAGUCCGUUAUCAACUUGA AAAAGUGGCACCGAGUCGGUGC (SEQ ID NO: 3641). In some embodiments, the gRNA core is capable of binding to a SaCas9 and comprises the sequence GTTTTAGTACTCTGGAAACAGAATCTACTAAAACAAGGCAAAATGCCGTGT TTATCTCGTCAACTTGTTGGCGAGA (SEQ ID NO: 3644)). In some embodiments, the gRNA core is capable of binding to a SluCas9 and comprises the sequence GTTTTAGTACTCTGGAAACAGAATCTACTGAAACAAGACAATATGTCGTGTTTATCC CATCAATTTATTGGTGGGA (SEQ ID NO: 3645). Any gRNA core sequences known in the art are also contemplated in the prime editing compositions described herein. In some embodiments, one or more nucleotides in the gRNA core is DNA.

In some embodiments, a PEgRNA is produced by transcription from a template nucleotide, for example, a template plasmid. In some embodiments, a polynucleotide encoding the PEgRNA is appended with one or more additional nucleotides that improves PEgRNA expression, e.g., expression from a plasmid that encodes the PEgRNA or ngRNA. In some embodiments, a polynucleotide encoding a PEgRNA is appended with one or more additional nucleotides at the 5′ end or at the 3′ end. In some embodiments, the polynucleotide encoding the PEgRNA is appended with a guanine at the 5′ end, for example, if the first nucleotide at the 5′ end of the spacer is not a guanine. In some embodiments, a polynucleotide encoding the PEgRNA is appended with nucleotide sequence CACC at the 5′ end. In some embodiments, the polynucleotide encoding the PEgRNA is appended with additional nucleotide sequence TTTTTT, TTTTTTT, TTTTT, or TTTT at the 3′ end. In some embodiments, the PEgRNA comprises the appended nucleotides from the transcription template. In some embodiments, the PEgRNA or ngRNA further comprises one or more nucleotides at the 5′ end or the 3′ end in addition to spacer, PBS, and RTT sequences. in some embodiments, the PEgRNA or ngRNA further comprises a guanine at the 5′ end, for example, when the first nucleotide at the 5′ end of the spacer is not a guanine. In some embodiments, the PEgRNA or ngRNA further comprises nucleotide sequence CACC at the 5′ end. In some embodiments, the PEgRNA or ngRNA further comprises nucleotide sequence UUUUUUU, UUUUUU, UUUUU, or UUUU at the 3′ end.

A PEgRNA may also comprise optional modifiers, e.g., 3′ end modifier region and/or an 5′ end modifier region. In some embodiments, a PEgRNA comprises at least one nucleotide that is not part of a spacer, a gRNA core, or an extension arm. The optional sequence modifiers could be positioned within or between any of the other regions shown, and not limited to being located at the 3′ and 5′ ends. In certain embodiments, the PEgRNA comprises secondary RNA structure, such as, but not limited to, aptamers, hairpins, stem/loops, toeloops, and/or RNA-binding protein recruitment domains (e.g., the MS2 aptamer which recruits and binds to the MS2 coat protein (MS2cp)). In some embodiments, a PEgRNA comprises a short stretch of uracil at the 5′ end or the 3′ end. For example, in some embodiments, a PEgRNA comprising a 3′ extension arm comprises a “UUU” sequence at the 3′ end of the extension arm. In some embodiments, a PEgRNA comprises a toeloop sequence at the 3′ end. In some embodiments, the PEgRNA comprises a 3′ extension arm and a toeloop sequence at the 3′ end of the extension arm. In some embodiments, the PEgRNA comprises a 5′ extension arm and a toeloop sequence at the 5′ end of the extension arm. In some embodiments, the PEgRNA comprises a toeloop element having the sequence 5′-GAAANNNNN-3′, wherein N is any nucleobase. In some embodiments, the secondary RNA structure is positioned within the spacer. In some embodiments, the secondary structure is positioned within the extension arm. In some embodiments, the secondary structure is positioned within the gRNA core. In some embodiments, the secondary structure is positioned between the spacer and the gRNA core, between the gRNA core and the extension arm, or between the spacer and the extension arm. In some embodiments, the secondary structure is positioned between the PBS and the editing template. In some embodiments the secondary structure is positioned at the 3′ end or at the 5′ end of the PEgRNA. In some embodiments, the PEgRNA comprises a transcriptional termination signal at the 3′ end of the PEgRNA. In some embodiments, a PEgRNA comprises up to 50 nucleotides, up to 40 nucleotides, up to 30 nucleotides, up to 20 nucleotides, or up to 10 nucleotides of optional sequence modifiers at either or both of the 3′ and 5′ ends of the PEgRNA. In addition to secondary RNA structures, the PEgRNA may comprise a chemical linker or a poly(N) linker or tail, where “N” can be any nucleobase. In some embodiments, the chemical linker may function to prevent reverse transcription of the gRNA core.

A PEgRNA of this disclosure, in some embodiments, may include modified nucleotides, e.g., chemically modified DNA or RNA nucleobases, and may include one or more nucleobase analogs (e.g., modifications which might add functionality, such as temperature resilience). In some embodiments, PEgRNAs as described herein may be chemically modified. The phrase “chemical modifications,” as used herein, can include modifications which introduce chemistries which differ from those seen in naturally occurring DNA or RNAs, for example, covalent modifications such as the introduction of modified nucleotides, (e.g., nucleotide analogs, or the inclusion of pendant groups which are not naturally found in DNA or RNA molecules).

In some embodiments, the PEgRNAs provided in this disclosure may have undergone a chemical or biological modifications. Modifications may be made at any position within a PEgRNA, and may include modification to a nucleobase or to a phosphate backbone of the PEgRNA. In some embodiments, chemical modifications can be structure guided modifications. In some embodiments, a chemical modification is at the 5′ end and/or the 3′ end of a PEgRNA. In some embodiments, a chemical modification may be within the spacer, the extension arm, the editing template sequence, or the primer binding site of a PEgRNA. In some embodiments, a chemical modification may be within the spacer or the gRNA core of a PEgRNA. In some embodiments, a chemical modification may be within the 3′ most nucleotides of a PEgRNA. In some embodiments, a chemical modification may be within the 3′ most end of a PEgRNA. In some embodiments, a chemical modification may be within the 5′ most end of a PEgRNA. In some embodiments, a PEgRNA comprises 1, 2, 3, 4, 5, 6, 7, 8, 9, 10 or more chemically modified nucleotides at the 3′ end. In some embodiments, a PEgRNA comprises 1, 2, 3, 4, 5, 6, 7, 8, 9, 10 or more chemically modified nucleotides at the 5′ end. In some embodiments, a PEgRNA comprises 1, 2, 3, 4, or 5 or more chemically modified nucleotides at the 3′ end. In some embodiments, a PEgRNA comprises 1, 2, 3, 4, or 5 or more chemically modified nucleotides at the 5′ end. In some embodiments, a PEgRNA comprises 1, 2, or 3 or more chemically modified nucleotides at the 3′ end. In some embodiments, a PEgRNA comprises 1, 2, or 3 or more chemically modified nucleotides at the 5′ end.

In some embodiments, a PEgRNA comprises 1, 2, 3, 4, 5, 6, 7, 8, 9, 10 or more contiguous chemically modified nucleotides at the 3′ end. In some embodiments, a PEgRNA comprises 1, 2, 3, 4, 5, 6, 7, 8, 9, 10 or more contiguous chemically modified nucleotides at the 5′ end. In some embodiments, a PEgRNA comprises 1, 2, 3, 4, or 5 contiguous chemically modified nucleotides at the 3′ end. In some embodiments, a PEgRNA comprises 1, 2, 3, 4, or 5 contiguous chemically modified nucleotides at the 5′ end. In some embodiments, a PEgRNA comprises 1, 2, or 3 contiguous chemically modified nucleotides at the 3′ end. In some embodiments, a PEgRNA comprises 1, 2, or 3 contiguous chemically modified nucleotides at the 5′ end. In some embodiments, a PEgRNA comprises 3 contiguous chemically modified nucleotides at the 3′ end.

In some embodiments, a PEgRNA comprises 1, 2, 3, 4, 5, or more chemically modified nucleotides near the 3′ end. In some embodiments, a PEgRNA comprises 3 contiguous chemically modified nucleotides at the 3′ end. In some embodiments, a PEgRNA comprises 3 contiguous chemically modified nucleotides at the 5′ end. In some embodiments, a PEgRNA comprises 1, 2, 3, 4, 5, or more chemically modified nucleotides near the 3′ end. In some embodiments, a PEgRNA comprises 1, 2, 3, 4, 5, or more contiguous chemically modified nucleotides near the 3′ end. In some embodiments, a PEgRNA comprises 1, 2, 3, 4, 5, or more chemically modified nucleotides near the 3′ end, where the 3′ most nucleotide is not modified, and the 1, 2, 3, 4, 5, or more chemically modified nucleotides precede the 3′ most nucleotide in a 5′-to-3′ order. In some embodiments, a PEgRNA comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or more chemically modified nucleotides near the 3′ end, where the 3′ most nucleotide is not modified, and the 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or more chemically modified nucleotides precede the 3′ most nucleotide in a 5′-to-3′ order.

In some embodiments, a PEgRNA comprises one or more chemically modified nucleotides in the gRNA core. In some embodiments, the gRNA core of a PEgRNA may comprise one or more regions of a base paired lower stem, a base paired upper stem, where the lower stem and upper stem may be connected by a bulge comprising unpaired RNAs. The gRNA core may further comprise a nexus distal from the spacer. In some embodiments, the gRNA core comprises one or more chemically modified nucleotides in the lower stem, upper stem, and/or the hairpin regions. In some embodiments, all of the nucleotides in the lower stem, upper stem, and/or the hairpin regions are chemically modified.

A chemical modification to a PEgRNA can comprise a 2′-O-thionocarbamate-protected nucleoside phosphoramidite, a 2′-O-methyl (M), a 2′-O-methyl 3′phosphorothioate (MS), or a 2′-O-methyl 3′thioPACE (MSP), or any combination thereof. In some embodiments, a chemically modified PEgRNA can comprise a ′-O-methyl (M) RNA, a 2′-O-methyl 3′phosphorothioate (MS) RNA, a 2′-O-methyl 3′thioPACE (MSP) RNA, a 2′-F RNA, a phosphorothioate bond modification, any other chemical modifications known in the art, or any combination thereof. A chemical modification may also include, for example, the incorporation of non-nucleotide linkages or modified nucleotides into the PEgRNA (e.g., modifications to one or both of the 3′ and 5′ ends of a guide RNA molecule). Such modifications can include the addition of bases to an RNA sequence, complexing the RNA with an agent (e.g., a protein or a complementary nucleic acid molecule), and inclusion of elements which change the structure of an RNA molecule (e.g., which form secondary structures).

### Prime Editing Compositions

Disclosed herein, in some embodiments, are compositions, systems, and methods using a prime editing composition. The term “prime editing composition” or “prime editing system” refers to compositions involved in the method of prime editing as described herein. A prime editing composition may include a prime editor, e.g., a prime editor fusion protein, and a PEgRNA. A prime editing composition may further comprise additional elements. Components of a prime editing composition may be combined to form a complex for prime editing, or may be kept separately, e.g., for administration purposes.

In some embodiments, a prime editing composition comprises a first prime editing guide RNA (PEgRNA), a second PEgRNA, and a prime editor. In some embodiments, a prime editing composition comprises a first prime editing guide RNA (PEgRNA), a second PEgRNA, and a prime editor fusion protein complexed with the first PEgRNA and a prime editor fusion protein complexed with the second PEgRNA. In some embodiments, the prime editor fusion protein complexed with the first PEgRNA and the prime editor fusion protein complexed with the second PEgRNA are the identical prime editor fusion protein. In some embodiments, the prime editor fusion protein complexed with the first PEgRNA and the prime editor fusion protein complexed with the second PEgRNA are different prime editor fusion proteins.

In some embodiments, a prime editing composition comprises a first prime editing guide RNA (PEgRNA), a second PEgRNA, and a prime editor comprising a DNA binding domain and a DNA polymerase domain associated with each other through the first PEgRNA and/or the second PEgRNA. For example, the prime editing composition may comprise a prime editor comprising a DNA binding domain and a DNA polymerase domain linked to each other by an RNA-protein recruitment aptamer RNA sequence, which is linked to either or both of the first and second PEgRNAs. In some embodiments, the prime editing composition comprises a first PEgRNA, a second PEgRNA, and a prime editor comprising a DNA binding domain and a DNA polymerase domain, wherein the prime editor for both the first PEgRNA and the second PEgRNA are the same. In some embodiments, the prime editing composition comprises a first PEgRNA, a second PEgRNA, and a prime editor comprising a DNA binding domain and a DNA polymerase domain, wherein the prime editor for both the first PEgRNA and the second PEgRNA are different.

In some embodiments, a prime editing composition comprises a first PEgRNA, a second PEgRNA, and a polynucleotide, a polynucleotide construct, or a vector that encodes a prime editor fusion protein. In some embodiments, a prime editing composition comprises a first PEgRNA, a second PEgRNA, and one or more polynucleotides, one or more polynucleotide constructs, or one or more vectors that encode a prime editor comprising a DNA binding domain and a DNA polymerase domain. In some embodiments, a prime editing composition comprises a first PEgRNA, a second PEgRNA, and a polynucleotide, a polynucleotide construct, or a vector that encodes a prime editor fusion protein. In some embodiments, a prime editing composition comprises multiple polynucleotides, polynucleotide constructs, or vectors, each of which encodes one or more prime editing composition components. In some embodiments, the first PEgRNA and/or the second PEgRNA of a prime editing composition is associated with the DNA binding domain, e.g., a Cas9 nickase, of the prime editor. In some embodiments, the first PEgRNA and/or the second PEgRNA of a prime editing composition complexes with the DNA binding domain of a prime editor and directs the prime editor to the double stranded target DNA.

In some embodiments, a prime editing composition comprises one or more polynucleotides that encode prime editor components and/or the first PEgRNA and/or the second PEgRNAs. In some embodiments, a prime editing composition comprises a polynucleotide encoding a fusion protein comprising a DNA binding domain and a DNA polymerase domain. In some embodiments, a prime editing composition comprises (i) a polynucleotide encoding a fusion protein comprising a DNA binding domain and a DNA polymerase domain, (ii) a first PEgRNA or a polynucleotide encoding the first PEgRNA, and (iii) a second PEgRNA or a polynucleotide encoding the second PEgRNA. In some embodiments, a prime editing composition consists of (i) a polynucleotide encoding a fusion protein comprising a DNA binding domain and a DNA polymerase domain, (ii) a first PEgRNA or a polynucleotide encoding the first PEgRNA, and (iii) a second PEgRNA or a polynucleotide encoding the second PEgRNA. In some embodiments, a prime editing composition comprises (i) a polynucleotide encoding a DNA binding domain of a prime editor, e.g., a Cas9 nickase, (ii) a polynucleotide encoding a DNA polymerase domain of a prime editor, e.g., a reverse transcriptase, (iii) a first PEgRNA or a polynucleotide encoding the first PEgRNA, and (iv) a second PEgRNA or a polynucleotide encoding the second PEgRNA. In some embodiments, a prime editing composition consists of (i) a polynucleotide encoding a DNA binding domain of a prime editor, e.g., a Cas9 nickase, (ii) a polynucleotide encoding a DNA polymerase domain of a prime editor, e.g., a reverse transcriptase, (iii) a first PEgRNA or a polynucleotide encoding the first PEgRNA, and (iv) a second PEgRNA or a polynucleotide encoding the second PEgRNA.

In some embodiments, the polynucleotide encoding the DNA binding domain or the polynucleotide encoding the DNA polymerase domain further encodes an additional polypeptide domain, e.g., an RNA-protein recruitment domain, such as a MS2 coat protein domain. In some embodiments, a prime editing composition comprises (i) a polynucleotide encoding a N-terminal half of a prime editor fusion protein and an intein-N and (ii) a polynucleotide encoding a C-terminal half of a prime editor fusion protein and an intein-C. In some embodiments, a prime editing composition comprises (i) a polynucleotide encoding a N-terminal half of a prime editor fusion protein and an intein-N (ii) a polynucleotide encoding a C-terminal half of a prime editor fusion protein and an intein-C, (iii) a first PEgRNA or a polynucleotide encoding the first PEgRNA, and (iv) a second PEgRNA or a polynucleotide encoding the second PEgRNA. In some embodiments, a prime editing composition comprises (i) a polynucleotide encoding a N-terminal portion of a DNA binding domain and an intein-N, (ii) a polynucleotide encoding a C-terminal portion of the DNA binding domain, an intein-C, and a DNA polymerase domain. In some embodiments, the DNA binding domain is a Cas protein domain, e.g., a Cas9 nickase. In some embodiments, the prime editing composition comprises (i) a polynucleotide encoding a N-terminal portion of a DNA binding domain and an intein-N, (ii) a polynucleotide encoding a C-terminal portion of the DNA binding domain, an intein-C, and a DNA polymerase domain, (iii) a first PEgRNA or a polynucleotide encoding the first PEgRNA, and (iv) a second PEgRNA or a polynucleotide encoding the second PEgRNA.

The editing template of the first PEgRNA (the “first editing template”) and the editing template of the second PEgRNA (the “second editing template”) of a prime editing system may or may not have sequence complementarity to each other. In some embodiments, the first editing template has a region of complementarity or substantial complementarity to the second editing template. In some embodiments, the region of complementarity or substantial complementarity to the second editing template is 2, 3, 4, 5, 6, 7, 8, 9, 10, 11, 12, 13, 14, 15, 16, 17, 18, 19, 20, 21, 22, 23, 24, 25, 30, 35, 40, 45, 50 or more nucleotides in length. In some embodiments, the region of complementarity or substantial complementarity to the second editing template is about 5 to 10, 5 to 15, 5 to 20, 5 to 25, 5 to 30, 5 to 35, 5 to 40, 5 to 45, 5 to 50, 5 to 55, 5 to 60, 5 to 65, 5 to 70, 5 to 75, 5 to 80, 5 to 85, 5 to 90, 5 to 95, 5 to 100, 5 to 110, 5 to 120, 5 to 130, 5 to 140, 5 to 150, 15 to 20, 15 to 25, 15 to 30, 15 to 35, 15 to 40, 15 to 45, 15 to 50, 15 to 55, 15 to 60, 15 to 65, 15 to 70, 15 to 75, 15 to 80, 15 to 85, 15 to 90, 15 to 95, 15 to 100, 15 to 110, 15 to 120, 15 to 130, 15 to 140, 15 to 150, 25 to 30, 25 to 35, 25 to 40, 25 to 45, 25 to 50, 25 to 55, 25 to 60, 25 to 65, 25 to 70, 25 to 75, 25 to 80, 25 to 85, 25 to 90, 25 to 95, 25 to 100, 25 to 110, 25 to 120, 25 to 130, 25 to 140, 25 to 150, 35 to 40, 35 to 45, 35 to 50, 35 to 55, 35 to 60, 35 to 65, 35 to 70, 35 to 75, 35 to 80, 35 to 85, 35 to 90, 35 to 95, 35 to 100, 35 to 110, 35 to 120, 35 to 130, 35 to 140, 35 to 150, 45 to 50, 45 to 55, 45 to 60, 45 to 65, 45 to 70, 45 to 75, 45 to 80, 45 to 85, 45 to 90, 45 to 95, 45 to 100, 45 to 110, 45 to 120, 45 to 130, 45 to 140, o45 to 150, 55 to 60, 55 to 65, 55 to 70, 55 to 75, 55 to 80, 55 to 85, 55 to 90, 55 to 95, 55 to 100, 55 to 110, 55 to 120, 55 to 130, 55 to 140, 55 to 150, 65 to 70, 65 to 75, 65 to 80, 65 to 85, 65 to 90, 65 to 95, 65 to 100, 65 to 110, 65 to 120, 65 to 130, 65 to 140, 65 to 150, 75 to 80, 75 to 85, 75 to 90, 75 to 95, 75 to 100, 75 to 110, 75 to 120, 75 to 130, 75 to 140, 75 to 150, 85 to 90, 85 to 95, 85 to 100, 85 to 110, 85 to 120, 85 to 130, 85 to 140, 85 to 150, 95 to 100, 95 to 110, 95 to 120, 95 to 130, 95 to 140, 95 to 150, 105 to 110, 105 to 120, 105 to 130, 105 to 140, 105 to 150, 115 to 120, 115 to 130, 115 to 140, 115 to 150, 125 to 130, 125 to 140, 125 to 150, 135 to 140, 135 to 150, or 145 to 150 nucleotides in length. In some embodiments, the region of complementarity or substantial complementarity to the second editing template is 10, 11, 12, 13, 14, 15, 16, 17, 18, 19, 20, 21, 22, 23, 24 or 25 nucleotides in length. In some embodiments, the region of complementarity or substantial complementarity to the second editing template is 30, 35, 40, 50, 60, 70, 80, 90, or 100 nucleotides in length. In some embodiments, the region of complementarity or substantial complementarity to the second editing template is at most 20, 25, 30, 35, 40, 50, 60, 70, 80, 90, or 100 nucleotides in length. In some embodiments, the region of complementarity or substantial complementarity to the second editing template is at least 10 nucleotides in length. In some embodiments, the region of complementarity or substantial complementarity to the second editing template is at least 15 nucleotides in length. In some embodiments, the region of complementarity or substantial complementarity to the second editing template is at least 20 nucleotides in length.

In some embodiments, the first editing template has a region of complementarity to the second editing template and does not have a region of complementarity or substantial complementarity to the target double stranded DNA sequence. In some embodiments, the second editing template has a region of complementarity to the first editing template and does not have a region of complementarity or substantial complementarity to the target double stranded DNA sequence.

In some embodiments, the first editing template comprises a region of complementarity or substantial complementarity to the target double stranded DNA sequence. In some embodiments, the first editing template has a region of complementarity or substantial complementarity to the second editing template and has a region of complementarity or substantial complementarity to the target double stranded DNA sequence.

In some embodiments, the second editing template comprises a region of complementarity or substantial complementarity to the target double stranded DNA sequence. In some embodiments, the second editing template has a region of complementarity to the first editing template and has a region of complementarity or substantial complementarity to the target double stranded DNA sequence.

The region of complementarity or substantial complementarity of the first editing template to the double stranded target DNA sequence may or may not have the same length as the region of complementarity or substantial complementarity of the second editing template to the double stranded target DNA sequence.

In some embodiments, the region of complementarity or substantial complementarity of the first editing template to the target double stranded DNA sequence is about 5 to 10, 5 to 15, 5 to 20, 5 to 25, 5 to 30, 5 to 35, 5 to 40, 5 to 45, 5 to 50, 5 to 55, 5 to 60, 5 to 65, 5 to 70, 5 to 75, 5 to 80, 5 to 85, 5 to 90, 5 to 95, 5 to 100, 5 to 110, 5 to 120, 5 to 130, 5 to 140, 5 to 150, 5 to 175, 5 to 200, 5 to 225, 5 to 250, 5 to 275, 5 to 300, 5 to 325, 5 to 350, 5 to 375, 5 to 400, 5 to 425, 5 to 450, 5 to 475, 5 to 500, 10 to 15, 10 to 20, 10 to 25, 10 to 30, 10 to 35, 10 to 40, 10 to 45, 10 to 50, 10 to 55, 10 to 60, 10 to 65, 10 to 70, 10 to 75, 10 to 80, 10 to 85, 10 to 90, 10 to 95, 10 to 100, 10 to 110, 10 to 120, 10 to 130, 10 to 140, 10 to 150, 10 to 175, 10 to 200, 10 to 225, 10 to 250, 10 to 275, 10 to 300, 10 to 325, 10 to 350, 10 to 375, 10 to 400, 10 to 425, 10 to 450, 10 to 475, 10 to 500, 15 to 20, 15 to 25, 15 to 30, 15 to 35, 15 to 40, 15 to 45, 15 to 50, 15 to 55, 15 to 60, 15 to 65, 15 to 70, 15 to 75, 15 to 80, 15 to 85, 15 to 90, 15 to 95, 15 to 100, 15 to 110, 15 to 120, 15 to 130, 15 to 140, 15 to 150, 15 to 175, 15 to 200, 15 to 225, 15 to 250, 15 to 275, 15 to 300, 15 to 325, 15 to 350, 15 to 375, 15 to 400, 15 to 425, 15 to 450, 15 to 475, 15 to 500, 20 to 25, 20 to 30, 20 to 35, 20 to 40, 20 to 45, 20 to 50, 20 to 55, 20 to 60, 20 to 65, 20 to 70, 20 to 75, 20 to 80, 20 to 85, 20 to 90, 20 to 95, 20 to 100, 20 to 110, 20 to 120, 20 to 130, 20 to 140, 20 to 150, 20 to 175, 20 to 200, 20 to 225, 20 to 250, 20 to 275, 20 to 300, 20 to 325, 20 to 350, 20 to 375, 20 to 400, 20 to 425, 20 to 450, 20 to 475, 20 to 500, 30 to 35, 30 to 40, 30 to 45, 30 to 50, 30 to 55, 30 to 60, 30 to 65, 30 to 70, 30 to 75, 30 to 80, 30 to 85, 30 to 90, 30 to 95, 30 to 100, 30 to 110, 30 to 120, 30 to 130, 30 to 140, 30 to 150, 30 to 175, 30 to 200, 30 to 225, 30 to 250, 30 to 275, 30 to 300, 30 to 325, 30 to 350, 30 to 375, 30 to 400, 30 to 425, 30 to 450, 30 to 475, 30 to 500, 40 to 45, 40 to 50, 40 to 55, 40 to 60, 40 to 65, 40 to 70, 40 to 75, 40 to 80, 40 to 85, 40 to 90, 40 to 95, 40 to 100, 40 to 110, 40 to 120, 40 to 130, 40 to 140, 40 to 150, 40 to 175, 40 to 200, 40 to 225, 40 to 250, 40 to 275, 40 to 300, 40 to 325, 40 to 350, 40 to 375, 40 to 400, 40 to 425, 40 to 450, 40 to 475, 40 to 500, 50 to 55, 50 to 60, 50 to 65, 50 to 70, 50 to 75, 50 to 80, 50 to 85, 50 to 90, 50 to 95, 50 to 100, 50 to 110, 50 to 120, 50 to 130, 50 to 140, 50 to 150, 50 to 175, 50 to 200, 50 to 225, 50 to 250, 50 to 275, 50 to 300, 50 to 325, 50 to 350, 50 to 375, 50 to 400, 50 to 425, 50 to 450, 50 to 475, 50 to 500, 75 to 80, 75 to 85, 75 to 90, 75 to 95, 75 to 100, 75 to 110, 75 to 120, 75 to 130, 75 to 140, 75 to 150, 75 to 175, 75 to 200, 75 to 225, 75 to 250, 75 to 275, 75 to 300, 75 to 325, 75 to 350, 75 to 375, 75 to 400, 75 to 425, 75 to 450, 75 to 475, 75 to 500, 100 to 110, 100 to 120, 100 to 130, 100 to 140, 100 to 150, 100 to 175, 100 to 200, 100 to 225, 100 to 250, 100 to 275, 100 to 300, 100 to 325, 100 to 350, 100 to 375, 100 to 400, 100 to 425, 100 to 450, 100 to 475, 100 to 500, 125 to 150, 125 to 175, 125 to 200, 125 to 225, 125 to 250, 125 to 275, 125 to 300, 125 to 325, 125 to 350, 125 to 375, 125 to 400, 125 to 425, 125 to 450, 125 to 475, 125 to 500, 150 to 175, 150 to 200, 150 to 225, 150 to 250, 150 to 275, 150 to 300, 150 to 325, 150 to 350, 150 to 375, 150 to 400, 150 to 425, 150 to 450, 150 to 475, 150 to 500, 175 to 200, 175 to 225, 175 to 250, 175 to 275, 175 to 300, 175 to 325, 175 to 350, 175 to 375, 175 to 400, 175 to 425, 175 to 450, 175 to 475, 175 to 500, 200 to 250, 200 to 275, 200 to 300, 200 to 325, 200 to 350, 200 to 375, 200 to 400, 200 to 425, 200 to 450, 200 to 475, 200 to 500, 225 to 250, 225 to 275, 225 to 300, 225 to 325, 225 to 350, 225 to 375, 225 to 400, 225 to 425, 225 to 450, 225 to 475, 225 to 500, 250 to 275, 250 to 300, 275 to 300, 275 to 325, 275 to 350, 275 to 375, 275 to 400, 275 to 425, 275 to 450, 275 to 475, 275 to 500, 300 to 325, 300 to 350, 300 to 375, 300 to 400, 300 to 425, 300 to 450, 300 to 475, 300 to 500, 325 to 350, 325 to 375, 325 to 400, 325 to 425, 325 to 450, 325 to 475, 325 to 500, 350 to 375, 350 to 400, 350 to 425, 350 to 450, 350 to 475, 350 to 500, 375 to 400, 375 to 425, 375 to 450, 375 to 475, 375 to 500, 400 to 425, 400 to 450, 400 to 475, 400 to 500, 425 to 450, 425 to 475, 425 to 500, 450 to 475, 450 to 500, or 475 to 500 nucleotides in length. In some embodiments, the region of complementarity or substantial complementarity of the first editing template to the target double stranded DNA sequence is about 10 nucleotides in length. In some embodiments, the region of complementarity or substantial complementarity of the first editing template to the target double stranded DNA sequence is about 15 nucleotides in length. In some embodiments, the region of complementarity or substantial complementarity of the first editing template to the target double stranded DNA sequence is about 20 nucleotides in length. In some embodiments, the region of complementarity or substantial complementarity of the first editing template to the target double stranded DNA sequence is about 21, 22, 23, 24, or 25 nucleotides in length.

In some embodiments, the region of complementarity or substantial complementarity of the second editing template to the target double stranded DNA sequence is about 5 to 10, 5 to 15, 5 to 20, 5 to 25, 5 to 30, 5 to 35, 5 to 40, 5 to 45, 5 to 50, 5 to 55, 5 to 60, 5 to 65, 5 to 70, 5 to 75, 5 to 80, 5 to 85, 5 to 90, 5 to 95, 5 to 100, 5 to 110, 5 to 120, 5 to 130, 5 to 140, 5 to 150, 5 to 175, 5 to 200, 5 to 225, 5 to 250, 5 to 275, 5 to 300, 5 to 325, 5 to 350, 5 to 375, 5 to 400, 5 to 425, 5 to 450, 5 to 475, 5 to 500, 10 to 15, 10 to 20, 10 to 25, 10 to 30, 10 to 35, 10 to 40, 10 to 45, 10 to 50, 10 to 55, 10 to 60, 10 to 65, 10 to 70, 10 to 75, 10 to 80, 10 to 85, 10 to 90, 10 to 95, 10 to 100, 10 to 110, 10 to 120, 10 to 130, 10 to 140, 10 to 150, 10 to 175, 10 to 200, 10 to 225, 10 to 250, 10 to 275, 10 to 300, 10 to 325, 10 to 350, 10 to 375, 10 to 400, 10 to 425, 10 to 450, 10 to 475, 10 to 500, 15 to 20, 15 to 25, 15 to 30, 15 to 35, 15 to 40, 15 to 45, 15 to 50, 15 to 55, 15 to 60, 15 to 65, 15 to 70, 15 to 75, 15 to 80, 15 to 85, 15 to 90, 15 to 95, 15 to 100, 15 to 110, 15 to 120, 15 to 130, 15 to 140, 15 to 150, 15 to 175, 15 to 200, 15 to 225, 15 to 250, 15 to 275, 15 to 300, 15 to 325, 15 to 350, 15 to 375, 15 to 400, 15 to 425, 15 to 450, 15 to 475, 15 to 500, 20 to 25, 20 to 30, 20 to 35, 20 to 40, 20 to 45, 20 to 50, 20 to 55, 20 to 60, 20 to 65, 20 to 70, 20 to 75, 20 to 80, 20 to 85, 20 to 90, 20 to 95, 20 to 100, 20 to 110, 20 to 120, 20 to 130, 20 to 140, 20 to 150, 20 to 175, 20 to 200, 20 to 225, 20 to 250, 20 to 275, 20 to 300, 20 to 325, 20 to 350, 20 to 375, 20 to 400, 20 to 425, 20 to 450, 20 to 475, 20 to 500, 30 to 35, 30 to 40, 30 to 45, 30 to 50, 30 to 55, 30 to 60, 30 to 65, 30 to 70, 30 to 75, 30 to 80, 30 to 85, 30 to 90, 30 to 95, 30 to 100, 30 to 110, 30 to 120, 30 to 130, 30 to 140, 30 to 150, 30 to 175, 30 to 200, 30 to 225, 30 to 250, 30 to 275, 30 to 300, 30 to 325, 30 to 350, 30 to 375, 30 to 400, 30 to 425, 30 to 450, 30 to 475, 30 to 500, 40 to 45, 40 to 50, 40 to 55, 40 to 60, 40 to 65, 40 to 70, 40 to 75, 40 to 80, 40 to 85, 40 to 90, 40 to 95, 40 to 100, 40 to 110, 40 to 120, 40 to 130, 40 to 140, 40 to 150, 40 to 175, 40 to 200, 40 to 225, 40 to 250, 40 to 275, 40 to 300, 40 to 325, 40 to 350, 40 to 375, 40 to 400, 40 to 425, 40 to 450, 40 to 475, 40 to 500, 50 to 55, 50 to 60, 50 to 65, 50 to 70, 50 to 75, 50 to 80, 50 to 85, 50 to 90, 50 to 95, 50 to 100, 50 to 110, 50 to 120, 50 to 130, 50 to 140, 50 to 150, 50 to 175, 50 to 200, 50 to 225, 50 to 250, 50 to 275, 50 to 300, 50 to 325, 50 to 350, 50 to 375, 50 to 400, 50 to 425, 50 to 450, 50 to 475, 50 to 500, 75 to 80, 75 to 85, 75 to 90, 75 to 95, 75 to 100, 75 to 110, 75 to 120, 75 to 130, 75 to 140, 75 to 150, 75 to 175, 75 to 200, 75 to 225, 75 to 250, 75 to 275, 75 to 300, 75 to 325, 75 to 350, 75 to 375, 75 to 400, 75 to 425, 75 to 450, 75 to 475, 75 to 500, 100 to 110, 100 to 120, 100 to 130, 100 to 140, 100 to 150, 100 to 175, 100 to 200, 100 to 225, 100 to 250, 100 to 275, 100 to 300, 100 to 325, 100 to 350, 100 to 375, 100 to 400, 100 to 425, 100 to 450, 100 to 475, 100 to 500, 125 to 150, 125 to 175, 125 to 200, 125 to 225, 125 to 250, 125 to 275, 125 to 300, 125 to 325, 125 to 350, 125 to 375, 125 to 400, 125 to 425, 125 to 450, 125 to 475, 125 to 500, 150 to 175, 150 to 200, 150 to 225, 150 to 250, 150 to 275, 150 to 300, 150 to 325, 150 to 350, 150 to 375, 150 to 400, 150 to 425, 150 to 450, 150 to 475, 150 to 500, 175 to 200, 175 to 225, 175 to 250, 175 to 275, 175 to 300, 175 to 325, 175 to 350, 175 to 375, 175 to 400, 175 to 425, 175 to 450, 175 to 475, 175 to 500, 200 to 250, 200 to 275, 200 to 300, 200 to 325, 200 to 350, 200 to 375, 200 to 400, 200 to 425, 200 to 450, 200 to 475, 200 to 500, 225 to 250, 225 to 275, 225 to 300, 225 to 325, 225 to 350, 225 to 375, 225 to 400, 225 to 425, 225 to 450, 225 to 475, 225 to 500, 250 to 275, 250 to 300, 275 to 300, 275 to 325, 275 to 350, 275 to 375, 275 to 400, 275 to 425, 275 to 450, 275 to 475, 275 to 500, 300 to 325, 300 to 350, 300 to 375, 300 to 400, 300 to 425, 300 to 450, 300 to 475, 300 to 500, 325 to 350, 325 to 375, 325 to 400, 325 to 425, 325 to 450, 325 to 475, 325 to 500, 350 to 375, 350 to 400, 350 to 425, 350 to 450, 350 to 475, 350 to 500, 375 to 400, 375 to 425, 375 to 450, 375 to 475, 375 to 500, 400 to 425, 400 to 450, 400 to 475, 400 to 500, 425 to 450, 425 to 475, 425 to 500, 450 to 475, 450 to 500, or 475 to 500 nucleotides in length. In some embodiments, the region of complementarity or substantial complementarity of the second editing template to the target double stranded DNA sequence is about 10 nucleotides in length. In some embodiments, the region of complementarity or substantial complementarity of the second editing template to the target double stranded DNA sequence is about 15 nucleotides in length. In some embodiments, the region of complementarity or substantial complementarity of the second editing template to the target double stranded DNA sequence is about 20 nucleotides in length. In some embodiments, the region of complementarity or substantial complementarity of the second editing template to the target double stranded DNA sequence is about 21, 22, 23, 24, or 25 nucleotides in length.

In some embodiments, the first editing template comprises a region that does not have complementarity or substantial complementarity (the non-complementarity region) to the second editing template. In some embodiments, the first editing template comprises a region of complementarity or substantial complementarity to the second editing template, and further comprises a non-complementarity region to the second editing template. In some embodiments, the first editing template comprises a region of complementarity or substantial complementarity to the second editing template, a non-complementarity region to the second editing template, and a region of complementarity or substantial complementarity to the target double stranded DNA sequence.

In some embodiments, the second editing template comprises a region that does not have complementarity or substantial complementarity (the non-complementarity region) to the first editing template. In some embodiments, the second editing template comprises a region of complementarity or substantial complementarity to the first editing template, and further comprises a non-complementarity region to the first editing template. In some embodiments, the second editing template comprises a region of complementarity or substantial complementarity to the first editing template, a non-complementarity region to the first editing template, and a region of complementarity or substantial complementarity to the target double stranded DNA sequence.

In some embodiments, the region of non-complementarity of the first editing template to the second editing template and the region of non-complementarity of the second editing template to the first editing template are of the same length. In some embodiments, the region of non-complementarity of the first editing template to the second editing template and the region of non-complementarity of the second editing template to the first editing template are of different lengths. In some embodiments, the first editing template and the second editing template both comprise a region of non-complementarity to each other. In some embodiments, the first editing template comprises a region of non-complementarity to the second editing template, and the second editing template does not comprise a region of non-complementarity to the first editing template. In some embodiments, the second editing template comprises a region of non-complementarity to the first editing template, and the first editing template does not comprise a region of non-complementarity to the second editing template. For example, the first editing template may be complementary or substantially complementary to the second editing template through its entire length, while the second editing template comprises a region that does not have complementarity to the first editing template, or vice versa.

In some embodiments, the region of non-complementarity of the first editing template to the second editing template is about 5 to 10, 5 to 15, 5 to 20, 5 to 25, 5 to 30, 5 to 35, 5 to 40, 5 to 45, 5 to 50, 5 to 55, 5 to 60, 5 to 65, 5 to 70, 5 to 75, 5 to 80, 5 to 85, 5 to 90, 5 to 95, 5 to 100, 5 to 110, 5 to 120, 5 to 130, 5 to 140, 5 to 150, 5 to 175, 5 to 200, 5 to 225, 5 to 250, 5 to 275, 5 to 300, 5 to 325, 5 to 350, 5 to 375, 5 to 400, 5 to 425, 5 to 450, 5 to 475, 5 to 500, 10 to 15, 10 to 20, 10 to 25, 10 to 30, 10 to 35, 10 to 40, 10 to 45, 10 to 50, 10 to 55, 10 to 60, 10 to 65, 10 to 70, 10 to 75, 10 to 80, 10 to 85, 10 to 90, 10 to 95, 10 to 100, 10 to 110, 10 to 120, 10 to 130, 10 to 140, 10 to 150, 10 to 175, 10 to 200, 10 to 225, 10 to 250, 10 to 275, 10 to 300, 10 to 325, 10 to 350, 10 to 375, 10 to 400, 10 to 425, 10 to 450, 10 to 475, 10 to 500, 15 to 20, 15 to 25, 15 to 30, 15 to 35, 15 to 40, 15 to 45, 15 to 50, 15 to 55, 15 to 60, 15 to 65, 15 to 70, 15 to 75, 15 to 80, 15 to 85, 15 to 90, 15 to 95, 15 to 100, 15 to 110, 15 to 120, 15 to 130, 15 to 140, 15 to 150, 15 to 175, 15 to 200, 15 to 225, 15 to 250, 15 to 275, 15 to 300, 15 to 325, 15 to 350, 15 to 375, 15 to 400, 15 to 425, 15 to 450, 15 to 475, 15 to 500, 20 to 25, 20 to 30, 20 to 35, 20 to 40, 20 to 45, 20 to 50, 20 to 55, 20 to 60, 20 to 65, 20 to 70, 20 to 75, 20 to 80, 20 to 85, 20 to 90, 20 to 95, 20 to 100, 20 to 110, 20 to 120, 20 to 130, 20 to 140, 20 to 150, 20 to 175, 20 to 200, 20 to 225, 20 to 250, 20 to 275, 20 to 300, 20 to 325, 20 to 350, 20 to 375, 20 to 400, 20 to 425, 20 to 450, 20 to 475, 20 to 500, 30 to 35, 30 to 40, 30 to 45, 30 to 50, 30 to 55, 30 to 60, 30 to 65, 30 to 70, 30 to 75, 30 to 80, 30 to 85, 30 to 90, 30 to 95, 30 to 100, 30 to 110, 30 to 120, 30 to 130, 30 to 140, 30 to 150, 30 to 175, 30 to 200, 30 to 225, 30 to 250, 30 to 275, 30 to 300, 30 to 325, 30 to 350, 30 to 375, 30 to 400, 30 to 425, 30 to 450, 30 to 475, 30 to 500, 40 to 45, 40 to 50, 40 to 55, 40 to 60, 40 to 65, 40 to 70, 40 to 75, 40 to 80, 40 to 85, 40 to 90, 40 to 95, 40 to 100, 40 to 110, 40 to 120, 40 to 130, 40 to 140, 40 to 150, 40 to 175, 40 to 200, 40 to 225, 40 to 250, 40 to 275, 40 to 300, 40 to 325, 40 to 350, 40 to 375, 40 to 400, 40 to 425, 40 to 450, 40 to 475, 40 to 500, 50 to 55, 50 to 60, 50 to 65, 50 to 70, 50 to 75, 50 to 80, 50 to 85, 50 to 90, 50 to 95, 50 to 100, 50 to 110, 50 to 120, 50 to 130, 50 to 140, 50 to 150, 50 to 175, 50 to 200, 50 to 225, 50 to 250, 50 to 275, 50 to 300, 50 to 325, 50 to 350, 50 to 375, 50 to 400, 50 to 425, 50 to 450, 50 to 475, 50 to 500, 75 to 80, 75 to 85, 75 to 90, 75 to 95, 75 to 100, 75 to 110, 75 to 120, 75 to 130, 75 to 140, 75 to 150, 75 to 175, 75 to 200, 75 to 225, 75 to 250, 75 to 275, 75 to 300, 75 to 325, 75 to 350, 75 to 375, 75 to 400, 75 to 425, 75 to 450, 75 to 475, 75 to 500, 100 to 110, 100 to 120, 100 to 130, 100 to 140, 100 to 150, 100 to 175, 100 to 200, 100 to 225, 100 to 250, 100 to 275, 100 to 300, 100 to 325, 100 to 350, 100 to 375, 100 to 400, 100 to 425, 100 to 450, 100 to 475, 100 to 500, 125 to 150, 125 to 175, 125 to 200, 125 to 225, 125 to 250, 125 to 275, 125 to 300, 125 to 325, 125 to 350, 125 to 375, 125 to 400, 125 to 425, 125 to 450, 125 to 475, 125 to 500, 150 to 175, 150 to 200, 150 to 225, 150 to 250, 150 to 275, 150 to 300, 150 to 325, 150 to 350, 150 to 375, 150 to 400, 150 to 425, 150 to 450, 150 to 475, 150 to 500, 175 to 200, 175 to 225, 175 to 250, 175 to 275, 175 to 300, 175 to 325, 175 to 350, 175 to 375, 175 to 400, 175 to 425, 175 to 450, 175 to 475, 175 to 500, 200 to 250, 200 to 275, 200 to 300, 200 to 325, 200 to 350, 200 to 375, 200 to 400, 200 to 425, 200 to 450, 200 to 475, 200 to 500, 225 to 250, 225 to 275, 225 to 300, 225 to 325, 225 to 350, 225 to 375, 225 to 400, 225 to 425, 225 to 450, 225 to 475, 225 to 500, 250 to 275, 250 to 300, 275 to 300, 275 to 325, 275 to 350, 275 to 375, 275 to 400, 275 to 425, 275 to 450, 275 to 475, 275 to 500, 300 to 325, 300 to 350, 300 to 375, 300 to 400, 300 to 425, 300 to 450, 300 to 475, 300 to 500, 325 to 350, 325 to 375, 325 to 400, 325 to 425, 325 to 450, 325 to 475, 325 to 500, 350 to 375, 350 to 400, 350 to 425, 350 to 450, 350 to 475, 350 to 500, 375 to 400, 375 to 425, 375 to 450, 375 to 475, 375 to 500, 400 to 425, 400 to 450, 400 to 475, 400 to 500, 425 to 450, 425 to 475, 425 to 500, 450 to 475, 450 to 500, or 475 to 500 nucleotides in length. In some embodiments, the region of non-complementarity of the first editing template to the second editing template is about 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 or more nucleotides in length.

In some embodiments, the region of non-complementarity of the second editing template to the first editing template is about 5 to 10, 5 to 15, 5 to 20, 5 to 25, 5 to 30, 5 to 35, 5 to 40, 5 to 45, 5 to 50, 5 to 55, 5 to 60, 5 to 65, 5 to 70, 5 to 75, 5 to 80, 5 to 85, 5 to 90, 5 to 95, 5 to 100, 5 to 110, 5 to 120, 5 to 130, 5 to 140, 5 to 150, 5 to 175, 5 to 200, 5 to 225, 5 to 250, 5 to 275, 5 to 300, 5 to 325, 5 to 350, 5 to 375, 5 to 400, 5 to 425, 5 to 450, 5 to 475, 5 to 500, 10 to 15, 10 to 20, 10 to 25, 10 to 30, 10 to 35, 10 to 40, 10 to 45, 10 to 50, 10 to 55, 10 to 60, 10 to 65, 10 to 70, 10 to 75, 10 to 80, 10 to 85, 10 to 90, 10 to 95, 10 to 100, 10 to 110, 10 to 120, 10 to 130, 10 to 140, 10 to 150, 10 to 175, 10 to 200, 10 to 225, 10 to 250, 10 to 275, 10 to 300, 10 to 325, 10 to 350, 10 to 375, 10 to 400, 10 to 425, 10 to 450, 10 to 475, 10 to 500, 15 to 20, 15 to 25, 15 to 30, 15 to 35, 15 to 40, 15 to 45, 15 to 50, 15 to 55, 15 to 60, 15 to 65, 15 to 70, 15 to 75, 15 to 80, 15 to 85, 15 to 90, 15 to 95, 15 to 100, 15 to 110, 15 to 120, 15 to 130, 15 to 140, 15 to 150, 15 to 175, 15 to 200, 15 to 225, 15 to 250, 15 to 275, 15 to 300, 15 to 325, 15 to 350, 15 to 375, 15 to 400, 15 to 425, 15 to 450, 15 to 475, 15 to 500, 20 to 25, 20 to 30, 20 to 35, 20 to 40, 20 to 45, 20 to 50, 20 to 55, 20 to 60, 20 to 65, 20 to 70, 20 to 75, 20 to 80, 20 to 85, 20 to 90, 20 to 95, 20 to 100, 20 to 110, 20 to 120, 20 to 130, 20 to 140, 20 to 150, 20 to 175, 20 to 200, 20 to 225, 20 to 250, 20 to 275, 20 to 300, 20 to 325, 20 to 350, 20 to 375, 20 to 400, 20 to 425, 20 to 450, 20 to 475, 20 to 500, 30 to 35, 30 to 40, 30 to 45, 30 to 50, 30 to 55, 30 to 60, 30 to 65, 30 to 70, 30 to 75, 30 to 80, 30 to 85, 30 to 90, 30 to 95, 30 to 100, 30 to 110, 30 to 120, 30 to 130, 30 to 140, 30 to 150, 30 to 175, 30 to 200, 30 to 225, 30 to 250, 30 to 275, 30 to 300, 30 to 325, 30 to 350, 30 to 375, 30 to 400, 30 to 425, 30 to 450, 30 to 475, 30 to 500, 40 to 45, 40 to 50, 40 to 55, 40 to 60, 40 to 65, 40 to 70, 40 to 75, 40 to 80, 40 to 85, 40 to 90, 40 to 95, 40 to 100, 40 to 110, 40 to 120, 40 to 130, 40 to 140, 40 to 150, 40 to 175, 40 to 200, 40 to 225, 40 to 250, 40 to 275, 40 to 300, 40 to 325, 40 to 350, 40 to 375, 40 to 400, 40 to 425, 40 to 450, 40 to 475, 40 to 500, 50 to 55, 50 to 60, 50 to 65, 50 to 70, 50 to 75, 50 to 80, 50 to 85, 50 to 90, 50 to 95, 50 to 100, 50 to 110, 50 to 120, 50 to 130, 50 to 140, 50 to 150, 50 to 175, 50 to 200, 50 to 225, 50 to 250, 50 to 275, 50 to 300, 50 to 325, 50 to 350, 50 to 375, 50 to 400, 50 to 425, 50 to 450, 50 to 475, 50 to 500, 75 to 80, 75 to 85, 75 to 90, 75 to 95, 75 to 100, 75 to 110, 75 to 120, 75 to 130, 75 to 140, 75 to 150, 75 to 175, 75 to 200, 75 to 225, 75 to 250, 75 to 275, 75 to 300, 75 to 325, 75 to 350, 75 to 375, 75 to 400, 75 to 425, 75 to 450, 75 to 475, 75 to 500, 100 to 110, 100 to 120, 100 to 130, 100 to 140, 100 to 150, 100 to 175, 100 to 200, 100 to 225, 100 to 250, 100 to 275, 100 to 300, 100 to 325, 100 to 350, 100 to 375, 100 to 400, 100 to 425, 100 to 450, 100 to 475, 100 to 500, 125 to 150, 125 to 175, 125 to 200, 125 to 225, 125 to 250, 125 to 275, 125 to 300, 125 to 325, 125 to 350, 125 to 375, 125 to 400, 125 to 425, 125 to 450, 125 to 475, 125 to 500, 150 to 175, 150 to 200, 150 to 225, 150 to 250, 150 to 275, 150 to 300, 150 to 325, 150 to 350, 150 to 375, 150 to 400, 150 to 425, 150 to 450, 150 to 475, 150 to 500, 175 to 200, 175 to 225, 175 to 250, 175 to 275, 175 to 300, 175 to 325, 175 to 350, 175 to 375, 175 to 400, 175 to 425, 175 to 450, 175 to 475, 175 to 500, 200 to 250, 200 to 275, 200 to 300, 200 to 325, 200 to 350, 200 to 375, 200 to 400, 200 to 425, 200 to 450, 200 to 475, 200 to 500, 225 to 250, 225 to 275, 225 to 300, 225 to 325, 225 to 350, 225 to 375, 225 to 400, 225 to 425, 225 to 450, 225 to 475, 225 to 500, 250 to 275, 250 to 300, 275 to 300, 275 to 325, 275 to 350, 275 to 375, 275 to 400, 275 to 425, 275 to 450, 275 to 475, 275 to 500, 300 to 325, 300 to 350, 300 to 375, 300 to 400, 300 to 425, 300 to 450, 300 to 475, 300 to 500, 325 to 350, 325 to 375, 325 to 400, 325 to 425, 325 to 450, 325 to 475, 325 to 500, 350 to 375, 350 to 400, 350 to 425, 350 to 450, 350 to 475, 350 to 500, 375 to 400, 375 to 425, 375 to 450, 375 to 475, 375 to 500, 400 to 425, 400 to 450, 400 to 475, 400 to 500, 425 to 450, 425 to 475, 425 to 500, 450 to 475, 450 to 500, or 475 to 500 nucleotides in length. In some embodiments, the region of non-complementarity of the second editing template to the first editing template is about 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 or more nucleotides in length.

In some embodiments, the first PEgRNA and the second PEgRNA have the same gRNA cores. In some embodiments, the first PEgRNA and the second PEgRNA have different gRNA cores.

Exemplary combinations of PEgRNA components, e.g., spacer, PBS, and edit template/RTT, as well as combinations of each PEgRNA are provided in Tables 1A-36B. Tables 1A-19B contain PEgRNA and components of exemplary 5′ PEgRNAs. Tables 20A-36B contain PEgRNA and components of exemplary 3′ PEgRNAs. Each table corresponds to a specific spacer number. Each of these tables labeled with “A” contain four columns: from left to right, the first column is the sequence number, the second column provides the sequence of the component by reference to a SEQ ID NO. consistent with ST.26, the third column provides a description, and the fourth column provides the actual sequence. Each of these tables labeled with “B” include sequences of PEgRNAs and contain five columns: from left to right, the first column is the sequence number, the second column provides the sequence of the component by reference to a SEQ ID NO. consistent with ST.26, the third column provides the actual sequence, the fourth column provides the length of the PBS of the PEgRNA, and the fifth column provides a RTT Pairing number. Each 5′ PEgRNA having a specific RTT Pairing number can be used in a dual prime editing system with a 3′ PEgRNA that has the same RTT Pairing number. Although all the sequences provided in Tables 1A-36B are RNA sequences, “T” is used instead of a “U” in the sequences for consistency with the ST.26 standard used in the accompanying sequence listing.

Lengthy table referenced here
US20240360476A1-20241031-T00001
Please refer to the end of the specification for access instructions.

Lengthy table referenced here
US20240360476A1-20241031-T00002
Please refer to the end of the specification for access instructions.

Lengthy table referenced here
US20240360476A1-20241031-T00003
Please refer to the end of the specification for access instructions.

Lengthy table referenced here
US20240360476A1-20241031-T00004
Please refer to the end of the specification for access instructions.

Lengthy table referenced here
US20240360476A1-20241031-T00005
Please refer to the end of the specification for access instructions.

Lengthy table referenced here
US20240360476A1-20241031-T00006
Please refer to the end of the specification for access instructions.

Lengthy table referenced here
US20240360476A1-20241031-T00007
Please refer to the end of the specification for access instructions.

Lengthy table referenced here
US20240360476A1-20241031-T00008
Please refer to the end of the specification for access instructions.

Lengthy table referenced here
US20240360476A1-20241031-T00009
Please refer to the end of the specification for access instructions.

Lengthy table referenced here
US20240360476A1-20241031-T00010
Please refer to the end of the specification for access instructions.

Lengthy table referenced here
US20240360476A1-20241031-T00011
Please refer to the end of the specification for access instructions.

Lengthy table referenced here
US20240360476A1-20241031-T00012
Please refer to the end of the specification for access instructions.

Lengthy table referenced here
US20240360476A1-20241031-T00013
Please refer to the end of the specification for access instructions.

Lengthy table referenced here
US20240360476A1-20241031-T00014
Please refer to the end of the specification for access instructions.

Lengthy table referenced here
US20240360476A1-20241031-T00015
Please refer to the end of the specification for access instructions.

Lengthy table referenced here
US20240360476A1-20241031-T00016
Please refer to the end of the specification for access instructions.

Lengthy table referenced here
US20240360476A1-20241031-T00017
Please refer to the end of the specification for access instructions.

Lengthy table referenced here
US20240360476A1-20241031-T00018
Please refer to the end of the specification for access instructions.

Lengthy table referenced here
US20240360476A1-20241031-T00019
Please refer to the end of the specification for access instructions.

Lengthy table referenced here
US20240360476A1-20241031-T00020
Please refer to the end of the specification for access instructions.

Lengthy table referenced here
US20240360476A1-20241031-T00021
Please refer to the end of the specification for access instructions.

Lengthy table referenced here
US20240360476A1-20241031-T00022
Please refer to the end of the specification for access instructions.

Lengthy table referenced here
US20240360476A1-20241031-T00023
Please refer to the end of the specification for access instructions.

Lengthy table referenced here
US20240360476A1-20241031-T00024
Please refer to the end of the specification for access instructions.

Lengthy table referenced here
US20240360476A1-20241031-T00025
Please refer to the end of the specification for access instructions.

Lengthy table referenced here
US20240360476A1-20241031-T00026
Please refer to the end of the specification for access instructions.

Lengthy table referenced here
US20240360476A1-20241031-T00027
Please refer to the end of the specification for access instructions.

Lengthy table referenced here
US20240360476A1-20241031-T00028
Please refer to the end of the specification for access instructions.

Lengthy table referenced here
US20240360476A1-20241031-T00029
Please refer to the end of the specification for access instructions.

Lengthy table referenced here
US20240360476A1-20241031-T00030
Please refer to the end of the specification for access instructions.

Lengthy table referenced here
US20240360476A1-20241031-T00031
Please refer to the end of the specification for access instructions.

Lengthy table referenced here
US20240360476A1-20241031-T00032
Please refer to the end of the specification for access instructions.

Lengthy table referenced here
US20240360476A1-20241031-T00033
Please refer to the end of the specification for access instructions.

Lengthy table referenced here
US20240360476A1-20241031-T00034
Please refer to the end of the specification for access instructions.

Lengthy table referenced here
US20240360476A1-20241031-T00035
Please refer to the end of the specification for access instructions.

Lengthy table referenced here
US20240360476A1-20241031-T00036
Please refer to the end of the specification for access instructions.

Lengthy table referenced here
US20240360476A1-20241031-T00037
Please refer to the end of the specification for access instructions.

Lengthy table referenced here
US20240360476A1-20241031-T00038
Please refer to the end of the specification for access instructions.

Lengthy table referenced here
US20240360476A1-20241031-T00039
Please refer to the end of the specification for access instructions.

Lengthy table referenced here
US20240360476A1-20241031-T00040
Please refer to the end of the specification for access instructions.

Lengthy table referenced here
US20240360476A1-20241031-T00041
Please refer to the end of the specification for access instructions.

Lengthy table referenced here
US20240360476A1-20241031-T00042
Please refer to the end of the specification for access instructions.

Lengthy table referenced here
US20240360476A1-20241031-T00043
Please refer to the end of the specification for access instructions.

Lengthy table referenced here
US20240360476A1-20241031-T00044
Please refer to the end of the specification for access instructions.

Lengthy table referenced here
US20240360476A1-20241031-T00045
Please refer to the end of the specification for access instructions.

Lengthy table referenced here
US20240360476A1-20241031-T00046
Please refer to the end of the specification for access instructions.

Lengthy table referenced here
US20240360476A1-20241031-T00047
Please refer to the end of the specification for access instructions.

Lengthy table referenced here
US20240360476A1-20241031-T00048
Please refer to the end of the specification for access instructions.

Lengthy table referenced here
US20240360476A1-20241031-T00049
Please refer to the end of the specification for access instructions.

Lengthy table referenced here
US20240360476A1-20241031-T00050
Please refer to the end of the specification for access instructions.

Lengthy table referenced here
US20240360476A1-20241031-T00051
Please refer to the end of the specification for access instructions.

Lengthy table referenced here
US20240360476A1-20241031-T00052
Please refer to the end of the specification for access instructions.

Lengthy table referenced here
US20240360476A1-20241031-T00053
Please refer to the end of the specification for access instructions.

Lengthy table referenced here
US20240360476A1-20241031-T00054
Please refer to the end of the specification for access instructions.

Lengthy table referenced here
US20240360476A1-20241031-T00055
Please refer to the end of the specification for access instructions.

Lengthy table referenced here
US20240360476A1-20241031-T00056
Please refer to the end of the specification for access instructions.

Lengthy table referenced here
US20240360476A1-20241031-T00057
Please refer to the end of the specification for access instructions.

Lengthy table referenced here
US20240360476A1-20241031-T00058
Please refer to the end of the specification for access instructions.

Lengthy table referenced here
US20240360476A1-20241031-T00059
Please refer to the end of the specification for access instructions.

Lengthy table referenced here
US20240360476A1-20241031-T00060
Please refer to the end of the specification for access instructions.

Lengthy table referenced here
US20240360476A1-20241031-T00061
Please refer to the end of the specification for access instructions.

Lengthy table referenced here
US20240360476A1-20241031-T00062
Please refer to the end of the specification for access instructions.

Lengthy table referenced here
US20240360476A1-20241031-T00063
Please refer to the end of the specification for access instructions.

Lengthy table referenced here
US20240360476A1-20241031-T00064
Please refer to the end of the specification for access instructions.

Lengthy table referenced here
US20240360476A1-20241031-T00065
Please refer to the end of the specification for access instructions.

Lengthy table referenced here
US20240360476A1-20241031-T00066
Please refer to the end of the specification for access instructions.

Lengthy table referenced here
US20240360476A1-20241031-T00067
Please refer to the end of the specification for access instructions.

Lengthy table referenced here
US20240360476A1-20241031-T00068
Please refer to the end of the specification for access instructions.

Lengthy table referenced here
US20240360476A1-20241031-T00069
Please refer to the end of the specification for access instructions.

Lengthy table referenced here
US20240360476A1-20241031-T00070
Please refer to the end of the specification for access instructions.

Lengthy table referenced here
US20240360476A1-20241031-T00071
Please refer to the end of the specification for access instructions.

Lengthy table referenced here
US20240360476A1-20241031-T00072
Please refer to the end of the specification for access instructions.

### Tables 5A and 5B

Tables 5A and 5B provide exemplary 5′ PEgRNAs and components that can be used with any prime editor containing a Cas9 protein capable of recognizing a NGG PAM sequence, wherein N is A, G, C, or T. The PEgRNAs exemplified in Tables 5A and 5B comprise: (a) a spacer comprising at its 3′ end a sequence corresponding to nucleotides 5-20 of sequence number 77, (b) a gRNA core capable of binding to a Cas9 protein, and (c) an extension arm comprising (i) an editing template and (ii) a primer binding site (PBS) comprising at its 5′ end the sequence corresponding to sequence number 92.

The PEgRNA spacer can be, for example, 16-22 nucleotides in length. In some embodiments, the PEgNA spacer is 16-20 nucleotides in length. In some embodiments, the PEgRNA spacer comprises at its 3′ end nucleotides 5-20, 4-20, 3-20, 2-20, or 1-20 of sequence number 77. In some embodiments, the PEgRNA spacer comprises the sequence of sequence number 77. In some embodiments, the PEgRNA spacer is 20 nucleotides in length. Any of these spacers exemplified can be referred to as 5′ spacer No. 5.

The PBS can be, for example, 3 to 19 nucleotides in length. In some embodiments, the PBS is 3 to 17 nucleotides in length and corresponds to sequence number 78, 79, 80, 81, 8 [symbol] , 84, 85, 86, 87, 88, 89, 90, 91, or 92. In some cases, a PBS length of no more than 3 nucleot [symbol] less than the PEgRNA spacer length is chosen.

The 5′ PEgRNAs of Tables 5A and 5B can be used in a dual prime editing system or composition further comprising any suitable 3′ PEgRNA. The prime editing system can be used, e.g., for editing a DMPK gene harboring 50 or more CTG repeats.

In some embodiments, a prime editing composition comprises a 5′ PEgRNA having sequences or components exemplified in Tables 5A and 5B and a 3′ PEgRNA, wherein the editing templates of the 5′ PEgRNA and the 3′ PEgRNA each comprises a region of complementarity to each other. For example, the editing template of the 5′ PEgRNA can comprise any of the RTT sequences provided in Table 37. The terms “editing template” and “RTT” may be used interchangeably. Each of the RTT sequences in Table 37 is assigned a RTT Pairing number that reflects the complementarity relationship between a 5′ RTT and a 3′ RTT. The 5′ PEgRNA can be used in a dual prime editing system that further comprises a 3′ PEgRNA, e.g., a 3′ PEgRNA having a spacer sequence and a corresponding PBS as exemplified in Tables 20A-36B, wherein the 3′ PEgRNA comprises an editing template comprising a RTT sequence having a region of complementarity to the 5′ RTT. The 3′ RTT can comprise any of the RTT sequences provided in Table 38, wherein the 3′ RTT sequence has the same RTT Pairing number as that of the 5′ RTT. The editing templates of the 5′ PEgRNA and the 3′ PEgRNA can be completely complementary to each other, wherein the 5′ editing template and the 3′ editing template have the same length and perfect complementarity to each other. Alternatively, the 5′ editing template can comprise a region of complementarity to the 3′ editing template, and further comprise a non-complementary region to the 3′ editing template. The 3′ editing template can comprise a region of complementarity to the 5′ editing template, and further comprise a non-complementary region to the 5′ editing template. The non-complementary region can be at the 5′ end or the 3′ end of the editing templates. Contacting the target DMPK gene with the prime editing system can result in deletion of the sequence between the 5′ PEgRNA nick and the 3′ PEgRNA nick (the IND) and insertion of the region of complementarity between the 5′ editing template and the 3′ editing template (the OD), or insertion of the region of complementarity between the 5′ editing template and the 3′ editing template as well as the non-complementarity regions (together referred to as the RD), and hence replace the CTG repeats with the sequence of the OD or the RD. In some embodiments, the 5′ PEgRNA comprises a sequence selected from sequence numbers 93-468 and 3495. In some embodiments, the 5′ PEgRNA comprises a sequence selected from sequence numbers 93-95. In some embodiments, the 5′ PEgRNA comprises a sequence selected from sequence numbers 96-180 and 3495. In some embodiments, the 5′ PEgRNA comprises a sequence selected from sequence numbers 181-436. In some embodiments, the 5′ PEgRNA comprises a sequence selected from sequence numbers 436-468. Each of the 5′ PEgRNA is assigned a RTT Pairing number, and can be used in a dual prime editing composition with a 3′ PEgRNA comprising an RTT having the same RTT Pairing number.

In some embodiments, a prime editing composition comprises a 5′ PEgRNA or component thereof exemplified in Tables 5A and 5B and a 3′ PEgRNA, wherein the editing template of the 5′ PEgRNA comprises a region of identity to the spacer of the 3′ PEgRNA. In some embodiments, the editing template of the 3′ PEgRNA comprises a region of identity to the spacer of the 5′ PEgRNA. In some embodiments, the 5′ editing template comprises a region of complementarity to a region on the second strand of the DMPK gene that is directly 3′ to nucleotide 3 of the search target sequence of the 3′ PEgRNA. In some embodiments, the 3′ editing template comprises a region of complementarity to a region on the first strand of the DMPK gene that is directly 3′ to nucleotide 3 of the search target sequence of the 5′ PEgRNA. For example, the extension arm of the 5′ PEgRNA can comprise an editing template comprising the sequence of any of the RTT sequences provided in Table 39. Each of the RTT sequences in Table 39 is assigned a Pairing 3′ spacer number. The 5′ PEgRNA can be used in a dual prime editing system with a 3′ PEgRNA, wherein the 3′ PEgRNA comprises a spacer that has the Pairing 3′ spacer number assigned to the 5′ RTT in Table 39. Contacting the target DMPK gene with the prime editing system can result in deletion of the sequence between the 5′ PEgRNA nick and the 3′ PEgRNA nick (the IND) without insertion of any exogenous sequence (a “clean deletion”). For example, the 3′ PEgRNA can also comprises a “clean deletion” RTT, e.g., a 3′ RTT selected from Table 40 that is assigned a Pairing 5′ spacer number 5. Alternatively, the 5′ editing template and the 3′ editing template can further comprise a region of complementarity to each other, and the region of complementarity can replace the IND in the DMPK gene. In some embodiments, the 5′ PEgRNA is part of a dual prime editing composition that further comprises a 3′ PEgRNA, wherein the 5′ PEgRNA comprises a RTT comprising a sequence selected from sequence numbers 3459-3461 and 3467-3471, and the 3′ PEgRNA comprises a spacer comprising nucleotides 5-20 of sequence number 1525. In some embodiments, the 5′ PEgRNA is part of a dual prime editing composition that further comprises a 3′ PEgRNA, wherein the 5′ PEgRNA comprises a RTT comprising a sequence selected from sequence numbers 3462-3466, and the 3′ PEgRNA comprises a spacer comprising nucleotides 5-20 of sequence number 2353. In some embodiments, the 5′ PEgRNA is part of a dual prime editing composition that further comprises a 3′ PEgRNA, wherein the 5′ PEgRNA comprises a RTT comprising a sequence selected from sequence numbers 3472-3476, and the 3′ PEgRNA comprises a spacer comprising nucleotides 5-20 of sequence number 1936. In some embodiments, the 5′ PEgRNA is part of a dual prime editing composition that further comprises a 3′ PEgRNA, wherein the 5′ PEgRNA comprises a RTT comprising nucleotides x-40 of sequence number 3647, 3648, 3649, 3650, 3651, 3652, 3653, 3654, 3655, 3656, 3657, 3658, 3659, 3660, 3661, 3662, or 3663, wherein the 3′ PEgRNA comprises a spacer comprising nucleotides 5-20 of sequence number 1900, 1918, 1403, 1936, 2263, 2353, 1503, 2281, 2673, 1525, 2299, 2317, 1864, 1359, 2335, 1882, or 1381 respectively in the same order, and wherein x is any integer from 1 to 31. In some embodiments, the 5′ PEgRNA comprises a sequence selected from sequence numbers 469-483 and 3683-3697. Each 5′ PEgRNA is assigned a RTT Pairing number, which corresponds to a Pairing 3′ spacer number in Table 39. The 5′ PEgRNA can be used in a dual prime editing system or composition with a 3′ PEgRNA comprising a spacer of the same Pairing 3′ spacer number. In some embodiments, the 5′ PEgRNA comprises a sequence selected from sequence numbers 473, 475, 476, 479, and 480, and is used in a prime editing system further comprising a 3′ PEgRNA having a spacer comprising nucleotides 5-20, 4-20, 3-20, 2-20, or 1-20 of SEQ ID NO: 2353. In some embodiments, the 5′ PEgRNA comprises a sequence selected from sequence numbers 469, 470, 481, 482, and 483, and is used in a prime editing system further comprising a 3′ PEgRNA having a spacer comprising nucleotides 5-20, 4-20, 3-20, 2-20, or 1-20 of SEQ ID NO: 1525. In some embodiments, the 5′ PEgRNA comprises a sequence selected from sequence numbers 471, 472, 474, 477, 478, and 3688-3692, and is used in a prime editing system further comprising a 3′ PEgRNA having a spacer comprising nucleotides 5-20, 4-20, 3-20, 2-20, or 1-20 of SEQ ID NO: 1936. In some embodiments, the 5′ PEgRNA comprises a sequence selected from sequence numbers 3683-3687, and is used in a prime editing system further comprising a 3′ PEgRNA having a spacer comprising nucleotides 5-20, 4-20, 3-20, 2-20, or 1-20 of SEQ ID NO: 1900. In some embodiments, the 5′ PEgRNA comprises a sequence selected from sequence numbers 3693-3697, and is used in a prime editing system further comprising a 3′ PEgRNA having a spacer comprising nucleotides 5-20, 4-20, 3-20, 2-20, or 1-20 of SEQ ID No. 2673.

### Tables 8A and 8B

Tables 8A and 8B provide exemplary 5′ PEgRNAs and components that can be used with any prime editor containing a Cas9 protein capable of recognizing a NGG PAM sequence, wherein N is A, G, C, or T. The PEgRNAs exemplified in Tables 8A and 8B comprise: (a) a spacer comprising at its 3′ end a sequence corresponding to nucleotides 5-20 of sequence number 536, (b) a gRNA core capable of binding to a Cas9 protein, and (c) an extension arm comprising (i) an editing template and (ii) a primer binding site (PBS) comprising at its 5′ end the sequence corresponding to sequence number 551.

The PEgRNA spacer can be, for example, 16-22 nucleotides in length. In some embodiments, the PEgNA spacer is 16-20 nucleotides in length. In some embodiments, the PEgRNA spacer comprises at its 3′ end nucleotides 5-20, 4-20, 3-20, 2-20, or 1-20 of sequence number 536. In some embodiments, the PEgRNA spacer comprises the sequence of sequence number 536. In some embodiments, the PEgRNA spacer is 20 nucleotides in length. Any of these spacers exemplified can be referred to as 5′ spacer No. 8.

The PBS can be, for example, 3 to 19 nucleotides in length. In some embodiments, the PBS is 3 to 17 nucleotides in length and corresponds to sequence number 537, 538, 539, 540, 541, 542, 543, 544, 545, 546, 547, 548, 549, 550, or 551. In some cases, a PBS length of no more than 3 nucleotides less than the PEgRNA spacer length is chosen.

The 5′ PEgRNAs of Tables 8A and 8B can be used in a dual prime editing system or composition further comprising any suitable 3′ PEgRNA. The prime editing system can be used, e.g., for editing a DMPK gene harboring 50 or more CTG repeats.

In some embodiments, a prime editing composition comprises a 5′ PEgRNA having sequences or components exemplified in Tables 8A and 8B and a 3′ PEgRNA, wherein the editing templates of the 5′ PEgRNA and the 3′ PEgRNA each comprises a region of complementarity to each other. For example, the editing template of the 5′ PEgRNA can comprise any of the RTT sequences provided in Table 37. The terms “editing template” and “RTT” may be used interchangeably. Each of the RTT sequences in Table 37 is assigned a RTT Pairing number that reflects the complementarity relationship between a 5′ RTT and a 3′ RTT. The 5′ PEgRNA can be used in a dual prime editing system that further comprises a 3′ PEgRNA, e.g., a 3′ PEgRNA having a spacer sequence and a corresponding PBS as exemplified in Tables 20A-36B, wherein the 3′ PEgRNA comprises an editing template comprising a RTT sequence having a region of complementarity to the 5′ RTT. The 3′ RTT can comprise any of the RTT sequences provided in Table 38, wherein the 3′ RTT sequence has the same RTT Pairing number as that of the 5′ RTT. The editing templates of the 5′ PEgRNA and the 3′ PEgRNA can be completely complementary to each other, wherein the 5′ editing template and the 3′ editing template have the same length and perfect complementarity to each other. Alternatively, the 5′ editing template can comprise a region of complementarity to the 3′ editing template, and further comprise a non-complementary region to the 3′ editing template. The 3′ editing template can comprise a region of complementarity to the 5′ editing template, and further comprise a non-complementary region to the 5′ editing template. The non-complementary region can be at the 5′ end or the 3′ end of the editing templates. Contacting the target DMPK gene with the prime editing system can result in deletion of the sequence between the 5′ PEgRNA nick and the 3′ PEgRNA nick (the IND) and insertion of the region of complementarity between the 5′ editing template and the 3′ editing template (the OD), or insertion of the region of complementarity between the 5′ editing template and the 3′ editing template as well as the non-complementarity regions (together referred to as the RD), and hence replace the CTG repeats with the sequence of the OD or the RD. In some embodiments, the 5′ PEgRNA comprises the sequence of sequence number 552 or 553. Each of the 5′ PEgRNA is assigned a RTT Pairing number, and can be used in a dual prime editing composition with a 3′ PEgRNA comprising an RTT having the same RTT Pairing number.

In some embodiments, a prime editing composition comprises a 5′ PEgRNA or component thereof exemplified in Tables 8A and 8B and a 3′ PEgRNA, wherein the editing template of the 5′ PEgRNA comprises a region of identity to the spacer of the 3′ PEgRNA. In some embodiments, the editing template of the 3′ PEgRNA comprises a region of identity to the spacer of the 5′ PEgRNA. In some embodiments, the 5′ editing template comprises a region of complementarity to a region on the second strand of the DMPK gene that is directly 3′ to nucleotide 3 of the search target sequence of the 3′ PEgRNA. In some embodiments, the 3′ editing template comprises a region of complementarity to a region on the first strand of the DMPK gene that is directly 3′ to nucleotide 3 of the search target sequence of the 5′ PEgRNA. For example, the extension arm of the 5′ PEgRNA can comprise an editing template comprising the sequence of any of the RTT sequences provided in Table 39. Each of the RTT sequences in Table 39 is assigned a Pairing 3′ spacer number. The 5′ PEgRNA can be used in a dual prime editing system with a 3′ PEgRNA, wherein the 3′ PEgRNA comprises a spacer that has the Pairing 3′ spacer number assigned to the 5′ RTT in Table 39. Contacting the target DMPK gene with the prime editing system can result in deletion of the sequence between the 5′ PEgRNA nick and the 3′ PEgRNA nick (the IND) without insertion of any exogenous sequence (a “clean deletion”). For example, the 3′ PEgRNA can also comprises a “clean deletion” RTT, e.g., a 3′ RTT selected from Table 40 that is assigned a Pairing 5′ spacer number 8. Alternatively, the 5′ editing template and the 3′ editing template can further comprise a region of complementarity to each other, and the region of complementarity can replace the IND in the DMPK gene. In some embodiments, the 5′ PEgRNA is part of a dual prime editing composition that further comprises a 3′ PEgRNA, wherein the 5′ PEgRNA comprises a RTT comprising a sequence selected from sequence numbers 3459-3461 and 3467-3471, and the 3′ PEgRNA comprises a spacer comprising nucleotides 5-20 of sequence number 1525. In some embodiments, the 5′ PEgRNA is part of a dual prime editing composition that further comprises a 3′ PEgRNA, wherein the 5′ PEgRNA comprises a RTT comprising a sequence selected from sequence numbers 3462-3466, and the 3′ PEgRNA comprises a spacer comprising nucleotides 5-20 of sequence number 2353. In some embodiments, the 5′ PEgRNA is part of a dual prime editing composition that further comprises a 3′ PEgRNA, wherein the 5′ PEgRNA comprises a RTT comprising a sequence selected from sequence numbers 3472-3476, and the 3′ PEgRNA comprises a spacer comprising nucleotides 5-20 of sequence number 1936. In some embodiments, the 5′ PEgRNA is part of a dual prime editing composition that further comprises a 3′ PEgRNA, wherein the 5′ PEgRNA comprises a RTT comprising nucleotides x-40 of sequence number 3647, 3648, 3649, 3650, 3651, 3652, 3653, 3654, 3655, 3656, 3657, 3658, 3659, 3660, 3661, 3662, or 3663, wherein the 3′ PEgRNA comprises a spacer comprising nucleotides 5-20 of sequence number 1900, 1918, 1403, 1936, 2263, 2353, 1503, 2281, 2673, 1525, 2299, 2317, 1864, 1359, 2335, 1882, or 1381 respectively in the same order, and wherein x is any integer from 1 to 31. In some embodiments, the 5′ PEgRNA comprises a sequence selected from sequence numbers 3698-3712. Each 5′ PEgRNA is assigned a RTT Pairing number, which corresponds to a Pairing 3′ spacer number in Table 39. The 5′ PEgRNA can be used in a dual prime editing system or composition with a 3′ PEgRNA comprising a spacer of the same Pairing 3′ spacer number. In some embodiments, the 5′ PEgRNA comprises a sequence selected from sequence numbers 3703-3707, and is used in a prime editing system further comprising a 3′ PEgRNA having a spacer comprising nucleotides 5-20, 4-20, 3-20, 2-20, or 1-20 of SEQ ID NO: 1936. In some embodiments, the 5′ PEgRNA comprises a sequence selected from sequence numbers 3698-3702, and is used in a prime editing system further comprising a 3′ PEgRNA having a spacer comprising nucleotides 5-20, 4-20, 3-20, 2-20, or 1-20 of SEQ ID NO: 1900. In some embodiments, the 5′ PEgRNA comprises a sequence selected from sequence numbers 3708-3712, and is used in a prime editing system further comprising a 3′ PEgRNA having a spacer comprising nucleotides 5-20, 4-20, 3-20, 2-20, or 1-20 of SEQ ID NO: 2673.

### Tables 18A and 18B

Tables 18A and 18B provide exemplary 5′ PEgRNAs and components that can be used with any prime editor containing a Cas9 protein capable of recognizing a NGG PAM sequence, wherein N is A, G, C, or T. The PEgRNAs exemplified in Tables 18A and 18B comprise: (a) a spacer comprising at its 3′ end a sequence corresponding to nucleotides 5-20 of sequence number 1019, (b) a gRNA core capable of binding to a Cas9 protein, and (c) an extension arm comprising (i) an editing template and (ii) a primer binding site (PBS) comprising at its 5′ end the sequence corresponding to sequence number 1034.

The PEgRNA spacer can be, for example, 16-22 nucleotides in length. In some embodiments, the PEgNA spacer is 16-20 nucleotides in length. In some embodiments, the PEgRNA spacer comprises at its 3′ end nucleotides 5-20, 4-20, 3-20, 2-20, or 1-20 of sequence number 1019. In some embodiments, the PEgRNA spacer comprises the sequence of sequence number 1019. In some embodiments, the PEgRNA spacer is 20 nucleotides in length. Any of these spacers exemplified can be referred to as 5′ spacer No. 18.

The PBS can be, for example, 3 to 19 nucleotides in length. In some embodiments, the PBS is 3 to 17 nucleotides in length and corresponds to sequence number 1020, 1021, 1022, 1023, 1024, 1025, 1026, 1027, 1028, 1029, 1030, 1031, 1032, 1033, or 1034. In some cases, a PBS length of no more than 3 nucleotides less than the PEgRNA spacer length is chosen.

The 5′ PEgRNAs of Tables 18A and 18B can be used in a dual prime editing system or composition further comprising any suitable 3′ PEgRNA. The prime editing system can be used, e.g., for editing a DMPK gene harboring 50 or more CTG repeats.

In some embodiments, a prime editing composition comprises a 5′ PEgRNA having sequences or components exemplified in Tables 18A and 18B and a 3′ PEgRNA, wherein the editing templates of the 5′ PEgRNA and the 3′ PEgRNA each comprises a region of complementarity to each other. For example, the editing template of the 5′ PEgRNA can comprise any of the RTT sequences provided in Table 37. The terms “editing template” and “RTT” may be used interchangeably. Each of the RTT sequences in Table 37 is assigned a RTT Pairing number that reflects the complementarity relationship between a 5′ RTT and a 3′ RTT. The 5′ PEgRNA can be used in a dual prime editing system that further comprises a 3′ PEgRNA, e.g., a 3′ PEgRNA having a spacer sequence and a corresponding PBS as exemplified in Tables 20A-36B, wherein the 3′ PEgRNA comprises an editing template comprising a RTT sequence having a region of complementarity to the 5′ RTT. The 3′ RTT can comprise any of the RTT sequences provided in Table 38, wherein the 3′ RTT sequence has the same RTT Pairing number as that of the 5′ RTT. The editing templates of the 5′ PEgRNA and the 3′ PEgRNA can be completely complementary to each other, wherein the 5′ editing template and the 3′ editing template have the same length and perfect complementarity to each other. Alternatively, the 5′ editing template can comprise a region of complementarity to the 3′ editing template, and further comprise a non-complementary region to the 3′ editing template. The 3′ editing template can comprise a region of complementarity to the 5′ editing template, and further comprise a non-complementary region to the 5′ editing template. The non-complementary region can be at the 5′ end or the 3′ end of the editing templates. Contacting the target DMPK gene with the prime editing system can result in deletion of the sequence between the 5′ PEgRNA nick and the 3′ PEgRNA nick (the IND) and insertion of the region of complementarity between the 5′ editing template and the 3′ editing template (the OD), or insertion of the region of complementarity between the 5′ editing template and the 3′ editing template as well as the non-complementarity regions (together referred to as the RD), and hence replace the CTG repeats with the sequence of the OD or the RD. In some embodiments, the 5′ PEgRNA comprises a sequence selected from sequence numbers 1035-1325. In some embodiments, the 5′ PEgRNA comprises the sequence of sequence number 1035 or 1036. In some embodiments, the 5′ PEgRNA comprises a sequence selected from sequence numbers 1037-1291. In some embodiments, the 5′ PEgRNA comprises a sequence selected from sequence numbers 1292-1325. Each of the 5′ PEgRNA is assigned a RTT Pairing number, and can be used in a dual prime editing composition with a 3′ PEgRNA comprising an RTT having the same RTT Pairing number.

In some embodiments, a prime editing composition comprises a 5′ PEgRNA or component thereof exemplified in Tables 18A and 18B and a 3′ PEgRNA, wherein the editing template of the 5′ PEgRNA comprises a region of identity to the spacer of the 3′ PEgRNA. In some embodiments, the editing template of the 3′ PEgRNA comprises a region of identity to the spacer of the 5′ PEgRNA. In some embodiments, the 5′ editing template comprises a region of complementarity to a region on the second strand of the DMPK gene that is directly 3′ to nucleotide 3 of the search target sequence of the 3′ PEgRNA. In some embodiments, the 3′ editing template comprises a region of complementarity to a region on the first strand of the DMPK gene that is directly 3′ to nucleotide 3 of the search target sequence of the 5′ PEgRNA. For example, the extension arm of the 5′ PEgRNA can comprise an editing template comprising the sequence of any of the RTT sequences provided in Table 39. Each of the RTT sequences in Table 39 is assigned a Pairing 3′ spacer number. The 5′ PEgRNA can be used in a dual prime editing system with a 3′ PEgRNA, wherein the 3′ PEgRNA comprises a spacer that has the Pairing 3′ spacer number assigned to the 5′ RTT in Table 39. Contacting the target DMPK gene with the prime editing system can result in deletion of the sequence between the 5′ PEgRNA nick and the 3′ PEgRNA nick (the IND) without insertion of any exogenous sequence (a “clean deletion”). For example, the 3′ PEgRNA can also comprises a “clean deletion” RTT, e.g., a 3′ RTT selected from Table 40 that is assigned a Pairing 5′ spacer number 18. Alternatively, the 5′ editing template and the 3′ editing template can further comprise a region of complementarity to each other, and the region of complementarity can replace the IND in the DMPK gene. In some embodiments, the 5′ PEgRNA is part of a dual prime editing composition that further comprises a 3′ PEgRNA, wherein the 5′ PEgRNA comprises a RTT comprising a sequence selected from sequence numbers 3459-3461 and 3467-3471, and the 3′ PEgRNA comprises a spacer comprising nucleotides 5-20 of sequence number 1525. In some embodiments, the 5′ PEgRNA is part of a dual prime editing composition that further comprises a 3′ PEgRNA, wherein the 5′ PEgRNA comprises a RTT comprising a sequence selected from sequence numbers 3462-3466, and the 3′ PEgRNA comprises a spacer comprising nucleotides 5-20 of sequence number 2353. In some embodiments, the 5′ PEgRNA is part of a dual prime editing composition that further comprises a 3′ PEgRNA, wherein the 5′ PEgRNA comprises a RTT comprising a sequence selected from sequence numbers 3472-3476, and the 3′ PEgRNA comprises a spacer comprising nucleotides 5-20 of sequence number 1936. In some embodiments, the 5′ PEgRNA is part of a dual prime editing composition that further comprises a 3′ PEgRNA, wherein the 5′ PEgRNA comprises a RTT comprising nucleotides x-40 of sequence number 3647, 3648, 3649, 3650, 3651, 3652, 3653, 3654, 3655, 3656, 3657, 3658, 3659, 3660, 3661, 3662, or 3663, wherein the 3′ PEgRNA comprises a spacer comprising nucleotides 5-20 of sequence number 1900, 1918, 1403, 1936, 2263, 2353, 1503, 2281, 2673, 1525, 2299, 2317, 1864, 1359, 2335, 1882, or 1381 respectively in the same order, and wherein x is any integer from 1 to 31. In some embodiments, the 5′ PEgRNA comprises a sequence selected from sequence numbers 1326-1340 and 3713-3727. Each 5′ PEgRNA is assigned a RTT Pairing number, which corresponds to a Pairing 3′ spacer number in Table 39. The 5′ PEgRNA can be used in a dual prime editing system or composition with a 3′ PEgRNA comprising a spacer of the same Pairing 3′ spacer number. In some embodiments, the 5′ PEgRNA comprises a sequence selected from sequence numbers 1331-1335, and is used in a prime editing system further comprising a 3′ PEgRNA having a spacer comprising nucleotides 5-20, 4-20, 3-20, 2-20, or 1-20 of SEQ ID NO: 2353. In some embodiments, the 5′ PEgRNA comprises a sequence selected from sequence numbers 1336-1340, and is used in a prime editing system further comprising a 3′ PEgRNA having a spacer comprising nucleotides 5-20, 4-20, 3-20, 2-20, or 1-20 of SEQ ID NO: 1525. In some embodiments, the 5′ PEgRNA comprises a sequence selected from sequence numbers 1326-1330 and 3718-3722, and is used in a prime editing system further comprising a 3′ PEgRNA having a spacer comprising nucleotides 5-20, 4-20, 3-20, 2-20, or 1-20 of SEQ ID NO: 1936. In some embodiments, the 5′ PEgRNA comprises a sequence selected from sequence numbers 3713-3717, and is used in a prime editing system further comprising a 3′ PEgRNA having a spacer comprising nucleotides 5-20, 4-20, 3-20, 2-20, or 1-20 of SEQ ID NO: 1900. In some embodiments, the 5′ PEgRNA comprises a sequence selected from sequence numbers 3723-3727, and is used in a prime editing system further comprising a 3′ PEgRNA having a spacer comprising nucleotides 5-20, 4-20, 3-20, 2-20, or 1-20 of SEQ ID NO: 2673.

### Tables 27A and 27B

Tables 27A and 27B provide exemplary 3′ PEgRNAs and components that can be used with any prime editor containing a Cas9 protein capable of recognizing a NGG PAM sequence, wherein N is A, G, C, or T. The PEgRNAs exemplified in Tables 27A and 27B comprise: (a) a spacer comprising at its 3′ end a sequence corresponding to nucleotides 5-20 of sequence number 1900, (b) a gRNA core capable of binding to a Cas9 protein, and (c) an extension arm comprising (i) an editing template and (ii) a primer binding site (PBS) comprising at its 5′ end the sequence corresponding to sequence number 1915.

The PEgRNA spacer can be, for example, 16-22 nucleotides in length. In some embodiments, the PEgNA spacer is 16-20 nucleotides in length. In some embodiments, the PEgRNA spacer comprises at its 3′ end nucleotides 5-20, 4-20, 3-20, 2-20, or 1-20 of sequence number 1900. In some embodiments, the PEgRNA spacer comprises the sequence of sequence number 1900. In some embodiments, the PEgRNA spacer is 20 nucleotides in length. Any of these spacers exemplified can be referred to as 3′ spacer No. 8.

The PBS can be, for example, 3 to 19 nucleotides in length. In some embodiments, the PBS is 3 to 17 nucleotides in length and corresponds to sequence number 1901, 1902, 1903, 1904, 1905, 1906, 1907, 1908, 1909, 1910, 1911, 1912, 1913, 1914, or 1915. In some cases, a PBS length of no more than 3 nucleotides less than the PEgRNA spacer length is chosen.

The 3′ PEgRNAs of Tables 27A and 27B can be used in a dual prime editing system or composition further comprising any suitable 5′ PEgRNA. The prime editing system can be used, e.g., for editing a DMPK gene harboring 50 or more CTG repeats.

In some embodiments, a prime editing composition comprises a 3′ PEgRNA having sequences or components exemplified in Tables 27A and 27B and a 5′ PEgRNA, wherein the editing templates of the 3′ PEgRNA and the 5′ PEgRNA each comprises a region of complementarity to each other. For example, the editing template of the 3′ PEgRNA can comprise any of the RTT sequences provided in Table 38. The terms “editing template” and “RTT” may be used interchangeably. Each of the RTT sequences in Table 38 is assigned a RTT Pairing number that reflects the complementarity relationship between a 5′ RTT and a 3′ RTT. The 3′ PEgRNA can be used in a dual prime editing system that further comprises a 5′ PEgRNA, e.g., a 5′ PEgRNA having a spacer sequence and a corresponding PBS as exemplified in Tables 1A-19B, wherein the 5′ PEgRNA comprises an editing template comprising a RTT sequence having a region of complementarity to the 3′ RTT. The 5′ RTT can comprise any of the RTT sequences provided in Table 37, wherein the 5′ RTT sequence has the same RTT Pairing number as that of the 3′ RTT. The editing templates of the 5′ PEgRNA and the 3′ PEgRNA can be completely complementary to each other, wherein the 5′ editing template and the 3′ editing template have the same length and perfect complementarity to each other. Alternatively, the 5′ editing template can comprise a region of complementarity to the 3′ editing template, and further comprise a non-complementary region to the 3′ editing template. The 3′ editing template can comprise a region of complementarity to the 5′ editing template, and further comprise a non-complementary region to the 5′ editing template. The non-complementary region can be at the 5′ end or the 3′ end of the editing templates. Contacting the target DMPK gene with the prime editing system can result in deletion of the sequence between the 5′ PEgRNA nick and the 3′ PEgRNA nick (the IND) and insertion of the region of complementarity between the 5′ editing template and the 3′ editing template (the OD), or insertion of the region of complementarity between the 5′ editing template and the 3′ editing template as well as the non-complementarity regions (together referred to as the RD), and hence replace the CTG repeats with the sequence of the OD or the RD. In some embodiments, the 3′ PEgRNA comprises the sequence of sequence number 1916 or 1917. Each of the 3′ PEgRNA is assigned a RTT Pairing number, and can be used in a dual prime editing composition with a 5′ PEgRNA comprising an RTT having the same RTT Pairing number.

In some embodiments, a prime editing composition comprises a 3′ PEgRNA or component thereof exemplified in Tables 27A and 27B and a 5′ PEgRNA, wherein the editing template of the 3′ PEgRNA comprises a region of identity to the spacer of the 5′ PEgRNA. In some embodiments, the editing template of the 5′ PEgRNA comprises a region of identity to the spacer of the 3′ PEgRNA. In some embodiments, the 3′ editing template comprises a region of complementarity to a region on the first strand of the DMPK gene that is directly 3′ to nucleotide 3 of the search target sequence of the 5′ PEgRNA. In some embodiments, the 5′ editing template comprises a region of complementarity to a region on the second strand of the DMPK gene that is directly 3′ to nucleotide 3 of the search target sequence of the 3′ PEgRNA. For example, the extension arm of the 3′ PEgRNA can comprise an editing template comprising the sequence of any of the RTT sequences provided in Table 40. Each of the RTT sequences in Table 40 is assigned a Pairing 5′ spacer number. The 3′ PEgRNA can be used in a dual prime editing system with a 5′ PEgRNA, wherein the 5′ PEgRNA comprises a spacer that has the Pairing 5′ spacer number assigned to the 3′ RTT in Table 40. Contacting the target DMPK gene with the prime editing system can result in deletion of the sequence between the 5′ PEgRNA nick and the 3′ PEgRNA nick (the IND) without insertion of any exogenous sequence (a “clean deletion”). For example, the 5′ PEgRNA can also comprises a “clean deletion” RTT, e.g., a 5′ RTT selected from Table 39 that is assigned a Pairing 3′ spacer number 8. Alternatively, the 5′ editing template and the 3′ editing template can further comprise a region of complementarity to each other, and the region of complementarity can replace the IND in the DMPK gene. In some embodiments, the 3′ PEgRNA is part of a dual prime editing composition that further comprises a 5′ PEgRNA, wherein the 3′ PEgRNA comprises a RTT comprising a sequence selected from sequence numbers 3477-3479, and the 5′ PEgRNA comprises a spacer comprising nucleotides 5-20 of sequence number 484. In some embodiments, the 3′ PEgRNA is part of a dual prime editing composition that further comprises a 5′ PEgRNA, wherein the 3′ PEgRNA comprises a RTT comprising a sequence selected from sequence numbers 3480-3484, and the 5′ PEgRNA comprises a spacer comprising nucleotides 5-20 of sequence number 77. In some embodiments, the 3′ PEgRNA is part of a dual prime editing composition that further comprises a 5′ PEgRNA, wherein the 3′ PEgRNA comprises a RTT comprising a sequence selected from sequence numbers 3485-3489, and the 5′ PEgRNA comprises a spacer comprising nucleotides 5-20 of sequence number 590. In some embodiments, the 3′ PEgRNA is part of a dual prime editing composition that further comprises a 5′ PEgRNA, wherein the 3′ PEgRNA comprises a RTT comprising a sequence selected from sequence numbers 3490-3494, and the 5′ PEgRNA comprises a spacer comprising nucleotides 5-20 of sequence number 1019. In some embodiments, the 3′ PEgRNA is part of a dual prime editing composition that further comprises a 5′ PEgRNA, wherein the 3′ PEgRNA comprises a RTT comprising nucleotides x-40 of sequence number 3664, 3665, 3666, 3667, 3668, 3669, 3670, 3671, 3672, 3673, 3674, 3675, 3676, 3677, 3678, 3679, 3680, 3681, or 3682, wherein the 5′ PEgRNA comprises a spacer comprising nucleotides 5-20 of sequence number 518, 536, 554, 572, 590, 39, 912, 929, 947, 965, 983, 1001, 1019, 1341, 20, 77, 484, 58, or 1 respectively in the same order, and wherein x is any integer from 1 to 31. In some embodiments, the 3′ PEgRNA comprises a sequence selected from sequence numbers 3728-3742. Each 3′ PEgRNA is assigned a RTT Pairing number, which corresponds to a Pairing 5′ spacer number in Table 40. The 3′ PEgRNA can be used in a dual prime editing system or composition with a 5′ PEgRNA comprising a spacer of the same Pairing 5′ spacer number. In some embodiments, the 3′ PEgRNA comprises a sequence selected from sequence numbers 3733-3737, and is used in a prime editing system further comprising a 5′ PEgRNA having a spacer comprising nucleotides 5-20, 4-20, 3-20, 2-20, or 1-20 of SEQ ID NO: 77. In some embodiments, the 3′ PEgRNA comprises a sequence selected from sequence numbers 3728-3732, and is used in a prime editing system further comprising a 5′ PEgRNA having a spacer comprising nucleotides 5-20, 4-20, 3-20, 2-20, or 1-20 of SEQ ID NO: 1019. In some embodiments, the 3′ PEgRNA comprises a sequence selected from sequence numbers 3738-3742, and is used in a prime editing system further comprising a 5′ PEgRNA having a spacer comprising nucleotides 5-20, 4-20, 3-20, 2-20, or 1-20 of SEQ ID NO: 536.

### Tables 29A and 29B

Tables 29A and 29B provide exemplary 3′ PEgRNAs and components that can be used with any prime editor containing a Cas9 protein capable of recognizing a NGG PAM sequence, wherein N is A, G, C, or T. The PEgRNAs exemplified in Tables 29A and 29B comprise: (a) a spacer comprising at its 3′ end a sequence corresponding to nucleotides 5-20 of sequence number 1936, (b) a gRNA core capable of binding to a Cas9 protein, and (c) an extension arm comprising (i) an editing template and (ii) a primer binding site (PBS) comprising at its 5′ end the sequence corresponding to sequence number 1951.

The PEgRNA spacer can be, for example, 16-22 nucleotides in length. In some embodiments, the PEgNA spacer is 16-20 nucleotides in length. In some embodiments, the PEgRNA spacer comprises at its 3′ end nucleotides 5-20, 4-20, 3-20, 2-20, or 1-20 of sequence number 1936. In some embodiments, the PEgRNA spacer comprises the sequence of sequence number 1936. In some embodiments, the PEgRNA spacer is 20 nucleotides in length. Any of these spacers exemplified can be referred to as 3′ spacer No. 10.

The PBS can be, for example, 3 to 19 nucleotides in length. In some embodiments, the PBS is 3 to 17 nucleotides in length and corresponds to sequence number 1937, 1938, 1939, 1940, 1941, 1942, 1943, 1944, 1945, 1946, 1947, 1948, 1949, 1950, or 1951. In some cases, a PBS length of no more than 3 nucleotides less than the PEgRNA spacer length is chosen.

The 3′ PEgRNAs of Tables 29A and 29B can be used in a dual prime editing system or composition further comprising any suitable 5′ PEgRNA. The prime editing system can be used, e.g., for editing a DMPK gene harboring 50 or more CTG repeats.

In some embodiments, a prime editing composition comprises a 3′ PEgRNA having sequences or components exemplified in Tables 29A and 29B and a 5′ PEgRNA, wherein the editing templates of the 3′ PEgRNA and the 5′ PEgRNA each comprises a region of complementarity to each other. For example, the editing template of the 3′ PEgRNA can comprise any of the RTT sequences provided in Table 38. The terms “editing template” and “RTT” may be used interchangeably. Each of the RTT sequences in Table 38 is assigned a RTT Pairing number that reflects the complementarity relationship between a 5′ RTT and a 3′ RTT. The 3′ PEgRNA can be used in a dual prime editing system that further comprises a 5′ PEgRNA, e.g., a 5′ PEgRNA having a spacer sequence and a corresponding PBS as exemplified in Tables 1A-19B, wherein the 5′ PEgRNA comprises an editing template comprising a RTT sequence having a region of complementarity to the 3′ RTT. The 5′ RTT can comprise any of the RTT sequences provided in Table 37, wherein the 5′ RTT sequence has the same RTT Pairing number as that of the 3′ RTT. The editing templates of the 5′ PEgRNA and the 3′ PEgRNA can be completely complementary to each other, wherein the 5′ editing template and the 3′ editing template have the same length and perfect complementarity to each other. Alternatively, the 5′ editing template can comprise a region of complementarity to the 3′ editing template, and further comprise a non-complementary region to the 3′ editing template. The 3′ editing template can comprise a region of complementarity to the 5′ editing template, and further comprise a non-complementary region to the 5′ editing template. The non-complementary region can be at the 5′ end or the 3′ end of the editing templates. Contacting the target DMPK gene with the prime editing system can result in deletion of the sequence between the 5′ PEgRNA nick and the 3′ PEgRNA nick (the IND) and insertion of the region of complementarity between the 5′ editing template and the 3′ editing template (the OD), or insertion of the region of complementarity between the 5′ editing template and the 3′ editing template as well as the non-complementarity regions (together referred to as the RD), and hence replace the CTG repeats with the sequence of the OD or the RD. In some embodiments, the 3′ PEgRNA comprises a sequence selected from sequence numbers 1952-2247. In some embodiments, the 3′ PEgRNA comprises a sequence selected from sequence numbers 1952-1961. In some embodiments, the 3′ PEgRNA comprises a sequence selected from sequence numbers 1962-2216. In some embodiments, the 3′ PEgRNA comprises a sequence selected from sequence numbers 2217-2247. Each of the 3′ PEgRNA is assigned a RTT Pairing number, and can be used in a dual prime editing composition with a 5′ PEgRNA comprising an RTT having the same RTT Pairing number.

In some embodiments, a prime editing composition comprises a 3′ PEgRNA or component thereof exemplified in Tables 29A and 29B and a 5′ PEgRNA, wherein the editing template of the 3′ PEgRNA comprises a region of identity to the spacer of the 5′ PEgRNA. In some embodiments, the editing template of the 5′ PEgRNA comprises a region of identity to the spacer of the 3′ PEgRNA. In some embodiments, the 3′ editing template comprises a region of complementarity to a region on the first strand of the DMPK gene that is directly 3′ to nucleotide 3 of the search target sequence of the 5′ PEgRNA. In some embodiments, the 5′ editing template comprises a region of complementarity to a region on the second strand of the DMPK gene that is directly 3′ to nucleotide 3 of the search target sequence of the 3′ PEgRNA. For example, the extension arm of the 3′ PEgRNA can comprise an editing template comprising the sequence of any of the RTT sequences provided in Table 40. Each of the RTT sequences in Table 40 is assigned a Pairing 5′ spacer number. The 3′ PEgRNA can be used in a dual prime editing system with a 5′ PEgRNA, wherein the 5′ PEgRNA comprises a spacer that has the Pairing 5′ spacer number assigned to the 3′ RTT in Table 40. Contacting the target DMPK gene with the prime editing system can result in deletion of the sequence between the 5′ PEgRNA nick and the 3′ PEgRNA nick (the IND) without insertion of any exogenous sequence (a “clean deletion”). For example, the 5′ PEgRNA can also comprises a “clean deletion” RTT, e.g., a 5′ RTT selected from Table 39 that is assigned a Pairing 3′ spacer number 10. Alternatively, the 5′ editing template and the 3′ editing template can further comprise a region of complementarity to each other, and the region of complementarity can replace the IND in the DMPK gene. In some embodiments, the 3′ PEgRNA is part of a dual prime editing composition that further comprises a 5′ PEgRNA, wherein the 3′ PEgRNA comprises a RTT comprising a sequence selected from sequence numbers 3477-3479, and the 5′ PEgRNA comprises a spacer comprising nucleotides 5-20 of sequence number 484. In some embodiments, the 3′ PEgRNA is part of a dual prime editing composition that further comprises a 5′ PEgRNA, wherein the 3′ PEgRNA comprises a RTT comprising a sequence selected from sequence numbers 3480-3484, and the 5′ PEgRNA comprises a spacer comprising nucleotides 5-20 of sequence number 77. In some embodiments, the 3′ PEgRNA is part of a dual prime editing composition that further comprises a 5′ PEgRNA, wherein the 3′ PEgRNA comprises a RTT comprising a sequence selected from sequence numbers 3485-3489, and the 5′ PEgRNA comprises a spacer comprising nucleotides 5-20 of sequence number 590. In some embodiments, the 3′ PEgRNA is part of a dual prime editing composition that further comprises a 5′ PEgRNA, wherein the 3′ PEgRNA comprises a RTT comprising a sequence selected from sequence numbers 3490-3494, and the 5′ PEgRNA comprises a spacer comprising nucleotides 5-20 of sequence number 1019. In some embodiments, the 3′ PEgRNA is part of a dual prime editing composition that further comprises a 5′ PEgRNA, wherein the 3′ PEgRNA comprises a RTT comprising nucleotides x-40 of sequence number 3664, 3665, 3666, 3667, 3668, 3669, 3670, 3671, 3672, 3673, 3674, 3675, 3676, 3677, 3678, 3679, 3680, 3681, or 3682, wherein the 5′ PEgRNA comprises a spacer comprising nucleotides 5-20 of sequence number 518, 536, 554, 572, 590, 39, 912, 929, 947, 965, 983, 1001, 1019, 1341, 20, 77, 484, 58, or 1 respectively in the same order, and wherein x is any integer from 1 to 31. In some embodiments, the 3′ PEgRNA comprises a sequence selected from sequence numbers 2248-2262 and 3743-3757. Each 3′ PEgRNA is assigned a RTT Pairing number, which corresponds to a Pairing 5′ spacer number in Table 40. The 3′ PEgRNA can be used in a dual prime editing system or composition with a 5′ PEgRNA comprising a spacer of the same Pairing 5′ spacer number. In some embodiments, the 3′ PEgRNA comprises a sequence selected from sequence numbers 2248-2252 and 3748-3752, and is used in a prime editing system further comprising a 5′ PEgRNA having a spacer comprising nucleotides 5-20, 4-20, 3-20, 2-20, or 1-20 of SEQ ID NO: 77. In some embodiments, the 3′ PEgRNA comprises a sequence selected from sequence numbers 2258-2262, and is used in a prime editing system further comprising a 5′ PEgRNA having a spacer comprising nucleotides 5-20, 4-20, 3-20, 2-20, or 1-20 of SEQ ID NO: 590. In some embodiments, the 3′ PEgRNA comprises a sequence selected from sequence numbers 2253-2257 and 3743-3747, and is used in a prime editing system further comprising a 5′ PEgRNA having a spacer comprising nucleotides 5-20, 4-20, 3-20, 2-20, or 1-20 of SEQ ID NO: 1019. In some embodiments, the 3′ PEgRNA comprises a sequence selected from sequence numbers 3753-3757, and is used in a prime editing system further comprising a 5′ PEgRNA having a spacer comprising nucleotides 5-20, 4-20, 3-20, 2-20, or 1-20 of SEQ ID NO: 536.

### Tables 36A and 36B

Tables 36A and 36B provide exemplary 3′ PEgRNAs and components that can be used with any prime editor containing a Cas9 protein capable of recognizing a NGG PAM sequence, wherein N is A, G, C, or T. The PEgRNAs exemplified in Tables 36A and 36B comprise: (a) a spacer comprising at its 3′ end a sequence corresponding to nucleotides 5-20 of sequence number 2673, (b) a gRNA core capable of binding to a Cas9 protein, and (c) an extension arm comprising (i) an editing template and (ii) a primer binding site (PBS) comprising at its 5′ end the sequence corresponding to sequence number 2688.

The PEgRNA spacer can be, for example, 16-22 nucleotides in length. In some embodiments, the PEgNA spacer is 16-20 nucleotides in length. In some embodiments, the PEgRNA spacer comprises at its 3′ end nucleotides 5-20, 4-20, 3-20, 2-20, or 1-20 of sequence number 2673. In some embodiments, the PEgRNA spacer comprises the sequence of sequence number 2673. In some embodiments, the PEgRNA spacer is 20 nucleotides in length. Any of these spacers exemplified can be referred to as 3′ spacer No. 17.

The PBS can be, for example, 3 to 19 nucleotides in length. In some embodiments, the PBS is 3 to 17 nucleotides in length and corresponds to sequence number 2674, 2675, 2676, 2677, 2678, 2679, 2680, 2681, 2682, 2683, 2684, 2685, 2686, 2687, or 2688. In some cases, a PBS length of no more than 3 nucleotides less than the PEgRNA spacer length is chosen.

The 3′ PEgRNAs of Tables 36A and 36B can be used in a dual prime editing system or composition further comprising any suitable 5′ PEgRNA. The prime editing system can be used, e.g., for editing a DMPK gene harboring 50 or more CTG repeats.

In some embodiments, a prime editing composition comprises a 3′ PEgRNA having sequences or components exemplified in Tables 36A and 36B and a 5′ PEgRNA, wherein the editing templates of the 3′ PEgRNA and the 5′ PEgRNA each comprises a region of complementarity to each other. For example, the editing template of the 3′ PEgRNA can comprise any of the RTT sequences provided in Table 38. The terms “editing template” and “RTT” may be used interchangeably. Each of the RTT sequences in Table 38 is assigned a RTT Pairing number that reflects the complementarity relationship between a 5′ RTT and a 3′ RTT. The 3′ PEgRNA can be used in a dual prime editing system that further comprises a 5′ PEgRNA, e.g., a 5′ PEgRNA having a spacer sequence and a corresponding PBS as exemplified in Tables 1A-19B, wherein the 5′ PEgRNA comprises an editing template comprising a RTT sequence having a region of complementarity to the 3′ RTT. The 5′ RTT can comprise any of the RTT sequences provided in Table 37, wherein the 5′ RTT sequence has the same RTT Pairing number as that of the 3′ RTT. The editing templates of the 5′ PEgRNA and the 3′ PEgRNA can be completely complementary to each other, wherein the 5′ editing template and the 3′ editing template have the same length and perfect complementarity to each other. Alternatively, the 5′ editing template can comprise a region of complementarity to the 3′ editing template, and further comprise a non-complementary region to the 3′ editing template. The 3′ editing template can comprise a region of complementarity to the 5′ editing template, and further comprise a non-complementary region to the 5′ editing template. The non-complementary region can be at the 5′ end or the 3′ end of the editing templates. Contacting the target DMPK gene with the prime editing system can result in deletion of the sequence between the 5′ PEgRNA nick and the 3′ PEgRNA nick (the IND) and insertion of the region of complementarity between the 5′ editing template and the 3′ editing template (the OD), or insertion of the region of complementarity between the 5′ editing template and the 3′ editing template as well as the non-complementarity regions (together referred to as the RD), and hence replace the CTG repeats with the sequence of the OD or the RD. In some embodiments, the 3′ PEgRNA comprises the sequence of sequence number 2689 or 2690. Each of the 3′ PEgRNA is assigned a RTT Pairing number, and can be used in a dual prime editing composition with a 5′ PEgRNA comprising an RTT having the same RTT Pairing number.

In some embodiments, a prime editing composition comprises a 3′ PEgRNA or component thereof exemplified in Tables 36A and 36B and a 5′ PEgRNA, wherein the editing template of the 3′ PEgRNA comprises a region of identity to the spacer of the 5′ PEgRNA. In some embodiments, the editing template of the 5′ PEgRNA comprises a region of identity to the spacer of the 3′ PEgRNA. In some embodiments, the 3′ editing template comprises a region of complementarity to a region on the first strand of the DMPK gene that is directly 3′ to nucleotide 3 of the search target sequence of the 5′ PEgRNA. In some embodiments, the 5′ editing template comprises a region of complementarity to a region on the second strand of the DMPK gene that is directly 3′ to nucleotide 3 of the search target sequence of the 3′ PEgRNA. For example, the extension arm of the 3′ PEgRNA can comprise an editing template comprising the sequence of any of the RTT sequences provided in Table 40. Each of the RTT sequences in Table 40 is assigned a Pairing 5′ spacer number. The 3′ PEgRNA can be used in a dual prime editing system with a 5′ PEgRNA, wherein the 5′ PEgRNA comprises a spacer that has the Pairing 5′ spacer number assigned to the 3′ RTT in Table 40. Contacting the target DMPK gene with the prime editing system can result in deletion of the sequence between the 5′ PEgRNA nick and the 3′ PEgRNA nick (the IND) without insertion of any exogenous sequence (a “clean deletion”). For example, the 5′ PEgRNA can also comprises a “clean deletion” RTT, e.g., a 5′ RTT selected from Table 39 that is assigned a Pairing 3′ spacer number 17. Alternatively, the 5′ editing template and the 3′ editing template can further comprise a region of complementarity to each other, and the region of complementarity can replace the IND in the DMPK gene. In some embodiments, the 3′ PEgRNA is part of a dual prime editing composition that further comprises a 5′ PEgRNA, wherein the 3′ PEgRNA comprises a RTT comprising a sequence selected from sequence numbers 3477-3479, and the 5′ PEgRNA comprises a spacer comprising nucleotides 5-20 of sequence number 484. In some embodiments, the 3′ PEgRNA is part of a dual prime editing composition that further comprises a 5′ PEgRNA, wherein the 3′ PEgRNA comprises a RTT comprising a sequence selected from sequence numbers 3480-3484, and the 5′ PEgRNA comprises a spacer comprising nucleotides 5-20 of sequence number 77. In some embodiments, the 3′ PEgRNA is part of a dual prime editing composition that further comprises a 5′ PEgRNA, wherein the 3′ PEgRNA comprises a RTT comprising a sequence selected from sequence numbers 3485-3489, and the 5′ PEgRNA comprises a spacer comprising nucleotides 5-20 of sequence number 590. In some embodiments, the 3′ PEgRNA is part of a dual prime editing composition that further comprises a 5′ PEgRNA, wherein the 3′ PEgRNA comprises a RTT comprising a sequence selected from sequence numbers 3490-3494, and the 5′ PEgRNA comprises a spacer comprising nucleotides 5-20 of sequence number 1019. In some embodiments, the 3′ PEgRNA is part of a dual prime editing composition that further comprises a 5′ PEgRNA, wherein the 3′ PEgRNA comprises a RTT comprising nucleotides x-40 of sequence number 3664, 3665, 3666, 3667, 3668, 3669, 3670, 3671, 3672, 3673, 3674, 3675, 3676, 3677, 3678, 3679, 3680, 3681, or 3682, wherein the 5′ PEgRNA comprises a spacer comprising nucleotides 5-20 of sequence number 518, 536, 554, 572, 590, 39, 912, 929, 947, 965, 983, 1001, 1019, 1341, 20, 77, 484, 58, or 1 respectively in the same order, and wherein x is any integer from 1 to 31. In some embodiments, the 3′ PEgRNA comprises a sequence selected from sequence numbers 3758-3772. Each 3′ PEgRNA is assigned a RTT Pairing number, which corresponds to a Pairing 5′ spacer number in Table 40. The 3′ PEgRNA can be used in a dual prime editing system or composition with a 5′ PEgRNA comprising a spacer of the same Pairing 5′ spacer number. In some embodiments, the 3′ PEgRNA comprises a sequence selected from sequence numbers 3763-3767, and is used in a prime editing system further comprising a 5′ PEgRNA having a spacer comprising nucleotides 5-20, 4-20, 3-20, 2-20, or 1-20 of SEQ ID NO: 77. In some embodiments, the 3′ PEgRNA comprises a sequence selected from sequence numbers 3758-3762, and is used in a prime editing system further comprising a 5′ PEgRNA having a spacer comprising nucleotides 5-20, 4-20, 3-20, 2-20, or 1-20 of SEQ ID NO: 1019. In some embodiments, the 3′ PEgRNA comprises a sequence selected from sequence numbers 3768-3772, and is used in a prime editing system further comprising a 5′ PEgRNA having a spacer comprising nucleotides 5-20, 4-20, 3-20, 2-20, or 1-20 of SEQ ID NO: 536.

### Tables 1A and 1B

Tables 1A and 1B provide exemplary 5′ PEgRNAs and components that can be used with any prime editor containing a Cas9 protein capable of recognizing a NGG PAM sequence, wherein N is A, G, C, or T. The PEgRNAs exemplified in Tables 1A and 1B comprise: (a) a spacer comprising at its 3′ end a sequence corresponding to nucleotides 5-20 of sequence number 1, (b) a gRNA core capable of binding to a Cas9 protein, and (c) an extension arm comprising (i) an editing template and (ii) a primer binding site (PBS) comprising at its 5′ end the sequence corresponding to sequence number 16.

The PEgRNA spacer can be, for example, 16-22 nucleotides in length. In some embodiments, the PEgNA spacer is 16-20 nucleotides in length. In some embodiments, the PEgRNA spacer comprises nucleotides 5-20, 4-20, 3-20, 2-20, or 1-20 of sequence number 1. In some embodiments, the PEgRNA spacer comprises the sequence of sequence number 1. In some embodiments, the PEgRNA spacer is 20 nucleotides in length. Any of these spacers exemplified can be referred to as 5′ spacer No. 1.

The PBS can be, for example, 3 to 19 nucleotides in length. In some embodiments, the PBS is 3 to 17 nucleotides in length and corresponds to a PBS sequence in Table 1A. In some cases, a PBS length of no more than 3 nucleotides less than the PEgRNA spacer length is chosen.

The 5′ PEgRNAs of Tables 1A and 1B can be used in a dual prime editing system or composition further comprising any suitable 3′ PEgRNA. The prime editing system can be used, e.g., for editing a DMPK gene harboring 50 or more CTG repeats in the DMPK gene.

In some embodiments, a prime editing composition comprises a 5′ PEgRNA or component thereof exemplified in Tables 1A and 1B and a 3′ PEgRNA, wherein the editing template of the 5′ PEgRNA and the 3′ PEgRNA each comprises a region of complementarity to each other. For example, the editing template of the 5′ PEgRNA can comprise any of the RTT sequences provided in Table 37. The terms editing template and RTT may be used interchangeably. Each of the RTT sequences in Table 37 is assigned a RTT pairing number that reflects the complementarity relationship between a 5′ RTT and a 3′ RTT. The 5′ PEgRNA can be used in a dual prime editing system that further comprises a 3′ PEgRNA, e.g., a 3′ PEgRNA having a spacer sequence and a corresponding PBS as exemplified in Tables 20A-36B, wherein the 3′ PEgRNA comprises an editing template comprising a RTT sequence having a region of complementarity to the 5′ RTT. The 3′ RTT can comprise any of the RTT sequences provided in Table 38, wherein the 3′ RTT sequence has the same RTT Pairing number as that of the 5′ RTT. The editing template of the 5′ PEgRNA and the 3′ PEgRNA can be completely complementary to each other, wherein the 5′ editing template and the 3′ editing template have the same length and perfect complementarity to each other. Alternatively, the 5′ editing template can comprise a region of complementarity to the 3′ editing template, and further comprise a non-complementary region to the 3′ editing template. The 3′ editing template can comprise a region of complementarity to the 5′ editing template, and further comprise a non-complementary region to the 5′ editing template. The non-complementary region can be at the 5′ end or the 3′ end of the editing templates. Contacting the target DMPK gene with the prime editing system can result in deletion of the sequence between the 5′ PEgRNA nick and the 3′ PEgRNA nick (the IND) and insertion of the region of complementarity between the 5′ editing template and the 3′ editing template (the OD), or insertion of the region of complementarity between the 5′ editing template and the 3′ editing template as well as the non-complementarity regions (the RD), and hence replace the CTG expansion with the sequence of the OD or the RD. Each of the 5′ PEgRNA is assigned a RTT pairing number, and can be used in a dual prime editing composition with a 3′ PEgRNA comprising an RTT having the same RTT pairing number.

In some embodiments, a prime editing composition comprises a 5′ PEgRNA or component thereof exemplified in Tables 1A and 1B and a 3′ PEgRNA, wherein the editing template of the 5′ PEgRNA comprises a region of identity to the spacer of the 3′ PEgRNA. In some embodiments, the editing template of the 3′ PEgRNA comprises a region of identity to the spacer of the 5′ PEgRNA. In some embodiments, the 5′ editing template comprises a region of complementarity to a region on the second strand of the DMPK gene that is directly 3′ to nucleotide 3 of the search target sequence of the 3′ PEgRNA. In some embodiments, the 3′ editing template comprises a region of complementarity to a region on the first strand of the DMPK gene that is directly 3′ to nucleotide 3 of the search target sequence of the 5′ PEgRNA. For example, the extension arm of the 5′ PEgRNA can comprise an editing template comprising the sequence of any of the RTT sequences provided in Table 39. Each of the RTT sequences in Table 39 is assigned a pairing 3′ spacer number. The 5′ PEgRNA can be used in a dual prime editing system with a 3′ PEgRNA, wherein the 3′ PEgRNA comprises a spacer that has the Pairing 3′ spacer number assigned to the 5′ RTT in Table 39. Contacting the target DMPK gene with the prime editing system can result in deletion of the sequence between the 5′ PEgRNA nick and the 3′ PEgRNA nick (the IND) without insertion of any exogenous sequence (a “clean deletion”). Alternatively, the 5′ editing template and the 3′ editing template can further comprise a region of complementarity to each other, and the region of complementarity can replace the IND in the DMPK gene. For example, in some embodiments, the 5′ PEgRNA is part of a dual prime editing composition that further comprises a 3′ PEgRNA, wherein the 5′ PEgRNA comprises a RTT comprising a sequence selected from sequence numbers 3459-3461 and 3467-3471, and the 3′ PEgRNA comprises a spacer comprising nucleotides 5-20 of sequence number 1525. In some embodiments, the 5′ PEgRNA is part of a dual prime editing composition that further comprises a 3′ PEgRNA, wherein the 5′ PEgRNA comprises a RTT comprising a sequence selected from sequence numbers 3462-3466, and the 3′ PEgRNA comprises a spacer comprising nucleotides 5-20 of sequence number 2353. In some embodiments, the 5′ PEgRNA is part of a dual prime editing composition that further comprises a 3′ PEgRNA, wherein the 5′ PEgRNA comprises a RTT comprising a sequence selected from sequence numbers 3472-3476, and the 3′ PEgRNA comprises a spacer comprising nucleotides 5-20 of sequence number 1936. In some embodiments, the 5′ PEgRNA is part of a dual prime editing composition that further comprises a 3′ PEgRNA, wherein the 5′ PEgRNA comprises a RTT comprising nucleotides x-40 of sequence number 3647, 3648, 3649, 3650, 3651, 3652, 3653, 3654, 3655, 3656, 3657, 3658, 3659, 3660, 3661, 3662, or 3663, wherein the 3′ PEgRNA comprises a spacer comprising nucleotides 5-20 of sequence number 1900, 1918, 1403, 1936, 2263, 2353, 1503, 2281, 2673, 1525, 2299, 2317, 1864, 1359, 2335, 1882, or 1381 respectively in the same order, and wherein x is any integer from 1 to 31. Preferably, the 3′ PEgRNA comprises a “clean deletion” RTT, for example, an RTT selected from Table 40 that has a Pairing 5′ spacer number that is 1. Each 5′ PEgRNA is assigned a RTT pairing number, which corresponds to a pairing 3′ spacer number in Table 39. The 5′ PEgRNA can be used in a dual prime editing system or composition with a 3′ PEgRNA comprising a spacer of the same pairing 3′ spacer number.

### Tables 2A and 2B

Tables 2A and 2B provide exemplary 5′ PEgRNAs and components that can be used with any prime editor containing a Cas9 protein capable of recognizing a NGG PAM sequence, wherein N is A, G, C, or T. The PEgRNAs exemplified in Tables 2A and 2B comprise: (a) a spacer comprising at its 3′ end a sequence corresponding to nucleotides 5-20 of sequence number 20, (b) a gRNA core capable of binding to a Cas9 protein, and (c) an extension arm comprising (i) an editing template and (ii) a primer binding site (PBS) comprising at its 5′ end the sequence corresponding to sequence number 35.

The PEgRNA spacer can be, for example, 16-22 nucleotides in length. In some embodiments, the PEgNA spacer is 16-20 nucleotides in length. In some embodiments, the PEgRNA spacer comprises nucleotides 5-20, 4-20, 3-20, 2-20, or 1-20 of sequence number 20. In some embodiments, the PEgRNA spacer comprises the sequence of sequence number 20. In some embodiments, the PEgRNA spacer is 20 nucleotides in length. Any of these spacers exemplified can be referred to as 5′ spacer No. 2.

The PBS can be, for example, 3 to 19 nucleotides in length. In some embodiments, the PBS is 3 to 17 nucleotides in length and corresponds to a PBS sequence in Table 2A. In some cases, a PBS length of no more than 3 nucleotides less than the PEgRNA spacer length is chosen.

The 5′ PEgRNAs of Tables 2A and 2B can be used in a dual prime editing system or composition further comprising any suitable 3′ PEgRNA. The prime editing system can be used, e.g., for editing a DMPK gene harboring 50 or more CTG repeats in the DMPK gene.

In some embodiments, a prime editing composition comprises a 5′ PEgRNA or component thereof exemplified in Tables 2A and 2B and a 3′ PEgRNA, wherein the editing template of the 5′ PEgRNA and the 3′ PEgRNA each comprises a region of complementarity to each other. For example, the editing template of the 5′ PEgRNA can comprise any of the RTT sequences provided in Table 37. The terms editing template and RTT may be used interchangeably. Each of the RTT sequences in Table 37 is assigned a RTT pairing number that reflects the complementarity relationship between a 5′ RTT and a 3′ RTT. The 5′ PEgRNA can be used in a dual prime editing system that further comprises a 3′ PEgRNA, e.g., a 3′ PEgRNA having a spacer sequence and a corresponding PBS as exemplified in Tables 20A-36B, wherein the 3′ PEgRNA comprises an editing template comprising a RTT sequence having a region of complementarity to the 5′ RTT. The 3′ RTT can comprise any of the RTT sequences provided in Table 38, wherein the 3′ RTT sequence has the same RTT Pairing number as that of the 5′ RTT. The editing template of the 5′ PEgRNA and the 3′ PEgRNA can be completely complementary to each other, wherein the 5′ editing template and the 3′ editing template have the same length and perfect complementarity to each other. Alternatively, the 5′ editing template can comprise a region of complementarity to the 3′ editing template, and further comprise a non-complementary region to the 3′ editing template. The 3′ editing template can comprise a region of complementarity to the 5′ editing template, and further comprise a non-complementary region to the 5′ editing template. The non-complementary region can be at the 5′ end or the 3′ end of the editing templates. Contacting the target DMPK gene with the prime editing system can result in deletion of the sequence between the 5′ PEgRNA nick and the 3′ PEgRNA nick (the IND) and insertion of the region of complementarity between the 5′ editing template and the 3′ editing template (the OD), or insertion of the region of complementarity between the 5′ editing template and the 3′ editing template as well as the non-complementarity regions (the RD), and hence replace the CTG expansion with the sequence of the OD or the RD. Each of the 5′ PEgRNA is assigned a RTT pairing number, and can be used in a dual prime editing composition with a 3′ PEgRNA comprising an RTT having the same RTT pairing number.

In some embodiments, a prime editing composition comprises a 5′ PEgRNA or component thereof exemplified in Tables 2A and 2B and a 3′ PEgRNA, wherein the editing template of the 5′ PEgRNA comprises a region of identity to the spacer of the 3′ PEgRNA. In some embodiments, the editing template of the 3′ PEgRNA comprises a region of identity to the spacer of the 5′ PEgRNA. In some embodiments, the 5′ editing template comprises a region of complementarity to a region on the second strand of the DMPK gene that is directly 3′ to nucleotide 3 of the search target sequence of the 3′ PEgRNA. In some embodiments, the 3′ editing template comprises a region of complementarity to a region on the first strand of the DMPK gene that is directly 3′ to nucleotide 3 of the search target sequence of the 5′ PEgRNA. For example, the extension arm of the 5′ PEgRNA can comprise an editing template comprising the sequence of any of the RTT sequences provided in Table 39. Each of the RTT sequences in Table 39 is assigned a pairing 3′ spacer number. The 5′ PEgRNA can be used in a dual prime editing system with a 3′ PEgRNA, wherein the 3′ PEgRNA comprises a spacer that has the Pairing 3′ spacer number assigned to the 5′ RTT in Table 39. Contacting the target DMPK gene with the prime editing system can result in deletion of the sequence between the 5′ PEgRNA nick and the 3′ PEgRNA nick (the IND) without insertion of any exogenous sequence (a “clean deletion”). Alternatively, the 5′ editing template and the 3′ editing template can further comprise a region of complementarity to each other, and the region of complementarity can replace the IND in the DMPK gene. For example, in some embodiments, the 5′ PEgRNA is part of a dual prime editing composition that further comprises a 3′ PEgRNA, wherein the 5′ PEgRNA comprises a RTT comprising a sequence selected from sequence numbers 3459-3461 and 3467-3471, and the 3′ PEgRNA comprises a spacer comprising nucleotides 5-20 of sequence number 1525. In some embodiments, the 5′ PEgRNA is part of a dual prime editing composition that further comprises a 3′ PEgRNA, wherein the 5′ PEgRNA comprises a RTT comprising a sequence selected from sequence numbers 3462-3466, and the 3′ PEgRNA comprises a spacer comprising nucleotides 5-20 of sequence number 2353. In some embodiments, the 5′ PEgRNA is part of a dual prime editing composition that further comprises a 3′ PEgRNA, wherein the 5′ PEgRNA comprises a RTT comprising a sequence selected from sequence numbers 3472-3476, and the 3′ PEgRNA comprises a spacer comprising nucleotides 5-20 of sequence number 1936. In some embodiments, the 5′ PEgRNA is part of a dual prime editing composition that further comprises a 3′ PEgRNA, wherein the 5′ PEgRNA comprises a RTT comprising nucleotides x-40 of sequence number 3647, 3648, 3649, 3650, 3651, 3652, 3653, 3654, 3655, 3656, 3657, 3658, 3659, 3660, 3661, 3662, or 3663, wherein the 3′ PEgRNA comprises a spacer comprising nucleotides 5-20 of sequence number 1900, 1918, 1403, 1936, 2263, 2353, 1503, 2281, 2673, 1525, 2299, 2317, 1864, 1359, 2335, 1882, or 1381 respectively in the same order, and wherein x is any integer from 1 to 31. Preferably, the 3′ PEgRNA comprises a “clean deletion” RTT, for example, an RTT selected from Table 40 that has a Pairing 5′ spacer number that is 2. Each 5′ PEgRNA is assigned a RTT pairing number, which corresponds to a pairing 3′ spacer number in Table 39. The 5′ PEgRNA can be used in a dual prime editing system or composition with a 3′ PEgRNA comprising a spacer of the same pairing 3′ spacer number.

### Tables 3A and 3B

Tables 3A and 3B provide exemplary 5′ PEgRNAs and components that can be used with any prime editor containing a Cas9 protein capable of recognizing a NGG PAM sequence, wherein N is A, G, C, or T. The PEgRNAs exemplified in Tables 3A and 3B comprise: (a) a spacer comprising at its 3′ end a sequence corresponding to nucleotides 5-20 of sequence number 39, (b) a gRNA core capable of binding to a Cas9 protein, and (c) an extension arm comprising (i) an editing template and (ii) a primer binding site (PBS) comprising at its 5′ end the sequence corresponding to sequence number 54.

The PEgRNA spacer can be, for example, 16-22 nucleotides in length. In some embodiments, the PEgNA spacer is 16-20 nucleotides in length. In some embodiments, the PEgRNA spacer comprises nucleotides 5-20, 4-20, 3-20, 2-20, or 1-20 of sequence number 39. In some embodiments, the PEgRNA spacer comprises the sequence of sequence number 39. In some embodiments, the PEgRNA spacer is 20 nucleotides in length. Any of these spacers exemplified can be referred to as 5′ spacer No. 3.

The PBS can be, for example, 3 to 19 nucleotides in length. In some embodiments, the PBS is 3 to 17 nucleotides in length and corresponds to a PBS sequence in Table 3A. In some cases, a PBS length of no more than 3 nucleotides less than the PEgRNA spacer length is chosen.

The 5′ PEgRNAs of Tables 3A and 3B can be used in a dual prime editing system or composition further comprising any suitable 3′ PEgRNA. The prime editing system can be used, e.g., for editing a DMPK gene harboring 50 or more CTG repeats in the DMPK gene.

In some embodiments, a prime editing composition comprises a 5′ PEgRNA or component thereof exemplified in Tables 3A and 3B and a 3′ PEgRNA, wherein the editing template of the 5′ PEgRNA and the 3′ PEgRNA each comprises a region of complementarity to each other. For example, the editing template of the 5′ PEgRNA can comprise any of the RTT sequences provided in Table 37. The terms editing template and RTT may be used interchangeably. Each of the RTT sequences in Table 37 is assigned a RTT pairing number that reflects the complementarity relationship between a 5′ RTT and a 3′ RTT. The 5′ PEgRNA can be used in a dual prime editing system that further comprises a 3′ PEgRNA, e.g., a 3′ PEgRNA having a spacer sequence and a corresponding PBS as exemplified in Tables 20A-36B, wherein the 3′ PEgRNA comprises an editing template comprising a RTT sequence having a region of complementarity to the 5′ RTT. The 3′ RTT can comprise any of the RTT sequences provided in Table 38, wherein the 3′ RTT sequence has the same RTT Pairing number as that of the 5′ RTT. The editing template of the 5′ PEgRNA and the 3′ PEgRNA can be completely complementary to each other, wherein the 5′ editing template and the 3′ editing template have the same length and perfect complementarity to each other. Alternatively, the 5′ editing template can comprise a region of complementarity to the 3′ editing template, and further comprise a non-complementary region to the 3′ editing template. The 3′ editing template can comprise a region of complementarity to the 5′ editing template, and further comprise a non-complementary region to the 5′ editing template. The non-complementary region can be at the PEgRNA nick (the IND) and insertion of the region of complementarity between the 5′ editing template and the 3′ editing template (the OD), or insertion of the region of complementarity between the 5′ editing template and the 3′ editing template as well as the non-complementarity regions (the RD), and hence replace the CTG expansion with the sequence of the OD or the RD. Each of the 5′ PEgRNA is assigned a RTT pairing number, and can be used in a dual prime editing composition with a 3′ PEgRNA comprising an RTT having the same RTT pairing number.

In some embodiments, a prime editing composition comprises a 5′ PEgRNA or component thereof exemplified in Tables 3A and 3B and a 3′ PEgRNA, wherein the editing template of the 5′ PEgRNA comprises a region of identity to the spacer of the 3′ PEgRNA. In some embodiments, the editing template of the 3′ PEgRNA comprises a region of identity to the spacer of the 5′ PEgRNA. In some embodiments, the 5′ editing template comprises a region of complementarity to a region on the second strand of the DMPK gene that is directly 3′ to nucleotide 3 of the search target sequence of the 3′ PEgRNA. In some embodiments, the 3′ editing template comprises a region of complementarity to a region on the first strand of the DMPK gene that is directly 3′ to nucleotide 3 of the search target sequence of the 5′ PEgRNA. For example, the extension arm of the 5′ PEgRNA can comprise an editing template comprising the sequence of any of the RTT sequences provided in Table 39. Each of the RTT sequences in Table 39 is assigned a pairing 3′ spacer number. The 5′ PEgRNA can be used in a dual prime editing system with a 3′ PEgRNA, wherein the 3′ PEgRNA comprises a spacer that has the Pairing 3′ spacer number assigned to the 5′ RTT in Table 39. Contacting the target DMPK gene with the prime editing system can result in deletion of the sequence between the 5′ PEgRNA nick and the 3′ PEgRNA nick (the IND) without insertion of any exogenous sequence (a “clean deletion”). Alternatively, the 5′ editing template and the 3′ editing template can further comprise a region of complementarity to each other, and the region of complementarity can replace the IND in the DMPK gene. For example, in some embodiments, the 5′ PEgRNA is part of a dual prime editing composition that further comprises a 3′ PEgRNA, wherein the 5′ PEgRNA comprises a RTT comprising a sequence selected from sequence numbers 3459-3461 and 3467-3471, and the 3′ PEgRNA comprises a spacer comprising nucleotides 5-20 of sequence number 1525. In some embodiments, the 5′ PEgRNA is part of a dual prime editing composition that further comprises a 3′ PEgRNA, wherein the 5′ PEgRNA comprises a RTT comprising a sequence selected from sequence numbers 3462-3466, and the 3′ PEgRNA comprises a spacer comprising nucleotides 5-20 of sequence number 2353. In some embodiments, the 5′ PEgRNA is part of a dual prime editing composition that further comprises a 3′ PEgRNA, wherein the 5′ PEgRNA comprises a RTT comprising a sequence selected from sequence numbers 3472-3476, and the 3′ PEgRNA comprises a spacer comprising nucleotides 5-20 of sequence number 1936. In some embodiments, the 5′ PEgRNA is part of a dual prime editing composition that further comprises a 3′ PEgRNA, wherein the 5′ PEgRNA comprises a RTT comprising nucleotides x-40 of sequence number 3647, 3648, 3649, 3650, 3651, 3652, 3653, 3654, 3655, 3656, 3657, 3658, 3659, 3660, 3661, 3662, or 3663, wherein the 3′ PEgRNA comprises a spacer comprising nucleotides 5-20 of sequence number 1900, 1918, 1403, 1936, 2263, 2353, 1503, 2281, 2673, 1525, 2299, 2317, 1864, 1359, 2335, 1882, or 1381 respectively in the same order, and wherein x is any integer from 1 to 31. Preferably, the 3′ PEgRNA comprises a “clean deletion” RTT, for example, an RTT selected from Table 40 that has a Pairing 5′ spacer number that is 3. Each 5′ PEgRNA is assigned a RTT pairing number, which corresponds to a pairing 3′ spacer number in Table 39. The 5′ PEgRNA can be used in a dual prime editing system or composition with a 3′ PEgRNA comprising a spacer of the same pairing 3′ spacer number.

### Tables 4A and 4B

Tables 4A and 4B provide exemplary 5′ PEgRNAs and components that can be used with any prime editor containing a Cas9 protein capable of recognizing a NGG PAM sequence, wherein N is A, G, C, or T. The PEgRNAs exemplified in Tables 4A and 4B comprise: (a) a spacer comprising at its 3′ end a sequence corresponding to nucleotides 5-20 of sequence number 58, (b) a gRNA core capable of binding to a Cas9 protein, and (c) an extension arm comprising (i) an editing template and (ii) a primer binding site (PBS) comprising at its 5′ end the sequence corresponding to sequence number 73.

The PEgRNA spacer can be, for example, 16-22 nucleotides in length. In some embodiments, the PEgNA spacer is 16-20 nucleotides in length. In some embodiments, the PEgRNA spacer comprises nucleotides 5-20, 4-20, 3-20, 2-20, or 1-20 of sequence number 58. In some embodiments, the PEgRNA spacer comprises the sequence of sequence number 58. In some embodiments, the PEgRNA spacer is 20 nucleotides in length. Any of these spacers exemplified can be referred to as 5′ spacer No. 4.

The PBS can be, for example, 3 to 19 nucleotides in length. In some embodiments, the PBS is 3 to 17 nucleotides in length and corresponds to a PBS sequence in Table 4A. In some cases, a PBS length of no more than 3 nucleotides less than the PEgRNA spacer length is chosen.

The 5′ PEgRNAs of Tables 4A and 4B can be used in a dual prime editing system or composition further comprising any suitable 3′ PEgRNA. The prime editing system can be used, e.g., for editing a DMPK gene harboring 50 or more CTG repeats in the DMPK gene.

In some embodiments, a prime editing composition comprises a 5′ PEgRNA or component thereof exemplified in Tables 4A and 4B and a 3′ PEgRNA, wherein the editing template of the 5′ PEgRNA and the 3′ PEgRNA each comprises a region of complementarity to each other. For example, the editing template of the 5′ PEgRNA can comprise any of the RTT sequences provided in Table 37. The terms editing template and RTT may be used interchangeably. Each of the RTT sequences in Table 37 is assigned a RTT pairing number that reflects the complementarity relationship between a 5′ RTT and a 3′ RTT. The 5′ PEgRNA can be used in a dual prime editing system that further comprises a 3′ PEgRNA, e.g., a 3′ PEgRNA having a spacer sequence and a corresponding PBS as exemplified in Tables 20A-36B, wherein the 3′ PEgRNA comprises an editing template comprising a RTT sequence having a region of complementarity to the 5′ RTT. The 3′ RTT can comprise any of the RTT sequences provided in Table 38, wherein the 3′ RTT sequence has the same RTT Pairing number as that of the 5′ RTT. The editing template of the 5′ PEgRNA and the 3′ PEgRNA can be completely complementary to each other, wherein the 5′ editing template and the 3′ editing template have the same length and perfect complementarity to each other. Alternatively, the 5′ editing template can comprise a region of complementarity to the 3′ editing template, and further comprise a non-complementary region to the 3′ editing template. The 3′ editing template can comprise a region of complementarity to the 5′ editing template, and further comprise a non-complementary region to the 5′ editing template. The non-complementary region can be at the regions (the RD), and hence replace the CTG expansion with the sequence of the OD or the RD. Each of the 5′ PEgRNA is assigned a RTT pairing number, and can be used in a dual prime editing composition with a 3′ PEgRNA comprising an RTT having the same RTT pairing number.

In some embodiments, a prime editing composition comprises a 5′ PEgRNA or component thereof exemplified in Tables 4A and 4B and a 3′ PEgRNA, wherein the editing template of the 5′ PEgRNA comprises a region of identity to the spacer of the 3′ PEgRNA. In some embodiments, the editing template of the 3′ PEgRNA comprises a region of identity to the spacer of the 5′ PEgRNA. In some embodiments, the 5′ editing template comprises a region of complementarity to a region on the second strand of the DMPK gene that is directly 3′ to nucleotide 3 of the search target sequence of the 3′ PEgRNA. In some embodiments, the 3′ editing template comprises a region of complementarity to a region on the first strand of the DMPK gene that is directly 3′ to nucleotide 3 of the search target sequence of the 5′ PEgRNA. For example, the extension arm of the 5′ PEgRNA can comprise an editing template comprising the sequence of any of the RTT sequences provided in Table 39. Each of the RTT sequences in Table 39 is assigned a pairing 3′ spacer number. The 5′ PEgRNA can be used in a dual prime editing system with a 3′ PEgRNA, wherein the 3′ PEgRNA comprises a spacer that has the Pairing 3′ spacer number assigned to the 5′ RTT in Table 39. Contacting the target DMPK gene with the prime editing system can result in deletion of the sequence between the 5′ PEgRNA nick and the 3′ PEgRNA nick (the IND) without insertion of any exogenous sequence (a “clean deletion”). Alternatively, the 5′ editing template and the 3′ editing template can further comprise a region of complementarity to each other, and the region of complementarity can replace the IND in the DMPK gene. For example, in some embodiments, the 5′ PEgRNA is part of a dual prime editing composition that further comprises a 3′ PEgRNA, wherein the 5′ PEgRNA comprises a RTT comprising a sequence selected from sequence numbers 3459-3461 and 3467-3471, and the 3′ PEgRNA comprises a spacer comprising nucleotides 5-20 of sequence number 1525. In some embodiments, the 5′ PEgRNA is part of a dual prime editing composition that further comprises a 3′ PEgRNA, wherein the 5′ PEgRNA comprises a RTT comprising a sequence selected from sequence numbers 3462-3466, and the 3′ PEgRNA comprises a spacer comprising nucleotides 5-20 of sequence number 2353. In some embodiments, the 5′ PEgRNA is part of a dual prime editing composition that further comprises a 3′ PEgRNA, wherein the 5′ PEgRNA comprises a RTT comprising a sequence selected from sequence numbers 3472-3476, and the 3′ PEgRNA comprises a spacer comprising nucleotides 5-20 of sequence number 1936. In some embodiments, the 5′ PEgRNA is part of a dual prime editing composition that further comprises a 3′ PEgRNA, wherein the 5′ PEgRNA comprises a RTT comprising nucleotides x-40 of sequence number 3647, 3648, 3649, 3650, 3651, 3652, 3653, 3654, 3655, 3656, 3657, 3658, 3659, 3660, 3661, 3662, or 3663, wherein the 3′ PEgRNA comprises a spacer comprising nucleotides 5-20 of sequence number 1900, 1918, 1403, 1936, 2263, 2353, 1503, 2281, 2673, 1525, 2299, 2317, 1864, 1359, 2335, 1882, or 1381 respectively in the same order, and wherein x is any integer from 1 to 31. Preferably, the 3′ PEgRNA comprises a “clean deletion” RTT, for example, an RTT selected from Table 40 that has a Pairing 5′ spacer number that is 4. Each 5′ PEgRNA is assigned a RTT pairing number, which corresponds to a pairing 3′ spacer number in Table 39. The 5′ PEgRNA can be used in a dual prime editing system or composition with a 3′ PEgRNA comprising a spacer of the same pairing 3′ spacer number.

### Tables 6A and 6B

Tables 6A and 6B provide exemplary 5′ PEgRNAs and components that can be used with any prime editor containing a Cas9 protein capable of recognizing a NGG PAM sequence, wherein N is A, G, C, or T. The PEgRNAs exemplified in Tables 6A and 6B comprise: (a) a spacer comprising at its 3′ end a sequence corresponding to nucleotides 5-20 of sequence number 484, (b) a gRNA core capable of binding to a Cas9 protein, and (c) an extension arm comprising (i) an editing template and (ii) a primer binding site (PBS) comprising at its 5′ end the sequence corresponding to sequence number 499.

The PEgRNA spacer can be, for example, 16-22 nucleotides in length. In some embodiments, the PEgNA spacer is 16-20 nucleotides in length. In some embodiments, the PEgRNA spacer comprises nucleotides 5-20, 4-20, 3-20, 2-20, or 1-20 of sequence number 484. In some embodiments, the PEgRNA spacer comprises the sequence of sequence number 484. In some embodiments, the PEgRNA spacer is 20 nucleotides in length. Any of these spacers exemplified can be referred to as 5′ spacer No. 6.

The PBS can be, for example, 3 to 19 nucleotides in length. In some embodiments, the PBS is 3 to 17 nucleotides in length and corresponds to a PBS sequence in Table 6A. In some cases, a PBS length of no more than 3 nucleotides less than the PEgRNA spacer length is chosen.

The 5′ PEgRNAs of Tables 6A and 6B can be used in a dual prime editing system or composition further comprising any suitable 3′ PEgRNA. The prime editing system can be used, e.g., for editing a DMPK gene harboring 50 or more CTG repeats in the DMPK gene.

In some embodiments, a prime editing composition comprises a 5′ PEgRNA or component thereof exemplified in Tables 6A and 6B and a 3′ PEgRNA, wherein the editing template of the 5′ PEgRNA and the 3′ PEgRNA each comprises a region of complementarity to each other. For example, the editing template of the 5′ PEgRNA can comprise any of the RTT sequences provided in Table 37. The terms editing template and RTT may be used interchangeably. Each of the RTT sequences in Table 37 is assigned a RTT pairing number that reflects the complementarity relationship between a 5′ RTT and a 3′ RTT. The 5′ PEgRNA can be used in a dual prime editing system that further comprises a 3′ PEgRNA, e.g., a 3′ PEgRNA having a spacer sequence and a corresponding PBS as exemplified in Tables 20A-36B, wherein the 3′ PEgRNA comprises an editing template comprising a RTT sequence having a region of complementarity to the 5′ RTT. The 3′ RTT can comprise any of the RTT sequences provided in Table 38, wherein the 3′ RTT sequence has the same RTT Pairing number as that of the 5′ RTT. The editing template of the 5′ PEgRNA and the 3′ PEgRNA can be completely complementary to each other, wherein the 5′ editing template and the 3′ editing template have the same length and perfect complementarity to each other. Alternatively, the 5′ editing template can comprise a region of complementarity to the 3′ editing template, and further comprise a non-complementary region to the 3′ editing template. The 3′ editing template can comprise a region of complementarity to the 5′ editing template, and further comprise a non-complementary region to the 5′ editing template. The non-complementary region can be at the 5′ end or the 3′ end of the editing templates. Contacting the target DMPK gene with the prime editing system can result in deletion of the sequence between the 5′ PEgRNA nick and the 3′ PEgRNA nick (the IND) and insertion of the region of complementarity between the 5′ editing template and the 3′ editing template (the OD), or insertion of the region of complementarity between the 5′ editing template and the 3′ editing template as well as the non-complementarity regions (the RD), and hence replace the CTG expansion with the sequence of the OD or the RD. Each of the 5′ PEgRNA is assigned a RTT pairing number, and can be used in a dual prime editing composition with a 3′ PEgRNA comprising an RTT having the same RTT pairing number.

In some embodiments, a prime editing composition comprises a 5′ PEgRNA or component thereof exemplified in Tables 6A and 6B and a 3′ PEgRNA, wherein the editing template of the 5′ PEgRNA comprises a region of identity to the spacer of the 3′ PEgRNA. In some embodiments, the editing template of the 3′ PEgRNA comprises a region of identity to the spacer of the 5′ PEgRNA. In some embodiments, the 5′ editing template comprises a region of complementarity to a region on the second strand of the DMPK gene that is directly 3′ to nucleotide 3 of the search target sequence of the 3′ PEgRNA. In some embodiments, the 3′ editing template comprises a region of complementarity to a region on the first strand of the DMPK gene that is directly 3′ to nucleotide 3 of the search target sequence of the 5′ PEgRNA. For example, the extension arm of the 5′ PEgRNA can comprise an editing template comprising the sequence of any of the RTT sequences provided in Table 39. Each of the RTT sequences in Table 39 is assigned a pairing 3′ spacer number. The 5′ PEgRNA can be used in a dual prime editing system with a 3′ PEgRNA, wherein the 3′ PEgRNA comprises a spacer that has the Pairing 3′ spacer number assigned to the 5′ RTT in Table 39. Contacting the target DMPK gene with the prime editing system can result in deletion of the sequence between the 5′ PEgRNA nick and the 3′ PEgRNA nick (the IND) without insertion of any exogenous sequence (a “clean deletion”). Alternatively, the 5′ editing template and the 3′ editing template can further comprise a region of complementarity to each other, and the region of complementarity can replace the IND in the DMPK gene. For example, in some embodiments, the 5′ PEgRNA is part of a dual prime editing composition that further comprises a 3′ PEgRNA, wherein the 5′ PEgRNA comprises a RTT comprising a sequence selected from sequence numbers 3459-3461 and 3467-3471, and the 3′ PEgRNA comprises a spacer comprising nucleotides 5-20 of sequence number 1525. In some embodiments, the 5′ PEgRNA is part of a dual prime editing composition that further comprises a 3′ PEgRNA, wherein the 5′ PEgRNA comprises a RTT comprising a sequence selected from sequence numbers 3462-3466, and the 3′ PEgRNA comprises a spacer comprising nucleotides 5-20 of sequence number 2353. In some embodiments, the 5′ PEgRNA is part of a dual prime editing composition that further comprises a 3′ PEgRNA, wherein the 5′ PEgRNA comprises a RTT comprising a sequence selected from sequence numbers 3472-3476, and the 3′ PEgRNA comprises a spacer comprising nucleotides 5-20 of sequence number 1936. In some embodiments, the 5′ PEgRNA is part of a dual prime editing composition that further comprises a 3′ PEgRNA, wherein the 5′ PEgRNA comprises a RTT comprising nucleotides x-40 of sequence number 3647, 3648, 3649, 3650, 3651, 3652, 3653, 3654, 3655, 3656, 3657, 3658, 3659, 3660, 3661, 3662, or 3663, wherein the 3′ PEgRNA comprises a spacer comprising nucleotides 5-20 of sequence number 1900, 1918, 1403, 1936, 2263, 2353, 1503, 2281, 2673, 1525, 2299, 2317, 1864, 1359, 2335, 1882, or 1381 respectively in the same order, and wherein x is any integer from 1 to 31. Preferably, the 3′ PEgRNA comprises a “clean deletion” RTT, for example, an RTT selected from Table 40 that has a Pairing 5′ spacer number that is 6. Each 5′ PEgRNA is assigned a RTT pairing number, which corresponds to a pairing 3′ spacer number in Table 39. The 5′ PEgRNA can be used in a dual prime editing system or composition with a 3′ PEgRNA comprising a spacer of the same pairing 3′ spacer number.

### Tables 7A and 7B

Tables 7A and 7B provide exemplary 5′ PEgRNAs and components that can be used with any prime editor containing a Cas9 protein capable of recognizing a NGG PAM sequence, wherein N is A, G, C, or T. The PEgRNAs exemplified in Tables 7A and 7B comprise: (a) a spacer comprising at its 3′ end a sequence corresponding to nucleotides 5-20 of sequence number 518, (b) a gRNA core capable of binding to a Cas9 protein, and (c) an extension arm comprising (i) an editing template and (ii) a primer binding site (PBS) comprising at its 5′ end the sequence corresponding to sequence number 533.

The PEgRNA spacer can be, for example, 16-22 nucleotides in length. In some embodiments, the PEgNA spacer is 16-20 nucleotides in length. In some embodiments, the PEgRNA spacer comprises nucleotides 5-20, 4-20, 3-20, 2-20, or 1-20 of sequence number 518. In some embodiments, the PEgRNA spacer comprises the sequence of sequence number 518. In some embodiments, the PEgRNA spacer is 20 nucleotides in length. Any of these spacers exemplified can be referred to as 5′ spacer No. 7.

The PBS can be, for example, 3 to 19 nucleotides in length. In some embodiments, the PBS is 3 to 17 nucleotides in length and corresponds to a PBS sequence in Table 7A. In some cases, a PBS length of no more than 3 nucleotides less than the PEgRNA spacer length is chosen.

The 5′ PEgRNAs of Tables 7A and 7B can be used in a dual prime editing system or composition further comprising any suitable 3′ PEgRNA. The prime editing system can be used, e.g., for editing a DMPK gene harboring 50 or more CTG repeats in the DMPK gene.

In some embodiments, a prime editing composition comprises a 5′ PEgRNA or component thereof exemplified in Tables 7A and 7B and a 3′ PEgRNA, wherein the editing template of the 5′ PEgRNA and the 3′ PEgRNA each comprises a region of complementarity to each other. For example, the editing template of the 5′ PEgRNA can comprise any of the RTT sequences provided in Table 37. The terms editing template and RTT may be used interchangeably. Each of the RTT sequences in Table 37 is assigned a RTT pairing number that reflects the complementarity relationship between a 5′ RTT and a 3′ RTT. The 5′ PEgRNA can be used in a dual prime editing system that further comprises a 3′ PEgRNA, e.g., a 3′ PEgRNA having a spacer sequence and a corresponding PBS as exemplified in Tables 20A-36B, wherein the 3′ PEgRNA comprises an editing template comprising a RTT sequence having a region of complementarity to the 5′ RTT. The 3′ RTT can comprise any of the RTT sequences provided in Table 38, wherein the 3′ RTT sequence has the same RTT Pairing number as that of the 5′ RTT. The editing template of the 5′ PEgRNA and the 3′ PEgRNA can be completely complementary to each other, wherein the 5′ editing template and the 3′ editing template have the same length and perfect complementarity to each other. Alternatively, the 5′ editing template can comprise a region of complementarity to the 3′ editing template, and further comprise a non-complementary region to the 3′ editing template. The 3′ editing template can comprise a region of complementarity to the 5′ editing template, and further comprise a non-complementary region to the 5′ editing template. The non-complementary region can be at the 5′ end or the 3′ end of the editing templates. Contacting the target DMPK gene with the prime editing system can result in deletion of the sequence between the 5′ PEgRNA nick and the 3′ PEgRNA nick (the IND) and insertion of the region of complementarity between the 5′ editing template and the 3′ editing template (the OD), or insertion of the region of complementarity between the 5′ editing template and the 3′ editing template as well as the non-complementarity regions (the RD), and hence replace the CTG expansion with the sequence of the OD or the RD. Each of the 5′ PEgRNA is assigned a RTT pairing number, and can be used in a dual prime editing composition with a 3′ PEgRNA comprising an RTT having the same RTT pairing number.

In some embodiments, a prime editing composition comprises a 5′ PEgRNA or component thereof exemplified in Tables 7A and 7B and a 3′ PEgRNA, wherein the editing template of the 5′ PEgRNA comprises a region of identity to the spacer of the 3′ PEgRNA. In some embodiments, the editing template of the 3′ PEgRNA comprises a region of identity to the spacer of the 5′ PEgRNA. In some embodiments, the 5′ editing template comprises a region of complementarity to a region on the second strand of the DMPK gene that is directly 3′ to nucleotide 3 of the search target sequence of the 3′ PEgRNA. In some embodiments, the 3′ editing template comprises a region of complementarity to a region on the first strand of the DMPK gene that is directly 3′ to nucleotide 3 of the search target sequence of the 5′ PEgRNA. For example, the extension arm of the 5′ PEgRNA can comprise an editing template comprising the sequence of any of the RTT sequences provided in Table 39. Each of the RTT sequences in Table 39 is assigned a pairing 3′ spacer number. The 5′ PEgRNA can be used in a dual prime editing system with a 3′ PEgRNA, wherein the 3′ PEgRNA comprises a spacer that has the Pairing 3′ spacer number assigned to the 5′ RTT in Table 39. Contacting the target DMPK gene with the prime editing system can result in deletion of the sequence between the 5′ PEgRNA nick and the 3′ PEgRNA nick (the IND) without insertion of any exogenous sequence (a “clean deletion”). Alternatively, the 5′ editing template and the 3′ editing template can further comprise a region of complementarity to each other, and the region of complementarity can replace the IND in the DMPK gene. For example, in some embodiments, the 5′ PEgRNA is part of a dual prime editing composition that further comprises a 3′ PEgRNA, wherein the 5′ PEgRNA comprises a RTT comprising a sequence selected from sequence numbers 3459-3461 and 3467-3471, and the 3′ PEgRNA comprises a spacer comprising nucleotides 5-20 of sequence number 1525. In some embodiments, the 5′ PEgRNA is part of a dual prime editing composition that further comprises a 3′ PEgRNA, wherein the 5′ PEgRNA comprises a RTT comprising a sequence selected from sequence numbers 3462-3466, and the 3′ PEgRNA comprises a spacer comprising nucleotides 5-20 of sequence number 2353. In some embodiments, the 5′ PEgRNA is part of a dual prime editing composition that further comprises a 3′ PEgRNA, wherein the 5′ PEgRNA comprises a RTT comprising a sequence selected from sequence numbers 3472-3476, and the 3′ PEgRNA comprises a spacer comprising nucleotides 5-20 of sequence number 1936. In some embodiments, the 5′ PEgRNA is part of a dual prime editing composition that further comprises a 3′ PEgRNA, wherein the 5′ PEgRNA comprises a RTT comprising nucleotides x-40 of sequence number 3647, 3648, 3649, 3650, 3651, 3652, 3653, 3654, 3655, 3656, 3657, 3658, 3659, 3660, 3661, 3662, or 3663, wherein the 3′ PEgRNA comprises a spacer comprising nucleotides 5-20 of sequence number 1900, 1918, 1403, 1936, 2263, 2353, 1503, 2281, 2673, 1525, 2299, 2317, 1864, 1359, 2335, 1882, or 1381 respectively in the same order, and wherein x is any integer from 1 to 31. Preferably, the 3′ PEgRNA comprises a “clean deletion” RTT, for example, an RTT selected from Table 40 that has a Pairing 5′ spacer number that is 7. Each 5′ PEgRNA is assigned a RTT pairing number, which corresponds to a pairing 3′ spacer number in Table 39. The 5′ PEgRNA can be used in a dual prime editing system or composition with a 3′ PEgRNA comprising a spacer of the same pairing 3′ spacer number.

### Tables 9A and 9B

Tables 9A and 9B provide exemplary 5′ PEgRNAs and components that can be used with any prime editor containing a Cas9 protein capable of recognizing a NGG PAM sequence, wherein N is A, G, C, or T. The PEgRNAs exemplified in Tables 9A and 9B comprise: (a) a spacer comprising at its 3′ end a sequence corresponding to nucleotides 5-20 of sequence number 554, (b) a gRNA core capable of binding to a Cas9 protein, and (c) an extension arm comprising (i) an editing template and (ii) a primer binding site (PBS) comprising at its 5′ end the sequence corresponding to sequence number 569.

The PEgRNA spacer can be, for example, 16-22 nucleotides in length. In some embodiments, the PEgNA spacer is 16-20 nucleotides in length. In some embodiments, the PEgRNA spacer comprises nucleotides 5-20, 4-20, 3-20, 2-20, or 1-20 of sequence number 554. In some embodiments, the PEgRNA spacer comprises the sequence of sequence number 554. In some embodiments, the PEgRNA spacer is 20 nucleotides in length. Any of these spacers exemplified can be referred to as 5′ spacer No. 9.

The PBS can be, for example, 3 to 19 nucleotides in length. In some embodiments, the PBS is 3 to 17 nucleotides in length and corresponds to a PBS sequence in Table 9A. In some cases, a PBS length of no more than 3 nucleotides less than the PEgRNA spacer length is chosen.

The 5′ PEgRNAs of Tables 9A and 9B can be used in a dual prime editing system or composition further comprising any suitable 3′ PEgRNA. The prime editing system can be used, e.g., for editing a DMPK gene harboring 50 or more CTG repeats in the DMPK gene.

In some embodiments, a prime editing composition comprises a 5′ PEgRNA or component thereof exemplified in Tables 9A and 9B and a 3′ PEgRNA, wherein the editing template of the 5′ PEgRNA and the 3′ PEgRNA each comprises a region of complementarity to each other. For example, the editing template of the 5′ PEgRNA can comprise any of the RTT sequences provided in Table 37. The terms editing template and RTT may be used interchangeably. Each of the RTT sequences in Table 37 is assigned a RTT pairing number that reflects the complementarity relationship between a 5′ RTT and a 3′ RTT. The 5′ PEgRNA can be used in a dual prime editing system that further comprises a 3′ PEgRNA, e.g., a 3′ PEgRNA having a spacer sequence and a corresponding PBS as exemplified in Tables 20A-36B, wherein the 3′ PEgRNA comprises an editing template comprising a RTT sequence having a region of complementarity to the 5′ RTT. The 3′ RTT can comprise any of the RTT sequences provided in Table 38, wherein the 3′ RTT sequence has the same RTT Pairing number as that of the 5′ RTT. The editing template of the 5′ PEgRNA and the 3′ PEgRNA can be completely complementary to each other, wherein the 5′ editing template and the 3′ editing template have the same length and perfect complementarity to each other. Alternatively, the 5′ editing template can comprise a region of complementarity to the 3′ editing template, and further comprise a non-complementary region to the 3′ editing template. The 3′ editing template can comprise a region of complementarity to the 5′ editing template, and further comprise a non-complementary region to the 5′ editing template. The non-complementary region can be at the 5′ end or the 3′ end of the editing templates. Contacting the target DMPK gene with the prime editing system can result in deletion of the sequence between the 5′ PEgRNA nick and the 3′ PEgRNA nick (the IND) and insertion of the region of complementarity between the 5′ editing template and the 3′ editing template (the OD), or insertion of the region of complementarity between the 5′ editing template and the 3′ editing template as well as the non-complementarity regions (the RD), and hence replace the CTG expansion with the sequence of the OD or the RD. Each of the 5′ PEgRNA is assigned a RTT pairing number, and can be used in a dual prime editing composition with a 3′ PEgRNA comprising an RTT having the same RTT pairing number.

In some embodiments, a prime editing composition comprises a 5′ PEgRNA or component thereof exemplified in Tables 9A and 9B and a 3′ PEgRNA, wherein the editing template of the 5′ PEgRNA comprises a region of identity to the spacer of the 3′ PEgRNA. In some embodiments, the editing template of the 3′ PEgRNA comprises a region of identity to the spacer of the 5′ PEgRNA. In some embodiments, the 5′ editing template comprises a region of complementarity to a region on the second strand of the DMPK gene that is directly 3′ to nucleotide 3 of the search target sequence of the 3′ PEgRNA. In some embodiments, the 3′ editing template comprises a region of complementarity to a region on the first strand of the DMPK gene that is directly 3′ to nucleotide 3 of the search target sequence of the 5′ PEgRNA. For example, the extension arm of the 5′ PEgRNA can comprise an editing template comprising the sequence of any of the RTT sequences provided in Table 39. Each of the RTT sequences in Table 39 is assigned a pairing 3′ spacer number. The 5′ PEgRNA can be used in a dual prime editing system with a 3′ PEgRNA, wherein the 3′ PEgRNA comprises a spacer that has the Pairing 3′ spacer number assigned to the 5′ RTT in Table 39. Contacting the target DMPK gene with the prime editing system can result in deletion of the sequence between the 5′ PEgRNA nick and the 3′ PEgRNA nick (the IND) without insertion of any exogenous sequence (a “clean deletion”). Alternatively, the 5′ editing template and the 3′ editing template can further comprise a region of complementarity to each other, and the region of complementarity can replace the IND in the DMPK gene. For example, in some embodiments, the 5′ PEgRNA is part of a dual prime editing composition that further comprises a 3′ PEgRNA, wherein the 5′ PEgRNA comprises a RTT comprising a sequence selected from sequence numbers 3459-3461 and 3467-3471, and the 3′ PEgRNA comprises a spacer comprising nucleotides 5-20 of sequence number 1525. In some embodiments, the 5′ PEgRNA is part of a dual prime editing composition that further comprises a 3′ PEgRNA, wherein the 5′ PEgRNA comprises a RTT comprising a sequence selected from sequence numbers 3462-3466, and the 3′ PEgRNA comprises a spacer comprising nucleotides 5-20 of sequence number 2353. In some embodiments, the 5′ PEgRNA is part of a dual prime editing composition that further comprises a 3′ PEgRNA, wherein the 5′ PEgRNA comprises a RTT comprising a sequence selected from sequence numbers 3472-3476, and the 3′ PEgRNA comprises a spacer comprising nucleotides 5-20 of sequence number 1936. In some embodiments, the 5′ PEgRNA is part of a dual prime editing composition that further comprises a 3′ PEgRNA, wherein the 5′ PEgRNA comprises a RTT comprising nucleotides x-40 of sequence number 3647, 3648, 3649, 3650, 3651, 3652, 3653, 3654, 3655, 3656, 3657, 3658, 3659, 3660, 3661, 3662, or 3663, wherein the 3′ PEgRNA comprises a spacer comprising nucleotides 5-20 of sequence number 1900, 1918, 1403, 1936, 2263, 2353, 1503, 2281, 2673, 1525, 2299, 2317, 1864, 1359, 2335, 1882, or 1381 respectively in the same order, and wherein x is any integer from 1 to 31. Preferably, the 3′ PEgRNA comprises a “clean deletion” RTT, for example, an RTT selected from Table 40 that has a Pairing 5′ spacer number that is 9. Each 5′ PEgRNA is assigned a RTT pairing number, which corresponds to a pairing 3′ spacer number in Table 39. The 5′ PEgRNA can be used in a dual prime editing system or composition with a 3′ PEgRNA comprising a spacer of the same pairing 3′ spacer number.

### Tables 10A and 10B

Tables 10A and 10B provide exemplary 5′ PEgRNAs and components that can be used with any prime editor containing a Cas9 protein capable of recognizing a NGG PAM sequence, wherein N is A, G, C, or T. The PEgRNAs exemplified in Tables 10A and 10B comprise: (a) a spacer comprising at its 3′ end a sequence corresponding to nucleotides 5-20 of sequence number 572, (b) a gRNA core capable of binding to a Cas9 protein, and (c) an extension arm comprising (i) an editing template and (ii) a primer binding site (PBS) comprising at its 5′ end the sequence corresponding to sequence number 587.

The PEgRNA spacer can be, for example, 16-22 nucleotides in length. In some embodiments, the PEgNA spacer is 16-20 nucleotides in length. In some embodiments, the PEgRNA spacer comprises nucleotides 5-20, 4-20, 3-20, 2-20, or 1-20 of sequence number 572. In some embodiments, the PEgRNA spacer comprises the sequence of sequence number 572. In some embodiments, the PEgRNA spacer is 20 nucleotides in length. Any of these spacers exemplified can be referred to as 5′ spacer No. 10.

The PBS can be, for example, 3 to 19 nucleotides in length. In some embodiments, the PBS is 3 to 17 nucleotides in length and corresponds to a PBS sequence in Table 10A. In some cases, a PBS length of no more than 3 nucleotides less than the PEgRNA spacer length is chosen.

The 5′ PEgRNAs of Tables 10A and 10B can be used in a dual prime editing system or composition further comprising any suitable 3′ PEgRNA. The prime editing system can be used, e.g., for editing a DMPK gene harboring 50 or more CTG repeats in the DMPK gene.

In some embodiments, a prime editing composition comprises a 5′ PEgRNA or component thereof exemplified in Tables 10A and 10B and a 3′ PEgRNA, wherein the editing template of the 5′ PEgRNA and the 3′ PEgRNA each comprises a region of complementarity to each other. For example, the editing template of the 5′ PEgRNA can comprise any of the RTT sequences provided in Table 37. The terms editing template and RTT may be used interchangeably. Each of the RTT sequences in Table 37 is assigned a RTT pairing number that reflects the complementarity relationship between a 5′ RTT and a 3′ RTT. The 5′ PEgRNA can be used in a dual prime editing system that further comprises a 3′ PEgRNA, e.g., a 3′ PEgRNA having a spacer sequence and a corresponding PBS as exemplified in Tables 20A-36B, wherein the 3′ PEgRNA comprises an editing template comprising a RTT sequence having a region of complementarity to the 5′ RTT. The 3′ RTT can comprise any of the RTT sequences provided in Table 38, wherein the 3′ RTT sequence has the same RTT Pairing number as that of the 5′ RTT. The editing template of the 5′ PEgRNA and the 3′ PEgRNA can be completely complementary to each other, wherein the 5′ editing template and the 3′ editing template have the same length and perfect complementarity to each other. Alternatively, the 5′ editing template can comprise a region of complementarity to the 3′ editing template, and further comprise a non-complementary region to the 3′ editing template. The 3′ editing template can comprise a region of complementarity to the 5′ editing template, and further comprise a non-complementary region to the 5′ editing template. The non-complementary region can be at the 5′ end or the 3′ end of the editing templates. Contacting the target DMPK gene with the prime editing system can result in deletion of the sequence between the 5′ PEgRNA nick and the 3′ PEgRNA nick (the IND) and insertion of the region of complementarity between the 5′ editing template and the 3′ editing template (the OD), or insertion of the region of complementarity between the 5′ editing template and the 3′ editing template as well as the non-complementarity regions (the RD), and hence replace the CTG expansion with the sequence of the OD or the RD. Each of the 5′ PEgRNA is assigned a RTT pairing number, and can be used in a dual prime editing composition with a 3′ PEgRNA comprising an RTT having the same RTT pairing number.

In some embodiments, a prime editing composition comprises a 5′ PEgRNA or component thereof exemplified in Tables 10A and 10B and a 3′ PEgRNA, wherein the editing template of the 5′ PEgRNA comprises a region of identity to the spacer of the 3′ PEgRNA. In some embodiments, the editing template of the 3′ PEgRNA comprises a region of identity to the spacer of the 5′ PEgRNA. In some embodiments, the 5′ editing template comprises a region of complementarity to a region on the second strand of the DMPK gene that is directly 3′ to nucleotide 3 of the search target sequence of the 3′ PEgRNA. In some embodiments, the 3′ editing template comprises a region of complementarity to a region on the first strand of the DMPK gene that is directly 3′ to nucleotide 3 of the search target sequence of the 5′ PEgRNA. For example, the extension arm of the 5′ PEgRNA can comprise an editing template comprising the sequence of any of the RTT sequences provided in Table 39. Each of the RTT sequences in Table 39 is assigned a pairing 3′ spacer number. The 5′ PEgRNA can be used in a dual prime editing system with a 3′ PEgRNA, wherein the 3′ PEgRNA comprises a spacer that has the Pairing 3′ spacer number assigned to the 5′ RTT in Table 39. Contacting the target DMPK gene with the prime editing system can result in deletion of the sequence between the 5′ PEgRNA nick and the 3′ PEgRNA nick (the IND) without insertion of any exogenous sequence (a “clean deletion”). Alternatively, the 5′ editing template and the 3′ editing template can further comprise a region of complementarity to each other, and the region of complementarity can replace the IND in the DMPK gene. For example, in some embodiments, the 5′ PEgRNA is part of a dual prime editing composition that further comprises a 3′ PEgRNA, wherein the 5′ PEgRNA comprises a RTT comprising a sequence selected from sequence numbers 3459-3461 and 3467-3471, and the 3′ PEgRNA comprises a spacer comprising nucleotides 5-20 of sequence number 1525. In some embodiments, the 5′ PEgRNA is part of a dual prime editing composition that further comprises a 3′ PEgRNA, wherein the 5′ PEgRNA comprises a RTT comprising a sequence selected from sequence numbers 3462-3466, and the 3′ PEgRNA comprises a spacer comprising nucleotides 5-20 of sequence number 2353. In some embodiments, the 5′ PEgRNA is part of a dual prime editing composition that further comprises a 3′ PEgRNA, wherein the 5′ PEgRNA comprises a RTT comprising a sequence selected from sequence numbers 3472-3476, and the 3′ PEgRNA comprises a spacer comprising nucleotides 5-20 of sequence number 1936. In some embodiments, the 5′ PEgRNA is part of a dual prime editing composition that further comprises a 3′ PEgRNA, wherein the 5′ PEgRNA comprises a RTT comprising nucleotides x-40 of sequence number 3647, 3648, 3649, 3650, 3651, 3652, 3653, 3654, 3655, 3656, 3657, 3658, 3659, 3660, 3661, 3662, or 3663, wherein the 3′ PEgRNA comprises a spacer comprising nucleotides 5-20 of sequence number 1900, 1918, 1403, 1936, 2263, 2353, 1503, 2281, 2673, 1525, 2299, 2317, 1864, 1359, 2335, 1882, or 1381 respectively in the same order, and wherein x is any integer from 1 to 31. Preferably, the 3′ PEgRNA comprises a “clean deletion” RTT, for example, an RTT selected from Table 40 that has a Pairing 5′ spacer number that is 10. Each 5′ PEgRNA is assigned a RTT pairing number, which corresponds to a pairing 3′ spacer number in Table 39. The 5′ PEgRNA can be used in a dual prime editing system or composition with a 3′ PEgRNA comprising a spacer of the same pairing 3′ spacer number.

### Tables 11A and 11B

Tables 11A and 11B provide exemplary 5′ PEgRNAs and components that can be used with any prime editor containing a Cas9 protein capable of recognizing a NGG PAM sequence, wherein N is A, G, C, or T. The PEgRNAs exemplified in Tables 11A and 11B comprise: (a) a spacer comprising at its 3′ end a sequence corresponding to nucleotides 5-20 of sequence number 590, (b) a gRNA core capable of binding to a Cas9 protein, and (c) an extension arm comprising (i) an editing template and (ii) a primer binding site (PBS) comprising at its 5′ end the sequence corresponding to sequence number 605.

The PEgRNA spacer can be, for example, 16-22 nucleotides in length. In some embodiments, the PEgNA spacer is 16-20 nucleotides in length. In some embodiments, the PEgRNA spacer comprises nucleotides 5-20, 4-20, 3-20, 2-20, or 1-20 of sequence number 590. In some embodiments, the PEgRNA spacer comprises the sequence of sequence number 590. In some embodiments, the PEgRNA spacer is 20 nucleotides in length. Any of these spacers exemplified can be referred to as 5′ spacer No. 11.

The PBS can be, for example, 3 to 19 nucleotides in length. In some embodiments, the PBS is 3 to 17 nucleotides in length and corresponds to a PBS sequence in Table 11A. In some cases, a PBS length of no more than 3 nucleotides less than the PEgRNA spacer length is chosen.

The 5′ PEgRNAs of Tables 11A and 11B can be used in a dual prime editing system or composition further comprising any suitable 3′ PEgRNA. The prime editing system can be used, e.g., for editing a DMPK gene harboring 50 or more CTG repeats in the DMPK gene.

In some embodiments, a prime editing composition comprises a 5′ PEgRNA or component thereof exemplified in Tables 11A and 11B and a 3′ PEgRNA, wherein the editing template of the 5′ PEgRNA and the 3′ PEgRNA each comprises a region of complementarity to each other. For example, the editing template of the 5′ PEgRNA can comprise any of the RTT sequences provided in Table 37. The terms editing template and RTT may be used interchangeably. Each of the RTT sequences in Table 37 is assigned a RTT pairing number that reflects the complementarity relationship between a 5′ RTT and a 3′ RTT. The 5′ PEgRNA can be used in a dual prime editing system that further comprises a 3′ PEgRNA, e.g., a 3′ PEgRNA having a spacer sequence and a corresponding PBS as exemplified in Tables 20A-36B, wherein the 3′ PEgRNA comprises an editing template comprising a RTT sequence having a region of complementarity to the 5′ RTT. The 3′ RTT can comprise any of the RTT sequences provided in Table 38, wherein the 3′ RTT sequence has the same RTT Pairing number as that of the 5′ RTT. The editing template of the 5′ PEgRNA and the 3′ PEgRNA can be completely complementary to each other, wherein the 5′ editing template and the 3′ editing template have the same length and perfect complementarity to each other. Alternatively, the 5′ editing template can comprise a region of complementarity to the 3′ editing template, and further comprise a non-complementary region to the 3′ editing template. The 3′ editing template can comprise a region of complementarity to the 5′ editing template, and further comprise a non-complementary region to the 5′ editing template. The non-complementary region can be at the 5′ end or the 3′ end of the editing templates. Contacting the target DMPK gene with the prime editing system can result in deletion of the sequence between the 5′ PEgRNA nick and the 3′ PEgRNA nick (the IND) and insertion of the region of complementarity between the 5′ editing template and the 3′ editing template (the OD), or insertion of the region of complementarity between the 5′ editing template and the 3′ editing template as well as the non-complementarity regions (the RD), and hence replace the CTG expansion with the sequence of the OD or the RD. Each of the 5′ PEgRNA is assigned a RTT pairing number, and can be used in a dual prime editing composition with a 3′ PEgRNA comprising an RTT having the same RTT pairing number.

In some embodiments, a prime editing composition comprises a 5′ PEgRNA or component thereof exemplified in Tables 11A and 11B and a 3′ PEgRNA, wherein the editing template of the 5′ PEgRNA comprises a region of identity to the spacer of the 3′ PEgRNA. In some embodiments, the editing template of the 3′ PEgRNA comprises a region of identity to the spacer of the 5′ PEgRNA. In some embodiments, the 5′ editing template comprises a region of complementarity to a region on the second strand of the DMPK gene that is directly 3′ to nucleotide 3 of the search target sequence of the 3′ PEgRNA. In some embodiments, the 3′ editing template comprises a region of complementarity to a region on the first strand of the DMPK gene that is directly 3′ to nucleotide 3 of the search target sequence of the 5′ PEgRNA. For example, the extension arm of the 5′ PEgRNA can comprise an editing template comprising the sequence of any of the RTT sequences provided in Table 39. Each of the RTT sequences in Table 39 is assigned a pairing 3′ spacer number. The 5′ PEgRNA can be used in a dual prime editing system with a 3′ PEgRNA, wherein the 3′ PEgRNA comprises a spacer that has the Pairing 3′ spacer number assigned to the 5′ RTT in Table 39. Contacting the target DMPK gene with the prime editing system can result in deletion of the sequence between the 5′ PEgRNA nick and the 3′ PEgRNA nick (the IND) without insertion of any exogenous sequence (a “clean deletion”). Alternatively, the 5′ editing template and the 3′ editing template can further comprise a region of complementarity to each other, and the region of complementarity can replace the IND in the DMPK gene. For example, in some embodiments, the 5′ PEgRNA is part of a dual prime editing composition that further comprises a 3′ PEgRNA, wherein the 5′ PEgRNA comprises a RTT comprising a sequence selected from sequence numbers 3459-3461 and 3467-3471, and the 3′ PEgRNA comprises a spacer comprising nucleotides 5-20 of sequence number 1525. In some embodiments, the 5′ PEgRNA is part of a dual prime editing composition that further comprises a 3′ PEgRNA, wherein the 5′ PEgRNA comprises a RTT comprising a sequence selected from sequence numbers 3462-3466, and the 3′ PEgRNA comprises a spacer comprising nucleotides 5-20 of sequence number 2353. In some embodiments, the 5′ PEgRNA is part of a dual prime editing composition that further comprises a 3′ PEgRNA, wherein the 5′ PEgRNA comprises a RTT comprising a sequence selected from sequence numbers 3472-3476, and the 3′ PEgRNA comprises a spacer comprising nucleotides 5-20 of sequence number 1936. In some embodiments, the 5′ PEgRNA is part of a dual prime editing composition that further comprises a 3′ PEgRNA, wherein the 5′ PEgRNA comprises a RTT comprising nucleotides x-40 of sequence number 3647, 3648, 3649, 3650, 3651, 3652, 3653, 3654, 3655, 3656, 3657, 3658, 3659, 3660, 3661, 3662, or 3663, wherein the 3′ PEgRNA comprises a spacer comprising nucleotides 5-20 of sequence number 1900, 1918, 1403, 1936, 2263, 2353, 1503, 2281, 2673, 1525, 2299, 2317, 1864, 1359, 2335, 1882, or 1381 respectively in the same order, and wherein x is any integer from 1 to 31. Preferably, the 3′ PEgRNA comprises a “clean deletion” RTT, for example, an RTT selected from Table 40 that has a Pairing 5′ spacer number that is 11. Each 5′ PEgRNA is assigned a RTT pairing number, which corresponds to a pairing 3′ spacer number in Table 39. The 5′ PEgRNA can be used in a dual prime editing system or composition with a 3′ PEgRNA comprising a spacer of the same pairing 3′ spacer number.

### Tables 12A and 12B

Tables 12A and 12B provide exemplary 5′ PEgRNAs and components that can be used with any prime editor containing a Cas9 protein capable of recognizing a NGG PAM sequence, wherein N is A, G, C, or T. The PEgRNAs exemplified in Tables 12A and 12B comprise: (a) a spacer comprising at its 3′ end a sequence corresponding to nucleotides 5-20 of sequence number 912, (b) a gRNA core capable of binding to a Cas9 protein, and (c) an extension arm comprising (i) an editing template and (ii) a primer binding site (PBS) comprising at its 5′ end the sequence corresponding to sequence number 927.

The PEgRNA spacer can be, for example, 16-22 nucleotides in length. In some embodiments, the PEgNA spacer is 16-20 nucleotides in length. In some embodiments, the PEgRNA spacer comprises nucleotides 5-20, 4-20, 3-20, 2-20, or 1-20 of sequence number 912. In some embodiments, the PEgRNA spacer comprises the sequence of sequence number 912. In some embodiments, the PEgRNA spacer is 20 nucleotides in length. Any of these spacers exemplified can be referred to as 5′ spacer No. 12.

The PBS can be, for example, 3 to 19 nucleotides in length. In some embodiments, the PBS is 3 to 17 nucleotides in length and corresponds to a PBS sequence in Table 12A. In some cases, a PBS length of no more than 3 nucleotides less than the PEgRNA spacer length is chosen.

The 5′ PEgRNAs of Tables 12A and 12B can be used in a dual prime editing system or composition further comprising any suitable 3′ PEgRNA. The prime editing system can be used, e.g., for editing a DMPK gene harboring 50 or more CTG repeats in the DMPK gene.

In some embodiments, a prime editing composition comprises a 5′ PEgRNA or component thereof exemplified in Tables 12A and 12B and a 3′ PEgRNA, wherein the editing template of the 5′ PEgRNA and the 3′ PEgRNA each comprises a region of complementarity to each other. For example, the editing template of the 5′ PEgRNA can comprise any of the RTT sequences provided in Table 37. The terms editing template and RTT may be used interchangeably. Each of the RTT sequences in Table 37 is assigned a RTT pairing number that reflects the complementarity relationship between a 5′ RTT and a 3′ RTT. The 5′ PEgRNA can be used in a dual prime editing system that further comprises a 3′ PEgRNA, e.g., a 3′ PEgRNA having a spacer sequence and a corresponding PBS as exemplified in Tables 20A-36B, wherein the 3′ PEgRNA comprises an editing template comprising a RTT sequence having a region of complementarity to the 5′ RTT. The 3′ RTT can comprise any of the RTT sequences provided in Table 38, wherein the 3′ RTT sequence has the same RTT Pairing number as that of the 5′ RTT. The editing template of the 5′ PEgRNA and the 3′ PEgRNA can be completely complementary to each other, wherein the 5′ editing template and the 3′ editing template have the same length and perfect complementarity to each other. Alternatively, the 5′ editing template can comprise a region of complementarity to the 3′ editing template, and further comprise a non-complementary region to the 3′ editing template. The 3′ editing template can comprise a region of complementarity to the 5′ editing template, and further comprise a non-complementary region to the 5′ editing template. The non-complementary region can be at the 5′ end or the 3′ end of the editing templates. Contacting the target DMPK gene with the prime editing system can result in deletion of the sequence between the 5′ PEgRNA nick and the 3′ PEgRNA nick (the IND) and insertion of the region of complementarity between the 5′ editing template and the 3′ editing template (the OD), or insertion of the region of complementarity between the 5′ editing template and the 3′ editing template as well as the non-complementarity regions (the RD), and hence replace the CTG expansion with the sequence of the OD or the RD. Each of the 5′ PEgRNA is assigned a RTT pairing number, and can be used in a dual prime editing composition with a 3′ PEgRNA comprising an RTT having the same RTT pairing number.

In some embodiments, a prime editing composition comprises a 5′ PEgRNA or component thereof exemplified in Tables 12A and 12B and a 3′ PEgRNA, wherein the editing template of the 5′ PEgRNA comprises a region of identity to the spacer of the 3′ PEgRNA. In some embodiments, the editing template of the 3′ PEgRNA comprises a region of identity to the spacer of the 5′ PEgRNA. In some embodiments, the 5′ editing template comprises a region of complementarity to a region on the second strand of the DMPK gene that is directly 3′ to nucleotide 3 of the search target sequence of the 3′ PEgRNA. In some embodiments, the 3′ editing template comprises a region of complementarity to a region on the first strand of the DMPK gene that is directly 3′ to nucleotide 3 of the search target sequence of the 5′ PEgRNA. For example, the extension arm of the 5′ PEgRNA can comprise an editing template comprising the sequence of any of the RTT sequences provided in Table 39. Each of the RTT sequences in Table 39 is assigned a pairing 3′ spacer number. The 5′ PEgRNA can be used in a dual prime editing system with a 3′ PEgRNA, wherein the 3′ PEgRNA comprises a spacer that has the Pairing 3′ spacer number assigned to the 5′ RTT in Table 39. Contacting the target DMPK gene with the prime editing system can result in deletion of the sequence between the 5′ PEgRNA nick and the 3′ PEgRNA nick (the IND) without insertion of any exogenous sequence (a “clean deletion”). Alternatively, the 5′ editing template and the 3′ editing template can further comprise a region of complementarity to each other, and the region of complementarity can replace the IND in the DMPK gene. For example, in some embodiments, the 5′ PEgRNA is part of a dual prime editing composition that further comprises a 3′ PEgRNA, wherein the 5′ PEgRNA comprises a RTT comprising a sequence selected from sequence numbers 3459-3461 and 3467-3471, and the 3′ PEgRNA comprises a spacer comprising nucleotides 5-20 of sequence number 1525. In some embodiments, the 5′ PEgRNA is part of a dual prime editing composition that further comprises a 3′ PEgRNA, wherein the 5′ PEgRNA comprises a RTT comprising a sequence selected from sequence numbers 3462-3466, and the 3′ PEgRNA comprises a spacer comprising nucleotides 5-20 of sequence number 2353. In some embodiments, the 5′ PEgRNA is part of a dual prime editing composition that further comprises a 3′ PEgRNA, wherein the 5′ PEgRNA comprises a RTT comprising a sequence selected from sequence numbers 3472-3476, and the 3′ PEgRNA comprises a spacer comprising nucleotides 5-20 of sequence number 1936. In some embodiments, the 5′ PEgRNA is part of a dual prime editing composition that further comprises a 3′ PEgRNA, wherein the 5′ PEgRNA comprises a RTT comprising nucleotides x-40 of sequence number 3647, 3648, 3649, 3650, 3651, 3652, 3653, 3654, 3655, 3656, 3657, 3658, 3659, 3660, 3661, 3662, or 3663, wherein the 3′ PEgRNA comprises a spacer comprising nucleotides 5-20 of sequence number 1900, 1918, 1403, 1936, 2263, 2353, 1503, 2281, 2673, 1525, 2299, 2317, 1864, 1359, 2335, 1882, or 1381 respectively in the same order, and wherein x is any integer from 1 to 31. Preferably, the 3′ PEgRNA comprises a “clean deletion” RTT, for example, an RTT selected from Table 40 that has a Pairing 5′ spacer number that is 12. Each 5′ PEgRNA is assigned a RTT pairing number, which corresponds to a pairing 3′ spacer number in Table 39. The 5′ PEgRNA can be used in a dual prime editing system or composition with a 3′ PEgRNA comprising a spacer of the same pairing 3′ spacer number.

### Tables 13A and 13B

Tables 13A and 13B provide exemplary 5′ PEgRNAs and components that can be used with any prime editor containing a Cas9 protein capable of recognizing a NGG PAM sequence, wherein N is A, G, C, or T. The PEgRNAs exemplified in Tables 13A and 13B comprise: (a) a spacer comprising at its 3′ end a sequence corresponding to nucleotides 5-20 of sequence number 929, (b) a gRNA core capable of binding to a Cas9 protein, and (c) an extension arm comprising (i) an editing template and (ii) a primer binding site (PBS) comprising at its 5′ end the sequence corresponding to sequence number 944.

The PEgRNA spacer can be, for example, 16-22 nucleotides in length. In some embodiments, the PEgNA spacer is 16-20 nucleotides in length. In some embodiments, the PEgRNA spacer comprises nucleotides 5-20, 4-20, 3-20, 2-20, or 1-20 of sequence number 929. In some embodiments, the PEgRNA spacer comprises the sequence of sequence number 929. In some embodiments, the PEgRNA spacer is 20 nucleotides in length. Any of these spacers exemplified can be referred to as 5′ spacer No. 13.

The PBS can be, for example, 3 to 19 nucleotides in length. In some embodiments, the PBS is 3 to 17 nucleotides in length and corresponds to a PBS sequence in Table 13A. In some cases, a PBS length of no more than 3 nucleotides less than the PEgRNA spacer length is chosen.

The 5′ PEgRNAs of Tables 13A and 13B can be used in a dual prime editing system or composition further comprising any suitable 3′ PEgRNA. The prime editing system can be used, e.g., for editing a DMPK gene harboring 50 or more CTG repeats in the DMPK gene.

In some embodiments, a prime editing composition comprises a 5′ PEgRNA or component thereof exemplified in Tables 13A and 13B and a 3′ PEgRNA, wherein the editing template of the 5′ PEgRNA and the 3′ PEgRNA each comprises a region of complementarity to each other. For example, the editing template of the 5′ PEgRNA can comprise any of the RTT sequences provided in Table 37. The terms editing template and RTT may be used interchangeably. Each of the RTT sequences in Table 37 is assigned a RTT pairing number that reflects the complementarity relationship between a 5′ RTT and a 3′ RTT. The 5′ PEgRNA can be used in a dual prime editing system that further comprises a 3′ PEgRNA, e.g., a 3′ PEgRNA having a spacer sequence and a corresponding PBS as exemplified in Tables 20A-36B, wherein the 3′ PEgRNA comprises an editing template comprising a RTT sequence having a region of complementarity to the 5′ RTT. The 3′ RTT can comprise any of the RTT sequences provided in Table 38, wherein the 3′ RTT sequence has the same RTT Pairing number as that of the 5′ RTT. The editing template of the 5′ PEgRNA and the 3′ PEgRNA can be completely complementary to each other, wherein the 5′ editing template and the 3′ editing template have the same length and perfect complementarity to each other. Alternatively, the 5′ editing template can comprise a region of complementarity to the 3′ editing template, and further comprise a non-complementary region to the 3′ editing template. The 3′ editing template can comprise a region of complementarity to the 5′ editing template, and further comprise a non-complementary region to the 5′ editing template. The non-complementary region can be at the 5′ end or the 3′ end of the editing templates. Contacting the target DMPK gene with the prime editing system can result in deletion of the sequence between the 5′ PEgRNA nick and the 3′ PEgRNA nick (the IND) and insertion of the region of complementarity between the 5′ editing template and the 3′ editing template (the OD), or insertion of the region of complementarity between the 5′ editing template and the 3′ editing template as well as the non-complementarity regions (the RD), and hence replace the CTG expansion with the sequence of the OD or the RD. Each of the 5′ PEgRNA is assigned a RTT pairing number, and can be used in a dual prime editing composition with a 3′ PEgRNA comprising an RTT having the same RTT pairing number.

In some embodiments, a prime editing composition comprises a 5′ PEgRNA or component thereof exemplified in Tables 13A and 13B and a 3′ PEgRNA, wherein the editing template of the 5′ PEgRNA comprises a region of identity to the spacer of the 3′ PEgRNA. In some embodiments, the editing template of the 3′ PEgRNA comprises a region of identity to the spacer of the 5′ PEgRNA. In some embodiments, the 5′ editing template comprises a region of complementarity to a region on the second strand of the DMPK gene that is directly 3′ to nucleotide 3 of the search target sequence of the 3′ PEgRNA. In some embodiments, the 3′ editing template comprises a region of complementarity to a region on the first strand of the DMPK gene that is directly 3′ to nucleotide 3 of the search target sequence of the 5′ PEgRNA. For example, the extension arm of the 5′ PEgRNA can comprise an editing template comprising the sequence of any of the RTT sequences provided in Table 39. Each of the RTT sequences in Table 39 is assigned a pairing 3′ spacer number. The 5′ PEgRNA can be used in a dual prime editing system with a 3′ PEgRNA, wherein the 3′ PEgRNA comprises a spacer that has the Pairing 3′ spacer number assigned to the 5′ RTT in Table 39. Contacting the target DMPK gene with the prime editing system can result in deletion of the sequence between the 5′ PEgRNA nick and the 3′ PEgRNA nick (the IND) without insertion of any exogenous sequence (a “clean deletion”). Alternatively, the 5′ editing template and the 3′ editing template can further comprise a region of complementarity to each other, and the region of complementarity can replace the IND in the DMPK gene. For example, in some embodiments, the 5′ PEgRNA is part of a dual prime editing composition that further comprises a 3′ PEgRNA, wherein the 5′ PEgRNA comprises a RTT comprising a sequence selected from sequence numbers 3459-3461 and 3467-3471, and the 3′ PEgRNA comprises a spacer comprising nucleotides 5-20 of sequence number 1525. In some embodiments, the 5′ PEgRNA is part of a dual prime editing composition that further comprises a 3′ PEgRNA, wherein the 5′ PEgRNA comprises a RTT comprising a sequence selected from sequence numbers 3462-3466, and the 3′ PEgRNA comprises a spacer comprising nucleotides 5-20 of sequence number 2353. In some embodiments, the 5′ PEgRNA is part of a dual prime editing composition that further comprises a 3′ PEgRNA, wherein the 5′ PEgRNA comprises a RTT comprising a sequence selected from sequence numbers 3472-3476, and the 3′ PEgRNA comprises a spacer comprising nucleotides 5-20 of sequence number 1936. In some embodiments, the 5′ PEgRNA is part of a dual prime editing composition that further comprises a 3′ PEgRNA, wherein the 5′ PEgRNA comprises a RTT comprising nucleotides x-40 of sequence number 3647, 3648, 3649, 3650, 3651, 3652, 3653, 3654, 3655, 3656, 3657, 3658, 3659, 3660, 3661, 3662, or 3663, wherein the 3′ PEgRNA comprises a spacer comprising nucleotides 5-20 of sequence number 1900, 1918, 1403, 1936, 2263, 2353, 1503, 2281, 2673, 1525, 2299, 2317, 1864, 1359, 2335, 1882, or 1381 respectively in the same order, and wherein x is any integer from 1 to 31. Preferably, the 3′ PEgRNA comprises a “clean deletion” RTT, for example, an RTT selected from Table 40 that has a Pairing 5′ spacer number that is 13. Each 5′ PEgRNA is assigned a RTT pairing number, which corresponds to a pairing 3′ spacer number in Table 39. The 5′ PEgRNA can be used in a dual prime editing system or composition with a 3′ PEgRNA comprising a spacer of the same pairing 3′ spacer number.

### Tables 14A and 14B

Tables 14A and 14B provide exemplary 5′ PEgRNAs and components that can be used with any prime editor containing a Cas9 protein capable of recognizing a NGG PAM sequence, wherein N is A, G, C, or T. The PEgRNAs exemplified in Tables 14A and 14B comprise: (a) a spacer comprising at its 3′ end a sequence corresponding to nucleotides 5-20 of sequence number 947, (b) a gRNA core capable of binding to a Cas9 protein, and (c) an extension arm comprising (i) an editing template and (ii) a primer binding site (PBS) comprising at its 5′ end the sequence corresponding to sequence number 962.

The PEgRNA spacer can be, for example, 16-22 nucleotides in length. In some embodiments, the PEgNA spacer is 16-20 nucleotides in length. In some embodiments, the PEgRNA spacer comprises nucleotides 5-20, 4-20, 3-20, 2-20, or 1-20 of sequence number 947. In some embodiments, the PEgRNA spacer comprises the sequence of sequence number 947. In some embodiments, the PEgRNA spacer is 20 nucleotides in length. Any of these spacers exemplified can be referred to as 5′ spacer No. 14.

The PBS can be, for example, 3 to 19 nucleotides in length. In some embodiments, the PBS is 3 to 17 nucleotides in length and corresponds to a PBS sequence in Table 14A. In some cases, a PBS length of no more than 3 nucleotides less than the PEgRNA spacer length is chosen.

The 5′ PEgRNAs of Tables 14A and 14B can be used in a dual prime editing system or composition further comprising any suitable 3′ PEgRNA. The prime editing system can be used, e.g., for editing a DMPK gene harboring 50 or more CTG repeats in the DMPK gene.

In some embodiments, a prime editing composition comprises a 5′ PEgRNA or component thereof exemplified in Tables 14A and 14B and a 3′ PEgRNA, wherein the editing template of the 5′ PEgRNA and the 3′ PEgRNA each comprises a region of complementarity to each other. For example, the editing template of the 5′ PEgRNA can comprise any of the RTT sequences provided in Table 37. The terms editing template and RTT may be used interchangeably. Each of the RTT sequences in Table 37 is assigned a RTT pairing number that reflects the complementarity relationship between a 5′ RTT and a 3′ RTT. The 5′ PEgRNA can be used in a dual prime editing system that further comprises a 3′ PEgRNA, e.g., a 3′ PEgRNA having a spacer sequence and a corresponding PBS as exemplified in Tables 20A-36B, wherein the 3′ PEgRNA comprises an editing template comprising a RTT sequence having a region of complementarity to the 5′ RTT. The 3′ RTT can comprise any of the RTT sequences provided in Table 38, wherein the 3′ RTT sequence has the same RTT Pairing number as that of the 5′ RTT. The editing template of the 5′ PEgRNA and the 3′ PEgRNA can be completely complementary to each other, wherein the 5′ editing template and the 3′ editing template have the same length and perfect complementarity to each other. Alternatively, the 5′ editing template can comprise a region of complementarity to the 3′ editing template, and further comprise a non-complementary region to the 3′ editing template. The 3′ editing template can comprise a region of complementarity to the 5′ editing template, and further comprise a non-complementary region to the 5′ editing template. The non-complementary region can be at the 5′ end or the 3′ end of the editing templates. Contacting the target DMPK gene with the prime editing system can result in deletion of the sequence between the 5′ PEgRNA nick and the 3′ PEgRNA nick (the IND) and insertion of the region of complementarity between the 5′ editing template and the 3′ editing template (the OD), or insertion of the region of complementarity between the 5′ editing template and the 3′ editing template as well as the non-complementarity regions (the RD), and hence replace the CTG expansion with the sequence of the OD or the RD. Each of the 5′ PEgRNA is assigned a RTT pairing number, and can be used in a dual prime editing composition with a 3′ PEgRNA comprising an RTT having the same RTT pairing number.

In some embodiments, a prime editing composition comprises a 5′ PEgRNA or component thereof exemplified in Tables 14A and 14B and a 3′ PEgRNA, wherein the editing template of the 5′ PEgRNA comprises a region of identity to the spacer of the 3′ PEgRNA. In some embodiments, the editing template of the 3′ PEgRNA comprises a region of identity to the spacer of the 5′ PEgRNA. In some embodiments, the 5′ editing template comprises a region of complementarity to a region on the second strand of the DMPK gene that is directly 3′ to nucleotide 3 of the search target sequence of the 3′ PEgRNA. In some embodiments, the 3′ editing template comprises a region of complementarity to a region on the first strand of the DMPK gene that is directly 3′ to nucleotide 3 of the search target sequence of the 5′ PEgRNA. For example, the extension arm of the 5′ PEgRNA can comprise an editing template comprising the sequence of any of the RTT sequences provided in Table 39. Each of the RTT sequences in Table 39 is assigned a pairing 3′ spacer number. The 5′ PEgRNA can be used in a dual prime editing system with a 3′ PEgRNA, wherein the 3′ PEgRNA comprises a spacer that has the Pairing 3′ spacer number assigned to the 5′ RTT in Table 39. Contacting the target DMPK gene with the prime editing system can result in deletion of the sequence between the 5′ PEgRNA nick and the 3′ PEgRNA nick (the IND) without insertion of any exogenous sequence (a “clean deletion”). Alternatively, the 5′ editing template and the 3′ editing template can further comprise a region of complementarity to each other, and the region of complementarity can replace the IND in the DMPK gene. For example, in some embodiments, the 5′ PEgRNA is part of a dual prime editing composition that further comprises a 3′ PEgRNA, wherein the 5′ PEgRNA comprises a RTT comprising a sequence selected from sequence numbers 3459-3461 and 3467-3471, and the 3′ PEgRNA comprises a spacer comprising nucleotides 5-20 of sequence number 1525. In some embodiments, the 5′ PEgRNA is part of a dual prime editing composition that further comprises a 3′ PEgRNA, wherein the 5′ PEgRNA comprises a RTT comprising a sequence selected from sequence numbers 3462-3466, and the 3′ PEgRNA comprises a spacer comprising nucleotides 5-20 of sequence number 2353. In some embodiments, the 5′ PEgRNA is part of a dual prime editing composition that further comprises a 3′ PEgRNA, wherein the 5′ PEgRNA comprises a RTT comprising a sequence selected from sequence numbers 3472-3476, and the 3′ PEgRNA comprises a spacer comprising nucleotides 5-20 of sequence number 1936. In some embodiments, the 5′ PEgRNA is part of a dual prime editing composition that further comprises a 3′ PEgRNA, wherein the 5′ PEgRNA comprises a RTT comprising nucleotides x-40 of sequence number 3647, 3648, 3649, 3650, 3651, 3652, 3653, 3654, 3655, 3656, 3657, 3658, 3659, 3660, 3661, 3662, or 3663, wherein the 3′ PEgRNA comprises a spacer comprising nucleotides 5-20 of sequence number 1900, 1918, 1403, 1936, 2263, 2353, 1503, 2281, 2673, 1525, 2299, 2317, 1864, 1359, 2335, 1882, or 1381 respectively in the same order, and wherein x is any integer from 1 to 31. Preferably, the 3′ PEgRNA comprises a “clean deletion” RTT, for example, an RTT selected from Table 40 that has a Pairing 5′ spacer number that is 14. Each 5′ PEgRNA is assigned a RTT pairing number, which corresponds to a pairing 3′ spacer number in Table 39. The 5′ PEgRNA can be used in a dual prime editing system or composition with a 3′ PEgRNA comprising a spacer of the same pairing 3′ spacer number.

### Tables 15A and 15B

Tables 15A and 15B provide exemplary 5′ PEgRNAs and components that can be used with any prime editor containing a Cas9 protein capable of recognizing a NGG PAM sequence, wherein N is A, G, C, or T. The PEgRNAs exemplified in Tables 15A and 15B comprise: (a) a spacer comprising at its 3′ end a sequence corresponding to nucleotides 5-20 of sequence number 965, (b) a gRNA core capable of binding to a Cas9 protein, and (c) an extension arm comprising (i) an editing template and (ii) a primer binding site (PBS) comprising at its 5′ end the sequence corresponding to sequence number 980.

The PEgRNA spacer can be, for example, 16-22 nucleotides in length. In some embodiments, the PEgNA spacer is 16-20 nucleotides in length. In some embodiments, the PEgRNA spacer comprises nucleotides 5-20, 4-20, 3-20, 2-20, or 1-20 of sequence number 965. In some embodiments, the PEgRNA spacer comprises the sequence of sequence number 965. In some embodiments, the PEgRNA spacer is 20 nucleotides in length. Any of these spacers exemplified can be referred to as 5′ spacer No. 15.

The PBS can be, for example, 3 to 19 nucleotides in length. In some embodiments, the PBS is 3 to 17 nucleotides in length and corresponds to a PBS sequence in Table 15A. In some cases, a PBS length of no more than 3 nucleotides less than the PEgRNA spacer length is chosen.

The 5′ PEgRNAs of Tables 15A and 15B can be used in a dual prime editing system or composition further comprising any suitable 3′ PEgRNA. The prime editing system can be used, e.g., for editing a DMPK gene harboring 50 or more CTG repeats in the DMPK gene.

In some embodiments, a prime editing composition comprises a 5′ PEgRNA or component thereof exemplified in Tables 15A and 15B and a 3′ PEgRNA, wherein the editing template of the 5′ PEgRNA and the 3′ PEgRNA each comprises a region of complementarity to each other. For example, the editing template of the 5′ PEgRNA can comprise any of the RTT sequences provided in Table 37. The terms editing template and RTT may be used interchangeably. Each of the RTT sequences in Table 37 is assigned a RTT pairing number that reflects the complementarity relationship between a 5′ RTT and a 3′ RTT. The 5′ PEgRNA can be used in a dual prime editing system that further comprises a 3′ PEgRNA, e.g., a 3′ PEgRNA having a spacer sequence and a corresponding PBS as exemplified in Tables 20A-36B, wherein the 3′ PEgRNA comprises an editing template comprising a RTT sequence having a region of complementarity to the 5′ RTT. The 3′ RTT can comprise any of the RTT sequences provided in Table 38, wherein the 3′ RTT sequence has the same RTT Pairing number as that of the 5′ RTT. The editing template of the 5′ PEgRNA and the 3′ PEgRNA can be completely complementary to each other, wherein the 5′ editing template and the 3′ editing template have the same length and perfect complementarity to each other. Alternatively, the 5′ editing template can comprise a region of complementarity to the 3′ editing template, and further comprise a non-complementary region to the 3′ editing template. The 3′ editing template can comprise a region of complementarity to the 5′ editing template, and further comprise a non-complementary region to the 5′ editing template. The non-complementary region can be at the 5′ end or the 3′ end of the editing templates. Contacting the target DMPK gene with the prime editing system can result in deletion of the sequence between the 5′ PEgRNA nick and the 3′ PEgRNA nick (the IND) and insertion of the region of complementarity between the 5′ editing template and the 3′ editing template (the OD), or insertion of the region of complementarity between the 5′ editing template and the 3′ editing template as well as the non-complementarity regions (the RD), and hence replace the CTG expansion with the sequence of the OD or the RD. Each of the 5′ PEgRNA is assigned a RTT pairing number, and can be used in a dual prime editing composition with a 3′ PEgRNA comprising an RTT having the same RTT pairing number.

In some embodiments, a prime editing composition comprises a 5′ PEgRNA or component thereof exemplified in Tables 15A and 15B and a 3′ PEgRNA, wherein the editing template of the 5′ PEgRNA comprises a region of identity to the spacer of the 3′ PEgRNA. In some embodiments, the editing template of the 3′ PEgRNA comprises a region of identity to the spacer of the 5′ PEgRNA. In some embodiments, the 5′ editing template comprises a region of complementarity to a region on the second strand of the DMPK gene that is directly 3′ to nucleotide 3 of the search target sequence of the 3′ PEgRNA. In some embodiments, the 3′ editing template comprises a region of complementarity to a region on the first strand of the DMPK gene that is directly 3′ to nucleotide 3 of the search target sequence of the 5′ PEgRNA. For example, the extension arm of the 5′ PEgRNA can comprise an editing template comprising the sequence of any of the RTT sequences provided in Table 39. Each of the RTT sequences in Table 39 is assigned a pairing 3′ spacer number. The 5′ PEgRNA can be used in a dual prime editing system with a 3′ PEgRNA, wherein the 3′ PEgRNA comprises a spacer that has the Pairing 3′ spacer number assigned to the 5′ RTT in Table 39. Contacting the target DMPK gene with the prime editing system can result in deletion of the sequence between the 5′ PEgRNA nick and the 3′ PEgRNA nick (the IND) without insertion of any exogenous sequence (a “clean deletion”). Alternatively, the 5′ editing template and the 3′ editing template can further comprise a region of complementarity to each other, and the region of complementarity can replace the IND in the DMPK gene. For example, in some embodiments, the 5′ PEgRNA is part of a dual prime editing composition that further comprises a 3′ PEgRNA, wherein the 5′ PEgRNA comprises a RTT comprising a sequence selected from sequence numbers 3459-3461 and 3467-3471, and the 3′ PEgRNA comprises a spacer comprising nucleotides 5-20 of sequence number 1525. In some embodiments, the 5′ PEgRNA is part of a dual prime editing composition that further comprises a 3′ PEgRNA, wherein the 5′ PEgRNA comprises a RTT comprising a sequence selected from sequence numbers 3462-3466, and the 3′ PEgRNA comprises a spacer comprising nucleotides 5-20 of sequence number 2353. In some embodiments, the 5′ PEgRNA is part of a dual prime editing composition that further comprises a 3′ PEgRNA, wherein the 5′ PEgRNA comprises a RTT comprising a sequence selected from sequence numbers 3472-3476, and the 3′ PEgRNA comprises a spacer comprising nucleotides 5-20 of sequence number 1936. In some embodiments, the 5′ PEgRNA is part of a dual prime editing composition that further comprises a 3′ PEgRNA, wherein the 5′ PEgRNA comprises a RTT comprising nucleotides x-40 of sequence number 3647, 3648, 3649, 3650, 3651, 3652, 3653, 3654, 3655, 3656, 3657, 3658, 3659, 3660, 3661, 3662, or 3663, wherein the 3′ PEgRNA comprises a spacer comprising nucleotides 5-20 of sequence number 1900, 1918, 1403, 1936, 2263, 2353, 1503, 2281, 2673, 1525, 2299, 2317, 1864, 1359, 2335, 1882, or 1381 respectively in the same order, and wherein x is any integer from 1 to 31. Preferably, the 3′ PEgRNA comprises a “clean deletion” RTT, for example, an RTT selected from Table 40 that has a Pairing 5′ spacer number that is 15. Each 5′ PEgRNA is assigned a RTT pairing number, which corresponds to a pairing 3′ spacer number in Table 39. The 5′ PEgRNA can be used in a dual prime editing system or composition with a 3′ PEgRNA comprising a spacer of the same pairing 3′ spacer number.

### Tables 16A and 16B

Tables 16A and 16B provide exemplary 5′ PEgRNAs and components that can be used with any prime editor containing a Cas9 protein capable of recognizing a NGG PAM sequence, wherein N is A, G, C, or T. The PEgRNAs exemplified in Tables 16A and 16B comprise: (a) a spacer comprising at its 3′ end a sequence corresponding to nucleotides 5-20 of sequence number 983, (b) a gRNA core capable of binding to a Cas9 protein, and (c) an extension arm comprising (i) an editing template and (ii) a primer binding site (PBS) comprising at its 5′ end the sequence corresponding to sequence number 998.

The PEgRNA spacer can be, for example, 16-22 nucleotides in length. In some embodiments, the PEgNA spacer is 16-20 nucleotides in length. In some embodiments, the PEgRNA spacer comprises nucleotides 5-20, 4-20, 3-20, 2-20, or 1-20 of sequence number 983. In some embodiments, the PEgRNA spacer comprises the sequence of sequence number 983. In some embodiments, the PEgRNA spacer is 20 nucleotides in length. Any of these spacers exemplified can be referred to as 5′ spacer No. 16.

The PBS can be, for example, 3 to 19 nucleotides in length. In some embodiments, the PBS is 3 to 17 nucleotides in length and corresponds to a PBS sequence in Table 16A. In some cases, a PBS length of no more than 3 nucleotides less than the PEgRNA spacer length is chosen.

The 5′ PEgRNAs of Tables 16A and 16B can be used in a dual prime editing system or composition further comprising any suitable 3′ PEgRNA. The prime editing system can be used, e.g., for editing a DMPK gene harboring 50 or more CTG repeats in the DMPK gene.

In some embodiments, a prime editing composition comprises a 5′ PEgRNA or component thereof exemplified in Tables 16A and 16B and a 3′ PEgRNA, wherein the editing template of the 5′ PEgRNA and the 3′ PEgRNA each comprises a region of complementarity to each other. For example, the editing template of the 5′ PEgRNA can comprise any of the RTT sequences provided in Table 37. The terms editing template and RTT may be used interchangeably. Each of the RTT sequences in Table 37 is assigned a RTT pairing number that reflects the complementarity relationship between a 5′ RTT and a 3′ RTT. The 5′ PEgRNA can be used in a dual prime editing system that further comprises a 3′ PEgRNA, e.g., a 3′ PEgRNA having a spacer sequence and a corresponding PBS as exemplified in Tables 20A-36B, wherein the 3′ PEgRNA comprises an editing template comprising a RTT sequence having a region of complementarity to the 5′ RTT. The 3′ RTT can comprise any of the RTT sequences provided in Table 38, wherein the 3′ RTT sequence has the same RTT Pairing number as that of the 5′ RTT. The editing template of the 5′ PEgRNA and the 3′ PEgRNA can be completely complementary to each other, wherein the 5′ editing template and the 3′ editing template have the same length and perfect complementarity to each other. Alternatively, the 5′ editing template can comprise a region of complementarity to the 3′ editing template, and further comprise a non-complementary region to the 3′ editing template. The 3′ editing template can comprise a region of complementarity to the 5′ editing template, and further comprise a non-complementary region to the 5′ editing template. The non-complementary region can be at the 5′ end or the 3′ end of the editing templates. Contacting the target DMPK gene with the prime editing system can result in deletion of the sequence between the 5′ PEgRNA nick and the 3′ PEgRNA nick (the IND) and insertion of the region of complementarity between the 5′ editing template and the 3′ editing template (the OD), or insertion of the region of complementarity between the 5′ editing template and the 3′ editing template as well as the non-complementarity regions (the RD), and hence replace the CTG expansion with the sequence of the OD or the RD. Each of the 5′ PEgRNA is assigned a RTT pairing number, and can be used in a dual prime editing composition with a 3′ PEgRNA comprising an RTT having the same RTT pairing number.

In some embodiments, a prime editing composition comprises a 5′ PEgRNA or component thereof exemplified in Tables 16A and 16B and a 3′ PEgRNA, wherein the editing template of the 5′ PEgRNA comprises a region of identity to the spacer of the 3′ PEgRNA. In some embodiments, the editing template of the 3′ PEgRNA comprises a region of identity to the spacer of the 5′ PEgRNA. In some embodiments, the 5′ editing template comprises a region of complementarity to a region on the second strand of the DMPK gene that is directly 3′ to nucleotide 3 of the search target sequence of the 3′ PEgRNA. In some embodiments, the 3′ editing template comprises a region of complementarity to a region on the first strand of the DMPK gene that is directly 3′ to nucleotide 3 of the search target sequence of the 5′ PEgRNA. For example, the extension arm of the 5′ PEgRNA can comprise an editing template comprising the sequence of any of the RTT sequences provided in Table 39. Each of the RTT sequences in Table 39 is assigned a pairing 3′ spacer number. The 5′ PEgRNA can be used in a dual prime editing system with a 3′ PEgRNA, wherein the 3′ PEgRNA comprises a spacer that has the Pairing 3′ spacer number assigned to the 5′ RTT in Table 39. Contacting the target DMPK gene with the prime editing system can result in deletion of the sequence between the 5′ PEgRNA nick and the 3′ PEgRNA nick (the IND) without insertion of any exogenous sequence (a “clean deletion”). Alternatively, the 5′ editing template and the 3′ editing template can further comprise a region of complementarity to each other, and the region of complementarity can replace the IND in the DMPK gene. For example, in some embodiments, the 5′ PEgRNA is part of a dual prime editing composition that further comprises a 3′ PEgRNA, wherein the 5′ PEgRNA comprises a RTT comprising a sequence selected from sequence numbers 3459-3461 and 3467-3471, and the 3′ PEgRNA comprises a spacer comprising nucleotides 5-20 of sequence number 1525. In some embodiments, the 5′ PEgRNA is part of a dual prime editing composition that further comprises a 3′ PEgRNA, wherein the 5′ PEgRNA comprises a RTT comprising a sequence selected from sequence numbers 3462-3466, and the 3′ PEgRNA comprises a spacer comprising nucleotides 5-20 of sequence number 2353. In some embodiments, the 5′ PEgRNA is part of a dual prime editing composition that further comprises a 3′ PEgRNA, wherein the 5′ PEgRNA comprises a RTT comprising a sequence selected from sequence numbers 3472-3476, and the 3′ PEgRNA comprises a spacer comprising nucleotides 5-20 of sequence number 1936. In some embodiments, the 5′ PEgRNA is part of a dual prime editing composition that further comprises a 3′ PEgRNA, wherein the 5′ PEgRNA comprises a RTT comprising nucleotides x-40 of sequence number 3647, 3648, 3649, 3650, 3651, 3652, 3653, 3654, 3655, 3656, 3657, 3658, 3659, 3660, 3661, 3662, or 3663, wherein the 3′ PEgRNA comprises a spacer comprising nucleotides 5-20 of sequence number 1900, 1918, 1403, 1936, 2263, 2353, 1503, 2281, 2673, 1525, 2299, 2317, 1864, 1359, 2335, 1882, or 1381 respectively in the same order, and wherein x is any integer from 1 to 31. Preferably, the 3′ PEgRNA comprises a “clean deletion” RTT, for example, an RTT selected from Table 40 that has a Pairing 5′ spacer number that is 16. Each 5′ PEgRNA is assigned a RTT pairing number, which corresponds to a pairing 3′ spacer number in Table 39. The 5′ PEgRNA can be used in a dual prime editing system or composition with a 3′ PEgRNA comprising a spacer of the same pairing 3′ spacer number.

### Tables 17A and 17B

Tables 17A and 17B provide exemplary 5′ PEgRNAs and components that can be used with any prime editor containing a Cas9 protein capable of recognizing a NGG PAM sequence, wherein N is A, G, C, or T. The PEgRNAs exemplified in Tables 17A and 17B comprise: (a) a spacer comprising at its 3′ end a sequence corresponding to nucleotides 5-20 of sequence number 1001, (b) a gRNA core capable of binding to a Cas9 protein, and (c) an extension arm comprising (i) an editing template and (ii) a primer binding site (PBS) comprising at its 5′ end the sequence corresponding to sequence number 1016.

The PEgRNA spacer can be, for example, 16-22 nucleotides in length. In some embodiments, the PEgNA spacer is 16-20 nucleotides in length. In some embodiments, the PEgRNA spacer comprises nucleotides 5-20, 4-20, 3-20, 2-20, or 1-20 of sequence number 1001. In some embodiments, the PEgRNA spacer comprises the sequence of sequence number 1001. In some embodiments, the PEgRNA spacer is 20 nucleotides in length. Any of these spacers exemplified can be referred to as 5′ spacer No. 17.

The PBS can be, for example, 3 to 19 nucleotides in length. In some embodiments, the PBS is 3 to 17 nucleotides in length and corresponds to a PBS sequence in Table 17A. In some cases, a PBS length of no more than 3 nucleotides less than the PEgRNA spacer length is chosen.

The 5′ PEgRNAs of Tables 17A and 17B can be used in a dual prime editing system or composition further comprising any suitable 3′ PEgRNA. The prime editing system can be used, e.g., for editing a DMPK gene harboring 50 or more CTG repeats in the DMPK gene.

In some embodiments, a prime editing composition comprises a 5′ PEgRNA or component thereof exemplified in Tables 17A and 17B and a 3′ PEgRNA, wherein the editing template of the 5′ PEgRNA and the 3′ PEgRNA each comprises a region of complementarity to each other. For example, the editing template of the 5′ PEgRNA can comprise any of the RTT sequences provided in Table 37. The terms editing template and RTT may be used interchangeably. Each of the RTT sequences in Table 37 is assigned a RTT pairing number that reflects the complementarity relationship between a 5′ RTT and a 3′ RTT. The 5′ PEgRNA can be used in a dual prime editing system that further comprises a 3′ PEgRNA, e.g., a 3′ PEgRNA having a spacer sequence and a corresponding PBS as exemplified in Tables 20A-36B, wherein the 3′ PEgRNA comprises an editing template comprising a RTT sequence having a region of complementarity to the 5′ RTT. The 3′ RTT can comprise any of the RTT sequences provided in Table 38, wherein the 3′ RTT sequence has the same RTT Pairing number as that of the 5′ RTT. The editing template of the 5′ PEgRNA and the 3′ PEgRNA can be completely complementary to each other, wherein the 5′ editing template and the 3′ editing template have the same length and perfect complementarity to each other. Alternatively, the 5′ editing template can comprise a region of complementarity to the 3′ editing template, and further comprise a non-complementary region to the 3′ editing template. The 3′ editing template can comprise a region of complementarity to the 5′ editing template, and further comprise a non-complementary region to the 5′ editing template. The non-complementary region can be at the 5′ end or the 3′ end of the editing templates. Contacting the target DMPK gene with the prime editing system can result in deletion of the sequence between the 5′ PEgRNA nick and the 3′ PEgRNA nick (the IND) and insertion of the region of complementarity between the 5′ editing template and the 3′ editing template (the OD), or insertion of the region of complementarity between the 5′ editing template and the 3′ editing template as well as the non-complementarity regions (the RD), and hence replace the CTG expansion with the sequence of the OD or the RD. Each of the 5′ PEgRNA is assigned a RTT pairing number, and can be used in a dual prime editing composition with a 3′ PEgRNA comprising an RTT having the same RTT pairing number.

In some embodiments, a prime editing composition comprises a 5′ PEgRNA or component thereof exemplified in Tables 17A and 17B and a 3′ PEgRNA, wherein the editing template of the 5′ PEgRNA comprises a region of identity to the spacer of the 3′ PEgRNA. In some embodiments, the editing template of the 3′ PEgRNA comprises a region of identity to the spacer of the 5′ PEgRNA. In some embodiments, the 5′ editing template comprises a region of complementarity to a region on the second strand of the DMPK gene that is directly 3′ to nucleotide 3 of the search target sequence of the 3′ PEgRNA. In some embodiments, the 3′ editing template comprises a region of complementarity to a region on the first strand of the DMPK gene that is directly 3′ to nucleotide 3 of the search target sequence of the 5′ PEgRNA. For example, the extension arm of the 5′ PEgRNA can comprise an editing template comprising the sequence of any of the RTT sequences provided in Table 39. Each of the RTT sequences in Table 39 is assigned a pairing 3′ spacer number. The 5′ PEgRNA can be used in a dual prime editing system with a 3′ PEgRNA, wherein the 3′ PEgRNA comprises a spacer that has the Pairing 3′ spacer number assigned to the 5′ RTT in Table 39. Contacting the target DMPK gene with the prime editing system can result in deletion of the sequence between the 5′ PEgRNA nick and the 3′ PEgRNA nick (the IND) without insertion of any exogenous sequence (a “clean deletion”). Alternatively, the 5′ editing template and the 3′ editing template can further comprise a region of complementarity to each other, and the region of complementarity can replace the IND in the DMPK gene. For example, in some embodiments, the 5′ PEgRNA is part of a dual prime editing composition that further comprises a 3′ PEgRNA, wherein the 5′ PEgRNA comprises a RTT comprising a sequence selected from sequence numbers 3459-3461 and 3467-3471, and the 3′ PEgRNA comprises a spacer comprising nucleotides 5-20 of sequence number 1525. In some embodiments, the 5′ PEgRNA is part of a dual prime editing composition that further comprises a 3′ PEgRNA, wherein the 5′ PEgRNA comprises a RTT comprising a sequence selected from sequence numbers 3462-3466, and the 3′ PEgRNA comprises a spacer comprising nucleotides 5-20 of sequence number 2353. In some embodiments, the 5′ PEgRNA is part of a dual prime editing composition that further comprises a 3′ PEgRNA, wherein the 5′ PEgRNA comprises a RTT comprising a sequence selected from sequence numbers 3472-3476, and the 3′ PEgRNA comprises a spacer comprising nucleotides 5-20 of sequence number 1936. In some embodiments, the 5′ PEgRNA is part of a dual prime editing composition that further comprises a 3′ PEgRNA, wherein the 5′ PEgRNA comprises a RTT comprising nucleotides x-40 of sequence number 3647, 3648, 3649, 3650, 3651, 3652, 3653, 3654, 3655, 3656, 3657, 3658, 3659, 3660, 3661, 3662, or 3663, wherein the 3′ PEgRNA comprises a spacer comprising nucleotides 5-20 of sequence number 1900, 1918, 1403, 1936, 2263, 2353, 1503, 2281, 2673, 1525, 2299, 2317, 1864, 1359, 2335, 1882, or 1381 respectively in the same order, and wherein x is any integer from 1 to 31. Preferably, the 3′ PEgRNA comprises a “clean deletion” RTT, for example, an RTT selected from Table 40 that has a Pairing 5′ spacer number that is 17. Each 5′ PEgRNA is assigned a RTT pairing number, which corresponds to a pairing 3′ spacer number in Table 39. The 5′ PEgRNA can be used in a dual prime editing system or composition with a 3′ PEgRNA comprising a spacer of the same pairing 3′ spacer number.

### Tables 19A and 19B

Tables 19A and 19B provide exemplary 5′ PEgRNAs and components that can be used with any prime editor containing a Cas9 protein capable of recognizing a NGG PAM sequence, wherein N is A, G, C, or T. The PEgRNAs exemplified in Tables 19A and 19B comprise: (a) a spacer comprising at its 3′ end a sequence corresponding to nucleotides 5-20 of sequence number 1341, (b) a gRNA core capable of binding to a Cas9 protein, and (c) an extension arm comprising (i) an editing template and (ii) a primer binding site (PBS) comprising at its 5′ end the sequence corresponding to sequence number 1356.

The PEgRNA spacer can be, for example, 16-22 nucleotides in length. In some embodiments, the PEgNA spacer is 16-20 nucleotides in length. In some embodiments, the PEgRNA spacer comprises nucleotides 5-20, 4-20, 3-20, 2-20, or 1-20 of sequence number 1341. In some embodiments, the PEgRNA spacer comprises the sequence of sequence number 1341. In some embodiments, the PEgRNA spacer is 20 nucleotides in length. Any of these spacers exemplified can be referred to as 5′ spacer No. 19.

The PBS can be, for example, 3 to 19 nucleotides in length. In some embodiments, the PBS is 3 to 17 nucleotides in length and corresponds to a PBS sequence in Table 19A. In some cases, a PBS length of no more than 3 nucleotides less than the PEgRNA spacer length is chosen.

The 5′ PEgRNAs of Tables 19A and 19B can be used in a dual prime editing system or composition further comprising any suitable 3′ PEgRNA. The prime editing system can be used, e.g., for editing a DMPK gene harboring 50 or more CTG repeats in the DMPK gene.

In some embodiments, a prime editing composition comprises a 5′ PEgRNA or component thereof exemplified in Tables 19A and 19B and a 3′ PEgRNA, wherein the editing template of the 5′ PEgRNA and the 3′ PEgRNA each comprises a region of complementarity to each other. For example, the editing template of the 5′ PEgRNA can comprise any of the RTT sequences provided in Table 37. The terms editing template and RTT may be used interchangeably. Each of the RTT sequences in Table 37 is assigned a RTT pairing number that reflects the complementarity relationship between a 5′ RTT and a 3′ RTT. The 5′ PEgRNA can be used in a dual prime editing system that further comprises a 3′ PEgRNA, e.g., a 3′ PEgRNA having a spacer sequence and a corresponding PBS as exemplified in Tables 20A-36B, wherein the 3′ PEgRNA comprises an editing template comprising a RTT sequence having a region of complementarity to the 5′ RTT. The 3′ RTT can comprise any of the RTT sequences provided in Table 38, wherein the 3′ RTT sequence has the same RTT Pairing number as that of the 5′ RTT. The editing template of the 5′ PEgRNA and the 3′ PEgRNA can be completely complementary to each other, wherein the 5′ editing template and the 3′ editing template have the same length and perfect complementarity to each other. Alternatively, the 5′ editing template can comprise a region of complementarity to the 3′ editing template, and further comprise a non-complementary region to the 3′ editing template. The 3′ editing template can comprise a region of complementarity to the 5′ editing template, and further comprise a non-complementary region to the 5′ editing template. The non-complementary region can be at the 5′ end or the 3′ end of the editing templates. Contacting the target DMPK gene with the prime editing system can result in deletion of the sequence between the 5′ PEgRNA nick and the 3′ PEgRNA nick (the IND) and insertion of the region of complementarity between the 5′ editing template and the 3′ editing template (the OD), or insertion of the region of complementarity between the 5′ editing template and the 3′ editing template as well as the non-complementarity regions (the RD), and hence replace the CTG expansion with the sequence of the OD or the RD. Each of the 5′ PEgRNA is assigned a RTT pairing number, and can be used in a dual prime editing composition with a 3′ PEgRNA comprising an RTT having the same RTT pairing number.

In some embodiments, a prime editing composition comprises a 5′ PEgRNA or component thereof exemplified in Tables 19A and 19B and a 3′ PEgRNA, wherein the editing template of the 5′ PEgRNA comprises a region of identity to the spacer of the 3′ PEgRNA. In some embodiments, the editing template of the 3′ PEgRNA comprises a region of identity to the spacer of the 5′ PEgRNA. In some embodiments, the 5′ editing template comprises a region of complementarity to a region on the second strand of the DMPK gene that is directly 3′ to nucleotide 3 of the search target sequence of the 3′ PEgRNA. In some embodiments, the 3′ editing template comprises a region of complementarity to a region on the first strand of the DMPK gene that is directly 3′ to nucleotide 3 of the search target sequence of the 5′ PEgRNA. For example, the extension arm of the 5′ PEgRNA can comprise an editing template comprising the sequence of any of the RTT sequences provided in Table 39. Each of the RTT sequences in Table 39 is assigned a pairing 3′ spacer number. The 5′ PEgRNA can be used in a dual prime editing system with a 3′ PEgRNA, wherein the 3′ PEgRNA comprises a spacer that has the Pairing 3′ spacer number assigned to the 5′ RTT in Table 39. Contacting the target DMPK gene with the prime editing system can result in deletion of the sequence between the 5′ PEgRNA nick and the 3′ PEgRNA nick (the IND) without insertion of any exogenous sequence (a “clean deletion”). Alternatively, the 5′ editing template and the 3′ editing template can further comprise a region of complementarity to each other, and the region of complementarity can replace the IND in the DMPK gene. For example, in some embodiments, the 5′ PEgRNA is part of a dual prime editing composition that further comprises a 3′ PEgRNA, wherein the 5′ PEgRNA comprises a RTT comprising a sequence selected from sequence numbers 3459-3461 and 3467-3471, and the 3′ PEgRNA comprises a spacer comprising nucleotides 5-20 of sequence number 1525. In some embodiments, the 5′ PEgRNA is part of a dual prime editing composition that further comprises a 3′ PEgRNA, wherein the 5′ PEgRNA comprises a RTT comprising a sequence selected from sequence numbers 3462-3466, and the 3′ PEgRNA comprises a spacer comprising nucleotides 5-20 of sequence number 2353. In some embodiments, the 5′ PEgRNA is part of a dual prime editing composition that further comprises a 3′ PEgRNA, wherein the 5′ PEgRNA comprises a RTT comprising a sequence selected from sequence numbers 3472-3476, and the 3′ PEgRNA comprises a spacer comprising nucleotides 5-20 of sequence number 1936. In some embodiments, the 5′ PEgRNA is part of a dual prime editing composition that further comprises a 3′ PEgRNA, wherein the 5′ PEgRNA comprises a RTT comprising nucleotides x-40 of sequence number 3647, 3648, 3649, 3650, 3651, 3652, 3653, 3654, 3655, 3656, 3657, 3658, 3659, 3660, 3661, 3662, or 3663, wherein the 3′ PEgRNA comprises a spacer comprising nucleotides 5-20 of sequence number 1900, 1918, 1403, 1936, 2263, 2353, 1503, 2281, 2673, 1525, 2299, 2317, 1864, 1359, 2335, 1882, or 1381 respectively in the same order, and wherein x is any integer from 1 to 31. Preferably, the 3′ PEgRNA comprises a “clean deletion” RTT, for example, an RTT selected from Table 40 that has a Pairing 5′ spacer number that is 19. Each 5′ PEgRNA is assigned a RTT pairing number, which corresponds to a pairing 3′ spacer number in Table 39. The 5′ PEgRNA can be used in a dual prime editing system or composition with a 3′ PEgRNA comprising a spacer of the same pairing 3′ spacer number.

### Tables 20A and 20B

Tables 20A and 20B provide exemplary 3′ PEgRNAs and components that can be used with any prime editor containing a Cas9 protein capable of recognizing a NGG PAM sequence, wherein N is A, G, C, or T. The PEgRNAs exemplified in Tables 20A and 20B comprise: (a) a spacer comprising at its 3′ end a sequence corresponding to nucleotides 5-20 of sequence number 1359, (b) a gRNA core capable of binding to a Cas9 protein, and (c) an extension arm comprising (i) an editing template and (ii) a primer binding site (PBS) comprising at its 5′ end the sequence corresponding to sequence number 1374.

The PEgRNA spacer can be, for example, 16-22 nucleotides in length. In some embodiments, the PEgNA spacer is 16-20 nucleotides in length. In some embodiments, the PEgRNA spacer comprises nucleotides 5-20, 4-20, 3-20, 2-20, or 1-20 of sequence number 1359. In some embodiments, the PEgRNA spacer comprises the sequence of sequence number 1359. In some embodiments, the PEgRNA spacer is 20 nucleotides in length. Any of these spacers exemplified can be referred to as 3′ spacer No. 1.

The PBS can be, for example, 3 to 19 nucleotides in length. In some embodiments, the PBS is 3 to 17 nucleotides in length and corresponds to a PBS sequence in Table 20A. In some cases, a PBS length of no more than 3 nucleotides less than the PEgRNA spacer length is chosen.

The 3′ PEgRNAs of Tables 20A and 20B can be used in a dual prime editing system or composition further comprising any suitable 5′ PEgRNA. The prime editing system can be used, e.g., for editing a DMPK gene harboring 50 or more CTG repeats in the DMPK gene.

In some embodiments, a prime editing composition comprises a 3′ PEgRNA or component thereof exemplified in Tables 20A and 20B and a 5′ PEgRNA, wherein the editing template of the 3′ PEgRNA and the 5′ PEgRNA each comprises a region of complementarity to each other. For example, the editing template of the 3′ PEgRNA can comprise any of the RTT sequences provided in Table 38. The terms editing template and RTT may be used interchangeably. Each of the RTT sequences in Table 38 is assigned a RTT pairing number that reflects the complementarity relationship between a 5′ RTT and a 3′ RTT. The 3′ PEgRNA can be used in a dual prime editing system that further comprises a 5′ PEgRNA, e.g., a 5′ PEgRNA having a spacer sequence and a corresponding PBS as exemplified in Tables 1A-19B, wherein the 5′ PEgRNA comprises an editing template comprising a RTT sequence having a region of complementarity to the 3′ RTT. The 5′ RTT can comprise any of the RTT sequences provided in Table 37, wherein the 5′ RTT sequence has the same RTT Pairing number as that of the 3′ RTT. The editing template of the 5′ PEgRNA and the 3′ PEgRNA can be completely complementary to each other, wherein the 5′ editing template and the 3′ editing template have the same length and perfect complementarity to each other. Alternatively, the 5′ editing template can comprise a region of complementarity to the 3′ editing template, and further comprise a non-complementary region to the 3′ editing template. The 3′ editing template can comprise a region of complementarity to the 5′ editing template, and further comprise a non-complementary region to the 5′ editing template. The non-complementary region can be at the 5′ end or the 3′ end of the editing templates. Contacting the target DMPK gene with the prime editing system can result in deletion of the sequence between the 5′ PEgRNA nick and the 3′ PEgRNA nick (the IND) and insertion of the region of complementarity between the 5′ editing template and the 3′ editing template (the OD), or insertion of the region of complementarity between the 5′ editing template and the 3′ editing template as well as the non-complementarity regions (the RD), and hence replace the CTG expansion with the sequence of the OD or the RD. Each of the 3′ PEgRNA is assigned a RTT pairing number, and can be used in a dual prime editing composition with a 5′ PEgRNA comprising an RTT having the same RTT pairing number.

In some embodiments, a prime editing composition comprises a 3′ PEgRNA or component thereof exemplified in Tables 20A and 20B and a 5′ PEgRNA, wherein the editing template of the 3′ PEgRNA comprises a region of identity to the spacer of the 5′ PEgRNA. In some embodiments, the editing template of the 5′ PEgRNA comprises a region of identity to the spacer of the 3′ PEgRNA. In some embodiments, the 3′ editing template comprises a region of complementarity to a region on the first strand of the DMPK gene that is directly 3′ to nucleotide 3 of the search target sequence of the 5′ PEgRNA. In some embodiments, the 5′ editing template comprises a region of complementarity to a region on the second strand of the DMPK gene that is directly 3′ to nucleotide 3 of the search target sequence of the 3′ PEgRNA. For example, the extension arm of the 3′ PEgRNA can comprise an editing template comprising the sequence of any of the RTT sequences provided in Table 40. Each of the RTT sequences in Table 40 is assigned a pairing 5′ spacer number. The 3′ PEgRNA can be used in a dual prime editing system with a 5′ PEgRNA, wherein the 5′ PEgRNA comprises a spacer that has the Pairing 5′ spacer number assigned to the 3′ RTT in Table 40. Contacting the target DMPK gene with the prime editing system can result in deletion of the sequence between the 5′ PEgRNA nick and the 3′ PEgRNA nick (the IND) without insertion of any exogenous sequence (a “clean deletion”). Alternatively, the 5′ editing template and the 3′ editing template can further comprise a region of complementarity to each other, and the region of complementarity can replace the IND in the DMPK gene. For example, in some embodiments, the 3′ PEgRNA is part of a dual prime editing composition that further comprises a 5′ PEgRNA, wherein the 3′ PEgRNA comprises a RTT comprising a sequence selected from sequence numbers 3477-3479, and the 5′ PEgRNA comprises a spacer comprising nucleotides 5-20 of sequence number 484. In some embodiments, the 3′ PEgRNA is part of a dual prime editing composition that further comprises a 5′ PEgRNA, wherein the 3′ PEgRNA comprises a RTT comprising a sequence selected from sequence numbers 3480-3484, and the 5′ PEgRNA comprises a spacer comprising nucleotides 5-20 of sequence number 77. In some embodiments, the 3′ PEgRNA is part of a dual prime editing composition that further comprises a 5′ PEgRNA, wherein the 3′ PEgRNA comprises a RTT comprising a sequence selected from sequence numbers 3485-3489, and the 5′ PEgRNA comprises a spacer comprising nucleotides 5-20 of sequence number 590. In some embodiments, the 3′ PEgRNA is part of a dual prime editing composition that further comprises a 5′ PEgRNA, wherein the 3′ PEgRNA comprises a RTT comprising a sequence selected from sequence numbers 3490-3494, and the 5′ PEgRNA comprises a spacer comprising nucleotides 5-20 of sequence number 1019. In some embodiments, the 3′ PEgRNA is part of a dual prime editing composition that further comprises a 5′ PEgRNA, wherein the 3′ PEgRNA comprises a RTT comprising nucleotides x-40 of sequence number 3664, 3665, 3666, 3667, 3668, 3669, 3670, 3671, 3672, 3673, 3674, 3675, 3676, 3677, 3678, 3679, 3680, 3681, or 3682, wherein the 5′ PEgRNA comprises a spacer comprising nucleotides 5-20 of sequence number 518, 536, 554, 572, 590, 39, 912, 929, 947, 965, 983, 1001, 1019, 1341, 20, 77, 484, 58, or 1 respectively in the same order, and wherein x is any integer from 1 to 31. Each 3′ PEgRNA is assigned a RTT pairing number, which corresponds to a pairing 5′ spacer number in Table 40. The 3′ PEgRNA can be used in a dual prime editing system or composition with a 5′ PEgRNA comprising a spacer of the same pairing 5′ spacer number. Preferably, the 5′ PEgRNA comprises a “clean deletion” RTT, for example, an RTT selected from Table 39 that has a Pairing 3′ spacer number that is 1.

### Tables 21A and 21B

Tables 21A and 21B provide exemplary 3′ PEgRNAs and components that can be used with any prime editor containing a Cas9 protein capable of recognizing a NGG PAM sequence, wherein N is A, G, C, or T. The PEgRNAs exemplified in Tables 21A and 21B comprise: (a) a spacer comprising at its 3′ end a sequence corresponding to nucleotides 5-20 of sequence number 1381, (b) a gRNA core capable of binding to a Cas9 protein, and (c) an extension arm comprising (i) an editing template and (ii) a primer binding site (PBS) comprising at its 5′ end the sequence corresponding to sequence number 1396.

The PEgRNA spacer can be, for example, 16-22 nucleotides in length. In some embodiments, the PEgNA spacer is 16-20 nucleotides in length. In some embodiments, the PEgRNA spacer comprises nucleotides 5-20, 4-20, 3-20, 2-20, or 1-20 of sequence number 1381. In some embodiments, the PEgRNA spacer comprises the sequence of sequence number 1381. In some embodiments, the PEgRNA spacer is 20 nucleotides in length. Any of these spacers exemplified can be referred to as 3′ spacer No. 2.

The PBS can be, for example, 3 to 19 nucleotides in length. In some embodiments, the PBS is 3 to 17 nucleotides in length and corresponds to a PBS sequence in Table 20A. In some cases, a PBS length of no more than 3 nucleotides less than the PEgRNA spacer length is chosen.

The 3′ PEgRNAs of Tables 21A and 21B can be used in a dual prime editing system or composition further comprising any suitable 5′ PEgRNA. The prime editing system can be used, e.g., for editing a DMPK gene harboring 50 or more CTG repeats in the DMPK gene.

In some embodiments, a prime editing composition comprises a 3′ PEgRNA or component thereof exemplified in Tables 21A and 21B and a 5′ PEgRNA, wherein the editing template of the 3′ PEgRNA and the 5′ PEgRNA each comprises a region of complementarity to each other. For example, the editing template of the 3′ PEgRNA can comprise any of the RTT sequences provided in Table 38. The terms editing template and RTT may be used interchangeably. Each of the RTT sequences in Table 38 is assigned a RTT pairing number that reflects the complementarity relationship between a 5′ RTT and a 3′ RTT. The 3′ PEgRNA can be used in a dual prime editing system that further comprises a 5′ PEgRNA, e.g., a 5′ PEgRNA having a spacer sequence and a corresponding PBS as exemplified in Tables 1A-19B, wherein the 5′ PEgRNA comprises an editing template comprising a RTT sequence having a region of complementarity to the 3′ RTT. The 5′ RTT can comprise any of the RTT sequences provided in Table 37, wherein the 5′ RTT sequence has the same RTT Pairing number as that of the 3′ RTT. The editing template of the 5′ PEgRNA and the 3′ PEgRNA can be completely complementary to each other, wherein the 5′ editing template and the 3′ editing template have the same length and perfect complementarity to each other. Alternatively, the 5′ editing template can comprise a region of complementarity to the 3′ editing template, and further comprise a non-complementary region to the 3′ editing template. The 3′ editing template can comprise a region of complementarity to the 5′ editing template, and further comprise a non-complementary region to the 5′ editing template. The non-complementary region can be at the 5′ end or the 3′ end of the editing templates. Contacting the target DMPK gene with the prime editing system can result in deletion of the sequence between the 5′ PEgRNA nick and the 3′ PEgRNA nick (the IND) and insertion of the region of complementarity between the 5′ editing template and the 3′ editing template (the OD), or insertion of the region of complementarity between the 5′ editing template and the 3′ editing template as well as the non-complementarity regions (the RD), and hence replace the CTG expansion with the sequence of the OD or the RD. Each of the 3′ PEgRNA is assigned a RTT pairing number, and can be used in a dual prime editing composition with a 5′ PEgRNA comprising an RTT having the same RTT pairing number.

In some embodiments, a prime editing composition comprises a 3′ PEgRNA or component thereof exemplified in Tables 21A and 21B and a 5′ PEgRNA, wherein the editing template of the 3′ PEgRNA comprises a region of identity to the spacer of the 5′ PEgRNA. In some embodiments, the editing template of the 5′ PEgRNA comprises a region of identity to the spacer of the 3′ PEgRNA. In some embodiments, the 3′ editing template comprises a region of complementarity to a region on the first strand of the DMPK gene that is directly 3′ to nucleotide 3 of the search target sequence of the 5′ PEgRNA. In some embodiments, the 5′ editing template comprises a region of complementarity to a region on the second strand of the DMPK gene that is directly 3′ to nucleotide 3 (of the search target sequence of the 3′ PEgRNA. For example, the extension arm of the 3′ PEgRNA can comprise an editing template comprising the sequence of any of the RTT sequences provided in Table 40. Each of the RTT sequences in Table 40 is assigned a pairing 5′ spacer number. The 3′ PEgRNA can be used in a dual prime editing system with a 5′ PEgRNA, wherein the 5′ PEgRNA comprises a spacer that has the Pairing 5′ spacer number assigned to the 3′ RTT in Table 40. Contacting the target DMPK gene with the prime editing system can result in deletion of the sequence between the 5′ PEgRNA nick and the 3′ PEgRNA nick (the IND) without insertion of any exogenous sequence (a “clean deletion”). Alternatively, the 5′ editing template and the 3′ editing template can further comprise a region of complementarity to each other, and the region of complementarity can replace the IND in the DMPK gene. For example, in some embodiments, the 3′ PEgRNA is part of a dual prime editing composition that further comprises a 5′ PEgRNA, wherein the 3′ PEgRNA comprises a RTT comprising a sequence selected from sequence numbers 3477-3479, and the 5′ PEgRNA comprises a spacer comprising nucleotides 5-20 of sequence number 484. In some embodiments, the 3′ PEgRNA is part of a dual prime editing composition that further comprises a 5′ PEgRNA, wherein the 3′ PEgRNA comprises a RTT comprising a sequence selected from sequence numbers 3480-3484, and the 5′ PEgRNA comprises a spacer comprising nucleotides 5-20 of sequence number 77. In some embodiments, the 3′ PEgRNA is part of a dual prime editing composition that further comprises a 5′ PEgRNA, wherein the 3′ PEgRNA comprises a RTT comprising a sequence selected from sequence numbers 3485-3489, and the 5′ PEgRNA comprises a spacer comprising nucleotides 5-20 of sequence number 590. In some embodiments, the 3′ PEgRNA is part of a dual prime editing composition that further comprises a 5′ PEgRNA, wherein the 3′ PEgRNA comprises a RTT comprising a sequence selected from sequence numbers 3490-3494, and the 5′ PEgRNA comprises a spacer comprising nucleotides 5-20 of sequence number 1019. In some embodiments, the 3′ PEgRNA is part of a dual prime editing composition that further comprises a 5′ PEgRNA, wherein the 3′ PEgRNA comprises a RTT comprising nucleotides x-40 of sequence number 3664, 3665, 3666, 3667, 3668, 3669, 3670, 3671, 3672, 3673, 3674, 3675, 3676, 3677, 3678, 3679, 3680, 3681, or 3682, wherein the 5′ PEgRNA comprises a spacer comprising nucleotides 5-20 of sequence number 518, 536, 554, 572, 590, 39, 912, 929, 947, 965, 983, 1001, 1019, 1341, 20, 77, 484, 58, or 1 respectively in the same order, and wherein x is any integer from 1 to 31. Each 3′ PEgRNA is assigned a RTT pairing number, which corresponds to a pairing 5′ spacer number in Table 40. The 3′ PEgRNA can be used in a dual prime editing system or composition with a 5′ PEgRNA comprising a spacer of the same pairing 5′ spacer number. Preferably, the 5′ PEgRNA comprises a “clean deletion” RTT, for example, an RTT selected from Table 39 that has a Pairing 3′ spacer number that is 2.

### Tables 22A and 22B

Tables 22A and 22B provide exemplary 3′ PEgRNAs and components that can be used with any prime editor containing a Cas9 protein capable of recognizing a NGG PAM sequence, wherein N is A, G, C, or T. The PEgRNAs exemplified in Tables 22A and 22B comprise: (a) a spacer comprising at its 3′ end a sequence corresponding to nucleotides 5-20 of sequence number 1403, (b) a gRNA core capable of binding to a Cas9 protein, and (c) an extension arm comprising (i) an editing template and (ii) a primer binding site (PBS) comprising at its 5′ end the sequence corresponding to sequence number 1418.

The PEgRNA spacer can be, for example, 16-22 nucleotides in length. In some embodiments, the PEgNA spacer is 16-20 nucleotides in length. In some embodiments, the PEgRNA spacer comprises nucleotides 5-20, 4-20, 3-20, 2-20, or 1-20 of sequence number 1403. In some embodiments, the PEgRNA spacer comprises the sequence of sequence number 1403. In some embodiments, the PEgRNA spacer is 20 nucleotides in length. Any of these spacers exemplified can be referred to as 3′ spacer No. 3.

The PBS can be, for example, 3 to 19 nucleotides in length. In some embodiments, the PBS is 3 to 17 nucleotides in length and corresponds to a PBS sequence in Table 20A. In some cases, a PBS length of no more than 3 nucleotides less than the PEgRNA spacer length is chosen.

The 3′ PEgRNAs of Tables 22A and 22B can be used in a dual prime editing system or composition further comprising any suitable 5′ PEgRNA. The prime editing system can be used, e.g., for editing a DMPK gene harboring 50 or more CTG repeats in the DMPK gene.

In some embodiments, a prime editing composition comprises a 3′ PEgRNA or component thereof exemplified in Tables 22A and 22B and a 5′ PEgRNA, wherein the editing template of the 3′ PEgRNA and the 5′ PEgRNA each comprises a region of complementarity to each other. For example, the editing template of the 3′ PEgRNA can comprise any of the RTT sequences provided in Table 38. The terms editing template and RTT may be used interchangeably. Each of the RTT sequences in Table 38 is assigned a RTT pairing number that reflects the complementarity relationship between a 5′ RTT and a 3′ RTT. The 3′ PEgRNA can be used in a dual prime editing system that further comprises a 5′ PEgRNA, e.g., a 5′ PEgRNA having a spacer sequence and a corresponding PBS as exemplified in Tables 1A-19B, wherein the 5′ PEgRNA comprises an editing template comprising a RTT sequence having a region of complementarity to the 3′ RTT. The 5′ RTT can comprise any of the RTT sequences provided in Table 37, wherein the 5′ RTT sequence has the same RTT Pairing number as that of the 3′ RTT. The editing template of the 5′ PEgRNA and the 3′ PEgRNA can be completely complementary to each other, wherein the 5′ editing template and the 3′ editing template have the same length and perfect complementarity to each other. Alternatively, the 5′ editing template can comprise a region of complementarity to the 3′ editing template, and further comprise a non-complementary region to the 3′ editing template. The 3′ editing template can comprise a region of complementarity to the 5′ editing template, and further comprise a non-complementary region to the 5′ editing template. The non-complementary region can be at the 5′ end or the 3′ end of the editing templates. Contacting the target DMPK gene with the prime editing system can result in deletion of the sequence between the 5′ PEgRNA nick and the 3′ PEgRNA nick (the IND) and insertion of the region of complementarity between the 5′ editing template and the 3′ editing template (the OD), or insertion of the region of complementarity between the 5′ editing template and the 3′ editing template as well as the non-complementarity regions (the RD), and hence replace the CTG expansion with the sequence of the OD or the RD. Each of the 3′ PEgRNA is assigned a RTT pairing number, and can be used in a dual prime editing composition with a 5′ PEgRNA comprising an RTT having the same RTT pairing number.

In some embodiments, a prime editing composition comprises a 3′ PEgRNA or component thereof exemplified in Tables 22A and 22B and a 5′ PEgRNA, wherein the editing template of the 3′ PEgRNA comprises a region of identity to the spacer of the 5′ PEgRNA. In some embodiments, the editing template of the 5′ PEgRNA comprises a region of identity to the spacer of the 3′ PEgRNA. In some embodiments, the 3′ editing template comprises a region of complementarity to a region on the first strand of the DMPK gene that is directly 3′ to nucleotide 3 of the search target sequence of the 5′ PEgRNA. In some embodiments, the 5′ editing template comprises a region of complementarity to a region on the second strand of the DMPK gene that is directly 3′ to nucleotide 3 of the search target sequence of the 3′ PEgRNA. For example, the extension arm of the 3′ PEgRNA can comprise an editing template comprising the sequence of any of the RTT sequences provided in Table 40. Each of the RTT sequences in Table 40 is assigned a pairing 5′ spacer number. The 3′ PEgRNA can be used in a dual prime editing system with a 5′ PEgRNA, wherein the 5′ PEgRNA comprises a spacer that has the Pairing 5′ spacer number assigned to the 3′ RTT in Table 40. Contacting the target DMPK gene with the prime editing system can result in deletion of the sequence between the 5′ PEgRNA nick and the 3′ PEgRNA nick (the IND) without insertion of any exogenous sequence (a “clean deletion”). Alternatively, the 5′ editing template and the 3′ editing template can further comprise a region of complementarity to each other, and the region of complementarity can replace the IND in the DMPK gene. For example, in some embodiments, the 3′ PEgRNA is part of a dual prime editing composition that further comprises a 5′ PEgRNA, wherein the 3′ PEgRNA comprises a RTT comprising a sequence selected from sequence numbers 3477-3479, and the 5′ PEgRNA comprises a spacer comprising nucleotides 5-20 of sequence number 484. In some embodiments, the 3′ PEgRNA is part of a dual prime editing composition that further comprises a 5′ PEgRNA, wherein the 3′ PEgRNA comprises a RTT comprising a sequence selected from sequence numbers 3480-3484, and the 5′ PEgRNA comprises a spacer comprising nucleotides 5-20 of sequence number 77. In some embodiments, the 3′ PEgRNA is part of a dual prime editing composition that further comprises a 5′ PEgRNA, wherein the 3′ PEgRNA comprises a RTT comprising a sequence selected from sequence numbers 3485-3489, and the 5′ PEgRNA comprises a spacer comprising nucleotides 5-20 of sequence number 590. In some embodiments, the 3′ PEgRNA is part of a dual prime editing composition that further comprises a 5′ PEgRNA, wherein the 3′ PEgRNA comprises a RTT comprising a sequence selected from sequence numbers 3490-3494, and the 5′ PEgRNA comprises a spacer comprising nucleotides 5-20 of sequence number 1019. In some embodiments, the 3′ PEgRNA is part of a dual prime editing composition that further comprises a 5′ PEgRNA, wherein the 3′ PEgRNA comprises a RTT comprising nucleotides x-40 of sequence number 3664, 3665, 3666, 3667, 3668, 3669, 3670, 3671, 3672, 3673, 3674, 3675, 3676, 3677, 3678, 3679, 3680, 3681, or 3682, wherein the 5′ PEgRNA comprises a spacer comprising nucleotides 5-20 of sequence number 518, 536, 554, 572, 590, 39, 912, 929, 947, 965, 983, 1001, 1019, 1341, 20, 77, 484, 58, or 1 respectively in the same order, and wherein x is any integer from 1 to 31. Each 3′ PEgRNA is assigned a RTT pairing number, which corresponds to a pairing 5′ spacer number in Table 40. The 3′ PEgRNA can be used in a dual prime editing system or composition with a 5′ PEgRNA comprising a spacer of the same pairing 5′ spacer number. Preferably, the 5′ PEgRNA comprises a “clean deletion” RTT, for example, an RTT selected from Table 39 that has a Pairing 3′ spacer number that is 3.

### Tables 23A and 23B

Tables 23A and 23B provide exemplary 3′ PEgRNAs and components that can be used with any prime editor containing a Cas9 protein capable of recognizing a NGG PAM sequence, wherein N is A, G, C, or T. The PEgRNAs exemplified in Tables 23A and 23B comprise: (a) a spacer comprising at its 3′ end a sequence corresponding to nucleotides 5-20 of sequence number 1503, (b) a gRNA core capable of binding to a Cas9 protein, and (c) an extension arm comprising (i) an editing template and (ii) a primer binding site (PBS) comprising at its 5′ end the sequence corresponding to sequence number 1518.

The PEgRNA spacer can be, for example, 16-22 nucleotides in length. In some embodiments, the PEgNA spacer is 16-20 nucleotides in length. In some embodiments, the PEgRNA spacer comprises nucleotides 5-20, 4-20, 3-20, 2-20, or 1-20 of sequence number 1503. In some embodiments, the PEgRNA spacer comprises the sequence of sequence number 1503. In some embodiments, the PEgRNA spacer is 20 nucleotides in length. Any of these spacers exemplified can be referred to as 3′ spacer No. 4.

The PBS can be, for example, 3 to 19 nucleotides in length. In some embodiments, the PBS is 3 to 17 nucleotides in length and corresponds to a PBS sequence in Table 20A. In some cases, a PBS length of no more than 3 nucleotides less than the PEgRNA spacer length is chosen.

The 3′ PEgRNAs of Tables 23A and 23B can be used in a dual prime editing system or composition further comprising any suitable 5′ PEgRNA. The prime editing system can be used, e.g., for editing a DMPK gene harboring 50 or more CTG repeats in the DMPK gene.

In some embodiments, a prime editing composition comprises a 3′ PEgRNA or component thereof exemplified in Tables 23A and 23B and a 5′ PEgRNA, wherein the editing template of the 3′ PEgRNA and the 5′ PEgRNA each comprises a region of complementarity to each other. For example, the editing template of the 3′ PEgRNA can comprise any of the RTT sequences provided in Table 38. The terms editing template and RTT may be used interchangeably. Each of the RTT sequences in Table 38 is assigned a RTT pairing number that reflects the complementarity relationship between a 5′ RTT and a 3′ RTT. The 3′ PEgRNA can be used in a dual prime editing system that further comprises a 5′ PEgRNA, e.g., a 5′ PEgRNA having a spacer sequence and a corresponding PBS as exemplified in Tables 1A-19B, wherein the 5′ PEgRNA comprises an editing template comprising a RTT sequence having a region of complementarity to the 3′ RTT. The 5′ RTT can comprise any of the RTT sequences provided in Table 37, wherein the 5′ RTT sequence has the same RTT Pairing number as that of the 3′ RTT. The editing template of the 5′ PEgRNA and the 3′ PEgRNA can be completely complementary to each other, wherein the 5′ editing template and the 3′ editing template have the same length and perfect complementarity to each other. Alternatively, the 5′ editing template can comprise a region of complementarity to the 3′ editing template, and further comprise a non-complementary region to the 3′ editing template. The 3′ editing template can comprise a region of complementarity to the 5′ editing template, and further comprise a non-complementary region to the 5′ editing template. The non-complementary region can be at the 5′ end or the 3′ end of the editing templates. Contacting the target DMPK gene with the prime editing system can result in deletion of the sequence between the 5′ PEgRNA nick and the 3′ PEgRNA nick (the IND) and insertion of the region of complementarity between the 5′ editing template and the 3′ editing template (the OD), or insertion of the region of complementarity between the 5′ editing template and the 3′ editing template as well as the non-complementarity regions (the RD), and hence replace the CTG expansion with the sequence of the OD or the RD. Each of the 3′ PEgRNA is assigned a RTT pairing number, and can be used in a dual prime editing composition with a 5′ PEgRNA comprising an RTT having the same RTT pairing number.

In some embodiments, a prime editing composition comprises a 3′ PEgRNA or component thereof exemplified in Tables 23A and 23B and a 5′ PEgRNA, wherein the editing template of the 3′ PEgRNA comprises a region of identity to the spacer of the 5′ PEgRNA. In some embodiments, the editing template of the 5′ PEgRNA comprises a region of identity to the spacer of the 3′ PEgRNA. In some embodiments, the 3′ editing template comprises a region of complementarity to a region on the first strand of the DMPK gene that is directly 3′ to nucleotide 3 of the search target sequence of the 5′ PEgRNA. In some embodiments, the 5′ editing template comprises a region of complementarity to a region on the second strand of the DMPK gene that is directly 3′ to nucleotide 3 of the search target sequence of the 3′ PEgRNA. For example, the extension arm of the 3′ PEgRNA can comprise an editing template comprising the sequence of any of the RTT sequences provided in Table 40. Each of the RTT sequences in Table 40 is assigned a pairing 5′ spacer number. The 3′ PEgRNA can be used in a dual prime editing system with a 5′ PEgRNA, wherein the 5′ PEgRNA comprises a spacer that has the Pairing 5′ spacer number assigned to the 3′ RTT in Table 40. Contacting the target DMPK gene with the prime editing system can result in deletion of the sequence between the 5′ PEgRNA nick and the 3′ PEgRNA nick (the IND) without insertion of any exogenous sequence (a “clean deletion”). Alternatively, the 5′ editing template and the 3′ editing template can further comprise a region of complementarity to each other, and the region of complementarity can replace the IND in the DMPK gene. For example, in some embodiments, the 3′ PEgRNA is part of a dual prime editing composition that further comprises a 5′ PEgRNA, wherein the 3′ PEgRNA comprises a RTT comprising a sequence selected from sequence numbers 3477-3479, and the 5′ PEgRNA comprises a spacer comprising nucleotides 5-20 of sequence number 484. In some embodiments, the 3′ PEgRNA is part of a dual prime editing composition that further comprises a 5′ PEgRNA, wherein the 3′ PEgRNA comprises a RTT comprising a sequence selected from sequence numbers 3480-3484, and the 5′ PEgRNA comprises a spacer comprising nucleotides 5-20 of sequence number 77. In some embodiments, the 3′ PEgRNA is part of a dual prime editing composition that further comprises a 5′ PEgRNA, wherein the 3′ PEgRNA comprises a RTT comprising a sequence selected from sequence numbers 3485-3489, and the 5′ PEgRNA comprises a spacer comprising nucleotides 5-20 of sequence number 590. In some embodiments, the 3′ PEgRNA is part of a dual prime editing composition that further comprises a 5′ PEgRNA, wherein the 3′ PEgRNA comprises a RTT comprising a sequence selected from sequence numbers 3490-3494, and the 5′ PEgRNA comprises a spacer comprising nucleotides 5-20 of sequence number 1019. In some embodiments, the 3′ PEgRNA is part of a dual prime editing composition that further comprises a 5′ PEgRNA, wherein the 3′ PEgRNA comprises a RTT comprising nucleotides x-40 of sequence number 3664, 3665, 3666, 3667, 3668, 3669, 3670, 3671, 3672, 3673, 3674, 3675, 3676, 3677, 3678, 3679, 3680, 3681, or 3682, wherein the 5′ PEgRNA comprises a spacer comprising nucleotides 5-20 of sequence number 518, 536, 554, 572, 590, 39, 912, 929, 947, 965, 983, 1001, 1019, 1341, 20, 77, 484, 58, or 1 respectively in the same order, and wherein x is any integer from 1 to 31. Each 3′ PEgRNA is assigned a RTT pairing number, which corresponds to a pairing 5′ spacer number in Table 40. The 3′ PEgRNA can be used in a dual prime editing system or composition with a 5′ PEgRNA comprising a spacer of the same pairing 5′ spacer number. Preferably, the 5′ PEgRNA comprises a “clean deletion” RTT, for example, an RTT selected from Table 39 that has a Pairing 3′ spacer number that is 4.

### Tables 24A and 24B

Tables 24A and 24B provide exemplary 3′ PEgRNAs and components that can be used with any prime editor containing a Cas9 protein capable of recognizing a NGG PAM sequence, wherein N is A, G, C, or T. The PEgRNAs exemplified in Tables 24A and 24B comprise: (a) a spacer comprising at its 3′ end a sequence corresponding to nucleotides 5-20 of sequence number 1525, (b) a gRNA core capable of binding to a Cas9 protein, and (c) an extension arm comprising (i) an editing template and (ii) a primer binding site (PBS) comprising at its 5′ end the sequence corresponding to sequence number 1540.

The PEgRNA spacer can be, for example, 16-22 nucleotides in length. In some embodiments, the PEgNA spacer is 16-20 nucleotides in length. In some embodiments, the PEgRNA spacer comprises nucleotides 5-20, 4-20, 3-20, 2-20, or 1-20 of sequence number 1525. In some embodiments, the PEgRNA spacer comprises the sequence of sequence number 1525. In some embodiments, the PEgRNA spacer is 20 nucleotides in length. Any of these spacers exemplified can be referred to as 3′ spacer No. 5.

The PBS can be, for example, 3 to 19 nucleotides in length. In some embodiments, the PBS is 3 to 17 nucleotides in length and corresponds to a PBS sequence in Table 20A. In some cases, a PBS length of no more than 3 nucleotides less than the PEgRNA spacer length is chosen.

The 3′ PEgRNAs of Tables 24A and 24B can be used in a dual prime editing system or composition further comprising any suitable 5′ PEgRNA. The prime editing system can be used, e.g., for editing a DMPK gene harboring 50 or more CTG repeats in the DMPK gene.

In some embodiments, a prime editing composition comprises a 3′ PEgRNA or component thereof exemplified in Tables 24A and 24B and a 5′ PEgRNA, wherein the editing template of the 3′ PEgRNA and the 5′ PEgRNA each comprises a region of complementarity to each other. For example, the editing template of the 3′ PEgRNA can comprise any of the RTT sequences provided in Table 38. The terms editing template and RTT may be used interchangeably. Each of the RTT sequences in Table 38 is assigned a RTT pairing number that reflects the complementarity relationship between a 5′ RTT and a 3′ RTT. The 3′ PEgRNA can be used in a dual prime editing system that further comprises a 5′ PEgRNA, e.g., a 5′ PEgRNA having a spacer sequence and a corresponding PBS as exemplified in Tables 1A-19B, wherein the 5′ PEgRNA comprises an editing template comprising a RTT sequence having a region of complementarity to the 3′ RTT. The 5′ RTT can comprise any of the RTT sequences provided in Table 37, wherein the 5′ RTT sequence has the same RTT Pairing number as that of the 3′ RTT. The editing template of the 5′ PEgRNA and the 3′ PEgRNA can be completely complementary to each other, wherein the 5′ editing template and the 3′ editing template have the same length and perfect complementarity to each other. Alternatively, the 5′ editing template can comprise a region of complementarity to the 3′ editing template, and further comprise a non-complementary region to the 3′ editing template. The 3′ editing template can comprise a region of complementarity to the 5′ editing template, and further comprise a non-complementary region to the 5′ editing template. The non-complementary region can be at the 5′ end or the 3′ end of the editing templates. Contacting the target DMPK gene with the prime editing system can result in deletion of the sequence between the 5′ PEgRNA nick and the 3′ PEgRNA nick (the IND) and insertion of the region of complementarity between the 5′ editing template and the 3′ editing template (the OD), or insertion of the region of complementarity between the 5′ editing template and the 3′ editing template as well as the non-complementarity regions (the RD), and hence replace the CTG expansion with the sequence of the OD or the RD. Each of the 3′ PEgRNA is assigned a RTT pairing number, and can be used in a dual prime editing composition with a 5′ PEgRNA comprising an RTT having the same RTT pairing number.

In some embodiments, a prime editing composition comprises a 3′ PEgRNA or component thereof exemplified in Tables 24A and 24B and a 5′ PEgRNA, wherein the editing template of the 3′ PEgRNA comprises a region of identity to the spacer of the 5′ PEgRNA. In some embodiments, the editing template of the 5′ PEgRNA comprises a region of identity to the spacer of the 3′ PEgRNA. In some embodiments, the 3′ editing template comprises a region of complementarity to a region on the first strand of the DMPK gene that is directly 3′ to nucleotide 3 of the search target sequence of the 5′ PEgRNA. In some embodiments, the 5′ editing template comprises a region of complementarity to a region on the second strand of the DMPK gene that is directly 3′ to nucleotide 3 of the search target sequence of the 3′ PEgRNA. For example, the extension arm of the 3′ PEgRNA can comprise an editing template comprising the sequence of any of the RTT sequences provided in Table 40. Each of the RTT sequences in Table 40 is assigned a pairing 5′ spacer number. The 3′ PEgRNA can be used in a dual prime editing system with a 5′ PEgRNA, wherein the 5′ PEgRNA comprises a spacer that has the Pairing 5′ spacer number assigned to the 3′ RTT in Table 40. Contacting the target DMPK gene with the prime editing system can result in deletion of the sequence between the 5′ PEgRNA nick and the 3′ PEgRNA nick (the IND) without insertion of any exogenous sequence (a “clean deletion”). Alternatively, the 5′ editing template and the 3′ editing template can further comprise a region of complementarity to each other, and the region of complementarity can replace the IND in the DMPK gene. For example, in some embodiments, the 3′ PEgRNA is part of a dual prime editing composition that further comprises a 5′ PEgRNA, wherein the 3′ PEgRNA comprises a RTT comprising a sequence selected from sequence numbers 3477-3479, and the 5′ PEgRNA comprises a spacer comprising nucleotides 5-20 of sequence number 484. In some embodiments, the 3′ PEgRNA is part of a dual prime editing composition that further comprises a 5′ PEgRNA, wherein the 3′ PEgRNA comprises a RTT comprising a sequence selected from sequence numbers 3480-3484, and the 5′ PEgRNA comprises a spacer comprising nucleotides 5-20 of sequence number 77. In some embodiments, the 3′ PEgRNA is part of a dual prime editing composition that further comprises a 5′ PEgRNA, wherein the 3′ PEgRNA comprises a RTT comprising a sequence selected from sequence numbers 3485-3489, and the 5′ PEgRNA comprises a spacer comprising nucleotides 5-20 of sequence number 590. In some embodiments, the 3′ PEgRNA is part of a dual prime editing composition that further comprises a 5′ PEgRNA, wherein the 3′ PEgRNA comprises a RTT comprising a sequence selected from sequence numbers 3490-3494, and the 5′ PEgRNA comprises a spacer comprising nucleotides 5-20 of sequence number 1019. In some embodiments, the 3′ PEgRNA is part of a dual prime editing composition that further comprises a 5′ PEgRNA, wherein the 3′ PEgRNA comprises a RTT comprising nucleotides x-40 of sequence number 3664, 3665, 3666, 3667, 3668, 3669, 3670, 3671, 3672, 3673, 3674, 3675, 3676, 3677, 3678, 3679, 3680, 3681, or 3682, wherein the 5′ PEgRNA comprises a spacer comprising nucleotides 5-20 of sequence number 518, 536, 554, 572, 590, 39, 912, 929, 947, 965, 983, 1001, 1019, 1341, 20, 77, 484, 58, or 1 respectively in the same order, and wherein x is any integer from 1 to 31. Each 3′ PEgRNA is assigned a RTT pairing number, which corresponds to a pairing 5′ spacer number in Table 40. The 3′ PEgRNA can be used in a dual prime editing system or composition with a 5′ PEgRNA comprising a spacer of the same pairing 5′ spacer number. Preferably, the 5′ PEgRNA comprises a “clean deletion” RTT, for example, an RTT selected from Table 39 that has a Pairing 3′ spacer number that is 5.

### Tables 25A and 25B

Tables 25A and 25B provide exemplary 3′ PEgRNAs and components that can be used with any prime editor containing a Cas9 protein capable of recognizing a NGG PAM sequence, wherein N is A, G, C, or T. The PEgRNAs exemplified in Tables 25A and 25B comprise: (a) a spacer comprising at its 3′ end a sequence corresponding to nucleotides 5-20 of sequence number 1864, (b) a gRNA core capable of binding to a Cas9 protein, and (c) an extension arm comprising (i) an editing template and (ii) a primer binding site (PBS) comprising at its 5′ end the sequence corresponding to sequence number 1879.

The PEgRNA spacer can be, for example, 16-22 nucleotides in length. In some embodiments, the PEgNA spacer is 16-20 nucleotides in length. In some embodiments, the PEgRNA spacer comprises nucleotides 5-20, 4-20, 3-20, 2-20, or 1-20 of sequence number 1864. In some embodiments, the PEgRNA spacer comprises the sequence of sequence number 1864. In some embodiments, the PEgRNA spacer is 20 nucleotides in length. Any of these spacers exemplified can be referred to as 3′ spacer No. 6.

The PBS can be, for example, 3 to 19 nucleotides in length. In some embodiments, the PBS is 3 to 17 nucleotides in length and corresponds to a PBS sequence in Table 20A. In some cases, a PBS length of no more than 3 nucleotides less than the PEgRNA spacer length is chosen.

The 3′ PEgRNAs of Tables 25A and 25B can be used in a dual prime editing system or composition further comprising any suitable 5′ PEgRNA. The prime editing system can be used, e.g., for editing a DMPK gene harboring 50 or more CTG repeats in the DMPK gene.

In some embodiments, a prime editing composition comprises a 3′ PEgRNA or component thereof exemplified in Tables 25A and 25B and a 5′ PEgRNA, wherein the editing template of the 3′ PEgRNA and the 5′ PEgRNA each comprises a region of complementarity to each other. For example, the editing template of the 3′ PEgRNA can comprise any of the RTT sequences provided in Table 38. The terms editing template and RTT may be used interchangeably. Each of the RTT sequences in Table 38 is assigned a RTT pairing number that reflects the complementarity relationship between a 5′ RTT and a 3′ RTT. The 3′ PEgRNA can be used in a dual prime editing system that further comprises a 5′ PEgRNA, e.g., a 5′ PEgRNA having a spacer sequence and a corresponding PBS as exemplified in Tables 1A-19B, wherein the 5′ PEgRNA comprises an editing template comprising a RTT sequence having a region of complementarity to the 3′ RTT. The 5′ RTT can comprise any of the RTT sequences provided in Table 37, wherein the 5′ RTT sequence has the same RTT Pairing number as that of the 3′ RTT. The editing template of the 5′ PEgRNA and the 3′ PEgRNA can be completely complementary to each other, wherein the 5′ editing template and the 3′ editing template have the same length and perfect complementarity to each other. Alternatively, the 5′ editing template can comprise a region of complementarity to the 3′ editing template, and further comprise a non-complementary region to the 3′ editing template. The 3′ editing template can comprise a region of complementarity to the 5′ editing template, and further comprise a non-complementary region to the 5′ editing template. The non-complementary region can be at the 5′ end or the 3′ end of the editing templates. Contacting the target DMPK gene with the prime editing system can result in deletion of the sequence between the 5′ PEgRNA nick and the 3′ PEgRNA nick (the IND) and insertion of the region of complementarity between the 5′ editing template and the 3′ editing template (the OD), or insertion of the region of complementarity between the 5′ editing template and the 3′ editing template as well as the non-complementarity regions (the RD), and hence replace the CTG expansion with the sequence of the OD or the RD. Each of the 3′ PEgRNA is assigned a RTT pairing number, and can be used in a dual prime editing composition with a 5′ PEgRNA comprising an RTT having the same RTT pairing number.

In some embodiments, a prime editing composition comprises a 3′ PEgRNA or component thereof exemplified in Tables 25A and 25B and a 5′ PEgRNA, wherein the editing template of the 3′ PEgRNA comprises a region of identity to the spacer of the 5′ PEgRNA. In some embodiments, the editing template of the 5′ PEgRNA comprises a region of identity to the spacer of the 3′ PEgRNA. In some embodiments, the 3′ editing template comprises a region of complementarity to a region on the first strand of the DMPK gene that is directly 3′ to nucleotide 3 of the search target sequence of the 5′ PEgRNA. In some embodiments, the 5′ editing template comprises a region of complementarity to a region on the second strand of the DMPK gene that is directly 3′ to nucleotide 3 of the search target sequence of the 3′ PEgRNA. For example, the extension arm of the 3′ PEgRNA can comprise an editing template comprising the sequence of any of the RTT sequences provided in Table 40. Each of the RTT sequences in Table 40 is assigned a pairing 5′ spacer number. The 3′ PEgRNA can be used in a dual prime editing system with a 5′ PEgRNA, wherein the 5′ PEgRNA comprises a spacer that has the Pairing 5′ spacer number assigned to the 3′ RTT in Table 40. Contacting the target DMPK gene with the prime editing system can result in deletion of the sequence between the 5′ PEgRNA nick and the 3′ PEgRNA nick (the IND) without insertion of any exogenous sequence (a “clean deletion”). Alternatively, the 5′ editing template and the 3′ editing template can further comprise a region of complementarity to each other, and the region of complementarity can replace the IND in the DMPK gene. For example, in some embodiments, the 3′ PEgRNA is part of a dual prime editing composition that further comprises a 5′ PEgRNA, wherein the 3′ PEgRNA comprises a RTT comprising a sequence selected from sequence numbers 3477-3479, and the 5′ PEgRNA comprises a spacer comprising nucleotides 5-20 of sequence number 484. In some embodiments, the 3′ PEgRNA is part of a dual prime editing composition that further comprises a 5′ PEgRNA, wherein the 3′ PEgRNA comprises a RTT comprising a sequence selected from sequence numbers 3480-3484, and the 5′ PEgRNA comprises a spacer comprising nucleotides 5-20 of sequence number 77. In some embodiments, the 3′ PEgRNA is part of a dual prime editing composition that further comprises a 5′ PEgRNA, wherein the 3′ PEgRNA comprises a RTT comprising a sequence selected from sequence numbers 3485-3489, and the 5′ PEgRNA comprises a spacer comprising nucleotides 5-20 of sequence number 590. In some embodiments, the 3′ PEgRNA is part of a dual prime editing composition that further comprises a 5′ PEgRNA, wherein the 3′ PEgRNA comprises a RTT comprising a sequence selected from sequence numbers 3490-3494, and the 5′ PEgRNA comprises a spacer comprising nucleotides 5-20 of sequence number 1019. In some embodiments, the 3′ PEgRNA is part of a dual prime editing composition that further comprises a 5′ PEgRNA, wherein the 3′ PEgRNA comprises a RTT comprising nucleotides x-40 of sequence number 3664, 3665, 3666, 3667, 3668, 3669, 3670, 3671, 3672, 3673, 3674, 3675, 3676, 3677, 3678, 3679, 3680, 3681, or 3682, wherein the 5′ PEgRNA comprises a spacer comprising nucleotides 5-20 of sequence number 518, 536, 554, 572, 590, 39, 912, 929, 947, 965, 983, 1001, 1019, 1341, 20, 77, 484, 58, or 1 respectively in the same order, and wherein x is any integer from 1 to 31. Each 3′ PEgRNA is assigned a RTT pairing number, which corresponds to a pairing 5′ spacer number in Table 40. The 3′ PEgRNA can be used in a dual prime editing system or composition with a 5′ PEgRNA comprising a spacer of the same pairing 5′ spacer number. Preferably, the 5′ PEgRNA comprises a “clean deletion” RTT, for example, an RTT selected from Table 39 that has a Pairing 3′ spacer number that is 6.

### Tables 26A and 26B

Tables 26A and 26B provide exemplary 3′ PEgRNAs and components that can be used with any prime editor containing a Cas9 protein capable of recognizing a NGG PAM sequence, wherein N is A, G, C, or T. The PEgRNAs exemplified in Tables 26A and 26B comprise: (a) a spacer comprising at its 3′ end a sequence corresponding to nucleotides 5-20 of sequence number 1882, (b) a gRNA core capable of binding to a Cas9 protein, and (c) an extension arm comprising (i) an editing template and (ii) a primer binding site (PBS) comprising at its 5′ end the sequence corresponding to sequence number 1897.

The PEgRNA spacer can be, for example, 16-22 nucleotides in length. In some embodiments, the PEgNA spacer is 16-20 nucleotides in length. In some embodiments, the PEgRNA spacer comprises nucleotides 5-20, 4-20, 3-20, 2-20, or 1-20 of sequence number 1882. In some embodiments, the PEgRNA spacer comprises the sequence of sequence number 1882. In some embodiments, the PEgRNA spacer is 20 nucleotides in length. Any of these spacers exemplified can be referred to as 3′ spacer No. 7.

The PBS can be, for example, 3 to 19 nucleotides in length. In some embodiments, the PBS is 3 to 17 nucleotides in length and corresponds to a PBS sequence in Table 20A. In some cases, a PBS length of no more than 3 nucleotides less than the PEgRNA spacer length is chosen.

The 3′ PEgRNAs of Tables 26A and 26B can be used in a dual prime editing system or composition further comprising any suitable 5′ PEgRNA. The prime editing system can be used, e.g., for editing a DMPK gene harboring 50 or more CTG repeats in the DMPK gene.

In some embodiments, a prime editing composition comprises a 3′ PEgRNA or component thereof exemplified in Tables 26A and 26B and a 5′ PEgRNA, wherein the editing template of the 3′ PEgRNA and the 5′ PEgRNA each comprises a region of complementarity to each other. For example, the editing template of the 3′ PEgRNA can comprise any of the RTT sequences provided in Table 38. The terms editing template and RTT may be used interchangeably. Each of the RTT sequences in Table 38 is assigned a RTT pairing number that reflects the complementarity relationship between a 5′ RTT and a 3′ RTT. The 3′ PEgRNA can be used in a dual prime editing system that further comprises a 5′ PEgRNA, e.g., a 5′ PEgRNA having a spacer sequence and a corresponding PBS as exemplified in Tables 1A-19B, wherein the 5′ PEgRNA comprises an editing template comprising a RTT sequence having a region of complementarity to the 3′ RTT. The 5′ RTT can comprise any of the RTT sequences provided in Table 37, wherein the 5′ RTT sequence has the same RTT Pairing number as that of the 3′ RTT. The editing template of the 5′ PEgRNA and the 3′ PEgRNA can be completely complementary to each other, wherein the 5′ editing template and the 3′ editing template have the same length and perfect complementarity to each other. Alternatively, the 5′ editing template can comprise a region of complementarity to the 3′ editing template, and further comprise a non-complementary region to the 3′ editing template. The 3′ editing template can comprise a region of complementarity to the 5′ editing template, and further comprise a non-complementary region to the 5′ editing template. The non-complementary region can be at the 5′ end or the 3′ end of the editing templates. Contacting the target DMPK gene with the prime editing system can result in deletion of the sequence between the 5′ PEgRNA nick and the 3′ PEgRNA nick (the IND) and insertion of the region of complementarity between the 5′ editing template and the 3′ editing template (the OD), or insertion of the region of complementarity between the 5′ editing template and the 3′ editing template as well as the non-complementarity regions (the RD), and hence replace the CTG expansion with the sequence of the OD or the RD. Each of the 3′ PEgRNA is assigned a RTT pairing number, and can be used in a dual prime editing composition with a 5′ PEgRNA comprising an RTT having the same RTT pairing number.

In some embodiments, a prime editing composition comprises a 3′ PEgRNA or component thereof exemplified in Tables 26A and 26B and a 5′ PEgRNA, wherein the editing template of the 3′ PEgRNA comprises a region of identity to the spacer of the 5′ PEgRNA. In some embodiments, the editing template of the 5′ PEgRNA comprises a region of identity to the spacer of the 3′ PEgRNA. In some embodiments, the 3′ editing template comprises a region of complementarity to a region on the first strand of the DMPK gene that is directly 3′ to nucleotide 3 of the search target sequence of the 5′ PEgRNA. In some embodiments, the 5′ editing template comprises a region of complementarity to a region on the second strand of the DMPK gene that is directly 3′ to nucleotide 3 of the search target sequence of the 3′ PEgRNA. For example, the extension arm of the 3′ PEgRNA can comprise an editing template comprising the sequence of any of the RTT sequences provided in Table 40. Each of the RTT sequences in Table 40 is assigned a pairing 5′ spacer number. The 3′ PEgRNA can be used in a dual prime editing system with a 5′ PEgRNA, wherein the 5′ PEgRNA comprises a spacer that has the Pairing 5′ spacer number assigned to the 3′ RTT in Table 40. Contacting the target DMPK gene with the prime editing system can result in deletion of the sequence between the 5′ PEgRNA nick and the 3′ PEgRNA nick (the IND) without insertion of any exogenous sequence (a “clean deletion”). Alternatively, the 5′ editing template and the 3′ editing template can further comprise a region of complementarity to each other, and the region of complementarity can replace the IND in the DMPK gene. For example, in some embodiments, the 3′ PEgRNA is part of a dual prime editing composition that further comprises a 5′ PEgRNA, wherein the 3′ PEgRNA comprises a RTT comprising a sequence selected from sequence numbers 3477-3479, and the 5′ PEgRNA comprises a spacer comprising nucleotides 5-20 of sequence number 484. In some embodiments, the 3′ PEgRNA is part of a dual prime editing composition that further comprises a 5′ PEgRNA, wherein the 3′ PEgRNA comprises a RTT comprising a sequence selected from sequence numbers 3480-3484, and the 5′ PEgRNA comprises a spacer comprising nucleotides 5-20 of sequence number 77. In some embodiments, the 3′ PEgRNA is part of a dual prime editing composition that further comprises a 5′ PEgRNA, wherein the 3′ PEgRNA comprises a RTT comprising a sequence selected from sequence numbers 3485-3489, and the 5′ PEgRNA comprises a spacer comprising nucleotides 5-20 of sequence number 590. In some embodiments, the 3′ PEgRNA is part of a dual prime editing composition that further comprises a 5′ PEgRNA, wherein the 3′ PEgRNA comprises a RTT comprising a sequence selected from sequence numbers 3490-3494, and the 5′ PEgRNA comprises a spacer comprising nucleotides 5-20 of sequence number 1019. In some embodiments, the 3′ PEgRNA is part of a dual prime editing composition that further comprises a 5′ PEgRNA, wherein the 3′ PEgRNA comprises a RTT comprising nucleotides x-40 of sequence number 3664, 3665, 3666, 3667, 3668, 3669, 3670, 3671, 3672, 3673, 3674, 3675, 3676, 3677, 3678, 3679, 3680, 3681, or 3682, wherein the 5′ PEgRNA comprises a spacer comprising nucleotides 5-20 of sequence number 518, 536, 554, 572, 590, 39, 912, 929, 947, 965, 983, 1001, 1019, 1341, 20, 77, 484, 58, or 1 respectively in the same order, and wherein x is any integer from 1 to 31. Each 3′ PEgRNA is assigned a RTT pairing number, which corresponds to a pairing 5′ spacer number in Table 40. The 3′ PEgRNA can be used in a dual prime editing system or composition with a 5′ PEgRNA comprising a spacer of the same pairing 5′ spacer number. Preferably, the 5′ PEgRNA comprises a “clean deletion” RTT, for example, an RTT selected from Table 39 that has a Pairing 3′ spacer number that is 7.

### Tables 28A and 28B

Tables 28A and 28B provide exemplary 3′ PEgRNAs and components that can be used with any prime editor containing a Cas9 protein capable of recognizing a NGG PAM sequence, wherein N is A, G, C, or T. The PEgRNAs exemplified in Tables 28A and 28B comprise: (a) a spacer comprising at its 3′ end a sequence corresponding to nucleotides 5-20 of sequence number 1918, (b) a gRNA core capable of binding to a Cas9 protein, and (c) an extension arm comprising (i) an editing template and (ii) a primer binding site (PBS) comprising at its 5′ end the sequence corresponding to sequence number 1933.

The PEgRNA spacer can be, for example, 16-22 nucleotides in length. In some embodiments, the PEgNA spacer is 16-20 nucleotides in length. In some embodiments, the PEgRNA spacer comprises nucleotides 5-20, 4-20, 3-20, 2-20, or 1-20 of sequence number 1918. In some embodiments, the PEgRNA spacer comprises the sequence of sequence number 1918. In some embodiments, the PEgRNA spacer is 20 nucleotides in length. Any of these spacers exemplified can be referred to as 3′ spacer No. 9.

The PBS can be, for example, 3 to 19 nucleotides in length. In some embodiments, the PBS is 3 to 17 nucleotides in length and corresponds to a PBS sequence in Table 20A. In some cases, a PBS length of no more than 3 nucleotides less than the PEgRNA spacer length is chosen.

The 3′ PEgRNAs of Tables 28A and 28B can be used in a dual prime editing system or composition further comprising any suitable 5′ PEgRNA. The prime editing system can be used, e.g., for editing a DMPK gene harboring 50 or more CTG repeats in the DMPK gene.

In some embodiments, a prime editing composition comprises a 3′ PEgRNA or component thereof exemplified in Tables 28A and 28B and a 5′ PEgRNA, wherein the editing template of the 3′ PEgRNA and the 5′ PEgRNA each comprises a region of complementarity to each other. For example, the editing template of the 3′ PEgRNA can comprise any of the RTT sequences provided in Table 38. The terms editing template and RTT may be used interchangeably. Each of the RTT sequences in Table 38 is assigned a RTT pairing number that reflects the complementarity relationship between a 5′ RTT and a 3′ RTT. The 3′ PEgRNA can be used in a dual prime editing system that further comprises a 5′ PEgRNA, e.g., a 5′ PEgRNA having a spacer sequence and a corresponding PBS as exemplified in Tables 1A-19B, wherein the 5′ PEgRNA comprises an editing template comprising a RTT sequence having a region of complementarity to the 3′ RTT. The 5′ RTT can comprise any of the RTT sequences provided in Table 37, wherein the 5′ RTT sequence has the same RTT Pairing number as that of the 3′ RTT. The editing template of the 5′ PEgRNA and the 3′ PEgRNA can be completely complementary to each other, wherein the 5′ editing template and the 3′ editing template have the same length and perfect complementarity to each other. Alternatively, the 5′ editing template can comprise a region of complementarity to the 3′ editing template, and further comprise a non-complementary region to the 3′ editing template. The 3′ editing template can comprise a region of complementarity to the 5′ editing template, and further comprise a non-complementary region to the 5′ editing template. The non-complementary region can be at the 5′ end or the 3′ end of the editing templates. Contacting the target DMPK gene with the prime editing system can result in deletion of the sequence between the 5′ PEgRNA nick and the 3′ PEgRNA nick (the IND) and insertion of the region of complementarity between the 5′ editing template and the 3′ editing template (the OD), or insertion of the region of complementarity between the 5′ editing template and the 3′ editing template as well as the non-complementarity regions (the RD), and hence replace the CTG expansion with the sequence of the OD or the RD. Each of the 3′ PEgRNA is assigned a RTT pairing number, and can be used in a dual prime editing composition with a 5′ PEgRNA comprising an RTT having the same RTT pairing number.

In some embodiments, a prime editing composition comprises a 3′ PEgRNA or component thereof exemplified in Tables 28A and 28B and a 5′ PEgRNA, wherein the editing template of the 3′ PEgRNA comprises a region of identity to the spacer of the 5′ PEgRNA. In some embodiments, the editing template of the 5′ PEgRNA comprises a region of identity to the spacer of the 3′ PEgRNA. In some embodiments, the 3′ editing template comprises a region of complementarity to a region on the first strand of the DMPK gene that is directly 3′ to nucleotide 3 of the search target sequence of the 5′ PEgRNA. In some embodiments, the 5′ editing template comprises a region of complementarity to a region on the second strand of the DMPK gene that is directly 3′ to nucleotide 3 of the search target sequence of the 3′ PEgRNA. For example, the extension arm of the 3′ PEgRNA can comprise an editing template comprising the sequence of any of the RTT sequences provided in Table 40. Each of the RTT sequences in Table 40 is assigned a pairing 5′ spacer number. The 3′ PEgRNA can be used in a dual prime editing system with a 5′ PEgRNA, wherein the 5′ PEgRNA comprises a spacer that has the Pairing 5′ spacer number assigned to the 3′ RTT in Table 40. Contacting the target DMPK gene with the prime editing system can result in deletion of the sequence between the 5′ PEgRNA nick and the 3′ PEgRNA nick (the IND) without insertion of any exogenous sequence (a “clean deletion”). Alternatively, the 5′ editing template and the 3′ editing template can further comprise a region of complementarity to each other, and the region of complementarity can replace the IND in the DMPK gene. For example, in some embodiments, the 3′ PEgRNA is part of a dual prime editing composition that further comprises a 5′ PEgRNA, wherein the 3′ PEgRNA comprises a RTT comprising a sequence selected from sequence numbers 3477-3479, and the 5′ PEgRNA comprises a spacer comprising nucleotides 5-20 of sequence number 484. In some embodiments, the 3′ PEgRNA is part of a dual prime editing composition that further comprises a 5′ PEgRNA, wherein the 3′ PEgRNA comprises a RTT comprising a sequence selected from sequence numbers 3480-3484, and the 5′ PEgRNA comprises a spacer comprising nucleotides 5-20 of sequence number 77. In some embodiments, the 3′ PEgRNA is part of a dual prime editing composition that further comprises a 5′ PEgRNA, wherein the 3′ PEgRNA comprises a RTT comprising a sequence selected from sequence numbers 3485-3489, and the 5′ PEgRNA comprises a spacer comprising nucleotides 5-20 of sequence number 590. In some embodiments, the 3′ PEgRNA is part of a dual prime editing composition that further comprises a 5′ PEgRNA, wherein the 3′ PEgRNA comprises a RTT comprising a sequence selected from sequence numbers 3490-3494, and the 5′ PEgRNA comprises a spacer comprising nucleotides 5-20 of sequence number 1019. In some embodiments, the 3′ PEgRNA is part of a dual prime editing composition that further comprises a 5′ PEgRNA, wherein the 3′ PEgRNA comprises a RTT comprising nucleotides x-40 of sequence number 3664, 3665, 3666, 3667, 3668, 3669, 3670, 3671, 3672, 3673, 3674, 3675, 3676, 3677, 3678, 3679, 3680, 3681, or 3682, wherein the 5′ PEgRNA comprises a spacer comprising nucleotides 5-20 of sequence number 518, 536, 554, 572, 590, 39, 912, 929, 947, 965, 983, 1001, 1019, 1341, 20, 77, 484, 58, or 1 respectively in the same order, and wherein x is any integer from 1 to 31. Each 3′ PEgRNA is assigned a RTT pairing number, which corresponds to a pairing 5′ spacer number in Table 40. The 3′ PEgRNA can be used in a dual prime editing system or composition with a 5′ PEgRNA comprising a spacer of the same pairing 5′ spacer number. Preferably, the 5′ PEgRNA comprises a “clean deletion” RTT, for example, an RTT selected from Table 39 that has a Pairing 3′ spacer number that is 9.

### Tables 30A and 30B

Tables 30A and 30B provide exemplary 3′ PEgRNAs and components that can be used with any prime editor containing a Cas9 protein capable of recognizing a NGG PAM sequence, wherein N is A, G, C, or T. The PEgRNAs exemplified in Tables 30A and 30B comprise: (a) a spacer comprising at its 3′ end a sequence corresponding to nucleotides 5-20 of sequence number 2263, (b) a gRNA core capable of binding to a Cas9 protein, and (c) an extension arm comprising (i) an editing template and (ii) a primer binding site (PBS) comprising at its 5′ end the sequence corresponding to sequence number 2278.

The PEgRNA spacer can be, for example, 16-22 nucleotides in length. In some embodiments, the PEgNA spacer is 16-20 nucleotides in length. In some embodiments, the PEgRNA spacer comprises nucleotides 5-20, 4-20, 3-20, 2-20, or 1-20 of sequence number 2263. In some embodiments, the PEgRNA spacer comprises the sequence of sequence number 2263. In some embodiments, the PEgRNA spacer is 20 nucleotides in length. Any of these spacers exemplified can be referred to as 3′ spacer No. 11.

The PBS can be, for example, 3 to 19 nucleotides in length. In some embodiments, the PBS is 3 to 17 nucleotides in length and corresponds to a PBS sequence in Table 20A. In some cases, a PBS length of no more than 3 nucleotides less than the PEgRNA spacer length is chosen.

The 3′ PEgRNAs of Tables 30A and 30B can be used in a dual prime editing system or composition further comprising any suitable 5′ PEgRNA. The prime editing system can be used, e.g., for editing a DMPK gene harboring 50 or more CTG repeats in the DMPK gene.

In some embodiments, a prime editing composition comprises a 3′ PEgRNA or component thereof exemplified in Tables 30A and 30B and a 5′ PEgRNA, wherein the editing template of the 3′ PEgRNA and the 5′ PEgRNA each comprises a region of complementarity to each other. For example, the editing template of the 3′ PEgRNA can comprise any of the RTT sequences provided in Table 38. The terms editing template and RTT may be used interchangeably. Each of the RTT sequences in Table 38 is assigned a RTT pairing number that reflects the complementarity relationship between a 5′ RTT and a 3′ RTT. The 3′ PEgRNA can be used in a dual prime editing system that further comprises a 5′ PEgRNA, e.g., a 5′ PEgRNA having a spacer sequence and a corresponding PBS as exemplified in Tables 1A-19B, wherein the 5′ PEgRNA comprises an editing template comprising a RTT sequence having a region of complementarity to the 3′ RTT. The 5′ RTT can comprise any of the RTT sequences provided in Table 37, wherein the 5′ RTT sequence has the same RTT Pairing number as that of the 3′ RTT. The editing template of the 5′ PEgRNA and the 3′ PEgRNA can be completely complementary to each other, wherein the 5′ editing template and the 3′ editing template have the same length and perfect complementarity to each other. Alternatively, the 5′ editing template can comprise a region of complementarity to the 3′ editing template, and further comprise a non-complementary region to the 3′ editing template. The 3′ editing template can comprise a region of complementarity to the 5′ editing template, and further comprise a non-complementary region to the 5′ editing template. The non-complementary region can be at the 5′ end or the 3′ end of the editing templates. Contacting the target DMPK gene with the prime editing system can result in deletion of the sequence between the 5′ PEgRNA nick and the 3′ PEgRNA nick (the IND) and insertion of the region of complementarity between the 5′ editing template and the 3′ editing template (the OD), or insertion of the region of complementarity between the 5′ editing template and the 3′ editing template as well as the non-complementarity regions (the RD), and hence replace the CTG expansion with the sequence of the OD or the RD. Each of the 3′ PEgRNA is assigned a RTT pairing number, and can be used in a dual prime editing composition with a 5′ PEgRNA comprising an RTT having the same RTT pairing number.

In some embodiments, a prime editing composition comprises a 3′ PEgRNA or component thereof exemplified in Tables 30A and 30B and a 5′ PEgRNA, wherein the editing template of the 3′ PEgRNA comprises a region of identity to the spacer of the 5′ PEgRNA. In some embodiments, the editing template of the 5′ PEgRNA comprises a region of identity to the spacer of the 3′ PEgRNA. In some embodiments, the 3′ editing template comprises a region of complementarity to a region on the first strand of the DMPK gene that is directly 3′ to nucleotide 3 of the search target sequence of the 5′ PEgRNA. In some embodiments, the 5′ editing template comprises a region of complementarity to a region on the second strand of the DMPK gene that is directly 3′ to nucleotide 3 of the search target sequence of the 3′ PEgRNA. For example, the extension arm of the 3′ PEgRNA can comprise an editing template comprising the sequence of any of the RTT sequences provided in Table 40. Each of the RTT sequences in Table 40 is assigned a pairing 5′ spacer number. The 3′ PEgRNA can be used in a dual prime editing system with a 5′ PEgRNA, wherein the 5′ PEgRNA comprises a spacer that has the Pairing 5′ spacer number assigned to the 3′ RTT in Table 40. Contacting the target DMPK gene with the prime editing system can result in deletion of the sequence between the 5′ PEgRNA nick and the 3′ PEgRNA nick (the IND) without insertion of any exogenous sequence (a “clean deletion”). Alternatively, the 5′ editing template and the 3′ editing template can further comprise a region of complementarity to each other, and the region of complementarity can replace the IND in the DMPK gene. For example, in some embodiments, the 3′ PEgRNA is part of a dual prime editing composition that further comprises a 5′ PEgRNA, wherein the 3′ PEgRNA comprises a RTT comprising a sequence selected from sequence numbers 3477-3479, and the 5′ PEgRNA comprises a spacer comprising nucleotides 5-20 of sequence number 484. In some embodiments, the 3′ PEgRNA is part of a dual prime editing composition that further comprises a 5′ PEgRNA, wherein the 3′ PEgRNA comprises a RTT comprising a sequence selected from sequence numbers 3480-3484, and the 5′ PEgRNA comprises a spacer comprising nucleotides 5-20 of sequence number 77. In some embodiments, the 3′ PEgRNA is part of a dual prime editing composition that further comprises a 5′ PEgRNA, wherein the 3′ PEgRNA comprises a RTT comprising a sequence selected from sequence numbers 3485-3489, and the 5′ PEgRNA comprises a spacer comprising nucleotides 5-20 of sequence number 590. In some embodiments, the 3′ PEgRNA is part of a dual prime editing composition that further comprises a 5′ PEgRNA, wherein the 3′ PEgRNA comprises a RTT comprising a sequence selected from sequence numbers 3490-3494, and the 5′ PEgRNA comprises a spacer comprising nucleotides 5-20 of sequence number 1019. In some embodiments, the 3′ PEgRNA is part of a dual prime editing composition that further comprises a 5′ PEgRNA, wherein the 3′ PEgRNA comprises a RTT comprising nucleotides x-40 of sequence number 3664, 3665, 3666, 3667, 3668, 3669, 3670, 3671, 3672, 3673, 3674, 3675, 3676, 3677, 3678, 3679, 3680, 3681, or 3682, wherein the 5′ PEgRNA comprises a spacer comprising nucleotides 5-20 of sequence number 518, 536, 554, 572, 590, 39, 912, 929, 947, 965, 983, 1001, 1019, 1341, 20, 77, 484, 58, or 1 respectively in the same order, and wherein x is any integer from 1 to 31. Each 3′ PEgRNA is assigned a RTT pairing number, which corresponds to a pairing 5′ spacer number in Table 40. The 3′ PEgRNA can be used in a dual prime editing system or composition with a 5′ PEgRNA comprising a spacer of the same pairing 5′ spacer number. Preferably, the 5′ PEgRNA comprises a “clean deletion” RTT, for example, an RTT selected from Table 39 that has a Pairing 3′ spacer number that is 11.

### Tables 31A and 31B

Tables 31A and 31B provide exemplary 3′ PEgRNAs and components that can be used with any prime editor containing a Cas9 protein capable of recognizing a NGG PAM sequence, wherein N is A, G, C, or T. The PEgRNAs exemplified in Tables 31A and 31B comprise: (a) a spacer comprising at its 3′ end a sequence corresponding to nucleotides 5-20 of sequence number 2281, (b) a gRNA core capable of binding to a Cas9 protein, and (c) an extension arm comprising (i) an editing template and (ii) a primer binding site (PBS) comprising at its 5′ end the sequence corresponding to sequence number 2296.

The PEgRNA spacer can be, for example, 16-22 nucleotides in length. In some embodiments, the PEgNA spacer is 16-20 nucleotides in length. In some embodiments, the PEgRNA spacer comprises nucleotides 5-20, 4-20, 3-20, 2-20, or 1-20 of sequence number 2281. In some embodiments, the PEgRNA spacer comprises the sequence of sequence number 2281. In some embodiments, the PEgRNA spacer is 20 nucleotides in length. Any of these spacers exemplified can be referred to as 3′ spacer No. 12.

The PBS can be, for example, 3 to 19 nucleotides in length. In some embodiments, the PBS is 3 to 17 nucleotides in length and corresponds to a PBS sequence in Table 20A. In some cases, a PBS length of no more than 3 nucleotides less than the PEgRNA spacer length is chosen.

The 3′ PEgRNAs of Tables 31A and 31B can be used in a dual prime editing system or composition further comprising any suitable 5′ PEgRNA. The prime editing system can be used, e.g., for editing a DMPK gene harboring 50 or more CTG repeats in the DMPK gene.

In some embodiments, a prime editing composition comprises a 3′ PEgRNA or component thereof exemplified in Tables 31A and 31B and a 5′ PEgRNA, wherein the editing template of the 3′ PEgRNA and the 5′ PEgRNA each comprises a region of complementarity to each other. For example, the editing template of the 3′ PEgRNA can comprise any of the RTT sequences provided in Table 38. The terms editing template and RTT may be used interchangeably. Each of the RTT sequences in Table 38 is assigned a RTT pairing number that reflects the complementarity relationship between a 5′ RTT and a 3′ RTT. The 3′ PEgRNA can be used in a dual prime editing system that further comprises a 5′ PEgRNA, e.g., a 5′ PEgRNA having a spacer sequence and a corresponding PBS as exemplified in Tables 1A-19B, wherein the 5′ PEgRNA comprises an editing template comprising a RTT sequence having a region of complementarity to the 3′ RTT. The 5′ RTT can comprise any of the RTT sequences provided in Table 37, wherein the 5′ RTT sequence has the same RTT Pairing number as that of the 3′ RTT. The editing template of the 5′ PEgRNA and the 3′ PEgRNA can be completely complementary to each other, wherein the 5′ editing template and the 3′ editing template have the same length and perfect complementarity to each other. Alternatively, the 5′ editing template can comprise a region of complementarity to the 3′ editing template, and further comprise a non-complementary region to the 3′ editing template. The 3′ editing template can comprise a region of complementarity to the 5′ editing template, and further comprise a non-complementary region to the 5′ editing template. The non-complementary region can be at the 5′ end or the 3′ end of the editing templates. Contacting the target DMPK gene with the prime editing system can result in deletion of the sequence between the 5′ PEgRNA nick and the 3′ PEgRNA nick (the IND) and insertion of the region of complementarity between the 5′ editing template and the 3′ editing template (the OD), or insertion of the region of complementarity between the 5′ editing template and the 3′ editing template as well as the non-complementarity regions (the RD), and hence replace the CTG expansion with the sequence of the OD or the RD. Each of the 3′ PEgRNA is assigned a RTT pairing number, and can be used in a dual prime editing composition with a 5′ PEgRNA comprising an RTT having the same RTT pairing number.

In some embodiments, a prime editing composition comprises a 3′ PEgRNA or component thereof exemplified in Tables 31A and 31B and a 5′ PEgRNA, wherein the editing template of the 3′ PEgRNA comprises a region of identity to the spacer of the 5′ PEgRNA. In some embodiments, the editing template of the 5′ PEgRNA comprises a region of identity to the spacer of the 3′ PEgRNA. In some embodiments, the 3′ editing template comprises a region of complementarity to a region on the first strand of the DMPK gene that is directly 3′ to nucleotide 3 of the search target sequence of the 5′ PEgRNA. In some embodiments, the 5′ editing template comprises a region of complementarity to a region on the second strand of the DMPK gene that is directly 3′ to nucleotide 3 of the search target sequence of the 3′ PEgRNA. For example, the extension arm of the 3′ PEgRNA can comprise an editing template comprising the sequence of any of the RTT sequences provided in Table 40. Each of the RTT sequences in Table 40 is assigned a pairing 5′ spacer number. The 3′ PEgRNA can be used in a dual prime editing system with a 5′ PEgRNA, wherein the 5′ PEgRNA comprises a spacer that has the Pairing 5′ spacer number assigned to the 3′ RTT in Table 40. Contacting the target DMPK gene with the prime editing system can result in deletion of the sequence between the 5′ PEgRNA nick and the 3′ PEgRNA nick (the IND) without insertion of any exogenous sequence (a “clean deletion”). Alternatively, the 5′ editing template and the 3′ editing template can further comprise a region of complementarity to each other, and the region of complementarity can replace the IND in the DMPK gene. For example, in some embodiments, the 3′ PEgRNA is part of a dual prime editing composition that further comprises a 5′ PEgRNA, wherein the 3′ PEgRNA comprises a RTT comprising a sequence selected from sequence numbers 3477-3479, and the 5′ PEgRNA comprises a spacer comprising nucleotides 5-20 of sequence number 484. In some embodiments, the 3′ PEgRNA is part of a dual prime editing composition that further comprises a 5′ PEgRNA, wherein the 3′ PEgRNA comprises a RTT comprising a sequence selected from sequence numbers 3480-3484, and the 5′ PEgRNA comprises a spacer comprising nucleotides 5-20 of sequence number 77. In some embodiments, the 3′ PEgRNA is part of a dual prime editing composition that further comprises a 5′ PEgRNA, wherein the 3′ PEgRNA comprises a RTT comprising a sequence selected from sequence numbers 3485-3489, and the 5′ PEgRNA comprises a spacer comprising nucleotides 5-20 of sequence number 590. In some embodiments, the 3′ PEgRNA is part of a dual prime editing composition that further comprises a 5′ PEgRNA, wherein the 3′ PEgRNA comprises a RTT comprising a sequence selected from sequence numbers 3490-3494, and the 5′ PEgRNA comprises a spacer comprising nucleotides 5-20 of sequence number 1019. In some embodiments, the 3′ PEgRNA is part of a dual prime editing composition that further comprises a 5′ PEgRNA, wherein the 3′ PEgRNA comprises a RTT comprising nucleotides x-40 of sequence number 3664, 3665, 3666, 3667, 3668, 3669, 3670, 3671, 3672, 3673, 3674, 3675, 3676, 3677, 3678, 3679, 3680, 3681, or 3682, wherein the 5′ PEgRNA comprises a spacer comprising nucleotides 5-20 of sequence number 518, 536, 554, 572, 590, 39, 912, 929, 947, 965, 983, 1001, 1019, 1341, 20, 77, 484, 58, or 1 respectively in the same order, and wherein x is any integer from 1 to 31. Each 3′ PEgRNA is assigned a RTT pairing number, which corresponds to a pairing 5′ spacer number in Table 40. The 3′ PEgRNA can be used in a dual prime editing system or composition with a 5′ PEgRNA comprising a spacer of the same pairing 5′ spacer number. Preferably, the 5′ PEgRNA comprises a “clean deletion” RTT, for example, an RTT selected from Table 39 that has a Pairing 3′ spacer number that is 12.

### Tables 32A and 32B

Tables 32A and 32B provide exemplary 3′ PEgRNAs and components that can be used with any prime editor containing a Cas9 protein capable of recognizing a NGG PAM sequence, wherein N is A, G, C, or T. The PEgRNAs exemplified in Tables 32A and 32B comprise: (a) a spacer comprising at its 3′ end a sequence corresponding to nucleotides 5-20 of sequence number 2299, (b) a gRNA core capable of binding to a Cas9 protein, and (c) an extension arm comprising (i) an editing template and (ii) a primer binding site (PBS) comprising at its 5′ end the sequence corresponding to sequence number 2314.

The PEgRNA spacer can be, for example, 16-22 nucleotides in length. In some embodiments, the PEgNA spacer is 16-20 nucleotides in length. In some embodiments, the PEgRNA spacer comprises nucleotides 5-20, 4-20, 3-20, 2-20, or 1-20 of sequence number 2299. In some embodiments, the PEgRNA spacer comprises the sequence of sequence number 2299. In some embodiments, the PEgRNA spacer is 20 nucleotides in length. Any of these spacers exemplified can be referred to as 3′ spacer No. 13.

The PBS can be, for example, 3 to 19 nucleotides in length. In some embodiments, the PBS is 3 to 17 nucleotides in length and corresponds to a PBS sequence in Table 20A. In some cases, a PBS length of no more than 3 nucleotides less than the PEgRNA spacer length is chosen.

The 3′ PEgRNAs of Tables 32A and 32B can be used in a dual prime editing system or composition further comprising any suitable 5′ PEgRNA. The prime editing system can be used, e.g., for editing a DMPK gene harboring 50 or more CTG repeats in the DMPK gene.

In some embodiments, a prime editing composition comprises a 3′ PEgRNA or component thereof exemplified in Tables 32A and 32B and a 5′ PEgRNA, wherein the editing template of the 3′ PEgRNA and the 5′ PEgRNA each comprises a region of complementarity to each other. For example, the editing template of the 3′ PEgRNA can comprise any of the RTT sequences provided in Table 38. The terms editing template and RTT may be used interchangeably. Each of the RTT sequences in Table 38 is assigned a RTT pairing number that reflects the complementarity relationship between a 5′ RTT and a 3′ RTT. The 3′ PEgRNA can be used in a dual prime editing system that further comprises a 5′ PEgRNA, e.g., a 5′ PEgRNA having a spacer sequence and a corresponding PBS as exemplified in Tables 1A-19B, wherein the 5′ PEgRNA comprises an editing template comprising a RTT sequence having a region of complementarity to the 3′ RTT. The 5′ RTT can comprise any of the RTT sequences provided in Table 37, wherein the 5′ RTT sequence has the same RTT Pairing number as that of the 3′ RTT. The editing template of the 5′ PEgRNA and the 3′ PEgRNA can be completely complementary to each other, wherein the 5′ editing template and the 3′ editing template have the same length and perfect complementarity to each other. Alternatively, the 5′ editing template can comprise a region of complementarity to the 3′ editing template, and further comprise a non-complementary region to the 3′ editing template. The 3′ editing template can comprise a region of complementarity to the 5′ editing template, and further comprise a non-complementary region to the 5′ editing template. The non-complementary region can be at the 5′ end or the 3′ end of the editing templates. Contacting the target DMPK gene with the prime editing system can result in deletion of the sequence between the 5′ PEgRNA nick and the 3′ PEgRNA nick (the IND) and insertion of the region of complementarity between the 5′ editing template and the 3′ editing template (the OD), or insertion of the region of complementarity between the 5′ editing template and the 3′ editing template as well as the non-complementarity regions (the RD), and hence replace the CTG expansion with the sequence of the OD or the RD. Each of the 3′ PEgRNA is assigned a RTT pairing number, and can be used in a dual prime editing composition with a 5′ PEgRNA comprising an RTT having the same RTT pairing number.

In some embodiments, a prime editing composition comprises a 3′ PEgRNA or component thereof exemplified in Tables 32A and 32B and a 5′ PEgRNA, wherein the editing template of the 3′ PEgRNA comprises a region of identity to the spacer of the 5′ PEgRNA. In some embodiments, the editing template of the 5′ PEgRNA comprises a region of identity to the spacer of the 3′ PEgRNA. In some embodiments, the 3′ editing template comprises a region of complementarity to a region on the first strand of the DMPK gene that is directly 3′ to nucleotide 3 of the search target sequence of the 5′ PEgRNA. In some embodiments, the 5′ editing template comprises a region of complementarity to a region on the second strand of the DMPK gene that is directly 3′ to nucleotide 3 of the search target sequence of the 3′ PEgRNA. For example, the extension arm of the 3′ PEgRNA can comprise an editing template comprising the sequence of any of the RTT sequences provided in Table 40. Each of the RTT sequences in Table 40 is assigned a pairing 5′ spacer number. The 3′ PEgRNA can be used in a dual prime editing system with a 5′ PEgRNA, wherein the 5′ PEgRNA comprises a spacer that has the Pairing 5′ spacer number assigned to the 3′ RTT in Table 40. Contacting the target DMPK gene with the prime editing system can result in deletion of the sequence between the 5′ PEgRNA nick and the 3′ PEgRNA nick (the IND) without insertion of any exogenous sequence (a “clean deletion”). Alternatively, the 5′ editing template and the 3′ editing template can further comprise a region of complementarity to each other, and the region of complementarity can replace the IND in the DMPK gene. For example, in some embodiments, the 3′ PEgRNA is part of a dual prime editing composition that further comprises a 5′ PEgRNA, wherein the 3′ PEgRNA comprises a RTT comprising a sequence selected from sequence numbers 3477-3479, and the 5′ PEgRNA comprises a spacer comprising nucleotides 5-20 of sequence number 484. In some embodiments, the 3′ PEgRNA is part of a dual prime editing composition that further comprises a 5′ PEgRNA, wherein the 3′ PEgRNA comprises a RTT comprising a sequence selected from sequence numbers 3480-3484, and the 5′ PEgRNA comprises a spacer comprising nucleotides 5-20 of sequence number 77. In some embodiments, the 3′ PEgRNA is part of a dual prime editing composition that further comprises a 5′ PEgRNA, wherein the 3′ PEgRNA comprises a RTT comprising a sequence selected from sequence numbers 3485-3489, and the 5′ PEgRNA comprises a spacer comprising nucleotides 5-20 of sequence number 590. In some embodiments, the 3′ PEgRNA is part of a dual prime editing composition that further comprises a 5′ PEgRNA, wherein the 3′ PEgRNA comprises a RTT comprising a sequence selected from sequence numbers 3490-3494, and the 5′ PEgRNA comprises a spacer comprising nucleotides 5-20 of sequence number 1019. In some embodiments, the 3′ PEgRNA is part of a dual prime editing composition that further comprises a 5′ PEgRNA, wherein the 3′ PEgRNA comprises a RTT comprising nucleotides x-40 of sequence number 3664, 3665, 3666, 3667, 3668, 3669, 3670, 3671, 3672, 3673, 3674, 3675, 3676, 3677, 3678, 3679, 3680, 3681, or 3682, wherein the 5′ PEgRNA comprises a spacer comprising nucleotides 5-20 of sequence number 518, 536, 554, 572, 590, 39, 912, 929, 947, 965, 983, 1001, 1019, 1341, 20, 77, 484, 58, or 1 respectively in the same order, and wherein x is any integer from 1 to 31. Each 3′ PEgRNA is assigned a RTT pairing number, which corresponds to a pairing 5′ spacer number in Table 40. The 3′ PEgRNA can be used in a dual prime editing system or composition with a 5′ PEgRNA comprising a spacer of the same pairing 5′ spacer number. Preferably, the 5′ PEgRNA comprises a “clean deletion” RTT, for example, an RTT selected from Table 39 that has a Pairing 3′ spacer number that is 13.

### Tables 33A and 33B

Tables 33A and 33B provide exemplary 3′ PEgRNAs and components that can be used with any prime editor containing a Cas9 protein capable of recognizing a NGG PAM sequence, wherein N is A, G, C, or T. The PEgRNAs exemplified in Tables 33A and 33B comprise: (a) a spacer comprising at its 3′ end a sequence corresponding to nucleotides 5-20 of sequence number 2317, (b) a gRNA core capable of binding to a Cas9 protein, and (c) an extension arm comprising (i) an editing template and (ii) a primer binding site (PBS) comprising at its 5′ end the sequence corresponding to sequence number 2332.

The PEgRNA spacer can be, for example, 16-22 nucleotides in length. In some embodiments, the PEgNA spacer is 16-20 nucleotides in length. In some embodiments, the PEgRNA spacer comprises nucleotides 5-20, 4-20, 3-20, 2-20, or 1-20 of sequence number 2317. In some embodiments, the PEgRNA spacer comprises the sequence of sequence number 2317. In some embodiments, the PEgRNA spacer is 20 nucleotides in length. Any of these spacers exemplified can be referred to as 3′ spacer No. 14.

The PBS can be, for example, 3 to 19 nucleotides in length. In some embodiments, the PBS is 3 to 17 nucleotides in length and corresponds to a PBS sequence in Table 20A. In some cases, a PBS length of no more than 3 nucleotides less than the PEgRNA spacer length is chosen.

The 3′ PEgRNAs of Tables 33A and 33B can be used in a dual prime editing system or composition further comprising any suitable 5′ PEgRNA. The prime editing system can be used, e.g., for editing a DMPK gene harboring 50 or more CTG repeats in the DMPK gene.

In some embodiments, a prime editing composition comprises a 3′ PEgRNA or component thereof exemplified in Tables 33A and 33B and a 5′ PEgRNA, wherein the editing template of the 3′ PEgRNA and the 5′ PEgRNA each comprises a region of complementarity to each other. For example, the editing template of the 3′ PEgRNA can comprise any of the RTT sequences provided in Table 38. The terms editing template and RTT may be used interchangeably. Each of the RTT sequences in Table 38 is assigned a RTT pairing number that reflects the complementarity relationship between a 5′ RTT and a 3′ RTT. The 3′ PEgRNA can be used in a dual prime editing system that further comprises a 5′ PEgRNA, e.g., a 5′ PEgRNA having a spacer sequence and a corresponding PBS as exemplified in Tables 1A-19B, wherein the 5′ PEgRNA comprises an editing template comprising a RTT sequence having a region of complementarity to the 3′ RTT. The 5′ RTT can comprise any of the RTT sequences provided in Table 37, wherein the 5′ RTT sequence has the same RTT Pairing number as that of the 3′ RTT. The editing template of the 5′ PEgRNA and the 3′ PEgRNA can be completely complementary to each other, wherein the 5′ editing template and the 3′ editing template have the same length and perfect complementarity to each other. Alternatively, the 5′ editing template can comprise a region of complementarity to the 3′ editing template, and further comprise a non-complementary region to the 3′ editing template. The 3′ editing template can comprise a region of complementarity to the 5′ editing template, and further comprise a non-complementary region to the 5′ editing template. The non-complementary region can be at the 5′ end or the 3′ end of the editing templates. Contacting the target DMPK gene with the prime editing system can result in deletion of the sequence between the 5′ PEgRNA nick and the 3′ PEgRNA nick (the IND) and insertion of the region of complementarity between the 5′ editing template and the 3′ editing template (the OD), or insertion of the region of complementarity between the 5′ editing template and the 3′ editing template as well as the non-complementarity regions (the RD), and hence replace the CTG expansion with the sequence of the OD or the RD. Each of the 3′ PEgRNA is assigned a RTT pairing number, and can be used in a dual prime editing composition with a 5′ PEgRNA comprising an RTT having the same RTT pairing number.

In some embodiments, a prime editing composition comprises a 3′ PEgRNA or component thereof exemplified in Tables 33A and 33B and a 5′ PEgRNA, wherein the editing template of the 3′ PEgRNA comprises a region of identity to the spacer of the 5′ PEgRNA. In some embodiments, the editing template of the 5′ PEgRNA comprises a region of identity to the spacer of the 3′ PEgRNA. In some embodiments, the 3′ editing template comprises a region of complementarity to a region on the first strand of the DMPK gene that is directly 3′ to nucleotide 3 of the search target sequence of the 5′ PEgRNA. In some embodiments, the 5′ editing template comprises a region of complementarity to a region on the second strand of the DMPK gene that is directly 3′ to nucleotide 3 of the search target sequence of the 3′ PEgRNA. For example, the extension arm of the 3′ PEgRNA can comprise an editing template comprising the sequence of any of the RTT sequences provided in Table 40. Each of the RTT sequences in Table 40 is assigned a pairing 5′ spacer number. The 3′ PEgRNA can be used in a dual prime editing system with a 5′ PEgRNA, wherein the 5′ PEgRNA comprises a spacer that has the Pairing 5′ spacer number assigned to the 3′ RTT in Table 40. Contacting the target DMPK gene with the prime editing system can result in deletion of the sequence between the 5′ PEgRNA nick and the 3′ PEgRNA nick (the IND) without insertion of any exogenous sequence (a “clean deletion”). Alternatively, the 5′ editing template and the 3′ editing template can further comprise a region of complementarity to each other, and the region of complementarity can replace the IND in the DMPK gene. For example, in some embodiments, the 3′ PEgRNA is part of a dual prime editing composition that further comprises a 5′ PEgRNA, wherein the 3′ PEgRNA comprises a RTT comprising a sequence selected from sequence numbers 3477-3479, and the 5′ PEgRNA comprises a spacer comprising nucleotides 5-20 of sequence number 484. In some embodiments, the 3′ PEgRNA is part of a dual prime editing composition that further comprises a 5′ PEgRNA, wherein the 3′ PEgRNA comprises a RTT comprising a sequence selected from sequence numbers 3480-3484, and the 5′ PEgRNA comprises a spacer comprising nucleotides 5-20 of sequence number 77. In some embodiments, the 3′ PEgRNA is part of a dual prime editing composition that further comprises a 5′ PEgRNA, wherein the 3′ PEgRNA comprises a RTT comprising a sequence selected from sequence numbers 3485-3489, and the 5′ PEgRNA comprises a spacer comprising nucleotides 5-20 of sequence number 590. In some embodiments, the 3′ PEgRNA is part of a dual prime editing composition that further comprises a 5′ PEgRNA, wherein the 3′ PEgRNA comprises a RTT comprising a sequence selected from sequence numbers 3490-3494, and the 5′ PEgRNA comprises a spacer comprising nucleotides 5-20 of sequence number 1019. In some embodiments, the 3′ PEgRNA is part of a dual prime editing composition that further comprises a 5′ PEgRNA, wherein the 3′ PEgRNA comprises a RTT comprising nucleotides x-40 of sequence number 3664, 3665, 3666, 3667, 3668, 3669, 3670, 3671, 3672, 3673, 3674, 3675, 3676, 3677, 3678, 3679, 3680, 3681, or 3682, wherein the 5′ PEgRNA comprises a spacer comprising nucleotides 5-20 of sequence number 518, 536, 554, 572, 590, 39, 912, 929, 947, 965, 983, 1001, 1019, 1341, 20, 77, 484, 58, or 1 respectively in the same order, and wherein x is any integer from 1 to 31. Each 3′ PEgRNA is assigned a RTT pairing number, which corresponds to a pairing 5′ spacer number in Table 40. The 3′ PEgRNA can be used in a dual prime editing system or composition with a 5′ PEgRNA comprising a spacer of the same pairing 5′ spacer number. Preferably, the 5′ PEgRNA comprises a “clean deletion” RTT, for example, an RTT selected from Table 39 that has a Pairing 3′ spacer number that is 14.

### Tables 34A and 34B

Tables 34A and 34B provide exemplary 3′ PEgRNAs and components that can be used with any prime editor containing a Cas9 protein capable of recognizing a NGG PAM sequence, wherein N is A, G, C, or T. The PEgRNAs exemplified in Tables 34A and 34B comprise: (a) a spacer comprising at its 3′ end a sequence corresponding to nucleotides 5-20 of sequence number 2335, (b) a gRNA core capable of binding to a Cas9 protein, and (c) an extension arm comprising (i) an editing template and (ii) a primer binding site (PBS) comprising at its 5′ end the sequence corresponding to sequence number 2350.

The PEgRNA spacer can be, for example, 16-22 nucleotides in length. In some embodiments, the PEgNA spacer is 16-20 nucleotides in length. In some embodiments, the PEgRNA spacer comprises nucleotides 5-20, 4-20, 3-20, 2-20, or 1-20 of sequence number 2335. In some embodiments, the PEgRNA spacer comprises the sequence of sequence number 2335. In some embodiments, the PEgRNA spacer is 20 nucleotides in length. Any of these spacers exemplified can be referred to as 3′ spacer No. 15.

The PBS can be, for example, 3 to 19 nucleotides in length. In some embodiments, the PBS is 3 to 17 nucleotides in length and corresponds to a PBS sequence in Table 20A. In some cases, a PBS length of no more than 3 nucleotides less than the PEgRNA spacer length is chosen.

The 3′ PEgRNAs of Tables 34A and 34B can be used in a dual prime editing system or composition further comprising any suitable 5′ PEgRNA. The prime editing system can be used, e.g., for editing a DMPK gene harboring 50 or more CTG repeats in the DMPK gene.

In some embodiments, a prime editing composition comprises a 3′ PEgRNA or component thereof exemplified in Tables 34A and 34B and a 5′ PEgRNA, wherein the editing template of the 3′ PEgRNA and the 5′ PEgRNA each comprises a region of complementarity to each other. For example, the editing template of the 3′ PEgRNA can comprise any of the RTT sequences provided in Table 38. The terms editing template and RTT may be used interchangeably. Each of the RTT sequences in Table 38 is assigned a RTT pairing number that reflects the complementarity relationship between a 5′ RTT and a 3′ RTT. The 3′ PEgRNA can be used in a dual prime editing system that further comprises a 5′ PEgRNA, e.g., a 5′ PEgRNA having a spacer sequence and a corresponding PBS as exemplified in Tables 1A-19B, wherein the 5′ PEgRNA comprises an editing template comprising a RTT sequence having a region of complementarity to the 3′ RTT. The 5′ RTT can comprise any of the RTT sequences provided in Table 37, wherein the 5′ RTT sequence has the same RTT Pairing number as that of the 3′ RTT. The editing template of the 5′ PEgRNA and the 3′ PEgRNA can be completely complementary to each other, wherein the 5′ editing template and the 3′ editing template have the same length and perfect complementarity to each other. Alternatively, the 5′ editing template can comprise a region of complementarity to the 3′ editing template, and further comprise a non-complementary region to the 3′ editing template. The 3′ editing template can comprise a region of complementarity to the 5′ editing template, and further comprise a non-complementary region to the 5′ editing template. The non-complementary region can be at the 5′ end or the 3′ end of the editing templates. Contacting the target DMPK gene with the prime editing system can result in deletion of the sequence between the 5′ PEgRNA nick and the 3′ PEgRNA nick (the IND) and insertion of the region of complementarity between the 5′ editing template and the 3′ editing template (the OD), or insertion of the region of complementarity between the 5′ editing template and the 3′ editing template as well as the non-complementarity regions (the RD), and hence replace the CTG expansion with the sequence of the OD or the RD. Each of the 3′ PEgRNA is assigned a RTT pairing number, and can be used in a dual prime editing composition with a 5′ PEgRNA comprising an RTT having the same RTT pairing number.

In some embodiments, a prime editing composition comprises a 3′ PEgRNA or component thereof exemplified in Tables 34A and 34B and a 5′ PEgRNA, wherein the editing template of the 3′ PEgRNA comprises a region of identity to the spacer of the 5′ PEgRNA. In some embodiments, the editing template of the 5′ PEgRNA comprises a region of identity to the spacer of the 3′ PEgRNA. In some embodiments, the 3′ editing template comprises a region of complementarity to a region on the first strand of the DMPK gene that is directly 3′ to nucleotide 3 of the search target sequence of the 5′ PEgRNA. In some embodiments, the 5′ editing template comprises a region of complementarity to a region on the second strand of the DMPK gene that is directly 3′ to nucleotide 3 of the search target sequence of the 3′ PEgRNA. For example, the extension arm of the 3′ PEgRNA can comprise an editing template comprising the sequence of any of the RTT sequences provided in Table 40. Each of the RTT sequences in Table 40 is assigned a pairing 5′ spacer number. The 3′ PEgRNA can be used in a dual prime editing system with a 5′ PEgRNA, wherein the 5′ PEgRNA comprises a spacer that has the Pairing 5′ spacer number assigned to the 3′ RTT in Table 40. Contacting the target DMPK gene with the prime editing system can result in deletion of the sequence between the 5′ PEgRNA nick and the 3′ PEgRNA nick (the IND) without insertion of any exogenous sequence (a “clean deletion”). Alternatively, the 5′ editing template and the 3′ editing template can further comprise a region of complementarity to each other, and the region of complementarity can replace the IND in the DMPK gene. For example, in some embodiments, the 3′ PEgRNA is part of a dual prime editing composition that further comprises a 5′ PEgRNA, wherein the 3′ PEgRNA comprises a RTT comprising a sequence selected from sequence numbers 3477-3479, and the 5′ PEgRNA comprises a spacer comprising nucleotides 5-20 of sequence number 484. In some embodiments, the 3′ PEgRNA is part of a dual prime editing composition that further comprises a 5′ PEgRNA, wherein the 3′ PEgRNA comprises a RTT comprising a sequence selected from sequence numbers 3480-3484, and the 5′ PEgRNA comprises a spacer comprising nucleotides 5-20 of sequence number 77. In some embodiments, the 3′ PEgRNA is part of a dual prime editing composition that further comprises a 5′ PEgRNA, wherein the 3′ PEgRNA comprises a RTT comprising a sequence selected from sequence numbers 3485-3489, and the 5′ PEgRNA comprises a spacer comprising nucleotides 5-20 of sequence number 590. In some embodiments, the 3′ PEgRNA is part of a dual prime editing composition that further comprises a 5′ PEgRNA, wherein the 3′ PEgRNA comprises a RTT comprising a sequence selected from sequence numbers 3490-3494, and the 5′ PEgRNA comprises a spacer comprising nucleotides 5-20 of sequence number 1019. In some embodiments, the 3′ PEgRNA is part of a dual prime editing composition that further comprises a 5′ PEgRNA, wherein the 3′ PEgRNA comprises a RTT comprising nucleotides x-40 of sequence number 3664, 3665, 3666, 3667, 3668, 3669, 3670, 3671, 3672, 3673, 3674, 3675, 3676, 3677, 3678, 3679, 3680, 3681, or 3682, wherein the 5′ PEgRNA comprises a spacer comprising nucleotides 5-20 of sequence number 518, 536, 554, 572, 590, 39, 912, 929, 947, 965, 983, 1001, 1019, 1341, 20, 77, 484, 58, or 1 respectively in the same order, and wherein x is any integer from 1 to 31. Each 3′ PEgRNA is assigned a RTT pairing number, which corresponds to a pairing 5′ spacer number in Table 40. The 3′ PEgRNA can be used in a dual prime editing system or composition with a 5′ PEgRNA comprising a spacer of the same pairing 5′ spacer number. Preferably, the 5′ PEgRNA comprises a “clean deletion” RTT, for example, an RTT selected from Table 39 that has a Pairing 3′ spacer number that is 15.

### Tables 35A and 35B

Tables 35A and 35B provide exemplary 3′ PEgRNAs and components that can be used with any prime editor containing a Cas9 protein capable of recognizing a NGG PAM sequence, wherein N is A, G, C, or T. The PEgRNAs exemplified in Tables 35A and 35B comprise: (a) a spacer comprising at its 3′ end a sequence corresponding to nucleotides 5-20 of sequence number 2353, (b) a gRNA core capable of binding to a Cas9 protein, and (c) an extension arm comprising (i) an editing template and (ii) a primer binding site (PBS) comprising at its 5′ end the sequence corresponding to sequence number 2368.

The PEgRNA spacer can be, for example, 16-22 nucleotides in length. In some embodiments, the PEgNA spacer is 16-20 nucleotides in length. In some embodiments, the PEgRNA spacer comprises nucleotides 5-20, 4-20, 3-20, 2-20, or 1-20 of sequence number 2353. In some embodiments, the PEgRNA spacer comprises the sequence of sequence number 2353. In some embodiments, the PEgRNA spacer is 20 nucleotides in length. Any of these spacers exemplified can be referred to as 3′ spacer No. 16.

The PBS can be, for example, 3 to 19 nucleotides in length. In some embodiments, the PBS is 3 to 17 nucleotides in length and corresponds to a PBS sequence in Table 20A. In some cases, a PBS length of no more than 3 nucleotides less than the PEgRNA spacer length is chosen.

The 3′ PEgRNAs of Tables 35A and 35B can be used in a dual prime editing system or composition further comprising any suitable 5′ PEgRNA. The prime editing system can be used, e.g., for editing a DMPK gene harboring 50 or more CTG repeats in the DMPK gene.

In some embodiments, a prime editing composition comprises a 3′ PEgRNA or component thereof exemplified in Tables 35A and 35B and a 5′ PEgRNA, wherein the editing template of the 3′ PEgRNA and the 5′ PEgRNA each comprises a region of complementarity to each other. For example, the editing template of the 3′ PEgRNA can comprise any of the RTT sequences provided in Table 38. The terms editing template and RTT may be used interchangeably. Each of the RTT sequences in Table 38 is assigned a RTT pairing number that reflects the complementarity relationship between a 5′ RTT and a 3′ RTT. The 3′ PEgRNA can be used in a dual prime editing system that further comprises a 5′ PEgRNA, e.g., a 5′ PEgRNA having a spacer sequence and a corresponding PBS as exemplified in Tables 1A-19B, wherein the 5′ PEgRNA comprises an editing template comprising a RTT sequence having a region of complementarity to the 3′ RTT. The 5′ RTT can comprise any of the RTT sequences provided in Table 37, wherein the 5′ RTT sequence has the same RTT Pairing number as that of the 3′ RTT. The editing template of the 5′ PEgRNA and the 3′ PEgRNA can be completely complementary to each other, wherein the 5′ editing template and the 3′ editing template have the same length and perfect complementarity to each other. Alternatively, the 5′ editing template can comprise a region of complementarity to the 3′ editing template, and further comprise a non-complementary region to the 3′ editing template. The 3′ editing template can comprise a region of complementarity to the 5′ editing template, and further comprise a non-complementary region to the 5′ editing template. The non-complementary region can be at the 5′ end or the 3′ end of the editing templates. Contacting the target DMPK gene with the prime editing system can result in deletion of the sequence between the 5′ PEgRNA nick and the 3′ PEgRNA nick (the IND) and insertion of the region of complementarity between the 5′ editing template and the 3′ editing template (the OD), or insertion of the region of complementarity between the 5′ editing template and the 3′ editing template as well as the non-complementarity regions (the RD), and hence replace the CTG expansion with the sequence of the OD or the RD. Each of the 3′ PEgRNA is assigned a RTT pairing number, and can be used in a dual prime editing composition with a 5′ PEgRNA comprising an RTT having the same RTT pairing number.

In some embodiments, a prime editing composition comprises a 3′ PEgRNA or component thereof exemplified in Tables 35A and 35B and a 5′ PEgRNA, wherein the editing template of the 3′ PEgRNA comprises a region of identity to the spacer of the 5′ PEgRNA. In some embodiments, the editing template of the 5′ PEgRNA comprises a region of identity to the spacer of the 3′ PEgRNA. In some embodiments, the 3′ editing template comprises a region of complementarity to a region on the first strand of the DMPK gene that is directly 3′ to nucleotide 3 of the search target sequence of the 5′ PEgRNA. In some embodiments, the 5′ editing template comprises a region of complementarity to a region on the second strand of the DMPK gene that is directly 3′ to nucleotide 3 of the search target sequence of the 3′ PEgRNA. For example, the extension arm of the 3′ PEgRNA can comprise an editing template comprising the sequence of any of the RTT sequences provided in Table 40. Each of the RTT sequences in Table 40 is assigned a pairing 5′ spacer number. The 3′ PEgRNA can be used in a dual prime editing system with a 5′ PEgRNA, wherein the 5′ PEgRNA comprises a spacer that has the Pairing 5′ spacer number assigned to the 3′ RTT in Table 40. Contacting the target DMPK gene with the prime editing system can result in deletion of the sequence between the 5′ PEgRNA nick and the 3′ PEgRNA nick (the IND) without insertion of any exogenous sequence (a “clean deletion”). Alternatively, the 5′ editing template and the 3′ editing template can further comprise a region of complementarity to each other, and the region of complementarity can replace the IND in the DMPK gene. For example, in some embodiments, the 3′ PEgRNA is part of a dual prime editing composition that further comprises a 5′ PEgRNA, wherein the 3′ PEgRNA comprises a RTT comprising a sequence selected from sequence numbers 3477-3479, and the 5′ PEgRNA comprises a spacer comprising nucleotides 5-20 of sequence number 484. In some embodiments, the 3′ PEgRNA is part of a dual prime editing composition that further comprises a 5′ PEgRNA, wherein the 3′ PEgRNA comprises a RTT comprising a sequence selected from sequence numbers 3480-3484, and the 5′ PEgRNA comprises a spacer comprising nucleotides 5-20 of sequence number 77. In some embodiments, the 3′ PEgRNA is part of a dual prime editing composition that further comprises a 5′ PEgRNA, wherein the 3′ PEgRNA comprises a RTT comprising a sequence selected from sequence numbers 3485-3489, and the 5′ PEgRNA comprises a spacer comprising nucleotides 5-20 of sequence number 590. In some embodiments, the 3′ PEgRNA is part of a dual prime editing composition that further comprises a 5′ PEgRNA, wherein the 3′ PEgRNA comprises a RTT comprising a sequence selected from sequence numbers 3490-3494, and the 5′ PEgRNA comprises a spacer comprising nucleotides 5-20 of sequence number 1019. In some embodiments, the 3′ PEgRNA is part of a dual prime editing composition that further comprises a 5′ PEgRNA, wherein the 3′ PEgRNA comprises a RTT comprising nucleotides x-40 of sequence number 3664, 3665, 3666, 3667, 3668, 3669, 3670, 3671, 3672, 3673, 3674, 3675, 3676, 3677, 3678, 3679, 3680, 3681, or 3682, wherein the 5′ PEgRNA comprises a spacer comprising nucleotides 5-20 of sequence number 518, 536, 554, 572, 590, 39, 912, 929, 947, 965, 983, 1001, 1019, 1341, 20, 77, 484, 58, or 1 respectively in the same order, and wherein x is any integer from 1 to 31. Each 3′ PEgRNA is assigned a RTT pairing number, which corresponds to a pairing 5′ spacer number in Table 40. The 3′ PEgRNA can be used in a dual prime editing system or composition with a 5′ PEgRNA comprising a spacer of the same pairing 5′ spacer number. Preferably, the 5′ PEgRNA comprises a “clean deletion” RTT, for example, an RTT selected from Table 39 that has a Pairing 3′ spacer number that is 16.

Sequences of exemplary editing templates (or RTTs) are provided in Tables 37-42. Although all the sequences provided in Tables 37-42 are RNA sequences, “T” is used instead of a “U” in the sequences for consistency with the ST.26 standard used in the accompanying sequence listing.

Tables 37 and 38 provide exemplary 5′ RTTs and 3′ RTTs, respectively, wherein the 5′ RTT and the 3′ RTT each comprises a region of complementarity to each other. Tables 37 and 38 each includes five columns: from left to right, the first column provides the actual sequence of the RTT, the second column provides the SEQ ID NO., the third column provides the RTT length (in these RTTs, also the complementarity region length between two pairing PEgRNAs), the fourth column provides the GC content of the RTT, and the fifth column provides a RTT Paring number. For each 5′ RTT in Table 37 having a specific RTT Paring number, a 5′ PEgRNA comprising the 5′ RTT can be used in a dual prime editing system further comprising a 3′ PEgRNA, wherein the 3′ PEgRNA has the same RTT Paring number as assigned in Table 38. Although the pairing 5′ RTT and 3′ RTT sequences exemplified in Tables 37 and 38 have the same length and are perfectly complementary to each other, a skilled person understands that a 5′ RTT and/or a 3′ RTT can comprise additional nucleotides that are not complementary to the opposite side RTT.

TABLE 37
Exemplary 5′ RTT sequences

| | | | | RTT |
|---|---|---|---|---|
| | SEQ ID | RTT | RTT GC | Pairing |
| Sequence | NO: | length | content | NO: |
| GGCTTGTCGACGACGGCGGTCTCCGTCGTCAGGATCAT | 2691 | 38 | 0.63 | 1 |
| TTTCCAAGATTTGCAACCGGCAACTGTATGCTATTACC | 2692 | 38 | 0.421052632 | 2 |
| TATTGTTCCGGTTTATCACGAAGCGTACTCGGATTACA | 2693 | 38 | 0.421052632 | 3 |
| TATCGCACCACCCTATGCGTTCCATATCAATACTTATC | 2694 | 38 | 0.421052632 | 4 |
| TTCGTCTACAGTATCCTTCTTTCAACCTATTCACCGCA | 2695 | 38 | 0.421052632 | 5 |
| TGTTATGGTGTGAATACTTCATGGCGGATGATTTCCAC | 2696 | 38 | 0.421052632 | 6 |
| TATGTATACGAAGCCCATGCAAACATATTGCCGATCCA | 2697 | 38 | 0.421052632 | 7 |
| TTACCGTGTCTACATGGTATTTGGCATTTGCGATTACC | 2698 | 38 | 0.421052632 | 8 |
| TATACAAGGTACATATACCAACGGGCACTCCAACAGAA | 2699 | 38 | 0.421052632 | 9 |
| TTACTGCGTATACTTGGAAATTTACACGTCCAGAGGGA | 2700 | 38 | 0.421052632 | 10 |
| TGTATGTACAGGGTATACGGTCAGGATATGGCACCGCC | 2701 | 38 | 0.526315789 | 11 |
| TCACCCTCGACAAGTACAGGTACTCGAATGCACGGACA | 2702 | 38 | 0.526315789 | 12 |
| TTCCGAAAGCTTTCGCTCTCTCAGGCGTATTCTGGAGC | 2703 | 38 | 0.526315789 | 13 |
| TGGCGGAGAAATCAATTGCTCTCAACCCGTGTTGCTCC | 2704 | 38 | 0.526315789 | 14 |
| TCACGTTCCTCATCTTGTTACCACGTGCAGGCCCATAC | 2705 | 38 | 0.526315789 | 15 |
| TCTCGTCAAGGGAACCAAACACGAGTATGTGTGGGCTC | 2706 | 38 | 0.526315789 | 16 |
| TATACTGTGCGCCGACATACGAGGGTTTCCTGTCACTC | 2707 | 38 | 0.526315789 | 17 |
| TCTCTGGAATCTTTCGTTTCTCACGGGTGTGGTGTGGC | 2708 | 38 | 0.526315789 | 18 |
| TACCTATGTACACCAGAACCAGTCCTCGTGTGCTCCAC | 2709 | 38 | 0.526315789 | 19 |
| TACCGGAGGGCGACACTCGAGGTTGTGACCTCCCACAC | 2710 | 38 | 0.631578947 | 20 |
| TCGTCCCTGTCCGCGGCCGAGAGTATTGTACGCCCACA | 2711 | 38 | 0.631578947 | 21 |
| TCACGATCGATCGCGCGGAACTGGGTATGCTCCACCGC | 2712 | 38 | 0.631578947 | 22 |
| TTCTCGGAGGCATGCGGGCATTTGCATGGCTCCGTCCC | 2713 | 38 | 0.631578947 | 23 |
| TCTCTGCCGCTATGCGTTCACGCCATGGCAAAGGCGCC | 2714 | 38 | 0.631578947 | 24 |
| TGCATGATGGTCCGATGCTGCCTGCCTCGGCCTCATCC | 2715 | 38 | 0.631578947 | 25 |
| TGCGTCGACGCTTGCGCATACATGCAGAACGGGCGGTC | 2716 | 38 | 0.631578947 | 26 |
| TCAGCTCTGTCGAGGCCCAAGCCGTGGTCCTTGGTGAC | 2717 | 38 | 0.631578947 | 27 |
| TCTTCCGGTACGCGTCTCCTGGGTTTCGCCCGAACCGA | 2718 | 38 | 0.631578947 | 28 |
| TGGCGCACCGCGGCGGTATCTCGGGCCGAGCTCTGTAC | 2719 | 38 | 0.710526316 | 29 |
| TGCCAATTGAGCCGAACAGCGCCTGCGGGCCTGCCGGC | 2720 | 38 | 0.710526316 | 30 |
| TGTCACACATGCTCCCGGCACCTCGCCCGACGCACGGC | 2721 | 38 | 0.710526316 | 31 |
| TGCGGGCGAACGCTGCTTTCAGCGGCACCGGGCACCTC | 2722 | 38 | 0.710526316 | 32 |
| TTGCCGCACGCCGAGCCTTCTCTACGGGCCCGGGCGAA | 2723 | 38 | 0.710526316 | 33 |
| TGCCCGCCACGCTGCGTCCAGGGACGGGAAACGTCACC | 2724 | 38 | 0.710526316 | 34 |
| TACGGCGGCCGGACCTCTCCGCGTTACGCCTGCATGGC | 2725 | 38 | 0.710526316 | 35 |
| TGCTGCGAGAACCGCCTATCAGCCGGGCGCACGCTGCC | 2726 | 38 | 0.710526316 | 36 |
| TGTGCCCAGGCTTTATCGGCCGCCTGCCGCGCGTTGCC | 2727 | 38 | 0.710526316 | 37 |
| TGCATCGCGGTGCGGCCCGAGCCGTCGGCGGGCCTTCC | 2728 | 38 | 0.789473684 | 38 |
| TACGCACACCCGGCCCGGGAGCGACTCGGCCGCGTGGC | 2729 | 38 | 0.789473684 | 39 |
| TGGCCGACTCCGGACCGTCACCTCGCGCGGAGCGCGGC | 2730 | 38 | 0.789473684 | 40 |
| TGCGCGCCACCACGCGCGCGTGGCCTCCGCACGAGACC | 2731 | 38 | 0.789473684 | 41 |
| TGGCGGATGAGCCGCCCAGAAGGCGGCGAGGGCCGGCC | 2732 | 38 | 0.789473684 | 42 |
| TGCCAGGTCGACCGAGGCCGCGGGCCCGGGAAGACGGC | 2733 | 38 | 0.789473684 | 43 |
| TCGCCCGGCGCCGGGATGTGCGCCGCGGAGCCCGGAAA | 2734 | 38 | 0.789473684 | 44 |
| TCGAAGCCGCGGCTGGCGCGGACGTGCCCGGCGAGCAC | 2735 | 38 | 0.789473684 | 45 |
| TCCGTACGGGCCCAGGGTACGCCCGCGTGCCGGGCAGC | 2736 | 38 | 0.789473684 | 46 |
| TCCGCCTCTTATCTGTGGGCCGC | 2737 | 23 | 0.652173913 | 47 |
| TGCCAACCGTCGGGTCCATGCGA | 2738 | 23 | 0.652173913 | 48 |
| TTCGAGCGCACCCGCAACAAGCC | 2739 | 23 | 0.652173913 | 49 |
| TTCGCGCAGCAGGGTTGCGAACC | 2740 | 23 | 0.652173913 | 50 |
| TTTGGCCGGTGCGGGACAGGTAC | 2741 | 23 | 0.652173913 | 51 |
| TCGTTTGGCGATGGCCTCCTCCC | 2742 | 23 | 0.652173913 | 52 |
| TCTCGGCGAGCCGTCTACGTGTC | 2743 | 23 | 0.652173913 | 53 |
| TCGTGGTCTCGGGCATCAGACCC | 2744 | 23 | 0.652173913 | 54 |
| TGCCCAGTCCGTGTCTCTGCGCA | 2745 | 23 | 0.652173913 | 55 |
| TGCGTTTGTGCAATGGGCGGCGCAGTGGGCCGCGCATGTATCGCCA | 2746 | 53 | 0.622641509 | 56 |
| CAGATCA | | | | |
| TCGGAGGCCACGGTCGCCACAGCAACGTGGCTGTTTCACTATGGAT | 2747 | 53 | 0.622641509 | 57 |
| GTCGGGC | | | | |
| TCTCGGTCACGCACGGCGCAGCTCGGACAACCCGACACGCATTGCT | 2748 | 53 | 0.622641509 | 58 |
| ACTCCAA | | | | |
| TTGCTCCGTGGTATCGGTCCCGAGCCATATCAGGTTTGCCGCCGCGC | 2749 | 53 | 0.622641509 | 59 |
| AAGGGA | | | | |
| TTGGGTGTGGCGCCAACGGGAGTGCGGCCTGGGTACCTATTATTGC | 2750 | 68 | 0.632352941 | 60 |
| GGCCGTTGTGGTGGAGAGGACC | | | | |
| TCGCTGGCGTCGTACGCGGAGTGTGCAAGAGCTGGGTGGCGAAAGT | 2751 | 68 | 0.632352941 | 61 |
| CAAGCAGACCCTGGCTGCACCA | | | | |
| TCGGCTGTGGCAGGCCGGGAAGCTCCATGTACACGCTGCAAGGCGA | 2752 | 68 | 0.632352941 | 62 |
| AGCTACCCAGCCGTCAGGAACA | | | | |
| TTACCGGGCAGTGTCCTTTCTGCGCAGCAGGGTCGTACCGGACTACG | 2753 | 68 | 0.632352941 | 63 |
| CAGGAGGAAGGCTTCCGCGTC | | | | |
| TACAACGCGGATCAGGGCATCCATCCACAGCGTGCGCGAACGGCGG | 2754 | 68 | 0.632352941 | 64 |
| TGTCCCGAGATGGGTGGTATGC | | | | |
| TCCTCTCGCCTGAATCGGACTGGACAGCTTCGTGGGATGGCTCGAAG | 2755 | 68 | 0.632352941 | 65 |
| AGCCTCCGGAGGCGGTCTACC | | | | |
| TTGCCGACTGATACTGCGTCCTTACGCCCGATTGCGTTACAAGGGTG | 2756 | 68 | 0.632352941 | 66 |
| CAAGCGGACGCCCGCGCAGCC | | | | |
| TCCCGGAGCGAAACCGGCCAACGAAGCGAAAGGGTGGCCCTGCAGT | 2757 | 68 | 0.632352941 | 67 |
| GGATATGTGGCGGCTGTCTTCC | | | | |
| TCCAGCAAGAGGCAACCCACCCACGCGAAGCCACCACTTGCGCCGA | 2758 | 68 | 0.632352941 | 68 |
| CGACGGAACCTCGCTCGATTTC | | | | |
| TCCGAGGAGGACAGAGCCAAGGACAGCCCTTCCTCAATCTACTCAC | 2759 | 83 | 0.626506024 | 69 |
| GATCGACGTGCGCGGCCTTGAAGCGGCGAGGTCACGC | | | | |
| TCCCGCCTTCACACCCTCGAGATACTGCGCGCTCAGCCTTCACACCT | 2760 | 83 | 0.626506024 | 70 |
| ATCGCGCACAGTCGGACCGGGCTGTACTGGATGGGA | | | | |
| TTGCAGCCAATTGTGGCAACACCGGAAGCCAAGGCACAGGATGCCG | 2761 | 83 | 0.626506024 | 71 |
| ATCGCCGGTGGGCGCGCCACTTGCTGGCCAAGCTATC | | | | |
| TTGGCCAAAGACACCACGAGAAATGCCCAATCAGGGTGGCAGGGAC | 2762 | 83 | 0.626506024 | 72 |
| TGCCACGAAGCGGACTGCGACGAAGGCGCGCACAGCC | | | | |
| TTACTGAGTGGCCCGTACGCGCAGGGCCGTCGGCAGAACTCAGTGT | 2763 | 83 | 0.626506024 | 73 |
| CGACCGACGGCAGATGGTCCGTCTTCTGGTCATCCCA | | | | |
| TCGCGGCCTGCGGTTCGGTACGTTGCGTTTCAAACCGGTCTTCCTGC | 2764 | 83 | 0.626506024 | 74 |
| ACGTCTGCTTGCGGAGCTTCGAAAGTCGCCGGAGCC | | | | |
| TGGCGCCTGAGCCGTTTGAAAGAGGCACTCGTGCCATATTGCCTCGA | 2765 | 83 | 0.626506024 | 75 |
| CGCCTCTGGCAACGCCACTCCGGTGTCCCGTGTGGA | | | | |
| TCATACGCGGCAATCGCGTCCTTCTGTACGGACCCACTACGGTTGCC | 2766 | 83 | 0.626506024 | 76 |
| GCTATGTCAGAGACGGCGGCGTTGCACAGCCGTGCC | | | | |
| TCCCACTACCGTCAGACTCGCTCAGAACAGACCAGATCACGGCGGC | 2767 | 83 | 0.626506024 | 77 |
| GGGATCCGCCTTGTCAAGGAGAGCGGAGCGTACACCC | | | | |
| TGGGCGACTTACGATTGGAGCATTCTCCTCGGCTTTGTTGGCTCAGC | 2768 | 83 | 0.626506024 | 78 |
| GCGACCCGCTGGGAGGAGGAGGACACCTGGGAGCGA | | | | |
| ACGATGATGGTGATGATGGTGTGC | 2769 | 24 | 0.5 | 79 |
| TAAGTCAAATGAGACTTT | 2770 | 18 | 0.28 | 80 |
| TTAAGTGAGCTGCTT | 2771 | 15 | 0.4 | 81 |
| CAGACGGTGGCGCACTCATGCCACGCTT | 2772 | 28 | 0.64 | 82 |
| TCTTGTGAATGGAATTGAGGAGCTT | 2773 | 25 | 0.4 | 83 |
| GGAGAGAGGCGGGCTCTCTTTCCTT | 2774 | 25 | 0.6 | 84 |
| CGGGCCACCGCCCTT | 2775 | 15 | 0.8 | 85 |
| GCAAGGTGTTTGTGCACCTTACCTT | 2776 | 25 | 0.48 | 86 |
| TTTACACTAGATGGTGCTGT | 2777 | 20 | 0.4 | 87 |
| TCTGCACAAGAGATATTCTGTTCTCCATGT | 2778 | 30 | 0.4 | 88 |
| TAAAGGATCACATATAGGTCGTGGT | 2779 | 25 | 0.4 | 89 |
| GCCGGCGGAAGCCGGCTGGT | 2780 | 20 | 0.8 | 90 |
| TCTGCCCTCCTCTTGGGT | 2781 | 18 | 0.61 | 91 |
| AAAGGGTTGCACTTTGCTGATACGTGGGGT | 2782 | 30 | 0.5 | 92 |
| GCCGGGGTATGGGCACACGCCCCGCGGGGT | 2783 | 30 | 0.8 | 93 |
| GTGATCCAAGTGCTCGACACTCCCACGGGT | 2784 | 30 | 0.6 | 94 |
| GGGTGCGGCGCAAGGGCCGCCTCGT | 2785 | 25 | 0.8 | 95 |
| TTGCCCTATCGGCGT | 2786 | 15 | 0.6 | 96 |
| CCCCTGCGCGGACCGCGCCTTCCGT | 2787 | 25 | 0.8 | 97 |
| GCGCCGTTGCGGCCGCGCCCGT | 2788 | 22 | 0.86 | 98 |
| TGTGGGACGTTACATTGAGTATTGAGCGCGCACGT | 2789 | 35 | 0.51 | 99 |
| ATGGGGCCTTAACGACCCGCGCCCCGCCTCTCAGT | 2790 | 35 | 0.69 | 100 |
| GGAGGGGAGTACGCAGCGCCCCAGT | 2791 | 25 | 0.72 | 101 |
| ACCTTTGGCCGTTCT | 2792 | 15 | 0.53 | 102 |
| CATAGCTGCT | 2793 | 10 | 0.5 | 103 |
| CACTATTACAGGAAATTGAGTAACTGGGCT | 2794 | 30 | 0.4 | 104 |
| GCCGGGGCATGGGCT | 2795 | 15 | 0.8 | 105 |
| TGCCGCACGGCGGCT | 2796 | 15 | 0.8 | 106 |
| GTTACACAAGTAGCT | 2797 | 15 | 0.4 | 107 |
| GGGCCTAGCT | 2798 | 10 | 0.7 | 108 |
| AGCTCAGATTATAGTGAGCT | 2799 | 20 | 0.4 | 109 |
| GTCGCGCTGTTCCGCGAGCT | 2800 | 20 | 0.7 | 110 |
| TAGATCAATTTCCGAGATCACTGTCATCTCAAGCT | 2801 | 35 | 0.4 | 111 |
| ACGACCGAGGGTCCT | 2802 | 15 | 0.67 | 112 |
| AATTTCAAGATCCTGCCT | 2803 | 18 | 0.39 | 113 |
| GGCCTGGACGCCAGGCCT | 2804 | 18 | 0.78 | 114 |
| GTCACCTACGCGCCT | 2805 | 15 | 0.67 | 115 |
| TCGGCCGGTCTTCCGCCT | 2806 | 18 | 0.72 | 116 |
| CCGCCGGCTCATCGAACCGCCT | 2807 | 22 | 0.73 | 117 |
| GCGAACCGACGTCACGGCTCGGGGTACTGAGGACT | 2808 | 35 | 0.66 | 118 |
| GGTACTACGTAAGCAGATAAGGTAAACACT | 2809 | 30 | 0.4 | 119 |
| ATACTGCCGGCCGAGAGTAAAGGACAGGTCATTAGGGTAT | 2810 | 40 | 0.5 | 120 |
| CTTGGACGGAAATCGCCTAT | 2811 | 20 | 0.5 | 121 |
| TTGAGCGATGCATAT | 2812 | 15 | 0.4 | 122 |
| GGTCCAGGCAAGACGCTGAT | 2813 | 20 | 0.6 | 123 |
| CGAGCTTACCCGTGGGGAAGCAAGGGAT | 2814 | 28 | 0.61 | 124 |
| GGACCAGTGTCCGTGGCGAGAT | 2815 | 22 | 0.64 | 125 |
| AGTAACTACGCAGAT | 2816 | 15 | 0.4 | 126 |
| GTTTCACATGGCCTGGACTCAT | 2817 | 22 | 0.5 | 127 |
| CTAGCGTGGCAGGTCTGCAT | 2818 | 20 | 0.6 | 128 |
| TTGCCCCATTGGCAT | 2819 | 15 | 0.53 | 129 |
| TGTGCCCGAGGGAGAATCGAACACATGAAT | 2820 | 30 | 0.5 | 130 |
| GCTACGGGTTCGCCTGAGCAAT | 2821 | 22 | 0.59 | 131 |
| AGGATCATCGATTTG | 2822 | 15 | 0.4 | 132 |
| ACAGCTGGGCAGGTG | 2823 | 15 | 0.67 | 133 |
| CATCGGTGGTCACTTGGCGGAGCTCGTG | 2824 | 28 | 0.64 | 134 |
| AGAGCGGCGGCCCGGCCGTG | 2825 | 20 | 0.85 | 135 |
| AATCGGCGAACGAAACCCGAAGCGCACGTG | 2826 | 30 | 0.6 | 136 |
| GCCCTGTCCGTAGTG | 2827 | 15 | 0.67 | 137 |
| CATTGCATACCTATAGTG | 2828 | 18 | 0.39 | 138 |
| GGCCGCAGACCGAAGCGGTGCAGTG | 2829 | 25 | 0.72 | 139 |
| GCGGCCGTCCCGGGGCTG | 2830 | 18 | 0.89 | 140 |
| GCCTAGGCTG | 2831 | 10 | 0.7 | 141 |
| CCCCAACCGCCGGCGGCCGATG | 2832 | 22 | 0.82 | 142 |
| AGCCTGCCCCGGGCCGCGGTGG | 2833 | 22 | 0.86 | 143 |
| TGAGCAAGATTCTCTCTAATTTCTTCCTGG | 2834 | 30 | 0.4 | 144 |
| CTGACACTGG | 2835 | 10 | 0.6 | 145 |
| AAGACACCGCCTGGGCCCCGATTGATATGG | 2836 | 30 | 0.6 | 146 |
| GGCCGGACGGGGCGGCGGGG | 2837 | 20 | 0.95 | 147 |
| CACCCGGCCCGCCGCTCCCGGGCCCGGGGCCGGGG | 2838 | 35 | 0.94 | 148 |
| CACCGGCGGG | 2839 | 10 | 0.9 | 149 |
| TGGTACGGCCGGATTCCGGG | 2840 | 20 | 0.7 | 150 |
| CCGGGTGGCGCCGGG | 2841 | 15 | 0.93 | 151 |
| TCGGCGGCCGGGCTCAGGCGCCGGG | 2842 | 25 | 0.88 | 152 |
| GGCGAGGCAGCCCCAAGGCCCCGGG | 2843 | 25 | 0.84 | 153 |
| GTTCGCGGGTGGGCGTTACGGG | 2844 | 22 | 0.73 | 154 |
| CTACCCTCCTCCGCGACGGG | 2845 | 20 | 0.75 | 155 |
| GTGAAGTCGGCGTGACCGAGGGCACGGG | 2846 | 28 | 0.71 | 156 |
| CCGGCAAGGG | 2847 | 10 | 0.8 | 157 |
| TGGTAGTTTCCGGTTTCGGCCTCGG | 2848 | 25 | 0.6 | 158 |
| TCGGACACCCTAGACCTCGG | 2849 | 20 | 0.65 | 159 |
| GGCGCGGGCGGGCCGGGATCGG | 2850 | 22 | 0.91 | 160 |
| GCCCCACGGGCCGAGTGCGG | 2851 | 20 | 0.85 | 161 |
| AGCTGCAGCCCGCCTCCACGACGGGCGG | 2852 | 28 | 0.79 | 162 |
| CCCGCGCCGCCAGCCCTAGGGTGGCCGCTCGGCGG | 2853 | 35 | 0.86 | 163 |
| GCGGGGCCCGGCGAGTTTGGCAGCGCGACGAGCCTCGCGG | 2854 | 40 | 0.8 | 164 |
| GCCGCCCGCCTCCGCGGCGCGG | 2855 | 22 | 0.95 | 165 |
| GCCGTAACCTCCGCGCGG | 2856 | 18 | 0.78 | 166 |
| GCGCCGGCGCGCCAGGCGCGGCCCGGCCGTGCCCGCGCGG | 2857 | 40 | 0.95 | 167 |
| CGCTGGGGCCTCCCGTCGGGGCCTGGCACGCGCGG | 2858 | 35 | 0.86 | 168 |
| GCCAGCGCGG | 2859 | 10 | 0.9 | 169 |
| ATTGTTCGCAAGCGG | 2860 | 15 | 0.53 | 170 |
| GGGGCCCGGCCTCAGGGGCCGG | 2861 | 22 | 0.91 | 171 |
| CCTTTAGAAGGCCAGGCCGG | 2862 | 20 | 0.65 | 172 |
| GCCCCGCAGCGGCACGGCCTGTGGCGCCGG | 2863 | 30 | 0.87 | 173 |
| TGTCCGCCGG | 2864 | 10 | 0.8 | 174 |
| CTAGCTTCGATCGCTACGGAGCCGG | 2865 | 25 | 0.64 | 175 |
| TCGACGGTATCGGCCCAGCCTGCGAGCCGG | 2866 | 30 | 0.73 | 176 |
| ACGGCGCGCAACAACGGCCACCTCCCGG | 2867 | 28 | 0.75 | 177 |
| GTACGAGCCGGGCCGCCGGGGAAGCCGTTCCCCGG | 2868 | 35 | 0.8 | 178 |
| CGGACGTGCTACCGG | 2869 | 15 | 0.73 | 179 |
| ATGGCAGTAGGTGTCAATACTACGG | 2870 | 25 | 0.48 | 180 |
| CGGAGGACGG | 2871 | 10 | 0.8 | 181 |
| TGCGGGCGGCCGTGTATAGG | 2872 | 20 | 0.7 | 182 |
| TGCGGGTCACCGAGG | 2873 | 15 | 0.73 | 183 |
| GCCACGAGGCTCAGG | 2874 | 15 | 0.73 | 184 |
| TTAAAGGGGACAATAACGCTTAAGG | 2875 | 25 | 0.4 | 185 |
| GAAACTTAAAGATGATAAGG | 2876 | 20 | 0.3 | 186 |
| GGAGACCGAGCGTGTGCGACGCTCCTGTCG | 2877 | 30 | 0.7 | 187 |
| CGCTCCGCGTCCCAATAGGTCG | 2878 | 22 | 0.68 | 188 |
| CCTTGTGGGCTGCGCCGAGCAGTCG | 2879 | 25 | 0.72 | 189 |
| GCCGCCCTGGCCTCG | 2880 | 15 | 0.87 | 190 |
| GGGCCAAGCCGCAGGTACCGGAGAACCCGGTGGCG | 2881 | 35 | 0.74 | 191 |
| AGCATATTTCACCTATTGGACCTGGGCG | 2882 | 28 | 0.5 | 192 |
| GCGCGCGGGCATTGGGCGCGGGCCGGCGCGGGCTGGGGCG | 2883 | 40 | 0.9 | 193 |
| CCGAGCGGCCACGAGGATCGGGGCG | 2884 | 25 | 0.8 | 194 |
| GCCCGCTCAGCCTCCCCGGCCCGGGGCG | 2885 | 28 | 0.89 | 195 |
| CCGGGGACGCCACGCGCGGTGCGCGGCG | 2886 | 28 | 0.89 | 196 |
| TACCCGGCCGCTCCGGCG | 2887 | 18 | 0.83 | 197 |
| GCTGGGCGACGGCCGGCGCG | 2888 | 20 | 0.9 | 198 |
| CCTACCACCCTCCGCAGCGCGCGCG | 2889 | 25 | 0.8 | 199 |
| AAGTAGGGCAGGGGACATCCGGTCCCGCTAGCGCG | 2890 | 35 | 0.69 | 200 |
| CGCCGCCAGCGCGCGACGCGGTGCCGGCCGCCGCG | 2891 | 35 | 0.91 | 20 |
| GGGGCGGCGCCGGCGGCCGGCTCCCGCG | 2892 | 28 | 0.96 | 202 |
| GCGTCGCCCGCCGGCAGCGGACGCG | 2893 | 25 | 0.88 | 203 |
| CGAAGGAGGGGTTCCTCGCGGAGCG | 2894 | 25 | 0.72 | 204 |
| CGAGCGAGCG | 2895 | 10 | 0.8 | 205 |
| AATCTTGCATTTGTCTTAGCAAGATCTCCG | 2896 | 30 | 0.4 | 206 |
| CGTCGCCCGGACGGGGCCCGGCCCACGCGCGAGTCTGCCG | 2897 | 40 | 0.85 | 207 |
| ATCGCGCGCTGGTGGCCG | 2898 | 18 | 0.78 | 208 |
| CCCTCGTACAGCGCGCCG | 2899 | 18 | 0.78 | 209 |
| GCACGGGGCACGTCCCGGGACGTAGCCG | 2900 | 28 | 0.79 | 210 |
| CCCGGCGACCAGCCG | 2901 | 15 | 0.87 | 211 |
| GTGCGCCGGGCCCCGCGCCCCAGCCTCCCG | 2902 | 30 | 0.9 | 212 |
| CGCACGCGGGGCCCG | 2903 | 15 | 0.93 | 213 |
| TTGCGGTGGTGTCGGGTGCGGCCCG | 2904 | 25 | 0.76 | 214 |
| CATCAGCGGAGCTCCCCG | 2905 | 18 | 0.72 | 215 |
| GAGGGCGGTTAGAGTGCCATTACCG | 2906 | 25 | 0.6 | 216 |
| GAAAGTCGCTGCGCGCGGACCG | 2907 | 22 | 0.73 | 217 |
| CCCCGGGCCGGACCG | 2908 | 15 | 0.93 | 218 |
| CGATCTTCCCGAACACCG | 2909 | 18 | 0.61 | 219 |
| TGCACGGGTACACTGAAGCCCTACG | 2910 | 25 | 0.6 | 220 |
| ATATCTCGTTGGTATTTAATATGATACG | 2911 | 28 | 0.29 | 221 |
| GGGCGTCGGACCCATCGACG | 2912 | 20 | 0.75 | 222 |
| GACACGCGCGGCACG | 2913 | 15 | 0.8 | 223 |
| CTGAGGGGCCGTGTTGCGACCCACG | 2914 | 25 | 0.72 | 224 |
| TCCTAACAGCGGACGACCCACG | 2915 | 22 | 0.64 | 225 |
| TCCCCTCCCGGCACGCCGCGCGGCTCGCCAGAACG | 2916 | 35 | 0.8 | 226 |
| TTACCTATTACCAAGGGCTACATCTTAG | 2917 | 28 | 0.39 | 227 |
| TAGCACTCTATCGCATAAGTCATTCTAG | 2918 | 28 | 0.39 | 228 |
| GCAGTGCTAG | 2919 | 10 | 0.6 | 229 |
| GCGCGAACCTGGGATCGCCATGCATGATAG | 2920 | 30 | 0.6 | 230 |
| GCCGACTCAGTGCTGCTCTGAG | 2921 | 22 | 0.64 | 231 |
| GGAGAGATCACGCTCGATACGAGGGGAG | 2922 | 28 | 0.61 | 232 |
| ATGGCGGACTCAGCGCGCCGAAGAACGGAG | 2923 | 30 | 0.67 | 233 |
| ACTTCACATCCAGTGACGAG | 2924 | 20 | 0.5 | 234 |
| AAGCGAGTCAGAAGAGAGAG | 2925 | 20 | 0.5 | 235 |
| CAGCGCCCCGCCGGCGTACCGCCCTAGCAG | 2926 | 30 | 0.8 | 236 |
| GCCTTACTCGACCGCCACGACCTCGTCCAG | 2927 | 30 | 0.67 | 237 |
| CCTCGGATGGGGCCGCGGCTACGGGTACAG | 2928 | 30 | 0.73 | 238 |
| AATTCTGATGTAAAG | 2929 | 15 | 0.27 | 239 |
| TGATAGGTAAGGTGTAATTACTTTC | 2930 | 25 | 0.32 | 240 |
| GTACGGCTTC | 2931 | 10 | 0.6 | 241 |
| TGGGGTACTTATGTCGTC | 2932 | 18 | 0.5 | 242 |
| CTATCCGGTCCGAGCGTC | 2933 | 18 | 0.67 | 243 |
| CTGAGCGGCGAAGGCATCCCGGCCTCCGTC | 2934 | 30 | 0.73 | 244 |
| GCTGTGGGTCGCAGTCACGCGGGCCGTC | 2935 | 28 | 0.75 | 245 |
| GCACACAACGACGTC | 2936 | 15 | 0.6 | 246 |
| TAGGGAAGAGTCGAATATATCTCTC | 2937 | 25 | 0.4 | 247 |
| AACCCGCAGAGGGACGGGTCGTTCGCCCTC | 2938 | 30 | 0.7 | 248 |
| ACTACGGGTGGGAACCTC | 2939 | 18 | 0.61 | 249 |
| CTGTGCATCGCACTC | 2940 | 15 | 0.6 | 250 |
| TCAAACATGAATTCTTCACGTTGTTAAAGATTATC | 2941 | 35 | 0.29 | 251 |
| GGTCCATTGGCACTAGGGGCGTTCAGTATCATATC | 2942 | 35 | 0.51 | 252 |
| TAACTGCTCTATATACGAGATTATCAAATC | 2943 | 30 | 0.3 | 253 |
| CGATACGGCGCCCACGGCGGTGCATTGC | 2944 | 28 | 0.71 | 254 |
| TCCCTGTCCGCCACCTGGCGCTGCTGGCGTGGACATGTGC | 2945 | 40 | 0.7 | 255 |
| CCGGCGCAGGGCGGACCGACGAGAGCGTGC | 2946 | 30 | 0.8 | 256 |
| ACTCTATCGATTATCTGC | 2947 | 18 | 0.39 | 257 |
| ACTGCCGGGGCCGGCGCCGTCGCAGCGTCCGATGC | 2948 | 35 | 0.8 | 258 |
| GAAGAATAAACGTTGGATCTCACATCATGC | 2949 | 30 | 0.4 | 259 |
| AAACAGACATTGTGCCTGTGCATGC | 2950 | 25 | 0.48 | 260 |
| CGCGCTCCAGCGGTCGGGGCCTGGC | 2951 | 25 | 0.84 | 261 |
| TGGAAATGCCGTGGCGTGGGGC | 2952 | 22 | 0.68 | 262 |
| ATGCGCGCGCTGTCGGGC | 2953 | 18 | 0.78 | 263 |
| CGGGCGGCGGGAGCGGGC | 2954 | 18 | 0.94 | 264 |
| GGCAACGGGACGTCCCCGGGGCGGC | 2955 | 25 | 0.84 | 265 |
| CCAGTCGGCCCTGCTCCGGC | 2956 | 20 | 0.8 | 266 |
| ACGCAGGCCTTGCCTCCACGACGGC | 2957 | 25 | 0.72 | 267 |
| CGTGCCCGCAGCCGGTTGGAGTGTGAGCGGGAGGC | 2958 | 35 | 0.74 | 268 |
| GCGGTGGGCGGAGGC | 2959 | 15 | 0.87 | 269 |
| AGCGGCTTCGAATCTCAGGC | 2960 | 20 | 0.6 | 270 |
| CGACCGGTGCGGGCCCTCGTCGAGGAGCAAGGGTGCAGGC | 2961 | 40 | 0.75 | 271 |
| AGGAAACGGCGACCTGAGGTTTCGC | 2962 | 25 | 0.6 | 272 |
| TGGACCGCCACAGGCTTGGCCCTAGCTCGC | 2963 | 30 | 0.7 | 273 |
| GCCCCGGGCGACTGGCACTCGC | 2964 | 22 | 0.82 | 274 |
| CGAAGTGGTGTAGCTGACATCGCGC | 2965 | 25 | 0.6 | 275 |
| CCCGGGTGGTGGCCGCGCCGGTCAGCGCGC | 2966 | 30 | 0.87 | 276 |
| CGAGGTAGAGGCGAGGAGTAGACGCCGCGC | 2967 | 30 | 0.7 | 277 |
| GATCTAAGAACTGTCTCCGC | 2968 | 20 | 0.5 | 278 |
| TCGTGTTCAATCCGC | 2969 | 15 | 0.53 | 279 |
| GGACATTCTTGCCGC | 2970 | 15 | 0.6 | 280 |
| GGTCCTGGTGACGACGGGGTGAGAGCGAACTTAAGGCCGC | 2971 | 40 | 0.65 | 281 |
| AGCCCGCGCGTTACACGC | 2972 | 18 | 0.72 | 282 |
| GAACACTCCGGCCGTAGC | 2973 | 18 | 0.67 | 283 |
| CCGGTCGTCCGTTCAGAGACCATGCGGAGC | 2974 | 30 | 0.67 | 284 |
| GCGCCCAGAAGTGGGGAAGCCGCTGCCAAGAGAGC | 2975 | 35 | 0.69 | 285 |
| CTCGCTCTAAGATTTAGCCGCTGAAAGAGC | 2976 | 30 | 0.5 | 286 |
| ATGTACGGGCCGTCC | 2977 | 15 | 0.67 | 287 |
| ACGCCATGGAGGGATAGTCC | 2978 | 20 | 0.6 | 288 |
| GTCGCTGATTTCGGTGCGAGCCTCC | 2979 | 25 | 0.64 | 289 |
| CTGCCCCTGAGGGACGATCC | 2980 | 20 | 0.7 | 290 |
| GCACCGAAGCGCGGTCCAATGGACATCC | 2981 | 28 | 0.64 | 291 |
| TCCAAGGCCACCCCGCGGGTACCGGAATCC | 2982 | 30 | 0.7 | 292 |
| CGCGCACGCCGGGGTGCC | 2983 | 18 | 0.89 | 293 |
| CCGCGTGGGGTGGCGGTGCCCCGGTGCC | 2984 | 28 | 0.86 | 294 |
| CTCACGGGTCCCCGAGCCCGCTGCC | 2985 | 25 | 0.8 | 295 |
| CGCCCGGCCCGTTGAACGGGTCACTGGCATCGGCC | 2986 | 35 | 0.74 | 296 |
| GCGCACCGGAGCGGAGGCAGGGCCGGCC | 2987 | 28 | 0.86 | 297 |
| CCCGGGCGCCCCGCGAGGCCCGGCC | 2988 | 25 | 0.96 | 298 |
| GCCGGCCGCACCGGGCCGCGACGCCCGGCC | 2989 | 30 | 0.93 | 299 |
| GGAAGCGGAGACTCTATGCTTCGCC | 2990 | 25 | 0.6 | 300 |
| GGCCCAGGCCCGGGCTCCCAGGCCGGCGCC | 2991 | 30 | 0.9 | 301 |
| CCCCGTCGTACGGGCCGCTCCCGCCGCC | 2992 | 28 | 0.86 | 302 |
| CCCGCGCGAGGAGGAGCC | 2993 | 18 | 0.83 | 303 |
| ATGATCCTGACGACGGAGACCGCCGTCGTCGACAAGCC | 2994 | 38 | 0.63 | 304 |
| CCAGGTGGTACCATATGGTCCC | 2995 | 22 | 0.59 | 305 |
| AAAGGGGACACCGGGGTCCC | 2996 | 20 | 0.7 | 306 |
| CTCCGCCCTAGTGTAGTCCC | 2997 | 20 | 0.65 | 307 |
| GGCGCCGAAGGTCCCAGGTGGACCGGGCCC | 2998 | 30 | 0.8 | 308 |
| CGTGGCCGTAGGTCCGGTTCGGCCC | 2999 | 25 | 0.76 | 309 |
| GTGGTGACGGAGGCTCCCACGGCCC | 3000 | 25 | 0.76 | 310 |
| CGGGCCGCCGATGTGCGCCC | 3001 | 20 | 0.85 | 311 |
| GCTCCCCAGTGGCCGCCC | 3002 | 18 | 0.83 | 312 |
| CCTAGGCGGGTCGGAGGGCCCC | 3003 | 22 | 0.82 | 313 |
| AGGTCGCCTGTGCGCGCCCC | 3004 | 20 | 0.8 | 314 |
| AGGATGGGTAGAATAACGACCC | 3005 | 22 | 0.5 | 315 |
| CGCATGTCCCGCCTCTACCCAGGAAGATGGCACCC | 3006 | 35 | 0.66 | 316 |
| CGACAACAAAGATTATGTCACTTCTCTACC | 3007 | 30 | 0.4 | 317 |
| AGCGGGGACC | 3008 | 10 | 0.8 | 318 |
| AGGTGCACACAGTTGCGACC | 3009 | 20 | 0.6 | 319 |
| AGGTCACTCCCGACC | 3010 | 15 | 0.67 | 320 |
| CTGCGGCACC | 3011 | 10 | 0.8 | 321 |
| CCTCCTCACCTTTGCCAGAGGGACTTCGCTTGGGAACACC | 3012 | 40 | 0.6 | 322 |
| CAAGTTGTGCCCCTAGTGAGCTTGGATTAC | 3013 | 30 | 0.5 | 323 |
| TTGCTCTCTGCCATGTAC | 3014 | 18 | 0.5 | 324 |
| GGCAATTAACCATAC | 3015 | 15 | 0.4 | 325 |
| ATCCCGCGAGTGACCGCGAC | 3016 | 20 | 0.7 | 326 |
| TGTGGCCCGCGCTCCGGCAC | 3017 | 20 | 0.8 | 327 |
| GCGCCACAGGCGCAC | 3018 | 15 | 0.8 | 328 |
| CTCGAGTCGGACCAC | 3019 | 15 | 0.67 | 329 |
| GGTAACGTAATACAAGGAAACATGAGACAC | 3020 | 30 | 0.4 | 330 |
| TGCGTATTCCTAGATAGCTCCACAC | 3021 | 25 | 0.48 | 331 |
| ACCGTTACCGCCGAACGACCTCGCGGGCTCAACAC | 3022 | 35 | 0.66 | 332 |
| CGCAGTCACAGGGAGTCGCGACCGTTGCTGCATTA | 3023 | 35 | 0.6 | 333 |
| GGACGCGGACGCCGGGTA | 3024 | 18 | 0.78 | 334 |
| CCCCTAGGTAGCCAGAAGGGCGAAACCACGTGCTA | 3025 | 35 | 0.6 | 335 |
| ACGCCCTCGAGCACCCAGGCGACCCCTA | 3026 | 28 | 0.71 | 336 |
| TCTGCAACGATGGAATCAACACACTATA | 3027 | 28 | 0.39 | 337 |
| GTATCGGGGTAATGCATATA | 3028 | 20 | 0.4 | 338 |
| TTCTCAAAGAGACTTTGTTAAGGGTACCGCTGATA | 3029 | 35 | 0.4 | 339 |
| GTCAGCTCTATATTTCCTGACTTGA | 3030 | 25 | 0.4 | 340 |
| TTCTACGCCGGTTTCTGA | 3031 | 18 | 0.5 | 341 |
| TTGGAGTTTGCTCTGTGTATGA | 3032 | 22 | 0.41 | 342 |
| GACCGCCGTAGAGTACAATGACTCACGCGGCTCGA | 3033 | 35 | 0.6 | 343 |
| CGGCGGATCGCGCCCCTGGCGCGGGGCCGA | 3034 | 30 | 0.87 | 344 |
| TGCACGCGGGGCCGA | 3035 | 15 | 0.8 | 345 |
| GTCGGCCTGCACGCCGGCCACGCCCGGGGCGCCGA | 3036 | 35 | 0.86 | 346 |
| AAGCCCTGCGCAGCGGTCCGTCCGCCGA | 3037 | 28 | 0.75 | 347 |
| GCCTGGCCGGGCCTCACCGA | 3038 | 20 | 0.8 | 348 |
| GGGTCTGATGCCCGAGACCACGA | 3039 | 23 | 0.65 | 349 |
| AGGAACATCAAGTGCTAGAAGA | 3040 | 22 | 0.41 | 350 |
| CTTAGGGTCTCAGTAACCCACTTCA | 3041 | 25 | 0.48 | 351 |
| GCCTCGCGGGCGGCCCGGGTCA | 3042 | 22 | 0.86 | 352 |
| ACGGGACATTTGTTATCTCA | 3043 | 20 | 0.4 | 353 |
| TGTAGCTATGGATCA | 3044 | 15 | 0.4 | 354 |
| GGATGGCTAATTATCTGTTTACCCGTCACAAATCA | 3045 | 35 | 0.4 | 355 |
| CCAGGACAAGGAACGATTGAATGGTGCA | 3046 | 28 | 0.5 | 356 |
| GCCCACCCGTCCGGCGGACGGCCTCCTGCA | 3047 | 30 | 0.8 | 357 |
| GGATGAGGCCGAGGCAGGCAGCATCGGACCATCATGCA | 3048 | 38 | 0.63 | 358 |
| TTTCCGGCGCGCAGATCGGGCA | 3049 | 22 | 0.68 | 359 |
| CGCCCCGCGGGGCGCTCGCA | 3050 | 20 | 0.9 | 360 |
| GAGCCCAGCATAGCA | 3051 | 15 | 0.6 | 361 |
| GGCCTGTGGCTACGGGCGGCGGGCAGCA | 3052 | 28 | 0.79 | 362 |
| ACTATACGGACCGAGTCCGCCA | 3053 | 22 | 0.59 | 363 |
| ACGCGCGTTACCCCA | 3054 | 15 | 0.67 | 364 |
| TGTTGACTTAACGCCATGACCTGGTGCTGTCACCA | 3055 | 35 | 0.51 | 365 |
| AGTTCGCTTCCATAGCATTTAACCA | 3056 | 25 | 0.4 | 366 |
| TATCTAACAATGAAGTTGCACA | 3057 | 22 | 0.32 | 367 |
| AGGTGGCTACAATTGCCACA | 3058 | 20 | 0.5 | 368 |
| ATGCGTCCACGCGCCCAGGCTGTAA | 3059 | 25 | 0.64 | 369 |
| AGTCATACCACGCACATCATAA | 3060 | 22 | 0.41 | 370 |
| TACTTCTTGAATGTAATTACCAAATACGTTAGCTATGGAA | 3061 | 40 | 0.3 | 371 |
| CGCCGCGGGCTGGGCCCGGGCGCGGGCGGGCGGAA | 3062 | 35 | 0.91 | 372 |
| CCGGAGGGCTCCGACCGGAA | 3063 | 20 | 0.75 | 373 |
| ATCAAGCGACGCACGCGTGCCTCCAGAA | 3064 | 28 | 0.61 | 374 |
| CGGTCCGCACCGCAAGAA | 3065 | 18 | 0.67 | 375 |
| GCGGTATCACCTACGACATCAA | 3066 | 22 | 0.5 | 376 |
| TTCGCCCGGGCCCGTAGAGAAGGCTCGGCGTGCGGCAA | 3067 | 38 | 0.71 | 377 |
| GGGCCGGTGGGCCAA | 3068 | 15 | 0.8 | 378 |
| GCCCCTACAA | 3069 | 10 | 0.6 | 379 |
| GTTGGTGTGCACTACGACAA | 3070 | 20 | 0.5 | 380 |
| CAATGTTCGTATAACCGTCCCTAGTGTGTCTGTTACTAAA | 3071 | 40 | 0.4 | 381 |
| AGTAGCTGTACCCAACTAAA | 3072 | 20 | 0.4 | 382 |
| CGCGAGGACAACCTTTCCAATTCGGAAA | 3073 | 28 | 0.5 | 383 |
| GAAGCGGAAA | 3074 | 10 | 0.5 | 384 |

TABLE 38
Exemplary 3' RTT sequences

| | | | | RTT |
|---|---|---|---|---|
| | SEQ ID | RTT | RTT GC | Pairing |
| Sequence | NO: | length | content | NO: |
| ATGATCCTGACGACGGAGACCGCCGTCGTCGACAAGCC | 3075 | 38 | 0.63 | 1 |
| GGTAATAGCATACAGTTGCCGGTTGCAAATCTTGGAAA | 3076 | 38 | 0.421052632 | 2 |
| TGTAATCCGAGTACGCTTCGTGATAAACCGGAACAATA | 3077 | 38 | 0.421052632 | 3 |
| GATAAGTATTGATATGGAACGCATAGGGTGGTGCGATA | 3078 | 38 | 0.421052632 | 4 |
| TGCGGTGAATAGGTTGAAAGAAGGATACTGTAGACGAA | 3079 | 38 | 0.421052632 | 5 |
| GTGGAAATCATCCGCCATGAAGTATTCACACCATAACA | 3080 | 38 | 0.421052632 | 6 |
| TGGATCGGCAATATGTTTGCATGGGCTTCGTATACATA | 3081 | 38 | 0.421052632 | 7 |
| GGTAATCGCAAATGCCAAATACCATGTAGACACGGTAA | 3082 | 38 | 0.421052632 | 8 |
| TTCTGTTGGAGTGCCCGTTGGTATATGTACCTTGTATA | 3083 | 38 | 0.421052632 | 9 |
| TCCCTCTGGACGTGTAAATTTCCAAGTATACGCAGTAA | 3084 | 38 | 0.421052632 | 10 |
| GGCGGTGCCATATCCTGACCGTATACCCTGTACATACA | 3085 | 38 | 0.526315789 | 11 |
| TGTCCGTGCATTCGAGTACCTGTACTTGTCGAGGGTGA | 3086 | 38 | 0.526315789 | 12 |
| GCTCCAGAATACGCCTGAGAGAGCGAAAGCTTTCGGAA | 3087 | 38 | 0.526315789 | 13 |
| GGAGCAACACGGGTTGAGAGCAATTGATTTCTCCGCCA | 3088 | 38 | 0.526315789 | 14 |
| GTATGGGCCTGCACGTGGTAACAAGATGAGGAACGTGA | 3089 | 38 | 0.526315789 | 15 |
| GAGCCCACACATACTCGTGTTTGGTTCCCTTGACGAGA | 3090 | 38 | 0.526315789 | 16 |
| GAGTGACAGGAAACCCTCGTATGTCGGCGCACAGTATA | 3091 | 38 | 0.526315789 | 17 |
| GCCACACCACACCCGTGAGAAACGAAAGATTCCAGAGA | 3092 | 38 | 0.526315789 | 18 |
| GTGGAGCACACGAGGACTGGTTCTGGTGTACATAGGTA | 3093 | 38 | 0.526315789 | 19 |
| GTGTGGGAGGTCACAACCTCGAGTGTCGCCCTCCGGTA | 3094 | 38 | 0.631578947 | 20 |
| TGTGGGCGTACAATACTCTCGGCCGCGGACAGGGACGA | 3095 | 38 | 0.631578947 | 21 |
| GCGGTGGAGCATACCCAGTTCCGCGCGATCGATCGTGA | 3096 | 38 | 0.631578947 | 22 |
| GGGACGGAGCCATGCAAATGCCCGCATGCCTCCGAGAA | 3097 | 38 | 0.631578947 | 23 |
| GGCGCCTTTGCCATGGCGTGAACGCATAGCGGCAGAGA | 3098 | 38 | 0.631578947 | 24 |
| GGATGAGGCCGAGGCAGGCAGCATCGGACCATCATGCA | 3099 | 38 | 0.631578947 | 25 |
| GACCGCCCGTTCTGCATGTATGCGCAAGCGTCGACGCA | 3100 | 38 | 0.631578947 | 26 |
| GTCACCAAGGACCACGGCTTGGGCCTCGACAGAGCTGA | 3101 | 38 | 0.631578947 | 27 |
| TCGGTTCGGGCGAAACCCAGGAGACGCGTACCGGAAGA | 3102 | 38 | 0.631578947 | 28 |
| GTACAGAGCTCGGCCCGAGATACCGCCGCGGTGCGCCA | 3103 | 38 | 0.710526316 | 29 |
| GCCGGCAGGCCCGCAGGCGCTGTTCGGCTCAATTGGCA | 3104 | 38 | 0.710526316 | 30 |
| GCCGTGCGTCGGGCGAGGTGCCGGGAGCATGTGTGACA | 3105 | 38 | 0.710526316 | 31 |
| GAGGTGCCCGGTGCCGCTGAAAGCAGCGTTCGCCCGCA | 3106 | 38 | 0.710526316 | 32 |
| TTCGCCCGGGCCCGTAGAGAAGGCTCGGCGTGCGGCAA | 3107 | 38 | 0.710526316 | 33 |
| GGTGACGTTTCCCGTCCCTGGACGCAGCGTGGCGGGCA | 3108 | 38 | 0.710526316 | 34 |
| GCCATGCAGGCGTAACGCGGAGAGGTCCGGCCGCCGTA | 3109 | 38 | 0.710526316 | 35 |
| GGCAGCGTGCGCCCGGCTGATAGGCGGTTCTCGCAGCA | 3110 | 38 | 0.710526316 | 36 |
| GGCAACGCGCGGCAGGCGGCCGATAAAGCCTGGGCACA | 3111 | 38 | 0.710526316 | 37 |
| GGAAGGCCCGCCGACGGCTCGGGCCGCACCGCGATGCA | 3112 | 38 | 0.789473684 | 38 |
| GCCACGCGGCCGAGTCGCTCCCGGGCCGGGTGTGCGTA | 3113 | 38 | 0.789473684 | 39 |
| GCCGCGCTCCGCGCGAGGTGACGGTCCGGAGTCGGCCA | 3114 | 38 | 0.789473684 | 40 |
| GGTCTCGTGCGGAGGCCACGCGCGCGTGGTGGCGCGCA | 3115 | 38 | 0.789473684 | 41 |
| GGCCGGCCCTCGCCGCCTTCTGGGCGGCTCATCCGCCA | 3116 | 38 | 0.789473684 | 42 |
| GCCGTCTTCCCGGGCCCGCGGCCTCGGTCGACCTGGCA | 3117 | 38 | 0.789473684 | 43 |
| TTTCCGGGCTCCGCGGCGCACATCCCGGCGCCGGGCGA | 3118 | 38 | 0.789473684 | 44 |
| GTGCTCGCCGGGCACGTCCGCGCCAGCCGCGGCTTCGA | 3119 | 38 | 0.789473684 | 45 |
| GCTGCCCGGCACGCGGGCGTACCCTGGGCCCGTACGGA | 3120 | 38 | 0.789473684 | 46 |
| GCGGCCCACAGATAAGAGGCGGA | 3121 | 23 | 0.652173913 | 47 |
| TCGCATGGACCCGACGGTTGGCA | 3122 | 23 | 0.652173913 | 48 |
| GGCTTGTTGCGGGTGCGCTCGAA | 3123 | 23 | 0.652173913 | 49 |
| GGTTCGCAACCCTGCTGCGCGAA | 3124 | 23 | 0.652173913 | 50 |
| GTACCTGTCCCGCACCGGCCAAA | 3125 | 23 | 0.652173913 | 51 |
| GGGAGGAGGCCATCGCCAAACGA | 3126 | 23 | 0.652173913 | 52 |
| GACACGTAGACGGCTCGCCGAGA | 3127 | 23 | 0.652173913 | 53 |
| GGGTCTGATGCCCGAGACCACGA | 3128 | 23 | 0.652173913 | 54 |
| TGCGCAGAGACACGGACTGGGCA | 3129 | 23 | 0.652173913 | 55 |
| TGATCTGTGGCGATACATGCGCGGCCCACTGCGCCGCCCATTGCACA | 3130 | 53 | 0.622641509 | 56 |
| AACGCA | | | | |
| GCCCGACATCCATAGTGAAACAGCCACGTTGCTGTGGCGACCGTGG | 3131 | 53 | 0.622641509 | 57 |
| CCTCCGA | | | | |
| TTGGAGTAGCAATGCGTGTCGGGTTGTCCGAGCTGCGCCGTGCGTG | 3132 | 53 | 0.622641509 | 58 |
| ACCGAGA | | | | |
| TCCCTTGCGCGGCGGCAAACCTGATATGGCTCGGGACCGATACCAC | 3133 | 53 | 0.622641509 | 59 |
| GGAGCAA | | | | |
| GGTCCTCTCCACCACAACGGCCGCAATAATAGGTACCCAGGCCGCA | 3134 | 68 | 0.632352941 | 60 |
| CTCCCGTTGGCGCCACACCCAA | | | | |
| TGGTGCAGCCAGGGTCTGCTTGACTTTCGCCACCCAGCTCTTGCACA | 3135 | 68 | 0.632352941 | 61 |
| CTCCGCGTACGACGCCAGCGA | | | | |
| TGTTCCTGACGGCTGGGTAGCTTCGCCTTGCAGCGTGTACATGGAGC | 3136 | 68 | 0.632352941 | 62 |
| TTCCCGGCCTGCCACAGCCGA | | | | |
| GACGCGGAAGCCTTCCTCCTGCGTAGTCCGGTACGACCCTGCTGCGC | 3137 | 68 | 0.632352941 | 63 |
| AGAAAGGACACTGCCCGGTAA | | | | |
| GCATACCACCCATCTCGGGACACCGCCGTTCGCGCACGCTGTGGAT | 3138 | 68 | 0.632352941 | 64 |
| GGATGCCCTGATCCGCGTTGTA | | | | |
| GGTAGACCGCCTCCGGAGGCTCTTCGAGCCATCCCACGAAGCTGTC | 3139 | 68 | 0.632352941 | 65 |
| CAGTCCGATTCAGGCGAGAGGA | | | | |
| GGCTGCGCGGGCGTCCGCTTGCACCCTTGTAACGCAATCGGGCGTA | 3140 | 68 | 0.632352941 | 66 |
| AGGACGCAGTATCAGTCGGCAA | | | | |
| GGAAGACAGCCGCCACATATCCACTGCAGGGCCACCCTTTCGCTTC | 3141 | 68 | 0.632352941 | 67 |
| GTTGGCCGGTTTCGCTCCGGGA | | | | |
| GAAATCGAGCGAGGTTCCGTCGTCGGCGCAAGTGGTGGCTTCGCGT | 3142 | 68 | 0.632352941 | 68 |
| GGGTGGGTTGCCTCTTGCTGGA | | | | |
| GCGTGACCTCGCCGCTTCAAGGCCGCGCACGTCGATCGTGAGTAGA | 3143 | 83 | 0.626506024 | 69 |
| TTGAGGAAGGGCTGTCCTTGGCTCTGTCCTCCTCGGA | | | | |
| TCCCATCCAGTACAGCCCGGTCCGACTGTGCGCGATAGGTGTGAAG | 3144 | 83 | 0.626506024 | 70 |
| GCTGAGCGCGCAGTATCTCGAGGGTGTGAAGGCGGGA | | | | |
| GATAGCTTGGCCAGCAAGTGGCGCGCCCACCGGCGATCGGCATCCT | 3145 | 83 | 0.626506024 | 71 |
| GTGCCTTGGCTTCCGGTGTTGCCACAATTGGCTGCAA | | | | |
| GGCTGTGCGCGCCTTCGTCGCAGTCCGCTTCGTGGCAGTCCCTGCCA | 3146 | 83 | 0.626506024 | 72 |
| CCCTGATTGGGCATTTCTCGTGGTGTCTTTGGCCAA | | | | |
| TGGGATGACCAGAAGACGGACCATCTGCCGTCGGTCGACACTGAGT | 3147 | 83 | 0.626506024 | 73 |
| TCTGCCGACGGCCCTGCGCGTACGGGCCACTCAGTAA | | | | |
| GGCTCCGGCGACTTTCGAAGCTCCGCAAGCAGACGTGCAGGAAGAC | 3148 | 83 | 0.626506024 | 74 |
| CGGTTTGAAACGCAACGTACCGAACCGCAGGCCGCGA | | | | |
| TCCACACGGGACACCGGAGTGGCGTTGCCAGAGGCGTCGAGGCAAT | 3149 | 83 | 0.626506024 | 75 |
| ATGGCACGAGTGCCTCTTTCAAACGGCTCAGGCGCCA | | | | |
| GGCACGGCTGTGCAACGCCGCCGTCTCTGACATAGCGGCAACCGTA | 3150 | 83 | 0.626506024 | 76 |
| GTGGGTCCGTACAGAAGGACGCGATTGCCGCGTATGA | | | | |
| GGGTGTACGCTCCGCTCTCCTTGACAAGGCGGATCCCGCCGCCGTGA | 3151 | 83 | 0.626506024 | 77 |
| TCTGGTCTGTTCTGAGCGAGTCTGACGGTAGTGGGA | | | | |
| TCGCTCCCAGGTGTCCTCCTCCTCCCAGCGGGTCGCGCTGAGCCAAC | 3152 | 83 | 0.626506024 | 78 |
| AAAGCCGAGGAGAATGCTCCAATCGTAAGTCGCCCA | | | | |
| ACACCATCATCACCATCATCGTGC | 3153 | 24 | 0.5 | 79 |
| AAAGTCTCATTTGACTTA | 3154 | 18 | 0.28 | 80 |
| AAGCAGCTCACTTAA | 3155 | 15 | 0.4 | 81 |
| AAGCGTGGCATGAGTGCGCCACCGTCTG | 3156 | 28 | 0.64 | 82 |
| AAGCTCCTCAATTCCATTCACAAGA | 3157 | 25 | 0.4 | 83 |
| AAGGAAAGAGAGCCCGCCTCTCTCC | 3158 | 25 | 0.6 | 84 |
| AAGGGCGGTGGCCCG | 3159 | 15 | 0.8 | 85 |
| AAGGTAAGGTGCACAAACACCTTGC | 3160 | 25 | 0.48 | 86 |
| ACAGCACCATCTAGTGTAAA | 3161 | 20 | 0.4 | 87 |
| ACATGGAGAACAGAATATCTCTTGTGCAGA | 3162 | 30 | 0.4 | 88 |
| ACCACGACCTATATGTGATCCTTTA | 3163 | 25 | 0.4 | 89 |
| ACCAGCCGGCTTCCGCCGGC | 3164 | 20 | 0.8 | 90 |
| ACCCAAGAGGAGGGCAGA | 3165 | 18 | 0.61 | 91 |
| ACCCCACGTATCAGCAAAGTGCAACCCTTT | 3166 | 30 | 0.5 | 92 |
| ACCCCGCGGGGCGTGTGCCCATACCCCGGC | 3167 | 30 | 0.8 | 93 |
| ACCCGTGGGAGTGTCGAGCACTTGGATCAC | 3168 | 30 | 0.6 | 94 |
| ACGAGGCGGCCCTTGCGCCGCACCC | 3169 | 25 | 0.8 | 95 |
| ACGCCGATAGGGCAA | 3170 | 15 | 0.6 | 96 |
| ACGGAAGGCGCGGTCCGCGCAGGGG | 3171 | 25 | 0.8 | 97 |
| ACGGGCGCGGCCGCAACGGCGC | 3172 | 22 | 0.86 | 98 |
| ACGTGCGCGCTCAATACTCAATGTAACGTCCCACA | 3173 | 35 | 0.51 | 99 |
| ACTGAGAGGCGGGGCGCGGGTCGTTAAGGCCCCAT | 3174 | 35 | 0.69 | 100 |
| ACTGGGGCGCTGCGTACTCCCCTCC | 3175 | 25 | 0.72 | 101 |
| AGAACGGCCAAAGGT | 3176 | 15 | 0.53 | 102 |
| AGCAGCTATG | 3177 | 10 | 0.5 | 103 |
| AGCCCAGTTACTCAATTTCCTGTAATAGTG | 3178 | 30 | 0.4 | 104 |
| AGCCCATGCCCCGGC | 3179 | 15 | 0.8 | 105 |
| AGCCGCCGTGCGGCA | 3180 | 15 | 0.8 | 106 |
| AGCTACTTGTGTAAC | 3181 | 15 | 0.4 | 107 |
| AGCTAGGCCC | 3182 | 10 | 0.7 | 108 |
| AGCTCACTATAATCTGAGCT | 3183 | 20 | 0.4 | 109 |
| AGCTCGCGGAACAGCGCGAC | 3184 | 20 | 0.7 | 110 |
| AGCTTGAGATGACAGTGATCTCGGAAATTGATCTA | 3185 | 35 | 0.4 | 111 |
| AGGACCCTCGGTCGT | 3186 | 15 | 0.67 | 112 |
| AGGCAGGATCTTGAAATT | 3187 | 18 | 0.39 | 113 |
| AGGCCTGGCGTCCAGGCC | 3188 | 18 | 0.78 | 114 |
| AGGCGCGTAGGTGAC | 3189 | 15 | 0.67 | 115 |
| AGGCGGAAGACCGGCCGA | 3190 | 18 | 0.72 | 116 |
| AGGCGGTTCGATGAGCCGGCGG | 3191 | 22 | 0.73 | 117 |
| AGTCCTCAGTACCCCGAGCCGTGACGTCGGTTCGC | 3192 | 35 | 0.66 | 118 |
| AGTGTTTACCTTATCTGCTTACGTAGTACC | 3193 | 30 | 0.4 | 119 |
| ATACCCTAATGACCTGTCCTTTACTCTCGGCCGGCAGTAT | 3194 | 40 | 0.5 | 120 |
| ATAGGCGATTTCCGTCCAAG | 3195 | 20 | 0.5 | 121 |
| ATATGCATCGCTCAA | 3196 | 15 | 0.4 | 122 |
| ATCAGCGTCTTGCCTGGACC | 3197 | 20 | 0.6 | 123 |
| ATCCCTTGCTTCCCCACGGGTAAGCTCG | 3198 | 28 | 0.61 | 124 |
| ATCTCGCCACGGACACTGGTCC | 3199 | 22 | 0.64 | 125 |
| ATCTGCGTAGTTACT | 3200 | 15 | 0.4 | 126 |
| ATGAGTCCAGGCCATGTGAAAC | 3201 | 22 | 0.5 | 127 |
| ATGCAGACCTGCCACGCTAG | 3202 | 20 | 0.6 | 128 |
| ATGCCAATGGGGCAA | 3203 | 15 | 0.53 | 129 |
| ATTCATGTGTTCGATTCTCCCTCGGGCACA | 3204 | 30 | 0.5 | 130 |
| ATTGCTCAGGCGAACCCGTAGC | 3205 | 22 | 0.59 | 131 |
| CAAATCGATGATCCT | 3206 | 15 | 0.4 | 132 |
| CACCTGCCCAGCTGT | 3207 | 15 | 0.67 | 133 |
| CACGAGCTCCGCCAAGTGACCACCGATG | 3208 | 28 | 0.64 | 134 |
| CACGGCCGGGCCGCCGCTCT | 3209 | 20 | 0.85 | 135 |
| CACGTGCGCTTCGGGTTTCGTTCGCCGATT | 3210 | 30 | 0.6 | 136 |
| CACTACGGACAGGGC | 3211 | 15 | 0.67 | 137 |
| CACTATAGGTATGCAATG | 3212 | 18 | 0.39 | 138 |
| CACTGCACCGCTTCGGTCTGCGGCC | 3213 | 25 | 0.72 | 139 |
| CAGCCCCGGGACGGCCGC | 3214 | 18 | 0.89 | 140 |
| CAGCCTAGGC | 3215 | 10 | 0.7 | 141 |
| CATCGGCCGCCGGCGGTTGGGG | 3216 | 22 | 0.82 | 142 |
| CCACCGCGGCCCGGGGCAGGCT | 3217 | 22 | 0.86 | 143 |
| CCAGGAAGAAATTAGAGAGAATCTTGCTCA | 3218 | 30 | 0.4 | 144 |
| CCAGTGTCAG | 3219 | 10 | 0.6 | 145 |
| CCATATCAATCGGGGCCCAGGCGGTGTCTT | 3220 | 30 | 0.6 | 146 |
| CCCCGCCGCCCCGTCCGGCC | 3221 | 20 | 0.95 | 147 |
| CCCCGGCCCCGGGCCCGGGAGCGGCGGGCCGGGTG | 3222 | 35 | 0.94 | 148 |
| CCCGCCGGTG | 3223 | 10 | 0.9 | 149 |
| CCCGGAATCCGGCCGTACCA | 3224 | 20 | 0.7 | 150 |
| CCCGGCGCCACCCGG | 3225 | 15 | 0.93 | 151 |
| CCCGGCGCCTGAGCCCGGCCGCCGA | 3226 | 25 | 0.88 | 152 |
| CCCGGGGCCTTGGGGCTGCCTCGCC | 3227 | 25 | 0.84 | 153 |
| CCCGTAACGCCCACCCGCGAAC | 3228 | 22 | 0.73 | 154 |
| CCCGTCGCGGAGGAGGGTAG | 3229 | 20 | 0.75 | 155 |
| CCCGTGCCCTCGGTCACGCCGACTTCAC | 3230 | 28 | 0.71 | 156 |
| CCCTTGCCGG | 3231 | 10 | 0.8 | 157 |
| CCGAGGCCGAAACCGGAAACTACCA | 3232 | 25 | 0.6 | 158 |
| CCGAGGTCTAGGGTGTCCGA | 3233 | 20 | 0.65 | 159 |
| CCGATCCCGGCCCGCCCGCGCC | 3234 | 22 | 0.91 | 160 |
| CCGCACTCGGCCCGTGGGGC | 3235 | 20 | 0.85 | 161 |
| CCGCCCGTCGTGGAGGCGGGCTGCAGCT | 3236 | 28 | 0.79 | 162 |
| CCGCCGAGCGGCCACCCTAGGGCTGGCGGCGCGGG | 3237 | 35 | 0.86 | 163 |
| CCGCGAGGCTCGTCGCGCTGCCAAACTCGCCGGGCCCCGC | 3238 | 40 | 0.8 | 164 |
| CCGCGCCGCGGAGGCGGGCGGC | 3239 | 22 | 0.95 | 165 |
| CCGCGCGGAGGTTACGGC | 3240 | 18 | 0.78 | 166 |
| CCGCGCGGGCACGGCCGGGCCGCGCCTGGCGCGCCGGCGC | 3241 | 40 | 0.95 | 167 |
| CCGCGCGTGCCAGGCCCCGACGGGAGGCCCCAGCG | 3242 | 35 | 0.86 | 168 |
| CCGCGCTGGC | 3243 | 10 | 0.9 | 169 |
| CCGCTTGCGAACAAT | 3244 | 15 | 0.53 | 170 |
| CCGGCCCCTGAGGCCGGGCCCC | 3245 | 22 | 0.91 | 171 |
| CCGGCCTGGCCTTCTAAAGG | 3246 | 20 | 0.65 | 172 |
| CCGGCGCCACAGGCCGTGCCGCTGCGGGGC | 3247 | 30 | 0.87 | 173 |
| CCGGCGGACA | 3248 | 10 | 0.8 | 174 |
| CCGGCTCCGTAGCGATCGAAGCTAG | 3249 | 25 | 0.64 | 175 |
| CCGGCTCGCAGGCTGGGCCGATACCGTCGA | 3250 | 30 | 0.73 | 176 |
| CCGGGAGGTGGCCGTTGTTGCGCGCCGT | 3251 | 28 | 0.75 | 177 |
| CCGGGGAACGGCTTCCCCGGCGGCCCGGCTCGTAC | 3252 | 35 | 0.8 | 178 |
| CCGGTAGCACGTCCG | 3253 | 15 | 0.73 | 179 |
| CCGTAGTATTGACACCTACTGCCAT | 3254 | 25 | 0.48 | 180 |
| CCGTCCTCCG | 3255 | 10 | 0.8 | 181 |
| CCTATACACGGCCGCCCGCA | 3256 | 20 | 0.7 | 182 |
| CCTCGGTGACCCGCA | 3257 | 15 | 0.73 | 183 |
| CCTGAGCCTCGTGGC | 3258 | 15 | 0.73 | 184 |
| CCTTAAGCGTTATTGTCCCCTTTAA | 3259 | 25 | 0.4 | 185 |
| CCTTATCATCTTTAAGTTTC | 3260 | 20 | 0.3 | 186 |
| CGACAGGAGCGTCGCACACGCTCGGTCTCC | 3261 | 30 | 0.7 | 187 |
| CGACCTATTGGGACGCGGAGCG | 3262 | 22 | 0.68 | 188 |
| CGACTGCTCGGCGCAGCCCACAAGG | 3263 | 25 | 0.72 | 189 |
| CGAGGCCAGGGCGGC | 3264 | 15 | 0.87 | 190 |
| CGCCACCGGGTTCTCCGGTACCTGCGGCTTGGCCC | 3265 | 35 | 0.74 | 191 |
| CGCCCAGGTCCAATAGGTGAAATATGCT | 3266 | 28 | 0.5 | 192 |
| CGCCCCAGCCCGCGCCGGCCCGCGCCCAATGCCCGCGCGC | 3267 | 40 | 0.9 | 193 |
| CGCCCCGATCCTCGTGGCCGCTCGG | 3268 | 25 | 0.8 | 194 |
| CGCCCCGGGCCGGGGAGGCTGAGCGGGC | 3269 | 28 | 0.89 | 195 |
| CGCCGCGCACCGCGCGTGGCGTCCCCGG | 3270 | 28 | 0.89 | 196 |
| CGCCGGAGCGGCCGGGTA | 3271 | 18 | 0.83 | 197 |
| CGCGCCGGCCGTCGCCCAGC | 3272 | 20 | 0.9 | 198 |
| CGCGCGCGCTGCGGAGGGTGGTAGG | 3273 | 25 | 0.8 | 199 |
| CGCGCTAGCGGGACCGGATGTCCCCTGCCCTACTT | 3274 | 35 | 0.69 | 200 |
| CGCGGCGGCCGGCACCGCGTCGCGCGCTGGCGGCG | 3275 | 35 | 0.91 | 201 |
| CGCGGGAGCCGGCCGCCGGCGCCGCCCC | 3276 | 28 | 0.96 | 202 |
| CGCGTCCGCTGCCGGCGGGCGACGC | 3277 | 25 | 0.88 | 203 |
| CGCTCCGCGAGGAACCCCTCCTTCG | 3278 | 25 | 0.72 | 204 |
| CGCTCGCTCG | 3279 | 10 | 0.8 | 205 |
| CGGAGATCTTGCTAAGACAAATGCAAGATT | 3280 | 30 | 0.4 | 206 |
| CGGCAGACTCGCGCGTGGGCCGGGCCCCGTCCGGGCGACG | 3281 | 40 | 0.85 | 207 |
| CGGCCACCAGCGCGCGAT | 3282 | 18 | 0.78 | 208 |
| CGGCGCGCTGTACGAGGG | 3283 | 18 | 0.78 | 209 |
| CGGCTACGTCCCGGGACGTGCCCCGTGC | 3284 | 28 | 0.79 | 210 |
| CGGCTGGTCGCCGGG | 3285 | 15 | 0.87 | 211 |
| CGGGAGGCTGGGGCGCGGGGCCCGGCGCAC | 3286 | 30 | 0.9 | 212 |
| CGGGCCCCGCGTGCG | 3287 | 15 | 0.93 | 213 |
| CGGGCCGCACCCGACACCACCGCAA | 3288 | 25 | 0.76 | 214 |
| CGGGGAGCTCCGCTGATG | 3289 | 18 | 0.72 | 215 |
| CGGTAATGGCACTCTAACCGCCCTC | 3290 | 25 | 0.6 | 216 |
| CGGTCCGCGCGCAGCGACTTTC | 3291 | 22 | 0.73 | 217 |
| CGGTCCGGCCCGGGG | 3292 | 15 | 0.93 | 218 |
| CGGTGTTCGGGAAGATCG | 3293 | 18 | 0.61 | 219 |
| CGTAGGGCTTCAGTGTACCCGTGCA | 3294 | 25 | 0.6 | 220 |
| CGTATCATATTAAATACCAACGAGATAT | 3295 | 28 | 0.29 | 221 |
| CGTCGATGGGTCCGACGCCC | 3296 | 20 | 0.75 | 222 |
| CGTGCCGCGCGTGTC | 3297 | 15 | 0.8 | 223 |
| CGTGGGTCGCAACACGGCCCCTCAG | 3298 | 25 | 0.72 | 224 |
| CGTGGGTCGTCCGCTGTTAGGA | 3299 | 22 | 0.64 | 225 |
| CGTTCTGGCGAGCCGCGCGGCGTGCCGGGAGGGGA | 3300 | 35 | 0.8 | 226 |
| CTAAGATGTAGCCCTTGGTAATAGGTAA | 3301 | 28 | 0.39 | 227 |
| CTAGAATGACTTATGCGATAGAGTGCTA | 3302 | 28 | 0.39 | 228 |
| CTAGCACTGC | 3303 | 10 | 0.6 | 229 |
| CTATCATGCATGGCGATCCCAGGTTCGCGC | 3304 | 30 | 0.6 | 230 |
| CTCAGAGCAGCACTGAGTCGGC | 3305 | 22 | 0.64 | 231 |
| CTCCCCTCGTATCGAGCGTGATCTCTCC | 3306 | 28 | 0.61 | 232 |
| CTCCGTTCTTCGGCGCGCTGAGTCCGCCAT | 3307 | 30 | 0.67 | 233 |
| CTCGTCACTGGATGTGAAGT | 3308 | 20 | 0.5 | 234 |
| CTCTCTCTTCTGACTCGCTT | 3309 | 20 | 0.5 | 235 |
| CTGCTAGGGCGGTACGCCGGCGGGGCGCTG | 3310 | 30 | 0.8 | 236 |
| CTGGACGAGGTCGTGGCGGTCGAGTAAGGC | 3311 | 30 | 0.67 | 237 |
| CTGTACCCGTAGCCGCGGCCCCATCCGAGG | 3312 | 30 | 0.73 | 238 |
| CTTTACATCAGAATT | 3313 | 15 | 0.27 | 239 |
| GAAAGTAATTACACCTTACCTATCA | 3314 | 25 | 0.32 | 240 |
| GAAGCCGTAC | 3315 | 10 | 0.6 | 241 |
| GACGACATAAGTACCCCA | 3316 | 18 | 0.5 | 242 |
| GACGCTCGGACCGGATAG | 3317 | 18 | 0.67 | 243 |
| GACGGAGGCCGGGATGCCTTCGCCGCTCAG | 3318 | 30 | 0.73 | 244 |
| GACGGCCCGCGTGACTGCGACCCACAGC | 3319 | 28 | 0.75 | 245 |
| GACGTCGTTGTGTGC | 3320 | 15 | 0.6 | 246 |
| GAGAGATATATTCGACTCTTCCCTA | 3321 | 25 | 0.4 | 247 |
| GAGGGCGAACGACCCGTCCCTCTGCGGGTT | 3322 | 30 | 0.7 | 248 |
| GAGGTTCCCACCCGTAGT | 3323 | 18 | 0.61 | 249 |
| GAGTGCGATGCACAG | 3324 | 15 | 0.6 | 250 |
| GATAATCTTTAACAACGTGAAGAATTCATGTTTGA | 3325 | 35 | 0.29 | 251 |
| GATATGATACTGAACGCCCCTAGTGCCAATGGACC | 3326 | 35 | 0.51 | 252 |
| GATTTGATAATCTCGTATATAGAGCAGTTA | 3327 | 30 | 0.3 | 253 |
| GCAATGCACCGCCGTGGGCGCCGTATCG | 3328 | 28 | 0.71 | 254 |
| GCACATGTCCACGCCAGCAGCGCCAGGTGGCGGACAGGGA | 3329 | 40 | 0.7 | 255 |
| GCACGCTCTCGTCGGTCCGCCCTGCGCCGG | 3330 | 30 | 0.8 | 256 |
| GCAGATAATCGATAGAGT | 3331 | 18 | 0.39 | 257 |
| GCATCGGACGCTGCGACGGCGCCGGCCCCGGCAGT | 3332 | 35 | 0.8 | 258 |
| GCATGATGTGAGATCCAACGTTTATTCTTC | 3333 | 30 | 0.4 | 259 |
| GCATGCACAGGCACAATGTCTGTTT | 3334 | 25 | 0.48 | 260 |
| GCCAGGCCCCGACCGCTGGAGCGCG | 3335 | 25 | 0.84 | 261 |
| GCCCCACGCCACGGCATTTCCA | 3336 | 22 | 0.68 | 262 |
| GCCCGACAGCGCGCGCAT | 3337 | 18 | 0.78 | 263 |
| GCCCGCTCCCGCCGCCCG | 3338 | 18 | 0.94 | 264 |
| GCCGCCCCGGGGACGTCCCGTTGCC | 3339 | 25 | 0.84 | 265 |
| GCCGGAGCAGGGCCGACTGG | 3340 | 20 | 0.8 | 266 |
| GCCGTCGTGGAGGCAAGGCCTGCGT | 3341 | 25 | 0.72 | 267 |
| GCCTCCCGCTCACACTCCAACCGGCTGCGGGCACG | 3342 | 35 | 0.74 | 268 |
| GCCTCCGCCCACCGC | 3343 | 15 | 0.87 | 269 |
| GCCTGAGATTCGAAGCCGCT | 3344 | 20 | 0.6 | 270 |
| GCCTGCACCCTTGCTCCTCGACGAGGGCCCGCACCGGTCG | 3345 | 40 | 0.75 | 271 |
| GCGAAACCTCAGGTCGCCGTTTCCT | 3346 | 25 | 0.6 | 272 |
| GCGAGCTAGGGCCAAGCCTGTGGCGGTCCA | 3347 | 30 | 0.7 | 273 |
| GCGAGTGCCAGTCGCCCGGGGC | 3348 | 22 | 0.82 | 274 |
| GCGCGATGTCAGCTACACCACTTCG | 3349 | 25 | 0.6 | 275 |
| GCGCGCTGACCGGCGCGGCCACCACCCGGG | 3350 | 30 | 0.87 | 276 |
| GCGCGGCGTCTACTCCTCGCCTCTACCTCG | 3351 | 30 | 0.7 | 277 |
| GCGGAGACAGTTCTTAGATC | 3352 | 20 | 0.5 | 278 |
| GCGGATTGAACACGA | 3353 | 15 | 0.53 | 279 |
| GCGGCAAGAATGTCC | 3354 | 15 | 0.6 | 280 |
| GCGGCCTTAAGTTCGCTCTCACCCCGTCGTCACCAGGACC | 3355 | 40 | 0.65 | 281 |
| GCGTGTAACGCGCGGGCT | 3356 | 18 | 0.72 | 282 |
| GCTACGGCCGGAGTGTTC | 3357 | 18 | 0.67 | 283 |
| GCTCCGCATGGTCTCTGAACGGACGACCGG | 3358 | 30 | 0.67 | 284 |
| GCTCTCTTGGCAGCGGCTTCCCCACTTCTGGGCGC | 3359 | 35 | 0.69 | 285 |
| GCTCTTTCAGCGGCTAAATCTTAGAGCGAG | 3360 | 30 | 0.5 | 286 |
| GGACGGCCCGTACAT | 3361 | 15 | 0.67 | 287 |
| GGACTATCCCTCCATGGCGT | 3362 | 20 | 0.6 | 288 |
| GGAGGCTCGCACCGAAATCAGCGAC | 3363 | 25 | 0.64 | 289 |
| GGATCGTCCCTCAGGGGCAG | 3364 | 20 | 0.7 | 290 |
| GGATGTCCATTGGACCGCGCTTCGGTGC | 3365 | 28 | 0.64 | 291 |
| GGATTCCGGTACCCGCGGGGTGGCCTTGGA | 3366 | 30 | 0.7 | 292 |
| GGCACCCCGGCGTGCGCG | 3367 | 18 | 0.89 | 293 |
| GGCACCGGGGCACCGCCACCCCACGCGG | 3368 | 28 | 0.86 | 294 |
| GGCAGCGGGCTCGGGGACCCGTGAG | 3369 | 25 | 0.8 | 295 |
| GGCCGATGCCAGTGACCCGTTCAACGGGCCGGGCG | 3370 | 35 | 0.74 | 296 |
| GGCCGGCCCTGCCTCCGCTCCGGTGCGC | 3371 | 28 | 0.86 | 297 |
| GGCCGGGCCTCGCGGGGCGCCCGGG | 3372 | 25 | 0.96 | 298 |
| GGCCGGGCGTCGCGGCCCGGTGCGGCCGGC | 3373 | 30 | 0.93 | 299 |
| GGCGAAGCATAGAGTCTCCGCTTCC | 3374 | 25 | 0.6 | 300 |
| GGCGCCGGCCTGGGAGCCCGGGCCTGGGCC | 3375 | 30 | 0.9 | 301 |
| GGCGGCGGGAGCGGCCCGTACGACGGGG | 3376 | 28 | 0.86 | 302 |
| GGCTCCTCCTCGCGCGGG | 3377 | 18 | 0.83 | 303 |
| GGCTTGTCGACGACGGCGGTCTCCGTCGTCAGGATCAT | 3378 | 38 | 0.63 | 304 |
| GGGACCATATGGTACCACCTGG | 3379 | 22 | 0.59 | 305 |
| GGGACCCCGGTGTCCCCTTT | 3380 | 20 | 0.7 | 306 |
| GGGACTACACTAGGGCGGAG | 3381 | 20 | 0.65 | 307 |
| GGGCCCGGTCCACCTGGGACCTTCGGCGCC | 3382 | 30 | 0.8 | 308 |
| GGGCCGAACCGGACCTACGGCCACG | 3383 | 25 | 0.76 | 309 |
| GGGCCGTGGGAGCCTCCGTCACCAC | 3384 | 25 | 0.76 | 310 |
| GGGCGCACATCGGCGGCCCG | 3385 | 20 | 0.85 | 311 |
| GGGCGGCCACTGGGGAGC | 3386 | 18 | 0.83 | 312 |
| GGGGCCCTCCGACCCGCCTAGG | 3387 | 22 | 0.82 | 313 |
| GGGGCGCGCACAGGCGACCT | 3388 | 20 | 0.8 | 314 |
| GGGTCGTTATTCTACCCATCCT | 3389 | 22 | 0.5 | 315 |
| GGGTGCCATCTTCCTGGGTAGAGGCGGGACATGCG | 3390 | 35 | 0.66 | 316 |
| GGTAGAGAAGTGACATAATCTTTGTTGTCG | 3391 | 30 | 0.4 | 317 |
| GGTCCCCGCT | 3392 | 10 | 0.8 | 318 |
| GGTCGCAACTGTGTGCACCT | 3393 | 20 | 0.6 | 319 |
| GGTCGGGAGTGACCT | 3394 | 15 | 0.67 | 320 |
| GGTGCCGCAG | 3395 | 10 | 0.8 | 321 |
| GGTGTTCCCAAGCGAAGTCCCTCTGGCAAAGGTGAGGAGG | 3396 | 40 | 0.6 | 322 |
| GTAATCCAAGCTCACTAGGGGCACAACTTG | 3397 | 30 | 0.5 | 323 |
| GTACATGGCAGAGAGCAA | 3398 | 18 | 0.5 | 324 |
| GTATGGTTAATTGCC | 3399 | 15 | 0.4 | 325 |
| GTCGCGGTCACTCGCGGGAT | 3400 | 20 | 0.7 | 326 |
| GTGCCGGAGCGCGGGCCACA | 3401 | 20 | 0.8 | 327 |
| GTGCGCCTGTGGCGC | 3402 | 15 | 0.8 | 328 |
| GTGGTCCGACTCGAG | 3403 | 15 | 0.67 | 329 |
| GTGTCTCATGTTTCCTTGTATTACGTTACC | 3404 | 30 | 0.4 | 330 |
| GTGTGGAGCTATCTAGGAATACGCA | 3405 | 25 | 0.48 | 331 |
| GTGTTGAGCCCGCGAGGTCGTTCGGCGGTAACGGT | 3406 | 35 | 0.66 | 332 |
| TAATGCAGCAACGGTCGCGACTCCCTGTGACTGCG | 3407 | 35 | 0.6 | 333 |
| TACCCGGCGTCCGCGTCC | 3408 | 18 | 0.78 | 334 |
| TAGCACGTGGTTTCGCCCTTCTGGCTACCTAGGGG | 3409 | 35 | 0.6 | 335 |
| TAGGGGTCGCCTGGGTGCTCGAGGGCGT | 3410 | 28 | 0.71 | 336 |
| TATAGTGTGTTGATTCCATCGTTGCAGA | 3411 | 28 | 0.39 | 337 |
| TATATGCATTACCCCGATAC | 3412 | 20 | 0.4 | 338 |
| TATCAGCGGTACCCTTAACAAAGTCTCTTTGAGAA | 3413 | 35 | 0.4 | 339 |
| TCAAGTCAGGAAATATAGAGCTGAC | 3414 | 25 | 0.4 | 340 |
| TCAGAAACCGGCGTAGAA | 3415 | 18 | 0.5 | 341 |
| TCATACACAGAGCAAACTCCAA | 3416 | 22 | 0.41 | 342 |
| TCGAGCCGCGTGAGTCATTGTACTCTACGGCGGTC | 3417 | 35 | 0.6 | 343 |
| TCGGCCCCGCGCCAGGGGCGCGATCCGCCG | 3418 | 30 | 0.87 | 344 |
| TCGGCCCCGCGTGCA | 3419 | 15 | 0.8 | 345 |
| TCGGCGCCCCGGGCGTGGCCGGCGTGCAGGCCGAC | 3420 | 35 | 0.86 | 346 |
| TCGGCGGACGGACCGCTGCGCAGGGCTT | 3421 | 28 | 0.75 | 347 |
| TCGGTGAGGCCCGGCCAGGC | 3422 | 20 | 0.8 | 348 |
| TCGTGGTCTCGGGCATCAGACCC | 3423 | 23 | 0.65 | 349 |
| TCTTCTAGCACTTGATGTTCCT | 3424 | 22 | 0.41 | 350 |
| TGAAGTGGGTTACTGAGACCCTAAG | 3425 | 25 | 0.48 | 351 |
| TGACCCGGGCCGCCCGCGAGGC | 3426 | 22 | 0.86 | 352 |
| TGAGATAACAAATGTCCCGT | 3427 | 20 | 0.4 | 353 |
| TGATCCATAGCTACA | 3428 | 15 | 0.4 | 354 |
| TGATTTGTGACGGGTAAACAGATAATTAGCCATCC | 3429 | 35 | 0.4 | 355 |
| TGCACCATTCAATCGTTCCTTGTCCTGG | 3430 | 28 | 0.5 | 356 |
| TGCAGGAGGCCGTCCGCCGGACGGGTGGGC | 3431 | 30 | 0.8 | 357 |
| TGCATGATGGTCCGATGCTGCCTGCCTCGGCCTCATCC | 3432 | 38 | 0.63 | 358 |
| TGCCCGATCTGCGCGCCGGAAA | 3433 | 22 | 0.68 | 359 |
| TGCGAGCGCCCCGCGGGGCG | 3434 | 20 | 0.9 | 360 |
| TGCTATGCTGGGCTC | 3435 | 15 | 0.6 | 361 |
| TGCTGCCCGCCGCCCGTAGCCACAGGCC | 3436 | 28 | 0.79 | 362 |
| TGGCGGACTCGGTCCGTATAGT | 3437 | 22 | 0.59 | 363 |
| TGGGGTAACGCGCGT | 3438 | 15 | 0.67 | 364 |
| TGGTGACAGCACCAGGTCATGGCGTTAAGTCAACA | 3439 | 35 | 0.51 | 365 |
| TGGTTAAATGCTATGGAAGCGAACT | 3440 | 25 | 0.4 | 366 |
| TGTGCAACTTCATTGTTAGATA | 3441 | 22 | 0.32 | 367 |
| TGTGGCAATTGTAGCCACCT | 3442 | 20 | 0.5 | 368 |
| TTACAGCCTGGGCGCGTGGACGCAT | 3443 | 25 | 0.64 | 369 |
| TTATGATGTGCGTGGTATGACT | 3444 | 22 | 0.41 | 370 |
| TTCCATAGCTAACGTATTTGGTAATTACATTCAAGAAGTA | 3445 | 40 | 0.3 | 371 |
| TTCCGCCCGCCCGCGCCCGGGCCCAGCCCGCGGCG | 3446 | 35 | 0.91 | 372 |
| TTCCGGTCGGAGCCCTCCGG | 3447 | 20 | 0.75 | 373 |
| TTCTGGAGGCACGCGTGCGTCGCTTGAT | 3448 | 28 | 0.61 | 374 |
| TTCTTGCGGTGCGGACCG | 3449 | 18 | 0.67 | 375 |
| TTGATGTCGTAGGTGATACCGC | 3450 | 22 | 0.5 | 376 |
| TTGCCGCACGCCGAGCCTTCTCTACGGGCCCGGGCGAA | 3451 | 38 | 0.71 | 377 |
| TTGGCCCACCGGCCC | 3452 | 15 | 0.8 | 378 |
| TTGTAGGGGC | 3453 | 10 | 0.6 | 379 |
| TTGTCGTAGTGCACACCAAC | 3454 | 20 | 0.5 | 380 |
| TTTAGTAACAGACACACTAGGGACGGTTATACGAACATTG | 3455 | 40 | 0.4 | 381 |
| TTTAGTTGGGTACAGCTACT | 3456 | 20 | 0.4 | 382 |
| TTTCCGAATTGGAAAGGTTGTCCTCGCG | 3457 | 28 | 0.5 | 383 |
| TTTCCGCTTC | 3458 | 10 | 0.5 | 384 |

Tables 39 and 40 provide exemplary 5′ RTTs and 3′ RTTs, respectively. Specifically, the 5′ RTTs comprise a region of identity to a sequence on the first strand of the DMPK gene upstream of the second nick site (i.e. the 5′ RTT comprises a region of complementarity to a sequence on the second strand of the DMPK gene that is directly 3′ to nucleotide 4 of the 3′ search target sequence). The 3′ RTTs comprise a region of identity to a sequence on the second strand of the DMPK gene upstream of the first nick site (i.e. the 3′ RTT comprises a region of complementarity to a sequence on the first strand of the DMPK gene that is directly 3′ to nucleotide 4 of the 5′ search target sequence). PEgRNAs comprising these RTTs (the “clean deletion RTTs”), when paired with suitable opposite side PEgRNAs, can result in deletion of the CTG repeats in the DMPK gene without introducing exogenous sequences. Preferrably for clean deletion of the CTG repeats, both a 5′ PEgRNA and a 3′ PEgRNA comprise clean deletion RTTs. Tables 39 and 40 each contains six columns: from left to right, the first column provides the actual sequence of the RTT, the second column provides the SEQ ID NO., the third column provides the RTT length (in these RTTs, also the region of complementarity length between the RTT and the endogenous DMPK sequence), the fourth column provides the GC content of the RTT, the fifth column provides a RTT Pairing number, and the sixth column provides a Pairing spacer number. For each 5′ RTT in Table 39 having a specific Pairing 3′ spacer number, a 5′ PEgRNA comprising the 5′ RTT can be used in a dual prime editing system further comprising a 3′ PEgRNA, wherein the 3′ PEgRNA has the same 3′ spacer number (the 3′ spacer number as specified in Tables 20A-36B). For each 3′ RTT in Table 40 having a specific Pairing 5′ spacer number, a 3′ PEgRNA comprising the 3′ RTT can be used in a dual prime editing system further comprising a 5′ PEgRNA, wherein the 5′ PEgRNA has the same 5′ spacer number (the 3′ spacer number as specified in Tables 1A-19B).

TABLE 39
Exemplary 5′ RTT sequences

| | | | | | Pairing |
|---|---|---|---|---|---|
| | SEQ | | | RTT | 3′ |
| | ID | RTT | RTT GC | Pairing | Spacer |
| Sequence | NO: | length | content | NO: | NO: |
| AATAAATATCCAAACCGCCG | 3459 | 20 | 0.4 | 385 | 5 |
| AGGTCAATAAATATCCAAACCGCCG | 3460 | 25 | 0.44 | 385 | 5 |
| GGACGAGGTCAATAAATATCCAAACCG | 3461 | 30 | 0.5 | 385 | 5 |
| CCG | | | | | |
| CCGAAAAAGCGG | 3462 | 12 | 0.583333333 | 386 | 16 |
| CCCCGAAAAAGCGG | 3463 | 14 | 0.642857143 | 386 | 16 |
| ATCCCCGAAAAAGCGG | 3464 | 16 | 0.5625 | 386 | 16 |
| GGATCCCCGAAAAAGCGG | 3465 | 18 | 0.611111111 | 386 | 16 |
| CGGGATCCCCGAAAAAGCGG | 3466 | 20 | 0.65 | 386 | 16 |
| TCCAAACCGCCG | 3467 | 12 | 0.666666667 | 387 | 5 |
| TATCCAAACCGCCG | 3468 | 14 | 0.571428571 | 387 | 5 |
| AATATCCAAACCGCCG | 3469 | 16 | 0.5 | 387 | 5 |
| TAAATATCCAAACCGCCG | 3470 | 18 | 0.444444444 | 387 | 5 |
| AATAAATATCCAAACCGCCG | 3471 | 20 | 0.4 | 387 | 5 |
| ACGCTCCCCAGA | 3472 | 12 | 0.666666667 | 388 | 10 |
| AGACGCTCCCCAGA | 3473 | 14 | 0.642857143 | 388 | 10 |
| CCAGACGCTCCCCAGA | 3474 | 16 | 0.6875 | 388 | 10 |
| CGCCAGACGCTCCCCAGA | 3475 | 18 | 0.722222222 | 388 | 10 |
| CGCGCCAGACGCTCCCCAGA | 3476 | 20 | 0.75 | 388 | 10 |
| TTTGCGAACCAACGATAGGTGGGGGTG | 3647 | 40 | 0.575 | 396 | 8 |
| CGTGGAGGATGGA | | | | | |
| CATGCACAAGAAAGCTTTGCACTTTGC | 3648 | 40 | 0.45 | 397 | 9 |
| GAACCAACGATAG | | | | | |
| GGCGTCATGCACAAGAAAGCTTTGCAC | 3649 | 40 | 0.5 | 398 | 3 |
| TTTGCGAACCAAC | | | | | |
| CCGAGTAAGCAGGCAGAGATCGCGCCA | 3650 | 40 | 0.65 | 388 | 10 |
| GACGCTCCCCAGA | | | | | |
| CCCGAAAAAGCGGGTTTGGCAAAAGCA | 3651 | 40 | 0.475 | 399 | 11 |
| AATTTCCCGAGTA | | | | | |
| CGCAAGTGAGGAGGGGGGCGCGGGAT | 3652 | 40 | 0.7 | 386 | 16 |
| CCCCGAAAAAGCGG | | | | | |
| AGCAGCGCAAGTGAGGAGGGGGGCGC | 3653 | 40 | 0.675 | 400 | 4 |
| GGGATCCCCGAAAA | | | | | |
| GCGGAGCCGGCTGGGGCTCCGAGAGCA | 3654 | 40 | 0.725 | 401 | 12 |
| GCGCAAGTGAGGA | | | | | |
| GGTCAATAAATATCCAAACCGCCGAAG | 3655 | 40 | 0.6 | 402 | 17 |
| CGGGCGGAGCCGG | | | | | |
| GCGAGTCGGAGGACGAGGTCAATAAAT | 3656 | 40 | 0.55 | 385 | 5 |
| ATCCAAACCGCCG | | | | | |
| GGGTTGTTGGGGGTCCTGTAGCCTGTC | 3657 | 40 | 0.65 | 403 | 13 |
| AGCGAGTCGGAGG | | | | | |
| CGTGGATTGGGGTTGTTGGGGGTCCTG | 3658 | 40 | 0.625 | 404 | 14 |
| TAGCCTGTCAGCG | | | | | |
| TTCCCAGGCCTGCAGTTTGCCCATCCAC | 3659 | 40 | 0.625 | 405 | 6 |
| GTCAGGGCCTCA | | | | | |
| GGCTTGCTGCCTTCCCAGGCCTGCAGTT | 3660 | 40 | 0.65 | 406 | 1 |
| TGCCCATCCACG | | | | | |
| GCGTGGAGGATGGAACACGGACGGCCC | 3661 | 40 | 0.675 | 407 | 15 |
| GGCTTGCTGCCTT | | | | | |
| CAACGATAGGTGGGGGTGCGTGGAGGA | 3662 | 40 | 0.625 | 408 | 7 |
| TGGAACACGGACG | | | | | |
| CGAACCAACGATAGGTGGGGGTGCGTG | 3663 | 40 | 0.6 | 409 | 2 |
| GAGGATGGAACAC | | | | | |

TABLE 40
Exemplary 3′ RTT sequences

| | | | | | Pairing |
|---|---|---|---|---|---|
| | | | | RTT | 5′ |
| | SEQ ID | RTT | RTT GC | Pairing | Spacer |
| Sequence | NO: | length | content | NO: | NO: |
| CCCTAGAACTGTCTTCGACT | 3477 | 20 | 0.5 | 389 | 6 |
| CTGAACCCTAGAACTGTCTTCGACT | 3478 | 25 | 0.48 | 389 | 6 |
| GCTCCCTGAACCCTAGAACTGTCTTC | 3479 | 30 | 0.533333333 | 389 | 6 |
| GACT | | | | | |
| AAGACTGAGTGC | 3480 | 12 | 0.5 | 390 | 5 |
| GGAAGACTGAGTGC | 3481 | 14 | 0.571428571 | 390 | 5 |
| TTGGAAGACTGAGTGC | 3482 | 16 | 0.5 | 390 | 5 |
| CGTTGGAAGACTGAGTGC | 3483 | 18 | 0.555555556 | 390 | 5 |
| CCCGTTGGAAGACTGAGTGC | 3484 | 20 | 0.6 | 390 | 5 |
| CCAGTCCTGTGA | 3485 | 12 | 0.583333333 | 391 | 11 |
| CTCCAGTCCTGTGA | 3486 | 14 | 0.571428571 | 391 | 11 |
| AGCTCCAGTCCTGTGA | 3487 | 16 | 0.5625 | 391 | 11 |
| CCAGCTCCAGTCCTGTGA | 3488 | 18 | 0.611111111 | 391 | 11 |
| GCCCAGCTCCAGTCCTGTGA | 3489 | 20 | 0.65 | 391 | 11 |
| CGCGGCCGGCGA | 3490 | 12 | 0.916666667 | 392 | 18 |
| TCCGCGGCCGGCGA | 3491 | 14 | 0.857142857 | 392 | 18 |
| GGTCCGCGGCCGGCGA | 3492 | 16 | 0.875 | 392 | 18 |
| CGGGTCCGCGGCCGGCGA | 3493 | 18 | 0.888888889 | 392 | 18 |
| GCCGGGTCCGCGGCCGGCGA | 3494 | 20 | 0.9 | 392 | 18 |
| GAGCCGCCCGCGCTCCCTGAACCCTA | 3664 | 40 | 0.65 | 410 | 7 |
| GAACTGTCTTCGAC | | | | | |
| AACTGTCTTCGACTCCGGGGCCCCGT | 3665 | 40 | 0.6 | 411 | 8 |
| TGGAAGACTGAGTG | | | | | |
| CTTCGACTCCGGGGCCCCGTTGGAAG | 3666 | 40 | 0.7 | 412 | 9 |
| ACTGAGTGCCCGGG | | | | | |
| GAAGCCGCGCCCACCGCCTGCCAGTT | 3667 | 40 | 0.7 | 413 | 10 |
| CACAACCGCTCCGA | | | | | |
| CGCTCCGAGCGTGGGTCTCCGCCCAG | 3668 | 40 | 0.7 | 391 | 11 |
| CTCCAGTCCTGTGA | | | | | |
| TGTGATCCGGGCCCGCCCCCTAGCGG | 3669 | 40 | 0.8 | 414 | 3 |
| CCGGGGAGGGAGGG | | | | | |
| GGGGAGGGAGGGGCCGGGTCCGCGG | 3670 | 40 | 0.85 | 415 | 12 |
| CCGGCGAACGGGGCT | | | | | |
| TGGTGGCCCACGCCGGCCAACTCACC | 3671 | 40 | 0.75 | 416 | 13 |
| GCAGTCTGGCGCCG | | | | | |
| CGCTCCCTGAACCCTAGAACTGTCTTC | 3672 | 40 | 0.65 | 417 | 14 |
| GACTCCGGGGCCC | | | | | |
| CGCCCAGCTCCAGTCCTGTGATCCGG | 3673 | 40 | 0.75 | 418 | 15 |
| GCCCGCCCCCTAGC | | | | | |
| CAGTCCTGTGATCCGGGCCCGCCCCC | 3674 | 40 | 0.775 | 419 | 16 |
| TAGCGGCCGGGGAG | | | | | |
| CCCGCCCCCTAGCGGCCGGGGAGGGA | 3675 | 40 | 0.875 | 420 | 17 |
| GGGGCCGGGTCCGC | | | | | |
| TAGCGGCCGGGGAGGGAGGGGCCGG | 3676 | 40 | 0.85 | 392 | 18 |
| GTCCGCGGCCGGCGA | | | | | |
| CGGGTCCGCGGCCGGCGAACGGGGCT | 3677 | 40 | 0.75 | 421 | 19 |
| CGAAGGGTCCTTGT | | | | | |
| GGGAGGGAGGGGCCGGGTCCGCGGC | 3678 | 40 | 0.8 | 422 | 2 |
| CGGCGAACGGGGCTC | | | | | |
| ACTGTCTTCGACTCCGGGGCCCCGTT | 3679 | 40 | 0.625 | 390 | 5 |
| GGAAGACTGAGTGC | | | | | |
| AGCCGCCCGCGCTCCCTGAACCCTAG | 3680 | 40 | 0.625 | 389 | 6 |
| AACTGTCTTCGACT | | | | | |
| TCTCCGCCCAGCTCCAGTCCTGTGATC | 3681 | 40 | 0.75 | 423 | 4 |
| CGGGCCCGCCCCC | | | | | |
| GGGAGGGAGGGGCCGGGTCCGCGGC | 3682 | 40 | 0.85 | 424 | 1 |
| CGGCGAACGGGGCTC | | | | | |

Tables 41 and 42 provide exemplary 5′ RTTs and 3′ RTTs, respectively, wherein the 5′ RTT and the 3′ RTT each comprises a region of complementarity to each other. The RTTs in Tables 41 and 42 comprise endogenous DMPK sequences that flank the CTG repeats. In some cases, the RTTs also include reduced number (5) of CTG repeats. Tables 41 and 42 each includes three columns: from left to right, the first column provides the SEQ ID NO, the second column provides the actual sequence of the RTT, and the third column provides a RTT Pairing number. For each 5′ RTT in Table 41 having a specific RTT Paring number, a 5′ PEgRNA comprising the 5′ RTT can be used in a dual prime editing system further comprising a 3′ PEgRNA, wherein the 3′ PEgRNA has the same RTT Paring number as assigned in Table 42.

TABLE 41
Exemplary 5′ RTTs

| SEQ | | RTT |
|---|---|---|
| ID | | Pairing |
| NO: | Sequence | No. |
| 3537 | ACACGGACGGCCCGGCTTGCTGCCTTCCCAGGCCTGCAGTTTGCCCATCCACGTCAGGGCCTC | 393 |
| | AGCCTGGCCGAAAGAAAGAAATGGTCTGTGATCCCCCCAGCAGCAGCAGCAGCATTCCCGGC | |
| | TACAAGGACCCTTCGAGCCCCGTTCGCCGGCCGCGGACCCGGCCCCTCCCTCCCCGGCCGCTA | |
| | GGGGGCGGGCCCGGATCACAGGACTGG | |
| 3538 | CCCGGCTTGCTGCCTTCCCAGGCCTGCAGTTTGCCCATCCACGTCAGGGCCTCAGCCTGGCCG | 393 |
| | AAAGAAAGAAATGGTCTGTGATCCCCCCAGCAGCAGCAGCAGCATTCCCGGCTACAAGGACC | |
| | CTTCGAGCCCCGTTCGCCGGCCGCGGACCCGGCCCCTCCCTCCCCGGCCGCTAGGGGGCGGG | |
| | CCCGGATC | |
| 3539 | TGCCTTCCCAGGCCTGCAGTTTGCCCATCCACGTCAGGGCCTCAGCCTGGCCGAAAGAAAGA | 393 |
| | AATGGTCTGTGATCCCCCCAGCAGCAGCAGCAGCATTCCCGGCTACAAGGACCCTTCGAGCC | |
| | CCGTTCGCCGGCCGCGGACCCGGCCCCTCCCTCCCCGGCCGCTAGGGGGCG | |
| 3540 | GGCCTGCAGTTTGCCCATCCACGTCAGGGCCTCAGCCTGGCCGAAAGAAAGAAATGGTCTGT | 393 |
| | GATCCCCCCAGCAGCAGCAGCAGCATTCCCGGCTACAAGGACCCTTCGAGCCCCGTTCGCCG | |
| | GCCGCGGACCCGGCCCCTCCCTCCCCGGCCG | |
| 3541 | TTGCCCATCCACGTCAGGGCCTCAGCCTGGCCGAAAGAAAGAAATGGTCTGTGATCCCCCCA | 393 |
| | GCAGCAGCAGCAGCATTCCCGGCTACAAGGACCCTTCGAGCCCCGTTCGCCGGCCGCGGACC | |
| | CGGCCCCTCCC | |
| 3542 | ACGTCAGGGCCTCAGCCTGGCCGAAAGAAAGAAATGGTCTGTGATCCCCCCAGCAGCAGCAG | 393 |
| | CAGCATTCCCGGCTACAAGGACCCTTCGAGCCCCGTTCGCCGGCCGCGGACCC | |
| 3543 | CTCAGCCTGGCCGAAAGAAAGAAATGGTCTGTGATCCCCCCAGCAGCAGCAGCAGCATTCCC | 393 |
| | GGCTACAAGGACCCTTCGAGCCCCGTTCGCCGG | |
| 3544 | CCGAAAGAAAGAAATGGTCTGTGATCCCCCCAGCAGCAGCAGCAGCATTCCCGGCTACAAGG | 393 |
| | ACCCTTCGAGCCC | |
| 3545 | GAAATGGTCTGTGATCCCCCCAGCAGCAGCAGCAGCATTCCCGGCTACAAGGACC | 393 |
| 3546 | GTGATCCCCCCAGCAGCAGCAGCAGCATTCCCGGC | 393 |
| 3547 | ACACGGACGGCCCGGCTTGCTGCCTTCCCAGGCCTGCAGTTTGCCCATCCACGTCAGGGCCTC | 394 |
| | AGCCTGGCCGAAAGAAAGAAATGGTCTGTGATCCCCCCATTCCCGGCTACAAGGACCCTTCG | |
| | AGCCCCGTTCGCCGGCCGCGGACCCGGCCCCTCCCTCCCCGGCCGCTAGGGGGCGGGCCCGG | |
| | ATCACAGGACTGG | |
| 3548 | CCCGGCTTGCTGCCTTCCCAGGCCTGCAGTTTGCCCATCCACGTCAGGGCCTCAGCCTGGCCG | 394 |
| | AAAGAAAGAAATGGTCTGTGATCCCCCCATTCCCGGCTACAAGGACCCTTCGAGCCCCGTTC | |
| | GCCGGCCGCGGACCCGGCCCCTCCCTCCCCGGCCGCTAGGGGGCGGGCCCGGATC | |
| 3549 | TGCCTTCCCAGGCCTGCAGTTTGCCCATCCACGTCAGGGCCTCAGCCTGGCCGAAAGAAAGA | 394 |
| | AATGGTCTGTGATCCCCCCATTCCCGGCTACAAGGACCCTTCGAGCCCCGTTCGCCGGCCGCG | |
| | GACCCGGCCCCTCCCTCCCCGGCCGCTAGGGGGCG | |
| 3550 | GGCCTGCAGTTTGCCCATCCACGTCAGGGCCTCAGCCTGGCCGAAAGAAAGAAATGGTCTGT | 394 |
| | GATCCCCCCATTCCCGGCTACAAGGACCCTTCGAGCCCCGTTCGCCGGCCGCGGACCCGGCCC | |
| | CTCCCTCCCCGGCCG | |
| 3551 | TTGCCCATCCACGTCAGGGCCTCAGCCTGGCCGAAAGAAAGAAATGGTCTGTGATCCCCCCA | 394 |
| | TTCCCGGCTACAAGGACCCTTCGAGCCCCGTTCGCCGGCCGCGGACCCGGCCCCTCCC | |
| 3552 | ACGTCAGGGCCTCAGCCTGGCCGAAAGAAAGAAATGGTCTGTGATCCCCCCATTCCCGGCTA | 394 |
| | CAAGGACCCTTCGAGCCCCGTTCGCCGGCCGCGGACCC | |
| 3553 | CTCAGCCTGGCCGAAAGAAAGAAATGGTCTGTGATCCCCCCATTCCCGGCTACAAGGACCCT | 394 |
| | TCGAGCCCCGTTCGCCGG | |
| 3554 | CCGAAAGAAAGAAATGGTCTGTGATCCCCCCATTCCCGGCTACAAGGACCCTTCGAGCCC | 394 |
| 3555 | GAAATGGTCTGTGATCCCCCCATTCCCGGCTACAAGGACC | 394 |
| 3556 | GTGATCCCCCCATTCCCGGC | 394 |
| 3557 | ACACGGACGGCCCGGCTTGCTGCCTTCCCAGGCCTGCAGTTTGCCCATCCACGTCAGGGCCTC | 396 |
| | AGCCTGGCCGAAAGAAAGAAATGGTCTGTGATCCCCC | |
| 3558 | CCCGGCTTGCTGCCTTCCCAGGCCTGCAGTTTGCCCATCCACGTCAGGGCCTCAGCCTGGCCG | 396 |
| | AAAGAAAGAAATGGTCTGTGATCCCCC | |
| 3559 | TGCCTTCCCAGGCCTGCAGTTTGCCCATCCACGTCAGGGCCTCAGCCTGGCCGAAAGAAAGA | 396 |
| | AATGGTCTGTGATCCCCC | |
| 3560 | GGCCTGCAGTTTGCCCATCCACGTCAGGGCCTCAGCCTGGCCGAAAGAAAGAAATGGTCTGT | 396 |
| | GATCCCCC | |
| 3561 | TTGCCCATCCACGTCAGGGCCTCAGCCTGGCCGAAAGAAAGAAATGGTCTGTGATCCCCC | 396 |
| 3562 | ACGTCAGGGCCTCAGCCTGGCCGAAAGAAAGAAATGGTCTGTGATCCCCC | 396 |
| 3563 | CTCAGCCTGGCCGAAAGAAAGAAATGGTCTGTGATCCCCC | 396 |
| 3564 | CCGAAAGAAAGAAATGGTCTGTGATCCCCC | 396 |
| 3565 | GAAATGGTCTGTGATCCCCC | 396 |
| 3566 | GTGATCCCCC | 396 |
| 3567 | CATTCCCGGCTACAAGGACCCTTCGAGCCCCGTTCGCCGGCCGCGGACCCGGCCCCTCCCTCC | 395 |
| | CCGGCCGCTAGGGGGCGGGCCCGGATCACAGGACTGG | |
| 3568 | CATTCCCGGCTACAAGGACCCTTCGAGCCCCGTTCGCCGGCCGCGGACCCGGCCCCTCCCTCC | 395 |
| | CCGGCCGCTAGGGGGCGGGCCCGGATC | |
| 3569 | CATTCCCGGCTACAAGGACCCTTCGAGCCCCGTTCGCCGGCCGCGGACCCGGCCCCTCCCTCC | 395 |
| | CCGGCCGCTAGGGGGCG | |
| 3570 | CATTCCCGGCTACAAGGACCCTTCGAGCCCCGTTCGCCGGCCGCGGACCCGGCCCCTCCCTCC | 395 |
| | CCGGCCG | |
| 3571 | CATTCCCGGCTACAAGGACCCTTCGAGCCCCGTTCGCCGGCCGCGGACCCGGCCCCTCCC | 395 |
| 3572 | CATTCCCGGCTACAAGGACCCTTCGAGCCCCGTTCGCCGGCCGCGGACCC | 395 |
| 3573 | CATTCCCGGCTACAAGGACCCTTCGAGCCCCGTTCGCCGG | 395 |
| 3574 | CATTCCCGGCTACAAGGACCCTTCGAGCCC | 395 |
| 3575 | CATTCCCGGCTACAAGGACC | 395 |
| 3576 | CAGCATTCCCGGC | 395 |

TABLE 42
3′ RTTs

| | | RTT |
|---|---|---|
| SEQ ID | | Pairing |
| NO: | Sequence | No. |
| 3497 | CCAGTCCTGTGATCCGGGCCCGCCCCCTAGCGGCCGGGGAGGGAGGGGCCGGGTCCGCGGC | 393 |
| | CGGCGAACGGGGCTCGAAGGGTCCTTGTAGCCGGGAATGCTGCTGCTGCTGCTGGGGGGAT | |
| | CACAGACCATTTCTTTCTTTCGGCCAGGCTGAGGCCCTGACGTGGATGGGCAAACTGCAGGC | |
| | CTGGGAAGGCAGCAAGCCGGGCCGTCCGTGT | |
| 3498 | GATCCGGGCCCGCCCCCTAGCGGCCGGGGAGGGAGGGGCCGGGTCCGCGGCCGGCGAACGG | 393 |
| | GGCTCGAAGGGTCCTTGTAGCCGGGAATGCTGCTGCTGCTGCTGGGGGGATCACAGACCATT | |
| | TCTTTCTTTCGGCCAGGCTGAGGCCCTGACGTGGATGGGCAAACTGCAGGCCTGGGAAGGCA | |
| | GCAAGCCGGG | |
| 3499 | CGCCCCCTAGCGGCCGGGGAGGGAGGGGCCGGGTCCGCGGCCGGCGAACGGGGCTCGAAG | 393 |
| | GGTCCTTGTAGCCGGGAATGCTGCTGCTGCTGCTGGGGGGATCACAGACCATTTCTTTCTTTC | |
| | GGCCAGGCTGAGGCCCTGACGTGGATGGGCAAACTGCAGGCCTGGGAAGGCA | |
| 3500 | CGGCCGGGGAGGGAGGGGCCGGGTCCGCGGCCGGCGAACGGGGCTCGAAGGGTCCTTGTAG | 393 |
| | CCGGGAATGCTGCTGCTGCTGCTGGGGGGATCACAGACCATTTCTTTCTTTCGGCCAGGCTG | |
| | AGGCCCTGACGTGGATGGGCAAACTGCAGGCC | |
| 3501 | GGGAGGGGCCGGGTCCGCGGCCGGCGAACGGGGCTCGAAGGGTCCTTGTAGCCGGGAATGC | 393 |
| | TGCTGCTGCTGCTGGGGGGATCACAGACCATTTCTTTCTTTCGGCCAGGCTGAGGCCCTGAC | |
| | GTGGATGGGCAA | |
| 3502 | GGGTCCGCGGCCGGCGAACGGGGCTCGAAGGGTCCTTGTAGCCGGGAATGCTGCTGCTGCT | 393 |
| | GCTGGGGGGATCACAGACCATTTCTTTCTTTCGGCCAGGCTGAGGCCCTGACGT | |
| 3503 | CCGGCGAACGGGGCTCGAAGGGTCCTTGTAGCCGGGAATGCTGCTGCTGCTGCTGGGGGGA | 393 |
| | TCACAGACCATTTCTTTCTTTCGGCCAGGCTGAG | |
| 3504 | GGGCTCGAAGGGTCCTTGTAGCCGGGAATGCTGCTGCTGCTGCTGGGGGGATCACAGACCAT | 393 |
| | TTCTTTCTTTCGG | |
| 3505 | GGTCCTTGTAGCCGGGAATGCTGCTGCTGCTGCTGGGGGGATCACAGACCATTTC | 393 |
| 3506 | GCCGGGAATGCTGCTGCTGCTGCTGGGGGGATCAC | 393 |
| 3507 | CCAGTCCTGTGATCCGGGCCCGCCCCCTAGCGGCCGGGGAGGGAGGGGCCGGGTCCGCGGC | 394 |
| | CGGCGAACGGGGCTCGAAGGGTCCTTGTAGCCGGGAATGGGGGGATCACAGACCATTTCTT | |
| | TCTTTCGGCCAGGCTGAGGCCCTGACGTGGATGGGCAAACTGCAGGCCTGGGAAGGCAGCA | |
| | AGCCGGGCCGTCCGTGT | |
| 3508 | GATCCGGGCCCGCCCCCTAGCGGCCGGGGAGGGAGGGGCCGGGTCCGCGGCCGGCGAACGG | 394 |
| | GGCTCGAAGGGTCCTTGTAGCCGGGAATGGGGGGATCACAGACCATTTCTTTCTTTCGGCCA | |
| | GGCTGAGGCCCTGACGTGGATGGGCAAACTGCAGGCCTGGGAAGGCAGCAAGCCGGG | |
| 3509 | CGCCCCCTAGCGGCCGGGGAGGGAGGGGCCGGGTCCGCGGCCGGCGAACGGGGCTCGAAG | 394 |
| | GGTCCTTGTAGCCGGGAATGGGGGGATCACAGACCATTTCTTTCTTTCGGCCAGGCTGAGGC | |
| | CCTGACGTGGATGGGCAAACTGCAGGCCTGGGAAGGCA | |
| 3510 | CGGCCGGGGAGGGAGGGGCCGGGTCCGCGGCCGGCGAACGGGGCTCGAAGGGTCCTTGTAG | 394 |
| | CCGGGAATGGGGGGATCACAGACCATTTCTTTCTTTCGGCCAGGCTGAGGCCCTGACGTGGA | |
| | TGGGCAAACTGCAGGCC | |
| 3511 | GGGAGGGGCCGGGTCCGCGGCCGGCGAACGGGGCTCGAAGGGTCCTTGTAGCCGGGAATGG | 394 |
| | GGGGATCACAGACCATTTCTTTCTTTCGGCCAGGCTGAGGCCCTGACGTGGATGGGCAA | |
| 3512 | GGGTCCGCGGCCGGCGAACGGGGCTCGAAGGGTCCTTGTAGCCGGGAATGGGGGGATCACA | 394 |
| | GACCATTTCTTTCTTTCGGCCAGGCTGAGGCCCTGACGT | |
| 3513 | CCGGCGAACGGGGCTCGAAGGGTCCTTGTAGCCGGGAATGGGGGGATCACAGACCATTTCT | 394 |
| | TTCTTTCGGCCAGGCTGAG | |
| 3514 | GGGCTCGAAGGGTCCTTGTAGCCGGGAATGGGGGGATCACAGACCATTTCTTTCTTTCGG | 394 |
| 3515 | GGTCCTTGTAGCCGGGAATGGGGGGATCACAGACCATTTC | 394 |
| 3516 | GCCGGGAATGGGGGGATCAC | 394 |
| 3517 | GCCGGGAATG | 395 |
| 3518 | GGTCCTTGTAGCCGGGAATG | 395 |
| 3519 | CCAGTCCTGTGATCCGGGCCCGCCCCCTAGCGGCCGGGGAGGGAGGGGCCGGGTCCGCGGC | 395 |
| | CGGCGAACGGGGCTCGAAGGGTCCTTGTAGCCGGGAATG | |
| 3520 | GATCCGGGCCCGCCCCCTAGCGGCCGGGGAGGGAGGGGCCGGGTCCGCGGCCGGCGAACGG | 395 |
| | GGCTCGAAGGGTCCTTGTAGCCGGGAATG | |
| 3521 | CGCCCCCTAGCGGCCGGGGAGGGAGGGGCCGGGTCCGCGGCCGGCGAACGGGGCTCGAAG | 395 |
| | GGTCCTTGTAGCCGGGAATG | |
| 3522 | CGGCCGGGGAGGGAGGGGCCGGGTCCGCGGCCGGCGAACGGGGCTCGAAGGGTCCTTGTAG | 395 |
| | CCGGGAATG | |
| 3523 | GGGAGGGGCCGGGTCCGCGGCCGGCGAACGGGGCTCGAAGGGTCCTTGTAGCCGGGAATG | 395 |
| 3524 | GGGTCCGCGGCCGGCGAACGGGGCTCGAAGGGTCCTTGTAGCCGGGAATG | 395 |
| 3525 | CCGGCGAACGGGGCTCGAAGGGTCCTTGTAGCCGGGAATG | 395 |
| 3526 | GGGCTCGAAGGGTCCTTGTAGCCGGGAATG | 395 |
| 3527 | GGGGGATCACAGACCATTTCTTTCTTTCGGCCAGGCTGAGGCCCTGACGTGGATGGGCAAAC | 396 |
| | TGCAGGCCTGGGAAGGCAGCAAGCCGGGCCGTCCGTGT | |
| 3528 | GGGGGATCACAGACCATTTCTTTCTTTCGGCCAGGCTGAGGCCCTGACGTGGATGGGCAAAC | 396 |
| | TGCAGGCCTGGGAAGGCAGCAAGCCGGG | |
| 3529 | GGGGGATCACAGACCATTTCTTTCTTTCGGCCAGGCTGAGGCCCTGACGTGGATGGGCAAAC | 396 |
| | TGCAGGCCTGGGAAGGCA | |
| 3530 | GGGGGATCACAGACCATTTCTTTCTTTCGGCCAGGCTGAGGCCCTGACGTGGATGGGCAAAC | 396 |
| | TGCAGGCC | |
| 3531 | GGGGGATCACAGACCATTTCTTTCTTTCGGCCAGGCTGAGGCCCTGACGTGGATGGGCAA | 396 |
| 3532 | GGGGGATCACAGACCATTTCTTTCTTTCGGCCAGGCTGAGGCCCTGACGT | 396 |
| 3533 | GGGGGATCACAGACCATTTCTTTGCCGGGAATGCTGCTGCTGCTGCTGGGGGGATCACCTTT | 396 |
| | CGGCCAGGCTGAG | |
| 3534 | GGGGGATCACAGACCATTTCTTTCTTTCGG | 396 |
| 3535 | GGGGGATCACAGACCATTTC | 396 |
| 3536 | GGGGGATCAC | 396 |

The 5′ and 3′ PEgRNAs as exemplified and/or comprise components provided in Tables 1A-42 can comprise, from 5′ to 3′, the spacer, the gRNA core, the edit template, and the PBS. The 3′ end of the edit template can be contiguous with the 5′ end of the PBS. The PEgRNA can comprise multiple RNA molecules (e.g., a crRNA containing the PEgRNA spacer and a tracrRNA containing the extension arm) or can be a single gRNA molecule. Any PEgRNA exemplified in Tables 1A-42 may comprise, or further comprise, a 3′ motif at the 3′ end of the extension arm, for example, a hairpin-forming motif or a series of 1, 2, 3, 4, 5, 6, 7 or more U nucleotides. In some embodiments, the PEgRNA comprises 4 U nucleotides at its 3′ end. Without being bound by theory, such 3′ motifs are believed to increase PEgRNA stability. The PEgRNA may alternatively or additionally comprise one or more chemical modifications, such as phosphorothioate (PS) bond(s), 2′-O-methylated (2′-Ome) nucleotides, or a combination thereof. In some embodiments, the PEgRNA comprise 3′ mN*mN*mN*N and 5′mN*mN*mN*modifications, where m indicates that the nucleotide contains a 2′-O-Me modification and a * indicates the presence of a phosphorothioate bond. PEgRNA sequences exemplified in Tables 1-84 may alternatively be adapted for expression from a DNA template, for example, by including a 5′ terminal G if the spacer of the PEgRNA begins with another nucleotide, by including 6 or 7 U nucleotides at the 3′ end of the extension arm, or both. Such expression-adapted sequences may further comprise a hairpin-forming motif between the PBS and the 3′ terminal U series. In some embodiments, a prime editing system comprises one or more polynucleotides encoding one or more prime editor polypeptides, wherein activity of the prime editing system may be temporally regulated by controlling the timing in which the vectors are delivered. For example, in some embodiments, a polynucleotide encoding the prime editor and a polynucleotide encoding a PEgRNA or both PEgRNAs may be delivered simultaneously. For example, in some embodiments, a polynucleotide encoding the prime editor and a polynucleotide encoding a PEgRNA or both PEgRNAs may be delivered sequentially.

In some embodiments, a polynucleotide encoding a component of a prime editing system may further comprise an element that is capable of modifying the intracellular half-life of the polynucleotide and/or modulating translational control. In some embodiments, the polynucleotide is a RNA, for example, an mRNA. In some embodiments, the half-life of the polynucleotide, e.g., the RNA may be increased. In some embodiments, the half-life of the polynucleotide, e.g., the RNA may be decreased. In some embodiments, the element may be capable of increasing the stability of the polynucleotide, e.g., the RNA. In some embodiments, the element may be capable of decreasing the stability of the polynucleotide, e.g., the RNA. In some embodiments, the element may be within the 3′ UTR of the RNA. In some embodiments, the element may include a polyadenylation signal (PA). In some embodiments, the element may include a cap, e.g., an upstream mRNA or PEgRNA end. In some embodiments, the RNA may comprise no PA such that it is subject to quicker degradation in the cell after transcription.

In some embodiments, the element may include at least one AU-rich element (ARE). The AREs may be bound by ARE binding proteins (ARE-BPs) in a manner that is dependent upon tissue type, cell type, timing, cellular localization, and environment. In some embodiments the destabilizing element may promote RNA decay, affect RNA stability, or activate translation. In some embodiments, the ARE may comprise 50 to 150 nucleotides in length. In some embodiments, the ARE may comprise at least one copy of the sequence AUUUA. In some embodiments, at least one ARE may be added to the 3′ UTR of the RNA. In some embodiments, the element may be a Woodchuck Hepatitis Virus Posttranscriptional Regulatory Element (WPRE). In further embodiments, the element is a modified and/or truncated WPRE sequence that is capable of enhancing expression from the transcript. In some embodiments, the WPRE or equivalent may be added to the 3′ UTR of the RNA. In some embodiments, the element may be selected from other RNA sequence motifs that are enriched in either fast- or slow-decaying transcripts. In some embodiments, the polynucleotide, e.g., a vector, encoding the PE or the PEgRNA may be self-destroyed via cleavage of a target sequence present on the polynucleotide, e.g., a vector. The cleavage may prevent continued transcription of a PE or a PEgRNA.

Polynucleotides encoding prime editing composition components can be DNA, RNA, or any combination thereof. In some embodiments, a polynucleotide encoding a prime editing composition component is an expression construct. In some embodiments, a polynucleotide encoding a prime editing composition component is a vector. In some embodiments, the vector is a DNA vector. In some embodiments, the vector is a plasmid. In some embodiments, the vector is a virus vector, e.g., a retroviral vector, adenoviral vector, lentiviral vector, herpesvirus vector, or an adeno-associated virus vector (AAV).

In some embodiments, polynucleotides encoding polypeptide components of a prime editing composition are codon optimized by replacing at least one codon (e.g., about or more than about 1, 2, 3, 4, 5, 10, 15, 20, 25, 50, or more codons) of the native sequence with codons that are more frequently or most frequently used in the genes of that host cell while maintaining the native amino acid sequence. In some embodiments, a polynucleotide encoding a polypeptide component of a prime editing composition are operably linked to one or more expression regulatory elements, for example, a promoter, a 3′ UTR, a 5′ UTR, or any combination thereof. In some embodiments, a polynucleotide encoding a prime editing composition component is a messenger RNA (mRNA). In some embodiments, the mRNA comprises a Cap at the 5′ end and/or a poly A tail at the 3′ end.

### Pharmaceutical Compositions

Disclosed herein are pharmaceutical compositions comprising any of the prime editing composition components, for example, prime editors, fusion proteins, polynucleotides encoding prime editor polypeptides, PEgRNAs, and/or prime editing complexes described herein.

The term “pharmaceutical composition”, as used herein, refers to a composition formulated for pharmaceutical use. In some embodiments, the pharmaceutical composition further comprises a pharmaceutically acceptable carrier. In some embodiments, the pharmaceutical composition comprises additional agents, e.g., for specific delivery, increasing half-life, or other therapeutic compounds.

In some embodiments, a pharmaceutically-acceptable carrier comprises any vehicle, such as a liquid or solid filler, diluent, excipient, manufacturing aid (e.g., lubricant, talc magnesium, calcium or zinc stearate, or steric acid), or solvent encapsulating material, involved in carrying or transporting the compound from one site (e.g., the delivery site) of the body, to another site (e.g., organ, tissue or portion of the body). A pharmaceutically acceptable carrier is “acceptable” in the sense of being compatible with the other ingredients of the formulation and not injurious to the tissue of the subject (e.g., physiologically compatible, sterile, physiologic pH, etc.)

Formulations of the pharmaceutical compositions described herein can be prepared by any method known or hereafter developed in the art of pharmacology. In general, such preparatory methods include the step of bringing the active ingredient(s) into association with an excipient and/or one or more other accessory ingredients, and then, if necessary and/or desirable, shaping and/or packaging the product into a desired single- or multi-dose unit. Pharmaceutical formulations can additionally comprise a pharmaceutically acceptable excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired.

### Methods of Editing

The methods and compositions disclosed herein can be used to edit a target gene of interest by dual prime editing.

In some embodiments, the dual prime editing method comprises contacting a target gene, e.g., a DMPK gene, with a first PEgRNA, a second PEgRNA and a prime editor (PE) polypeptide described herein. In some embodiments, the target gene is double stranded, and comprises two strands of DNA complementary to each other. In some embodiments, the contacting with the two PEgRNAs and the contacting with a prime editor are performed sequentially. In some embodiments, the contacting with a prime editor is performed after the contacting with the two PEgRNAs. In some embodiments, the contacting with the two PEgRNAs is performed after the contacting with a prime editor. In some embodiments, the contacting with the two PEgRNAs is performed simultaneously either prior to or after contacting with a prime editor. In some embodiments, the contacting with the two PEgRNAs is performed sequentially either prior to or after contacting with a prime editor. In some embodiments, the contacting with the two PEgRNAs, and the contacting with a prime editor are performed simultaneously. In some embodiments, the two PEgRNAs and the prime editor are associated in complexes prior to contacting a target gene.

In some embodiments, contacting the target gene with the prime editing composition results in binding of the first PEgRNA to a first strand of the target gene, e.g., a DMPK gene. In some embodiments, contacting the target gene with the prime editing composition results in binding of the second PEgRNA to a second strand of the target gene, e.g., a DMPK gene. In some embodiments, contacting the target gene with the prime editing composition results in binding of the first PEgRNA to a first strand of the target gene and binding of the second PEgRNA to a second strand of the target gene, e.g., a DMPK gene. In some embodiments, contacting the target gene with the prime editing composition results in binding of the first PEgRNA to a first search target sequence on the first strand of the target gene upon contacting with the first PEgRNA. In some embodiments, contacting the target gene with the prime editing composition results in binding of the second PEgRNA to a second search target sequence on the second strand of the target gene upon contacting with the second PEgRNA. In some embodiments, contacting the target gene with the prime editing composition results in binding of the first PEgRNA to a first search target sequence on the first strand of the target gene upon contacting with the first PEgRNA and binding of the second PEgRNA to a second search target sequence on the second strand of the target gene upon contacting with the second PEgRNA. In some embodiments, contacting the target gene with the prime editing composition results in binding of a first spacer of the first PEgRNA to a first search target sequence on the first strand of the target gene upon said contacting of the first PEgRNA. In some embodiments, contacting the target gene with the prime editing composition results in binding of a second spacer of the second PEgRNA to a second search target sequence on the second strand of the target gene upon said contacting of the second PEgRNA. In some embodiments, contacting the target gene with the prime editing composition results in binding of a first spacer of the first PEgRNA to a first search target sequence on the first strand of the target gene upon said contacting of the first PEgRNA and binding of a second spacer of the second PEgRNA to a second search target sequence on the second strand of the target gene upon said contacting of the second PEgRNA.

In some embodiments, contacting the target gene with the prime editing composition results in binding of the prime editor to the target gene, e.g., the target DMPK gene, upon the contacting of the PE composition with the target gene. In some embodiments, a DNA binding domain of a prime editor associates with either a first PEgRNA and/or a second PEgRNA. In some embodiments, a prime editor associated with a first PEgRNA binds the first strand of a target gene, e.g., a DMPK gene, as directed by the first PEgRNA. In some embodiments, a prime editor associated with a second PEgRNA binds the second strand of a target gene, e.g., a DMPK gene, as directed by the second PEgRNA. In some embodiments, a prime editor associated with a first PEgRNA binds the first strand of a target gene as directed by the first PEgRNA, and a prime editor associated with a second PEgRNA binds the second strand of the target gene as directed by the second PEgRNA.

In some embodiments, a first PEgRNA directs a prime editor to generate a nick on the second strand of a target gene. In some embodiments, a second PEgRNA directs a prime editor to generate a nick on the first strand of a target gene. In some embodiments, a first PEgRNA directs a prime editor to generate a first nick on the second strand of a target gene, and a second PEgRNA directs a prime editor to generate a second nick on the first strand of a target gene, thereby generating an inter-nick duplex (IND) between the position of the first nick and the position of the second nick on the target gene. In some embodiments, the DNA binding domain of the prime editor is a Cas domain. In some embodiments, the DNA binding domain of the prime editor is a Cas9. In some embodiments, the DNA binding domain of the prime editor is a Cas9 nickase.

In some embodiments, contacting the target gene with the prime editing composition results in hybridization of the PEgRNA (e.g., the first PEgRNA and/or the second PEgRNA) with the 3′ end of the nicked single-stranded DNA, thereby priming DNA polymerization by a DNA polymerase domain of the prime editor. In some embodiments, the free 3′ end of the single-stranded DNA generated at the nick site hybridizes to a primer binding site sequence (PBS) of the contacted PEgRNA, thereby priming DNA polymerization. In some embodiments, the DNA polymerization is reverse transcription catalyzed by a reverse transcriptase domain of the prime editor. In some embodiments, the method comprises contacting the target gene with a DNA polymerase, e.g., a reverse transcriptase, as a part of a prime editor fusion protein or prime editing complex (in cis), or as a separate protein (in trans).

In some embodiments, contacting the target gene with the prime editing composition generates an overlap duplex (OD) or replacement duplex (RD) that replaces the IND. In some embodiments, the OD or RD comprises one or more intended nucleotide edits compared to the endogenous sequence of the target gene, e.g., a DMPK gene. In some embodiments, the intended nucleotide edits are incorporated in the target gene by replacement of the IND by the OD or RD. In some embodiments, the intended nucleotide edits are incorporated in the target gene by excision of the IND and DNA repair. In some embodiments, excision of the 5′ single stranded DNA of the edit strand generated at the nick site is by a flap endonuclease. In some embodiments, the flap nuclease is FEN1. In some embodiments, the method further comprises contacting the target gene with a flap endonuclease. In some embodiments, the flap endonuclease is provided as a part of a prime editor fusion protein. In some embodiments, the flap endonuclease is provided in trans.

In some embodiments, the target gene, e.g., a DMPK gene, is in a cell. Accordingly, also provided herein are methods of modifying a cell.

In some embodiments, the prime editing method comprises introducing a first PEgRNA, a second PEgRNA, and a prime editor into the cell that has the target gene. In some embodiments, the prime editing method comprises introducing into the cell that has the target gene with a prime editing composition comprising a first PEgRNA, a second PEgRNA, and a prime editor polypeptide. In some embodiments, the first PEgRNA, the second PEgRNA, and the prime editor polypeptides form complexes prior to the introduction into the cell. In some embodiments, the first PEgRNA, the second PEgRNA, and the prime editor polypeptides form complexes after the introduction into the cell. The prime editors, PEgRNAs and prime editing complexes may be introduced into the cell by any delivery approaches described herein or any delivery approach known in the art, including ribonucleoprotein (RNPs), lipid nanoparticles (LNPs), viral vectors, non-viral vectors, mRNA delivery, and physical techniques such as cell membrane disruption by a microfluidics device. The prime editors, PEgRNAs, and prime editing complexes may be introduced into the cell simultaneously or sequentially.

In some embodiments, the prime editing method comprises introducing into the cell a first PEgRNA and a second PEgRNA, or polynucleotides encoding the first PEgRNA and the second PEgRNA, and a prime editor polynucleotide encoding a prime editor polypeptide. In some embodiments, the method comprises introducing the first PEgRNA and the second PEgRNA or the polynucleotides encoding the first PEgRNA and the second PEgRNA, and the polynucleotide encoding the prime editor polypeptide into the cell simultaneously. In some embodiments, the method comprises introducing the first PEgRNA and the second PEgRNA or the polynucleotides encoding the first PEgRNA and the second PEgRNA, and the polynucleotide encoding the prime editor polypeptide into the cell sequentially. In some embodiments, the method comprises introducing the polynucleotide encoding the prime editor polypeptide into the cell before introduction of the first PEgRNA and the second PEgRNA or the polynucleotides encoding the first PEgRNA and the second PEgRNA. In some embodiments, the polynucleotide encoding the prime editor polypeptide is introduced into and expressed in the cell before introduction of the first PEgRNA and the second PEgRNA or the polynucleotides encoding the first PEgRNA and the second PEgRNA into the cell. In some embodiments, the polynucleotide encoding the prime editor polypeptide is introduced into the cell after the first PEgRNA and the second PEgRNA or the polynucleotides encoding the first PEgRNA and the second PEgRNA are introduced into the cell. The polynucleotide encoding the prime editor polypeptide, the first PEgRNA and the second PEgRNA or the polynucleotides encoding the first PEgRNA and the second PEgRNA, may be introduced into the cell by any delivery approaches described herein or any delivery approach known in the art, for example, by RNPs, LNPs, viral vectors, non-viral vectors, mRNA delivery, and physical delivery.

In some embodiments, the polynucleotide encoding the prime editor polypeptide and the polynucleotides encoding the first PEgRNA and the second PEgRNA integrate into the genome of the cell after being introduced into the cell. In some embodiments, the polynucleotide encoding the prime editor polypeptide and the polynucleotides encoding the first PEgRNA and the second PEgRNA are introduced into the cell for transient expression. Accordingly, also provided herein are cells modified by prime editing.

In some embodiments, the cell is a prokaryotic cell. In some embodiments, the cell is a eukaryotic cell. In some embodiments, the cell is a mammalian cell. In some embodiments, the cell is a non-human primate cell, bovine cell, porcine cell, rodent or mouse cell. In some embodiments, the cell is a human cell. In some embodiments, the cell is a primary cell. In some embodiments, the cell is a human primary cell. In some embodiments, the cell is a progenitor cell. In some embodiments, the cell is a human progenitor cell. In some embodiments, the cell is a muscle cell. In some embodiments, the cell is a primary muscle cell. In some embodiments, the cell is a human muscle cell. In some embodiments, the cell is a primary human muscle cell. In some embodiments, the cell is a primary human muscle cell derived from an induced human pluripotent stem cell (iPSC). In some embodiments, the cell is a neuron. In some embodiments, the cell is a neuron from basal ganglia. In some embodiments, the cell is a neuron from basal ganglia of a subject. In some embodiments, the cell is a fibroblast. In some embodiments, the cell is a human fibroblast. In some embodiments, the cell is a myogenic cell. In some embodiments, the cell is a myoblast. In some embodiments, the cell is a human myogenic cell. In some embodiments, the cell is a human myoblast. In some embodiments, the cell is a cardiac muscle cell. In some embodiments, the cell is a smooth muscle cell. In some embodiments, the cell is a myosatellite cell (also referred to as a satellite cell). In some embodiments, the cell is a human myosatellite cell. In some embodiments, the cell is a differentiated muscle cell. In some embodiments, the cell is a skeletal muscle cell. In some embodiments, the skeletal muscle cell is differentiated from an iPSC, ESC or myosatellite cell.

In some embodiments, the target gene edited by prime editing is in a chromosome of the cell. In some embodiments, the intended nucleotide edits incorporate in the chromosome of the cell and are inheritable by progeny cells. In some embodiments, the intended nucleotide edits introduced to the cell by the prime editing compositions and methods are such that the cell and progeny of the cell also include the intended nucleotide edits. In some embodiments, the cell is autologous, allogeneic, or xenogeneic to a subject. In some embodiments, the cell is from or derived from a subject. In some embodiments, the cell is from or derived from a human subject. In some embodiments, the cell is introduced back into the subject, e.g., a human subject, after incorporation of the intended nucleotide edits by prime editing.

In some embodiments, the method provided herein comprises introducing the prime editor polypeptide or the polynucleotide encoding the prime editor polypeptide, and the PEgRNAs (e.g., the first PEgRNA and the second PEgRNA) or the polynucleotides encoding the PEgRNAs, into a plurality or a population of cells that comprise the target gene. In some embodiments, the population of cells is of the same cell type. In some embodiments, the population of cells is of the same tissue or organ. In some embodiments, the population of cells is heterogeneous. In some embodiments, the population of cells is homogeneous. In some embodiments, the population of cells is from a single tissue or organ, and the cells are heterogeneous. In some embodiments, the introduction into the population of cells is ex vivo. In some embodiments, the introduction into the population of cells is in vivo, e.g., into a human subject.

In some embodiments, the target gene is in a genome of each cell of the population. In some embodiments, introduction of the prime editor polypeptide or the polynucleotide encoding the prime editor polypeptide, and the PEgRNAs (e.g., the first PEgRNA and the second PEgRNA) or the polynucleotides encoding the PEgRNAs, results in incorporation of one or more intended nucleotide edits in the target gene in at least one of the cells in the population of cells. In some embodiments, introduction of the prime editor polypeptide or the polynucleotide encoding the prime editor polypeptide, and the PEgRNAs or the polynucleotides encoding the PEgRNAs, results in incorporation of the one or more intended nucleotide edits in the target gene in a plurality of the population of cells. In some embodiments, introduction of the prime editor polypeptide or the polynucleotide encoding the prime editor polypeptide, and the PEgRNAs or the polynucleotide encoding the PEgRNAs, results in incorporation of the one or more intended nucleotide edits in the target gene in each cell of the population of cells. In some embodiments, introduction of the prime editor polypeptide or the polynucleotide encoding the prime editor polypeptide, and the PEgRNAs or the polynucleotides encoding the PEgRNAs, results in incorporation of the one or more intended nucleotide edits in the target gene in sufficient number of cells such that the disease or disorder is treated, prevented or ameliorated.

In some embodiments, editing efficiency of the prime editing compositions and method described herein can be measured by calculating the percentage of edited target genes in a population of cells introduced with the prime editing composition. The percentage of edited target genes can be assessed in any method known in the art, for example, with a next generation sequencing platform (e.g. Miseq) and suitable primers, by the percentage of edited reads over total sequenced reads. In some embodiments, the editing efficiency is determined after 1 hour, 2 hours, 6 hours, 12 hours, 24 hours, 36 hours, 48 hours, 3 days, 4 days, 5 days, 7 days, 10 days, or 14 days of exposing a target gene (e.g., a DMPK gene within the genome of a cell) to a prime editing composition. In some embodiments, the population of cells introduced with the prime editing composition is ex vivo. In some embodiments, the population of cells introduced with the prime editing composition is in vitro. In some embodiments, the population of cells introduced with the prime editing composition is in vivo.

In some embodiments, the dual prime editing compositions and methods disclosed herein have an editing efficiency of at least about 1%, at least about 5%, at least about 10%, at least about 20%, at least about 30%, at least about 40%, at least about 50%, at least about 60%, at least about 70%, at least about 80%, at least about 90%, at least about 95%, or at least about 99%. In some embodiments, the prime editing methods disclosed herein have an editing efficiency of at least 25%. In some embodiments, the prime editing methods disclosed herein have an editing efficiency of at least 30%. In some embodiments, the prime editing methods disclosed herein have an editing efficiency of at least 35%. In some embodiments, the prime editing methods disclosed herein have an editing efficiency of at least 45%. In some embodiments, the prime editing methods disclosed herein have an editing efficiency of at least 50%.

In some embodiments, the dual prime editing compositions and methods disclosed herein have an editing efficiency of at least about 1%, at least about 5%, at least about 7.5%, at least about 10%, at least about 15%, at least about 20%, at least about 30%, at least about 40%, at least about 50%, at least about 60%, at least about 70%, at least about 80%, at least about 90%, at least about 95%, or at least about 99% of editing a primary cell.

In some embodiments, the dual prime editing compositions and methods disclosed herein have an editing efficiency of at least about 5%, at least about 7.5%, at least about 10%, at least about 15%, at least about 20%, at least about 30%, at least about 40%, at least about 50%, at least about 60%, at least about 70%, at least about 80%, at least about 90%, at least about 95%, or at least about 99% of editing a muscle cell relative to a corresponding control muscle cell. In some embodiments, the muscle cell is a human muscle cell.

In some embodiments, the dual prime editing compositions and methods provided herein are capable of incorporating one or more intended nucleotide edits without generating a significant proportion of indels. The term “indel(s)”, as used herein, refers to the insertion or deletion of a nucleotide base within a polynucleotide, for example, a target gene. Such insertions or deletions can lead to frame shift mutations within a coding region of a gene. Indel frequency of editing can be calculated by methods known in the art. In some embodiments, indel frequency can be calculated based on sequence alignment such as the CRISPResso 2 algorithm as described in Clement et al., Nat. Biotechnol. 37(3): 224-226 (2019), which is incorporated herein in its entirety. In some embodiments, the methods disclosed herein can have an indel frequency of less than 20%, less than 10%, less than 9%, less than 8%, less than 7%, less than 6%, less than 5%, less than 4%, less than 3%, less than 2%, less than 1.5%, or less than 1%. In some embodiments, any number of indels is determined after at least 1 hour, at least 2 hours, at least 6 hours, at least 12 hours, at least 24 hours, at least 36 hours, at least 48 hours, at least 3 days, at least 4 days, at least 5 days, at least 7 days, at least 10 days, or at least 14 days of exposing a target gene (e.g., a DMPK gene within the genome of a cell) to a prime editing composition.

In some embodiments, the prime editing compositions provided herein are capable of incorporating one or more intended nucleotide edits efficiently without generating a significant proportion of indels. In some embodiments, the prime editing methods disclosed herein have an editing efficiency of at least about 1% and an indel frequency of less than about 5%, less than about 2.5%, less than about 1%, less than about 0.5%, or less than about 0.1% in a target cell, e.g., a human primary cell or muscle cell.

In some embodiments, the prime editing methods disclosed herein have an editing efficiency of at least about 5% and an indel frequency of less about 5%, less than about 2.5%, less than about 1%, less than about 0.5%, or less than about 0.1% in a target cell, e.g., a human primary cell or muscle cell.

In some embodiments, the prime editing methods disclosed herein have an editing efficiency of at least about 7.5% and an indel frequency of less than about 5%, less than about 2.5%, less than about 1%, less than about 0.5%, or less than about 0.1% in a target cell, e.g., a human primary cell or muscle cell.

In some embodiments, the prime editing methods disclosed herein have an editing efficiency of at least about 10% and an indel frequency of less than about 5%, less than about 2.5%, less than about 1%, less than about 0.5%, or less than about 0.1% in a target cell, e.g., a human primary cell or muscle cell.

In some embodiments, the prime editing methods disclosed herein have an editing efficiency of at least about 15% and an indel frequency of less than about 5%, less than about 2.5%, less than about 1%, less than about 0.5%, or less than about 0.1% in a target cell, e.g., a human primary cell or muscle cell.

In some embodiments, the prime editing methods disclosed herein have an editing efficiency of at least about 20% and an indel frequency of less than about 5%, less than about 2.5%, less than about 1%, less than about 0.5%, or less than about 0.1% in a target cell, e.g., a human primary cell or muscle cell.

In some embodiments, the prime editing methods disclosed herein have an editing efficiency of at least about 30% and an indel frequency of less than about 5%, less than about 2.5%, less than about 1%, less than about 0.5%, or less than about 0.1% in a target cell, e.g., a human primary cell or muscle cell.

In some embodiments, the prime editing methods disclosed herein have an editing efficiency of at least about 40% and an indel frequency of less than about 5%, less than about 2.5%, less than about 1%, less than about 0.5%, or less than about 0.1% in a target cell, e.g., a human primary cell or muscle cell.

In some embodiments, the prime editing methods disclosed herein have an editing efficiency of at least about 50% and an indel frequency of less than about 5%, less than about 2.5%, less than about 1%, less than about 0.5%, or less than about 0.1% in a target cell, e.g., a human primary cell or muscle cell.

In some embodiments, the prime editing methods disclosed herein have an editing efficiency of at least about 60% and an indel frequency of less than about 5%, less than about 2.5%, less than about 1%, less than about 0.5%, or less than about 0.1% in a target cell, e.g., a human primary cell or muscle cell.

In some embodiments, the prime editing methods disclosed herein have an editing efficiency of at least about 70% and an indel frequency of less than about 5%, less than about 2.5%, less than about 1%, less than about 0.5%, or less than about 0.1% in a target cell, e.g., a human primary cell or muscle cell.

In some embodiments, the prime editing methods disclosed herein have an editing efficiency of at least about 80% and an indel frequency of less than about 5%, less than about 2.5%, less than about 1%, less than about 0.5%, or less than about 0.1% in a target cell, e.g., a human primary cell or muscle cell.

In some embodiments, the prime editing methods disclosed herein have an editing efficiency of at least about 90% and an indel frequency of less than about 5%, less than about 2.5%, less than about 1%, less than about 0.5%, or less than about 0.1% in a target cell, e.g., a human primary cell or muscle cell.

In some embodiments, the prime editing methods disclosed herein have an editing efficiency of at least about 95% and an indel frequency of less than about 5%, less than about 2.5%, less than about 1%, less than about 0.5%, or less than about 0.1% in a target cell, e.g., a human primary cell or muscle cell.

In some embodiments, the prime editing composition described herein results in less than 50%, less than 40%, less than 30%, less than 20%, less than 19%, less than 18%, less than 17%, less than 16%, less than 15%, less than 14%, less than 13%, less than 12%, less than 11%, less than 10%, less than 9%, less than 8%, less than 7%, less than 6%, less than 5%, less than 4%, less than 3%, less than 2%, less than 1%, less than 0.9%, less than 0.8%, less than 0.7%, less than 0.6%, less than 0.5%, less than 0.4%, less than 0.3%, less than 0.2%, less than 0.1%, less than 0.09%, less than 0.08%, less than 0.07%, less than 0.06%, less than 0.05%, less than 0.04%, less than 0.03%, less than 0.02%, or less than 0.01% off-target editing in a chromosome that includes the target gene. In some embodiments, off-target editing is determined after at least 1 hour, at least 2 hours, at least 6 hours, at least 12 hours, at least 24 hours, at least 36 hours, at least 48 hours, at least 3 days, at least 4 days, at least 5 days, at least 7 days, at least 10 days, or at least 14 days of exposing a target gene (e.g., a nucleic acid within the genome of a cell) to a prime editing composition.

In some embodiments, the prime editing compositions (e.g., the PEgRNAs and prime editors as described herein) and dual prime editing methods disclosed herein can be used to edit a target DMPK gene. In some embodiments, the target DMPK gene comprises a mutation compared to a wild type DMPK gene. In some embodiments, the mutation is associated with myotonic dystrophy type 1 (DM1). In some embodiments, the target DMPK gene comprises an IND sequence that contains the mutation associated with myotonic dystrophy type 1 (DM1). In some embodiments, the PEgRNAs of the prime editing compositions direct replacement of an edited portion of a DMPK gene into the DMPK gene. In some embodiments, the mutation is associated with myotonic dystrophy (e.g., DM1). In some embodiments, the mutation is in the 3′ untranslated region of the DMPK gene. In some embodiments, the mutation is expansion of the number of CTG repeats in the 3′ untranslated region of the DMPK gene. In some embodiments, the mutation is an increased number of tri-nucleotide repeats in the array of tri-nucleotide repeats compared to a wild type DMPK gene. In some embodiments, the mutation is an array of tri-nucleotide repeats comprising the sequence (CTG)n or a complementary sequence thereof, wherein n is any integer greater than 34. In some embodiments, n is an integer greater than 50. In some embodiments, n is an integer greater than 100. In some embodiments, n is an integer greater than 200, 300, 400, 500, 600, 700, 800, 900, or 1000. In some embodiments, n is an integer greater than 1000. In some embodiments, the prime editing method comprises contacting a target DMPK gene with a prime editing composition comprising a prime editor, a first PEgRNA and a second PEgRNA. In some embodiments, contacting the target DMPK gene with the prime editing composition results in incorporation of one or more intended nucleotide edits in the target DMPK gene. In some embodiments, the incorporation is in a region of the target DMPK gene that corresponds to an IND in the DMPK gene. In some embodiments, the one or more intended nucleotide edits comprises a nucleotide substitution, an insertion, a deletion, or any combination thereof, compared to the endogenous sequence of the target DMPK gene. In some embodiments, incorporation of the one or more intended nucleotide edits results in replacement of the one or more mutations with the corresponding sequence in a wild type DMPK gene. In some embodiments, incorporation of the one more intended nucleotide edits results in correction of a mutation in the target DMPK gene. In some embodiments, the target DMPK gene comprises an IND sequence that contains the mutation. In some embodiments, contacting the target DMPK gene with the prime editing composition results in incorporation of one or more intended nucleotide edits in the target DMPK gene, which corrects the mutation in the IND in the target DMPK gene.

In some embodiments, a population of patients with mutations in the target DMPK gene may be treated with a prime editing composition (e.g., the pair of PEgRNAs and a prime editor as described herein) disclosed herein. In some embodiments, a population of patients with different distinct mutations in the target DMPK gene can be treated with a single prime editing composition comprising the same pair of PEgRNAs and a prime editor. In some embodiments, a single prime editing composition comprising the same pair of PEgRNAs and a prime editor can be used to correct one or more mutations in the target DMPK gene in a populations of patients, wherein one or more patients in the population have different mutations from one another. In some embodiments, the prime editing composition comprising the same pair of PEgRNAs and a prime editor can be used to correct two or more mutations in the target DMPK gene in a populations of patients, wherein one or more patients in the population have different mutations from one another. In some embodiments, the prime editing composition comprising the same pair of PEgRNAs and a prime editor can be used to correct 2, 3, 4, 5, 6, 7, 8, 9, 10, 15, 20, 25 or more mutations in the target DMPK gene in a populations of patients, wherein one or more patients in the population have different mutations from one another. In some embodiments, the prime editing composition comprising the same pair of PEgRNAs and a prime editor can be used to correct 30, 35, 40, 45, 50, 60 more mutations in the target DMPK gene in a populations of patients, wherein one or more patients in the population have different mutations from one another. In some embodiments, the first PEgRNA in the pair of PEgRNAs comprises a first editing template comprising a wild type sequence of the DMPK gene. In some embodiments, the second PEgRNA in the pair of PEgRNAs comprises a second editing template comprising a wild type sequence of the DMPK gene.

In some embodiments, a patient with multiple mutations in the target DMPK gene may be treated with a prime editing composition (e.g., the pair of PEgRNAs and a prime editor as described herein) disclosed herein. For example, in some embodiments, a subject may comprise two copies of the DMPK gene, each comprising one or more different mutations. In some embodiments, a patient with one or more different mutations in the target DMPK gene can be treated with a single prime editing composition comprising a pair of PEgRNAs and a prime editor.

In some embodiments, the dual prime editing composition can be used to correct all of the mutations in a portion of the DMPK gene. In some embodiments, the dual prime editing composition can be used to correct all of the mutations in the entire DMPK gene.

In some embodiments, incorporation of the one or more intended nucleotide edits results in correction of a mutation in the 3′ untranslated region of the DMPK gene. In some embodiments, the mutation is associated with myotonic dystrophy (e.g., DM1). In some embodiments, the mutation is expansion of the number of CTG repeats in the 3′ untranslated region of the DMPK gene. In some embodiments, the mutation is an increased number of tri-nucleotide repeats in the array of tri-nucleotide repeats compared to a wild type DMPK gene. In some embodiments, the mutation is an array of tri-nucleotide repeats comprising the sequence (CTG)n or a complementary sequence thereof, wherein n is any integer greater than 34. In some embodiments, n is an integer greater than 50. In some embodiments, n is an integer greater than 100. In some embodiments, n is an integer greater than 200, 300, 400, 500, 600, 700, 800, 900, or 1000. In some embodiments, n is an integer greater than 1000. In some embodiments, incorporation of the one or more intended nucleotide edits results in deletion of the CTG repeats in the 3′ untranslated region of the DMPK gene entirely. In some embodiments, incorporation of the one or more intended nucleotide edits results in reduced number of CTG repeats in the 3′ untranslated region of the DMPK gene. In some embodiments, incorporation of the one or more intended nucleotide edits results in the number of CTG repeats in the 3′ untranslated region of the DMPK gene to be less than 35. In some embodiments, incorporation of the one or more intended nucleotide edits results in the number of CTG repeats in the 3′ untranslated region of the DMPK gene to be less than 34. In some embodiments, incorporation of the one or more intended nucleotide edits results in the number of CTG repeats in the 3′ untranslated region of the DMPK gene to be less than 30, 20, or 10. In some embodiments, incorporation of the one or more intended nucleotide edits results in the number of CTG repeats in the 3′ untranslated region of the DMPK gene to be 5. In some embodiments, incorporation of the one or more intended nucleotide edits results in correction of a DMPK gene sequence and restores expression of wild type DMPK transcripts.

In some embodiments, the target DMPK gene is in a target cell. Accordingly, in one aspect provided herein is a method of editing a target cell comprising a target DMPK gene that encodes a polypeptide that comprises one or more mutations relative to a wild type DMPK gene. In some embodiments, the methods of the present disclosure comprise introducing a prime editing composition comprising a pair of PEgRNAs (i.e., first PEgRNA and a second PEgRNA), and a prime editor polypeptide into the target cell that has the target DMPK gene to edit the target DMPK gene, thereby generating an edited cell. In some embodiments, the target cell is a mammalian cell. In some embodiments, the target cell is a human cell. In some embodiments, the target cell is a primary cell. In some embodiments, the target cell is a human primary cell. In some embodiments, the target cell is a progenitor cell. In some embodiments, the target cell is a human progenitor cell. In some embodiments, the target cell is a stem cell. In some embodiments, the target cell is a human stem cell. In some embodiments, the target cell is a muscle cell. In some embodiments, the target cell is a human muscle cell. In some embodiments, the target cell is a primary human muscle cell derived from an induced human pluripotent stem cell (iPSC). In some embodiments, the cell is a neuron. In some embodiments, the cell is a neuron from basal ganglia. In some embodiments, the cell is a neuron from basal ganglia of a subject. In some embodiments, the cell is a neuron in the basal ganglia of a subject.

In some embodiments, components of a prime editing composition described herein are provided to a target cell in vitro. In some embodiments, components of a prime editing composition described herein are provided to a target cell ex vivo. In some embodiments, components of a prime editing composition described herein are provided to a target cell in vivo.

In some embodiments, incorporation of the one or more intended nucleotide edits in the target DMPK gene that comprises one or more mutations restores wild type expression and function of the DMPK protein encoded by the DMPK gene. In some embodiments, the target DMPK gene comprises an expansion of the number of CTG repeats as compared to the wild type DMPK gene prior to incorporation of the one or more intended nucleotide edits. In some embodiments, expression of a DMPK transcript with reduced number of CTG repeats compared to a DMPK transcript encoded by the endogenous DMPK gene may be measured when expressed in a target cell. In some embodiments, a change in the level of DMPK mRNA expression comprises a decrease in the amount of DMPK transcripts having 35 or more CTG repeats. In some embodiments, a change in the level of DMPK mRNA expression can comprise a fold change of, e.g., at least about 2-fold decrease, about 3-fold decrease, about 4-fold decrease, about 5-fold decrease, about 6-fold decrease, about 7-fold decrease, about 8-fold decrease, about 9-fold decrease, about 10-fold decrease, about 25-fold decrease, about 50-fold decrease, about 100-fold decrease, about 200-fold decrease, about 500-fold decrease, about 700-fold decrease, about 1000-fold decrease, about 5000-fold decrease, or about 10,000-fold decrease in the amount of DMPK transcripts having 35 or more CTG repeats.

In some embodiments, increase in expression and/or function of proteins affected by expansion of CTG repeats in the DMPK gene can be measured by a functional assay. For example, in some embodiments, function of proteins of the Muscleblind-like (MBNL) family can be measured to examine the editing efficiency of the DMPK gene.

### Methods of Treating Myotonic Dystrophy

In some embodiments, provided herein are methods for treatment of a subject diagnosed with a disease associated with or caused by one or more pathogenic mutations that can be corrected by prime editing. In some embodiments, provided herein are methods for treating myotonic dystrophy (e.g., DM1) that comprise administering to a subject a therapeutically effective amount of a prime editing composition, or a pharmaceutical composition comprising a prime editing composition as described herein.

Myotonic dystrophy is a genetic disorder that impairs muscle function. One of the main types of myotonic dystrophy is myotonic dystrophy type 1 (also referred to herein as “DM1” or “DM”). DM1 is an autosomal dominant neuromuscular disorder. Affected individuals display a wide range of symptoms including myotonia, skeletal muscle weakness and wasting, cardiac conduction abnormalities, and cataracts. DM1 is associated with mutations in the DMPK gene as set forth in the DM1 protein kinase by human genome assembly consortium Human Build 38 patch release 13 (GRCh38.p13), NCBI GenBank or RefSeq assembly accessions (GCA_000001405.28; GCF_000001405.39). The DMPK gene is located on the long arm of human chromosome 19. The DMPK gene encodes the myotonic dystrophy protein kinase (DMPK protein), which is believed to play a role in communication and impulse transmission within and between cells.

The 3′ untranslated region of the DMPK gene contains multiple copies of a cytosine-thymine-guanine (CTG) trinucleotide repeat. In most people, the number of CTG repeats in the DMPK gene ranges from about 5 to 34. DM1 is characterized by expansion of the CTG repeat numbers in the DMPK gene above a normal range. Repeat expansion is associated with disruption of local chromatin structure that may lead to dysfunction of a number of genes in the vicinity of the repeat expansion region. Repeat expansion results in increased number of repeats in the DMPK transcript, which can form stable double-stranded structures that have strong affinity for other functional proteins, e.g., proteins of the Muscleblind-like (MBNL) family, and prohibit the proteins from performing their normal functions. This causes problems for cells by trapping and disabling important proteins, consequently preventing cells in muscles and other tissues from functioning normally. The expanded repeats can also result in abnormal splicing patterns in adult tissues, which can affect expression of hundreds of genes.

The severity of myotonic dystrophy is correlated with the number of abnormally expanded CTG repeats in the DMPK gene. People with symptoms of myotonic dystrophy type 1, including muscle weakness and wasting beginning in adulthood, usually have above 100 to 1000 CTG repeats. People born with more severe, congenital form of myotonic dystrophy type 1 tend to have more than 1,000 CTG repeats in their cells. People with mild form of myotonic dystrophy type 1 symptoms usually have between 50 and 150 CTG repeats in their cells. While smaller number of CTG repeats may only relate to mild symptoms or may be asymptomatic, as the altered DMPK gene is passed from one generation to the next, the size of the CTG repeat expansion may increases in size. For example, people with 35 to 49 CTG repeats do not develop myotonic dystrophy type 1, but their children may be at risk of having the disorder if the number of CTG repeats increases.

In some embodiments, administration of the prime editing composition results in incorporation of one or more intended nucleotide edits in the target gene in the subject. In some embodiments, administration of the prime editing composition results in correction of one or more pathogenic mutations, e.g., point mutations, insertions, or deletions, associated with myotonic dystrophy (DM1) in the subject. In some embodiments, the target gene comprises a sequence, e.g., the IND sequence, that contains the pathogenic mutation. In some embodiments, administration of the prime editing composition results in incorporation of one or more intended nucleotide edits in the target gene that corrects the pathogenic mutation, or reduces the pathogenic effect of the mutation, by deleting the sequence of the IND and optionally replacing the IND sequence with one or more endogenous or exogenous sequence that has a reduced number of CTG repeats or does not comprise a CTG repeat in the target gene, thereby treating DM1 in the subject.

In some embodiments, the method provided herein comprises administering to a subject an effective amount of a prime editing composition, for example, a pair of PEgRNAs (i.e., a first PEgRNA and a second PEgRNA) and a prime editor. In some embodiments, the method comprises administering to the subject an effective amount of a prime editing composition described herein, for example, polynucleotides, vectors, or constructs that encode prime editing composition components, or RNPs, LNPs, and/or polypeptides comprising prime editing composition components. Prime editing compositions can be administered to target the DMPK gene in a subject, e.g., a human subject, suffering from, having, susceptibility to, or at risk for myotonic dystrophy (e.g., DM1). Identifying a subject in need of such treatment can be in the judgment of a subject or a health care professional and can be subjective (e.g., opinion) or objective (e.g., measurable by a test or diagnostic method). In some embodiments, the subject has myotonic dystrophy (e.g., DM1).

In some embodiments, the subject has been diagnosed with myotonic dystrophy by sequencing of a DMPK gene in the subject. In some embodiments, the subject comprises at least a copy of the DMPK gene that comprises one or more mutations compared to a wild type DMPK gene. In some embodiments, the subject comprises at least a copy of the DMPK gene that comprises a mutation in a non-coding region of the DMPK gene. In some embodiments, the subject comprises at least a copy of the DMPK gene that comprises a mutation in the 3′ untranslated region of the DMPK gene, as compared to a wild type DMPK gene. In some embodiments, the subject comprises two copies of the DMPK gene, wherein each of the two copies comprises a mutation in the 3′ untranslated region of the DMPK gene as compared to a wild type DMPK gene. In some embodiments, the mutation is increased number of CTG repeats in the 3′ untranslated region as compared to a wild type DMPK gene.

In some embodiments, the method comprises directly administering prime editing compositions provided herein to a subject. The prime editing compositions described herein can be delivered with in any form as described herein, e.g., as LNPs, RNPs, polynucleotide vectors such as viral vectors, or mRNAs. The prime editing compositions can be formulated with any pharmaceutically acceptable carrier described herein or known in the art for administering directly to a subject. Components of a prime editing composition or a pharmaceutical composition thereof may be administered to the subject simultaneously or sequentially. For example, in some embodiments, the method comprises administering a prime editing composition, or pharmaceutical composition thereof, comprising prime editor complexes that comprises (i) a prime editor fusion protein and a first PEgRNA and (ii) a prime editor fusion protein and a second PEgRNA to a subject. In some embodiments, the method comprises administering a polynucleotide or vector encoding a prime editor to a subject simultaneously with the two PEgRNAs. In some embodiments, the method comprises administering a polynucleotide or vector encoding a prime editor to a subject before administration of the two PEgRNAs. In some embodiments, the two PEgRNAs are administered simultaneously. In some embodiments, the two PEgRNAs are administered sequentially. In some embodiments, a first PEgRNA is administered with a prime editor and a second PEgRNA is administered after administration of the first PEgRNA and prime editor. In some embodiments, a first PEgRNA is administered with a prime editor and a second PEgRNA is administered before administration of the first PEgRNA and prime editor.

Suitable routes of administrating the prime editing compositions to a subject include, without limitation: topical, subcutaneous, transdermal, intradermal, intralesional, intraarticular, intraperitoneal, intravesical, transmucosal, gingival, intradental, intracochlear, transtympanic, intraorgan, epidural, intrathecal, intramuscular, intravenous, intravascular, intraosseus, periocular, intratumoral, intracerebral, and intracerebroventricular administration. In some embodiments, the compositions described are administered intraperitoneally, intravenously, or by direct injection or direct infusion. In some embodiments, the compositions described are administered by direct injection into the muscle of a subject. In some embodiments, the compositions described herein are administered to a subject by injection, by means of a catheter, by means of a suppository, or by means of an implant.

In some embodiments, the method comprises administering cells edited with a prime editing composition described herein to a subject. In some embodiments, the cells are allogeneic. In some embodiments, allogeneic cells are or have been contacted ex vivo with a prime editing composition or pharmaceutical composition thereof and are introduced into a human subject in need thereof. In some embodiments, the cells are autologous to the subject. In some embodiments, cells are removed from a subject and contacted ex vivo with a prime editing composition or pharmaceutical composition thereof and are re-introduced into the subject.

In some embodiments, cells are contacted ex vivo with one or more components of a prime editing composition. In some embodiments, the ex vivo-contacted cells are introduced into the subject, and the subject is administered in vivo with one or more components of a prime editing composition. For example, in some embodiments, cells are contacted ex vivo with a prime editor and introduced into a subject. In some embodiments, the subject is then administered with the PEgRNAs, or polynucleotides encoding the PEgRNAs.

In some embodiments, cells contacted with the prime editing composition are determined for incorporation of the one or more intended nucleotide edits in the genome before re-introduction into the subject. In some embodiments, the cells are enriched for incorporation of the one or more intended nucleotide edits in the genome before re-introduction into the subject. In some embodiments, the edited cells are primary cells. In some embodiments, the edited cells are progenitor cells. In some embodiments, the edited cells are stem cells. In some embodiments, the edited cells are muscle cells. In some embodiments, the edited cells are primary human cells. In some embodiments, the edited cells are human progenitor cells. In some embodiments, the edited cells are human stem cells. In some embodiments, the edited cells are human muscle cells. In some embodiments, the edited cells are human cardiac muscle cells, human smooth muscle cells, or human myosatellite cells). In some embodiments, the cell is a fibroblast. In some embodiments, the cell is a human fibroblast. In some embodiments, the edited cell is a myogenic cell. In some embodiments, the edited cell is a myoblast. In some embodiments, the edited cell is a human myogenic cell. In some embodiments, the edited cell is a human myoblast. In some embodiments, the cell is a neuron. In some embodiments, the cell is a neuron from basal ganglia. In some embodiments, the cell is a neuron from basal ganglia of a subject. In some embodiments, the cell is a neuron in the basal ganglia of a subject. The prime editing composition or components thereof may be introduced into a cell by any delivery approaches as described herein, including LNP administration, RNP administration, electroporation, nucleofection, transfection, viral transduction, microinjection, cell membrane disruption and diffusion, or any other approach known in the art.

The cells edited with prime editing can be introduced into the subject by any route known in the art. In some embodiments, the edited cells are administered to a subject by direct infusion. In some embodiments, the edited cells are administered to a subject by intravenous infusion. In some embodiments, the edited cells are administered to a subject as implants.

The pharmaceutical compositions, prime editing compositions, and cells, as described herein, can be administered in effective amounts. In some embodiments, the effective amount depends upon the mode of administration. In some embodiments, the effective amount depends upon the stage of the condition, the age and physical condition of the subject, the nature of concurrent therapy, if any, and like factors well-known to the medical practitioner.

The specific dose administered can be a uniform dose for each subject. Alternatively, a subject's dose can be tailored to the approximate body weight of the subject. Other factors in determining the appropriate dosage can include the disease or condition to be treated or prevented, the severity of the disease, the route of administration, and the age, sex and medical condition of the patient.

In embodiments wherein components of a prime editing composition are administered sequentially, the time between sequential administration can be at least 1 hour, at least 2 hours, at least 6 hours, at least 12 hours, at least 24 hours, at least 36 hours, at least 48 hours, at least 3 days, at least 4 days, at least 5 days, at least 7 days, at least 10 days, or at least 14 days.

In some embodiments, a method of monitoring treatment progress is provided. In some embodiments, the method includes the step of determining a level of diagnostic marker, for example, correction of a mutation in the DMPK gene, or diagnostic measurement associated with myotonic dystrophy (e.g., DM1), in a subject suffering from myotonic dystrophy symptoms and has been administered an effective amount of a prime editing composition described herein. The level of the diagnostic marker determined in the method can be compared to known levels of the marker in either healthy normal controls or in other afflicted subjects to establish the subject's disease status.

Various aspects of this disclosure provide kits comprising a prime editing composition. In one embodiment, a kit comprises a prime editing composition comprising a pair of PEgRNAs (i.e. a first PEgRNA and a second PEgRNA) and a prime editor. In one embodiment, a kit comprises a prime editing composition comprising a pair of PEgRNAs (i.e. a first PEgRNA and a second PEgRNA) and a prime editor fusion protein. In some embodiments, the kit comprises a pair of PEgRNAs (i.e. a first PEgRNA and a second PEgRNA) and a polynucleotide encoding a prime editor. In some embodiments, the kit comprises a pair of PEgRNAs (i.e. a first PEgRNA and a second PEgRNA) and a polynucleotide encoding a prime editor fusion protein. In some embodiments, the kit further provides components for delivery of the PEgRNAs and/or the prime editor. In some embodiments, the kit further provides components for delivery of the PEgRNAs and/or the prime editor fusion protein. In some embodiments, the kit further provides components for delivery of the PEgRNAs and/or the polynucleotide encoding the prime editor. In some embodiments, the kit further provides components for delivery of the PEgRNAs and/or the polynucleotide encoding the prime editor fusion protein.

In some embodiments, the kit provides instructions for using the components of the kit for prime editing. The instructions will generally include information about the use of the kit for editing nucleic acid molecules. In other embodiments, the instructions include at least one of the following: precautions; warnings; clinical studies; and/or references. The instructions may be printed directly on the container (when present), or as a label applied to the container, or as a separate sheet, pamphlet, card, or folder supplied in or with the container. In a further embodiment, a kit can comprise instructions in the form of a label or separate insert (package insert) for suitable operational parameters. In yet another embodiment, the kit can comprise one or more containers with appropriate positive and negative controls or control samples, to be used as standard(s) for detection, calibration, or normalization. The kit can further comprise a second container comprising a pharmaceutically-acceptable buffer, such as (sterile) phosphate-buffered saline, Ringer's solution, or dextrose solution. It can further include other materials desirable from a commercial and user standpoint, including other buffers, diluents, filters, needles, syringes, and package inserts with instructions for use.

### Delivery

Prime editing compositions described herein can be delivered to a cellular environment with any approach known in the art. Components of a prime editing composition can be delivered to a cell by the same mode or different modes. For example, in some embodiments, a prime editor can be delivered as a polypeptide or a polynucleotide (DNA or RNA) encoding the polypeptide. In some embodiments, a PEgRNA can be delivered directly as an RNA or as a DNA encoding the PEgRNA.

In some embodiments, a prime editing composition component is encoded by a polynucleotide, a vector, or a construct. In some embodiments, a prime editor polypeptide and PEgRNAs is encoded by a polynucleotide. In some embodiments, the polynucleotide encodes a prime editor fusion protein comprising a DNA binding domain and a DNA polymerase domain. In some embodiments, the polynucleotide encodes a DNA polymerase domain of a prime editor. In some embodiments, the polynucleotide encodes a DNA binding domain of a prime editor. In some embodiments, the polynucleotide encodes a portion of a prime editor protein, for example, a N-terminal portion of a prime editor fusion protein connected to an intein-N. In some embodiments, the polynucleotide encodes a portion of a prime editor protein, for example, a C-terminal portion of a prime editor fusion protein connected to an intein-C. In some embodiments, the polynucleotide encodes a PEgRNA. In some embodiments, the polypeptide encodes two or more components of a prime editing composition, for example, a prime editor fusion protein and a PEgRNA.

In some embodiments, the polynucleotide encoding one or more prime editing composition components that is delivered to a target cell is integrated into the genome of the cell for long-term expression, for example, by a retroviral vector. In some embodiments, the polynucleotide delivered to a target cell is expressed transiently. For example, the polynucleotide may be delivered in the form of a mRNA, or a non-integrating vector (non-integrating virus, plasmids, minicircle DNAs) for episomal expression.

In some embodiments, a polynucleotide encoding one or more prime editing system components can be operably linked to a regulatory element, e.g., a transcriptional control element, such as a promoter. In some embodiments, the polynucleotide is operably linked to multiple control elements. Depending on the expression system utilized, any of a number of suitable transcription and translation control elements, including constitutive and inducible promoters, transcription enhancer elements, transcription terminators, etc. may be used in the expression vector (e.g., U6 promoter, Hi promoter).

In some embodiments, the polynucleotide encoding one or more prime editing composition components is a part of, or is encoded by, a vector. In some embodiments, the vector is a viral vector. In some embodiments, the vector is a non-viral vector.

Non-viral vector delivery systems can include DNA plasmids, RNA (e.g., a transcript of a vector described herein), naked nucleic acid, and nucleic acid complexed with a delivery vehicle, such as a liposome. In some embodiments, the polynucleotide is provided as an RNA, e.g., a mRNA or a transcript. Any RNA of the prime editing systems, for example a guide RNA or a base editor-encoding mRNA, can be delivered in the form of RNA. In some embodiments, one or more components of the prime editing system that are RNAs is produced by direct chemical synthesis or may be transcribed in vitro from a DNA. In some embodiments, a mRNA that encodes a prime editor polypeptide is generated using in vitro transcription. Guide polynucleotides (e.g., PEgRNA) can also be transcribed using in vitro transcription from a cassette containing a T7 promoter, followed by the sequence “GG”, and guide polynucleotide sequence. In some embodiments, the prime editor encoding mRNA and/or PEgRNA(s) are synthesized in vitro using an RNA polymerase enzyme (e.g., T7 polymerase, T3 polymerase, SP6 polymerase, etc.). Once synthesized, the RNA can directly contact a target DMPK gene or can be introduced into a cell using any suitable technique for introducing nucleic acids into cells (e.g., microinjection, electroporation, transfection). In some embodiments, the prime editor-coding sequences and/or the PEgRNAs are modified to include one or more modified nucleoside e.g., using pseudo-U or 5-Methyl-C.

Methods of non-viral delivery of nucleic acids can include lipofection, nucleofection, microinjection, biolistics, virosomes, liposomes, immunoliposomes, polycation or lipid:nucleic acid conjugates, naked DNA, artificial virions, cell membrane disruption by a microfluidics device, and agent-enhanced uptake of DNA. Cationic and neutral lipids that are suitable for efficient receptor-recognition lipofection of polynucleotides can be used. Delivery can be to cells (e.g., in vitro or ex vivo administration) or target tissues (e.g., in vivo administration). The preparation of lipid:nucleic acid complexes, including targeted liposomes such as immunolipid complexes, can be used.

Viral vector delivery systems can include DNA and RNA viruses, which can have either episomal or integrated genomes after delivery to the cell. RNA or DNA viral based systems can be used to target specific cells and trafficking the viral payload to an organelle of the cell. Viral vectors can be administered directly (in vivo) or they can be used to treat cells in vitro, and the modified cells can optionally be administered (ex vivo).

In some embodiments, the viral vector is a retroviral, lentiviral, adenoviral, adeno-associated viral or herpes simplex viral vector. Retroviral vectors can include those based upon murine leukemia virus (MuLV), gibbon ape leukemia virus (GaLV), simian immunodeficiency virus (SIV), human immunodeficiency virus (HIV), and combinations thereof. In some embodiments, the retroviral vector is a lentiviral vector. In some embodiments, the retroviral vector is a gamma retroviral vector. In some embodiments, the viral vector is an adenoviral vector. In some embodiments, the viral vector is an adeno-associated virus (“AAV”) vector.

In some embodiments, polynucleotides encoding one or more prime editing composition components are packaged in a virus particle. Packaging cells can be used to form virus particles that can infect a target cell. Such cells can include 293 cells, (e.g., for packaging adenovirus), and .psi.2 (ψ²) cells or PA317 cells (e.g., for packaging retrovirus). Viral vectors can be generated by producing a cell line that packages a nucleic acid vector into a viral particle. The vectors can contain the minimal viral sequences required for packaging and subsequent integration into a host. The vectors can contain other viral sequences being replaced by an expression cassette for the polynucleotide(s) to be expressed. The missing viral functions can be supplied in trans by the packaging cell line. For example, AAV vectors can comprise ITR sequences from the AAV genome which are required for packaging and integration into the host genome.

In some embodiments, dual AAV vectors are generated by splitting a large transgene expression cassette in two separate halves (5′ and 3′ ends that encode N-terminal portion and C-terminal portion of, e.g., a prime editor polypeptide), where each half of the cassette is no more than 5 kb in length, optionally no more than 4.7 kb in length, and is packaged in a single AAV vector. In some embodiments, the full-length transgene expression cassette is reassembled upon co-infection of the same cell by both dual AAV vectors. In some embodiments, a portion or fragment of a prime editor polypeptide, e.g., a Cas9 nickase, is fused to an intein. The portion or fragment of the polypeptide can be fused to the N-terminus or the C-terminus of the intein. In some embodiments, a N-terminal portion of the polypeptide is fused to an intein-N, and a C-terminal portion of the polypeptide is separately fused to an intein-C. In some embodiments, a portion or fragment of a prime editor fusion protein is fused to an intein and fused to an AAV capsid protein. The intein, nuclease and capsid protein can be fused together in any arrangement (e.g., nuclease-intein-capsid, intein-nuclease-capsid, capsid-intein-nuclease, etc.). In some embodiments, a polynucleotide encoding a prime editor fusion protein is split in two separate halves, each encoding a portion of the prime editor fusion protein and separately fused to an intein. In some embodiments, each of the two halves of the polynucleotide is packaged in an individual AAV vector of a dual AAV vector system. In some embodiments, each of the two halves of the polynucleotide is no more than 5 kb in length, optionally no more than 4.7 kb in length. In some embodiments, the full-length prime editor fusion protein is reassembled upon co-infection of the same cell by both dual AAV vectors, expression of both halves of the prime editor fusion protein, and self-excision of the inteins.

A target cell can be transiently or non-transiently transfected with one or more vectors described herein. A cell can be transfected as it naturally occurs in a subject. A cell can be taken or derived from a subject and transfected. A cell can be derived from cells taken from a subject, such as a cell line. In some embodiments, a cell transfected with one or more vectors described herein can be used to establish a new cell line comprising one or more vector-derived sequences. In some embodiments, a cell transiently transfected with the compositions of the disclosure (such as by transient transfection of one or more vectors, or transfection with RNA), and modified through the activity of a prime editor, can be used to establish a new cell line comprising cells containing the modification but lacking any other exogenous sequence. Any suitable vector compatible with the host cell can be used with the methods of the disclosure. Non-limiting examples of vectors include pXT1, pSG5, pSVK3, pBPV, pMSG, and pSVLSV40.

In some embodiments, a prime editor protein can be provided to cells as a polypeptide. In some embodiments, the prime editor protein is fused to a polypeptide domain that increases solubility of the protein. In some embodiments, the prime editor protein is formulated to improve solubility of the protein.

In some embodiment, a prime editor polypeptide is fused to a polypeptide permeant domain to promote uptake by the cell. In some embodiments, the permeant domain is a peptide, a peptidomimetic, or a non-peptide carrier. For example, a permeant peptide may be derived from the third alpha helix of Drosophila melanogaster transcription factor Antennapaedia, referred to as penetratin, which comprises the amino acid sequence RQIKIWFQNRRMKWKK (SEQ ID NO: 3778). As another example, the permeant peptide can comprise the HIV-1 tat basic region amino acid sequence, which may include, for example, amino acids 49-57 of naturally-occurring tat protein. Other permeant domains can include poly-arginine motifs, for example, the region of amino acids 34-56 of HIV-1 rev protein, nona-arginine, and octa-arginine. The nona-arginine (R9) sequence can be used. The site at which the fusion can be made may be selected in order to optimize the biological activity, secretion or binding characteristics of the polypeptide.

In some embodiments, a prime editor polypeptide is produced in vitro or by host cells, and it may be further processed by unfolding, e.g., heat denaturation, DTT reduction, etc. and may be further refolded. In some embodiments, a prime editor polypeptide is prepared by in vitro synthesis. Various commercial synthetic apparatuses can be used. By using synthesizers, naturally occurring amino acids can be substituted with unnatural amino acids. In some embodiments, a prime editor polypeptide is isolated and purified in accordance with recombinant synthesis methods, for example, by expression in a host cell and the lysate purified using HPLC, exclusion chromatography, gel electrophoresis, affinity chromatography, or other purification technique.

In some embodiments, a prime editing composition, for example, prime editor polypeptide components and PEgRNA(s) are introduced to a target cell by nanoparticles. In some embodiments, the prime editor polypeptide components and the PEgRNA form a complex in the nanoparticle. Any suitable nanoparticle design can be used to deliver genome editing system components or nucleic acids encoding such components. In some embodiments, the nanoparticle is inorganic. In some embodiments, the nanoparticle is organic. In some embodiments, a prime editing composition is delivered to a target cell, e.g., a muscle cell, in an organic nanoparticle, e.g., a lipid nanoparticle (LNP) or polymer nanoparticle.

In some embodiments, LNPs are formulated from cationic, anionic, neutral lipids, or combinations thereof. In some embodiments, neutral lipids, such as the fusogenic phospholipid DOPE or the membrane component cholesterol, are included to enhance transfection activity and nanoparticle stability. In some embodiments, LNPs are formulated with hydrophobic lipids, hydrophilic lipids, or combinations thereof. Lipids may be formulated in a wide range of molar ratios to produce an LNP. Any lipid or combination of lipids that are known in the art can be used to produce an LNP. Exemplary lipids used to produce LNPs are provided in Table 48 below.

In some embodiments, components of a prime editing composition form a complex prior to delivery to a target cell. For example, a prime editor fusion protein and a PEgRNA can form a complex prior to delivery to the target cell. In some embodiments, a prime editing polypeptide (e.g., a prime editor fusion protein) and a guide polynucleotide (e.g., a PEgRNA) form a ribonucleoprotein (RNP) for delivery to a target cell. In some embodiments, the RNP comprises a prime editor fusion protein in complex with a PEgRNA. RNPs may be delivered to cells using known methods, such as electroporation, nucleofection, or cationic lipid-mediated methods, or any other approaches known in the art. In some embodiments, delivery of a prime editing composition or complex to the target cell does not require the delivery of foreign DNA into the cell. In some embodiments, the RNP comprising the prime editing complex is degraded over time in the target cell. Exemplary lipids for use in nanoparticle formulations and/or gene transfer are shown in Table 48 below.

TABLE 48
Exemplary lipids for nanoparticle formulation or gene transfer

| Lipid | Abbreviation | Feature |
|---|---|---|
| 1,2-Dioleoyl-sn-glycero-3-phosphatidylcholine | DOPC | Helper |
| 1,2-Dioleoyl-sn-glycero-3- | DOPE | Helper |
| phosphatidylethanolamine | | |
| Cholesterol | | Helper |
| N41-(2,3-Dioleyloxy)prophyliN,N,N- | DOTMA | Cationic |
| trimethylammonium chloride | | |
| 1,2-Dioleoyloxy-3-trimethylammonium-propane | DOGS | Cationic |
| Dioctadecylamidoglycylspermine | | |
| N-(3-Aminopropyl)-N,N-dimethyl-2,3- | GAP-DLRIE | Cationic |
| bis(dodecyloxy)-1-propanaminium bromide | | |
| Cetyltrimethylammonium bromide | CTAB | Cationic |
| 6-Lauroxyhexyl omithinate | LHON | Cationic |
| 1-(2,3-Dioleoyloxypropyl)-2,4,6- | 2Oc | Cationic |
| trimethylpyridinium | | |
| 2,3-Dioleyloxy-N-P(spenninecarboxamido- | DOSPA | Cationic |
| ethylJ-N,Ndimethyl-1- | | |
| propanatninium trifluoroacetate | | |
| 1,2-Dioleyl-3-trimethylamtnonium-propane | DOPA | Cationic |
| N-(2-Hydroxyethyl)-N,N-dimethyl-2,3- | MDRIE | Cationic |
| bis(tetradecyloxy)-1-propanaminium bromide | | |
| Dimyristooxypropyl dimethyl hydroxyethyl | DMRI | Cationic |
| ammonium bromide | | |
| 3β-[N-(N′,N′-Dimethylaminoethane)- | DC-Chol | Cationic |
| carbamoyl]cholesterol | | |
| Bis-guanidium-tren-cholesterol | BGTC | Cationic |
| 1,3-Diodeoxy-2-(6-carboxy-spermyl)- | DOSPER | Cationic |
| propylamide | | |
| Dimethyloctadecylammonium bromide | DDAB | Cationic |
| Dioctadecylamidoglicylspermidin | DSL | Cationic |
| rac-[(2,3-Dioctadecyloxypropyl)(2- | CLIP-1 | Cationic |
| hydroxyethyl)]-dimethylammonium chloride | | |
| rac-[2(2,3-Dihexadecyloxypropyloxymethyloxy) | CLIP-6 | Cationic |
| ethyl]trimethylammoniun bromide | | |
| Ethyldimyristoylphosphatidylcholine | EDMPC | Cationic |
| 1,2-Distearyloxy-N,N-dimethyl-3-aminopropane | DSDMA | Cationic |
| 1,2-Dimyristoyl-trimethylammonium propane | DMTAP | Cationic |
| O,O′-Dimyristyl-N-lysyl aspartate | DMKE | Cationic |
| 1,2-Distearoyl-sn-glycero-3-ethylphospho- | DSEPC | Cationic |
| choline | | |
| N-Palmitoyl D-erythro-sphingosyl | CCS | Cationic |
| carbamoyl-spenmine | | |
| N-t-Butyl-N0-tetradecyl-3- | diC14- | Cationic |
| tetradecylaminopropionamidine | amidine | |
| Octadecenolyoxy[ethyl-2-heptadecenyl-3 | DOTIM | Cationic |
| hydroxyethyl] imidazolinium chloride | | |
| N1-Cholesteryloxycarbonyl-3,7-diazanonane- | CDAN | Cationic |
| 1,9-diamine | | |
| 2-(3-Bis(3-amino-propyl)-amino]propylamino)- | RPR209120 | Cationic |
| Nditetradecylcarbamoylme-ethyl-acetamide | | |
| 1,2-dilinoleyloxy-3-dimethylaminopropane | DLinDMA | Cationic |
| 2,2-dilinoleyl-4-dimethylaminoethyl- | DLin-KC2- | Cationic |
| [1,3]-dioxolane | DMA | |
| dilinoleyl-methyl-4-dimethylaminobutyrate | DLin-MC3- | Cationic |
| | DMA | |

Exemplary polymers for use in nanoparticle formulations and/or gene transfer are shown in Table 49 below.

TABLE 49
Exemplary polymers for nanoparticle formulation or gene transfer

| Polymer | Abbreviation |
|---|---|
| Poly(ethylene)glycol | PEG |
| Polyethylenimine | PEI |
| Dithiobis (succinimidylpropionate) | DSP |
| Dimethyl-3,3′-dithiobispropionimidate | DTBP |
| Poly(ethylene imine)biscarbamate | PEIC |
| Poly(L-lysine) | PLL |
| Histidine modified PLL | |
| Poly(N-vinylpyrrolidone) | PVP |
| Poly(propylenimine) | PPI |
| Poly(amidoamine) | PAMAM |
| Poly(amidoethylenimine) | SS_PAEI |
| Triethylenetetramine | TETA |
| Poly(β-aminoester) | |
| Poly(4-hydroxy-L-proline ester) | PHP |
| Poly(allylamine) | |
| Poly(α-[4-aminobutyl]-L-glycolic acid) | PAGA |
| Poly(D,L-lactic-co-glycolic acid) | PLGA |
| Poly(N-ethyl-4-vinylpyridinium bromide) | |
| Poly(phosphazene)s | PPZ |
| Poly(phosphoester)s | PPE |
| Poly(phosphoramidate)s | PPA |
| Poly(N-2-hydroxypropylmethacrylamide) | pHPMA |
| Poly (2-(dimethylamino)ethyl methacrylate) | pDMAEMA |
| Poly(2-aminoethyl propylene phosphate) | PPE-EA |
| Chitosan | |
| Galactosylated chitosan | |
| N-dodacylated chitosam | |
| Histone | |
| Collagen | |
| Dextran-spermine | D-SPM |

Exemplary delivery methods for polynucleotides encoding prime editing composition components are shown in Table 50 below.

TABLE 50
Exemplary polynucleotide delivery methods

| | | Delivery | | | |
|---|---|---|---|---|---|
| | | into Non- | | | Type of |
| | | Dividing | Duration of | Genome | Molecule |
| Delivery | Vector/Mode | Cells | Expression | Integration | Delivered |
| Physical | (e.g., | YES | Transient | NO | Nucleic |
| | electroporation, | | | | Acids and |
| | particle gun, | | | | Proteins |
| | Calcium | | | | |
| | phosphate | | | | |
| | transfection) | | | | |
| Viral | Retrovirus | NO | Stable | YES | RNA |
| | Lentivirus | YES | Stable | YES/NO with | RNA |
| | | | | modification | |
| | Adenovirus | YES | Transient | NO | DNA |
| | Adeno-Associated | YES | Stable | NO | DNA |
| | Virus (AAV) | | | | |
| | Vaccinia Virus | YES | Very | NO | DNA |
| | | | Transient | | |
| | Herpes | YES | Stable | NO | DNA |
| | Simplex Virus | | | | |
| Non-Viral | Cationic | YES | Transient | Depends on | Nucleic |
| | | | | what is | acids and |
| | | | | delivered | Proteins |
| | Polymeric | YES | Transient | NO | Nucleic |
| | Nanoparticles | | | | Acids |
| Biological | Attenuated | YES | Transient | NO | Nucleic |
| | Bacteria | | | | Acids |
| Non-Viral | Engineered | YES | Transient | NO | Nucleic |
| Delivery | Bacteriophages | | | | Acids |
| Vehicles | Mammalian | YES | Transient | NO | Nucleic |
| | Virus-like | | | | Acids |
| | Particles | | | | |
| | Biological | YES | Transient | NO | Nucleic |
| | liposomes: | | | | Acids |
| | Erythrocyte | | | | |
| | Ghosts and | | | | |
| | Exosomes | | | | |

The prime editing compositions of the disclosure, whether introduced as polynucleotides or polypeptides, can be provided to the cells for about 30 minutes to about 24 hours, e.g., 1 hour, 1.5 hours, 2 hours, 2.5 hours, 3 hours, 3.5 hours 4 hours, 5 hours, 6 hours, 7 hours, 8 hours, 12 hours, 16 hours, 18 hours, 20 hours, or any other period from about 30 minutes to about 24 hours, which can be repeated with a frequency of about every day to about every 4 days, e.g., every 1.5 days, every 2 days, every 3 days, or any other frequency from about every day to about every four days. The compositions may be provided to the subject cells one or more times, e.g., one time, twice, three times, or more than three times, and the cells allowed to incubate with the agent(s) for some amount of time following each contacting event e.g., 16-24 hours. In cases in which two or more different prime editing system components, e.g., two different polynucleotide constructs are provided to the cell (e.g., different components of the same prime editing system, or two different guide nucleic acids that are complementary to different sequences within the same or different target genes), the compositions may be delivered simultaneously (e.g., as two polypeptides and/or nucleic acids). Alternatively, they may be provided sequentially, e.g., one composition being provided first, followed by a second composition.

The prime editing compositions and pharmaceutical compositions of the disclosure, whether introduced as polynucleotides or polypeptides, can be administered to subjects in need thereof for about 30 minutes to about 24 hours, e.g., 1 hour, 1.5 hours, 2 hours, 2.5 hours, 3 hours, 3.5 hours 4 hours, 5 hours, 6 hours, 7 hours, 8 hours, 12 hours, 16 hours, 18 hours, 20 hours, or any other period from about 30 minutes to about 24 hours, which can be repeated with a frequency of about every day to about every 4 days, e.g., every 1.5 days, every 2 days, every 3 days, or any other frequency from about every day to about every four days. The compositions may be provided to the subject one or more times, e.g., one time, twice, three times, or more than three times. In cases in which two or more different prime editing system components, e.g., two different polynucleotide constructs are administered to the subject (e.g., different components of the same prime editing system, or two different guide nucleic acids that are complementary to different sequences within the same or different target genes), the compositions may be administered simultaneously (e.g., as two polypeptides and/or nucleic acids). Alternatively, they may be provided sequentially, e.g., one composition being provided first, followed by a second composition.

The following embodiments are within the scope of the present disclosure. Furthermore, the disclosure encompasses all variations, combinations, and permutations of these embodiments in which one or more limitations, elements, clauses, and descriptive terms from one or more of the listed embodiments is introduced into another listed embodiment in this section. For example, any listed embodiment that is dependent on another embodiment can be modified to include one or more limitations found in any other listed embodiment in this section that is dependent on the same base embodiment.

Embodiment 1: In some embodiments, a prime editing composition of the present disclosure comprises: (A) a first prime editing guide RNA (PEgRNA) or one or more polynucleotides encoding the first PEgRNA, and (B) a second PEgRNA or one or more polynucleotides encoding the second PEgRNA; wherein the first PEgRNA comprises: (i) a first spacer that is complementary to a first search target sequence on a first strand of a DMPK gene; (ii) a first gRNA core capable of binding to a Cas9 protein; and (iii) a first extension arm comprising a first editing template and a first primer binding site (PBS); wherein the first spacer comprises at its 3′ end nucleotides 5-20 of a sequence selected from the group consisting of SEQ ID NOs: 1, 20, 39, 58, 77, 484, 518, 536, 554, 572, 590, 912, 929, 947, 965, 983, 1001, 1019, and 1341, and wherein the first PBS comprises at its 5′ end a sequence that is the reverse complement of nucleotides 15-17 of the selected sequence; wherein the second PEgRNA comprises: (i) a second spacer that is complementary to a second search target sequence on a second strand of the DMPK gene complementary to the first strand; (ii) a second gRNA core capable of binding to a Cas9 protein; and (iii) a second extension arm comprising a second editing template and a second PBS; wherein the second spacer comprises at its 3′ end nucleotides 5-20 of a sequence selected from the group consisting of SEQ ID NOs: 1359, 1381, 1403, 1503, 1525, 1864, 1882, 1900, 1918, 1936, 2263, 2281, 2299, 2317, 2335, 2353, 2673, and wherein the second PBS comprises at its 5′ end a sequence that is the reverse complement of nucleotides 15-17 of the selected sequence; and wherein (a) the first editing template comprises a region of complementarity to the second editing template; (b) the first editing template comprises nucleotides 8-17 of the selected sequence for the second spacer, and the second editing template comprises nucleotides 8-17 of the selected sequence for the first spacer; or (c) the first editing template comprises nucleotides 8-17 of the selected sequence for the second spacer, and a region of complementarity to the second editing template, and the second editing template comprises nucleotides 8-17 of the selected sequence for the first spacer, and a region of complementarity to the first editing template.

Embodiment 2: The prime editing composition of Embodiment 1, wherein the selected sequence for the first spacer is SEQ ID NOs: 77, 484, 536, 590, or 1019.

Embodiment 3: The prime editing composition of Embodiment 1 or 2, wherein the selected sequence for the second spacer is SEQ ID NO: 1525, 1900, 1936, 2263, 2281, 2299, 2353, or 2673.

Embodiment 4: The prime editing composition of any one of Embodiments 1-3, wherein the selected sequence for the first spacer is SEQ ID NO: 77, 536, or 1019.

Embodiment 5: The prime editing composition of any one of Embodiments 1-4, wherein the selected sequence for the second spacer is SEQ ID NO: 1900, 1936, or 2673.

Embodiment 6: The prime editing composition of any one of Embodiments 1-5, wherein the first spacer and/or the second spacer is from 16 to 22 nucleotides in length.

Embodiment 7: The prime editing composition of any one of Embodiments 1-6, wherein the first spacer and/or the second spacer comprises at its 3′ the selected sequence.

Embodiment 8: The prime editing composition of Embodiment 7, wherein the first spacer and/or the second spacer is 20 nucleotides in length and comprises the selected sequence.

Embodiment 9: The prime editing composition of any one of Embodiments 1-8, wherein the first gRNA core and the second gRNA core comprise the same sequence.

Embodiment 10: The prime editing composition of Embodiment 9, wherein the first gRNA core and the second gRNA core each comprises SEQ ID NO: 3641.

Embodiment 11: The prime editing composition of any one of Embodiments 1-10, wherein the first spacer, the first gRNA core, the first editing template, and the first PBS form a contiguous sequence in a single molecule.

Embodiment 12: The prime editing composition of Embodiment 11, wherein the first PEgRNA comprises from 5′ to 3′ the first spacer, the first gRNA core, the first editing template, and the first PBS.

Embodiment 13: The prime editing composition of any one of Embodiments 1-12, wherein the second spacer, the second gRNA core, the second editing template, and the second PBS form a contiguous sequence in a single molecule.

Embodiment 14: The prime editing composition of Embodiment 13, wherein the second pegRNA comprises from 5′ to 3′ the second spacer, the second gRNA core, the second editing template, and the second PBS.

Embodiment 15: The prime editing composition of any one of Embodiments 1-14, where in the first PBS is at least 8 nucleotides in length and comprises at its 5′ end a sequence that is the reverse complement of nucleotides 10-17 of the selected sequence for the first spacer.

Embodiment 16: The prime editing composition of Embodiment 15, wherein the first PBS is 8-17 nucleotides in length and comprises at its 5′ end a sequence that is the reverse complement of nucleotides 10-17, 9-17, 8-17, 7-17, 6-17, 5-17, 4-17, 3-17, 2-17, or 1-17 of the selected sequence for the first spacer.

Embodiment 17: The prime editing composition of Embodiment 16, wherein the first PBS is 8-14 nucleotides in length.

Embodiment 18: The prime editing composition of Embodiment 16, wherein the first PBS is 8-12 nucleotides in length.

Embodiment 19: The prime editing composition of Embodiment 16, wherein the first PBS is 8-10 nucleotides in length.

Embodiment 20: The prime editing composition of Embodiment 16, wherein the first PBS is 8-9 nucleotides in length.

Embodiment 21: The prime editing composition of Embodiment 16, wherein the first PBS is 10 nucleotides in length.

Embodiment 22: The prime editing composition of any one of Embodiments 1-21, where in the second PBS is at least 8 nucleotides in length and comprises at its 5′ end a sequence that is the reverse complement of nucleotides 10-17 of the selected sequence for the second spacer.

Embodiment 23: The prime editing composition of Embodiment 22, wherein the second PBS is 8-17 nucleotides in length and comprises at its 5′ end a sequence that is the reverse complement of nucleotides 10-17, 9-17, 8-17, 7-17, 6-17, 5-17, 4-17, 3-17, 2-17, or 1-17 of the selected sequence for the second spacer.

Embodiment 24: The prime editing composition of Embodiment 23, wherein the second PBS is 8-14 nucleotides in length.

Embodiment 25: The prime editing composition of Embodiment 23, wherein the second PBS is 8-12 nucleotides in length.

Embodiment 26: The prime editing composition of Embodiment 23, wherein the second PBS is 8-10 nucleotides in length.

Embodiment 27: The prime editing composition of Embodiment 23, wherein the second PBS is 8-9 nucleotides in length.

Embodiment 28: The prime editing composition of Embodiment 23, wherein the second PBS is 10 nucleotides in length.

Embodiment 29: The prime editing composition of any one of Embodiments 1-28, wherein the first editing template comprises a region of complementarity to the second editing template.

Embodiment 30: The prime editing composition of Embodiment 29, wherein the region of complementarity is about 20 to about 80 nucleotides in length.

In some embodiments, a prime editing composition of Embodiment 29 comprises a region of complementarity that is about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, or about 80 nucleotides in length.

In some embodiments, a prime editing composition of Embodiment 29 comprises a region of complementarity that is between 20 and 80 nucleotides; between 21 and 80 nucleotides; between 22 and 80 nucleotides; between 23 and 80 nucleotides; between 24 and 80 nucleotides; between 25 and 80 nucleotides; between 26 and 80 nucleotides; between 27 and 80 nucleotides; between 28 and 80 nucleotides; between 29 and 80 nucleotides; between 30 and 80 nucleotides; between 31 and 80 nucleotides; between 32 and 80 nucleotides; between 33 and 80 nucleotides; between 34 and 80 nucleotides; between 35 and 80 nucleotides; between 36 and 80 nucleotides; between 37 and 80 nucleotides; between 38 and 80 nucleotides; between 39 and 80 nucleotides; between 40 and 80 nucleotides; between 41 and 80 nucleotides; between 42 and 80 nucleotides; between 43 and 80 nucleotides; between 44 and 80 nucleotides; between 45 and 80 nucleotides; between 46 and 80 nucleotides; between 47 and 80 nucleotides; between 48 and 80 nucleotides; between 49 and 80 nucleotides; between 50 and 80 nucleotides; between 51 and 80 nucleotides; between 52 and 80 nucleotides; between 53 and 80 nucleotides; between 54 and 80 nucleotides; between 55 and 80 nucleotides; between 56 and 80 nucleotides; between 57 and 80 nucleotides; between 58 and 80 nucleotides; between 59 and 80 nucleotides; between 60 and 80 nucleotides; between 61 and 80 nucleotides; between 62 and 80 nucleotides; between 63 and 80 nucleotides; between 64 and 80 nucleotides; between 65 and 80 nucleotides; between 66 and 80 nucleotides; between 67 and 80 nucleotides; between 68 and 80 nucleotides; between 69 and 80 nucleotides; between 70 and 80 nucleotides; between 71 and 80 nucleotides; between 72 and 80 nucleotides; between 73 and 80 nucleotides; between 74 and 80 nucleotides; between 75 and 80 nucleotides; between 76 and 80 nucleotides; between 77 and 80 nucleotides; between 78 and 80 nucleotides; between 20 and 80 nucleotides; between 20 and 79 nucleotides; between 20 and 78 nucleotides; between 20 and 77 nucleotides; between 20 and 76 nucleotides; between 20 and 75 nucleotides; between 20 and 74 nucleotides; between 20 and 73 nucleotides; between 20 and 72 nucleotides; between 20 and 71 nucleotides; between 20 and 70 nucleotides; between 20 and 69 nucleotides; between 20 and 68 nucleotides; between 20 and 67 nucleotides; between 20 and 66 nucleotides; between 20 and 65 nucleotides; between 20 and 64 nucleotides; between 20 and 63 nucleotides; between 20 and 62 nucleotides; between 20 and 61 nucleotides; between 20 and 60 nucleotides; between 20 and 59 nucleotides; between 20 and 58 nucleotides; between 20 and 57 nucleotides; between 20 and 56 nucleotides; between 20 and 55 nucleotides; between 20 and 54 nucleotides; between 20 and 53 nucleotides; between 20 and 52 nucleotides; between 20 and 51 nucleotides; between 20 and 50 nucleotides; between 20 and 49 nucleotides; between 20 and 48 nucleotides; between 20 and 47 nucleotides; between 20 and 46 nucleotides; between 20 and 45 nucleotides; between 20 and 44 nucleotides; between 20 and 43 nucleotides; between 20 and 42 nucleotides; between 20 and 41 nucleotides; between 20 and 40 nucleotides; between 20 and 39 nucleotides; between 20 and 38 nucleotides; between 20 and 37 nucleotides; between 20 and 36 nucleotides; between 20 and 35 nucleotides; between 20 and 34 nucleotides; between 20 and 33 nucleotides; between 20 and 32 nucleotides; between 20 and 31 nucleotides; between 20 and 30 nucleotides; between 20 and 29 nucleotides; between 20 and 28 nucleotides; between 20 and 27 nucleotides; between 20 and 26 nucleotides; between 20 and 25 nucleotides; between 20 and 24 nucleotides; between 20 and 23 nucleotides; or between 20 and 22 nucleotides in length.

In some embodiments, a prime editing composition of Embodiment 29 comprises a region of complementarity that is at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, or at least 80 nucleotides in length.

In some embodiments, a prime editing composition of Embodiment 29 comprises a region of complementarity that is no more than 20, no more than 21, no more than 22, no more than 23, no more than 24, no more than 25, no more than 26, no more than 27, no more than 28, no more than 29, no more than 30, no more than 31, no more than 32, no more than 33, no more than 34, no more than 35, no more than 36, no more than 37, no more than 38, no more than 39, no more than 40, no more than 41, no more than 42, no more than 43, no more than 44, no more than 45, no more than 46, no more than 47, no more than 48, no more than 49, no more than 50, no more than 51, no more than 52, no more than 53, no more than 54, no more than 55, no more than 56, no more than 57, no more than 58, no more than 59, no more than 60, no more than 61, no more than 62, no more than 63, no more than 64, no more than 65, no more than 66, no more than 67, no more than 68, no more than 69, no more than 70, no more than 71, no more than 72, no more than 73, no more than 74, no more than 75, no more than 76, no more than 77, no more than 78, no more than 79, or no more than 80 nucleotides in length.

Embodiment 31: The prime editing composition of Embodiment 29, wherein the region of complementarity is about 20 to about 40 nucleotides in length.

Embodiment 32. The prime editing composition of Embodiment 29, wherein the region of complementarity is 23-38 nucleotides in length.

Embodiment 32a: The prime editing composition of Embodiment 29, wherein the region of complementarity is about 10-19 nucleotides in length.

Embodiment 32b: The prime editing composition of Embodiment 29, wherein the region of complementarity is 10-19 nucleotides in length.

Embodiment 32c: The prime editing composition of Embodiment 29, wherein the region of complementarity is about 20-29 nucleotides in length.

Embodiment 32d: The prime editing composition of Embodiment 29, wherein the region of complementarity is 20-29 nucleotides in length.

Embodiment 32e: The prime editing composition of Embodiment 29, wherein the region of complementarity is about 30-40 nucleotides in length.

Embodiment 32f: The prime editing composition of Embodiment, wherein the region of complementarity is 30-40 nucleotides in length.

Embodiment 33: The prime editing composition of Embodiment 29, wherein the GC content of the region of complementarity is about 40% to about 80%.

Embodiment 34: The prime editing composition of Embodiment 29, wherein the GC content of the region of complementarity is about 50% to about 80%.

Embodiment 35: The prime editing composition of Embodiment 29, wherein the GC content of the region of complementarity is about 60% to about 80%.

Embodiment 36: The prime editing composition of Embodiment 29, wherein the GC content of the region of complementarity is at least 63%.

Embodiment 37: The prime editing composition of Embodiment 29, wherein the GC content of the region of complementarity is 63%-79%.

Embodiment 37a: The prime editing composition of Embodiment 29, wherein the GC content of the region of complementarity is no more than about 45%.

Embodiment 37b: The prime editing composition of Embodiment 29, wherein the GC content of the region of complementarity is no more than 45%.

Embodiment 37c: The prime editing composition of Embodiment 29, wherein the GC content of the region of complementarity is about 45%-60%.

Embodiment 37d: The prime editing composition of Embodiment 29, wherein the GC content of the region of complementarity is 45%-60%.

Embodiment 37e: The prime editing composition of Embodiment 29, wherein the GC content of the region of complementarity is about 60%-75%.

Embodiment 37e: The prime editing composition of Embodiment 29, wherein the GC content of the region of complementarity is 60%-75%.

Embodiment 37f: The prime editing composition of Embodiment 29, wherein the GC content of the region of complementarity is at least about 75%.

Embodiment 37g: The prime editing composition of Embodiment 29, wherein the GC content of the region of complementarity is at least 75%.

Embodiment 38: The prime editing composition of any one of Embodiments 29-37, wherein the first editing template comprises SEQ ID NO: 2691.

Embodiment 39: The prime editing composition of any one of Embodiments 29-38, wherein the second editing template comprises SEQ ID NO: 3075.

Embodiment 40: The prime editing composition of any one of Embodiments 29-37, where in the first editing template comprises a sequence selected from the group consisting of SEQ ID NOs: 2692-2736.

Embodiment 41: The prime editing composition of any one of Embodiments 29-37 and 40, wherein the second editing template comprises a sequence selected from the group consisting of SEQ ID NOs: 3076-3120.

Embodiment 41a: The prime editing composition of any one of Embodiments 29-37, wherein the first editing template comprises a sequence selected from the group consisting of SEQ ID NOs: 2692-2700.

Embodiment 41b: The prime editing composition of any one of Embodiments 29-37 and 41a, wherein the second editing template comprises a sequence selected from the group consisting of SEQ ID NOs: 3076-3084.

Embodiment 41c: The prime editing composition of any one of Embodiments 29-37, wherein the first editing template comprises a sequence selected from the group consisting of SEQ ID NOs: 2701-2709.

Embodiment 41d: The prime editing composition of any one of Embodiments 29-37 and 41c, wherein the second editing template comprises a sequence selected from the group consisting of SEQ ID NOs: 3085-3093.

Embodiment 41e: The prime editing composition of any one of Embodiments 29-37, wherein the first editing template comprises a sequence selected from the group consisting of SEQ ID NOs: 2710-2718.

Embodiment 41f: The prime editing composition of any one of Embodiments 29-37 and 41e, wherein the second editing template comprises a sequence selected from the group consisting of SEQ ID NOs: 3094-3102.

Embodiment 41g: The prime editing composition of any one of Embodiments 29-37, wherein the first editing template comprises a sequence selected from the group consisting of SEQ ID NOs: 2719-2727.

Embodiment 41h: The prime editing composition of any one of Embodiments 29-37 and 41g, wherein the second editing template comprises a sequence selected from the group consisting of SEQ ID NOs: 3103-3111.

Embodiment 41i: The prime editing composition of any one of Embodiments 29-37, wherein the first editing template comprises a sequence selected from the group consisting of SEQ ID NOs: 2728-2736.

Embodiment 41j: The prime editing composition of any one of Embodiments 29-37 and 41i, wherein the second editing template comprises a sequence selected from the group consisting of SEQ ID NOs: 3112-3120.

Embodiment 42: The prime editing composition of any one of Embodiments 29-37, wherein the first editing template comprises a sequence selected from the group consisting of SEQ ID NOs: 2737-2769.

Embodiment 43: The prime editing composition of any one of Embodiments 29-37 and 42, wherein the second editing template comprises a sequence selected from the group consisting of SEQ ID NOs: 3121-3153.

Embodiment 43a: The prime editing composition of any one of Embodiments 29-37, wherein the first editing template comprises a sequence selected from the group consisting of SEQ ID NOs: 2737-2745 and 2769.

Embodiment 43b: The prime editing composition of any one of Embodiments 29-37 and 43a, wherein the second editing template comprises a sequence selected from the group consisting of SEQ ID NOs: 3121-3129 and 3153.

Embodiment 43c: The prime editing composition of any one of Embodiments 29-37, wherein the first editing template comprises a sequence selected from the group consisting of SEQ ID NOs: 2746-2749.

Embodiment 43d: The prime editing composition of any one of Embodiments 29-37 and 43c, wherein the second editing template comprises a sequence selected from the group consisting of SEQ ID NOs: 3130-3133.

Embodiment 43e: The prime editing composition of any one of Embodiments 29-37, wherein the first editing template comprises a sequence selected from the group consisting of SEQ ID NOs: 2750-2758.

Embodiment 43f: The prime editing composition of any one of Embodiments 29-37 and 43e, wherein the second editing template comprises a sequence selected from the group consisting of SEQ ID NOs: 3134-3142.

Embodiment 43g: The prime editing composition of any one of Embodiments 29-37, wherein the first editing template comprises a sequence selected from the group consisting of SEQ ID NOs: 2759-2768.

Embodiment 43h: The prime editing composition of any one of Embodiments 29-37 and 43g, wherein the second editing template comprises a sequence selected from the group consisting of SEQ ID NOs: 3143-3152.

Embodiment 43i: The prime editing composition of any one of Embodiments 29-37g, wherein the first editing template comprises a sequence selected from the group consisting of SEQ ID NOs: 2770-3074.

Embodiment 43j: The prime editing composition of any one of Embodiments 29-37g, wherein the second editing template comprises a sequence selected from the group consisting of SEQ ID NOs: 3154-3458.

Embodiment 43k: The prime editing composition of Embodiment 43i or 43j, wherein the region of complementarity is 10 to 19 nt in length and has a GC content of at least about 75%.

Embodiment 43l: The prime editing composition of Embodiment 43k, wherein the first editing template comprises SEQ ID NOs: 2796 or 2804.

Embodiment 43m: The prime editing composition of Embodiment 43k or 43l, wherein the second editing template comprises SEQ ID NO: 3180 or 3188.

Embodiment 43n: The prime editing composition of Embodiment 43k, wherein the first editing template comprises a sequence selected from the group consisting of SEQ ID NOs: 2895, 3008, 3011, 2847, 2859, and 2864.

Embodiment 43o: The prime editing composition of Embodiment 43k or 43n, wherein the second editing template comprises a sequence selected from the group consisting of SEQ ID NOs: 3279, 3392, 3395, 3231, 3243, and 3248.

Embodiment 43p: The prime editing composition of Embodiment 43i or 43j, wherein the region of complementarity is 10 to 19 nt in length and has a GC content of about 45%-60%.

Embodiment 43q: The prime editing composition of Embodiment 43p, wherein the first editing template comprises SEQ ID NOs: 2792 or 3014.

Embodiment 43r: The prime editing composition of Embodiment 43p or 43q, wherein the second editing template comprises SEQ ID NOs: 3176 or 3398:

Embodiment 43s. The prime editing composition of Embodiment 43p, wherein the first editing template comprises a sequence selected from the group consisting of SEQ ID NOs: 2819, 2786, and 2835.

Embodiment 43t. The prime editing composition of Embodiment 43p or 43s, wherein the second editing template comprises a sequence selected from the group consisting of SEQ ID NOs: 3203, 3170, and 3219.

Embodiment 43u: The prime editing composition of Embodiment 43i or 43j, wherein the region of complementarity is 10 to 19 nt in length and has a GC content of less than about 45%.

Embodiment 43v: The prime editing composition of Embodiment 43u, wherein the first editing template comprises SEQ ID NO: 2770 or 2803.

Embodiment 43w: The prime editing composition of Embodiment 43u or 43v, wherein the second editing template comprises SEQ ID NO: 3154 or 3187.

Embodiment 43x: The prime editing composition of Embodiment 43u, wherein the first editing template comprises a sequence selected from the group consisting of SEQ ID NOs: 2812, 2929, and 2797.

Embodiment 43v: The prime editing composition of Embodiment 43u or 43x, wherein the second editing template comprises a sequence selected from the group consisting of SEQ ID NOs: 3196, 3313, and 3181.

Embodiment 43z: The prime editing composition of Embodiment 43i or 43j, wherein the region of complementarity is 10 to 19 nt in length and has a GC content of about 60%-75%.

Embodiment 43aa: The prime editing composition of Embodiment 43z, wherein the first editing template comprises SEQ ID NOs: 2781 or 2977.

Embodiment 43bb: The prime editing composition of Embodiment 43z or 43aa, wherein the second editing template comprises a SEQ ID NOs: 3165 or 3361.

Embodiment 43cc: The prime editing composition of Embodiment 43z, wherein the first editing template comprises a sequence selected from the group consisting of SEQ ID NOs: 2802, 2823, 2939, 2831, and 2909.

Embodiment 43dd: The prime editing composition of Embodiment 43z or 43cc, wherein the second editing template comprises a sequence selected from the group consisting of SEQ ID NOs: 3186, 3207, 3323, 3215, and 3293.

Embodiment 43ee: The prime editing composition of Embodiment 43i or 43j, wherein the region of complementarity is 20-30 nt in length and has a GC content of at least about 75%.

Embodiment 43ff: The prime editing composition of Embodiment 43ee, wherein the first editing template comprises SEQ ID NO: 2851

Embodiment 43gg: The prime editing composition of Embodiment 43ee or 43ff, wherein the second editing template comprises SEQ ID NO: 3235.

Embodiment 43hh: The prime editing composition of Embodiment 43i or 43j, wherein the region of complementarity is 20-30 nt in length and has a GC content of about 45%-60%.

Embodiment 43ii: The prime editing composition of Embodiment 43hh, wherein the first editing template comprises SEQ ID NOs: 2924 or 3066.

Embodiment 43jj: The prime editing composition of Embodiment 43hh or 43ii, wherein the second editing template comprises SEQ ID NOs: 3308 or 3450.

Embodiment 43kk: The prime editing composition of Embodiment 43hh, wherein the first editing template comprises a sequence selected from the group consisting of SEQ ID NOs: 2925, 2995, 2950, and 2978.

Embodiment 43ll: The prime editing composition of Embodiment 43hh or 43kk, wherein the second editing template comprises a sequence selected from the group consisting of SEQ ID NOs: 3309, 3379, 3334, and 3362.

Embodiment 43 mm: The prime editing composition of Embodiment 43i or 43j, wherein the region of complementarity is 20 to 30 nt in length and has a GC content of less than about 45%.

Embodiment 43nn: The prime editing composition of Embodiment 43 mm, wherein the first editing template comprises SEQ ID NOs: 2777 or 3032.

Embodiment 43oo: The prime editing composition of Embodiment 43 mm or 43nn, wherein the second editing template comprises SEQ ID NO:3161 or 3416.

Embodiment 43pp: The prime editing composition of Embodiment 43 mm, wherein the first editing template comprises a sequence selected from the group consisting of SEQ ID NOs: 3040, 3072, and 2773.

Embodiment 43qq: The prime editing composition of Embodiment 43 mm or 43pp, wherein the second editing template comprises a sequence selected from the group consisting of SEQ ID NOs: 3424, 3456, and 3157.

Embodiment 43rr: The prime editing composition of Embodiment 43i or 43j, wherein the region of complementarity is 20 to 30 nt in length and has a GC content of about 60%-75%.

Embodiment 43ss: The prime editing composition of Embodiment 43rr, wherein the first editing template comprises SEQ ID No 2980 or 3039.

Embodiment 43tt: The prime editing composition of Embodiment 43rr or 43ss, wherein the second editing template comprises a sequence selected from the group consisting of SEQ ID NOs: 3364 or 3423.

Embodiment 43uu: The prime editing composition of Embodiment 43rr, wherein the first editing template comprises SEQ ID NOs: 2997 or 2849.

Embodiment 43vv: The prime editing composition of Embodiment 43rr or 43uu, wherein the second editing template SEQ ID NOs: 3381, or 3233.

Embodiment 43ww: The prime editing composition of Embodiment 43i or 43j, wherein the region of complementarity is at least 30 nt in length and has a GC content of about 45%-60%, optionally wherein the region of complementarity is 30 to 40 nt in length.

Embodiment 43xx: The prime editing composition of Embodiment 43ww, wherein the first editing template comprises SEQ ID NO: 3013.

Embodiment 43vv: The prime editing composition of Embodiment 43ww or 43xx, wherein the second editing template comprises a sequence selected from the group consisting of SEQ ID NO: 3397.

Embodiment 43zz: The prime editing composition of Embodiment 43yy, wherein the first editing template comprises SEQ ID NO: 2820 or 2976.

Embodiment 43aaa: The prime editing composition of Embodiment 43yy or 43zz, wherein the second editing template comprises SEQ ID NO: 3204 or 3360.

Embodiment 43bbb: The prime editing composition of Embodiment 43i or 43j, wherein the region of complementarity is at least 30 nt in length and has a GC content of less than about 45%, optionally wherein the region of complementarity is 30 to 40 nt in length.

Embodiment 43ccc: The prime editing composition of Embodiment 43bbb, wherein the first editing template comprises SEQ ID NO: 2943 or 3020.

Embodiment 43ddd: The prime editing composition of Embodiment 43bbb or 43ccc, wherein the second editing template comprises a sequence selected from the group consisting of SEQ ID NOs: 3327 or 3404.

Embodiment 43eee: The prime editing composition of Embodiment 43bbb, wherein the first editing template comprises SEQ ID NO: 2778 or 3061.

Embodiment 43fff: The prime editing composition of Embodiment 43bbb or 43eee, wherein the second editing template comprises SEQ ID NOs: 3162 or 3445.

Embodiment 44: The prime editing composition of any one of Embodiments 29-43, wherein: (i) the first spacer comprises SEQ ID NO: 77, and the first PBS comprises SEQ ID No: 85; (ii) the first spacer comprises SEQ ID NO: 536, and the first PBS comprises SEQ ID No: 544; or (iii) the first spacer comprises SEQ ID NO: 1019, and the first PBS comprises SEQ ID No:1027; and wherein: (i) the second spacer comprises SEQ ID NO: 1900, and the second PBS comprises SEQ ID NO: 1908; (ii) the second spacer comprises SEQ ID NO: 1936, and the second PBS comprises SEQ ID NO: 1944; or (iii) the second spacer comprises SEQ ID NO: 2673, and the second PBS comprises SEQ ID NO: 2681.

Embodiment 45: The prime editing composition of any one of Embodiments 29-43, wherein the first spacer comprises SEQ ID No: 77, and the first PBS is 8-10 nucleotides in length and comprises SEQ ID NOs: 87, 86, or 85.

Embodiment 46: The prime editing composition of Embodiment 45, wherein the second spacer comprises SEQ ID NO: 1936, and the second PBS is 8-10 nucleotides in length and comprises SEQ ID NOs: 1946, 1945, or 1944.

Embodiment 47: The prime editing composition of any one of Embodiments 44-46, wherein the first PEgRNA comprises a sequence selected from the group consisting of SEQ ID NOs: 93-102, 552, 553, 1035, and 1036, and wherein the second PEgRNA comprises a sequence selected from the group consisting of SEQ ID NOs: 1952-1961, 1916, 1917, 2689, and 2690.

Embodiment 48: The prime editing composition of any one of Embodiments 44-47, wherein the first PEgRNA comprises a sequence selected from the group consisting of SEQ ID NOs: 94, 552, and 1035; and wherein the second PEgRNA comprises a sequence selected from the group consisting of 1952, 1916, and 2689.

Embodiment 49: The prime editing composition of any one of Embodiments 29-48, wherein the first editing template and the second editing template have the same length and are perfectly complementary to each other.

Embodiment 49a: The prime editing composition of Embodiment 29, wherein the first spacer comprises at its 3′ end nucleotides 5-20 of SEQ ID NO: 77, and wherein the first editing template comprises a sequence selected from the group consisting of SEQ ID NOs: 2771, 2800, 2811, 2775, 2778, 2820, 2990, 2776, 2779, 2780, 2786, 2799, 2802, 2812, 2819, 2937, 2996, 3010, 3041, 3044, 3058, 2813, 3068, 3070, and 3072.

Embodiment 49b: The prime editing composition of Embodiment 29, wherein the first spacer comprises at its 3′ end nucleotides 5-20 of SEQ ID NO: 1019, and wherein the first editing template comprises a sequence selected from the group consisting of SEQ ID NOs: 2795, 2949, 2816, 2775, 2778, 2820, 2990, 2776, 2779, 2780, 2786, 2799, 2802, 2812, 2819, 2937, 2996, 3010, 3041, 2813, 3068, 3070, and 3072.

Embodiment 49c: The prime editing composition of Embodiment 29, wherein the first spacer comprises at its 3′ end nucleotides 5-20 of SEQ ID NO: 590, and wherein the first editing template comprises a sequence selected from the group consisting of SEQ ID NOs: 2774, 2834, 2896, 2940, 2962, 3009, 30302816, 2776, 2779, 2780, 2786, 2799, 2802, 2812, 2819, 2937, 2996, 3010, 3041, 3044, 3058, and 3072.

Embodiment 49d: The prime editing composition of any one of Embodiments 29 and 49a-49c, wherein the second spacer comprises at its 3′ end nucleotides 5-20 of SEQ ID NO: 1525, and wherein the second editing template comprises a sequence selected from the group consisting of SEQ ID NOs: 3195, 3158, 3414, 3393, 3162, 3204, 3164, 3170, 3203, 3380, 3425, 3442, 3197, 3454, 3159, 3186, 3196, 3456, 3280, 3160, and 3163.

Embodiment 49e: The prime editing composition of any one of Embodiments 29 and 49a-49c, wherein the second spacer comprises at its 3′ end nucleotides 5-20 of SEQ ID NO: 1936, and wherein the second editing template comprises a sequence selected from the group consisting of SEQ ID NOs: 3184, 3218, 3346, 3321, 3428, 3452, 3393, 3162, 3204, 3164, 3170, 3203, 3380, 3425, 3442, 3197, 3454, 3159, 3186, 3196, and 3456.

Embodiment 49f: The prime editing composition of any one of Embodiments 29 and 49a-49c, wherein the second spacer comprises at its 3′ end nucleotides 5-20 of SEQ ID NO: 2353, and wherein the second editing template comprises a sequence selected from the group consisting of SEQ ID NOs: 3155, 3324, 3179, 3333, 3200, 3374, 3183, 3394, 3452, 3393, 3162, 3204, 3164, 3170, 3203, 3380, 3425, 3442, 3197, 3454, 3280, 3160, and 3163.

Embodiment 49k: The prime editing composition of any one of Embodiments 29-49f, wherein the first editing template and the second editing template have the same length and are perfectly complementary to each other.

Embodiment 50: The prime editing composition of any one of Embodiments 1-28, wherein the first editing template comprises at its 3′ end nucleotides 8-17 of the selected sequence for the second spacer, and wherein the second editing template comprises at its 3′ end nucleotides 8-17 of the selected sequence of the first spacer.

Embodiment 51: The prime editing composition of Embodiment 50, wherein the first editing template comprises at its 3′ end nucleotides 1-17, 2-17, 3-17, 4-17, 5-17, 6-17, 7-17, or 8-17 of the selected sequence for the second spacer, and/or wherein the second editing template comprises at its 3′ end nucleotides 1-17, 2-17, 3-17, 4-17, 5-17, 6-17, 7-17, or 8-17 of the selected sequence for the first spacer.

Embodiment 52: The prime editing composition of Embodiment 50, wherein the first editing template comprises at its 3′ end nucleotides 3-17 of the selected sequence for the second spacer, and wherein the second editing template comprises at its 3′ end nucleotides 3-17 of the selected sequence of the first spacer.

Embodiment 53: The prime editing composition of Embodiment 52, wherein the first editing template comprises a region of complementarity to a sequence on the second strand of the DMPK gene that is directly 3′ to nucleotide 3 of the second search target sequence, wherein the region of complementarity is about 20, about 25, about 30 about 35, about 40, about 45, about 50 nucleotides in length.

In some embodiments, a prime editing composition of Embodiment 52 comprises a first editing template that comprises a region of complementarity to a sequence on the second strand of the DMPK gene that is directly 3′ to nucleotide 3 of the second search target sequence, wherein the region of complementarity is about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, or about 50 nucleotides in length.

In some embodiments, a prime editing composition of Embodiment 52 comprises a first editing template that comprises a region of complementarity to a sequence on the second strand of the DMPK gene that is directly 3′ to nucleotide 3 of the second search target sequence, wherein the region of complementarity is between 20 and 50 nucleotides; between 21 and 50 nucleotides; between 22 and 50 nucleotides; between 23 and 50 nucleotides; between 24 and 50 nucleotides; between 25 and 50 nucleotides; between 26 and 50 nucleotides; between 27 and 50 nucleotides; between 28 and 50 nucleotides; between 29 and 50 nucleotides; between 30 and 50 nucleotides; between 31 and 50 nucleotides; between 32 and 50 nucleotides; between 33 and 50 nucleotides; between 34 and 50 nucleotides; between 35 and 50 nucleotides; between 36 and 50 nucleotides; between 37 and 50 nucleotides; between 38 and 50 nucleotides; between 39 and 50 nucleotides; between 40 and 50 nucleotides; between 41 and 50 nucleotides; between 42 and 50 nucleotides; between 43 and 50 nucleotides; between 44 and 50 nucleotides; between 45 and 50 nucleotides; between 46 and 50 nucleotides; between 47 and 50 nucleotides; between 48 and 50 nucleotides; between 20 and 50 nucleotides; between 20 and 49 nucleotides; between 20 and 48 nucleotides; between 20 and 47 nucleotides; between 20 and 46 nucleotides; between 20 and 45 nucleotides; between 20 and 44 nucleotides; between 20 and 43 nucleotides; between 20 and 42 nucleotides; between 20 and 41 nucleotides; between 20 and 40 nucleotides; between 20 and 39 nucleotides; between 20 and 38 nucleotides; between 20 and 37 nucleotides; between 20 and 36 nucleotides; between 20 and 35 nucleotides; between 20 and 34 nucleotides; between 20 and 33 nucleotides; between 20 and 32 nucleotides; between 20 and 31 nucleotides; between 20 and 30 nucleotides; between 20 and 29 nucleotides; between 20 and 28 nucleotides; between 20 and 27 nucleotides; between 20 and 26 nucleotides; between 20 and 25 nucleotides; between 20 and 24 nucleotides; between 20 and 23 nucleotides; or between 20 and 22 nucleotides in length.

In some embodiments, a prime editing composition of Embodiment 52 comprises a first editing template that comprises a region of complementarity to a sequence on the second strand of the DMPK gene that is directly 3′ to nucleotide 3 of the second search target sequence, wherein the region of complementarity is at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, or at least 50 nucleotides in length.

In some embodiments, a prime editing composition of Embodiment 52 comprises a first editing template that comprises a region of complementarity to a sequence on the second strand of the DMPK gene that is directly 3′ to nucleotide 3 of the second search target sequence, wherein the region of complementarity is no more than 20, no more than 21, no more than 22, no more than 23, no more than 24, no more than 25, no more than 26, no more than 27, no more than 28, no more than 29, no more than 30, no more than 31, no more than 32, no more than 33, no more than 34, no more than 35, no more than 36, no more than 37, no more than 38, no more than 39, no more than 40, no more than 41, no more than 42, no more than 43, no more than 44, no more than 45, no more than 46, no more than 47, no more than 48, no more than 49, or no more than 50 nucleotides in length.

Embodiment 54: The prime editing composition of Embodiment 53, wherein the region of complementarity is about 20-30 nucleotides in length.

Embodiment 55: The prime editing composition of Embodiment 52, wherein the second editing template comprises a region of complementarity to a sequence on the first strand of the DMPK gene that is directly 3′ to nucleotide 3 of the first search target sequence, wherein the region of complementarity is about 20, about 25, about 30, about 35, about 40, about 45, or about 50 nucleotides in length.

Embodiment 56: The prime editing composition of Embodiment 55, wherein the region of complementarity is about 20-30 nucleotides in length.

Embodiment 57: The prime editing composition of any one of Embodiments 50-56, wherein the first editing template comprises a sequence selected from the group consisting of SEQ ID NOs: 3459-3461, wherein the second spacer comprises at its 3′ end nucleotides 5-20 of SEQ ID NO: 1525, optionally wherein the second spacer comprises at its 3 end SEQ ID NO: 1525.

Embodiment 57a. The prime editing composition of any one of Embodiments 50-56, wherein the first editing template comprises a sequence selected from the group consisting of SEQ ID NOs: 3467-3471, wherein the second spacer comprises at its 3′ end nucleotides 5-20 of SEQ ID NO: 1525, optionally wherein the second spacer comprises at its 3 end SEQ ID NO: 1525.

Embodiment 57b. The prime editing composition of any one of Embodiments 50-56, wherein the first editing template comprises a sequence selected from the group consisting of SEQ ID NOs: 3462-3466, and wherein the second spacer comprises at its 3′ end nucleotides 5-20 of SEQ ID NO: 2353, optionally wherein the second spacer comprises at its 3 end SEQ ID NO: 2353.

Embodiment 57c. The prime editing composition of any one of Embodiments 50-56, wherein the first editing template comprises a sequence selected from the group consisting of SEQ ID NOs: 3472-3476, wherein the second spacer comprises at its 3′ end nucleotides 5-20 of SEQ ID NO: 1936, optionally wherein the second spacer comprises at its 3 end SEQ ID NO: 1936.

Embodiment 57d. The prime editing composition of any one of Embodiments 50-56, wherein the first editing template comprises nucleotides 31-40, 30-40, 29-40, 28-40, 27-40, 26-40, 25-40, 24-40, 23-40, 22-40, 21-40, 20-40, 19-40, 18-40, 17-40, 16-40, 15-40, 14-40, 13-40, 12-40, 11-40, 10-40, 9-40, 8-40, 7-40, 6-40, 5-40, 4-40, 3-40, 2-40, or 1-40 of SEQ ID NO: 3647, wherein the second spacer comprises at its 3′ end nucleotides 5-20 of SEQ ID NO:1900, optionally wherein the second spacer comprises at its 3′ end SEQ ID NO: 1900.

Embodiment 57e. The prime editing composition of any one of Embodiments 50-56, wherein the first editing template comprises nucleotides 31-40, 30-40, 29-40, 28-40, 27-40, 26-40, 25-40, 24-40, 23-40, 22-40, 21-40, 20-40, 19-40, 18-40, 17-40, 16-40, 15-40, 14-40, 13-40, 12-40, 11-40, 10-40, 9-40, 8-40, 7-40, 6-40, 5-40, 4-40, 3-40, 2-40, or 1-40 of SEQ ID NO: 3648, wherein the second spacer comprises at its 3′ end nucleotides 5-20 of SEQ ID NO: 1918, optionally wherein the second spacer comprises at its 3′ end SEQ ID NO: 1918.

Embodiment 57f. The prime editing composition of any one of Embodiments 50-56, wherein the first editing template comprises nucleotides 31-40, 30-40, 29-40, 28-40, 27-40, 26-40, 25-40, 24-40, 23-40, 22-40, 21-40, 20-40, 19-40, 18-40, 17-40, 16-40, 15-40, 14-40, 13-40, 12-40, 11-40, 10-40, 9-40, 8-40, 7-40, 6-40, 5-40, 4-40, 3-40, 2-40, or 1-40 of SEQ ID NO: 3649, wherein the second spacer comprises at its 3′ end nucleotides 5-20 of SEQ ID NO: 1403, optionally wherein the second spacer comprises at its 3′ end SEQ ID NO: 1403.

Embodiment 57g. The prime editing composition of any one of Embodiments 50-56, wherein the first editing template comprises nucleotides 31-40, 30-40, 29-40, 28-40, 27-40, 26-40, 25-40, 24-40, 23-40, 22-40, 21-40, 20-40, 19-40, 18-40, 17-40, 16-40, 15-40, 14-40, 13-40, 12-40, 11-40, 10-40, 9-40, 8-40, 7-40, 6-40, 5-40, 4-40, 3-40, 2-40, or 1-40 of SEQ ID NO: 3650, wherein the second spacer comprises at its 3′ end nucleotides 5-20 of SEQ ID NO: 1936, optionally wherein the second spacer comprises at its 3′ end SEQ ID NO: 1936.

Embodiment 57h. The prime editing composition of any one of Embodiments 50-56, wherein the first editing template comprises nucleotides 31-40, 30-40, 29-40, 28-40, 27-40, 26-40, 25-40, 24-40, 23-40, 22-40, 21-40, 20-40, 19-40, 18-40, 17-40, 16-40, 15-40, 14-40, 13-40, 12-40, 11-40, 10-40, 9-40, 8-40, 7-40, 6-40, 5-40, 4-40, 3-40, 2-40, or 1-40 of SEQ ID NO: 3651, wherein the second spacer comprises at its 3′ end nucleotides 5-20 of SEQ ID NO: 2263, optionally wherein the second spacer comprises at its 3′ end SEQ ID NO: 2263.

Embodiment 57i. The prime editing composition of any one of Embodiments 50-56, wherein the first editing template comprises nucleotides 31-40, 30-40, 29-40, 28-40, 27-40, 26-40, 25-40, 24-40, 23-40, 22-40, 21-40, 20-40, 19-40, 18-40, 17-40, 16-40, 15-40, 14-40, 13-40, 12-40, 11-40, 10-40, 9-40, 8-40, 7-40, 6-40, 5-40, 4-40, 3-40, 2-40, or 1-40 of SEQ ID NO: 3652, wherein the second spacer comprises at its 3′ end nucleotides 5-20 of SEQ ID NO: 2353, optionally wherein the second spacer comprises at its 3′ end SEQ ID NO: 2353.

Embodiment 57j. The prime editing composition of any one of Embodiments 50-56, wherein the first editing template comprises nucleotides 31-40, 30-40, 29-40, 28-40, 27-40, 26-40, 25-40, 24-40, 23-40, 22-40, 21-40, 20-40, 19-40, 18-40, 17-40, 16-40, 15-40, 14-40, 13-40, 12-40, 11-40, 10-40, 9-40, 8-40, 7-40, 6-40, 5-40, 4-40, 3-40, 2-40, or 1-40 of SEQ ID NO: 3653, wherein the second spacer comprises at its 3′ end nucleotides 5-20 of SEQ ID NO: 1503, optionally wherein the second spacer comprises at its 3′ end SEQ ID NO: 1503.

Embodiment 57k. The prime editing composition of any one of Embodiments 50-56, wherein the first editing template comprises nucleotides 31-40, 30-40, 29-40, 28-40, 27-40, 26-40, 25-40, 24-40, 23-40, 22-40, 21-40, 20-40, 19-40, 18-40, 17-40, 16-40, 15-40, 14-40, 13-40, 12-40, 11-40, 10-40, 9-40, 8-40, 7-40, 6-40, 5-40, 4-40, 3-40, 2-40, or 1-40 of SEQ ID NO: 3654, wherein the second spacer comprises at its 3′ end nucleotides 5-20 of SEQ ID NO: 2281, optionally wherein the second spacer comprises at its 3′ end SEQ ID NO: 2281.

Embodiment 57l. The prime editing composition of any one of Embodiments 50-56, wherein the first editing template comprises nucleotides 31-40, 30-40, 29-40, 28-40, 27-40, 26-40, 25-40, 24-40, 23-40, 22-40, 21-40, 20-40, 19-40, 18-40, 17-40, 16-40, 15-40, 14-40, 13-40, 12-40, 11-40, 10-40, 9-40, 8-40, 7-40, 6-40, 5-40, 4-40, 3-40, 2-40, or 1-40 of SEQ ID NO: 3655, wherein the second spacer comprises at its 3′ end nucleotides 5-20 of SEQ ID NO: 2673, optionally wherein the second spacer comprises at its 3′ end SEQ ID NO: 2673.

Embodiment 57m. The prime editing composition of any one of Embodiments 50-56, wherein the first editing template comprises nucleotides 31-40, 30-40, 29-40, 28-40, 27-40, 26-40, 25-40, 24-40, 23-40, 22-40, 21-40, 20-40, 19-40, 18-40, 17-40, 16-40, 15-40, 14-40, 13-40, 12-40, 11-40, 10-40, 9-40, 8-40, 7-40, 6-40, 5-40, 4-40, 3-40, 2-40, or 1-40 of SEQ ID NO: 3656, wherein the second spacer comprises at its 3′ end nucleotides 5-20 of SEQ ID NO: 1525, optionally wherein the second spacer comprises at its 3′ end SEQ ID NO: 1525.

Embodiment 57n. The prime editing composition of any one of Embodiments 50-56, wherein the first editing template comprises nucleotides 31-40, 30-40, 29-40, 28-40, 27-40, 26-40, 25-40, 24-40, 23-40, 22-40, 21-40, 20-40, 19-40, 18-40, 17-40, 16-40, 15-40, 14-40, 13-40, 12-40, 11-40, 10-40, 9-40, 8-40, 7-40, 6-40, 5-40, 4-40, 3-40, 2-40, or 1-40 of SEQ ID NO: 3657, wherein the second spacer comprises at its 3′ end nucleotides 5-20 of SEQ ID NO: 2299, optionally wherein the second spacer comprises at its 3′ end SEQ ID NO: 2299.

Embodiment 57o. The prime editing composition of any one of Embodiments 50-56, wherein the first editing template comprises nucleotides 31-40, 30-40, 29-40, 28-40, 27-40, 26-40, 25-40, 24-40, 23-40, 22-40, 21-40, 20-40, 19-40, 18-40, 17-40, 16-40, 15-40, 14-40, 13-40, 12-40, 11-40, 10-40, 9-40, 8-40, 7-40, 6-40, 5-40, 4-40, 3-40, 2-40, or 1-40 of SEQ ID NO: 3658, wherein the second spacer comprises at its 3′ end nucleotides 5-20 of SEQ ID NO: 2317, optionally wherein the second spacer comprises at its 3′ end SEQ ID NO: 2317.

Embodiment 57p. The prime editing composition of any one of Embodiments 50-56, wherein the first editing template comprises nucleotides 31-40, 30-40, 29-40, 28-40, 27-40, 26-40, 25-40, 24-40, 23-40, 22-40, 21-40, 20-40, 19-40, 18-40, 17-40, 16-40, 15-40, 14-40, 13-40, 12-40, 11-40, 10-40, 9-40, 8-40, 7-40, 6-40, 5-40, 4-40, 3-40, 2-40, or 1-40 of SEQ ID NO: 3659, wherein the second spacer comprises at its 3′ end nucleotides 5-20 of SEQ ID NO: 1864, optionally wherein the second spacer comprises at its 3′ end SEQ ID NO: 1864.

Embodiment 57g. The prime editing composition of any one of Embodiments 50-56, wherein the first editing template comprises nucleotides 31-40, 30-40, 29-40, 28-40, 27-40, 26-40, 25-40, 24-40, 23-40, 22-40, 21-40, 20-40, 19-40, 18-40, 17-40, 16-40, 15-40, 14-40, 13-40, 12-40, 11-40, 10-40, 9-40, 8-40, 7-40, 6-40, 5-40, 4-40, 3-40, 2-40, or 1-40 of SEQ ID NO: 3660, wherein the second spacer comprises at its 3′ end nucleotides 5-20 of SEQ ID NO: 1359, optionally wherein the second spacer comprises at its 3′ end SEQ ID NO: 1359.

Embodiment 57r. The prime editing composition of any one of Embodiments 50-56, wherein the first editing template comprises nucleotides 31-40, 30-40, 29-40, 28-40, 27-40, 26-40, 25-40, 24-40, 23-40, 22-40, 21-40, 20-40, 19-40, 18-40, 17-40, 16-40, 15-40, 14-40, 13-40, 12-40, 11-40, 10-40, 9-40, 8-40, 7-40, 6-40, 5-40, 4-40, 3-40, 2-40, or 1-40 of SEQ ID NO: 3661, wherein the second spacer comprises at its 3′ end nucleotides 5-20 of SEQ ID NO: 2335, optionally wherein the second spacer comprises at its 3′ end SEQ ID NO: 2335.

Embodiment 57s. The prime editing composition of any one of Embodiments 50-56, wherein the first editing template comprises nucleotides 31-40, 30-40, 29-40, 28-40, 27-40, 26-40, 25-40, 24-40, 23-40, 22-40, 21-40, 20-40, 19-40, 18-40, 17-40, 16-40, 15-40, 14-40, 13-40, 12-40, 11-40, 10-40, 9-40, 8-40, 7-40, 6-40, 5-40, 4-40, 3-40, 2-40, or 1-40 of SEQ ID NO: 3662, wherein the second spacer comprises at its 3′ end nucleotides 5-20 of SEQ ID NO: 1882, optionally wherein the second spacer comprises at its 3′ end SEQ ID NO: 1882.

Embodiment 57t. The prime editing composition of any one of Embodiments 50-56, wherein the first editing template comprises nucleotides 31-40, 30-40, 29-40, 28-40, 27-40, 26-40, 25-40, 24-40, 23-40, 22-40, 21-40, 20-40, 19-40, 18-40, 17-40, 16-40, 15-40, 14-40, 13-40, 12-40, 11-40, 10-40, 9-40, 8-40, 7-40, 6-40, 5-40, 4-40, 3-40, 2-40, or 1-40 of SEQ ID NO: 3663, wherein the second spacer comprises at its 3′ end nucleotides 5-20 of SEQ ID NO: 1381, optionally wherein the second spacer comprises at its 3′ end SEQ ID NO: 1381.

Embodiment 58: The prime editing composition of any one of Embodiments 50-57, wherein the second editing template comprises a sequence selected from the group consisting of SEQ ID NOs: 3477-3479, wherein the first spacer comprises at its 3′ end nucleotides 5-20 of SEQ ID NO: 484, optionally wherein the first spacer comprises at its 3′ end SEQ ID NO: 484.

Embodiment 58a. The prime editing composition of any one of Embodiments 50-58, wherein the second editing template comprises a sequence selected from the group consisting of SEQ ID NOs: 3480-3484, wherein the first spacer comprises at its 3′ end nucleotides 5-20 of SEQ ID NO: 77, optionally wherein the first spacer comprises at its 3 end SEQ ID NO: 77.

Embodiment 58b. The prime editing composition of any one of Embodiments 50-58, wherein the second editing template comprises a sequence selected from the group consisting of SEQ ID NOs: 3485-3489, wherein the first spacer comprises at its 3′ end nucleotides 5-20 of SEQ ID NO: 590, optionally wherein the first spacer comprises at its 3 end SEQ ID NO: 590.

Embodiment 58c. The prime editing composition of any one of Embodiments 50-58, wherein the second editing template comprises a sequence selected from the group consisting of SEQ ID NOs: 3490-3494, wherein the first spacer comprises at its 3′ end nucleotides 5-20 of SEQ ID NO: 1019, optionally wherein the first spacer comprises at its 3 end SEQ ID NO: 1019.

Embodiment 58d. The prime editing composition of any one of Embodiments 50-58, wherein the second editing template comprises nucleotides 31-40, 30-40, 29-40, 28-40, 27-40, 26-40, 25-40, 24-40, 23-40, 22-40, 21-40, 20-40, 19-40, 18-40, 17-40, 16-40, 15-40, 14-40, 13-40, 12-40, 11-40, 10-40, 9-40, 8-40, 7-40, 6-40, 5-40, 4-40, 3-40, 2-40, or 1-40 of SEQ ID NO: 3664, wherein the second spacer comprises at its 3′ end nucleotides 5-20 of SEQ ID NO:518, optionally wherein the second spacer comprises at its 3′ end SEQ ID NO:518.

Embodiment 58e. The prime editing composition of any one of Embodiments 50-58, wherein the second editing template comprises nucleotides 31-40, 30-40, 29-40, 28-40, 27-40, 26-40, 25-40, 24-40, 23-40, 22-40, 21-40, 20-40, 19-40, 18-40, 17-40, 16-40, 15-40, 14-40, 13-40, 12-40, 11-40, 10-40, 9-40, 8-40, 7-40, 6-40, 5-40, 4-40, 3-40, 2-40, or 1-40 of SEQ ID NO:3665, wherein the second spacer comprises at its 3′ end nucleotides 5-20 of SEQ ID NO:536, optionally wherein the second spacer comprises at its 3′ end SEQ ID NO:536.

Embodiment 58f: The prime editing composition of any one of Embodiments 50-58, wherein the second editing template comprises nucleotides 31-40, 30-40, 29-40, 28-40, 27-40, 26-40, 25-40, 24-40, 23-40, 22-40, 21-40, 20-40, 19-40, 18-40, 17-40, 16-40, 15-40, 14-40, 13-40, 12-40, 11-40, 10-40, 9-40, 8-40, 7-40, 6-40, 5-40, 4-40, 3-40, 2-40, or 1-40 of SEQ ID NO:3666, wherein the second spacer comprises at its 3′ end nucleotides 5-20 of SEQ ID NO:554, optionally wherein the second spacer comprises at its 3′ end SEQ ID NO:554.

Embodiment 58g: The prime editing composition of any one of Embodiments 50-58, wherein the second editing template comprises nucleotides 31-40, 30-40, 29-40, 28-40, 27-40, 26-40, 25-40, 24-40, 23-40, 22-40, 21-40, 20-40, 19-40, 18-40, 17-40, 16-40, 15-40, 14-40, 13-40, 12-40, 11-40, 10-40, 9-40, 8-40, 7-40, 6-40, 5-40, 4-40, 3-40, 2-40, or 1-40 of SEQ ID NO:3667, wherein the second spacer comprises at its 3′ end nucleotides 5-20 of SEQ ID NO:572, optionally wherein the second spacer comprises at its 3′ end SEQ ID NO:572.

Embodiment 58h: The prime editing composition of any one of Embodiments 50-58, wherein the second editing template comprises nucleotides 31-40, 30-40, 29-40, 28-40, 27-40, 26-40, 25-40, 24-40, 23-40, 22-40, 21-40, 20-40, 19-40, 18-40, 17-40, 16-40, 15-40, 14-40, 13-40, 12-40, 11-40, 10-40, 9-40, 8-40, 7-40, 6-40, 5-40, 4-40, 3-40, 2-40, or 1-40 of SEQ ID NO:3668, wherein the second spacer comprises at its 3′ end nucleotides 5-20 of SEQ ID NO:590, optionally wherein the second spacer comprises at its 3′ end SEQ ID NO:590.

Embodiment 58i: The prime editing composition of any one of Embodiments 50-58, wherein the second editing template comprises nucleotides 31-40, 30-40, 29-40, 28-40, 27-40, 26-40, 25-40, 24-40, 23-40, 22-40, 21-40, 20-40, 19-40, 18-40, 17-40, 16-40, 15-40, 14-40, 13-40, 12-40, 11-40, 10-40, 9-40, 8-40, 7-40, 6-40, 5-40, 4-40, 3-40, 2-40, or 1-40 of SEQ ID NO:3669, wherein the second spacer comprises at its 3′ end nucleotides 5-20 of SEQ ID NO:39, optionally wherein the second spacer comprises at its 3′ end SEQ ID NO:39.

Embodiment 58j: The prime editing composition of any one of Embodiments 50-58, wherein the second editing template comprises nucleotides 31-40, 30-40, 29-40, 28-40, 27-40, 26-40, 25-40, 24-40, 23-40, 22-40, 21-40, 20-40, 19-40, 18-40, 17-40, 16-40, 15-40, 14-40, 13-40, 12-40, 11-40, 10-40, 9-40, 8-40, 7-40, 6-40, 5-40, 4-40, 3-40, 2-40, or 1-40 of SEQ ID NO:3670, wherein the second spacer comprises at its 3′ end nucleotides 5-20 of SEQ ID NO:912, optionally wherein the second spacer comprises at its 3′ end SEQ ID NO:912.

Embodiment 58k: The prime editing composition of any one of Embodiments 50-58, wherein the second editing template comprises nucleotides 31-40, 30-40, 29-40, 28-40, 27-40, 26-40, 25-40, 24-40, 23-40, 22-40, 21-40, 20-40, 19-40, 18-40, 17-40, 16-40, 15-40, 14-40, 13-40, 12-40, 11-40, 10-40, 9-40, 8-40, 7-40, 6-40, 5-40, 4-40, 3-40, 2-40, or 1-40 of SEQ ID NO:3671, wherein the second spacer comprises at its 3′ end nucleotides 5-20 of SEQ ID NO:929, optionally wherein the second spacer comprises at its 3′ end SEQ ID NO:929.

Embodiment 58l: The prime editing composition of any one of Embodiments 50-58, wherein the second editing template comprises nucleotides 31-40, 30-40, 29-40, 28-40, 27-40, 26-40, 25-40, 24-40, 23-40, 22-40, 21-40, 20-40, 19-40, 18-40, 17-40, 16-40, 15-40, 14-40, 13-40, 12-40, 11-40, 10-40, 9-40, 8-40, 7-40, 6-40, 5-40, 4-40, 3-40, 2-40, or 1-40 of SEQ ID NO:3672, wherein the second spacer comprises at its 3′ end nucleotides 5-20 of SEQ ID NO:947, optionally wherein the second spacer comprises at its 3′ end SEQ ID NO:947.

Embodiment 58m: The prime editing composition of any one of Embodiments 50-58, wherein the second editing template comprises nucleotides 31-40, 30-40, 29-40, 28-40, 27-40, 26-40, 25-40, 24-40, 23-40, 22-40, 21-40, 20-40, 19-40, 18-40, 17-40, 16-40, 15-40, 14-40, 13-40, 12-40, 11-40, 10-40, 9-40, 8-40, 7-40, 6-40, 5-40, 4-40, 3-40, 2-40, or 1-40 of SEQ ID NO:3673, wherein the second spacer comprises at its 3′ end nucleotides 5-20 of SEQ ID NO:965, optionally wherein the second spacer comprises at its 3′ end SEQ ID NO:965.

Embodiment 58n: The prime editing composition of any one of Embodiments 50-58, wherein the second editing template comprises nucleotides 31-40, 30-40, 29-40, 28-40, 27-40, 26-40, 25-40, 24-40, 23-40, 22-40, 21-40, 20-40, 19-40, 18-40, 17-40, 16-40, 15-40, 14-40, 13-40, 12-40, 11-40, 10-40, 9-40, 8-40, 7-40, 6-40, 5-40, 4-40, 3-40, 2-40, or 1-40 of SEQ ID NO:3674, wherein the second spacer comprises at its 3′ end nucleotides 5-20 of SEQ ID NO:983, optionally wherein the second spacer comprises at its 3′ end SEQ ID NO:983.

Embodiment 58o: The prime editing composition of any one of Embodiments 50-58, wherein the second editing template comprises nucleotides 31-40, 30-40, 29-40, 28-40, 27-40, 26-40, 25-40, 24-40, 23-40, 22-40, 21-40, 20-40, 19-40, 18-40, 17-40, 16-40, 15-40, 14-40, 13-40, 12-40, 11-40, 10-40, 9-40, 8-40, 7-40, 6-40, 5-40, 4-40, 3-40, 2-40, or 1-40 of SEQ ID NO: 3675, wherein the second spacer comprises at its 3′ end nucleotides 5-20 of SEQ ID NO:1001, optionally wherein the second spacer comprises at its 3′ end SEQ ID NO:1001.

Embodiment 58p: The prime editing composition of any one of Embodiments 50-58, wherein the second editing template comprises nucleotides 31-40, 30-40, 29-40, 28-40, 27-40, 26-40, 25-40, 24-40, 23-40, 22-40, 21-40, 20-40, 19-40, 18-40, 17-40, 16-40, 15-40, 14-40, 13-40, 12-40, 11-40, 10-40, 9-40, 8-40, 7-40, 6-40, 5-40, 4-40, 3-40, 2-40, or 1-40 of SEQ ID NO:3676, wherein the second spacer comprises at its 3′ end nucleotides 5-20 of SEQ ID NO:1019, optionally wherein the second spacer comprises at its 3′ end SEQ ID NO:1019.

Embodiment 58a: The prime editing composition of any one of Embodiments 50-58, wherein the second editing template comprises nucleotides 31-40, 30-40, 29-40, 28-40, 27-40, 26-40, 25-40, 24-40, 23-40, 22-40, 21-40, 20-40, 19-40, 18-40, 17-40, 16-40, 15-40, 14-40, 13-40, 12-40, 11-40, 10-40, 9-40, 8-40, 7-40, 6-40, 5-40, 4-40, 3-40, 2-40, or 1-40 of SEQ ID NO:3677, wherein the second spacer comprises at its 3′ end nucleotides 5-20 of SEQ ID NO:1341, optionally wherein the second spacer comprises at its 3′ end SEQ ID NO:1341.

Embodiment 58r: The prime editing composition of any one of Embodiments 50-58, wherein the second editing template comprises nucleotides 31-40, 30-40, 29-40, 28-40, 27-40, 26-40, 25-40, 24-40, 23-40, 22-40, 21-40, 20-40, 19-40, 18-40, 17-40, 16-40, 15-40, 14-40, 13-40, 12-40, 11-40, 10-40, 9-40, 8-40, 7-40, 6-40, 5-40, 4-40, 3-40, 2-40, or 1-40 of SEQ ID NO:3678, wherein the second spacer comprises at its 3′ end nucleotides 5-20 of SEQ ID NO:20, optionally wherein the second spacer comprises at its 3′ end SEQ ID NO:20.

Embodiment 58s: The prime editing composition of any one of Embodiments 50-58, wherein the second editing template comprises nucleotides 31-40, 30-40, 29-40, 28-40, 27-40, 26-40, 25-40, 24-40, 23-40, 22-40, 21-40, 20-40, 19-40, 18-40, 17-40, 16-40, 15-40, 14-40, 13-40, 12-40, 11-40, 10-40, 9-40, 8-40, 7-40, 6-40, 5-40, 4-40, 3-40, 2-40, or 1-40 of SEQ ID NO:3679, wherein the second spacer comprises at its 3′ end nucleotides 5-20 of SEQ ID NO:77, optionally wherein the second spacer comprises at its 3′ end SEQ ID NO:77.

Embodiment 58t: The prime editing composition of any one of Embodiments 50-58, wherein the second editing template comprises nucleotides 31-40, 30-40, 29-40, 28-40, 27-40, 26-40, 25-40, 24-40, 23-40, 22-40, 21-40, 20-40, 19-40, 18-40, 17-40, 16-40, 15-40, 14-40, 13-40, 12-40, 11-40, 10-40, 9-40, 8-40, 7-40, 6-40, 5-40, 4-40, 3-40, 2-40, or 1-40 of SEQ ID NO:3680, wherein the second spacer comprises at its 3′ end nucleotides 5-20 of SEQ ID NO:484, optionally wherein the second spacer comprises at its 3′ end SEQ ID NO:484.

Embodiment 58u: The prime editing composition of any one of Embodiments 50-58, wherein the second editing template comprises nucleotides 31-40, 30-40, 29-40, 28-40, 27-40, 26-40, 25-40, 24-40, 23-40, 22-40, 21-40, 20-40, 19-40, 18-40, 17-40, 16-40, 15-40, 14-40, 13-40, 12-40, 11-40, 10-40, 9-40, 8-40, 7-40, 6-40, 5-40, 4-40, 3-40, 2-40, or 1-40 of SEQ ID NO:3681, wherein the second spacer comprises at its 3′ end nucleotides 5-20 of SEQ ID NO:58, optionally wherein the second spacer comprises at its 3′ end SEQ ID NO:58.

Embodiment 58v: The prime editing composition of any one of Embodiments 50-58, wherein the second editing template comprises nucleotides 31-40, 30-40, 29-40, 28-40, 27-40, 26-40, 25-40, 24-40, 23-40, 22-40, 21-40, 20-40, 19-40, 18-40, 17-40, 16-40, 15-40, 14-40, 13-40, 12-40, 11-40, 10-40, 9-40, 8-40, 7-40, 6-40, 5-40, 4-40, 3-40, 2-40, or 1-40 of SEQ ID NO:3682, wherein the second spacer comprises at its 3′ end nucleotides 5-20 of SEQ ID NO:1, optionally wherein the second spacer comprises at its 3′ end SEQ ID NO:1.

Embodiment 59: The prime editing composition of Embodiment 57 or 58, wherein the first PEgRNA comprises a sequence selected from the group consisting of SEQ ID NOs: 503-517, and wherein the second PEgRNA comprises a sequence selected from the group consisting of SEQ ID NOs: 1548-1562.

Embodiment 60: The prime editing composition of any one of Embodiments 1-28, wherein the first editing template comprises from 5′ to 3′ (i) a region of complementarity to the second editing template and (ii) nucleotides 8-17 of the selected sequence for the second spacer; and wherein the second editing template comprises from 5′ to 3′ (i) a region of complementarity to the first editing template and (ii) nucleotides 8-17 of the selected sequence for the first spacer.

Embodiment 61: The prime editing composition of Embodiment 60, wherein the first editing template comprises nucleotides 1-17, 2-17, 3-17, 4-17, 5-17, 6-17, 7-17, or 8-17 of the selected sequence for the second spacer, and/or wherein the second editing template comprises 1-17, 2-17, 3-17, 4-17, 5-17, 6-17, 7-17, or 8-17 of the selected sequence for the first spacer.

Embodiment 62: The prime editing composition of Embodiment 60, wherein the first editing template comprises nucleotides 3-17 of the selected sequence for the second spacer, and wherein the second editing template comprises nucleotides 3-17 of the selected sequence of the first spacer.

Embodiment 63: The prime editing composition of Embodiment 60, wherein the first editing template comprises a region of complementarity to a sequence on the second strand of the DMPK gene that is directly 3′ to nucleotide 3 of the second search target sequence, wherein the region of complementarity is about 20, about 25, about 30, about 35, about 40, about 45, or about 50 nucleotides in length.

Embodiment 64: The prime editing composition of Embodiment 63, wherein the region of complementarity between the first editing template and the sequence on the second strand of the DMPK gene is about 20-30 nucleotides in length.

Embodiment 65: The prime editing composition of Embodiment 60, wherein the second editing template comprises a region of complementarity to a sequence on the first strand of the DMPK gene that is directly 3′ to nucleotide 3 of the first search target sequence, wherein the region of complementarity is about 20, about 25, about 30, about 35, about 40, about 45, or about 50 nucleotides in length.

Embodiment 66: The prime editing composition of Embodiment 65, wherein the region of complementarity between the second editing template and the sequence on the first strand of the DMPK gene is about 20-30 nucleotides in length.

Embodiment 67: The prime editing composition of any one of Embodiments 60-66, wherein the region of complementarity between the first editing template and the second editing template is about 20 to about 80 nucleotides in length.

In some embodiments, a prime editing composition of any one of Embodiments 60-66 comprises a region of complementarity between the first editing template and the second editing template that is about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, or about 80 nucleotides in length.

In some embodiments, a prime editing composition of any one of Embodiments 60-66 comprises a region of complementarity between the first editing template and the second editing template that is between 20 and 80 nucleotides; between 21 and 80 nucleotides; between 22 and 80 nucleotides; between 23 and 80 nucleotides; between 24 and 80 nucleotides; between 25 and 80 nucleotides; between 26 and 80 nucleotides; between 27 and 80 nucleotides; between 28 and 80 nucleotides; between 29 and 80 nucleotides; between 30 and 80 nucleotides; between 31 and 80 nucleotides; between 32 and 80 nucleotides; between 33 and 80 nucleotides; between 34 and 80 nucleotides; between 35 and 80 nucleotides; between 36 and 80 nucleotides; between 37 and 80 nucleotides; between 38 and 80 nucleotides; between 39 and 80 nucleotides; between 40 and 80 nucleotides; between 41 and 80 nucleotides; between 42 and 80 nucleotides; between 43 and 80 nucleotides; between 44 and 80 nucleotides; between 45 and 80 nucleotides; between 46 and 80 nucleotides; between 47 and 80 nucleotides; between 48 and 80 nucleotides; between 49 and 80 nucleotides; between 50 and 80 nucleotides; between 51 and 80 nucleotides; between 52 and 80 nucleotides; between 53 and 80 nucleotides; between 54 and 80 nucleotides; between 55 and 80 nucleotides; between 56 and 80 nucleotides; between 57 and 80 nucleotides; between 58 and 80 nucleotides; between 59 and 80 nucleotides; between 60 and 80 nucleotides; between 61 and 80 nucleotides; between 62 and 80 nucleotides; between 63 and 80 nucleotides; between 64 and 80 nucleotides; between 65 and 80 nucleotides; between 66 and 80 nucleotides; between 67 and 80 nucleotides; between 68 and 80 nucleotides; between 69 and 80 nucleotides; between 70 and 80 nucleotides; between 71 and 80 nucleotides; between 72 and 80 nucleotides; between 73 and 80 nucleotides; between 74 and 80 nucleotides; between 75 and 80 nucleotides; between 76 and 80 nucleotides; between 77 and 80 nucleotides; between 78 and 80 nucleotides; between 20 and 80 nucleotides; between 20 and 79 nucleotides; between 20 and 78 nucleotides; between 20 and 77 nucleotides; between 20 and 76 nucleotides; between 20 and 75 nucleotides; between 20 and 74 nucleotides; between 20 and 73 nucleotides; between 20 and 72 nucleotides; between 20 and 71 nucleotides; between 20 and 70 nucleotides; between 20 and 69 nucleotides; between 20 and 68 nucleotides; between 20 and 67 nucleotides; between 20 and 66 nucleotides; between 20 and 65 nucleotides; between 20 and 64 nucleotides; between 20 and 63 nucleotides; between 20 and 62 nucleotides; between 20 and 61 nucleotides; between 20 and 60 nucleotides; between 20 and 59 nucleotides; between 20 and 58 nucleotides; between 20 and 57 nucleotides; between 20 and 56 nucleotides; between 20 and 55 nucleotides; between 20 and 54 nucleotides; between 20 and 53 nucleotides; between 20 and 52 nucleotides; between 20 and 51 nucleotides; between 20 and 50 nucleotides; between 20 and 49 nucleotides; between 20 and 48 nucleotides; between 20 and 47 nucleotides; between 20 and 46 nucleotides; between 20 and 45 nucleotides; between 20 and 44 nucleotides; between 20 and 43 nucleotides; between 20 and 42 nucleotides; between 20 and 41 nucleotides; between 20 and 40 nucleotides; between 20 and 39 nucleotides; between 20 and 38 nucleotides; between 20 and 37 nucleotides; between 20 and 36 nucleotides; between 20 and 35 nucleotides; between 20 and 34 nucleotides; between 20 and 33 nucleotides; between 20 and 32 nucleotides; between 20 and 31 nucleotides; between 20 and 30 nucleotides; between 20 and 29 nucleotides; between 20 and 28 nucleotides; between 20 and 27 nucleotides; between 20 and 26 nucleotides; between 20 and 25 nucleotides; between 20 and 24 nucleotides; between 20 and 23 nucleotides; or between 20 and 22 nucleotides in length.

In some embodiments, a prime editing composition of any one of Embodiments 60-66 comprises a region of complementarity between the first editing template and the second editing template that is at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, or at least 80 nucleotides in length.

In some embodiments, a prime editing composition of any one of Embodiments 60-66 comprises a region of complementarity between the first editing template and the second editing template that is no more than 20, no more than 21, no more than 22, no more than 23, no more than 24, no more than 25, no more than 26, no more than 27, no more than 28, no more than 29, no more than 30, no more than 31, no more than 32, no more than 33, no more than 34, no more than 35, no more than 36, no more than 37, no more than 38, no more than 39, no more than 40, no more than 41, no more than 42, no more than 43, no more than 44, no more than 45, no more than 46, no more than 47, no more than 48, no more than 49, no more than 50, no more than 51, no more than 52, no more than 53, no more than 54, no more than 55, no more than 56, no more than 57, no more than 58, no more than 59, no more than 60, no more than 61, no more than 62, no more than 63, no more than 64, no more than 65, no more than 66, no more than 67, no more than 68, no more than 69, no more than 70, no more than 71, no more than 72, no more than 73, no more than 74, no more than 75, no more than 76, no more than 77, no more than 78, no more than 79, or no more than 80 nucleotides in length.

Embodiment 68: The prime editing composition of Embodiment 67, wherein the region of complementarity between the first editing template and the second editing template is about 20 to about 40 nucleotides in length.

Embodiment 69: The prime editing composition of Embodiment 67, wherein the region of complementarity between the first editing template and the second editing template is 23-38 nucleotides in length.

Embodiment 70: A prime editing composition comprising: a first prime editing guide RNA (PEgRNA) or one or more polynucleotides encoding the first PEgRNA and a second PEgRNA or one or more polynucleotides encoding the second PEgRNA, wherein (a) the first PEgRNA comprises: (i) a first spacer comprising at its 3′ end nucleotides 5-20 of a Spacer sequence selected from any one of Tables 1A, 2A, 3A, 4A, 5A, 6A, 7A, 8A, 9A, 10A, 11A, 12A, 13A, 14A, 15A, 16A, 17A, 18A, and 19A; (ii) a first gRNA core capable of binding to a Cas9 protein; (iii) a first extension arm comprising: a first primer binding site (PBS) comprising a PBS sequence selected from the same Table as the first spacer, and a first editing template comprising an RTT sequence selected from Table 37 and has an RTT Paring NO: x in Table 37; and (b) the second PEgRNA comprises: (i) a second spacer comprising at its 3′ end nucleotides 5-20 of a Spacer sequence selected from any one of Tables 20A, 21A, 22A, 23A, 24A, 25A, 26A, 27A, 28A, 29A, 30A, 31A, 32A, 33A, 34A, 35A, and 36A; (ii) a second gRNA core capable of binding to a Cas9 protein; (iii) a second extension arm comprising: a second primer binding site (PBS) comprising a PBS sequence selected from the same Table as the second spacer, and a second editing template comprising an RTT sequence selected from Table 38 and has an RTT paring NO: x in Table 38; wherein the RTT paring number x in (a) and (b) are the same integer.

Embodiment 71: The prime editing composition of Embodiment 70, wherein the first editing template and the second editing template have the same length and are perfectly complementary to each other.

Embodiment 72: A prime editing composition comprising a first prime editing guide RNA (PEgRNA) or a one or more polynucleotides encoding the first PEgRNA and a second PEgRNA or one or more polynucleotides encoding the second PEgRNA, wherein (a) the first PEgRNA comprises: (i) a first spacer comprising at its 3′ end nucleotides 5-20 of a Spacer sequence selected from Table xA, wherein x is an integer from 1 to 19; (ii) a first gRNA core capable of binding to a Cas9 protein; (iii) a first extension arm comprising: a first primer binding site (PBS) comprising a PBS sequence selected from the same Table as the first spacer, and a first editing template comprising an RTT sequence selected from Table 39, wherein the Paring Spacer number of the RTT sequence in Table 39 is y; and (b) the second PEgRNA comprises: (iv) a second spacer comprising at its 3′ end nucleotides 5-20 of a Spacer sequence selected from Table yA, wherein y is an integer from 20 to 36; (v) a second gRNA core capable of binding to a Cas9 protein; (vi) a second extension arm comprising: a second editing template comprising an RTT sequence selected from Table 40, wherein the Paring Spacer number of the RTT sequence in Table 40 is x, and a second primer binding site (PBS) comprising a PBS sequence selected from the same Table as the second spacer, wherein x in (a) and (b) are the same integer, and wherein y in (a) and (b) are the same integer.

Embodiment 73: The prime editing composition of any one of Embodiments 70-72, wherein the first spacer and/or the second spacer is from 16 to 22 nucleotides in length.

Embodiment 74: The prime editing composition of any one of Embodiments 70-73, wherein the first spacer and/or the second spacer comprises at its 3′ the selected sequence.

Embodiment 75: The prime editing composition of Embodiment 74, wherein the first spacer and/or the second spacer is 20 nucleotides in length.

Embodiment 76: The prime editing composition of any one of Embodiments 70-75, wherein the first gRNA core and the second gRNA core comprise the same sequence.

Embodiment 77: The prime editing composition of Embodiment 76, wherein the first gRNA core and the second gRNA core each comprises SEQ ID NO: 3641.

Embodiment 78: The prime editing composition of any one of Embodiments 70-77, wherein the first spacer, the first gRNA core, the first editing template, and the first PBS form a contiguous sequence in a single molecule.

Embodiment 79: The prime editing composition of Embodiment 78, wherein the first pegRNA comprises from 5′ to 3′ the first spacer, the first gRNA core, the first editing template, and the first PBS.

Embodiment 80: The prime editing composition of any one of Embodiments 70-79, wherein the second spacer, the second gRNA core, the second editing template, and the second PBS form a contiguous sequence in a single molecule.

Embodiment 81: The prime editing composition of Embodiment 80, wherein the second pegRNA comprises from 5′ to 3′ the second spacer, the second gRNA core, the second editing template, and the second PBS.

Embodiment 82: The prime editing composition of any one of Embodiments 1-81, wherein the first PEgRNA and/or the second PEgRNA further comprises 3′ mN*mN*mN*N and 5′mN*mN*mN*modifications, where m indicates that the nucleotide contains a 2′-O-Me modification and a * indicates the presence of a phosphorothioate bond.

Embodiment 83: The prime editing composition of any one of Embodiments 1-82, further comprising a prime editor or one or more polynucleotides encoding the prime editor, wherein the prime editor comprises a Cas9 nickase having a nuclease inactivating mutation in the HNH domain and a reverse transcriptase.

Embodiment 84: The prime editing composition of Embodiment 83, wherein the Cas9 nickase comprises an amino acid sequence having at least 80%, 85%, 90%, 95%, 96%, 97%, 98%, 99%, or 100% identity to SEQ ID NO: 3582.

Embodiment 85: The prime editing composition of Embodiment 83 or 84, wherein the reverse transcriptase comprises an amino acid sequence having at least 80%, 85%, 90%, 95%, 96%, 97%, 98%, 99%, or 100% identity to SEQ ID NO: 3579.

Embodiment 86: The prime editing system of Embodiment 84 or 85, wherein the sequence identities are determined by Needleman-Wunsch alignment of two protein sequences with Gap Costs set to Existence: 11 Extension: 1 where percent identity is calculated by dividing the number of identities by the length of the alignment.

Embodiment 87: The prime editing composition of any one of Embodiments 83-86, wherein the prime editor is a fusion protein.

Embodiment 88: The prime editing composition of any one of Embodiments 83-86, wherein the one or more polynucleotides comprise (a) a first sequence encoding an N-terminal portion of the Cas9 nickase and an intein-N and (b) a second sequence encoding a intein-C, a C-terminal portion of the Cas9 nickase, and the reverse transcriptase.

Embodiment 89: The prime editing composition of any one of Embodiments 83-88, comprising one or more vectors comprising the one or more polynucleotides encoding the first PEgRNA, the one or more polynucleotides encoding the second PEgRNA, and the one or more polynucleotides encoding the prime editor.

Embodiment 90: The prime editing composition of Embodiment 89, wherein the one or more vectors are AAV vectors.

Embodiment 91: An LNP comprising the prime editing composition of any one of Embodiments 1-90.

Embodiment 92: A method of editing a DMPK gene, the method comprising contacting the DMPK gene with (a) the prime editing composition of any one of Embodiments 1-82 and a prime editor comprising a Cas9 nickase having a nuclease inactivation mutation in the HNH domain and a reverse transcriptase, (b) the prime editing composition of any one of Embodiments 83-90, or (c) the LNP of Embodiment 91.

Embodiment 93: The method of Embodiment 92, wherein the DMPK gene is in a cell.

Embodiment 94: The method of Embodiment 93, wherein the cell is a mammalian cell.

Embodiment 95: The method of Embodiment 94, wherein the cell is a human cell.

Embodiment 96: The method of any one of Embodiments 93-95, wherein the cell is a fibroblast, a myoblast, a myosatellite, a muscle progenitor cell, a cardiomyocyte, a differentiated muscle cell, a skeletal muscle cell, or a smooth muscle cell.

Embodiment 97: The method of any one of Embodiments 93-96, wherein the cell is in a subject.

Embodiment 98: The method of Embodiment 87, wherein the subject is a human.

Embodiment 99: The method of any one of Embodiments 93-98, wherein the cell is from a subject having myotonic dystrophy type 1.

Embodiment 100: A cell generated by the method of any one of Embodiments 92-99.

Embodiment 101: A population of cells generated by the method of any one of Embodiments 92-99.

Embodiment 102: A method for treating myotonic dystrophy type 1 in a subject in need thereof, the method comprising administering to the subject (a) the prime editing composition of any one of Embodiments 1-82 and a prime editor comprising a Cas9 nickase having a nuclease inactivation mutation in the HNH domain and a reverse transcriptase, (b) the prime editing composition of any one of Embodiments 83-90, (c) the LNP of Embodiment 91, (d) the cell of Embodiment 00, or (e) the population of cells of Embodiment 101.

Embodiment 103: A composition comprising a first prime editing guide RNA (PEgRNA) and a second PEgRNA, wherein: the first PEgRNA comprises a first spacer that is complementary to a first search target sequence on a first strand of a double-stranded DMPK gene, a first gRNA core that associates with a first prime editor comprising a DNA binding domain and DNA polymerase domain, and a first editing template; and the second PEgRNA comprises a second spacer that is complementary to a second search target sequence on a second strand of the double-stranded DMPK gene, a second gRNA core that associates with a second prime editor comprising a DNA binding domain and a DNA polymerase domain, and a second editing template; wherein the first strand and the second strand of the double-stranded DMPK gene are complementary to each other, and wherein the first editing template and the second editing template each comprises a region of complementarity to each other.

Embodiment 104: A composition comprising a first prime editing guide RNA (PEgRNA) and a second PEgRNA, wherein: the first PEgRNA comprises a first spacer that is complementary to a first search target sequence on a first strand of a double-stranded DMPK gene, a first gRNA core that associates with a first prime editor comprising a DNA binding domain and DNA polymerase domain, and a first editing template; and the second PEgRNA comprises a second spacer that is complementary to a second search target sequence on a second strand of the double-stranded DMPK gene, a second gRNA core that associates with a second prime editor comprising a DNA binding domain and a DNA polymerase domain, and a second editing template; wherein the first strand and the second strand of the double-stranded DMPK gene are complementary to each other, wherein the first editing template comprises a region of identity to a sequence on the first strand of the DMPK gene, and wherein the second editing template comprises a region of identity to a sequence on the second strand of the double-stranded DMPK gene.

Embodiment 105: The composition of Embodiment 103 or 104, wherein the first PEgRNA directs the first prime editor to generate a first nick on the second strand of the DMPK gene, wherein the second PEgRNA directs the second prime editor to generate a second nick on the first strand of the DMPK gene, and wherein the DMPK gene comprises an inter-nick duplex (IND) between the position of the first nick and the position of the second nick.

Embodiment 106: The composition of Embodiment 105, wherein the IND comprises an array of tri-nucleotide repeats.

Embodiment 107: The composition of any one of the Embodiments 103-106, wherein the double-stranded DMPK gene comprises a mutation associated with myotonic dystrophy.

Embodiment 108: The composition of Embodiment 107, wherein the IND comprises the mutation associated with myotonic dystrophy.

Embodiment 109: The composition of Embodiment 108, wherein the mutation is an increased number of tri-nucleotide repeats in the array of tri-nucleotide repeats compared to a wild type DMPK gene.

Embodiment 110: The composition of any one of Embodiments 106-109, wherein the array of tri-nucleotide repeats comprises the sequence (CTG)n or a complementary sequence thereof, wherein n is any integer greater than 34.

Embodiment 111: The composition of Embodiment 110, wherein n is an integer greater than 50.

Embodiment 112: The composition of Embodiment 111, wherein n is an integer greater than 100.

Embodiment 113: The composition of any one of Embodiments 103 or 105-112, wherein the first editing template comprises an exogenous sequence compared to the DMPK gene.

Embodiment 114: The composition of any one of Embodiments 103 or 105-112, wherein the second editing template comprises an exogenous sequence compared to the DMPK gene.

Embodiment 115: The composition of Embodiment 113 or 114, wherein the region of complementarity between the first editing template and the second editing template comprises an exogenous sequence compared to the DMPK gene.

Embodiment 116: The composition of any one of Embodiments 113-115, wherein the exogenous sequence comprises a marker, an expression tag, a barcode, or a regulatory sequence.

Embodiment 117: The composition of any one of Embodiments 103 or 105-112, wherein the first editing template comprises a region of complementarity to the IND on the second strand of the DMPK gene.

Embodiment 118: The composition of any one of Embodiments 103 or 105-112, wherein the second editing template comprises a region of complementarity to the IND on the first strand of the DMPK gene.

Embodiment 119: The composition of Embodiment 117 or 118, wherein the sequence of the region of complementarity between the first editing template and the second editing template is at least partially identical to a sequence in the IND.

Embodiment 120: The composition of any one of Embodiments 117-119, wherein the first editing template comprises the sequence (CAG)n, wherein n is any integer between 0 and 33.

Embodiment 121: The composition of Embodiment 120, wherein n is any integer between 5 and 30.

Embodiment 122: The composition of Embodiment 120, wherein n is any integer between 10 and 25.

Embodiment 123: The composition of any one of Embodiments 117-119, wherein the second editing template comprises the sequence (CUG)m, wherein m is any integer between 0 and 33.

Embodiment 124: The composition of Embodiment 123, wherein m is any integer between 5 and 30.

Embodiment 125: The composition of Embodiment 123, wherein m is any integer between 10 and 25.

Embodiment 126: The composition of Embodiment 119, wherein the region of complementarity between the first editing template and the second editing template comprises the sequence (CAG)w, wherein w is any integer between 0 and 33.

Embodiment 127: The composition of Embodiment 126, wherein w is any integer between 5 and 30.

Embodiment 128: The composition of Embodiment 126, wherein w is any integer between 10 and 25.

Embodiment 129: The composition of any one of Embodiments 120-128, wherein (n+m−w) is an integer no greater than 33.

Embodiment 130: The composition of any one of the preceding Embodiments 103-129, wherein the IND further comprises a sequence upstream of the array of tri-nucleotide repeats, wherein upstream is determined by referring to the 5′ to 3′ orientation of the coding strand of the DMPK gene.

Embodiment 131: The composition of Embodiment 130, wherein the sequence upstream of the tri-nucleotide repeat sequence is at least 10 base pairs in length.

Embodiment 132: The composition of Embodiment 131, wherein the sequence upstream of the tri-nucleotide repeat sequence is 5 to 25 base pairs in length.

Embodiment 133: The composition of Embodiment 131, wherein the sequence upstream of the tri-nucleotide repeat sequence is 20 to 50 base pairs in length.

Embodiment 134: The composition of Embodiment 131, wherein the sequence upstream of the tri-nucleotide repeat sequence is 50 to 100 base pairs in length.

Embodiment 135: The composition of Embodiment 131, wherein the sequence upstream of the tri-nucleotide repeat sequence is 100, 200, 300, 400, or 500 base pairs in length.

Embodiment 136: The composition of any one of Embodiments 130-135, wherein the IND further comprises a sequence downstream of the array of tri-nucleotide repeats, wherein downstream is determined by referring to the 5′ to 3′ orientation of the coding strand of the DMPK gene.

Embodiment 137: The composition of Embodiment 136, wherein the sequence downstream of the tri-nucleotide repeat sequence is at least 10 base pairs in length.

Embodiment 138: The composition of Embodiment 136, wherein the sequence downstream of the tri-nucleotide repeat sequence is 5 to 25 base pairs in length.

Embodiment 139: The composition of Embodiment 136, wherein the sequence downstream of the tri-nucleotide repeat sequence is 20 to 50 base pairs in length.

Embodiment 140: The composition of Embodiment 136, wherein the sequence downstream of the tri-nucleotide repeat sequence is 50 to 100 base pairs in length.

Embodiment 141: The composition of Embodiment 136, wherein the sequence downstream of the tri-nucleotide repeat sequence is 100, 200, 300, 400, or 500 base pairs in length.

Embodiment 142: The composition of any one of Embodiments 117-141, wherein the first editing template further comprises a region of complementarity to a sequence on the second strand of the DMPK gene that is upstream of the array of tri-nucleotide repeats and/or a region of complementarity to a sequence on the second strand of the DMPK gene that is downstream of the array of tri-nucleotide repeats.

Embodiment 143: The composition of Embodiment 142, wherein the region of complementarity of the first editing template to the sequence upstream of the array of trinucleotide repeats or the region of complementarity of the first editing template to the sequence downstream of the array of tri-nucleotide repeats is 5 to 25 nucleotides in length.

Embodiment 144: The composition of Embodiment 142, wherein the region of complementarity of the first editing template to the sequence upstream of the array of trinucleotide repeats or the region of complementarity of the first editing template to the sequence downstream of the array of tri-nucleotide repeats is 10 to 15 nucleotides in length.

Embodiment 145: The composition of Embodiment 142, wherein the region of complementarity of the first editing template to the sequence upstream of the array of trinucleotide repeats or the region of complementarity of the first editing template to the sequence downstream of the array of tri-nucleotide repeats is 20 to 50 nucleotides in length.

Embodiment 146: The composition of Embodiment 142, wherein the region of complementarity of the first editing template to the sequence upstream of the array of trinucleotide repeats or the region of complementarity of the first editing template to the sequence downstream of the array of tri-nucleotide repeats is 50 to 100 nucleotides in length.

Embodiment 147: The composition of any one of Embodiments 142-146, wherein the first editing template comprises a sequence selected from the group consisting of SEQ ID NO.s 3557-3576.

Embodiment 148: The composition of any one of Embodiments 142-147, wherein the first editing template comprises a sequence selected from the group consisting of SEQ ID NO.s 3537-3556-.

Embodiment 149: The composition of any one of Embodiments 117-148, wherein the second editing template further comprises a region of complementarity to a sequence on the first strand of the DMPK gene that is upstream of the array of tri-nucleotide repeats and/or a region of complementarity to a sequence on the first strand of the DMPK gene that is downstream of the array of tri-nucleotide repeats.

Embodiment 150: The composition of Embodiment 149, wherein the region of complementarity of the second editing template to the sequence upstream of the array of trinucleotide repeats or the region of complementarity of the second editing template to the sequence downstream of the array of tri-nucleotide repeats is 5 to 25 nucleotides in length.

Embodiment 151: The composition of Embodiment 149, wherein the region of complementarity of the second editing template to the sequence upstream of the array of trinucleotide repeats or the region of complementarity of the second editing template to the sequence downstream of the array of tri-nucleotide repeats is 10 to 15 nucleotides in length.

Embodiment 152: The composition of Embodiment 149, wherein the region of complementarity of the second editing template to the sequence upstream of the array of trinucleotide repeats or the region of complementarity of the second editing template to the sequence downstream of the array of tri-nucleotide repeats is 20 to 50 nucleotides in length.

Embodiment 153: The composition of Embodiment 149, wherein the region of complementarity of the second editing template to the sequence upstream of the array of trinucleotide repeats or the region of complementarity of the second editing template to the sequence downstream of the array of tri-nucleotide repeats is 50 to 100 nucleotides in length.

Embodiment 154: The composition of any one of Embodiments 149-153, wherein the second editing template comprises a sequence selected from the group consisting of SEQ ID Nos 3517-3526 and 3527-3536.

Embodiment 155: The composition of any one of Embodiments 149-154, wherein the second editing template comprises a sequence selected from the group consisting of SEQ ID Nos 3497-3516.

Embodiment 156: The composition of Embodiment 104-112, wherein the first editing template and the second editing template are not complementary to each other.

Embodiment 157: The composition of Embodiment 104-112 or 156, wherein the first editing template and the second editing template comprise a region of complementarity to each other.

Embodiment 158: The composition of Embodiment 157, wherein the region of complementarity between the first editing template and the second editing template comprises an exogenous sequence compared to the double-stranded DMPK gene.

Embodiment 159: The composition of Embodiment 158, wherein the exogenous sequence comprises a marker, an expression tag, a barcode, or a regulatory sequence.

Embodiment 160: The composition of any one of Embodiments 156-159, wherein the first editing template comprises a region of identity or substantial identity to a sequence on the first strand of the double-stranded DMPK gene immediately adjacent to and outside the IND.

Embodiment 161: The composition of Embodiment 158, wherein the region of identity or substantial identity of the first editing template to the sequence on the first strand of the double-stranded DMPK gene immediately adjacent to and outside the IND is at least 10 nucleotides in length.

Embodiment 162: The composition of Embodiment 160, wherein the region of identity or substantial identity of the first editing template to the sequence on the first strand of the double-stranded DMPK gene immediately adjacent to and outside the IND is 15 to 100 nucleotides in length.

Embodiment 163: The composition of any one of Embodiments 160-162, wherein the second editing template comprises a region of identity or substantial identity to a sequence on the second strand of the DMPK gene immediately adjacent to and outside the IND.

Embodiment 164: The composition of Embodiment 163, wherein the region of identity or substantial identity of the second editing template to the sequence on the second strand of the double-stranded DMPK gene immediately adjacent to and outside the IND is at least 10 nucleotides in length.

Embodiment 165: The composition of Embodiment 163, wherein the region of identity or substantial identity of the second editing template to the sequence on the second strand of the double-stranded DMPK gene immediately adjacent to and outside the IND is 15 to 100 nucleotides in length.

Embodiment 166: The composition of any one of the preceding Embodiments, wherein the first PEgRNA comprises a first primer binding site (PBS) sequence that comprises a region of complementarity to the second strand of the double-stranded DMPK gene.

Embodiment 167: The composition of Embodiment 166, wherein the second PEgRNA comprises a second PBS sequence that comprises a region of complementarity to the first strand of the double-stranded DMPK gene.

Embodiment 168: The composition of Embodiment 166 or 167, wherein the first PEgRNA comprises a structure: 5′-[first spacer]-[first gRNA core]-[first editing template]-[first primer binding site sequence]-3′.

Embodiment 169: The composition of Embodiment 166 or 167, wherein the first PEgRNA comprises a structure: 5′-[first editing template]-[first primer binding site sequence]-[first spacer]-[first gRNA core]-3′.

Embodiment 170: The composition of any one of Embodiments 166-169, wherein the second PEgRNA comprises a structure: 5′-[second spacer sequence]-[second gRNA core]-[second editing template]-[second primer binding site]-3′.

Embodiment 171: The composition of any one of Embodiments 166-169, wherein the second PEgRNA comprises a structure: 5′-[second editing template]-[second primer binding site sequence]-[second spacer]-[second gRNA core]-3′.

Embodiment 172: The composition of any one of Embodiments 167-171, wherein the first PBS is at least partially complementary to the first spacer sequence.

Embodiment 173: The composition of any one of Embodiments 167-172, wherein the second PBS is at least partially complementary to the second spacer sequence.

Embodiment 174: The composition of any one of the preceding Embodiments, wherein the 5′ end of the first search target sequence is about 10, 50, 100, 200, 300, 400, 500, 1000, 1500, 2000, 2500, or 3000 nucleotides downstream of 3′ end of the CAG repeats.

Embodiment 175: The composition of Embodiment 174, wherein the 5′ end of the first search target sequence is at least 100 nucleotides downstream of 3′ end of the CAG repeats.

Embodiment 176: The composition of Embodiment 174, wherein the 5′ end of the first search target sequence is at least 50 nucleotides downstream of 3′ end of the CAG repeats.

Embodiment 177: The composition of any one of the preceding Embodiments 103-176, wherein the 5′ end of the second search target sequence is about 10, 50, 100, 200, 300, 400, 500, 1000, 1500, 2000, 2500, or 3000 nucleotides downstream of 3′ end of the CTG repeats.

Embodiment 178: The composition of Embodiment 177, wherein the 5′ end of the second search target sequence is at least 100 nucleotides downstream of 3′ end of the CTG repeats.

Embodiment 179: The composition of Embodiment 177, wherein the 5′ end of the second search target sequence is at least 50 nucleotides downstream of 3′ end of the CTG repeats.

Embodiment 180: The composition of any one of Embodiments 166-179, wherein the first PBS is about 2 to 20 nucleotides in length.

Embodiment 181: The composition of any one of Embodiments 166-180, wherein the second PBS is about 2 to 20 nucleotides in length.

Embodiment 182: The composition of Embodiment 181, wherein the first PBS is about 8 to 16 nucleotides in length.

Embodiment 183: The composition of Embodiment 181, wherein the second PBS is about 8 to 16 nucleotides in length.

Embodiment 184: The composition of Embodiment 181, wherein the first editing template is about 15-150 nucleotides in length.

Embodiment 185: The composition of Embodiment 181, wherein the first editing template is about 15-100 nucleotides in length.

Embodiment 186: The composition of Embodiment 181, wherein the first editing template is about 30-100 nucleotides in length.

Embodiment 187: The composition of Embodiment 181, wherein the first editing template is about 50-100 nucleotides in length.

Embodiment 188: The composition of Embodiment 181, wherein the first editing template is about 15-50 nucleotides in length.

Embodiment 189: The composition of Embodiment 181, wherein the second editing template is about 15-150 nucleotides in length.

Embodiment 190: The composition of Embodiment 181, wherein the second editing template is about 15-100 nucleotides in length.

Embodiment 191: The composition of Embodiment 181, wherein the second editing template is about 30-100 nucleotides in length.

Embodiment 192: The composition of Embodiment 181, wherein the second editing template is about 50-100 nucleotides in length.

Embodiment 193: The composition of Embodiment 181, wherein the second editing template is about 15-50 nucleotides in length.

Embodiment 194: The composition of any one of the preceding Embodiments 103-194, wherein the first editing template and second editing template are of the same length.

Embodiment 195: The composition of any one of Embodiments 103-193, wherein the first editing template and second editing template are of different lengths.

Embodiment 196: The composition of any one of the preceding Embodiments 103-196, wherein the first and/or the second spacer is about 15 to 25 nucleotides in length.

Embodiment 197: The composition of Embodiment 196, wherein the first and/or the second spacer is about 17 to 22 nucleotides in length.

Embodiment 198: The composition of Embodiment 196, wherein the first and/or the second spacer is about 20 to 22 nucleotides in length.

Embodiment 200: A dual prime editing system comprising the composition of any one of the preceding Embodiments and further comprising a first prime editor that comprises a DNA binding domain and a DNA polymerase domain and associates with the first PEgRNA, and a second prime editor that comprises a DNA binding domain and a DNA polymerase domain and associates with the second PEgRNA.

Embodiment 201: The dual prime editing system of Embodiment 200, wherein the first prime editor and the second prime editor are the same.

Embodiment 202: The dual prime editing system of Embodiment 201, wherein the DNA binding domain is a CRISPR associated (Cas) protein domain.

Embodiment 203: The dual prime editing system of Embodiment 202, wherein the Cas protein domain has a nickase activity.

Embodiment 204: The dual prime editing system of Embodiment 202, wherein the Cas protein domain is a Cas9.

Embodiment 205: The dual prime editing system of Embodiment 204, wherein the Cas9 comprises a mutation in an HNH domain.

Embodiment 206: The dual prime editing system of Embodiment 204, wherein the Cas9 comprises a H840A mutation in the HNH domain.

Embodiment 207: The dual prime editing system of Embodiment 202, wherein the Cas protein domain is a Cas12b.

Embodiment 208: The dual prime editing system of Embodiment 202, wherein the Cas protein domain is a Cas12a, Cas12b, Cas12c, Cas12d, Cas12e, Cas14a, Cas14b, Cas14c, Cas14d, Cas14e, Cas14f, Cas14g, Cas14h, Cas14u, or a Cas φ.

Embodiment 209: The dual prime editing system of any one of Embodiments 200-208, wherein the DNA polymerase domain is a reverse transcriptase.

Embodiment 210: The dual prime editing system of Embodiment 209, wherein the reverse transcriptase is a retrovirus reverse transcriptase.

Embodiment 211: The dual prime editing system of Embodiment 210, wherein the reverse transcriptase is a Moloney murine leukemia virus (M-MLV) reverse transcriptase.

Embodiment 212: The dual prime editing system of any one of Embodiments 200-211, wherein the DNA polymerase domain and the DNA binding domain are fused or linked to form a fusion protein, and wherein the DNA binding domain is a programmable DNA binding domain.

Embodiment 213: A lipid nanoparticle (LNP) or ribonucleoprotein (RNP) comprising the dual prime editing system of any one of Embodiments 200-212, or a component thereof.

Embodiment 214: A polynucleotide encoding the first PEgRNA and the second PEgRNA of any one of Embodiments 103-198, the dual prime editing system of any one of Embodiments 200-212, or a component thereof.

Embodiment 215: The polynucleotide of Embodiment 214, wherein the polynucleotide is a mRNA.

Embodiment 216: The polynucleotide of Embodiment 214 or 215, wherein the polynucleotide is operably linked to a regulatory element.

Embodiment 217: The polynucleotide of Embodiment 216, wherein the regulatory element is an inducible regulatory element.

Embodiment 218: A vector comprising the polynucleotide of any one of Embodiments 214-217.

Embodiment 219: The vector of Embodiment 218, wherein the vector is an AAV vector.

Embodiment 220: An isolated cell comprising the first PEgRNA and second PEgRNA of any one of Embodiments 103-198, the dual prime editing system of any one of Embodiments 200-212, the LNP or RNP of Embodiment 213, the polynucleotide of any one of Embodiments 214-217, or the vector of Embodiments 218 or 219.

Embodiment 221: The cell of Embodiment 220, wherein the cell is a human cell.

Embodiment 222: The cell of Embodiment 220 or 221, wherein the cell is a fibroblast, a myoblast, a myosatellite, a muscle progenitor cell, a skeletal muscle cell, a smooth muscle cell, or a differentiated muscle cell.

Embodiment 223: A pharmaceutical composition comprising (i) the composition of any one of Embodiments 103-198, the dual prime editing system of any one of Embodiments 200-212, the LNP or RNP of Embodiment 213, the polynucleotide of any one of Embodiments 214-217, or the vector of Embodiments 218 or 219, or the cell of any one of Embodiments 220-222; and (ii) a pharmaceutically acceptable carrier.

Embodiment 224: A method for editing a DMPK gene, the method comprising contacting the DMPK gene with (i) the composition of any one of Embodiments 103-198, (ii) a first prime editor comprising a DNA binding domain and a DNA polymerase domain that associates with the first PEgRNA, and (iii) a second prime editor comprising a DNA binding domain and a DNA polymerase domain that associates with the second PEgRNA, wherein the first PEgRNA directs the first prime editor to generate a first nick on the second strand of the DMPK gene, wherein the second PEgRNA directs the second prime editor to generate a second nick on the first strand of the DMPK gene, and wherein the contacting results in excision of an inter-nick duplex (IND) between the position of the first nick and the position of the second nick of the DMPK gene, thereby editing the DMPK gene.

Embodiment 225: A method for editing a DMPK gene, the method comprising contacting the DMPK gene with the dual prime editing system of any one of Embodiments 200-211, wherein the first PEgRNA directs the first prime editor to generate a first nick on the second strand of the DMPK gene, wherein the second PEgRNA directs the second prime editor to generate a second nick on the first strand of the DMPK gene, and wherein the contacting results in excision of an inter-nick duplex (IND) between the position of the first nick and the position of the second nick of the DMPK gene, thereby editing the DMPK gene.

Embodiment 226: The method of Embodiment 224 or 225, wherein the first prime editor and the second prime editor are the same.

Embodiment 227: The method of any one of Embodiments 224-226, wherein the first editing template encodes a first single stranded DNA, and wherein the first single stranded DNA is incorporated in the DMPK gene.

Embodiment 228: The method of any one of Embodiments 224-227, wherein the second editing template encodes a second single stranded DNA, and wherein the second single stranded DNA is incorporated in the DMPK gene.

Embodiment 229: The method of any one of Embodiments 224-228, wherein the contacting results in deletion of the array of tri-nucleotide repeats in the DMPK gene.

Embodiment 230: The method of any one of Embodiments 224-228, wherein the contacting results in a reduced number of tri-nucleotide repeats in the DMPK gene.

Embodiment 231: The method of any one of Embodiments 224-230, wherein the contacting results in deletion of the sequence (CTG)x in the DMPK gene, wherein x is an integer no less than 1.

Embodiment 232: The method of Embodiment 231, wherein x is an integer between 5 and 30.

Embodiment 233: The method of Embodiment 231, wherein x is an integer greater than 50.

Embodiment 234: The method of Embodiment 231, wherein x is an integer greater than 100.

Embodiment 235: The method of Embodiment 231, wherein x is an integer greater than 1000.

Embodiment 236: The method of any one of Embodiments 224-235, wherein the contacting results in no greater than 33 CTG repeats in a 3′ untranslated region (3′ UTR) of the DMPK gene.

Embodiment 237: The method of Embodiment 236, wherein the contacting results in no CTG repeats in a 3′ untranslated region (3′ UTR) of the DMPK gene.

Embodiment 238: The method of any one of Embodiments 224-237, wherein the DMPK gene is in a cell.

Embodiment 239: The method of Embodiment 238, wherein the cell is a mammalian cell, human cell, primary cell, or muscle cell.

Embodiment 240: The method of Embodiment 238 or 239, wherein the cell is a fibroblast, a myoblast, a myosatellite, a muscle progenitor cell, a skeletal muscle cell, a smooth muscle cell, or a differentiated muscle cell.

Embodiment 241: The method of any one of Embodiments 238-240, wherein the cell is in a subject.

Embodiment 242: The method of Embodiment 241, wherein the subject is a human.

Embodiment 243: The method of any one of Embodiments 238-242, wherein the cell is from a subject having Myotonic dystrophy.

Embodiment 244: The method of Embodiment 243, further comprising administering the cell to the subject after the contacting.

Embodiment 245: A method for treating myotonic dystrophy in a subject in need thereof, the method comprising administering to the subject the composition of any one of Embodiments 103-198, the dual prime editing system of any one of Embodiments 200-212, the LNP or RNP of Embodiment 213, the polynucleotide of any one of Embodiments 214-217, the vector of Embodiments 218 or 219, or the cell of any one of Embodiments 220-222, or the pharmaceutical composition of Embodiment 223, wherein the administration results in a reduced number of an array of CTG repeats in the DMPK gene in the subject, thereby treating myotonic dystrophy in the subject.

Embodiment 246: The method of Embodiment 231, wherein the subject is a human.

### EXAMPLES

The following examples are provided for illustrative purposes only and are not intended to limit the scope of the claims provided herein. For Examples 2-8, the indicated PEgRNA sequence does not contain the adaptations for transcription from a DNA template used experimentally (i.e., addition of a 5′G if the spacer did not already start with a G and addition of 1-6 3′U from the U6 transcription termination sequence).

### Example 1. General Methods

PEgRNA assembly: PEgRNA libraries are assembled by one of three methods: in the first method, pooled synthesized DNA oligos encoding the PEgRNA and flanking U6 expression plasmid homology regions are cloned into U6 expression plasmids via Gibson cloning and sequencing of bacterial colonies via Sanger or Next-generation sequencing. In the second method, double-stranded linear DNA fragments encoding PEgRNA and homology sequences as above are individually Gibson-cloned into U6 expression plasmids. In the third method, for each PEgRNA, separate oligos encoding a protospacer, a gRNA scaffold, and PEgRNA extension (PBS and RTT) are ligated, and then cloned into a U6 expression plasmid as described in Anzalone et al., Nature. 2019 December; 576(7785):149-157. Bacterial colonies carrying sequence-verified plasmids are propagated in TB. Plasmid DNA is purified by minipreps for mammalian transfection.

HEK cell culture and transfection: HEK293T cells are propagated in DMEM with 10% FBS. Prior to transfection, cells are seeded in 96-well plates and then transfected with Lipofectamine 2000 according to the manufacturer's directions with polynucleotide encoding a prime editor fusion protein and a pair of PEgRNAs. Three days after transfection, gDNA is harvested in lysis buffer for high throughput sequencing and is sequenced using Miseq.

iPSC transfection and editing analysis. iPSC cells are plated in 96-well plate on Matrigel-coated plates in E8 media with 10 μM of ROCK inhibitor. Cells are transfected on the following day with Lipofectamine STEM according to the manufacturer's directions with polynucleotides encoding a pair of PEgRNAs and a prime editor fusion protein. Three days after transfection, gDNA is harvested in lysis buffer for sequencing and analysis with droplet digital PCR (ddPCR).

### Example 2. Screening of PEgRNA for Excising CTG Repeats Associated with Myotonic Dystrophy Type I

A PEgRNA library was designed to replace the CTG repeats in the DMPK gene with an exogenous 38 nt attB sequence. Three days after transfection, gDNA was harvested in lysis buffer for high throughput sequencing and sequenced using MiSeq.

Wild type HEK 293T cells were transfected with polynucleotides encoding a prime editor fusion protein a pair of PEgRNAs as described in Example 1. Editing efficiency of dual prime editing is reflected by excision of the 5 CTG repeats in wildtype HEK293T cells and integration of the attB sequence encoded by the RTTs of the PEgRNA pair.

An editing DMPK sequence is expected to contains a 38 nt attB sequence that replaces the CTG repeats. A 72 nt sequence, which contains the 38 nt attB sequence flanked by 17 bp of endogenous, non-CTG repeat DMPK sequence on each side, was used as a marker sequence for attB integration at the position of CTG repeat excision to assess dual prime editing efficiency. Sequencing read numbers, including (a) total editing reads, (b) reads of sequences that have exact match to the 72 bp sequence including the attB sequence and the flanking sequences on both sides, and (c) reads of sequences that have match to the 38 bp attB sequence (but not necessarily match to the 72 bp sequence) were calculated. Editing and indel frequency was quantified using the following formula: editing %=(b)/(a), and indel %=(c)/(a).

The results are shown in Table 51 below. In Table 51, each row presents information for a pair of PEgRNAs (i.e. a 5′ PEgRNA and a 3′ PEgRNA) used in dual prime editing. Each PEgRNA is identified by a sequence identifier (“SEQ ID NO”), and information is also presented for the spacer number and PBS length of each PEgRNA that was tested. The following scale was used for editing efficiency: PEgRNA pairs exhibiting editing efficiency between 0% to 1% are indicated with “+”, PEgRNA pairs exhibiting editing efficiency of 1% to 10% are indicated with “++”, and PEgRNA pairs exhibiting editing efficiency of 10% to 100% are indicated with “+++”.

TABLE 51
Dual prime editing pegRNA screen for CTG excision in HEK 293T cells.

| | 5′ PEgRNA | | | 3′ PEgRNA | 3′ PBS | | |
|---|---|---|---|---|---|---|---|
| 5′ spacer No. | SEQ ID | 5′ PBS length | 3′ spacer | SEQ ID | length | % Indel | % Editing |
| 5′ spacer 1 | 18 | 11 | 3′ spacer 1 | 1376 | 10 | + | − |
| 5′ spacer 1 | 17 | 8 | 3′ spacer 1 | 1376 | 10 | + | − |
| 5′ spacer 1 | 19 | 13 | 3′ spacer 1 | 1376 | 10 | + | + |
| 5′ spacer 1 | 17 | 8 | 3′ spacer 1 | 1375 | 8 | + | − |
| 5′ spacer 1 | 17 | 8 | 3′ spacer 1 | 1377 | 12 | + | − |
| 5′ spacer 1 | 18 | 11 | 3′ spacer 1 | 1375 | 8 | + | + |
| 5′ spacer 1 | 18 | 11 | 3′ spacer 1 | 1377 | 12 | + | + |
| 5′ spacer 1 | 19 | 13 | 3′ spacer 1 | 1375 | 8 | + | + |
| 5′ spacer 1 | 19 | 13 | 3′ spacer 1 | 1377 | 12 | + | − |
| 5′ spacer 1 | 17 | 8 | 3′ spacer 2 | 1397 | 8 | + | − |
| 5′ spacer 1 | 17 | 8 | 3′ spacer 2 | 1398 | 10 | + | + |
| 5′ spacer 1 | 17 | 8 | 3′ spacer 2 | 1399 | 12 | + | − |
| 5′ spacer 1 | 18 | 11 | 3′ spacer 2 | 1397 | 8 | + | + |
| 5′ spacer 1 | 19 | 13 | 3′ spacer 2 | 1399 | 12 | + | + |
| 5′ spacer 1 | 19 | 13 | 3′ spacer 2 | 1397 | 8 | + | − |
| 5′ spacer 1 | 19 | 13 | 3′ spacer 2 | 1398 | 10 | + | + |
| 5′ spacer 1 | 18 | 11 | 3′ spacer 2 | 1399 | 12 | + | + |
| 5′ spacer 1 | 18 | 11 | 3′ spacer 2 | 1398 | 10 | + | + |
| 5′ spacer 1 | 18 | 11 | 3′ spacer 3 | 1420 | 10 | +++ | − |
| 5′ spacer 1 | 17 | 8 | 3′ spacer 3 | 1419 | 8 | +++ | − |
| 5′ spacer 1 | 19 | 13 | 3′ spacer 3 | 1419 | 8 | +++ | − |
| 5′ spacer 1 | 17 | 8 | 3′ spacer 3 | 1421 | 14 | +++ | − |
| 5′ spacer 1 | 19 | 13 | 3′ spacer 3 | 1420 | 10 | +++ | + |
| 5′ spacer 1 | 19 | 13 | 3′ spacer 3 | 1421 | 14 | +++ | + |
| 5′ spacer 1 | 18 | 11 | 3′ spacer 3 | 1419 | 8 | +++ | − |
| 5′ spacer 1 | 18 | 11 | 3′ spacer 3 | 1421 | 14 | +++ | + |
| 5′ spacer 1 | 17 | 8 | 3′ spacer 3 | 1420 | 10 | +++ | − |
| 5′ spacer 1 | 17 | 8 | 3′ spacer 4 | 1521 | 12 | ++ | + |
| 5′ spacer 1 | 18 | 11 | 3′ spacer 4 | 1519 | 8 | + | − |
| 5′ spacer 1 | 18 | 11 | 3′ spacer 4 | 1521 | 12 | + | − |
| 5′ spacer 1 | 19 | 13 | 3′ spacer 4 | 1520 | 10 | ++ | − |
| 5′ spacer 1 | 19 | 13 | 3′ spacer 4 | 1519 | 8 | + | − |
| 5′ spacer 1 | 18 | 11 | 3′ spacer 4 | 1520 | 10 | +++ | − |
| 5′ spacer 1 | 17 | 8 | 3′ spacer 4 | 1520 | 10 | +++ | − |
| 5′ spacer 1 | 17 | 8 | 3′ spacer 4 | 1519 | 8 | + | − |
| 5′ spacer 1 | 19 | 13 | 3′ spacer 4 | 1521 | 12 | + | − |
| 5′ spacer 1 | 19 | 13 | 3′ spacer 5 | 1543 | 12 | + | + |
| 5′ spacer 1 | 17 | 8 | 3′ spacer 5 | 1543 | 12 | + | − |
| 5′ spacer 1 | 18 | 11 | 3′ spacer 5 | 1541 | 8 | + | + |
| 5′ spacer 1 | 17 | 8 | 3′ spacer 5 | 1541 | 8 | ++ | − |
| 5′ spacer 1 | 19 | 13 | 3′ spacer 5 | 1541 | 8 | + | + |
| 5′ spacer 1 | 18 | 11 | 3′ spacer 5 | 1543 | 12 | + | + |
| 5′ spacer 1 | 19 | 13 | 3′ spacer 5 | 1542 | 10 | + | + |
| 5′ spacer 1 | 18 | 11 | 3′ spacer 5 | 1542 | 10 | + | + |
| 5′ spacer 1 | 17 | 8 | 3′ spacer 5 | 1542 | 10 | + | + |
| 5′ spacer 3 | 57 | 12 | 3′ spacer 1 | 1375 | 8 | + | + |
| 5′ spacer 3 | 57 | 12 | 3′ spacer 1 | 1377 | 12 | + | + |
| 5′ spacer 3 | 55 | 8 | 3′ spacer 1 | 1377 | 12 | + | + |
| 5′ spacer 3 | 56 | 10 | 3′ spacer 1 | 1377 | 12 | + | + |
| 5′ spacer 3 | 55 | 8 | 3′ spacer 1 | 1375 | 8 | + | + |
| 5′ spacer 3 | 56 | 10 | 3′ spacer 1 | 1376 | 10 | + | + |
| 5′ spacer 3 | 57 | 12 | 3′ spacer 1 | 1376 | 10 | + | + |
| 5′ spacer 3 | 56 | 10 | 3′ spacer 1 | 1375 | 8 | + | + |
| 5′ spacer 3 | 55 | 8 | 3′ spacer 1 | 1376 | 10 | + | + |
| 5′ spacer 3 | 56 | 10 | 3′ spacer 2 | 1398 | 10 | + | + |
| 5′ spacer 3 | 56 | 10 | 3′ spacer 2 | 1397 | 8 | + | + |
| 5′ spacer 3 | 56 | 10 | 3′ spacer 2 | 1399 | 12 | + | + |
| 5′ spacer 3 | 55 | 8 | 3′ spacer 2 | 1399 | 12 | + | + |
| 5′ spacer 3 | 57 | 12 | 3′ spacer 2 | 1399 | 12 | + | + |
| 5′ spacer 3 | 57 | 12 | 3′ spacer 2 | 1398 | 10 | + | + |
| 5′ spacer 3 | 57 | 12 | 3′ spacer 2 | 1397 | 8 | + | + |
| 5′ spacer 3 | 55 | 8 | 3′ spacer 2 | 1398 | 10 | + | + |
| 5′ spacer 3 | 55 | 8 | 3′ spacer 2 | 1397 | 8 | + | + |
| 5′ spacer 3 | 55 | 8 | 3′ spacer 3 | 1419 | 8 | +++ | + |
| 5′ spacer 3 | 55 | 8 | 3′ spacer 3 | 1421 | 14 | +++ | + |
| 5′ spacer 3 | 57 | 12 | 3′ spacer 3 | 1420 | 10 | +++ | + |
| 5′ spacer 3 | 56 | 10 | 3′ spacer 3 | 1421 | 14 | +++ | + |
| 5′ spacer 3 | 57 | 12 | 3′ spacer 3 | 1419 | 8 | +++ | + |
| 5′ spacer 3 | 55 | 8 | 3′ spacer 3 | 1420 | 10 | ++ | + |
| 5′ spacer 3 | 57 | 12 | 3′ spacer 3 | 1421 | 14 | +++ | + |
| 5′ spacer 3 | 56 | 10 | 3′ spacer 3 | 1419 | 8 | +++ | + |
| 5′ spacer 3 | 56 | 10 | 3′ spacer 3 | 1420 | 10 | +++ | + |
| 5′ spacer 3 | 56 | 10 | 3′ spacer 4 | 1521 | 12 | + | + |
| 5′ spacer 3 | 57 | 12 | 3′ spacer 4 | 1520 | 10 | +++ | + |
| 5′ spacer 3 | 55 | 8 | 3′ spacer 4 | 1519 | 8 | + | + |
| 5′ spacer 3 | 55 | 8 | 3′ spacer 4 | 1520 | 10 | +++ | + |
| 5′ spacer 3 | 57 | 12 | 3′ spacer 4 | 1521 | 12 | + | + |
| 5′ spacer 3 | 56 | 10 | 3′ spacer 4 | 1520 | 10 | +++ | + |
| 5′ spacer 3 | 55 | 8 | 3′ spacer 4 | 1521 | 12 | + | + |
| 5′ spacer 3 | 57 | 12 | 3′ spacer 4 | 1519 | 8 | + | − |
| 5′ spacer 3 | 56 | 10 | 3′ spacer 4 | 1519 | 8 | + | − |
| 5′ spacer 3 | 56 | 10 | 3′ spacer 5 | 1542 | 10 | + | + |
| 5′ spacer 3 | 56 | 10 | 3′ spacer 5 | 1543 | 12 | + | + |
| 5′ spacer 3 | 55 | 8 | 3′ spacer 5 | 1542 | 10 | + | + |
| 5′ spacer 3 | 55 | 8 | 3′ spacer 5 | 1543 | 12 | + | + |
| 5′ spacer 3 | 57 | 12 | 3′ spacer 5 | 1541 | 8 | + | + |
| 5′ spacer 3 | 56 | 10 | 3′ spacer 5 | 1541 | 8 | + | + |
| 5′ spacer 3 | 57 | 12 | 3′ spacer 5 | 1542 | 10 | + | + |
| 5′ spacer 3 | 57 | 12 | 3′ spacer 5 | 1543 | 12 | + | + |
| 5′ spacer 3 | 55 | 8 | 3′ spacer 5 | 1541 | 8 | + | + |
| 5′ spacer 4 | 74 | 8 | 3′ spacer 1 | 1376 | 10 | + | ++ |
| 5′ spacer 4 | 75 | 10 | 3′ spacer 1 | 1377 | 12 | + | + |
| 5′ spacer 4 | 75 | 10 | 3′ spacer 1 | 1375 | 8 | +++ | + |
| 5′ spacer 4 | 76 | 12 | 3′ spacer 1 | 1377 | 12 | + | ++ |
| 5′ spacer 4 | 75 | 10 | 3′ spacer 1 | 1376 | 10 | + | + |
| 5′ spacer 4 | 74 | 8 | 3′ spacer 1 | 1375 | 8 | ++ | + |
| 5′ spacer 4 | 74 | 8 | 3′ spacer 1 | 1377 | 12 | + | ++ |
| 5′ spacer 4 | 76 | 12 | 3′ spacer 1 | 1375 | 8 | ++ | + |
| 5′ spacer 4 | 76 | 12 | 3′ spacer 1 | 1376 | 10 | + | ++ |
| 5′ spacer 4 | 76 | 12 | 3′ spacer 2 | 1397 | 8 | + | ++ |
| 5′ spacer 4 | 75 | 10 | 3′ spacer 2 | 1399 | 12 | + | ++ |
| 5′ spacer 4 | 76 | 12 | 3′ spacer 2 | 1398 | 10 | + | ++ |
| 5′ spacer 4 | 75 | 10 | 3′ spacer 2 | 1397 | 8 | + | ++ |
| 5′ spacer 4 | 76 | 12 | 3′ spacer 2 | 1399 | 12 | + | ++ |
| 5′ spacer 4 | 74 | 8 | 3′ spacer 2 | 1397 | 8 | + | ++ |
| 5′ spacer 4 | 74 | 8 | 3′ spacer 2 | 1399 | 12 | + | ++ |
| 5′ spacer 4 | 75 | 10 | 3′ spacer 2 | 1398 | 10 | + | ++ |
| 5′ spacer 4 | 74 | 8 | 3′ spacer 2 | 1398 | 10 | + | ++ |
| 5′ spacer 4 | 76 | 12 | 3′ spacer 3 | 1420 | 10 | ++ | +++ |
| 5′ spacer 4 | 75 | 10 | 3′ spacer 3 | 1419 | 8 | ++ | ++ |
| 5′ spacer 4 | 76 | 12 | 3′ spacer 3 | 1419 | 8 | ++ | ++ |
| 5′ spacer 4 | 74 | 8 | 3′ spacer 3 | 1420 | 10 | ++ | ++ |
| 5′ spacer 4 | 76 | 12 | 3′ spacer 3 | 1421 | 14 | ++ | ++ |
| 5′ spacer 4 | 74 | 8 | 3′ spacer 3 | 1421 | 14 | +++ | ++ |
| 5′ spacer 4 | 74 | 8 | 3′ spacer 3 | 1419 | 8 | ++ | ++ |
| 5′ spacer 4 | 75 | 10 | 3′ spacer 3 | 1421 | 14 | +++ | ++ |
| 5′ spacer 4 | 75 | 10 | 3′ spacer 3 | 1420 | 10 | +++ | ++ |
| 5′ spacer 4 | 74 | 8 | 3′ spacer 4 | 1519 | 8 | + | + |
| 5′ spacer 4 | 75 | 10 | 3′ spacer 4 | 1520 | 10 | +++ | + |
| 5′ spacer 4 | 75 | 10 | 3′ spacer 4 | 1519 | 8 | + | + |
| 5′ spacer 4 | 75 | 10 | 3′ spacer 4 | 1521 | 12 | ++ | ++ |
| 5′ spacer 4 | 74 | 8 | 3′ spacer 4 | 1521 | 12 | ++ | ++ |
| 5′ spacer 4 | 76 | 12 | 3′ spacer 4 | 1520 | 10 | ++ | + |
| 5′ spacer 4 | 76 | 12 | 3′ spacer 4 | 1521 | 12 | + | ++ |
| 5′ spacer 4 | 74 | 8 | 3′ spacer 4 | 1520 | 10 | ++ | + |
| 5′ spacer 4 | 76 | 12 | 3′ spacer 4 | 1519 | 8 | + | + |
| 5′ spacer 4 | 76 | 12 | 3′ spacer 5 | 1542 | 10 | ++ | +++ |
| 5′ spacer 4 | 76 | 12 | 3′ spacer 5 | 1543 | 12 | +++ | +++ |
| 5′ spacer 4 | 75 | 10 | 3′ spacer 5 | 1543 | 12 | +++ | +++ |
| 5′ spacer 4 | 74 | 8 | 3′ spacer 5 | 1542 | 10 | +++ | +++ |
| 5′ spacer 4 | 76 | 12 | 3′ spacer 5 | 1541 | 8 | +++ | +++ |
| 5′ spacer 4 | 74 | 8 | 3′ spacer 5 | 1541 | 8 | +++ | +++ |
| 5′ spacer 4 | 75 | 10 | 3′ spacer 5 | 1542 | 10 | +++ | +++ |
| 5′ spacer 4 | 74 | 8 | 3′ spacer 5 | 1543 | 12 | +++ | +++ |
| 5′ spacer 4 | 75 | 10 | 3′ spacer 5 | 1541 | 8 | +++ | +++ |
| 5′ spacer 5 | 94 | 10 | 3′ spacer 1 | 1377 | 12 | ++ | ++ |
| 5′ spacer 5 | 93 | 8 | 3′ spacer 1 | 1375 | 8 | +++ | ++ |
| 5′ spacer 5 | 95 | 13 | 3′ spacer 1 | 1375 | 8 | +++ | + |
| 5′ spacer 5 | 95 | 13 | 3′ spacer 1 | 1377 | 12 | ++ | ++ |
| 5′ spacer 5 | 94 | 10 | 3′ spacer 1 | 1376 | 10 | ++ | ++ |
| 5′ spacer 5 | 93 | 8 | 3′ spacer 1 | 1377 | 12 | ++ | ++ |
| 5′ spacer 5 | 94 | 10 | 3′ spacer 1 | 1375 | 8 | +++ | ++ |
| 5′ spacer 5 | 95 | 13 | 3′ spacer 1 | 1376 | 10 | ++ | ++ |
| 5′ spacer 5 | 93 | 8 | 3′ spacer 1 | 1376 | 10 | ++ | +++ |
| 5′ spacer 5 | 93 | 8 | 3′ spacer 2 | 1399 | 12 | ++ | +++ |
| 5′ spacer 5 | 95 | 13 | 3′ spacer 2 | 1397 | 8 | ++ | ++ |
| 5′ spacer 5 | 93 | 8 | 3′ spacer 2 | 1397 | 8 | ++ | +++ |
| 5′ spacer 5 | 94 | 10 | 3′ spacer 2 | 1397 | 8 | ++ | +++ |
| 5′ spacer 5 | 93 | 8 | 3′ spacer 2 | 1398 | 10 | ++ | +++ |
| 5′ spacer 5 | 95 | 13 | 3′ spacer 2 | 1398 | 10 | +++ | +++ |
| 5′ spacer 5 | 94 | 10 | 3′ spacer 2 | 1399 | 12 | ++ | +++ |
| 5′ spacer 5 | 95 | 13 | 3′ spacer 2 | 1399 | 12 | ++ | +++ |
| 5′ spacer 5 | 94 | 10 | 3′ spacer 2 | 1398 | 10 | ++ | +++ |
| 5′ spacer 5 | 94 | 10 | 3′ spacer 3 | 1419 | 8 | ++ | ++ |
| 5′ spacer 5 | 93 | 8 | 3′ spacer 3 | 1420 | 10 | +++ | +++ |
| 5′ spacer 5 | 95 | 13 | 3′ spacer 3 | 1419 | 8 | ++ | ++ |
| 5′ spacer 5 | 95 | 13 | 3′ spacer 3 | 1421 | 14 | +++ | +++ |
| 5′ spacer 5 | 93 | 8 | 3′ spacer 3 | 1419 | 8 | ++ | ++ |
| 5′ spacer 5 | 94 | 10 | 3′ spacer 3 | 1421 | 14 | +++ | +++ |
| 5′ spacer 5 | 93 | 8 | 3′ spacer 3 | 1421 | 14 | +++ | +++ |
| 5′ spacer 5 | 94 | 10 | 3′ spacer 3 | 1420 | 10 | +++ | +++ |
| 5′ spacer 5 | 95 | 13 | 3′ spacer 3 | 1420 | 10 | +++ | +++ |
| 5′ spacer 5 | 94 | 10 | 3′ spacer 4 | 1519 | 8 | + | + |
| 5′ spacer 5 | 93 | 8 | 3′ spacer 4 | 1521 | 12 | + | ++ |
| 5′ spacer 5 | 94 | 10 | 3′ spacer 4 | 1520 | 10 | ++ | ++ |
| 5′ spacer 5 | 93 | 8 | 3′ spacer 4 | 1519 | 8 | + | + |
| 5′ spacer 5 | 93 | 8 | 3′ spacer 4 | 1520 | 10 | +++ | ++ |
| 5′ spacer 5 | 95 | 13 | 3′ spacer 4 | 1520 | 10 | ++ | ++ |
| 5′ spacer 5 | 95 | 13 | 3′ spacer 4 | 1521 | 12 | ++ | ++ |
| 5′ spacer 5 | 95 | 13 | 3′ spacer 4 | 1519 | 8 | ++ | + |
| 5′ spacer 5 | 94 | 10 | 3′ spacer 4 | 1521 | 12 | ++ | ++ |
| 5′ spacer 5 | 95 | 13 | 3′ spacer 5 | 1542 | 10 | ++ | +++ |
| 5′ spacer 5 | 93 | 8 | 3′ spacer 5 | 1541 | 8 | ++ | +++ |
| 5′ spacer 5 | 94 | 10 | 3′ spacer 5 | 1542 | 10 | ++ | +++ |
| 5′ spacer 5 | 95 | 13 | 3′ spacer 5 | 1541 | 8 | ++ | +++ |
| 5′ spacer 5 | 94 | 10 | 3′ spacer 5 | 1543 | 12 | ++ | +++ |
| 5′ spacer 5 | 95 | 13 | 3′ spacer 5 | 1543 | 12 | ++ | +++ |
| 5′ spacer 5 | 93 | 8 | 3′ spacer 5 | 1542 | 10 | ++ | +++ |
| 5′ spacer 5 | 93 | 8 | 3′ spacer 5 | 1543 | 12 | ++ | +++ |
| 5′ spacer 5 | 94 | 10 | 3′ spacer 5 | 1541 | 8 | ++ | +++ |
| 5′ spacer 6 | 500 | 8 | 3′ spacer 1 | 1377 | 12 | + | ++ |
| 5′ spacer 6 | 502 | 12 | 3′ spacer 1 | 1375 | 8 | +++ | + |
| 5′ spacer 6 | 502 | 12 | 3′ spacer 1 | 1376 | 10 | ++ | ++ |
| 5′ spacer 6 | 501 | 10 | 3′ spacer 1 | 1376 | 10 | ++ | ++ |
| 5′ spacer 6 | 500 | 8 | 3′ spacer 1 | 1375 | 8 | +++ | + |
| 5′ spacer 6 | 500 | 8 | 3′ spacer 1 | 1376 | 10 | + | ++ |
| 5′ spacer 6 | 501 | 10 | 3′ spacer 1 | 1377 | 12 | ++ | ++ |
| 5′ spacer 6 | 502 | 12 | 3′ spacer 1 | 1377 | 12 | +++ | + |
| 5′ spacer 6 | 501 | 10 | 3′ spacer 1 | 1375 | 8 | +++ | ++ |
| 5′ spacer 6 | 501 | 10 | 3′ spacer 2 | 1397 | 8 | + | +++ |
| 5′ spacer 6 | 502 | 12 | 3′ spacer 2 | 1397 | 8 | + | + |
| 5′ spacer 6 | 500 | 8 | 3′ spacer 2 | 1399 | 12 | + | +++ |
| 5′ spacer 6 | 502 | 12 | 3′ spacer 2 | 1399 | 12 | + | +++ |
| 5′ spacer 6 | 502 | 12 | 3′ spacer 2 | 1398 | 10 | + | +++ |
| 5′ spacer 6 | 500 | 8 | 3′ spacer 2 | 1397 | 8 | + | ++ |
| 5′ spacer 6 | 501 | 10 | 3′ spacer 2 | 1398 | 10 | + | +++ |
| 5′ spacer 6 | 500 | 8 | 3′ spacer 2 | 1398 | 10 | + | +++ |
| 5′ spacer 6 | 501 | 10 | 3′ spacer 2 | 1399 | 12 | + | +++ |
| 5′ spacer 6 | 500 | 8 | 3′ spacer 3 | 1421 | 14 | +++ | +++ |
| 5′ spacer 6 | 501 | 10 | 3′ spacer 3 | 1421 | 14 | +++ | +++ |
| 5′ spacer 6 | 502 | 12 | 3′ spacer 3 | 1421 | 14 | +++ | +++ |
| 5′ spacer 6 | 501 | 10 | 3′ spacer 3 | 1419 | 8 | +++ | ++ |
| 5′ spacer 6 | 500 | 8 | 3′ spacer 3 | 1419 | 8 | ++ | ++ |
| 5′ spacer 6 | 502 | 12 | 3′ spacer 3 | 1419 | 8 | ++ | ++ |
| 5′ spacer 6 | 500 | 8 | 3′ spacer 3 | 1420 | 10 | +++ | +++ |
| 5′ spacer 6 | 501 | 10 | 3′ spacer 3 | 1420 | 10 | +++ | +++ |
| 5′ spacer 6 | 502 | 12 | 3′ spacer 3 | 1420 | 10 | +++ | +++ |
| 5′ spacer 6 | 500 | 8 | 3′ spacer 4 | 1521 | 12 | + | ++ |
| 5′ spacer 6 | 500 | 8 | 3′ spacer 4 | 1519 | 8 | + | + |
| 5′ spacer 6 | 500 | 8 | 3′ spacer 4 | 1520 | 10 | ++ | ++ |
| 5′ spacer 6 | 502 | 12 | 3′ spacer 4 | 1520 | 10 | + | ++ |
| 5′ spacer 6 | 501 | 10 | 3′ spacer 4 | 1519 | 8 | + | + |
| 5′ spacer 6 | 502 | 12 | 3′ spacer 4 | 1521 | 12 | + | ++ |
| 5′ spacer 6 | 502 | 12 | 3′ spacer 4 | 1519 | 8 | + | + |
| 5′ spacer 6 | 501 | 10 | 3′ spacer 4 | 1521 | 12 | ++ | +++ |
| 5′ spacer 6 | 501 | 10 | 3′ spacer 4 | 1520 | 10 | + | ++ |
| 5′ spacer 6 | 502 | 12 | 3′ spacer 5 | 1542 | 10 | + | +++ |
| 5′ spacer 6 | 500 | 8 | 3′ spacer 5 | 1541 | 8 | + | +++ |
| 5′ spacer 6 | 500 | 8 | 3′ spacer 5 | 1543 | 12 | + | +++ |
| 5′ spacer 6 | 502 | 12 | 3′ spacer 5 | 1541 | 8 | + | +++ |
| 5′ spacer 6 | 501 | 10 | 3′ spacer 5 | 1541 | 8 | + | +++ |
| 5′ spacer 6 | 502 | 12 | 3′ spacer 5 | 1543 | 12 | + | +++ |
| 5′ spacer 6 | 501 | 10 | 3′ spacer 5 | 1542 | 10 | + | +++ |
| 5′ spacer 6 | 501 | 10 | 3′ spacer 5 | 1543 | 12 | + | +++ |
| 5′ spacer 6 | 500 | 8 | 3′ spacer 5 | 1542 | 10 | + | +++ |

A total of fifteen (15) 5′ PEgRNAs and fifteen (15) 3′ PEgRNAs (total 225 PEgRNA combinations) were screened. The 5′ PEgRNA spacers that were screened were 5′ spacers 1 and 3-6 (SEQ ID NOs:1, 39, 58, 77, and 484, respectively). The 3′ PEgRNA spacers that were screened were 3′ spacers 1-5 (SEQ ID NOs: 1359, 1381, 1403, 1503, and 1525, respectively). The 5′ PEgRNA and 3′ PEgRNA PBS lengths were 8, 10, 11, 12 or 13 nucleotides as indicated in Table 51. For each spacer, successful dual prime editing was observed in a portion of the PEgRNAs examined. Specifically, efficient editing was observed for 5′ PEgRNAs having 5′ spacer No. 5 and for 3′ PEgRNAs having 3′ spacer No. 5.

### Example 3. Screening of PEgRNA for Excising CTG Repeats Associated with Myotonic Dystrophy Type I (DM1)

A PEgRNA library was designed to replace the CTG repeats in the DMPK gene with an exogenous 38 nt attB sequence. PEgRNAs were assembled as described in Example 1. Wild type HEK 293T cells were transfected with polynucleotides encoding a prime editor fusion protein and a pair of PEgRNAs in arrayed 96-well plates for assessment of editing by high-throughput sequencing. Three days after transfection, gDNA was harvested in lysis buffer for high throughput sequencing and sequenced using MiSeq.

Editing efficiency of dual prime editing is reflected by excision of the 5 CTG repeats in wildtype HEK293T cells and integration of the attB sequence encoded by the RTTs of the PEgRNA pair. The editing efficiency and indel frequency in HEK293T cells were assessed as follows: Editing % and indel % were determined by version 1 CRISPResso2 dual-flap analysis pipeline. Reads were mapped to both a wild type and an expected theoretical edited reference sequence to interpolate editing efficiency (“% editing efficiency”). Three values were computed for indel rates: (a) forward spacer indels (“% indel_Forward”), (b) reverse spacer indels (“% indel_Reverse”), and (c) inter-nick indels (“% indel_nick_to_nick”). For (a) and (b), a +/−1 bp window around the spacer nick site was used for calculating indels. For (c), the window between the forward spacer nick to the reverse spacer nick site were used for indel calculation. The window size in (c) depends on the spacer pair tested.

The results are summarized in Table 52. In Table 52, each row presents information for a pair of PEgRNAs (i.e., a 5′ PEgRNA and a 3′ PEgRNA) used in dual prime editing. Each PEgRNA is identified by a sequence identifier (“SEQ ID NO”), and information is also presented for the spacer number and PBS length of each PEgRNA that was tested. The following scale was used for editing efficiency: PEgRNA pairs exhibiting editing efficiency above 0% and up to 25% are indicated with “+”, PEgRNA pairs exhibiting editing efficiency above 25% and up to 50% are indicated with “++”, PEgRNA pairs exhibiting editing efficiency above 50% and up to 75% are indicated with “+++”, and PEgRNA pairs exhibiting editing efficiency above 75% and up to 100% are indicated with “++++”. The following scale was used for indel rates: PEgRNA pairs exhibiting indel rates above 0% and up to 7.5% are indicated with “+”, PEgRNA pairs exhibiting indel rates above 7.5% and up to 15% are indicated with “++”, PEgRNA pairs exhibiting indel rates above 15% and up to 22.5% are indicated with “+++”, PEgRNA pairs exhibiting indel rates above 22.5% are indicated with “++++”. “NA” indicates there were insufficient reads for sequencing.

TABLE 52
Dual prime editing pegRNA screen for CTG excision in HEK 293T cells.

| | 5′ PEgRNA | 5′ PBS | | 3′ PEgRNA | 3′ PBS | % editing | % Indel | % Indel | % Indel |
|---|---|---|---|---|---|---|---|---|---|
| 5′ spacer | SEQ ID NO: | length | 3′ spacer | SEQ ID NO: | length | efficiency | Forward | Reverse | Nick to nick |
| 5′ spacer 10 | 588 | 10 | 3′ spacer 9 | 1934 | 10 | ++++ | + | + | + |
| 5′ spacer 10 | 588 | 10 | 3′ spacer 9 | 1935 | 12 | ++++ | NA | + | + |
| 5′ spacer 10 | 588 | 10 | 3′ spacer 10 | 1952 | 10 | ++++ | + | + | + |
| 5′ spacer 10 | 588 | 10 | 3′ spacer 10 | 1953 | 12 | ++++ | + | + | + |
| 5′ spacer 10 | 588 | 10 | 3′ spacer 11 | 2279 | 10 | +++ | + | + | +++ |
| 5′ spacer 10 | 588 | 10 | 3′ spacer 11 | 2280 | 12 | ++++ | + | + | +++ |
| 5′ spacer 10 | 588 | 10 | 3′ spacer 16 | 2369 | 10 | ++++ | + | + | ++ |
| 5′ spacer 10 | 588 | 10 | 3′ spacer 12 | 2297 | 10 | ++++ | + | + | ++ |
| 5′ spacer 10 | 588 | 10 | 3′ spacer 12 | 2298 | 12 | ++++ | + | + | ++ |
| 5′ spacer 10 | 588 | 10 | 3′ spacer 17 | 2689 | 10 | ++++ | + | + | +++ |
| 5′ spacer 10 | 588 | 10 | 3′ spacer 17 | 2690 | 12 | ++++ | + | + | ++ |
| 5′ spacer 10 | 588 | 10 | 3′ spacer 5 | 1543 | 12 | ++++ | + | NA | ++ |
| 5′ spacer 10 | 588 | 10 | 3′ spacer 13 | 2315 | 10 | +++ | + | NA | +++ |
| 5′ spacer 10 | 588 | 10 | 3′ spacer 13 | 2316 | 12 | ++++ | + | NA | +++ |
| 5′ spacer 10 | 588 | 10 | 3′ spacer 14 | 2333 | 10 | ++++ | + | NA | ++ |
| 5′ spacer 10 | 588 | 10 | 3′ spacer 14 | 2334 | 12 | ++++ | + | NA | ++ |
| 5′ spacer 10 | 588 | 10 | 3′ spacer 6 | 1880 | 11 | ++++ | + | + | + |
| 5′ spacer 10 | 588 | 10 | 3′ spacer 6 | 1881 | 13 | ++++ | + | + | + |
| 5′ spacer 10 | 588 | 10 | 3′ spacer 15 | 2352 | 12 | ++++ | + | + | + |
| 5′ spacer 10 | 589 | 13 | 3′ spacer 10 | 1952 | 10 | ++++ | + | + | + |
| 5′ spacer 10 | 589 | 13 | 3′ spacer 10 | 1953 | 12 | ++++ | + | + | + |
| 5′ spacer 10 | 589 | 13 | 3′ spacer 11 | 2279 | 10 | ++++ | + | + | +++ |
| 5′ spacer 10 | 589 | 13 | 3′ spacer 11 | 2280 | 12 | ++++ | + | + | +++ |
| 5′ spacer 10 | 589 | 13 | 3′ spacer 16 | 2369 | 10 | ++++ | + | + | ++ |
| 5′ spacer 10 | 589 | 13 | 3′ spacer 12 | 2297 | 10 | ++++ | + | + | ++ |
| 5′ spacer 10 | 589 | 13 | 3′ spacer 12 | 2298 | 12 | ++++ | + | + | ++ |
| 5′ spacer 10 | 589 | 13 | 3′ spacer 17 | 2689 | 10 | ++++ | + | + | +++ |
| 5′ spacer 10 | 589 | 13 | 3′ spacer 17 | 2690 | 12 | ++++ | + | + | +++ |
| 5′ spacer 10 | 589 | 13 | 3′ spacer 5 | 1543 | 12 | ++++ | + | NA | ++ |
| 5′ spacer 10 | 589 | 13 | 3′ spacer 13 | 2315 | 10 | ++++ | + | NA | +++ |
| 5′ spacer 10 | 589 | 13 | 3′ spacer 13 | 2316 | 12 | +++ | + | NA | ++ |
| 5′ spacer 10 | 589 | 13 | 3′ spacer 14 | 2333 | 10 | ++++ | + | NA | ++ |
| 5′ spacer 10 | 589 | 13 | 3′ spacer 14 | 2334 | 12 | ++++ | + | NA | ++ |
| 5′ spacer 10 | 589 | 13 | 3′ spacer 15 | 2352 | 12 | ++++ | + | + | + |
| 5′ spacer 11 | 606 | 10 | 3′ spacer 8 | 1916 | 10 | + | + | + | + |
| 5′ spacer 11 | 606 | 10 | 3′ spacer 9 | 1934 | 10 | ++++ | + | + | + |
| 5′ spacer 11 | 606 | 10 | 3′ spacer 9 | 1935 | 12 | ++++ | + | + | + |
| 5′ spacer 11 | 606 | 10 | 3′ spacer 10 | 1952 | 10 | ++++ | + | + | + |
| 5′ spacer 11 | 606 | 10 | 3′ spacer 10 | 1953 | 12 | ++++ | + | + | + |
| 5′ spacer 11 | 606 | 10 | 3′ spacer 11 | 2279 | 10 | ++ | + | + | + |
| 5′ spacer 11 | 606 | 10 | 3′ spacer 11 | 2280 | 12 | +++ | + | + | + |
| 5′ spacer 11 | 606 | 10 | 3′ spacer 16 | 2369 | 10 | ++++ | + | + | ++ |
| 5′ spacer 11 | 606 | 10 | 3′ spacer 12 | 2297 | 10 | +++ | + | + | +++ |
| 5′ spacer 11 | 606 | 10 | 3′ spacer 12 | 2298 | 12 | +++ | + | + | ++ |
| 5′ spacer 11 | 606 | 10 | 3′ spacer 17 | 2689 | 10 | +++ | + | + | ++ |
| 5′ spacer 11 | 606 | 10 | 3′ spacer 17 | 2690 | 12 | ++++ | + | + | ++ |
| 5′ spacer 11 | 606 | 10 | 3′ spacer 5 | 1543 | 12 | + | + | + | ++ |
| 5′ spacer 11 | 60€ | 10 | 3′ spacer 13 | 2315 | 10 | +++ | + | + | +++ |
| 5′ spacer 11 | 606 | 10 | 3′ spacer 13 | 2316 | 12 | +++ | + | + | +++ |
| 5′ spacer 11 | 606 | 10 | 3′ spacer 14 | 2333 | 10 | +++ | + | NA | +++ |
| 5′ spacer 11 | 606 | 10 | 3′ spacer 14 | 2334 | 12 | +++ | + | NA | +++ |
| 5′ spacer 11 | 606 | 10 | 3′ spacer 6 | 1880 | 11 | +++ | + | + | + |
| 5′ spacer 11 | 606 | 10 | 3′ spacer 6 | 1881 | 13 | ++++ | + | + | + |
| 5′ spacer 11 | 606 | 10 | 3′ spacer 15 | 2352 | 12 | ++ | + | + | + |
| 5′ spacer 11 | 606 | 10 | 3′ spacer 7 | 1898 | 11 | ++++ | + | + | + |
| 5′ spacer 11 | 606 | 10 | 3′ spacer 7 | 1899 | 13 | ++++ | + | + | + |
| 5′ spacer 11 | 606 | 10 | 3′ spacer 2 | 1399 | 12 | + | + | + | ++ |
| 5′ spacer 11 | 607 | 12 | 3′ spacer 8 | 1916 | 10 | + | + | + | + |
| 5′ spacer 11 | 607 | 12 | 3′ spacer 9 | 1934 | 10 | ++++ | + | + | + |
| 5′ spacer 11 | 607 | 12 | 3′ spacer 9 | 1935 | 12 | ++++ | + | + | + |
| 5′ spacer 11 | 607 | 12 | 3′ spacer 11 | 2279 | 10 | ++ | + | + | + |
| 5′ spacer 11 | 607 | 12 | 3′ spacer 11 | 2280 | 12 | ++++ | + | + | + |
| 5′ spacer 11 | 607 | 12 | 3′ spacer 16 | 2369 | 10 | ++++ | + | + | ++ |
| 5′ spacer 11 | 607 | 12 | 3′ spacer 12 | 2297 | 10 | +++ | + | + | ++ |
| 5′ spacer 11 | 607 | 12 | 3′ spacer 12 | 2298 | 12 | ++ | NA | + | +++ |
| 5′ spacer 11 | 607 | 12 | 3′ spacer 17 | 2689 | 10 | +++ | + | + | ++ |
| 5′ spacer 11 | 607 | 12 | 3′ spacer 17 | 2690 | 12 | ++++ | + | + | ++ |
| 5′ spacer 11 | 607 | 12 | 3′ spacer 5 | 1543 | 12 | + | + | + | + |
| 5′ spacer 11 | 607 | 12 | 3′ spacer 13 | 2315 | 10 | +++ | + | + | +++ |
| 5′ spacer 11 | 607 | 12 | 3′ spacer 13 | 2316 | 12 | ++ | + | + | ++ |
| 5′ spacer 11 | 607 | 12 | 3′ spacer 14 | 2333 | 10 | ++++ | + | NA | +++ |
| 5′ spacer 11 | 607 | 12 | 3′ spacer 14 | 2334 | 12 | ++++ | + | NA | ++++ |
| 5′ spacer 11 | 607 | 12 | 3′ spacer 6 | 1880 | 11 | ++ | + | + | + |
| 5′ spacer 11 | 607 | 12 | 3′ spacer 6 | 1881 | 13 | +++ | + | + | + |
| 5′ spacer 11 | 607 | 12 | 3′ spacer 15 | 2352 | 12 | +++ | + | + | + |
| 5′ spacer 11 | 607 | 12 | 3′ spacer 7 | 1898 | 11 | ++++ | + | + | + |
| 5′ spacer 11 | 607 | 12 | 3′ spacer 7 | 1899 | 13 | ++++ | + | + | + |
| 5′ spacer 11 | 607 | 12 | 3′ spacer 2 | 1399 | 12 | + | + | + | ++ |
| 5′ spacer 12 | 927 | 11 | 3′ spacer 8 | 1916 | 10 | +++ | + | + | + |
| 5′ spacer 12 | 927 | 11 | 3′ spacer 9 | 1934 | 10 | + | + | + | ++ |
| 5′ spacer 12 | 927 | 11 | 3′ spacer 9 | 1935 | 12 | + | NA | NA | + |
| 5′ spacer 12 | 927 | 11 | 3′ spacer 10 | 1952 | 10 | +++ | + | + | + |
| 5′ spacer 12 | 927 | 11 | 3′ spacer 10 | 1953 | 12 | ++++ | + | + | + |
| 5′ spacer 12 | 927 | 11 | 3′ spacer 11 | 2279 | 10 | +++ | + | + | + |
| 5′ spacer 12 | 927 | 11 | 3′ spacer 11 | 2280 | 12 | +++ | + | + | + |
| 5′ spacer 12 | 927 | 11 | 3′ spacer 16 | 2369 | 10 | +++ | + | + | + |
| 5′ spacer 12 | 927 | 11 | 3′ spacer 16 | 2370 | 12 | +++ | + | + | + |
| 5′ spacer 12 | 927 | 11 | 3′ spacer 12 | 2297 | 10 | ++ | + | + | + |
| 5′ spacer 12 | 927 | 11 | 3′ spacer 12 | 2298 | 12 | +++ | + | + | ++ |
| 5′ spacer 12 | 927 | 11 | 3′ spacer 17 | 2689 | 10 | ++++ | + | + | + |
| 5′ spacer 12 | 927 | 11 | 3′ spacer 17 | 2690 | 12 | ++++ | + | + | + |
| 5′ spacer 12 | 927 | 11 | 3′ spacer 5 | 1543 | 12 | ++++ | + | + | ++ |
| 5′ spacer 12 | 927 | 11 | 3′ spacer 13 | 2315 | 10 | ++++ | + | + | ++ |
| 5′ spacer 12 | 927 | 11 | 3′ spacer 13 | 2316 | 12 | ++++ | + | + | +++ |
| 5′ spacer 12 | 927 | 11 | 3′ spacer 14 | 2333 | 10 | ++++ | + | + | +++ |
| 5′ spacer 12 | 927 | 11 | 3′ spacer 14 | 2334 | 12 | ++++ | + | + | +++ |
| 5′ spacer 12 | 927 | 11 | 3′ spacer 6 | 1880 | 11 | + | + | + | + |
| 5′ spacer 12 | 927 | 11 | 3′ spacer 6 | 1881 | 13 | ++ | + | + | + |
| 5′ spacer 12 | 927 | 11 | 3′ spacer 15 | 2351 | 10 | +++ | + | + | + |
| 5′ spacer 12 | 927 | 11 | 3′ spacer 15 | 2352 | 12 | +++ | + | + | + |
| 5′ spacer 12 | 927 | 11 | 3′ spacer 7 | 1898 | 11 | + | + | + | + |
| 5′ spacer 12 | 927 | 11 | 3′ spacer 7 | 1899 | 13 | + | + | + | + |
| 5′ spacer 12 | 927 | 11 | 3′ spacer 2 | 1399 | 12 | +++ | + | + | + |
| 5′ spacer 12 | 928 | 13 | 3′ spacer 8 | 1916 | 10 | + | + | + | + |
| 5′ spacer 12 | 928 | 13 | 3′ spacer 9 | 1935 | 12 | + | + | + | ++ |
| 5′ spacer 12 | 928 | 13 | 3′ spacer 10 | 1952 | 10 | ++++ | + | + | + |
| 5′ spacer 12 | 928 | 13 | 3′ spacer 10 | 1953 | 12 | ++++ | + | + | + |
| 5′ spacer 12 | 928 | 13 | 3′ spacer 11 | 2279 | 10 | +++ | + | + | + |
| 5′ spacer 12 | 928 | 13 | 3′ spacer 11 | 2280 | 12 | +++ | + | + | + |
| 5′ spacer 12 | 928 | 13 | 3′ spacer 16 | 2369 | 10 | +++ | + | + | + |
| 5′ spacer 12 | 928 | 13 | 3′ spacer 16 | 2370 | 12 | ++ | + | + | + |
| 5′ spacer 12 | 928 | 13 | 3′ spacer 12 | 2297 | 10 | +++ | + | + | + |
| 5′ spacer 12 | 928 | 13 | 3′ spacer 12 | 2298 | 12 | +++ | + | + | + |
| 5′ spacer 12 | 928 | 13 | 3′ spacer 17 | 2689 | 10 | ++++ | + | + | + |
| 5′ spacer 12 | 928 | 13 | 3′ spacer 17 | 2690 | 12 | ++++ | + | + | + |
| 5′ spacer 12 | 928 | 13 | 3′ spacer 5 | 1543 | 12 | ++++ | + | + | ++ |
| 5′ spacer 12 | 928 | 13 | 3′ spacer 13 | 2315 | 10 | ++++ | + | + | ++ |
| 5′ spacer 12 | 928 | 13 | 3′ spacer 13 | 2316 | 12 | ++++ | ++ | + | ++++ |
| 5′ spacer 12 | 928 | 13 | 3′ spacer 14 | 2333 | 10 | +++ | + | + | ++ |
| 5′ spacer 12 | 928 | 13 | 3′ spacer 14 | 2334 | 12 | ++++ | + | + | +++ |
| 5′ spacer 12 | 928 | 13 | 3′ spacer 6 | 1880 | 11 | + | + | + | ++ |
| 5′ spacer 12 | 928 | 13 | 3′ spacer 6 | 1881 | 13 | ++ | + | + | + |
| 5′ spacer 12 | 928 | 13 | 3′ spacer 15 | 2351 | 10 | ++ | + | + | + |
| 5′ spacer 12 | 928 | 13 | 3′ spacer 15 | 2352 | 12 | +++ | + | + | + |
| 5′ spacer 12 | 928 | 13 | 3′ spacer 7 | 1898 | 11 | + | + | + | + |
| 5′ spacer 12 | 928 | 13 | 3′ spacer 7 | 1899 | 13 | + | + | + | + |
| 5′ spacer 12 | 928 | 13 | 3′ spacer 2 | 1399 | 12 | ++ | + | + | + |
| 5′ spacer 13 | 945 | 10 | 3′ spacer 8 | 1916 | 10 | ++++ | + | + | ++ |
| 5′ spacer 13 | 945 | 10 | 3′ spacer 9 | 1934 | 10 | ++++ | + | + | +++ |
| 5′ spacer 13 | 945 | 10 | 3′ spacer 9 | 1935 | 12 | ++++ | + | + | +++ |
| 5′ spacer 13 | 945 | 10 | 3′ spacer 10 | 1952 | 10 | ++++ | + | NA | ++ |
| 5′ spacer 13 | 945 | 10 | 3′ spacer 10 | 1953 | 12 | ++++ | + | NA | ++ |
| 5′ spacer 13 | 945 | 10 | 3′ spacer 11 | 2279 | 10 | ++++ | + | NA | ++ |
| 5′ spacer 13 | 945 | 10 | 3′ spacer 11 | 2280 | 12 | ++++ | + | NA | ++ |
| 5′ spacer 13 | 945 | 10 | 3′ spacer 16 | 2369 | 10 | ++++ | + | NA | + |
| 5′ spacer 13 | 945 | 10 | 3′ spacer 16 | 2370 | 12 | ++++ | + | NA | + |
| 5′ spacer 13 | 945 | 10 | 3′ spacer 12 | 2297 | 10 | +++ | + | NA | + |
| 5′ spacer 13 | 945 | 10 | 3′ spacer 12 | 2298 | 12 | ++++ | + | NA | + |
| 5′ spacer 13 | 945 | 10 | 3′ spacer 17 | 2689 | 10 | ++++ | + | NA | + |
| 5′ spacer 13 | 945 | 10 | 3′ spacer 17 | 2690 | 12 | ++++ | + | NA | + |
| 5′ spacer 13 | 945 | 10 | 3′ spacer 6 | 1880 | 11 | ++++ | + | + | + |
| 5′ spacer 13 | 945 | 10 | 3′ spacer 6 | 1881 | 13 | ++++ | + | + | + |
| 5′ spacer 13 | 945 | 10 | 3′ spacer 15 | 2351 | 10 | ++++ | + | + | ++++ |
| 5′ spacer 13 | 945 | 10 | 3′ spacer 15 | 2352 | 12 | ++++ | + | + | ++++ |
| 5′ spacer 13 | 945 | 10 | 3′ spacer 7 | 1898 | 11 | ++++ | + | + | ++ |
| 5′ spacer 13 | 945 | 10 | 3′ spacer 7 | 1899 | 13 | ++++ | + | + | ++ |
| 5′ spacer 13 | 945 | 10 | 3′ spacer 2 | 1399 | 12 | ++++ | + | + | ++ |
| 5′ spacer 13 | 946 | 12 | 3′ spacer 8 | 1916 | 10 | ++++ | + | + | ++ |
| 5′ spacer 13 | 946 | 12 | 3′ spacer 9 | 1934 | 10 | ++++ | + | + | +++ |
| 5′ spacer 13 | 946 | 12 | 3′ spacer 9 | 1935 | 12 | ++++ | + | + | +++ |
| 5′ spacer 13 | 946 | 12 | 3′ spacer 10 | 1952 | 10 | ++++ | + | NA | ++ |
| 5′ spacer 13 | 946 | 12 | 3′ spacer 10 | 1953 | 12 | ++++ | + | NA | ++ |
| 5′ spacer 13 | 946 | 12 | 3′ spacer 11 | 2279 | 10 | ++++ | + | NA | ++ |
| 5′ spacer 13 | 946 | 12 | 3′ spacer 11 | 2280 | 12 | ++++ | + | NA | ++ |
| 5′ spacer 13 | 946 | 12 | 3′ spacer 16 | 2369 | 10 | ++++ | + | NA | + |
| 5′ spacer 13 | 946 | 12 | 3′ spacer 16 | 2370 | 12 | ++++ | + | NA | + |
| 5′ spacer 13 | 946 | 12 | 3′ spacer 12 | 2297 | 10 | +++ | + | NA | + |
| 5′ spacer 13 | 946 | 12 | 3′ spacer 12 | 2298 | 12 | ++++ | + | NA | + |
| 5′ spacer 13 | 946 | 12 | 3′ spacer 17 | 2689 | 10 | +++ | + | NA | + |
| 5′ spacer 13 | 946 | 12 | 3′ spacer 17 | 2690 | 12 | ++++ | + | NA | + |
| 5′ spacer 13 | 946 | 12 | 3′ spacer 6 | 1880 | 11 | ++++ | + | + | + |
| 5′ spacer 13 | 946 | 12 | 3′ spacer 6 | 1881 | 13 | ++++ | + | + | + |
| 5′ spacer 13 | 946 | 12 | 3′ spacer 15 | 2351 | 10 | ++++ | + | + | ++++ |
| 5′ spacer 13 | 946 | 12 | 3′ spacer 15 | 2352 | 12 | ++++ | + | + | ++++ |
| 5′ spacer 13 | 946 | 12 | 3′ spacer 7 | 1898 | 11 | ++++ | + | + | ++ |
| 5′ spacer 13 | 946 | 12 | 3′ spacer 7 | 1899 | 13 | ++++ | + | + | ++ |
| 5′ spacer 13 | 946 | 12 | 3′ spacer 2 | 1399 | 12 | ++++ | + | + | ++ |
| 5′ spacer 14 | 963 | 10 | 3′ spacer 8 | 1916 | 10 | ++++ | + | + | +++ |
| 5′ spacer 14 | 963 | 10 | 3′ spacer 9 | 1934 | 10 | ++++ | + | + | +++ |
| 5′ spacer 14 | 963 | 10 | 3′ spacer 9 | 1935 | 12 | ++++ | + | + | +++ |
| 5′ spacer 14 | 963 | 10 | 3′ spacer 10 | 1952 | 10 | ++++ | + | + | ++ |
| 5′ spacer 14 | 963 | 10 | 3′ spacer 10 | 1953 | 12 | ++++ | + | + | ++ |
| 5′ spacer 14 | 963 | 10 | 3′ spacer 11 | 2279 | 10 | ++++ | + | + | ++ |
| 5′ spacer 14 | 963 | 10 | 3′ spacer 11 | 2280 | 12 | ++++ | + | + | ++ |
| 5′ spacer 14 | 963 | 10 | 3′ spacer 16 | 2369 | 10 | ++++ | + | + | +++ |
| 5′ spacer 14 | 963 | 10 | 3′ spacer 16 | 2370 | 12 | ++++ | + | + | +++ |
| 5′ spacer 14 | 963 | 10 | 3′ spacer 12 | 2297 | 10 | ++++ | + | NA | ++ |
| 5′ spacer 14 | 963 | 10 | 3′ spacer 12 | 2298 | 12 | ++++ | + | NA | ++ |
| 5′ spacer 14 | 963 | 10 | 3′ spacer 17 | 2689 | 10 | ++++ | + | NA | + |
| 5′ spacer 14 | 963 | 10 | 3′ spacer 17 | 2690 | 12 | ++++ | + | NA | + |
| 5′ spacer 14 | 963 | 10 | 3′ spacer 5 | 1543 | 12 | ++++ | + | NA | + |
| 5′ spacer 14 | 963 | 10 | 3′ spacer 6 | 1880 | 11 | ++++ | + | + | + |
| 5′ spacer 14 | 963 | 10 | 3′ spacer 6 | 1881 | 13 | ++++ | + | + | + |
| 5′ spacer 14 | 963 | 10 | 3′ spacer 15 | 2351 | 10 | ++++ | + | + | + |
| 5′ spacer 14 | 963 | 10 | 3′ spacer 15 | 2352 | 12 | ++++ | + | + | + |
| 5′ spacer 14 | 963 | 10 | 3′ spacer 7 | 1898 | 11 | ++++ | + | + | +++ |
| 5′ spacer 14 | 963 | 10 | 3′ spacer 7 | 1899 | 13 | ++++ | + | + | +++ |
| 5′ spacer 14 | 963 | 10 | 3′ spacer 2 | 1399 | 12 | ++++ | + | + | +++ |
| 5′ spacer 14 | 964 | 12 | 3′ spacer 8 | 1916 | 10 | ++++ | + | + | +++ |
| 5′ spacer 14 | 964 | 12 | 3′ spacer 9 | 1934 | 10 | ++++ | + | + | +++ |
| 5′ spacer 14 | 964 | 12 | 3′ spacer 9 | 1935 | 12 | ++++ | + | + | +++ |
| 5′ spacer 14 | 964 | 12 | 3′ spacer 10 | 1952 | 10 | ++++ | + | + | ++ |
| 5′ spacer 14 | 964 | 12 | 3′ spacer 10 | 1953 | 12 | ++++ | + | + | ++ |
| 5′ spacer 14 | 964 | 12 | 3′ spacer 11 | 2279 | 10 | ++++ | + | + | ++ |
| 5′ spacer 14 | 964 | 12 | 3′ spacer 11 | 2280 | 12 | ++++ | + | + | ++ |
| 5′ spacer 14 | 964 | 12 | 3′ spacer 16 | 2369 | 10 | ++++ | + | + | +++ |
| 5′ spacer 14 | 964 | 12 | 3′ spacer 16 | 2370 | 12 | ++++ | + | + | +++ |
| 5′ spacer 14 | 964 | 12 | 3′ spacer 12 | 2297 | 10 | ++++ | + | NA | ++ |
| 5′ spacer 14 | 964 | 12 | 3′ spacer 12 | 2298 | 12 | ++++ | + | NA | ++ |
| 5′ spacer 14 | 964 | 12 | 3′ spacer 17 | 2689 | 10 | ++++ | + | NA | + |
| 5′ spacer 14 | 964 | 12 | 3′ spacer 17 | 2690 | 12 | ++++ | + | NA | + |
| 5′ spacer 14 | 964 | 12 | 3′ spacer 5 | 1543 | 12 | ++++ | + | NA | + |
| 5′ spacer 14 | 964 | 12 | 3′ spacer 6 | 1880 | 11 | ++++ | + | + | + |
| 5′ spacer 14 | 964 | 12 | 3′ spacer 6 | 1881 | 13 | +++ | + | + | + |
| 5′ spacer 14 | 964 | 12 | 3′ spacer 15 | 2351 | 10 | +++ | + | + | + |
| 5′ spacer 14 | 964 | 12 | 3′ spacer 15 | 2352 | 12 | ++++ | + | + | + |
| 5′ spacer 14 | 964 | 12 | 3′ spacer 7 | 1898 | 11 | ++++ | + | + | +++ |
| 5′ spacer 14 | 964 | 12 | 3′ spacer 7 | 1899 | 13 | ++++ | + | + | +++ |
| 5′ spacer 14 | 964 | 12 | 3′ spacer 2 | 1399 | 12 | ++++ | + | + | +++ |
| 5′ spacer 15 | 981 | 10 | 3′ spacer 8 | 1916 | 10 | ++ | + | + | + |
| 5′ spacer 15 | 981 | 10 | 3′ spacer 9 | 1934 | 10 | ++++ | + | + | + |
| 5′ spacer 15 | 981 | 10 | 3′ spacer 9 | 1935 | 12 | + | + | + | + |
| 5′ spacer 15 | 981 | 10 | 3′ spacer 10 | 1952 | 10 | ++++ | + | + | + |
| 5′ spacer 15 | 981 | 10 | 3′ spacer 10 | 1953 | 12 | +++ | + | + | + |
| 5′ spacer 15 | 981 | 10 | 3′ spacer 11 | 2279 | 10 | ++ | + | + | + |
| 5′ spacer 15 | 981 | 10 | 3′ spacer 11 | 2280 | 12 | +++ | + | + | + |
| 5′ spacer 15 | 981 | 10 | 3′ spacer 16 | 2369 | 10 | +++ | + | + | + |
| 5′ spacer 15 | 981 | 10 | 3′ spacer 16 | 2370 | 12 | +++ | + | + | + |
| 5′ spacer 15 | 981 | 10 | 3′ spacer 12 | 2297 | 10 | +++ | + | + | ++ |
| 5′ spacer 15 | 981 | 10 | 3′ spacer 12 | 2298 | 12 | +++ | + | + | ++ |
| 5′ spacer 15 | 981 | 10 | 3′ spacer 17 | 2689 | 10 | +++ | + | + | +++ |
| 5′ spacer 15 | 981 | 10 | 3′ spacer 17 | 2690 | 12 | ++++ | + | + | +++ |
| 5′ spacer 15 | 981 | 10 | 3′ spacer 5 | 1543 | 12 | ++++ | + | + | ++ |
| 5′ spacer 15 | 981 | 10 | 3′ spacer 13 | 2315 | 10 | +++ | + | + | ++ |
| 5′ spacer 15 | 981 | 10 | 3′ spacer 13 | 2316 | 12 | ++++ | + | + | + |
| 5′ spacer 15 | 981 | 10 | 3′ spacer 14 | 2333 | 10 | ++++ | + | + | ++ |
| 5′ spacer 15 | 981 | 10 | 3′ spacer 14 | 2334 | 12 | ++++ | + | + | +++ |
| 5′ spacer 15 | 981 | 10 | 3′ spacer 6 | 1880 | 11 | ++ | + | + | + |
| 5′ spacer 15 | 981 | 10 | 3′ spacer 6 | 1881 | 13 | ++++ | + | + | + |
| 5′ spacer 15 | 981 | 10 | 3′ spacer 15 | 2351 | 10 | ++ | + | + | + |
| 5′ spacer 15 | 981 | 10 | 3′ spacer 15 | 2352 | 12 | +++ | + | + | + |
| 5′ spacer 15 | 981 | 10 | 3′ spacer 7 | 1898 | 11 | +++ | + | + | + |
| 5′ spacer 15 | 981 | 10 | 3′ spacer 7 | 1899 | 13 | +++ | + | + | + |
| 5′ spacer 15 | 981 | 10 | 3′ spacer 2 | 1399 | 12 | ++ | + | + | + |
| 5′ spacer 15 | 982 | 12 | 3′ spacer 8 | 1916 | 10 | ++++ | + | + | + |
| 5′ spacer 15 | 982 | 12 | 3′ spacer 9 | 1934 | 10 | +++ | + | + | + |
| 5′ spacer 15 | 982 | 12 | 3′ spacer 9 | 1935 | 12 | ++ | + | + | + |
| 5′ spacer 15 | 982 | 12 | 3′ spacer 10 | 1952 | 10 | ++ | + | + | + |
| 5′ spacer 15 | 982 | 12 | 3′ spacer 10 | 1953 | 12 | ++ | + | + | + |
| 5′ spacer 15 | 982 | 12 | 3′ spacer 11 | 2279 | 10 | + | + | + | + |
| 5′ spacer 15 | 982 | 12 | 3′ spacer 11 | 2280 | 12 | ++ | + | + | + |
| 5′ spacer 15 | 982 | 12 | 3′ spacer 16 | 2369 | 10 | + | + | + | ++ |
| 5′ spacer 15 | 982 | 12 | 3′ spacer 16 | 2370 | 12 | + | + | + | ++ |
| 5′ spacer 15 | 982 | 12 | 3′ spacer 12 | 2297 | 10 | + | + | + | ++ |
| 5′ spacer 15 | 982 | 12 | 3′ spacer 12 | 2298 | 12 | + | + | + | + |
| 5′ spacer 15 | 982 | 12 | 3′ spacer 17 | 2689 | 10 | + | + | + | ++++ |
| 5′ spacer 15 | 982 | 12 | 3′ spacer 17 | 2690 | 12 | + | + | + | ++++ |
| 5′ spacer 15 | 982 | 12 | 3′ spacer 5 | 1543 | 12 | ++ | + | + | ++++ |
| 5′ spacer 15 | 982 | 12 | 3′ spacer 13 | 2315 | 10 | +++ | + | + | ++ |
| 5′ spacer 15 | 982 | 12 | 3′ spacer 13 | 2316 | 12 | +++ | + | + | ++ |
| 5′ spacer 15 | 982 | 12 | 3′ spacer 14 | 2333 | 10 | + | + | + | +++ |
| 5′ spacer 15 | 982 | 12 | 3′ spacer 14 | 2334 | 12 | +++ | + | + | ++ |
| 5′ spacer 15 | 982 | 12 | 3′ spacer 6 | 1880 | 11 | + | + | + | ++ |
| 5′ spacer 15 | 982 | 12 | 3′ spacer 6 | 1881 | 13 | ++ | + | + | + |
| 5′ spacer 15 | 982 | 12 | 3′ spacer 15 | 2351 | 10 | + | + | + | + |
| 5′ spacer 15 | 982 | 12 | 3′ spacer 15 | 2352 | 12 | ++ | + | + | + |
| 5′ spacer 15 | 982 | 12 | 3′ spacer 7 | 1898 | 11 | ++ | + | + | + |
| 5′ spacer 15 | 982 | 12 | 3′ spacer 7 | 1899 | 13 | +++ | + | + | + |
| 5′ spacer 15 | 982 | 12 | 3′ spacer 2 | 1399 | 12 | + | + | + | + |
| 5′ spacer 16 | 999 | 10 | 3′ spacer 8 | 1916 | 10 | ++++ | + | + | + |
| 5′ spacer 16 | 999 | 10 | 3′ spacer 9 | 1934 | 10 | ++++ | + | + | + |
| 5′ spacer 16 | 999 | 10 | 3′ spacer 9 | 1935 | 12 | ++++ | + | + | + |
| 5′ spacer 16 | 999 | 10 | 3′ spacer 10 | 1952 | 10 | ++++ | + | + | + |
| 5′ spacer 16 | 999 | 10 | 3′ spacer 10 | 1953 | 12 | ++++ | + | + | + |
| 5′ spacer 16 | 999 | 10 | 3′ spacer 11 | 2279 | 10 | +++ | + | + | + |
| 5′ spacer 16 | 999 | 10 | 3′ spacer 11 | 2280 | 12 | ++++ | + | + | + |
| 5′ spacer 16 | 999 | 10 | 3′ spacer 16 | 2369 | 10 | ++++ | + | + | + |
| 5′ spacer 16 | 999 | 10 | 3′ spacer 16 | 2370 | 12 | ++++ | + | + | + |
| 5′ spacer 16 | 999 | 10 | 3′ spacer 12 | 2297 | 10 | +++ | + | + | + |
| 5′ spacer 16 | 999 | 10 | 3′ spacer 12 | 2298 | 12 | + | NA | NA | ++ |
| 5′ spacer 16 | 999 | 10 | 3′ spacer 17 | 2689 | 10 | ++++ | + | + | ++ |
| 5′ spacer 16 | 999 | 10 | 3′ spacer 17 | 2690 | 12 | ++++ | + | + | ++ |
| 5′ spacer 16 | 999 | 10 | 3′ spacer 5 | 1543 | 12 | ++++ | + | + | +++ |
| 5′ spacer 16 | 999 | 10 | 3′ spacer 13 | 2315 | 10 | ++++ | + | + | +++ |
| 5′ spacer 16 | 999 | 10 | 3′ spacer 13 | 2316 | 12 | ++++ | + | + | +++ |
| 5′ spacer 16 | 999 | 10 | 3′ spacer 14 | 2333 | 10 | ++++ | + | + | +++ |
| 5′ spacer 16 | 999 | 10 | 3′ spacer 14 | 2334 | 12 | ++++ | + | + | ++ |
| 5′ spacer 16 | 999 | 10 | 3′ spacer 6 | 1880 | 11 | +++ | + | + | + |
| 5′ spacer 16 | 999 | 10 | 3′ spacer 6 | 1881 | 13 | +++ | + | + | + |
| 5′ spacer 16 | 999 | 10 | 3′ spacer 15 | 2351 | 10 | +++ | + | + | + |
| 5′ spacer 16 | 999 | 10 | 3′ spacer 15 | 2352 | 12 | +++ | + | + | + |
| 5′ spacer 16 | 999 | 10 | 3′ spacer 7 | 1898 | 11 | +++ | + | + | + |
| 5′ spacer 16 | 999 | 10 | 3′ spacer 7 | 1899 | 13 | ++++ | + | + | + |
| 5′ spacer 16 | 999 | 10 | 3′ spacer 2 | 1399 | 12 | +++ | + | + | + |
| 5′ spacer 16 | 1000 | 12 | 3′ spacer 8 | 1916 | 10 | ++++ | + | + | + |
| 5′ spacer 16 | 1000 | 12 | 3′ spacer 9 | 1934 | 10 | ++++ | + | + | + |
| 5′ spacer 16 | 1000 | 12 | 3′ spacer 9 | 1935 | 12 | ++++ | + | + | + |
| 5′ spacer 16 | 1000 | 12 | 3′ spacer 10 | 1952 | 10 | ++++ | + | + | + |
| 5′ spacer 16 | 1000 | 12 | 3′ spacer 10 | 1953 | 12 | ++++ | + | + | + |
| 5′ spacer 16 | 1000 | 12 | 3′ spacer 11 | 2279 | 10 | +++ | + | + | + |
| 5′ spacer 16 | 1000 | 12 | 3′ spacer 11 | 2280 | 12 | ++++ | + | + | + |
| 5′ spacer 16 | 1000 | 12 | 3′ spacer 16 | 2369 | 10 | ++++ | + | + | + |
| 5′ spacer 16 | 1000 | 12 | 3′ spacer 16 | 2370 | 12 | ++++ | + | + | + |
| 5′ spacer 16 | 1000 | 12 | 3′ spacer 12 | 2297 | 10 | +++ | + | + | + |
| 5′ spacer 16 | 1000 | 12 | 3′ spacer 12 | 2298 | 12 | +++ | + | + | + |
| 5′ spacer 16 | 1000 | 12 | 3′ spacer 17 | 2689 | 10 | ++++ | + | + | ++ |
| 5′ spacer 16 | 1000 | 12 | 3′ spacer 17 | 2690 | 12 | ++++ | + | + | ++ |
| 5′ spacer 16 | 1000 | 12 | 3′ spacer 5 | 1543 | 12 | ++++ | + | + | +++ |
| 5′ spacer 16 | 1000 | 12 | 3′ spacer 13 | 2315 | 10 | ++++ | + | + | +++ |
| 5′ spacer 16 | 1000 | 12 | 3′ spacer 13 | 2316 | 12 | ++++ | + | + | +++ |
| 5′ spacer 16 | 1000 | 12 | 3′ spacer 14 | 2333 | 10 | ++++ | + | + | +++ |
| 5′ spacer 16 | 1000 | 12 | 3′ spacer 14 | 2334 | 12 | ++++ | + | + | +++ |
| 5′ spacer 16 | 1000 | 12 | 3′ spacer 6 | 1880 | 11 | ++++ | + | + | + |
| 5′ spacer 16 | 1000 | 12 | 3′ spacer 6 | 1881 | 13 | ++++ | + | + | + |
| 5′ spacer 16 | 1000 | 12 | 3′ spacer 15 | 2351 | 10 | +++ | + | + | + |
| 5′ spacer 16 | 1000 | 12 | 3′ spacer 15 | 2352 | 12 | +++ | + | + | + |
| 5′ spacer 16 | 1000 | 12 | 3′ spacer 7 | 1898 | 11 | ++++ | + | + | + |
| 5′ spacer 16 | 1000 | 12 | 3′ spacer 7 | 1899 | 13 | +++ | + | + | + |
| 5′ spacer 16 | 1000 | 12 | 3′ spacer 2 | 1399 | 12 | +++ | + | + | + |
| 5′ spacer 17 | 1017 | 10 | 3′ spacer 8 | 1916 | 10 | ++++ | + | + | + |
| 5′ spacer 17 | 1017 | 10 | 3′ spacer 9 | 1934 | 10 | +++ | + | + | + |
| 5′ spacer 17 | 1017 | 10 | 3′ spacer 9 | 1935 | 12 | ++ | + | + | + |
| 5′ spacer 17 | 1017 | 10 | 3′ spacer 10 | 1952 | 10 | ++++ | + | + | + |
| 5′ spacer 17 | 1017 | 10 | 3′ spacer 10 | 1953 | 12 | ++++ | + | + | + |
| 5′ spacer 17 | 1017 | 10 | 3′ spacer 11 | 2279 | 10 | +++ | + | + | + |
| 5′ spacer 17 | 1017 | 10 | 3′ spacer 11 | 2280 | 12 | +++ | + | + | + |
| 5′ spacer 17 | 1017 | 10 | 3′ spacer 16 | 2369 | 10 | ++++ | + | + | + |
| 5′ spacer 17 | 1017 | 10 | 3′ spacer 16 | 2370 | 12 | +++ | + | + | + |
| 5′ spacer 17 | 1017 | 10 | 3′ spacer 12 | 2297 | 10 | +++ | + | + | + |
| 5′ spacer 17 | 1017 | 10 | 3′ spacer 12 | 2298 | 12 | +++ | + | + | ++ |
| 5′ spacer 17 | 1017 | 10 | 3′ spacer 17 | 2689 | 10 | ++++ | + | + | + |
| 5′ spacer 17 | 1017 | 10 | 3′ spacer 17 | 2690 | 12 | ++++ | + | + | + |
| 5′ spacer 17 | 1017 | 10 | 3′ spacer 5 | 1543 | 12 | ++++ | + | + | +++ |
| 5′ spacer 17 | 1017 | 10 | 3′ spacer 13 | 2315 | 10 | ++++ | + | + | +++ |
| 5′ spacer 17 | 1017 | 10 | 3′ spacer 13 | 2316 | 12 | ++++ | + | + | ++++ |
| 5′ spacer 17 | 1017 | 10 | 3′ spacer 14 | 2333 | 10 | ++++ | + | + | ++++ |
| 5′ spacer 17 | 1017 | 10 | 3′ spacer 14 | 2334 | 12 | ++++ | + | + | ++++ |
| 5′ spacer 17 | 1017 | 10 | 3′ spacer 6 | 1880 | 11 | +++ | + | + | + |
| 5′ spacer 17 | 1017 | 10 | 3′ spacer 6 | 1881 | 13 | ++ | + | + | + |
| 5′ spacer 17 | 1017 | 10 | 3′ spacer 15 | 2351 | 10 | +++ | + | + | + |
| 5′ spacer 17 | 1017 | 10 | 3′ spacer 15 | 2352 | 12 | +++ | + | + | + |
| 5′ spacer 17 | 1017 | 10 | 3′ spacer 7 | 1898 | 11 | ++ | + | + | + |
| 5′ spacer 17 | 1017 | 10 | 3′ spacer 7 | 1899 | 13 | +++ | + | + | + |
| 5′ spacer 17 | 1017 | 10 | 3′ spacer 2 | 1399 | 12 | +++ | + | + | + |
| 5′ spacer 17 | 1018 | 12 | 3′ spacer 8 | 1916 | 10 | +++ | + | + | + |
| 5′ spacer 17 | 1018 | 12 | 3′ spacer 9 | 1934 | 10 | +++ | + | + | + |
| 5′ spacer 17 | 1018 | 12 | 3′ spacer 9 | 1935 | 12 | +++ | + | + | + |
| 5′ spacer 17 | 1018 | 12 | 3′ spacer 10 | 1952 | 10 | ++++ | + | + | + |
| 5′ spacer 17 | 1018 | 12 | 3′ spacer 10 | 1953 | 12 | ++++ | + | + | + |
| 5′ spacer 17 | 1018 | 12 | 3′ spacer 11 | 2279 | 10 | +++ | + | + | + |
| 5′ spacer 17 | 1018 | 12 | 3′ spacer 11 | 2280 | 12 | +++ | + | + | + |
| 5′ spacer 17 | 1018 | 12 | 3′ spacer 16 | 2369 | 10 | +++ | + | + | + |
| 5′ spacer 17 | 1018 | 12 | 3′ spacer 16 | 2370 | 12 | +++ | + | + | + |
| 5′ spacer 17 | 1018 | 12 | 3′ spacer 12 | 2297 | 10 | ++ | + | + | + |
| 5′ spacer 17 | 1018 | 12 | 3′ spacer 12 | 2298 | 12 | ++++ | + | + | ++ |
| 5′ spacer 17 | 1018 | 12 | 3′ spacer 17 | 2689 | 10 | ++++ | + | + | + |
| 5′ spacer 17 | 1018 | 12 | 3′ spacer 17 | 2690 | 12 | ++++ | + | + | + |
| 5′ spacer 17 | 1018 | 12 | 3′ spacer 5 | 1543 | 12 | ++++ | + | + | +++ |
| 5′ spacer 17 | 1018 | 12 | 3′ spacer 13 | 2315 | 10 | ++++ | + | + | +++ |
| 5′ spacer 17 | 1018 | 12 | 3′ spacer 13 | 2316 | 12 | ++++ | + | + | ++++ |
| 5′ spacer 17 | 1018 | 12 | 3′ spacer 14 | 2333 | 10 | ++ | + | + | ++++ |
| 5′ spacer 17 | 1018 | 12 | 3′ spacer 14 | 2334 | 12 | ++ | + | + | ++++ |
| 5′ spacer 17 | 1018 | 12 | 3′ spacer 6 | 1880 | 11 | +++ | + | + | + |
| 5′ spacer 17 | 1018 | 12 | 3′ spacer 6 | 1881 | 13 | +++ | + | + | + |
| 5′ spacer 17 | 1018 | 12 | 3′ spacer 15 | 2351 | 10 | ++ | + | + | + |
| 5′ spacer 17 | 1018 | 12 | 3′ spacer 15 | 2352 | 12 | +++ | + | + | + |
| 5′ spacer 17 | 1018 | 12 | 3′ spacer 7 | 1898 | 11 | +++ | + | + | + |
| 5′ spacer 17 | 1018 | 12 | 3′ spacer 7 | 1899 | 13 | ++ | + | + | + |
| 5′ spacer 17 | 1018 | 12 | 3′ spacer 2 | 1399 | 12 | +++ | + | + | + |
| 5′ spacer 18 | 1035 | 10 | 3′ spacer 8 | 1916 | 10 | ++++ | + | + | + |
| 5′ spacer 18 | 1035 | 10 | 3′ spacer 9 | 1934 | 10 | ++ | + | + | + |
| 5′ spacer 18 | 1035 | 10 | 3′ spacer 9 | 1935 | 12 | + | + | + | + |
| 5′ spacer 18 | 1035 | 10 | 3′ spacer 10 | 1952 | 10 | ++++ | + | + | + |
| 5′ spacer 18 | 1035 | 10 | 3′ spacer 10 | 1953 | 12 | ++++ | + | + | + |
| 5′ spacer 18 | 1035 | 10 | 3′ spacer 11 | 2279 | 10 | +++ | + | + | + |
| 5′ spacer 18 | 1035 | 10 | 3′ spacer 11 | 2280 | 12 | +++ | + | + | + |
| 5′ spacer 18 | 1035 | 10 | 3′ spacer 16 | 2369 | 10 | +++ | + | + | + |
| 5′ spacer 18 | 1035 | 10 | 3′ spacer 16 | 2370 | 12 | +++ | + | + | + |
| 5′ spacer 18 | 1035 | 10 | 3′ spacer 12 | 2297 | 10 | ++++ | + | + | + |
| 5′ spacer 18 | 1035 | 10 | 3′ spacer 12 | 2298 | 12 | +++ | + | + | + |
| 5′ spacer 18 | 1035 | 10 | 3′ spacer 17 | 2689 | 10 | ++++ | + | + | ++ |
| 5′ spacer 18 | 1035 | 10 | 3′ spacer 17 | 2690 | 12 | ++++ | + | + | ++ |
| 5′ spacer 18 | 1035 | 10 | 3′ spacer 5 | 1543 | 12 | ++++ | + | + | +++ |
| 5′ spacer 18 | 1035 | 10 | 3′ spacer 13 | 2315 | 10 | ++++ | + | + | ++++ |
| 5′ spacer 18 | 1035 | 10 | 3′ spacer 13 | 2316 | 12 | ++++ | + | + | ++++ |
| 5′ spacer 18 | 1035 | 10 | 3′ spacer 14 | 2333 | 10 | ++++ | + | + | ++++ |
| 5′ spacer 18 | 1035 | 10 | 3′ spacer 14 | 2334 | 12 | ++++ | + | + | ++++ |
| 5′ spacer 18 | 1035 | 10 | 3′ spacer 6 | 1880 | 11 | ++ | + | + | + |
| 5′ spacer 18 | 1035 | 10 | 3′ spacer 6 | 1881 | 13 | + | + | + | + |
| 5′ spacer 18 | 1035 | 10 | 3′ spacer 15 | 2351 | 10 | +++ | + | + | + |
| 5′ spacer 18 | 1035 | 10 | 3′ spacer 15 | 2352 | 12 | +++ | + | + | + |
| 5′ spacer 18 | 1035 | 10 | 3′ spacer 7 | 1898 | 11 | + | + | + | + |
| 5′ spacer 18 | 1035 | 10 | 3′ spacer 7 | 1899 | 13 | ++ | + | + | + |
| 5′ spacer 18 | 1035 | 10 | 3′ spacer 2 | 1399 | 12 | +++ | + | + | + |
| 5′ spacer 18 | 1036 | 12 | 3′ spacer 8 | 1916 | 10 | ++++ | + | + | + |
| 5′ spacer 18 | 1036 | 12 | 3′ spacer 9 | 1934 | 10 | + | + | + | + |
| 5′ spacer 18 | 1036 | 12 | 3′ spacer 9 | 1935 | 12 | + | + | + | + |
| 5′ spacer 18 | 1036 | 12 | 3′ spacer 10 | 1952 | 10 | ++++ | + | + | + |
| 5′ spacer 18 | 1036 | 12 | 3′ spacer 10 | 1953 | 12 | ++++ | + | + | + |
| 5′ spacer 18 | 1036 | 12 | 3′ spacer 11 | 2279 | 10 | ++++ | + | + | + |
| 5′ spacer 18 | 1036 | 12 | 3′ spacer 11 | 2280 | 12 | +++ | + | + | + |
| 5′ spacer 18 | 1036 | 12 | 3′ spacer 16 | 2369 | 10 | +++ | + | + | + |
| 5′ spacer 18 | 1036 | 12 | 3′ spacer 16 | 2370 | 12 | +++ | + | + | + |
| 5′ spacer 18 | 1036 | 12 | 3′ spacer 12 | 2297 | 10 | +++ | + | + | + |
| 5′ spacer 18 | 1036 | 12 | 3′ spacer 12 | 2298 | 12 | +++ | + | + | ++ |
| 5′ spacer 18 | 1036 | 12 | 3′ spacer 17 | 2689 | 10 | ++++ | + | + | ++ |
| 5′ spacer 18 | 1036 | 12 | 3′ spacer 17 | 2690 | 12 | ++++ | + | + | ++ |
| 5′ spacer 18 | 1036 | 12 | 3′ spacer 5 | 1543 | 12 | ++++ | + | + | +++ |
| 5′ spacer 18 | 1036 | 12 | 3′ spacer 13 | 2315 | 10 | ++++ | + | + | ++++ |
| 5′ spacer 18 | 1036 | 12 | 3′ spacer 13 | 2316 | 12 | ++++ | + | + | ++++ |
| 5′ spacer 18 | 1036 | 12 | 3′ spacer 14 | 2333 | 10 | ++++ | + | + | ++++ |
| 5′ spacer 18 | 1036 | 12 | 3′ spacer 14 | 2334 | 12 | ++++ | + | + | ++++ |
| 5′ spacer 18 | 1036 | 12 | 3′ spacer 6 | 1880 | 11 | ++ | + | + | + |
| 5′ spacer 18 | 1036 | 12 | 3′ spacer 6 | 1881 | 13 | + | + | + | + |
| 5′ spacer 18 | 1036 | 12 | 3′ spacer 15 | 2351 | 10 | +++ | + | + | + |
| 5′ spacer 18 | 1036 | 12 | 3′ spacer 15 | 2352 | 12 | +++ | + | + | + |
| 5′ spacer 18 | 1036 | 12 | 3′ spacer 7 | 1898 | 11 | + | + | + | + |
| 5′ spacer 18 | 1036 | 12 | 3′ spacer 7 | 1899 | 13 | + | + | + | + |
| 5′ spacer 18 | 1036 | 12 | 3′ spacer 2 | 1399 | 12 | +++ | + | + | + |
| 5′ spacer 19 | 1357 | 10 | 3′ spacer 8 | 1916 | 10 | +++ | + | + | + |
| 5′ spacer 19 | 1357 | 10 | 3′ spacer 9 | 1934 | 10 | + | + | + | + |
| 5′ spacer 19 | 1357 | 10 | 3′ spacer 9 | 1935 | 12 | + | + | + | + |
| 5′ spacer 19 | 1357 | 10 | 3′ spacer 10 | 1952 | 10 | ++++ | + | + | + |
| 5′ spacer 19 | 1357 | 10 | 3′ spacer 10 | 1953 | 12 | ++++ | + | + | + |
| 5′ spacer 19 | 1357 | 10 | 3′ spacer 11 | 2279 | 10 | +++ | + | + | + |
| 5′ spacer 19 | 1357 | 10 | 3′ spacer 11 | 2280 | 12 | +++ | + | + | + |
| 5′ spacer 19 | 1357 | 10 | 3′ spacer 16 | 2369 | 10 | +++ | + | + | + |
| 5′ spacer 19 | 1357 | 10 | 3′ spacer 16 | 2370 | 12 | +++ | + | + | + |
| 5′ spacer 19 | 1357 | 10 | 3′ spacer 12 | 2297 | 10 | +++ | + | + | + |
| 5′ spacer 19 | 1357 | 10 | 3′ spacer 12 | 2298 | 12 | ++ | + | + | + |
| 5′ spacer 19 | 1357 | 10 | 3′ spacer 17 | 2689 | 10 | ++++ | + | + | + |
| 5′ spacer 19 | 1357 | 10 | 3′ spacer 17 | 2690 | 12 | ++++ | + | + | + |
| 5′ spacer 19 | 1357 | 10 | 3′ spacer 5 | 1543 | 12 | ++++ | + | + | + |
| 5′ spacer 19 | 1357 | 10 | 3′ spacer 13 | 2315 | 10 | +++ | + | + | ++ |
| 5′ spacer 19 | 1357 | 10 | 3′ spacer 13 | 2316 | 12 | +++ | + | + | ++ |
| 5′ spacer 19 | 1357 | 10 | 3′ spacer 14 | 2333 | 10 | +++ | + | + | +++ |
| 5′ spacer 19 | 1357 | 10 | 3′ spacer 14 | 2334 | 12 | +++ | + | + | ++ |
| 5′ spacer 19 | 1357 | 10 | 3′ spacer 6 | 1880 | 11 | + | + | + | + |
| 5′ spacer 19 | 1357 | 10 | 3′ spacer 6 | 1881 | 13 | + | + | + | + |
| 5′ spacer 19 | 1357 | 10 | 3′ spacer 15 | 2351 | 10 | +++ | + | + | + |
| 5′ spacer 19 | 1357 | 10 | 3′ spacer 15 | 2352 | 12 | +++ | + | + | + |
| 5′ spacer 19 | 1357 | 10 | 3′ spacer 7 | 1898 | 11 | + | + | + | + |
| 5′ spacer 19 | 1357 | 10 | 3′ spacer 7 | 1899 | 13 | + | + | + | + |
| 5′ spacer 19 | 1357 | 10 | 3′ spacer 2 | 1399 | 12 | ++ | + | + | + |
| 5′ spacer 19 | 1358 | 13 | 3′ spacer 8 | 1916 | 10 | +++ | + | + | + |
| 5′ spacer 19 | 1358 | 13 | 3′ spacer 9 | 1934 | 10 | + | + | + | + |
| 5′ spacer 19 | 1358 | 13 | 3′ spacer 9 | 1935 | 12 | + | + | + | + |
| 5′ spacer 19 | 1358 | 13 | 3′ spacer 10 | 1952 | 10 | +++ | + | + | + |
| 5′ spacer 19 | 1358 | 13 | 3′ spacer 10 | 1953 | 12 | ++++ | + | + | + |
| 5′ spacer 19 | 1358 | 13 | 3′ spacer 11 | 2279 | 10 | +++ | + | + | + |
| 5′ spacer 19 | 1358 | 13 | 3′ spacer 11 | 2280 | 12 | +++ | + | + | + |
| 5′ spacer 19 | 1358 | 13 | 3′ spacer 16 | 2369 | 10 | +++ | + | + | + |
| 5′ spacer 19 | 1358 | 13 | 3′ spacer 16 | 2370 | 12 | +++ | + | + | + |
| 5′ spacer 19 | 1358 | 13 | 3′ spacer 12 | 2297 | 10 | +++ | + | + | + |
| 5′ spacer 19 | 1358 | 13 | 3′ spacer 12 | 2298 | 12 | ++ | + | + | + |
| 5′ spacer 19 | 1358 | 13 | 3′ spacer 17 | 2689 | 10 | ++++ | + | + | + |
| 5′ spacer 19 | 1358 | 13 | 3′ spacer 17 | 2690 | 12 | ++++ | + | + | + |
| 5′ spacer 19 | 1358 | 13 | 3′ spacer 5 | 1543 | 12 | ++++ | + | + | + |
| 5′ spacer 19 | 1358 | 13 | 3′ spacer 13 | 2315 | 10 | +++ | + | + | +++ |
| 5′ spacer 19 | 1358 | 13 | 3′ spacer 13 | 2316 | 12 | +++ | + | + | ++ |
| 5′ spacer 19 | 1358 | 13 | 3′ spacer 14 | 2333 | 10 | ++++ | + | + | +++ |
| 5′ spacer 19 | 1358 | 13 | 3′ spacer 14 | 2334 | 12 | +++ | + | + | +++ |
| 5′ spacer 19 | 1358 | 13 | 3′ spacer 6 | 1880 | 11 | + | + | + | + |
| 5′ spacer 19 | 1358 | 13 | 3′ spacer 6 | 1881 | 13 | + | + | + | + |
| 5′ spacer 19 | 1358 | 13 | 3′ spacer 15 | 2351 | 10 | +++ | + | + | + |
| 5′ spacer 19 | 1358 | 13 | 3′ spacer 15 | 2352 | 12 | +++ | + | + | + |
| 5′ spacer 19 | 1358 | 13 | 3′ spacer 7 | 1898 | 11 | + | + | + | + |
| 5′ spacer 19 | 1358 | 13 | 3′ spacer 7 | 1899 | 13 | + | + | + | + |
| 5′ spacer 19 | 1358 | 13 | 3′ spacer 2 | 1399 | 12 | ++ | + | + | + |
| 5′ spacer 3 | 56 | 10 | 3′ spacer 8 | 1916 | 10 | ++ | + | + | + |
| 5′ spacer 3 | 56 | 10 | 3′ spacer 9 | 1934 | 10 | + | + | + | + |
| 5′ spacer 3 | 56 | 10 | 3′ spacer 9 | 1935 | 12 | +++ | + | + | + |
| 5′ spacer 3 | 56 | 10 | 3′ spacer 11 | 2279 | 10 | + | + | + | + |
| 5′ spacer 3 | 56 | 10 | 3′ spacer 11 | 2280 | 12 | + | + | + | + |
| 5′ spacer 3 | 56 | 10 | 3′ spacer 16 | 2369 | 10 | + | + | + | + |
| 5′ spacer 3 | 56 | 10 | 3′ spacer 12 | 2297 | 10 | ++ | NA | + | + |
| 5′ spacer 3 | 56 | 10 | 3′ spacer 17 | 2689 | 10 | + | NA | NA | + |
| 5′ spacer 3 | 56 | 10 | 3′ spacer 17 | 2690 | 12 | ++ | + | + | ++ |
| 5′ spacer 3 | 56 | 10 | 3′ spacer 5 | 1543 | 12 | ++ | + | + | ++ |
| 5′ spacer 3 | 56 | 10 | 3′ spacer 13 | 2315 | 10 | + | + | + | ++ |
| 5′ spacer 3 | 56 | 10 | 3′ spacer 13 | 2316 | 12 | + | + | + | ++ |
| 5′ spacer 3 | 56 | 10 | 3′ spacer 14 | 2333 | 10 | ++++ | + | + | +++ |
| 5′ spacer 3 | 56 | 10 | 3′ spacer 14 | 2334 | 12 | ++ | + | + | ++ |
| 5′ spacer 3 | 56 | 10 | 3′ spacer 6 | 1880 | 11 | + | + | + | + |
| 5′ spacer 3 | 56 | 10 | 3′ spacer 6 | 1881 | 13 | + | + | + | + |
| 5′ spacer 3 | 56 | 10 | 3′ spacer 15 | 2352 | 12 | + | + | + | + |
| 5′ spacer 3 | 56 | 10 | 3′ spacer 7 | 1898 | 11 | + | + | + | + |
| 5′ spacer 3 | 56 | 10 | 3′ spacer 7 | 1899 | 13 | + | + | + | + |
| 5′ spacer 3 | 56 | 10 | 3′ spacer 2 | 1399 | 12 | + | + | + | + |
| 5′ spacer 3 | 57 | 12 | 3′ spacer 8 | 1916 | 10 | + | + | + | + |
| 5′ spacer 3 | 57 | 12 | 3′ spacer 9 | 1934 | 10 | + | + | + | + |
| 5′ spacer 3 | 57 | 12 | 3′ spacer 9 | 1935 | 12 | ++ | + | + | + |
| 5′ spacer 3 | 57 | 12 | 3′ spacer 11 | 2279 | 10 | + | + | + | + |
| 5′ spacer 3 | 57 | 12 | 3′ spacer 16 | 2369 | 10 | ++ | + | + | + |
| 5′ spacer 3 | 57 | 12 | 3′ spacer 12 | 2297 | 10 | + | NA | NA | + |
| 5′ spacer 3 | 57 | 12 | 3′ spacer 17 | 2689 | 10 | + | + | NA | + |
| 5′ spacer 3 | 57 | 12 | 3′ spacer 17 | 2690 | 12 | + | NA | + | + |
| 5′ spacer 3 | 57 | 12 | 3′ spacer 5 | 1543 | 12 | ++ | + | + | +++ |
| 5′ spacer 3 | 57 | 12 | 3′ spacer 13 | 2315 | 10 | + | + | NA | ++ |
| 5′ spacer 3 | 57 | 12 | 3′ spacer 13 | 2316 | 12 | + | + | + | ++ |
| 5′ spacer 3 | 57 | 12 | 3′ spacer 14 | 2333 | 10 | ++++ | + | + | +++ |
| 5′ spacer 3 | 57 | 12 | 3′ spacer 14 | 2334 | 12 | +++ | + | + | ++ |
| 5′ spacer 3 | 57 | 12 | 3′ spacer 6 | 1880 | 11 | + | + | + | + |
| 5′ spacer 3 | 57 | 12 | 3′ spacer 6 | 1881 | 13 | + | + | + | + |
| 5′ spacer 3 | 57 | 12 | 3′ spacer 15 | 2352 | 12 | + | + | + | + |
| 5′ spacer 3 | 57 | 12 | 3′ spacer 7 | 1898 | 11 | + | + | + | + |
| 5′ spacer 3 | 57 | 12 | 3′ spacer 7 | 1899 | 13 | + | + | + | + |
| 5′ spacer 3 | 57 | 12 | 3′ spacer 2 | 1399 | 12 | + | + | + | + |
| 5′ spacer 7 | 534 | 10 | 3′ spacer 8 | 1916 | 10 | ++++ | + | + | ++ |
| 5′ spacer 7 | 534 | 10 | 3′ spacer 9 | 1934 | 10 | ++++ | + | + | ++ |
| 5′ spacer 7 | 534 | 10 | 3′ spacer 10 | 1952 | 10 | ++++ | + | + | ++ |
| 5′ spacer 7 | 534 | 10 | 3′ spacer 10 | 1953 | 12 | ++++ | + | + | ++ |
| 5′ spacer 7 | 534 | 10 | 3′ spacer 11 | 2279 | 10 | ++++ | + | + | ++ |
| 5′ spacer 7 | 534 | 10 | 3′ spacer 11 | 2280 | 12 | ++++ | + | + | ++ |
| 5′ spacer 7 | 534 | 10 | 3′ spacer 12 | 2297 | 10 | ++++ | + | NA | + |
| 5′ spacer 7 | 534 | 10 | 3′ spacer 12 | 2298 | 12 | ++++ | + | NA | + |
| 5′ spacer 7 | 534 | 10 | 3′ spacer 17 | 2689 | 10 | ++++ | + | NA | ++ |
| 5′ spacer 7 | 534 | 10 | 3′ spacer 17 | 2690 | 12 | ++++ | + | NA | ++ |
| 5′ spacer 7 | 534 | 10 | 3′ spacer 5 | 1543 | 12 | ++++ | + | NA | + |
| 5′ spacer 7 | 534 | 10 | 3′ spacer 6 | 1880 | 11 | ++++ | + | + | + |
| 5′ spacer 7 | 534 | 10 | 3′ spacer 6 | 1881 | 13 | ++++ | + | + | + |
| 5′ spacer 7 | 534 | 10 | 3′ spacer 15 | 2352 | 12 | ++++ | + | + | + |
| 5′ spacer 7 | 534 | 10 | 3′ spacer 7 | 1898 | 11 | ++++ | + | + | +++ |
| 5′ spacer 7 | 534 | 10 | 3′ spacer 7 | 1899 | 13 | ++++ | + | + | +++ |
| 5′ spacer 7 | 534 | 10 | 3′ spacer 2 | 1399 | 12 | ++++ | + | + | +++ |
| 5′ spacer 7 | 535 | 12 | 3′ spacer 8 | 1916 | 10 | ++++ | + | + | ++ |
| 5′ spacer 7 | 535 | 12 | 3′ spacer 9 | 1934 | 10 | ++++ | + | + | ++ |
| 5′ spacer 7 | 535 | 12 | 3′ spacer 9 | 1935 | 12 | ++++ | + | + | ++ |
| 5′ spacer 7 | 535 | 12 | 3′ spacer 10 | 1952 | 10 | ++++ | + | + | ++ |
| 5′ spacer 7 | 535 | 12 | 3′ spacer 10 | 1953 | 12 | ++++ | + | + | ++ |
| 5′ spacer 7 | 535 | 12 | 3′ spacer 11 | 2279 | 10 | +++ | + | + | ++ |
| 5′ spacer 7 | 535 | 12 | 3′ spacer 11 | 2280 | 12 | ++++ | + | + | ++ |
| 5′ spacer 7 | 535 | 12 | 3′ spacer 16 | 2369 | 10 | ++++ | + | NA | +++ |
| 5′ spacer 7 | 535 | 12 | 3′ spacer 12 | 2297 | 10 | ++++ | + | NA | + |
| 5′ spacer 7 | 535 | 12 | 3′ spacer 12 | 2298 | 12 | ++++ | + | NA | + |
| 5′ spacer 7 | 535 | 12 | 3′ spacer 17 | 2689 | 10 | ++++ | + | NA | ++ |
| 5′ spacer 7 | 535 | 12 | 3′ spacer 17 | 2690 | 12 | ++++ | + | NA | ++ |
| 5′ spacer 7 | 535 | 12 | 3′ spacer 5 | 1543 | 12 | ++++ | + | NA | + |
| 5′ spacer 7 | 535 | 12 | 3′ spacer 6 | 1880 | 11 | ++++ | + | + | + |
| 5′ spacer 7 | 535 | 12 | 3′ spacer 6 | 1881 | 13 | ++++ | + | + | + |
| 5′ spacer 7 | 535 | 12 | 3′ spacer 15 | 2352 | 12 | ++++ | + | + | + |
| 5′ spacer 7 | 535 | 12 | 3′ spacer 7 | 1898 | 11 | ++++ | + | + | +++ |
| 5′ spacer 7 | 535 | 12 | 3′ spacer 7 | 1899 | 13 | ++++ | + | + | +++ |
| 5′ spacer 7 | 535 | 12 | 3′ spacer 2 | 1399 | 12 | ++++ | + | + | +++ |
| 5′ spacer 8 | 552 | 10 | 3′ spacer 8 | 1916 | 10 | ++++ | + | + | ++ |
| 5′ spacer 8 | 552 | 10 | 3′ spacer 9 | 1934 | 10 | ++++ | + | + | ++ |
| 5′ spacer 8 | 552 | 10 | 3′ spacer 9 | 1935 | 12 | ++++ | + | + | ++ |
| 5′ spacer 8 | 552 | 10 | 3′ spacer 10 | 1952 | 10 | ++++ | + | + | +++ |
| 5′ spacer 8 | 552 | 10 | 3′ spacer 10 | 1953 | 12 | ++++ | + | + | +++ |
| 5′ spacer 8 | 552 | 10 | 3′ spacer 11 | 2279 | 10 | ++++ | + | + | ++ |
| 5′ spacer 8 | 552 | 10 | 3′ spacer 11 | 2280 | 12 | ++++ | + | + | ++ |
| 5′ spacer 8 | 552 | 10 | 3′ spacer 16 | 2369 | 10 | ++++ | + | + | ++ |
| 5′ spacer 8 | 552 | 10 | 3′ spacer 12 | 2297 | 10 | ++++ | + | NA | ++ |
| 5′ spacer 8 | 552 | 10 | 3′ spacer 12 | 2298 | 12 | ++++ | + | NA | ++ |
| 5′ spacer 8 | 552 | 10 | 3′ spacer 17 | 2689 | 10 | ++++ | + | NA | ++ |
| 5′ spacer 8 | 552 | 10 | 3′ spacer 17 | 2690 | 12 | ++++ | + | NA | ++ |
| 5′ spacer 8 | 552 | 10 | 3′ spacer 5 | 1543 | 12 | ++++ | + | NA | ++ |
| 5′ spacer 8 | 552 | 10 | 3′ spacer 13 | 2315 | 10 | +++ | + | NA | + |
| 5′ spacer 8 | 552 | 10 | 3′ spacer 13 | 2316 | 12 | ++++ | + | NA | + |
| 5′ spacer 8 | 552 | 10 | 3′ spacer 14 | 2333 | 10 | ++++ | + | NA | + |
| 5′ spacer 8 | 552 | 10 | 3′ spacer 14 | 2334 | 12 | ++++ | + | NA | + |
| 5′ spacer 8 | 552 | 10 | 3′ spacer 6 | 1880 | 11 | ++++ | + | + | + |
| 5′ spacer 8 | 552 | 10 | 3′ spacer 6 | 1881 | 13 | ++++ | + | + | + |
| 5′ spacer 8 | 552 | 10 | 3′ spacer 15 | 2352 | 12 | ++++ | + | + | + |
| 5′ spacer 8 | 552 | 10 | 3′ spacer 7 | 1898 | 11 | ++++ | + | + | ++ |
| 5′ spacer 8 | 552 | 10 | 3′ spacer 7 | 1899 | 13 | ++++ | + | + | ++ |
| 5′ spacer 8 | 552 | 10 | 3′ spacer 2 | 1399 | 12 | ++++ | + | + | ++ |
| 5′ spacer 8 | 553 | 13 | 3′ spacer 8 | 1916 | 10 | ++++ | + | + | ++ |
| 5′ spacer 8 | 553 | 13 | 3′ spacer 9 | 1934 | 10 | ++++ | + | + | ++ |
| 5′ spacer 8 | 553 | 13 | 3′ spacer 9 | 1935 | 12 | ++++ | + | + | ++ |
| 5′ spacer 8 | 553 | 13 | 3′ spacer 10 | 1952 | 10 | ++++ | + | + | +++ |
| 5′ spacer 8 | 553 | 13 | 3′ spacer 10 | 1953 | 12 | ++++ | + | + | +++ |
| 5′ spacer 8 | 553 | 13 | 3′ spacer 11 | 2279 | 10 | +++ | + | + | ++ |
| 5′ spacer 8 | 553 | 13 | 3′ spacer 11 | 2280 | 12 | ++++ | + | + | ++ |
| 5′ spacer 8 | 553 | 13 | 3′ spacer 16 | 2369 | 10 | ++++ | + | + | ++ |
| 5′ spacer 8 | 553 | 13 | 3′ spacer 12 | 2297 | 10 | +++ | + | NA | +++ |
| 5′ spacer 8 | 553 | 13 | 3′ spacer 12 | 2298 | 12 | ++++ | + | NA | ++ |
| 5′ spacer 8 | 553 | 13 | 3′ spacer 17 | 2689 | 10 | ++++ | + | NA | ++ |
| 5′ spacer 8 | 553 | 13 | 3′ spacer 17 | 2690 | 12 | ++++ | + | NA | ++ |
| 5′ spacer 8 | 553 | 13 | 3′ spacer 5 | 1543 | 12 | ++++ | + | NA | ++ |
| 5′ spacer 8 | 553 | 13 | 3′ spacer 13 | 2315 | 10 | ++++ | + | NA | ++ |
| 5′ spacer 8 | 553 | 13 | 3′ spacer 13 | 2316 | 12 | +++ | + | NA | + |
| 5′ spacer 8 | 553 | 13 | 3′ spacer 14 | 2333 | 10 | ++++ | + | NA | + |
| 5′ spacer 8 | 553 | 13 | 3′ spacer 14 | 2334 | 12 | ++++ | + | NA | + |
| 5′ spacer 8 | 553 | 13 | 3′ spacer 6 | 1880 | 11 | ++++ | + | + | + |
| 5′ spacer 8 | 553 | 13 | 3′ spacer 6 | 1881 | 13 | ++++ | + | + | + |
| 5′ spacer 8 | 553 | 13 | 3′ spacer 15 | 2352 | 12 | ++++ | + | + | + |
| 5′ spacer 8 | 553 | 13 | 3′ spacer 7 | 1898 | 11 | ++++ | + | + | ++ |
| 5′ spacer 8 | 553 | 13 | 3′ spacer 7 | 1899 | 13 | ++++ | + | + | ++ |
| 5′ spacer 8 | 553 | 13 | 3′ spacer 2 | 1399 | 12 | ++++ | + | + | ++ |
| 5′ spacer 9 | 570 | 10 | 3′ spacer 8 | 1916 | 10 | ++++ | + | + | ++ |
| 5′ spacer 9 | 570 | 10 | 3′ spacer 9 | 1934 | 10 | ++++ | + | + | ++ |
| 5′ spacer 9 | 570 | 10 | 3′ spacer 9 | 1935 | 12 | ++++ | + | + | ++ |
| 5′ spacer 9 | 570 | 10 | 3′ spacer 10 | 1952 | 10 | ++ | NA | + | ++ |
| 5′ spacer 9 | 570 | 10 | 3′ spacer 10 | 1953 | 12 | ++ | + | + | +++ |
| 5′ spacer 9 | 570 | 10 | 3′ spacer 11 | 2279 | 10 | + | + | + | ++ |
| 5′ spacer 9 | 570 | 10 | 3′ spacer 11 | 2280 | 12 | ++ | + | + | ++ |
| 5′ spacer 9 | 570 | 10 | 3′ spacer 16 | 2369 | 10 | ++++ | + | + | ++ |
| 5′ spacer 9 | 570 | 10 | 3′ spacer 12 | 2297 | 10 | +++ | + | NA | ++ |
| 5′ spacer 9 | 570 | 10 | 3′ spacer 12 | 2298 | 12 | ++++ | + | NA | + |
| 5′ spacer 9 | 570 | 10 | 3′ spacer 17 | 2689 | 10 | ++++ | + | NA | ++ |
| 5′ spacer 9 | 570 | 10 | 3′ spacer 17 | 2690 | 12 | ++++ | + | NA | ++ |
| 5′ spacer 9 | 570 | 10 | 3′ spacer 5 | 1543 | 12 | ++++ | + | NA | ++ |
| 5′ spacer 9 | 570 | 10 | 3′ spacer 13 | 2315 | 10 | ++ | + | NA | + |
| 5′ spacer 9 | 570 | 10 | 3′ spacer 13 | 2316 | 12 | ++ | + | NA | + |
| 5′ spacer 9 | 570 | 10 | 3′ spacer 14 | 2333 | 10 | ++++ | + | NA | + |
| 5′ spacer 9 | 570 | 10 | 3′ spacer 14 | 2334 | 12 | +++ | + | NA | + |
| 5′ spacer 9 | 570 | 10 | 3′ spacer 6 | 1880 | 11 | ++++ | + | + | + |
| 5′ spacer 9 | 570 | 10 | 3′ spacer 6 | 1881 | 13 | ++++ | + | + | + |
| 5′ spacer 9 | 570 | 10 | 3′ spacer 15 | 2352 | 12 | +++ | + | + | + |
| 5′ spacer 9 | 570 | 10 | 3′ spacer 7 | 1898 | 11 | ++++ | + | + | + |
| 5′ spacer 9 | 570 | 10 | 3′ spacer 7 | 1899 | 13 | ++++ | + | + | + |
| 5′ spacer 9 | 570 | 10 | 3′ spacer 2 | 1399 | 12 | +++ | + | + | + |
| 5′ spacer 9 | 571 | 12 | 3′ spacer 8 | 1916 | 10 | ++++ | + | + | ++ |
| 5′ spacer 9 | 571 | 12 | 3′ spacer 9 | 1934 | 10 | ++++ | + | + | ++ |
| 5′ spacer 9 | 571 | 12 | 3′ spacer 9 | 1935 | 12 | ++++ | + | + | ++ |
| 5′ spacer 9 | 571 | 12 | 3′ spacer 10 | 1952 | 10 | ++ | NA | + | ++ |
| 5′ spacer 9 | 571 | 12 | 3′ spacer 10 | 1953 | 12 | ++ | + | + | ++ |
| 5′ spacer 9 | 571 | 12 | 3′ spacer 11 | 2279 | 10 | ++ | + | + | ++ |
| 5′ spacer 9 | 571 | 12 | 3′ spacer 11 | 2280 | 12 | +++ | + | + | ++ |
| 5′ spacer 9 | 571 | 12 | 3′ spacer 16 | 2369 | 10 | ++++ | + | + | ++ |
| 5′ spacer 9 | 571 | 12 | 3′ spacer 12 | 2297 | 10 | +++ | + | NA | + |
| 5′ spacer 9 | 571 | 12 | 3′ spacer 12 | 2298 | 12 | ++++ | + | NA | ++ |
| 5′ spacer 9 | 571 | 12 | 3′ spacer 17 | 2689 | 10 | ++++ | + | NA | ++ |
| 5′ spacer 9 | 571 | 12 | 3′ spacer 17 | 2690 | 12 | ++++ | + | NA | ++ |
| 5′ spacer 9 | 571 | 12 | 3′ spacer 5 | 1543 | 12 | ++++ | + | NA | ++ |
| 5′ spacer 9 | 571 | 12 | 3′ spacer 13 | 2315 | 10 | + | + | NA | + |
| 5′ spacer 9 | 571 | 12 | 3′ spacer 13 | 2316 | 12 | ++ | + | NA | + |
| 5′ spacer 9 | 571 | 12 | 3′ spacer 14 | 2333 | 10 | ++++ | + | NA | + |
| 5′ spacer 9 | 571 | 12 | 3′ spacer 14 | 2334 | 12 | ++++ | + | NA | + |
| 5′ spacer 9 | 571 | 12 | 3′ spacer 6 | 1880 | 11 | ++++ | + | + | + |
| 5′ spacer 9 | 571 | 12 | 3′ spacer 6 | 1881 | 13 | ++++ | + | + | + |
| 5′ spacer 9 | 571 | 12 | 3′ spacer 15 | 2352 | 12 | +++ | + | + | + |
| 5′ spacer 9 | 571 | 12 | 3′ spacer 7 | 1898 | 11 | ++++ | + | + | + |
| 5′ spacer 9 | 571 | 12 | 3′ spacer 7 | 1899 | 13 | ++++ | + | + | + |
| 5′ spacer 9 | 571 | 12 | 3′ spacer 2 | 1399 | 12 | +++ | + | + | + |

A total of twenty-eight (28) 5′ PEgRNAs and twenty-five (25) 3′ PEgRNAs were screened. All 5′ PEgRNAs comprised an RTT of SEQ ID NO: 2691 (RTT pairing NO: 1). All 3′ PEgRNAs comprised an RTT of SEQ ID NO: 3075 (RTT pairing NO: 1). The 5′ PEgRNA spacers that were screened were 5′ spacers 3 and 7-19 (SEQ ID NOs: 39, 518, 536, 554, 572, 590, 912, 929, 947, 965, 983, 1001, 1019, and 1341). The 3′ PEgRNA spacers that were screened were 3′ spacers 2 and 5-17 (SEQ ID NOs: 1381, 1525, 1864, 1882, 1900, 1918, 1936, 2263, 2281, 2299, 2317, 2335, 2353, and 2673). The 5′ PEgRNA and 3′ PEgRNA PBS lengths were 10, 11, 12 or 13 nucleotides as indicated in Table 52. Successful dual prime editing was observed in all PEgRNA pairs examined.

Table 53 summarizes 5′ spacers tested in Examples 2 and 3. Table 54 summarizes 3′ spacers tested in Examples 2 and 3. The Nick-to-repeat distance indicates the distance from the nick generated by the PEgRNA to the end of the CTG repeats, where in the end of the CTG repeats is the end proximal to the nick.

TABLE 53
5′ spacers tested in Examples 2 and 3

| Spacer | SEQ | Nick-to-repeat |
|---|---|---|
| No. | ID NO: | distance |
| 5′ spacer1 | 1 | 7 |
| 5′ spacer3 | 39 | 50 |
| 5′ spacer4 | 58 | 70 |
| 5′ spacer5 | 77 | 172 |
| 5′ spacer6 | 484 | 199 |
| 5′ spacer7 | 518 | 200 |
| 5′ spacer8 | 536 | 173 |
| 5′ spacer9 | 554 | 167 |
| 5′ spacer10 | 572 | 117 |
| 5′ spacer11 | 590 | 85 |
| 5′ spacer12 | 912 | 22 |
| 5′ spacer13 | 929 | 247 |
| 5′ spacer14 | 947 | 190 |
| 5′ spacer15 | 965 | 66 |
| 5′ spacer16 | 983 | 56 |
| 5′ spacer17 | 1001 | 39 |
| 5′ spacer18 | 1019 | 30 |
| 5′ spacer19 | 1341 | 8 |

TABLE 54
3′ spacers tested in Examples 2 and 3

| | SEQ | Nick-to-repeat |
|---|---|---|
| Spacer No. | ID NO: | distance |
| 3′ spacer 1 | 1359 | 47 |
| 3′ spacer 2 | 1381 | 96 |
| 3′ spacer 3 | 1403 | 127 |
| 3′ spacer 4 | 1503 | 239 |
| 3′ spacer 5 | 1525 | 305 |
| 3′ spacer 6 | 1864 | 36 |
| 3′ spacer 7 | 1882 | 91 |
| 3′ spacer 8 | 1900 | 100 |
| 3′ spacer 9 | 1918 | 122 |
| 3′ spacer 10 | 1936 | 171 |
| 3′ spacer 11 | 2263 | 204 |
| 3′ spacer 12 | 2281 | 259 |
| 3′ spacer 13 | 2299 | 333 |
| 3′ spacer 14 | 2317 | 342 |
| 3′ spacer 15 | 2335 | 74 |
| 3′ spacer 16 | 2353 | 231 |
| 3′ spacer 17 | 2673 | 289 |

### Example 4. Dual Prime Editing for Excision of CTG Repeats Associated with DM1 without Insertion

PEgRNA pairs were designed to remove the CTG repeats in the DMPK gene without insertion of any exogenous sequence. PEgRNAs were assembled as described in Example 1. Wild type HEK 293T cells were transfected with polynucleotides encoding a prime editor fusion protein and a pair of PEgRNA in arrayed 96-well plates for assessment of editing by high-throughput sequencing. Three days after transfection, gDNA was harvested in lysis buffer for high throughput sequencing and sequenced using MiSeq. Editing efficiency is reflected by desired excision of the CTG repeats. Editing efficiency and indel frequency were assessed with Version 1 of CRISPResso2 dual-flap analysis, as described in Example 3.

The results are summarized in Table 55. In Table 55, each row presents information for a pair of PEgRNAs (i.e., a 5′ PEgRNA and a 3′ PEgRNA) used in dual prime editing. Each PEgRNA is identified by a sequence identifier (“SEQ ID NO”), and information is also presented for the RTT length and PBS length as well as the RTT Pairing number for each PEgRNA. The RTT Paring number assigned to each PEgRNA indicates the spacer number of the pairing PEgRNA, as provided in Tables 39 and 40. A total of fifteen (15) 5′ PEgRNAs and fifteen (15) 3′ PEgRNAs were screened. All 5′ PEgRNAs comprised a spacer of SEQ ID NO: 484 (Paring number for 3′ RTT: 385). All 3′ PEgRNAs comprised a spacer of SEQ ID NO: 1525 (Paring number for 5′ RTT: 389). Different 5′ PEgRNA and 3′ PEgRNA PBS lengths 8, 10, 12, 14, and 17, were tested as shown in Table 55. The RTT length in this experiment reflects the length of region of complementarity between a RTT and the endogenous DMPK gene sequence. For example, the 5′ RTT length in this experiment reflects the region of complementarity between the 5′ RTT and the DMPK strand having the search target sequence of the 3′ PEgRA (specifically, the DMPK strand having the sequence 3′ to nucleotide 4 of the search target of the 3′ PEgRNA). The 3′ RTT length in this experiment reflects the region of complementarity between the 3′ RTT and the DMPK strand having the search target sequence of the 5′ PEgRA (specifically, the DMPK strand having the sequence 3′ to nucleotide 4 of the search target of the 3′ PEgRNA). Variant 5′ PEgRNA and 3′ PEgRNA RTT lengths 20, 25, and 30 nucleotides were tested as shown in Table 55.

The following scale was used for editing efficiency: PEgRNA pairs exhibiting editing efficiency of 0% are indicated with “−”, PEgRNA pairs exhibiting editing efficiency above 0% and up to 25% are indicated with “+”, PEgRNA pairs exhibiting editing efficiency above 25% and up to 50% are indicated with “++”, PEgRNA pairs exhibiting editing efficiency above 50% and up to 75% are indicated with “+++”, and PEgRNA pairs exhibiting editing efficiency above 75% and up to 100% are indicated with “++++”. The following scale was used for indel rates: PEgRNA pairs exhibiting indel rates of 0% are indicated with “−”, PEgRNA pairs exhibiting indel rates above 0% and up to 5% are indicated with “+”, PEgRNA pairs exhibiting indel rates above 5% and up to 25% are indicated with “++”, PEgRNA pairs exhibiting indel rates above 25% and up to 50% are indicated with “+++”, and PEgRNA pairs exhibiting indel rates above 50% are indicated with “++++”. Successful dual prime editing resulting in deletion of CTG repeats was observed in all PEgRNA pairs tested.

TABLE 55
Dual prime editing for CTG excision without insertion in HEK 293T cells.

| 5′ | 5′ | 5′ | 5′ | 3′ | 3′ | 3′ | 3′ | | | | |
|---|---|---|---|---|---|---|---|---|---|---|---|
| pegRNA | pegRNA | pegRNA | RTT | pegRNA | pegRNA | pegRNA | RTT | | | | % indel |
| SEQ ID | PBS | RTT | paring | SEQ ID | PBS | RTT | paring | % editing | % indel | % indel | nick to |
| NO: | length | length | No. | NO: | length | length | No. | efficiency | Forward | Reverse | nick |
| 504 | 10 | 20 | 385 | 1549 | 10 | 20 | 389 | ++ | + | + | ++ |
| 504 | 10 | 20 | 385 | 1554 | 10 | 25 | 389 | ++ | + | + | + |
| 504 | 10 | 20 | 385 | 1559 | 10 | 30 | 389 | + | + | + | ++ |
| 504 | 10 | 20 | 385 | 1550 | 12 | 20 | 389 | ++ | + | + | + |
| 504 | 10 | 20 | 385 | 1555 | 12 | 25 | 389 | ++++ | ++ | + | ++ |
| 504 | 10 | 20 | 385 | 1560 | 12 | 30 | 389 | ++ | ++ | + | ++ |
| 504 | 10 | 20 | 385 | 1551 | 14 | 20 | 389 | + | + | + | + |
| 504 | 10 | 20 | 385 | 1556 | 14 | 25 | 389 | ++++ | ++ | + | ++ |
| 504 | 10 | 20 | 385 | 1561 | 14 | 30 | 389 | + | + | + | + |
| 504 | 10 | 20 | 385 | 1552 | 17 | 20 | 389 | ++ | + | + | + |
| 504 | 10 | 20 | 385 | 1557 | 17 | 25 | 389 | + | + | + | + |
| 504 | 10 | 20 | 385 | 1562 | 17 | 30 | 389 | ++ | + | + | + |
| 504 | 10 | 20 | 385 | 1548 | 8 | 20 | 389 | ++++ | + | + | + |
| 504 | 10 | 20 | 385 | 1553 | 8 | 25 | 389 | ++ | + | + | ++ |
| 504 | 10 | 20 | 385 | 1558 | 8 | 30 | 389 | +++ | + | + | + |
| 509 | 10 | 25 | 385 | 1549 | 10 | 20 | 389 | ++ | +++ | + | +++ |
| 509 | 10 | 25 | 385 | 1554 | 10 | 25 | 389 | ++ | + | + | + |
| 509 | 10 | 25 | 385 | 1559 | 10 | 30 | 389 | + | ++ | + | ++ |
| 509 | 10 | 25 | 385 | 1550 | 12 | 20 | 389 | + | ++ | + | ++ |
| 509 | 10 | 25 | 385 | 1555 | 12 | 25 | 389 | ++++ | ++ | + | ++ |
| 509 | 10 | 25 | 385 | 1560 | 12 | 30 | 389 | ++ | + | + | + |
| 509 | 10 | 25 | 385 | 1551 | 14 | 20 | 389 | + | ++ | + | ++ |
| 509 | 10 | 25 | 385 | 1556 | 14 | 25 | 389 | +++ | + | + | + |
| 509 | 10 | 25 | 385 | 1561 | 14 | 30 | 389 | + | + | + | + |
| 509 | 10 | 25 | 385 | 1552 | 17 | 20 | 389 | + | + | + | + |
| 509 | 10 | 25 | 385 | 1557 | 17 | 25 | 389 | + | ++ | + | ++ |
| 509 | 10 | 25 | 385 | 1562 | 17 | 30 | 389 | +++ | + | + | + |
| 509 | 10 | 25 | 385 | 1548 | 8 | 20 | 389 | ++ | ++ | + | ++ |
| 509 | 10 | 25 | 385 | 1553 | 8 | 25 | 389 | ++ | + | + | + |
| 509 | 10 | 25 | 385 | 1558 | 8 | 30 | 389 | +++ | ++ | + | ++ |
| 514 | 10 | 30 | 385 | 1549 | 10 | 20 | 389 | +++ | + | + | + |
| 514 | 10 | 30 | 385 | 1554 | 10 | 25 | 389 | ++++ | + | + | + |
| 514 | 10 | 30 | 385 | 1559 | 10 | 30 | 389 | ++ | + | + | ++ |
| 514 | 10 | 30 | 385 | 1550 | 12 | 20 | 389 | +++ | +++ | + | +++ |
| 514 | 10 | 30 | 385 | 1555 | 12 | 25 | 389 | ++++ | + | + | + |
| 514 | 10 | 30 | 385 | 1560 | 12 | 30 | 389 | +++ | + | + | + |
| 514 | 10 | 30 | 385 | 1551 | 14 | 20 | 389 | ++ | +++ | + | +++ |
| 514 | 10 | 30 | 385 | 1556 | 14 | 25 | 389 | ++++ | + | + | + |
| 514 | 10 | 30 | 385 | 1561 | 14 | 30 | 389 | + | ++ | + | ++ |
| 514 | 10 | 30 | 385 | 1552 | 17 | 20 | 389 | ++ | +++ | − | +++ |
| 514 | 10 | 30 | 385 | 1557 | 17 | 25 | 389 | ++ | + | + | + |
| 514 | 10 | 30 | 385 | 1562 | 17 | 30 | 389 | ++++ | + | + | + |
| 514 | 10 | 30 | 385 | 1548 | 8 | 20 | 389 | ++++ | + | + | + |
| 514 | 10 | 30 | 385 | 1553 | 8 | 25 | 389 | +++ | ++++ | + | ++++ |
| 514 | 10 | 30 | 385 | 1558 | 8 | 30 | 389 | ++++ | + | + | + |
| 505 | 12 | 20 | 385 | 1549 | 10 | 20 | 389 | +++ | + | + | ++ |
| 505 | 12 | 20 | 385 | 1554 | 10 | 25 | 389 | ++ | ++ | + | ++ |
| 505 | 12 | 20 | 385 | 1559 | 10 | 30 | 389 | + | + | + | + |
| 505 | 12 | 20 | 385 | 1550 | 12 | 20 | 389 | ++ | + | + | + |
| 505 | 12 | 20 | 385 | 1555 | 12 | 25 | 389 | ++++ | ++ | + | ++ |
| 505 | 12 | 20 | 385 | 1560 | 12 | 30 | 389 | ++ | + | + | + |
| 505 | 12 | 20 | 385 | 1551 | 14 | 20 | 389 | + | ++ | + | ++ |
| 505 | 12 | 20 | 385 | 1556 | 14 | 25 | 389 | ++++ | + | + | + |
| 505 | 12 | 20 | 385 | 1561 | 14 | 30 | 389 | + | ++ | + | ++ |
| 505 | 12 | 20 | 385 | 1552 | 17 | 20 | 389 | + | + | + | + |
| 505 | 12 | 20 | 385 | 1557 | 17 | 25 | 389 | + | + | + | + |
| 505 | 12 | 20 | 385 | 1562 | 17 | 30 | 389 | ++ | + | + | + |
| 505 | 12 | 20 | 385 | 1548 | 8 | 20 | 389 | ++++ | + | + | + |
| 505 | 12 | 20 | 385 | 1553 | 8 | 25 | 389 | +++ | + | + | + |
| 505 | 12 | 20 | 385 | 1558 | 8 | 30 | 389 | ++ | + | + | ++ |
| 510 | 12 | 25 | 385 | 1549 | 10 | 20 | 389 | ++ | ++ | + | ++ |
| 510 | 12 | 25 | 385 | 1554 | 10 | 25 | 389 | + | + | + | + |
| 510 | 12 | 25 | 385 | 1559 | 10 | 30 | 389 | ++ | ++ | + | ++ |
| 510 | 12 | 25 | 385 | 1550 | 12 | 20 | 389 | ++ | + | + | + |
| 510 | 12 | 25 | 385 | 1555 | 12 | 25 | 389 | ++++ | + | + | + |
| 510 | 12 | 25 | 385 | 1560 | 12 | 30 | 389 | +++ | + | + | ++ |
| 510 | 12 | 25 | 385 | 1551 | 14 | 20 | 389 | ++ | + | + | + |
| 510 | 12 | 25 | 385 | 1556 | 14 | 25 | 389 | +++ | + | + | + |
| 510 | 12 | 25 | 385 | 1561 | 14 | 30 | 389 | + | + | + | + |
| 510 | 12 | 25 | 385 | 1552 | 17 | 20 | 389 | + | ++ | + | ++ |
| 510 | 12 | 25 | 385 | 1557 | 17 | 25 | 389 | + | + | + | + |
| 510 | 12 | 25 | 385 | 1562 | 17 | 30 | 389 | ++ | + | + | + |
| 510 | 12 | 25 | 385 | 1548 | 8 | 20 | 389 | + | + | ++ | ++ |
| 510 | 12 | 25 | 385 | 1553 | 8 | 25 | 389 | ++ | + | ++ | ++ |
| 510 | 12 | 25 | 385 | 1558 | 8 | 30 | 389 | +++ | + | + | + |
| 515 | 12 | 30 | 385 | 1549 | 10 | 20 | 389 | ++ | + | + | + |
| 515 | 12 | 30 | 385 | 1554 | 10 | 25 | 389 | ++ | + | + | + |
| 515 | 12 | 30 | 385 | 1559 | 10 | 30 | 389 | ++ | ++ | + | ++ |
| 515 | 12 | 30 | 385 | 1550 | 12 | 20 | 389 | ++ | + | + | + |
| 515 | 12 | 30 | 385 | 1555 | 12 | 25 | 389 | ++++ | + | + | + |
| 515 | 12 | 30 | 385 | 1560 | 12 | 30 | 389 | + | + | + | + |
| 515 | 12 | 30 | 385 | 1551 | 14 | 20 | 389 | ++ | + | + | + |
| 515 | 12 | 30 | 385 | 1556 | 14 | 25 | 389 | ++++ | + | + | + |
| 515 | 12 | 30 | 385 | 1561 | 14 | 30 | 389 | ++ | + | + | + |
| 515 | 12 | 30 | 385 | 1552 | 17 | 20 | 389 | ++ | + | + | + |
| 515 | 12 | 30 | 385 | 1557 | 17 | 25 | 389 | + | ++ | + | ++ |
| 515 | 12 | 30 | 385 | 1562 | 17 | 30 | 389 | + | + | − | + |
| 515 | 12 | 30 | 385 | 1548 | 8 | 20 | 389 | + | + | + | + |
| 515 | 12 | 30 | 385 | 1553 | 8 | 25 | 389 | ++ | + | + | + |
| 515 | 12 | 30 | 385 | 1558 | 8 | 30 | 389 | ++ | + | + | + |
| 506 | 14 | 20 | 385 | 1549 | 10 | 20 | 389 | + | + | + | + |
| 506 | 14 | 20 | 385 | 1554 | 10 | 25 | 389 | ++ | + | + | + |
| 506 | 14 | 20 | 385 | 1559 | 10 | 30 | 389 | + | ++ | + | ++ |
| 506 | 14 | 20 | 385 | 1550 | 12 | 20 | 389 | +++ | + | + | + |
| 506 | 14 | 20 | 385 | 1555 | 12 | 25 | 389 | ++++ | + | + | + |
| 506 | 14 | 20 | 385 | 1560 | 12 | 30 | 389 | ++ | + | + | + |
| 506 | 14 | 20 | 385 | 1551 | 14 | 20 | 389 | ++ | +++ | + | +++ |
| 506 | 14 | 20 | 385 | 1556 | 14 | 25 | 389 | ++++ | ++ | + | +++ |
| 506 | 14 | 20 | 385 | 1561 | 14 | 30 | 389 | ++ | + | + | + |
| 506 | 14 | 20 | 385 | 1552 | 17 | 20 | 389 | + | ++ | + | ++ |
| 506 | 14 | 20 | 385 | 1557 | 17 | 25 | 389 | + | + | + | + |
| 506 | 14 | 20 | 385 | 1562 | 17 | 30 | 389 | ++ | ++ | + | ++ |
| 506 | 14 | 20 | 385 | 1548 | 8 | 20 | 389 | +++ | ++ | + | ++ |
| 506 | 14 | 20 | 385 | 1553 | 8 | 25 | 389 | +++ | + | + | + |
| 506 | 14 | 20 | 385 | 1558 | 8 | 30 | 389 | ++ | + | + | + |
| 511 | 14 | 25 | 385 | 1549 | 10 | 20 | 389 | + | + | + | ++ |
| 511 | 14 | 25 | 385 | 1554 | 10 | 25 | 389 | ++ | ++ | + | ++ |
| 511 | 14 | 25 | 385 | 1559 | 10 | 30 | 389 | ++ | + | + | + |
| 511 | 14 | 25 | 385 | 1550 | 12 | 20 | 389 | + | + | + | + |
| 511 | 14 | 25 | 385 | 1555 | 12 | 25 | 389 | ++++ | + | + | + |
| 511 | 14 | 25 | 385 | 1560 | 12 | 30 | 389 | ++ | + | + | + |
| 511 | 14 | 25 | 385 | 1551 | 14 | 20 | 389 | ++ | + | + | + |
| 511 | 14 | 25 | 385 | 1556 | 14 | 25 | 389 | ++ | + | + | + |
| 511 | 14 | 25 | 385 | 1561 | 14 | 30 | 389 | + | ++ | + | ++ |
| 511 | 14 | 25 | 385 | 1552 | 17 | 20 | 389 | + | + | ++ | ++ |
| 511 | 14 | 25 | 385 | 1557 | 17 | 25 | 389 | + | + | + | + |
| 511 | 14 | 25 | 385 | 1562 | 17 | 30 | 389 | ++ | + | + | + |
| 511 | 14 | 25 | 385 | 1548 | 8 | 20 | 389 | + | ++ | ++ | ++ |
| 511 | 14 | 25 | 385 | 1553 | 8 | 25 | 389 | ++ | + | + | + |
| 511 | 14 | 25 | 385 | 1558 | 8 | 30 | 389 | ++ | + | + | ++ |
| 516 | 14 | 30 | 385 | 1549 | 10 | 20 | 389 | ++ | ++ | + | ++ |
| 516 | 14 | 30 | 385 | 1554 | 10 | 25 | 389 | ++ | ++ | + | ++ |
| 516 | 14 | 30 | 385 | 1559 | 10 | 30 | 389 | ++ | ++ | + | ++ |
| 516 | 14 | 30 | 385 | 1550 | 12 | 20 | 389 | ++ | + | + | + |
| 516 | 14 | 30 | 385 | 1555 | 12 | 25 | 389 | ++++ | + | + | + |
| 516 | 14 | 30 | 385 | 1560 | 12 | 30 | 389 | ++ | + | + | + |
| 516 | 14 | 30 | 385 | 1551 | 14 | 20 | 389 | ++ | + | + | + |
| 516 | 14 | 30 | 385 | 1556 | 14 | 25 | 389 | ++++ | + | + | + |
| 516 | 14 | 30 | 385 | 1561 | 14 | 30 | 389 | + | ++ | + | ++ |
| 516 | 14 | 30 | 385 | 1552 | 17 | 20 | 389 | ++ | + | + | + |
| 516 | 14 | 30 | 385 | 1557 | 17 | 25 | 389 | ++ | + | + | + |
| 516 | 14 | 30 | 385 | 1562 | 17 | 30 | 389 | ++ | + | + | + |
| 516 | 14 | 30 | 385 | 1548 | 8 | 20 | 389 | ++++ | + | + | + |
| 516 | 14 | 30 | 385 | 1553 | 8 | 25 | 389 | ++ | + | + | + |
| 516 | 14 | 30 | 385 | 1558 | 8 | 30 | 389 | +++ | + | + | + |
| 507 | 17 | 20 | 385 | 1549 | 10 | 20 | 389 | +++ | ++ | + | ++ |
| 507 | 17 | 20 | 385 | 1554 | 10 | 25 | 389 | ++ | ++ | + | ++ |
| 507 | 17 | 20 | 385 | 1559 | 10 | 30 | 389 | + | ++ | + | ++ |
| 507 | 17 | 20 | 385 | 1550 | 12 | 20 | 389 | + | + | + | + |
| 507 | 17 | 20 | 385 | 1555 | 12 | 25 | 389 | ++++ | + | + | + |
| 507 | 17 | 20 | 385 | 1560 | 12 | 30 | 389 | ++ | ++ | + | ++ |
| 507 | 17 | 20 | 385 | 1551 | 14 | 20 | 389 | + | ++ | + | ++ |
| 507 | 17 | 20 | 385 | 1556 | 14 | 25 | 389 | +++ | ++ | + | ++ |
| 507 | 17 | 20 | 385 | 1561 | 14 | 30 | 389 | + | + | + | + |
| 507 | 17 | 20 | 385 | 1552 | 17 | 20 | 389 | + | ++++ | + | ++++ |
| 507 | 17 | 20 | 385 | 1557 | 17 | 25 | 389 | + | ++ | + | ++ |
| 507 | 17 | 20 | 385 | 1562 | 17 | 30 | 389 | + | + | + | + |
| 507 | 17 | 20 | 385 | 1548 | 8 | 20 | 389 | ++++ | ++ | + | ++ |
| 507 | 17 | 20 | 385 | 1553 | 8 | 25 | 389 | ++ | + | + | + |
| 507 | 17 | 20 | 385 | 1558 | 8 | 30 | 389 | ++ | + | + | + |
| 512 | 17 | 25 | 385 | 1549 | 10 | 20 | 389 | + | + | + | + |
| 512 | 17 | 25 | 385 | 1554 | 10 | 25 | 389 | ++ | + | + | + |
| 512 | 17 | 25 | 385 | 1559 | 10 | 30 | 389 | ++ | + | + | + |
| 512 | 17 | 25 | 385 | 1550 | 12 | 20 | 389 | + | ++ | + | ++ |
| 512 | 17 | 25 | 385 | 1555 | 12 | 25 | 389 | ++++ | ++ | + | ++ |
| 512 | 17 | 25 | 385 | 1560 | 12 | 30 | 389 | +++ | + | + | + |
| 512 | 17 | 25 | 385 | 1551 | 14 | 20 | 389 | + | + | + | + |
| 512 | 17 | 25 | 385 | 1556 | 14 | 25 | 389 | +++ | ++ | + | ++ |
| 512 | 17 | 25 | 385 | 1561 | 14 | 30 | 389 | ++ | + | + | + |
| 512 | 17 | 25 | 385 | 1552 | 17 | 20 | 389 | + | ++ | + | ++ |
| 512 | 17 | 25 | 385 | 1557 | 17 | 25 | 389 | + | + | + | + |
| 512 | 17 | 25 | 385 | 1562 | 17 | 30 | 389 | ++ | + | + | + |
| 512 | 17 | 25 | 385 | 1548 | 8 | 20 | 389 | ++++ | + | + | + |
| 512 | 17 | 25 | 385 | 1553 | 8 | 25 | 389 | + | + | + | + |
| 512 | 17 | 25 | 385 | 1558 | 8 | 30 | 389 | ++ | + | + | + |
| 517 | 17 | 30 | 385 | 1549 | 10 | 20 | 389 | + | ++ | + | ++ |
| 517 | 17 | 30 | 385 | 1554 | 10 | 25 | 389 | ++ | ++ | + | ++ |
| 517 | 17 | 30 | 385 | 1559 | 10 | 30 | 389 | + | + | + | + |
| 517 | 17 | 30 | 385 | 1550 | 12 | 20 | 389 | + | + | + | + |
| 517 | 17 | 30 | 385 | 1555 | 12 | 25 | 389 | ++++ | ++ | + | ++ |
| 517 | 17 | 30 | 385 | 1560 | 12 | 30 | 389 | + | ++ | + | ++ |
| 517 | 17 | 30 | 385 | 1551 | 14 | 20 | 389 | + | + | + | + |
| 517 | 17 | 30 | 385 | 1556 | 14 | 25 | 389 | +++ | + | + | + |
| 517 | 17 | 30 | 385 | 1561 | 14 | 30 | 389 | + | + | + | + |
| 517 | 17 | 30 | 385 | 1552 | 17 | 20 | 389 | + | − | ++ | ++ |
| 517 | 17 | 30 | 385 | 1557 | 17 | 25 | 389 | + | ++ | + | ++ |
| 517 | 17 | 30 | 385 | 1562 | 17 | 30 | 389 | + | + | + | ++ |
| 517 | 17 | 30 | 385 | 1548 | 8 | 20 | 389 | + | +++ | + | +++ |
| 517 | 17 | 30 | 385 | 1553 | 8 | 25 | 389 | ++ | ++ | + | ++ |
| 517 | 17 | 30 | 385 | 1558 | 8 | 30 | 389 | ++ | + | + | + |
| 503 | 8 | 20 | 385 | 1549 | 10 | 20 | 389 | ++ | + | + | + |
| 503 | 8 | 20 | 385 | 1554 | 10 | 25 | 389 | ++ | + | + | + |
| 503 | 8 | 20 | 385 | 1559 | 10 | 30 | 389 | + | ++ | + | ++ |
| 503 | 8 | 20 | 385 | 1550 | 12 | 20 | 389 | ++ | ++ | + | ++ |
| 503 | 8 | 20 | 385 | 1555 | 12 | 25 | 389 | ++++ | + | + | + |
| 503 | 8 | 20 | 385 | 1560 | 12 | 30 | 389 | ++ | ++ | + | ++ |
| 503 | 8 | 20 | 385 | 1551 | 14 | 20 | 389 | +++ | + | + | + |
| 503 | 8 | 20 | 385 | 1556 | 14 | 25 | 389 | ++++ | + | + | + |
| 503 | 8 | 20 | 385 | 1561 | 14 | 30 | 389 | + | ++ | + | ++ |
| 503 | 8 | 20 | 385 | 1552 | 17 | 20 | 389 | +++ | +++ | + | +++ |
| 503 | 8 | 20 | 385 | 1557 | 17 | 25 | 389 | + | ++ | + | ++ |
| 503 | 8 | 20 | 385 | 1562 | 17 | 30 | 389 | ++ | + | + | + |
| 503 | 8 | 20 | 385 | 1548 | 8 | 20 | 389 | ++++ | + | + | + |
| 503 | 8 | 20 | 385 | 1553 | 8 | 25 | 389 | + | + | + | + |
| 503 | 8 | 20 | 385 | 1558 | 8 | 30 | 389 | ++ | + | + | + |
| 508 | 8 | 25 | 385 | 1549 | 10 | 20 | 389 | ++ | ++ | + | ++ |
| 508 | 8 | 25 | 385 | 1554 | 10 | 25 | 389 | + | + | + | + |
| 508 | 8 | 25 | 385 | 1559 | 10 | 30 | 389 | + | + | + | + |
| 508 | 8 | 25 | 385 | 1550 | 12 | 20 | 389 | + | + | + | + |
| 508 | 8 | 25 | 385 | 1555 | 12 | 25 | 389 | ++++ | + | + | + |
| 508 | 8 | 25 | 385 | 1560 | 12 | 30 | 389 | +++ | + | + | + |
| 508 | 8 | 25 | 385 | 1551 | 14 | 20 | 389 | + | ++++ | + | ++++ |
| 508 | 8 | 25 | 385 | 1556 | 14 | 25 | 389 | ++++ | + | + | + |
| 508 | 8 | 25 | 385 | 1561 | 14 | 30 | 389 | + | + | + | + |
| 508 | 8 | 25 | 385 | 1552 | 17 | 20 | 389 | + | + | + | + |
| 508 | 8 | 25 | 385 | 1557 | 17 | 25 | 389 | + | ++ | + | ++ |
| 508 | 8 | 25 | 385 | 1562 | 17 | 30 | 389 | +++ | + | + | ++ |
| 508 | 8 | 25 | 385 | 1548 | 8 | 20 | 389 | ++++ | + | + | + |
| 508 | 8 | 25 | 385 | 1553 | 8 | 25 | 389 | ++ | + | + | ++ |
| 508 | 8 | 25 | 385 | 1558 | 8 | 30 | 389 | +++ | + | + | + |
| 513 | 8 | 30 | 385 | 1549 | 10 | 20 | 389 | + | + | + | + |
| 513 | 8 | 30 | 385 | 1554 | 10 | 25 | 389 | +++ | + | + | + |
| 513 | 8 | 30 | 385 | 1559 | 10 | 30 | 389 | + | + | + | + |
| 513 | 8 | 30 | 385 | 1550 | 12 | 20 | 389 | + | + | + | ++ |
| 513 | 8 | 30 | 385 | 1555 | 12 | 25 | 389 | ++++ | + | + | + |
| 513 | 8 | 30 | 385 | 1560 | 12 | 30 | 389 | ++ | + | + | + |
| 513 | 8 | 30 | 385 | 1551 | 14 | 20 | 389 | + | + | + | + |
| 513 | 8 | 30 | 385 | 1556 | 14 | 25 | 389 | +++ | + | + | + |
| 513 | 8 | 30 | 385 | 1561 | 14 | 30 | 389 | + | + | + | + |
| 513 | 8 | 30 | 385 | 1552 | 17 | 20 | 389 | + | + | + | ++ |
| 513 | 8 | 30 | 385 | 1557 | 17 | 25 | 389 | + | + | + | + |
| 513 | 8 | 30 | 385 | 1562 | 17 | 30 | 389 | ++ | + | + | + |
| 513 | 8 | 30 | 385 | 1548 | 8 | 20 | 389 | ++++ | + | + | + |
| 513 | 8 | 30 | 385 | 1553 | 8 | 25 | 389 | ++++ | + | + | + |
| 513 | 8 | 30 | 385 | 1558 | 8 | 30 | 389 | +++ | + | ++ | ++ |

### Example 5. Dual Prime Editing for Excision of CTG Repeats in Myotonic Dystrophy Type 1 (DM1) Patient iPSC Cells

Based on the results as shown in Examples 2 and 3, 5′ PEgRNAs having a spacer identified by SEQ ID NO: 77, 484, 536, 590, 1019, or 1341 and 3′ PEgRNAs having a spacer identified by SEQ ID NO: 1525, 1900, 1936, 2263, 2281, 2299, 2353, or 2673 were selected for assessment in DM1 patient derived iPSC cells.

PEgRNAs were assembled as described in Example 1. iPSCs derived from myotonic dystrophy Type I patients were transfected with polynucleotides encoding a prime editor fusion protein and a pair of PEgRNAs as described in Example 1. Three days after transfection, gDNA was harvested in lysis buffer for sequencing and analysis with droplet digital PCR (ddPCR).

iPSC cells derived from three DM1 individuals were used. The three iPSC cell lines, iPSC1, iPSC2, and iPSC3, each carries 1600, 1150, and 238 CTG repeats in the DMPK gene, respectively (repeat numbers as reported by iPSC cell vendor). DMPK CTG repeat excision and attB integration with the dual prime editing system were assessed as follows: Reference samples were prepared by adding IDT gblock DNAs that contains desired edits into human gDNAs collected from healthy fibroblasts. The amount of edited reads was determined by a FAM primer set which includes a specific reverse primer targeting the RTT sequence that was introduced into the edited allele. The amount of total DMPK reads was determined by a HEX primer set which targets a different region of DMPK that is multiple kilobases away from the edited location. Editing efficiency for experimental samples was interpolated from a standard curve of reference samples containing different editing % (from 0 to 75%).

The results are summarized in Table 56. In Table 56, each row presents information for a pair of PEgRNAs (i.e., a 5′ PEgRNA and a 3′ PEgRNA) used in dual prime editing. Each PEgRNA is identified by a sequence identifier (“SEQ ID NO”), and information is also presented for the PBS length and spacer number of each PEgRNA that was tested. All 5′ PEgRNAs comprised an RTT of SEQ ID NO: 2691 (RTT pairing NO: 1). All 3′ PEgRNAs comprised an RTT of SEQ ID NO: 3075 (RTT pairing NO: 1). All 5′ PEgRNAs and 3′ PEgRNAs had a PBS length of 10 nt. A total of six (6) 5′ PEgRNAs and eight (8) 3′ PEgRNAs were tested. A total of six (6) 5′ spacers (SEQ ID NOs: 77, 484, 536, 590, 1019, and 1341) and eight (8) 3′ spacers (SEQ ID NOs: 1525, 1900, 1936, 2263, 2281, 2299, 2353, and 2673) were screened. The following scale was used for editing efficiency: PEgRNA pairs exhibiting editing efficiency above 0% and up to 25% are indicated with “+”, PEgRNA pairs exhibiting editing efficiency above 25% and up to 50% are indicated with “++”, PEgRNA pairs exhibiting editing efficiency above 50% and up to 75% are indicated with “+++”. Successful dual prime editing was observed in all PEgRNA pairs examined. Specifically efficient editing with 5′ PEgRNA having a spacer identified by SEQ ID No. 77, 536, or 1019 (5′ spacer No. 5, 8, or 18) paired with 3′ PEgRNA having a spacer identified by SEQ ID No. 1900, 1936, or 2673 (3′ spacer No. 8, 10, or 17) was observed consistently in all three patient derived cell lines.

TABLE 56
Dual prime editing for CTG excision in patient iPSCs.

| | 5′ | | | 3′ | | | | |
|---|---|---|---|---|---|---|---|---|
| 5′ | pegRNA | | 3′ | pegRNA | | | | |
| spacer | SEQ ID | 5′ PBS | spacer | SEQ ID | 3′ PBS | % editing | %editing | % editing |
| No. | NO: | length | No. | NO: | length | efficiency_iPSC1 | efficiency_iPSC2 | efficiency_iPSC3 |
| 5 | 94 | 10 | 5 | 1542 | 10 | + | ++ | + |
| 5 | 94 | 10 | 8 | 1916 | 10 | ++ | ++ | ++ |
| 5 | 94 | 10 | 10 | 1952 | 10 | +++ | +++ | ++ |
| 5 | 94 | 10 | 11 | 2279 | 10 | + | + | + |
| 5 | 94 | 10 | 12 | 2297 | 10 | + | + | + |
| 5 | 94 | 10 | 13 | 2315 | 10 | ++ | + | + |
| 5 | 94 | 10 | 16 | 2369 | 10 | + | + | + |
| 5 | 94 | 10 | 17 | 2689 | 10 | ++ | ++ | ++ |
| 6 | 501 | 10 | 5 | 1542 | 10 | + | + | + |
| 6 | 501 | 10 | 8 | 1916 | 10 | ++ | ++ | + |
| 6 | 501 | 10 | 10 | 1952 | 10 | +++ | +++ | ++ |
| 6 | 501 | 10 | 11 | 2279 | 10 | + | + | + |
| 6 | 501 | 10 | 12 | 2297 | 10 | + | + | + |
| 6 | 501 | 10 | 13 | 2315 | 10 | + | + | + |
| 6 | 501 | 10 | 16 | 2369 | 10 | + | + | + |
| 6 | 501 | 10 | 17 | 2689 | 10 | NA | ++ | + |
| 8 | 552 | 10 | 5 | 1542 | 10 | + | + | + |
| 8 | 552 | 10 | 8 | 1916 | 10 | ++ | ++ | ++ |
| 8 | 552 | 10 | 10 | 1952 | 10 | +++ | +++ | ++ |
| 8 | 552 | 10 | 11 | 2279 | 10 | + | + | + |
| 8 | 552 | 10 | 12 | 2297 | 10 | + | + | + |
| 8 | 552 | 10 | 13 | 2315 | 10 | + | + | + |
| 8 | 552 | 10 | 16 | 2369 | 10 | + | + | + |
| 8 | 552 | 10 | 17 | 2689 | 10 | ++ | ++ | + |
| 11 | 606 | 10 | 5 | 1542 | 10 | + | + | + |
| 11 | 606 | 10 | 8 | 1916 | 10 | + | + | + |
| 11 | 606 | 10 | 10 | 1952 | 10 | + | + | + |
| 11 | 606 | 10 | 11 | 2279 | 10 | + | + | + |
| 11 | 606 | 10 | 12 | 2297 | 10 | + | + | + |
| 11 | 606 | 10 | 13 | 2315 | 10 | + | + | + |
| 11 | 606 | 10 | 16 | 2369 | 10 | + | + | + |
| 11 | 606 | 10 | 17 | 2689 | 10 | + | + | + |
| 18 | 1035 | 10 | 5 | 1542 | 10 | + | + | + |
| 18 | 1035 | 10 | 8 | 1916 | 10 | ++ | ++ | ++ |
| 18 | 1035 | 10 | 10 | 1952 | 10 | +++ | +++ | ++ |
| 18 | 1035 | 10 | 11 | 2279 | 10 | + | + | + |
| 18 | 1035 | 10 | 12 | 2297 | 10 | + | + | + |
| 18 | 1035 | 10 | 13 | 2315 | 10 | ++ | + | + |
| 18 | 1035 | 10 | 16 | 2369 | 10 | + | + | + |
| 18 | 1035 | 10 | 17 | 2689 | 10 | ++ | ++ | ++ |
| 19 | 1357 | 10 | 5 | 1542 | 10 | + | + | + |
| 19 | 1357 | 10 | 8 | 1916 | 10 | + | + | + |
| 19 | 1357 | 10 | 10 | 1952 | 10 | ++ | ++ | + |
| 19 | 1357 | 10 | 11 | 2279 | 10 | + | + | + |
| 19 | 1357 | 10 | 12 | 2297 | 10 | + | + | + |
| 19 | 1357 | 10 | 13 | 2315 | 10 | + | + | + |
| 19 | 1357 | 10 | 16 | 2369 | 10 | + | + | + |
| 19 | 1357 | 10 | 17 | 2689 | 10 | + | + | + |

To further validate dual prime editing in DM1 patient derived cells, three 5′ pegRNAs and three 3′ pegRNAs were examined for editing efficiency in DM1 patient cells iPSC1. Five different doses of plasmid encoding the pegRNA was used (very low, low, mid, high, and very high) ranging from 0.1 ng to 20 ng per reaction. The iPSC cells were transfected, gRNA extracted and sequenced, and prime editing efficiency assessed as described above in this Example. The results are summarized in Table 57.

In Table 57, each row presents information for a pair of PEgRNAs (i.e., a 5′ PEgRNA and a 3′ PEgRNA) used in dual prime editing. Each PEgRNA is identified by a sequence identifier (“SEQ ID NO”). Two negative control conditions were included: 1) non-transfected iPSC1 cells (“NTC control”) and 2) iPSC1 cells transfected with nucleic acid expressing a green fluorescence protein (“GFP control”). The following scale was used for editing efficiency: PEgRNA pairs exhibiting editing efficiency of 0% and up to 2.5% are indicated with “−”, PEgRNA pairs exhibiting editing efficiency above 2.5% and up to 5% are indicated with “+”, PEgRNA pairs exhibiting editing efficiency above 5% and up to 15% are indicated with “++”, PEgRNA pairs exhibiting editing efficiency above 15% and up to 35% are indicated with “+++”, and PEgRNA pairs exhibiting editing efficiency above 35% and up to 65% are indicated with “++++”. Efficient dual prime editing was observed for each PEgRNA pair at mid or above-mid PEgRNA doses. For each PEgRNA pair, the editing efficiency showed dose dependency on the amount of PEgRNA used, indicating bona fide editing of the DMPK gene.

TABLE 57
Dual prime editing for CTG excision in patient iPSCs.

| 5′ PEgRNA | 3′ PEgRNA | PEgRNA | % Editing |
|---|---|---|---|
| SEQ ID NO: | SEQ ID NO: | Dose | Efficiency |
| 94 | 1916 | very high | ++++ |
| 94 | 1916 | very high | +++ |
| 94 | 1916 | high | ++++ |
| 94 | 1916 | high | +++ |
| 94 | 1916 | mid | +++ |
| 94 | 1916 | mid | ++ |
| 94 | 1916 | low | + |
| 94 | 1916 | low | + |
| 94 | 1916 | very low | − |
| 94 | 1916 | very low | ++ |
| 94 | 1952 | very high | ++++ |
| 94 | 1952 | very high | ++++ |
| 94 | 1952 | high | +++ |
| 94 | 1952 | high | ++++ |
| 94 | 1952 | mid | ++ |
| 94 | 1952 | mid | ++ |
| 94 | 1952 | low | + |
| 94 | 1952 | low | ++ |
| 94 | 1952 | very low | + |
| 94 | 1952 | very low | + |
| 94 | 2689 | very high | +++ |
| 94 | 2689 | very high | ++++ |
| 94 | 2689 | high | +++ |
| 94 | 2689 | high | +++ |
| 94 | 2689 | mid | ++ |
| 94 | 2689 | mid | ++ |
| 94 | 2689 | low | + |
| 94 | 2689 | low | + |
| 94 | 2689 | very low | + |
| 94 | 2689 | very low | + |
| 552 | 1916 | very high | ++++ |
| 552 | 1916 | very high | ++++ |
| 552 | 1916 | high | ++++ |
| 552 | 1916 | high | ++++ |
| 552 | 1916 | mid | +++ |
| 552 | 1916 | mid | +++ |
| 552 | 1916 | low | ++ |
| 552 | 1916 | low | ++ |
| 552 | 1916 | very low | + |
| 552 | 1916 | very low | + |
| 552 | 1952 | very high | ++++ |
| 552 | 1952 | very high | ++++ |
| 552 | 1952 | high | ++++ |
| 552 | 1952 | high | ++++ |
| 552 | 1952 | mid | +++ |
| 552 | 1952 | mid | +++ |
| 552 | 1952 | low | ++ |
| 552 | 1952 | low | ++ |
| 552 | 1952 | very low | ++ |
| 552 | 1952 | very low | + |
| 552 | 2689 | very high | +++ |
| 552 | 2689 | very high | ++++ |
| 552 | 2689 | high | +++ |
| 552 | 2689 | high | +++ |
| 552 | 2689 | mid | ++ |
| 552 | 2689 | mid | ++ |
| 552 | 2689 | low | + |
| 552 | 2689 | low | + |
| 552 | 2689 | very low | − |
| 552 | 2689 | very low | + |
| 1035 | 1916 | very high | +++ |
| 1035 | 1916 | very high | ++++ |
| 1035 | 1916 | high | ++++ |
| 1035 | 1916 | high | ++++ |
| 1035 | 1916 | mid | +++ |
| 1035 | 1916 | mid | +++ |
| 1035 | 1916 | low | + |
| 1035 | 1916 | low | ++ |
| 1035 | 1916 | very low | − |
| 1035 | 1916 | very low | − |
| 1035 | 1952 | very high | ++++ |
| 1035 | 1952 | very high | ++++ |
| 1035 | 1952 | high | ++++ |
| 1035 | 1952 | high | ++++ |
| 1035 | 1952 | mid | ++++ |
| 1035 | 1952 | mid | +++ |
| 1035 | 1952 | low | ++ |
| 1035 | 1952 | low | +++ |
| 1035 | 1952 | very low | − |
| 1035 | 1952 | very low | − |
| 1035 | 2689 | very high | +++ |
| 1035 | 2689 | very high | ++++ |
| 1035 | 2689 | high | +++ |
| 1035 | 2689 | high | +++ |
| 1035 | 2689 | mid | +++ |
| 1035 | 2689 | mid | +++ |
| 1035 | 2689 | low | − |
| 1035 | 2689 | low | + |
| 1035 | 2689 | very low | − |
| 1035 | 2689 | very low | − |
| GFP control | GFP control | NA | − |
| GFP control | GFP control | NA | − |
| GFP control | GFP control | NA | − |
| NTC control | NTC control | NA | − |
| NTC control | NTC control | NA | − |
| NTC control | NTC control | NA | − |

### Example 6: Dual Prime Editing for Excision of CTG Repeats in DM1 Patient iPSC Cells

5′ PEgRNAs having spacer No. 5 paired with 3′ PEgRNAs having spacer No. 10 were further examined. Different PBS lengths were tested in DM1 patient cells iPSC2. The cells were transfected, gRNA harvested and sequenced, and CTG repeat excision and attB integration assessed as described in Example 5 above. The results are summarized in Table 58. In Table 58, each row presents information for a pair of PEgRNAs (i.e., a 5′ PEgRNA and a 3′ PEgRNA) used in dual prime editing. Each PEgRNA is identified by a sequence identifier (“SEQ ID NO”). The following scale was used for editing efficiency: PEgRNA pairs exhibiting editing efficiency above 1% and up to 5% are indicated with “+”, PEgRNA pairs exhibiting editing efficiency above 5% and up to 1000 are indicated with “++”, PEgRNA pairs exhibiting editing efficiency above 1000 and up to 2000 are indicated with “+++”, and PEgRNA pairs exhibiting editing efficiency above 2000 and up to 4000 are indicated with “++++”. Successful dual prime editing was observed for all PEgRNA pairs tested. In addition, particularly efficient prime editing was observed with PEgRNAs having PBS lengths of 8, 9, 10, 11, or 12 nucleotides.

TABLE 58
Dual prime editing for CTG
excision in patient iPSCs.

| 5′ pegRNA | 5′ pegRNA | 3′ pegRNA | 3′ pegRNA | % editing |
|---|---|---|---|---|
| SEQ ID NO: | PBS length | SEQ ID NO: | PBS length | efficiency |
| 93 | 8 | 1954 | 8 | ++++ |
| 93 | 8 | 1955 | 9 | ++++ |
| 93 | 8 | 1952 | 10 | ++++ |
| 93 | 8 | 1956 | 11 | +++ |
| 93 | 8 | 1953 | 12 | ++++ |
| 93 | 8 | 1957 | 13 | ++ |
| 93 | 8 | 1958 | 14 | ++ |
| 93 | 8 | 1959 | 15 | +++ |
| 93 | 8 | 1960 | 16 | + |
| 93 | 8 | 1961 | 17 | + |
| 94 | 10 | 1954 | 8 | ++++ |
| 94 | 10 | 1955 | 9 | ++++ |
| 94 | 10 | 1952 | 10 | ++++ |
| 94 | 10 | 1956 | 11 | ++ |
| 94 | 10 | 1953 | 12 | ++++ |
| 94 | 10 | 1957 | 13 | ++ |
| 94 | 10 | 1958 | 14 | +++ |
| 94 | 10 | 1959 | 15 | +++ |
| 94 | 10 | 1960 | 16 | + |
| 94 | 10 | 1961 | 17 | + |
| 95 | 13 | 1954 | 8 | ++++ |
| 95 | 13 | 1955 | 9 | +++ |
| 95 | 13 | 1952 | 10 | ++++ |
| 95 | 13 | 1956 | 11 | +++ |
| 95 | 13 | 1953 | 12 | ++++ |
| 95 | 13 | 1957 | 13 | ++ |
| 95 | 13 | 1958 | 14 | +++ |
| 95 | 13 | 1959 | 15 | +++ |
| 95 | 13 | 1960 | 16 | + |
| 95 | 13 | 1961 | 17 | + |
| 96 | 9 | 1954 | 8 | ++++ |
| 96 | 9 | 1955 | 9 | +++ |
| 96 | 9 | 1952 | 10 | +++ |
| 96 | 9 | 1956 | 11 | +++ |
| 96 | 9 | 1953 | 12 | ++++ |
| 96 | 9 | 1957 | 13 | ++ |
| 96 | 9 | 1958 | 14 | ++ |
| 96 | 9 | 1959 | 15 | +++ |
| 96 | 9 | 1960 | 16 | + |
| 96 | 9 | 1961 | 17 | + |
| 97 | 10 | 1954 | 8 | ++++ |
| 97 | 10 | 1955 | 9 | ++++ |
| 97 | 10 | 1952 | 10 | ++++ |
| 97 | 10 | 1956 | 11 | +++ |
| 97 | 10 | 1953 | 12 | ++++ |
| 97 | 10 | 1957 | 13 | ++ |
| 97 | 10 | 1958 | 14 | +++ |
| 97 | 10 | 1959 | 15 | +++ |
| 97 | 10 | 1960 | 16 | + |
| 97 | 10 | 1961 | 17 | + |
| 98 | 12 | 1954 | 8 | +++ |
| 98 | 12 | 1955 | 9 | +++ |
| 98 | 12 | 1952 | 10 | + |
| 98 | 12 | 1956 | 11 | + |
| 98 | 12 | 1953 | 12 | ++ |
| 98 | 12 | 1957 | 13 | + |
| 98 | 12 | 1958 | 14 | +++ |
| 98 | 12 | 1959 | 15 | + |
| 98 | 12 | 1960 | 16 | + |
| 98 | 12 | 1961 | 17 | + |
| 99 | 14 | 1954 | 8 | ++++ |
| 99 | 14 | 1955 | 9 | +++ |
| 99 | 14 | 1952 | 10 | +++ |
| 99 | 14 | 1956 | 11 | +++ |
| 99 | 14 | 1953 | 12 | +++ |
| 99 | 14 | 1957 | 13 | ++ |
| 99 | 14 | 1958 | 14 | ++ |
| 99 | 14 | 1959 | 15 | +++ |
| 99 | 14 | 1960 | 16 | + |
| 99 | 14 | 1961 | 17 | + |
| 100 | 15 | 1954 | 8 | ++++ |
| 100 | 15 | 1955 | 9 | ++++ |
| 100 | 15 | 1952 | 10 | ++++ |
| 100 | 15 | 1956 | 11 | +++ |
| 100 | 15 | 1953 | 12 | ++++ |
| 100 | 15 | 1957 | 13 | ++ |
| 100 | 15 | 1958 | 14 | ++ |
| 100 | 15 | 1959 | 15 | +++ |
| 100 | 15 | 1960 | 16 | + |
| 100 | 15 | 1961 | 17 | + |
| 101 | 16 | 1954 | 8 | + |
| 101 | 16 | 1955 | 9 | ++ |
| 101 | 16 | 1952 | 10 | + |
| 101 | 16 | 1956 | 11 | + |
| 101 | 16 | 1953 | 12 | ++ |
| 101 | 16 | 1957 | 13 | + |
| 101 | 16 | 1958 | 14 | + |
| 101 | 16 | 1959 | 15 | + |
| 101 | 16 | 1960 | 16 | + |
| 101 | 16 | 1961 | 17 | + |
| 102 | 17 | 1954 | 8 | +++ |
| 102 | 17 | 1955 | 9 | +++ |
| 102 | 17 | 1952 | 10 | +++ |
| 102 | 17 | 1956 | 11 | ++ |
| 102 | 17 | 1953 | 12 | +++ |
| 102 | 17 | 1957 | 13 | ++ |
| 102 | 17 | 1958 | 14 | ++ |
| 102 | 17 | 1959 | 15 | ++ |
| 102 | 17 | 1960 | 16 | + |
| 102 | 17 | 1961 | 17 | + |

### Example 7. Dual Prime Editing Assay for Additional RTT Sequences

For PEgRNAs that replace the CTG repeats with a complementarity region between the 3′ RTT and the 5′ RTT, different PEgRNA pairs were tested to examine the effect of complementarity region length and GC % on dual prime editing.

A HEK293T cell line was generated to stably express a transgene cassette driven by a promoter, and harbors from 5′ to 3′ a GFP open reading frame (ORF), a 1 kb random sequence, and a mcherry ORF. The 1 kb sequence contains multiple stop codons. Prior to editing, only the GFP is expressed. DMPK PEgRNA protospacer and PAM sequence were cloned at the ends flanking of the 1 kb random sequence. Successful prime editing with the DMPK pegRNAs would replace the 1 kb random sequence with the OD encoded by the PEgRNA RTT sequences, which would permit simultaneous expression of GFP and mcherry. FACS was used as the readout of editing efficiency, which is calculated as: (number of GFP+ and mcherry+ cells)/(number of GFP+ cells).

The results are summarized in Table 59. In Table 59, each row presents information for a pair of PEgRNAs (i.e., a 5′ PEgRNA and a 3′ PEgRNA) used in dual prime editing. Each PEgRNA is identified by a sequence identifier (“SEQ ID NO”). 5′ PEgRNAs having spacer No. 5 paired with 3′ PEgRNAs having spacer No. 3 were tested. All PEgRNAs had a PBS length of 12 nt. The RTT length in this experiment reflects the length of the region of complementarity between a 5′ RTT and a 3′RTT. Different lengths GC % of the region of complementarity were tested. The following scale was used for editing efficiency: PEgRNA pairs exhibiting editing efficiency above 0.9% and up to 5% are indicated with “+”, PEgRNA pairs exhibiting editing efficiency above 5% and up to 10% are indicated with “++”, PEgRNA pairs exhibiting editing efficiency above 10% and up to 15% are indicated with “+++”, and PEgRNA pairs exhibiting editing efficiency above 15% and up to 20% are indicated with “++++”.

TABLE 59
Dual prime editing pegRNAs in reporter cells

| 5′ PERNA | 3′ pegRNA | PEgRNA | | | |
|---|---|---|---|---|---|
| SEQ | SEQ | RTT | RTT | RTT | % editing |
| ID NO: | ID NO: | paring No. | length | GC % | efficiency |
| 3495 | 3496 | 1 | 38 | 0.63 | +++ |
| 103 | 1425 | 2 | 38 | 0.421052632 | ++ |
| 104 | 1426 | 3 | 38 | 0.421052632 | + |
| 105 | 1427 | 4 | 38 | 0.421052632 | + |
| 106 | 1428 | 5 | 38 | 0.421052632 | + |
| 107 | 1429 | 6 | 38 | 0.421052632 | ++ |
| 108 | 1430 | 7 | 38 | 0.421052632 | + |
| 109 | 1431 | 8 | 38 | 0.421052632 | + |
| 110 | 1432 | 9 | 38 | 0.421052632 | + |
| 111 | 1433 | 10 | 38 | 0.421052632 | ++ |
| 112 | 1434 | 11 | 38 | 0.526315789 | ++ |
| 113 | 1435 | 12 | 38 | 0.526315789 | ++ |
| 114 | 1436 | 13 | 38 | 0.526315789 | + |
| 115 | 1437 | 14 | 38 | 0.526315789 | ++ |
| 116 | 1438 | 15 | 38 | 0.526315789 | ++ |
| 117 | 1439 | 16 | 38 | 0.526315789 | ++ |
| 118 | 1440 | 17 | 38 | 0.526315789 | ++ |
| 119 | 1441 | 18 | 38 | 0.526315789 | ++ |
| 120 | 1442 | 19 | 38 | 0.526315789 | + |
| 121 | 1443 | 20 | 38 | 0.631578947 | ++ |
| 122 | 1444 | 21 | 38 | 0.631578947 | ++ |
| 123 | 1445 | 22 | 38 | 0.631578947 | + |
| 124 | 1446 | 23 | 38 | 0.631578947 | ++ |
| 125 | 1447 | 24 | 38 | 0.631578947 | +++ |
| 126 | 1448 | 25 | 38 | 0.631578947 | ++++ |
| 127 | 1449 | 26 | 38 | 0.631578947 | ++ |
| 128 | 1450 | 27 | 38 | 0.631578947 | +++ |
| 129 | 1451 | 28 | 38 | 0.631578947 | ++ |
| 130 | 1452 | 29 | 38 | 0.710526316 | ++ |
| 131 | 1453 | 30 | 38 | 0.710526316 | + |
| 132 | 1454 | 31 | 38 | 0.710526316 | ++ |
| 133 | 1455 | 32 | 38 | 0.710526316 | ++ |
| 134 | 1456 | 33 | 38 | 0.710526316 | ++++ |
| 135 | 1457 | 34 | 38 | 0.710526316 | +++ |
| 136 | 1458 | 35 | 38 | 0.710526316 | +++ |
| 137 | 1459 | 36 | 38 | 0.710526316 | ++ |
| 138 | 1460 | 37 | 38 | 0.710526316 | +++ |
| 139 | 1461 | 38 | 38 | 0.789473684 | ++ |
| 140 | 1462 | 39 | 38 | 0.789473684 | ++ |
| 141 | 1463 | 40 | 38 | 0.789473684 | ++ |
| 142 | 1464 | 41 | 38 | 0.789473684 | ++ |
| 143 | 1465 | 42 | 38 | 0.789473684 | +++ |
| 144 | 1466 | 43 | 38 | 0.789473684 | +++ |
| 145 | 1467 | 44 | 38 | 0.789473684 | ++ |
| 146 | 1468 | 45 | 38 | 0.789473684 | + |
| 147 | 1469 | 46 | 38 | 0.789473684 | +++ |
| 148 | 1470 | 47 | 23 | 0.652173913 | + |
| 149 | 1471 | 48 | 23 | 0.652173913 | +++ |
| 150 | 1472 | 49 | 23 | 0.652173913 | ++ |
| 151 | 1473 | 50 | 23 | 0.652173913 | ++ |
| 152 | 1474 | 51 | 23 | 0.652173913 | +++ |
| 153 | 1475 | 52 | 23 | 0.652173913 | +++ |
| 154 | 1476 | 53 | 23 | 0.652173913 | ++ |
| 155 | 1477 | 54 | 23 | 0.652173913 | ++++ |
| 156 | 1478 | 55 | 23 | 0.652173913 | + |
| 157 | 1479 | 56 | 53 | 0.622641509 | ++ |
| 158 | 1480 | 57 | 53 | 0.622641509 | ++ |
| 159 | 1481 | 58 | 53 | 0.622641509 | + |
| 160 | 1482 | 59 | 53 | 0.622641509 | ++ |
| 161 | 1483 | 60 | 68 | 0.632352941 | + |
| 162 | 1484 | 61 | 68 | 0.632352941 | + |
| 163 | 1485 | 62 | 68 | 0.632352941 | ++ |
| 164 | 1486 | 63 | 68 | 0.632352941 | + |
| 165 | 1487 | 64 | 68 | 0.632352941 | + |
| 166 | 1488 | 65 | 68 | 0.632352941 | ++ |
| 167 | 1489 | 66 | 68 | 0.632352941 | +++ |
| 168 | 1490 | 67 | 68 | 0.632352941 | + |
| 169 | 1491 | 68 | 68 | 0.632352941 | + |
| 170 | 1492 | 69 | 83 | 0.626506024 | + |
| 171 | 1493 | 70 | 83 | 0.626506024 | + |
| 172 | 1494 | 71 | 83 | 0.626506024 | ++ |
| 173 | 1495 | 72 | 83 | 0.626506024 | + |
| 174 | 1496 | 73 | 83 | 0.626506024 | + |
| 175 | 1497 | 74 | 83 | 0.626506024 | + |
| 176 | 1498 | 75 | 83 | 0.626506024 | ++ |
| 177 | 1499 | 76 | 83 | 0.626506024 | ++ |
| 178 | 1500 | 77 | 83 | 0.626506024 | ++ |
| 179 | 1501 | 78 | 83 | 0.626506024 | + |
| 180 | 1502 | 79 | 24 | 0.5 | + |

As shown in Table 59, successful dual prime editing was observed in all PEgRNA pairs tested. Specifically, the RTTs of each PEgRNA pair in rows 2-47 of Table 59 (RTT Paring No.s 1-46) have 38 base pairs of region of complementarity to each other, and the regions of complementarity have varying GC % as shown in Table 59. The RTTs of each PEgRNA pair in rows 48-79 of Table 59 (RTT Paring No.s 47-48) have a region of complementarity to each other, wherein the regions of complementarity have GC % of about 63%-65%, and have variant lengths as shown in Table 59. The median editing efficiency of fixed-length, varying GC % and fixed-GC %, varying length complementarity regions are further summarized in Table 60 below. Increased editing efficiency was observed for PEgRNA pairs that have regions of complementarity between RTTs that have 63%, 71%, or 79% of GC content (nearly double the median efficiency of PEgRNA pairs having RTT complementarity region having 42% GC), or PEgRNA pairs that have regions of complementarity between RTTs that are 23, 38, or 53 base pairs in length.

TABLE 60
Summary of median editing values of PEgRNA pairs.

| RTT length | RTT GC % | Median Editing efficiency |
|---|---|---|
| 38 | 42% | + |
| | 53% | ++ |
| | 63% | ++ |
| | 71% | ++ |
| | 79% | ++ |
| 23 | 63-65% | ++ |
| 53 | | ++ |
| 68 | | + |
| 83 | | + |

### Example 8. Dual Prime Editing pegRNAs in Single Prime Editing System

To assess the PEgRNAs' characteristics as independent guide RNAs for prime editing, 5′ and 3′ PEgRNAs designed for dual prime editing were tested for editing efficiency in a single prime editing system.

Single prime editing was performed using a lentiviral pooled screen assay, as described in Kim et al, 2021 (pubmed.ncbi.nlm.nih.gov/32958957/). Briefly, a lentiviral plasmid library was designed, with each library member harboring a sequence encoding a U6 promoter-driven PEgRNA and a target sequence corresponding to the protospacer, PAM and an editing target sequence having complementarity to the PEgRNA RTT. The library was transduced at low MOI (0.1-0.3) in HEK293T cells, resulting in random genomic integration of one copy of a target sequence and a pegRNA sequence in the vast majority of cells. Following puromycin selection for successful transduction, cells were transfected with polynucleotide encoding a prime editor fusion protein and incubated for 3 days. Following incubation, genomic DNA was extracted and analyzed by deep amplicon sequencing of the target site. Editing efficiency was evaluated by alignment rates to the edited and reference amplicons using CRISPresso2. Indel rates were aggregated from the reference and edited amplicons using a 17 bp window around the nick site.

PEgRNAs having 5′ spacer numbers 5, 18, and 11 and 3′ spacer numbers 5, 10, and 16, each having a designed RTT sequence having a specific length and GC % were designed. Each PEgRNA has a PBS of 10 nucleotides in length. The varying lengths and GC % of the RTT of each PEgRNA are shown in Tables 61-66. The single prime editing results are scaled as follows and summarized in Tables 61-66.

For each of Tables 61-66, the following scale was used for editing efficiency: PEgRNAs exhibiting editing efficiency of 0% are indicated with “−”, PEgRNAs exhibiting editing efficiency above 0% and up to 5% are indicated with “+”, PEgRNAs exhibiting editing efficiency above 5% and up to 10% are indicated with “++”, PEgRNAs exhibiting editing efficiency above 10% and up to 15% are indicated with “+++”, and PEgRNAs exhibiting editing efficiency above 15% and up to 35% are indicated with “++++”. The following scale was used for indel rates: PEgRNAs exhibiting indel rates of 0% are indicated with “−”, PEgRNAs exhibiting indel rates above 0% and up to 1% are indicated with “+”, PEgRNAs exhibiting indel rates above 1% and up to 10% are indicated with “++”, PEgRNAs exhibiting indel rates above 10% and up to 20% are indicated with “+++”, and PEgRNAs exhibiting indel rates above 20% and up to 75% are indicated with “++++”. The results show the PEgRNAs' ability to mediate prime editing in a single prime editing system, which indicate successful binding of the spacer to the search target sequence, and successful interaction among the PEgRNA components and with the Cas9 protein.

TABLE 61
dual prime editing pegRNAs tested with single prime editing system

| | pegRNA | RTT | | | | |
|---|---|---|---|---|---|---|
| 5′ | SEQ ID | paring | RTT | RTT | % editing | % indel |
| spacer | NO: | No. | length | GC % | efficiency | frequency |
| 5 | 181 | 260 | 25 | 0.48 | + | + |
| 5 | 182 | 92 | 30 | 0.5 | − | ++++ |
| 5 | 183 | 146 | 30 | 0.6 | − | − |
| 5 | 185 | 235 | 20 | 0.5 | ++ | ++ |
| 5 | 188 | 239 | 15 | 0.27 | ++ | ++ |
| 5 | 189 | 113 | 18 | 0.39 | ++ | ++ |
| 5 | 190 | 133 | 15 | 0.67 | +++ | + |
| 5 | 192 | 102 | 15 | 0.53 | ++ | + |
| 5 | 194 | 288 | 20 | 0.6 | ++ | + |
| 5 | 196 | 364 | 15 | 0.67 | + | + |
| 5 | 198 | 353 | 20 | 0.4 | ++ | ++ |
| 5 | 199 | 249 | 18 | 0.61 | + | ++ |
| 5 | 200 | 363 | 22 | 0.59 | + | − |
| 5 | 201 | 257 | 18 | 0.39 | + | ++ |
| 5 | 203 | 234 | 20 | 0.5 | ++++ | + |
| 5 | 204 | 135 | 20 | 0.85 | − | − |
| 5 | 205 | 192 | 28 | 0.5 | + | ++ |
| 5 | 206 | 282 | 18 | 0.72 | − | − |
| 5 | 208 | 270 | 20 | 0.6 | + | + |
| 5 | 209 | 318 | 10 | 0.8 | + | + |
| 5 | 211 | 350 | 22 | 0.41 | +++ | − |
| 5 | 212 | 132 | 15 | 0.4 | +++ | ++ |
| 5 | 213 | 315 | 22 | 0.5 | + | + |
| 5 | 214 | 370 | 22 | 0.41 | +++ | + |
| 5 | 215 | 366 | 25 | 0.4 | ++ | + |
| 5 | 217 | 221 | 28 | 0.29 | ++++ | − |
| 5 | 221 | 263 | 18 | 0.78 | − | ++++ |
| 5 | 222 | 369 | 25 | 0.64 | + | − |
| 5 | 223 | 180 | 25 | 0.48 | ++ | + |
| 5 | 226 | 287 | 15 | 0.67 | + | ++ |
| 5 | 227 | 170 | 15 | 0.53 | ++ | − |
| 5 | 228 | 323 | 30 | 0.5 | − | − |
| 5 | 229 | 381 | 40 | 0.4 | ++ | ++ |
| 5 | 231 | 149 | 10 | 0.9 | − | − |
| 5 | 232 | 104 | 30 | 0.4 | + | + |
| 5 | 235 | 103 | 10 | 0.5 | + | − |
| 5 | 236 | 215 | 18 | 0.72 | + | + |
| 5 | 238 | 138 | 18 | 0.39 | + | ++ |
| 5 | 239 | 356 | 28 | 0.5 | + | + |
| 5 | 240 | 305 | 22 | 0.59 | ++ | + |
| 5 | 241 | 266 | 20 | 0.8 | − | + |
| 5 | 242 | 142 | 22 | 0.82 | − | − |
| 5 | 243 | 218 | 15 | 0.93 | − | + |
| 5 | 248 | 303 | 18 | 0.83 | − | − |
| 5 | 249 | 211 | 15 | 0.87 | − | − |
| 5 | 252 | 209 | 18 | 0.78 | + | − |
| 5 | 255 | 373 | 20 | 0.75 | − | ++ |
| 5 | 256 | 157 | 10 | 0.8 | + | ++ |
| 5 | 259 | 151 | 15 | 0.93 | − | − |
| 5 | 263 | 322 | 40 | 0.6 | − | ++ |
| 5 | 266 | 172 | 20 | 0.65 | − | ++ |
| 5 | 268 | 205 | 10 | 0.8 | + | + |
| 5 | 269 | 124 | 28 | 0.61 | + | ++ |
| 5 | 272 | 219 | 18 | 0.61 | + | − |
| 5 | 273 | 213 | 15 | 0.93 | + | + |
| 5 | 280 | 383 | 28 | 0.5 | + | + |
| 5 | 281 | 293 | 18 | 0.89 | − | − |
| 5 | 283 | 188 | 22 | 0.68 | − | +++ |
| 5 | 285 | 179 | 15 | 0.73 | + | ++ |
| 5 | 286 | 181 | 10 | 0.8 | − | + |
| 5 | 290 | 375 | 18 | 0.67 | + | − |
| 5 | 294 | 155 | 20 | 0.75 | + | − |
| 5 | 295 | 128 | 20 | 0.6 | + | − |
| 5 | 296 | 175 | 25 | 0.64 | − | − |
| 5 | 297 | 243 | 18 | 0.67 | + | + |
| 5 | 299 | 307 | 20 | 0.65 | + | − |
| 5 | 300 | 329 | 15 | 0.67 | + | + |
| 5 | 301 | 286 | 30 | 0.5 | + | ++ |
| 5 | 302 | 145 | 10 | 0.6 | + | + |
| 5 | 304 | 290 | 20 | 0.7 | + | ++ |
| 5 | 305 | 321 | 10 | 0.8 | + | ++ |
| 5 | 306 | 186 | 20 | 0.3 | + | − |
| 5 | 308 | 283 | 18 | 0.67 | ++ | ++ |
| 5 | 309 | 384 | 10 | 0.5 | + | + |
| 5 | 310 | 223 | 15 | 0.8 | + | ++ |
| 5 | 312 | 361 | 15 | 0.6 | + | − |
| 5 | 313 | 216 | 25 | 0.6 | − | + |
| 5 | 314 | 278 | 20 | 0.5 | + | ++ |
| 5 | 315 | 246 | 15 | 0.6 | + | ++ |
| 5 | 316 | 291 | 28 | 0.64 | − | − |
| 5 | 318 | 229 | 10 | 0.6 | + | + |
| 5 | 319 | 184 | 15 | 0.73 | + | − |
| 5 | 320 | 169 | 10 | 0.9 | + | + |
| 5 | 322 | 161 | 20 | 0.85 | + | ++ |
| 5 | 325 | 379 | 10 | 0.6 | + | ++ |
| 5 | 327 | 137 | 15 | 0.67 | + | − |
| 5 | 328 | 231 | 22 | 0.64 | − | ++++ |
| 5 | 330 | 190 | 15 | 0.87 | − | + |
| 5 | 332 | 166 | 18 | 0.78 | + | ++ |
| 5 | 333 | 141 | 10 | 0.7 | + | − |
| 5 | 335 | 348 | 20 | 0.8 | − | − |
| 5 | 344 | 140 | 18 | 0.89 | − | − |
| 5 | 346 | 376 | 22 | 0.5 | ++ | ++ |
| 5 | 347 | 269 | 15 | 0.87 | − | − |
| 5 | 349 | 131 | 22 | 0.59 | + | + |
| 5 | 350 | 312 | 18 | 0.83 | + | ++ |
| 5 | 353 | 280 | 15 | 0.6 | + | ++ |
| 5 | 354 | 125 | 22 | 0.64 | − | − |
| 5 | 355 | 334 | 18 | 0.78 | − | ++ |
| 5 | 356 | 232 | 28 | 0.61 | − | − |
| 5 | 358 | 355 | 35 | 0.4 | + | ++ |
| 5 | 360 | 325 | 15 | 0.4 | + | + |
| 5 | 362 | 139 | 25 | 0.72 | − | − |
| 5 | 364 | 114 | 18 | 0.78 | + | ++ |
| 5 | 369 | 108 | 10 | 0.7 | + | + |
| 5 | 373 | 349 | 23 | 0.65 | ++ | + |
| 5 | 374 | 330 | 30 | 0.4 | ++ | − |
| 5 | 375 | 119 | 30 | 0.4 | ++++ | − |
| 5 | 376 | 252 | 35 | 0.51 | + | + |
| 5 | 379 | 241 | 10 | 0.6 | + | ++ |
| 5 | 380 | 338 | 20 | 0.4 | ++++ | + |
| 5 | 381 | 115 | 15 | 0.67 | + | ++ |
| 5 | 387 | 107 | 15 | 0.4 | ++ | ++ |
| 5 | 389 | 127 | 22 | 0.5 | ++ | ++ |
| 5 | 390 | 253 | 30 | 0.3 | ++ | ++ |
| 5 | 391 | 80 | 18 | 0.28 | +++ | ++ |
| 5 | 392 | 197 | 18 | 0.83 | − | + |
| 5 | 393 | 371 | 40 | 0.3 | ++ | − |
| 5 | 394 | 111 | 35 | 0.4 | + | ++ |
| 5 | 395 | 228 | 28 | 0.39 | ++ | + |
| 5 | 396 | 367 | 22 | 0.32 | ++ | ++ |
| 5 | 397 | 251 | 35 | 0.29 | ++ | − |
| 5 | 398 | 292 | 30 | 0.7 | − | − |
| 5 | 401 | 225 | 22 | 0.64 | + | + |
| 5 | 403 | 159 | 20 | 0.65 | + | − |
| 5 | 404 | 116 | 18 | 0.72 | − | − |
| 5 | 406 | 279 | 15 | 0.53 | + | − |
| 5 | 407 | 337 | 28 | 0.39 | ++++ | ++ |
| 5 | 408 | 91 | 18 | 0.61 | +++ | ++ |
| 5 | 409 | 83 | 25 | 0.4 | ++++ | + |
| 5 | 410 | 240 | 25 | 0.32 | ++ | − |
| 5 | 411 | 345 | 15 | 0.8 | + | ++ |
| 5 | 412 | 220 | 25 | 0.6 | ++ | + |
| 5 | 413 | 106 | 15 | 0.8 | + | ++ |
| 5 | 414 | 182 | 20 | 0.7 | − | − |
| 5 | 415 | 183 | 15 | 0.73 | − | ++ |
| 5 | 416 | 331 | 25 | 0.48 | + | − |
| 5 | 419 | 242 | 18 | 0.5 | ++ | ++ |
| 5 | 420 | 150 | 20 | 0.7 | + | + |
| 5 | 421 | 158 | 25 | 0.6 | − | + |
| 5 | 422 | 174 | 10 | 0.8 | − | + |
| 5 | 423 | 327 | 20 | 0.8 | − | − |
| 5 | 424 | 99 | 35 | 0.51 | − | +++ |
| 5 | 426 | 185 | 25 | 0.4 | + | + |
| 5 | 427 | 227 | 28 | 0.39 | + | + |
| 5 | 429 | 341 | 18 | 0.5 | + | − |
| 5 | 430 | 339 | 35 | 0.4 | ++ | ++ |
| 5 | 432 | 324 | 18 | 0.5 | ++ | ++ |
| 5 | 433 | 342 | 22 | 0.41 | ++++ | + |
| 5 | 434 | 87 | 20 | 0.4 | +++ | + |
| 11 | 609 | 119 | 30 | 0.4 | + | + |
| 11 | 611 | 124 | 28 | 0.61 | − | − |
| 11 | 612 | 125 | 22 | 0.64 | − | − |
| 11 | 613 | 127 | 22 | 0.5 | + | − |
| 11 | 615 | 128 | 20 | 0.6 | + | − |
| 11 | 616 | 83 | 25 | 0.4 | + | − |
| 11 | 617 | 131 | 22 | 0.59 | − | − |
| 11 | 618 | 132 | 15 | 0.4 | − | ++ |
| 11 | 619 | 133 | 15 | 0.67 | − | − |
| 11 | 623 | 137 | 15 | 0.67 | − | ++ |
| 11 | 624 | 138 | 18 | 0.39 | − | − |
| 11 | 625 | 139 | 25 | 0.72 | − | − |
| 11 | 627 | 141 | 10 | 0.7 | + | − |
| 11 | 629 | 143 | 22 | 0.86 | − | − |
| 11 | 630 | 145 | 10 | 0.6 | − | ++ |
| 11 | 635 | 150 | 20 | 0.7 | + | ++ |
| 11 | 636 | 151 | 15 | 0.93 | − | − |
| 11 | 640 | 155 | 20 | 0.75 | − | − |
| 11 | 643 | 158 | 25 | 0.6 | − | + |
| 11 | 644 | 159 | 20 | 0.65 | + | − |
| 11 | 646 | 161 | 20 | 0.85 | − | ++ |
| 11 | 654 | 169 | 10 | 0.9 | − | − |
| 11 | 655 | 170 | 15 | 0.53 | − | − |
| 11 | 657 | 172 | 20 | 0.65 | − | − |
| 11 | 659 | 174 | 10 | 0.8 | − | ++ |
| 11 | 664 | 87 | 20 | 0.4 | − | − |
| 11 | 665 | 179 | 15 | 0.73 | − | − |
| 11 | 666 | 180 | 25 | 0.48 | + | ++ |
| 11 | 667 | 181 | 10 | 0.8 | − | − |
| 11 | 668 | 182 | 20 | 0.7 | − | − |
| 11 | 669 | 183 | 15 | 0.73 | − | + |
| 11 | 670 | 184 | 15 | 0.73 | − | ++ |
| 11 | 671 | 185 | 25 | 0.4 | − | − |
| 11 | 672 | 186 | 20 | 0.3 | + | − |
| 11 | 673 | 188 | 22 | 0.68 | − | − |
| 11 | 675 | 190 | 15 | 0.87 | − | − |
| 11 | 677 | 192 | 28 | 0.5 | − | − |
| 11 | 682 | 198 | 20 | 0.9 | − | − |
| 11 | 688 | 205 | 10 | 0.8 | − | − |
| 11 | 692 | 209 | 18 | 0.78 | − | ++ |
| 11 | 695 | 92 | 30 | 0.5 | − | + |
| 11 | 699 | 215 | 18 | 0.72 | + | − |
| 11 | 700 | 216 | 25 | 0.6 | − | + |
| 11 | 703 | 219 | 18 | 0.61 | − | − |
| 11 | 704 | 220 | 25 | 0.6 | + | + |
| 11 | 705 | 221 | 28 | 0.29 | + | ++ |
| 11 | 707 | 223 | 15 | 0.8 | + | − |
| 11 | 708 | 225 | 22 | 0.64 | + | − |
| 11 | 710 | 227 | 28 | 0.39 | + | ++ |
| 11 | 711 | 228 | 28 | 0.39 | + | ++ |
| 11 | 712 | 229 | 10 | 0.6 | − | − |
| 11 | 715 | 232 | 28 | 0.61 | + | ++ |
| 11 | 717 | 234 | 20 | 0.5 | + | − |
| 11 | 718 | 235 | 20 | 0.5 | + | + |
| 11 | 722 | 239 | 15 | 0.27 | − | − |
| 11 | 723 | 240 | 25 | 0.32 | + | − |
| 11 | 724 | 241 | 10 | 0.6 | − | − |
| 11 | 725 | 242 | 18 | 0.5 | − | − |
| 11 | 726 | 243 | 18 | 0.67 | − | − |
| 11 | 729 | 246 | 15 | 0.6 | − | − |
| 11 | 730 | 249 | 18 | 0.61 | − | − |
| 11 | 731 | 251 | 35 | 0.29 | + | − |
| 11 | 733 | 253 | 30 | 0.3 | + | + |
| 11 | 737 | 257 | 18 | 0.39 | − | ++ |
| 11 | 739 | 260 | 25 | 0.48 | + | − |
| 11 | 746 | 266 | 20 | 0.8 | − | − |
| 11 | 750 | 270 | 20 | 0.6 | − | − |
| 11 | 758 | 278 | 20 | 0.5 | + | ++ |
| 11 | 759 | 279 | 15 | 0.53 | − | ++ |
| 11 | 760 | 280 | 15 | 0.6 | − | − |
| 11 | 763 | 283 | 18 | 0.67 | − | − |
| 11 | 766 | 286 | 30 | 0.5 | − | ++ |
| 11 | 768 | 287 | 15 | 0.67 | − | − |
| 11 | 769 | 288 | 20 | 0.6 | + | ++ |
| 11 | 771 | 290 | 20 | 0.7 | + | ++ |
| 11 | 773 | 292 | 30 | 0.7 | − | − |
| 11 | 785 | 305 | 22 | 0.59 | + | − |
| 11 | 786 | 102 | 15 | 0.53 | − | − |
| 11 | 787 | 307 | 20 | 0.65 | + | + |
| 11 | 793 | 315 | 22 | 0.5 | + | +++ |
| 11 | 795 | 318 | 10 | 0.8 | − | − |
| 11 | 796 | 321 | 10 | 0.8 | − | − |
| 11 | 797 | 322 | 40 | 0.6 | − | − |
| 11 | 801 | 327 | 20 | 0.8 | − | − |
| 11 | 803 | 330 | 30 | 0.4 | + | − |
| 11 | 804 | 331 | 25 | 0.48 | + | ++ |
| 11 | 806 | 103 | 10 | 0.5 | − | − |
| 11 | 807 | 80 | 18 | 0.28 | − | ++ |
| 11 | 809 | 334 | 18 | 0.78 | − | ++++ |
| 11 | 812 | 337 | 28 | 0.39 | − | − |
| 11 | 813 | 338 | 20 | 0.4 | + | ++ |
| 11 | 814 | 339 | 35 | 0.4 | + | − |
| 11 | 815 | 104 | 30 | 0.4 | − | ++ |
| 11 | 816 | 341 | 18 | 0.5 | + | ++ |
| 11 | 817 | 342 | 22 | 0.41 | + | − |
| 11 | 823 | 348 | 20 | 0.8 | − | ++ |
| 11 | 824 | 349 | 23 | 0.65 | − | − |
| 11 | 826 | 350 | 22 | 0.41 | + | − |
| 11 | 828 | 353 | 20 | 0.4 | − | ++ |
| 11 | 829 | 355 | 35 | 0.4 | + | − |
| 11 | 830 | 356 | 28 | 0.5 | − | + |
| 11 | 837 | 363 | 22 | 0.59 | + | ++ |
| 11 | 838 | 107 | 15 | 0.4 | + | − |
| 11 | 841 | 366 | 25 | 0.4 | + | ++ |
| 11 | 842 | 367 | 22 | 0.32 | + | ++ |
| 11 | 843 | 108 | 10 | 0.7 | − | ++ |
| 11 | 844 | 369 | 25 | 0.64 | + | ++ |
| 11 | 845 | 370 | 22 | 0.41 | + | ++ |
| 11 | 846 | 371 | 40 | 0.3 | + | − |
| 11 | 848 | 373 | 20 | 0.75 | − | − |
| 11 | 849 | 374 | 28 | 0.61 | − | ++ |
| 11 | 851 | 376 | 22 | 0.5 | + | − |
| 11 | 854 | 381 | 40 | 0.4 | + | − |
| 11 | 855 | 383 | 28 | 0.5 | − | − |
| 11 | 856 | 384 | 10 | 0.5 | − | − |
| 11 | 857 | 111 | 35 | 0.4 | + | ++ |
| 11 | 860 | 115 | 15 | 0.67 | − | − |
| 18 | 1038 | 119 | 30 | 0.4 | + | + |
| 18 | 1040 | 124 | 28 | 0.61 | − | − |
| 18 | 1041 | 125 | 22 | 0.64 | − | − |
| 18 | 1042 | 127 | 22 | 0.5 | +++ | − |
| 18 | 1045 | 83 | 25 | 0.4 | +++ | + |
| 18 | 1046 | 131 | 22 | 0.59 | − | − |
| 18 | 1047 | 132 | 15 | 0.4 | + | + |
| 18 | 1048 | 133 | 15 | 0.67 | − | + |
| 18 | 1049 | 134 | 28 | 0.64 | − | − |
| 18 | 1052 | 137 | 15 | 0.67 | + | + |
| 18 | 1053 | 138 | 18 | 0.39 | ++ | − |
| 18 | 1056 | 141 | 10 | 0.7 | + | + |
| 18 | 1057 | 142 | 22 | 0.82 | − | − |
| 18 | 1059 | 145 | 10 | 0.6 | ++ | − |
| 18 | 1060 | 146 | 30 | 0.6 | − | − |
| 18 | 1063 | 149 | 10 | 0.9 | + | − |
| 18 | 1064 | 150 | 20 | 0.7 | − | − |
| 18 | 1065 | 151 | 15 | 0.93 | − | + |
| 18 | 1068 | 154 | 22 | 0.73 | − | − |
| 18 | 1069 | 155 | 20 | 0.75 | − | − |
| 18 | 1071 | 157 | 10 | 0.8 | + | − |
| 18 | 1072 | 158 | 25 | 0.6 | − | − |
| 18 | 1073 | 159 | 20 | 0.65 | ++++ | − |
| 18 | 1075 | 161 | 20 | 0.85 | − | ++ |
| 18 | 1080 | 166 | 18 | 0.78 | − | − |
| 18 | 1083 | 169 | 10 | 0.9 | + | + |
| 18 | 1084 | 170 | 15 | 0.53 | + | − |
| 18 | 1086 | 172 | 20 | 0.65 | − | − |
| 18 | 1088 | 174 | 10 | 0.8 | + | − |
| 18 | 1089 | 175 | 25 | 0.64 | − | − |
| 18 | 1093 | 87 | 20 | 0.4 | ++ | − |
| 18 | 1094 | 179 | 15 | 0.73 | − | − |
| 18 | 1095 | 180 | 25 | 0.48 | + | ++ |
| 18 | 1096 | 181 | 10 | 0.8 | − | + |
| 18 | 1097 | 182 | 20 | 0.7 | − | ++ |
| 18 | 1098 | 183 | 15 | 0.73 | − | − |
| 18 | 1099 | 184 | 15 | 0.73 | + | + |
| 18 | 1100 | 185 | 25 | 0.4 | + | ++ |
| 18 | 1101 | 186 | 20 | 0.3 | ++ | − |
| 18 | 1102 | 188 | 22 | 0.68 | − | − |
| 18 | 1104 | 190 | 15 | 0.87 | − | − |
| 18 | 1106 | 192 | 28 | 0.5 | − | ++++ |
| 18 | 1110 | 197 | 18 | 0.83 | − | − |
| 18 | 1117 | 205 | 10 | 0.8 | + | +++ |
| 18 | 1119 | 91 | 18 | 0.61 | + | ++ |
| 18 | 1121 | 209 | 18 | 0.78 | − | +++ |
| 18 | 1123 | 211 | 15 | 0.87 | + | − |
| 18 | 1126 | 213 | 15 | 0.93 | + | + |
| 18 | 1128 | 215 | 18 | 0.72 | − | ++ |
| 18 | 1131 | 218 | 15 | 0.93 | − | − |
| 18 | 1132 | 219 | 18 | 0.61 | + | − |
| 18 | 1133 | 220 | 25 | 0.6 | + | ++ |
| 18 | 1134 | 221 | 28 | 0.29 | ++ | + |
| 18 | 1136 | 223 | 15 | 0.8 | + | + |
| 18 | 1137 | 225 | 22 | 0.64 | − | − |
| 18 | 1139 | 227 | 28 | 0.39 | + | − |
| 18 | 1140 | 228 | 28 | 0.39 | ++ | + |
| 18 | 1141 | 229 | 10 | 0.6 | ++ | − |
| 18 | 1144 | 232 | 28 | 0.61 | + | − |
| 18 | 1146 | 234 | 20 | 0.5 | +++ | + |
| 18 | 1147 | 235 | 20 | 0.5 | + | + |
| 18 | 1151 | 239 | 15 | 0.27 | ++ | − |
| 18 | 1152 | 240 | 25 | 0.32 | +++ | − |
| 18 | 1153 | 241 | 10 | 0.6 | + | − |
| 18 | 1154 | 242 | 18 | 0.5 | + | ++ |
| 18 | 1155 | 243 | 18 | 0.67 | + | + |
| 18 | 1156 | 244 | 30 | 0.73 | + | − |
| 18 | 1158 | 246 | 15 | 0.6 | + | − |
| 18 | 1159 | 249 | 18 | 0.61 | − | − |
| 18 | 1160 | 251 | 35 | 0.29 | ++ | ++ |
| 18 | 1162 | 253 | 30 | 0.3 | +++ | ++ |
| 18 | 1166 | 257 | 18 | 0.39 | + | + |
| 18 | 1168 | 260 | 25 | 0.48 | + | + |
| 18 | 1175 | 266 | 20 | 0.8 | − | − |
| 18 | 1178 | 269 | 15 | 0.87 | − | − |
| 18 | 1179 | 270 | 20 | 0.6 | − | +++ |
| 18 | 1187 | 278 | 20 | 0.5 | + | + |
| 18 | 1188 | 279 | 15 | 0.53 | + | − |
| 18 | 1189 | 280 | 15 | 0.6 | + | − |
| 18 | 1192 | 283 | 18 | 0.67 | + | + |
| 18 | 1195 | 286 | 30 | 0.5 | − | − |
| 18 | 1197 | 287 | 15 | 0.67 | + | ++ |
| 18 | 1198 | 288 | 20 | 0.6 | ++ | − |
| 18 | 1200 | 290 | 20 | 0.7 | + | ++ |
| 18 | 1202 | 292 | 30 | 0.7 | − | − |
| 18 | 1203 | 293 | 18 | 0.89 | − | − |
| 18 | 1214 | 305 | 22 | 0.59 | + | ++ |
| 18 | 1215 | 102 | 15 | 0.53 | + | − |
| 18 | 1216 | 307 | 20 | 0.65 | + | − |
| 18 | 1220 | 312 | 18 | 0.83 | − | ++ |
| 18 | 1222 | 315 | 22 | 0.5 | ++ | ++ |
| 18 | 1224 | 318 | 10 | 0.8 | + | ++ |
| 18 | 1225 | 321 | 10 | 0.8 | + | − |
| 18 | 1228 | 324 | 18 | 0.5 | + | + |
| 18 | 1229 | 325 | 15 | 0.4 | ++ | ++ |
| 18 | 1231 | 329 | 15 | 0.67 | + | ++ |
| 18 | 1232 | 330 | 30 | 0.4 | + | − |
| 18 | 1233 | 331 | 25 | 0.48 | + | + |
| 18 | 1235 | 103 | 10 | 0.5 | + | − |
| 18 | 1236 | 80 | 18 | 0.28 | ++ | − |
| 18 | 1238 | 334 | 18 | 0.78 | − | ++ |
| 18 | 1241 | 337 | 28 | 0.39 | + | + |
| 18 | 1242 | 338 | 20 | 0.4 | + | ++ |
| 18 | 1243 | 339 | 35 | 0.4 | + | ++ |
| 18 | 1244 | 104 | 30 | 0.4 | − | − |
| 18 | 1245 | 341 | 18 | 0.5 | + | − |
| 18 | 1246 | 342 | 22 | 0.41 | ++ | ++ |
| 18 | 1249 | 345 | 15 | 0.8 | − | − |
| 18 | 1253 | 349 | 23 | 0.65 | + | + |
| 18 | 1254 | 106 | 15 | 0.8 | − | − |
| 18 | 1255 | 350 | 22 | 0.41 | ++ | − |
| 18 | 1257 | 353 | 20 | 0.4 | + | ++ |
| 18 | 1258 | 355 | 35 | 0.4 | − | ++ |
| 18 | 1259 | 356 | 28 | 0.5 | + | − |
| 18 | 1262 | 359 | 22 | 0.68 | − | ++ |
| 18 | 1264 | 361 | 15 | 0.6 | + | + |
| 18 | 1266 | 363 | 22 | 0.59 | − | − |
| 18 | 1267 | 107 | 15 | 0.4 | ++ | − |
| 18 | 1268 | 364 | 15 | 0.67 | + | − |
| 18 | 1270 | 366 | 25 | 0.4 | + | ++ |
| 18 | 1271 | 367 | 22 | 0.32 | + | + |
| 18 | 1272 | 108 | 10 | 0.7 | + | ++ |
| 18 | 1273 | 369 | 25 | 0.64 | + | ++ |
| 18 | 1274 | 370 | 22 | 0.41 | +++ | +++ |
| 18 | 1275 | 371 | 40 | 0.3 | + | − |
| 18 | 1277 | 373 | 20 | 0.75 | − | + |
| 18 | 1279 | 375 | 18 | 0.67 | + | ++ |
| 18 | 1280 | 376 | 22 | 0.5 | ++ | + |
| 18 | 1282 | 379 | 10 | 0.6 | + | − |
| 18 | 1283 | 381 | 40 | 0.4 | + | − |
| 18 | 1284 | 383 | 28 | 0.5 | + | ++ |
| 18 | 1285 | 384 | 10 | 0.5 | + | − |
| 18 | 1286 | 111 | 35 | 0.4 | + | − |
| 18 | 1287 | 113 | 18 | 0.39 | ++ | − |
| 18 | 1288 | 114 | 18 | 0.78 | − | − |
| 18 | 1289 | 115 | 15 | 0.67 | ++ | − |

TABLE 62
dual prime editing pegRNAs tested with single prime editing system

| | pegRNA | RTT | | | | |
|---|---|---|---|---|---|---|
| 5′ | SEQ ID | pairing | RTT | RTT | % editing | % indel |
| spacer | NO: | No. | length | GC % | efficiency | frequency |
| 5 | 436 | 306 | 20 | 0.7 | + | + |
| 5 | 436 | 306 | 20 | 0.7 | + | ++ |
| 5 | 438 | 112 | 15 | 0.67 | ++ | ++ |
| 5 | 438 | 112 | 15 | 0.67 | + | ++ |
| 5 | 439 | 109 | 20 | 0.4 | ++++ | ++ |
| 5 | 440 | 320 | 15 | 0.67 | + | − |
| 5 | 441 | 368 | 20 | 0.5 | ++ | − |
| 5 | 442 | 382 | 20 | 0.4 | + | ++ |
| 5 | 442 | 382 | 20 | 0.4 | ++ | ++ |
| 5 | 446 | 85 | 15 | 0.8 | − | +++ |
| 5 | 446 | 85 | 15 | 0.8 | − | − |
| 5 | 447 | 351 | 25 | 0.48 | + | + |
| 5 | 448 | 121 | 20 | 0.5 | +++ | + |
| 5 | 449 | 86 | 25 | 0.48 | + | + |
| 5 | 449 | 86 | 25 | 0.48 | + | ++ |
| 5 | 450 | 90 | 20 | 0.8 | + | ++ |
| 5 | 450 | 90 | 20 | 0.8 | + | ++ |
| 5 | 450 | 90 | 20 | 0.8 | + | − |
| 5 | 451 | 300 | 25 | 0.6 | + | + |
| 5 | 454 | 378 | 15 | 0.8 | − | − |
| 5 | 456 | 123 | 20 | 0.6 | ++ | ++ |
| 5 | 456 | 123 | 20 | 0.6 | ++ | + |
| 5 | 456 | 123 | 20 | 0.6 | + | ++ |
| 5 | 457 | 110 | 20 | 0.7 | − | − |
| 5 | 459 | 380 | 20 | 0.5 | ++ | ++ |
| 5 | 460 | 89 | 25 | 0.4 | + | ++ |
| 5 | 460 | 89 | 25 | 0.4 | + | − |
| 5 | 461 | 247 | 25 | 0.4 | + | − |
| 5 | 462 | 88 | 30 | 0.4 | +++ | + |
| 5 | 462 | 88 | 30 | 0.4 | +++ | − |
| 5 | 462 | 88 | 30 | 0.4 | ++ | + |
| 5 | 463 | 354 | 15 | 0.4 | +++ | + |
| 5 | 464 | 130 | 30 | 0.5 | + | − |
| 5 | 464 | 130 | 30 | 0.5 | + | ++ |
| 5 | 464 | 130 | 30 | 0.5 | + | − |
| 5 | 465 | 81 | 15 | 0.4 | ++++ | − |
| 5 | 466 | 122 | 15 | 0.4 | ++++ | + |
| 5 | 467 | 129 | 15 | 0.53 | +++ | + |
| 5 | 467 | 129 | 15 | 0.53 | ++ | + |
| 5 | 467 | 129 | 15 | 0.53 | ++ | + |
| 5 | 468 | 96 | 15 | 0.6 | ++ | ++ |
| 5 | 468 | 96 | 15 | 0.6 | ++ | + |
| 5 | 468 | 96 | 15 | 0.6 | +++ | + |
| 11 | 863 | 84 | 25 | 0.6 | − | − |
| 11 | 865 | 86 | 25 | 0.48 | − | ++ |
| 11 | 866 | 89 | 25 | 0.4 | − | ++ |
| 11 | 867 | 90 | 20 | 0.8 | − | + |
| 11 | 870 | 96 | 15 | 0.6 | − | − |
| 11 | 870 | 96 | 15 | 0.6 | + | ++ |
| 11 | 872 | 109 | 20 | 0.4 | + | − |
| 11 | 873 | 112 | 15 | 0.67 | − | − |
| 11 | 873 | 112 | 15 | 0.67 | + | − |
| 11 | 874 | 122 | 15 | 0.4 | + | ++ |
| 11 | 875 | 126 | 15 | 0.4 | − | +++ |
| 11 | 876 | 129 | 15 | 0.53 | − | ++ |
| 11 | 876 | 129 | 15 | 0.53 | − | − |
| 11 | 876 | 129 | 15 | 0.53 | − | − |
| 11 | 878 | 144 | 30 | 0.4 | + | + |
| 11 | 880 | 206 | 30 | 0.4 | + | + |
| 11 | 880 | 206 | 30 | 0.4 | + | + |
| 11 | 882 | 247 | 25 | 0.4 | − | ++ |
| 11 | 883 | 250 | 15 | 0.6 | − | − |
| 11 | 884 | 272 | 25 | 0.6 | − | − |
| 11 | 886 | 306 | 20 | 0.7 | − | − |
| 11 | 886 | 306 | 20 | 0.7 | + | ++ |
| 11 | 886 | 306 | 20 | 0.7 | − | − |
| 11 | 889 | 319 | 20 | 0.6 | − | − |
| 11 | 889 | 319 | 20 | 0.6 | − | − |
| 11 | 889 | 319 | 20 | 0.6 | − | ++ |
| 11 | 890 | 320 | 15 | 0.67 | − | − |
| 11 | 892 | 340 | 25 | 0.4 | + | ++ |
| 11 | 893 | 351 | 25 | 0.48 | + | − |
| 11 | 893 | 351 | 25 | 0.48 | + | − |
| 11 | 894 | 354 | 15 | 0.4 | − | − |
| 11 | 895 | 368 | 20 | 0.5 | + | − |
| 11 | 895 | 368 | 20 | 0.5 | + | + |
| 11 | 895 | 368 | 20 | 0.5 | − | − |
| 11 | 896 | 382 | 20 | 0.4 | + | + |
| 11 | 896 | 382 | 20 | 0.4 | + | − |
| 18 | 1292 | 85 | 15 | 0.8 | + | − |
| 18 | 1293 | 86 | 25 | 0.48 | − | − |
| 18 | 1294 | 88 | 30 | 0.4 | + | + |
| 18 | 1295 | 89 | 25 | 0.4 | − | ++ |
| 18 | 1295 | 89 | 25 | 0.4 | + | − |
| 18 | 1296 | 90 | 20 | 0.8 | − | ++ |
| 18 | 1296 | 90 | 20 | 0.8 | − | − |
| 18 | 1299 | 96 | 15 | 0.6 | + | − |
| 18 | 1299 | 96 | 15 | 0.6 | + | − |
| 18 | 1299 | 96 | 15 | 0.6 | + | ++ |
| 18 | 1300 | 105 | 15 | 0.8 | − | − |
| 18 | 1301 | 109 | 20 | 0.4 | +++ | ++ |
| 18 | 1303 | 112 | 15 | 0.67 | + | − |
| 18 | 1303 | 112 | 15 | 0.67 | + | ++ |
| 18 | 1304 | 122 | 15 | 0.4 | ++ | ++ |
| 18 | 1304 | 122 | 15 | 0.4 | +++ | ++ |
| 18 | 1305 | 123 | 20 | 0.6 | − | ++ |
| 18 | 1306 | 126 | 15 | 0.4 | ++ | + |
| 18 | 1307 | 129 | 15 | 0.53 | +++ | ++ |
| 18 | 1307 | 129 | 15 | 0.53 | ++ | ++ |
| 18 | 1307 | 129 | 15 | 0.53 | ++ | ++ |
| 18 | 1308 | 130 | 30 | 0.5 | − | − |
| 18 | 1308 | 130 | 30 | 0.5 | − | ++ |
| 18 | 1308 | 130 | 30 | 0.5 | − | − |
| 18 | 1311 | 247 | 25 | 0.4 | + | ++ |
| 18 | 1313 | 259 | 30 | 0.4 | − | − |
| 18 | 1315 | 300 | 25 | 0.6 | − | ++ |
| 18 | 1316 | 306 | 20 | 0.7 | + | − |
| 18 | 1320 | 320 | 15 | 0.67 | + | − |
| 18 | 1322 | 351 | 25 | 0.48 | + | ++ |
| 18 | 1323 | 378 | 15 | 0.8 | − | − |
| 18 | 1324 | 380 | 20 | 0.5 | + | − |
| 18 | 1324 | 380 | 20 | 0.5 | + | − |
| 18 | 1324 | 380 | 20 | 0.5 | + | − |
| 18 | 1325 | 382 | 20 | 0.4 | +++ | − |
| 18 | 1325 | 382 | 20 | 0.4 | + | ++ |

TABLE 63
dual prime editing pegRNAs tested with single prime editing system

| | pegRNA | RTT | | | | |
|---|---|---|---|---|---|---|
| 5′ | SEQ ID | pairing | RTT | | % editing | % indel |
| spacer | NO: | No. | length | RTT GC % | efficiency | frequency |
| 5 | 469 | 387 | 12 | 0.666666667 | + | + |
| 5 | 470 | 387 | 12 | 0.666666667 | + | + |
| 5 | 471 | 388 | 12 | 0.666666667 | + | ++ |
| 5 | 472 | 388 | 12 | 0.666666667 | + | ++ |
| 5 | 473 | 386 | 12 | 0.583333333 | + | + |
| 5 | 474 | 388 | 12 | 0.666666667 | + | ++ |
| 5 | 475 | 386 | 12 | 0.583333333 | − | + |
| 5 | 476 | 386 | 12 | 0.583333333 | − | − |
| 5 | 477 | 388 | 12 | 0.666666667 | ++ | ++ |
| 5 | 478 | 388 | 12 | 0.666666667 | + | + |
| 5 | 479 | 386 | 12 | 0.583333333 | + | + |
| 5 | 480 | 386 | 12 | 0.583333333 | + | ++ |
| 5 | 481 | 387 | 12 | 0.666666667 | + | − |
| 5 | 482 | 387 | 12 | 0.666666667 | + | ++ |
| 5 | 483 | 387 | 12 | 0.666666667 | + | + |
| 11 | 897 | 388 | 12 | 0.666666667 | + | ++ |
| 11 | 898 | 388 | 12 | 0.666666667 | + | ++ |
| 11 | 899 | 388 | 12 | 0.666666667 | − | − |
| 11 | 900 | 388 | 12 | 0.666666667 | − | − |
| 11 | 902 | 386 | 12 | 0.583333333 | − | ++ |
| 11 | 903 | 386 | 12 | 0.583333333 | − | ++ |
| 11 | 904 | 386 | 12 | 0.583333333 | + | − |
| 11 | 905 | 386 | 12 | 0.583333333 | + | ++ |
| 11 | 906 | 386 | 12 | 0.583333333 | − | + |
| 11 | 907 | 387 | 12 | 0.666666667 | − | + |
| 11 | 908 | 387 | 12 | 0.666666667 | + | − |
| 11 | 909 | 387 | 12 | 0.666666667 | + | + |
| 11 | 910 | 387 | 12 | 0.666666667 | + | + |
| 11 | 911 | 387 | 12 | 0.666666667 | − | ++ |
| 18 | 1326 | 388 | 12 | 0.666666667 | + | ++ |
| 18 | 1327 | 388 | 12 | 0.666666667 | ++ | ++ |
| 18 | 1328 | 388 | 12 | 0.666666667 | + | + |
| 18 | 1329 | 388 | 12 | 0.666666667 | + | − |
| 18 | 1331 | 386 | 12 | 0.583333333 | + | − |
| 18 | 1332 | 386 | 12 | 0.583333333 | + | ++ |
| 18 | 1333 | 386 | 12 | 0.583333333 | + | − |
| 18 | 1334 | 386 | 12 | 0.583333333 | + | − |
| 18 | 1335 | 386 | 12 | 0.583333333 | + | ++ |
| 18 | 1336 | 387 | 12 | 0.666666667 | + | − |
| 18 | 1337 | 387 | 12 | 0.666666667 | + | + |
| 18 | 1338 | 387 | 12 | 0.666666667 | ++ | + |
| 18 | 1339 | 387 | 12 | 0.666666667 | + | + |
| 18 | 1340 | 387 | 12 | 0.666666667 | + | − |

TABLE 64
dual prime editing pegRNAs tested with single
prime editing system

| | pegRNA | RTT | | | | |
|---|---|---|---|---|---|---|
| 3′ | SEQ ID | paring | RTT | RTT | % editing | % indel |
| spacer | NO: | No. | length | GC % | efficiency | frequency |
| 5 | 1563 | 366 | 25 | 0.4 | + | + |
| 5 | 1565 | 221 | 28 | 0.29 | + | − |
| 5 | 1566 | 235 | 20 | 0.5 | + | − |
| 5 | 1567 | 374 | 28 | 0.61 | − | − |
| 5 | 1570 | 263 | 18 | 0.78 | − | − |
| 5 | 1572 | 180 | 25 | 0.48 | + | + |
| 5 | 1575 | 287 | 15 | 0.67 | + | − |
| 5 | 1576 | 170 | 15 | 0.53 | + | − |
| 5 | 1577 | 323 | 30 | 0.5 | + | + |
| 5 | 1578 | 381 | 40 | 0.4 | − | + |
| 5 | 1580 | 149 | 10 | 0.9 | − | − |
| 5 | 1581 | 104 | 30 | 0.4 | + | ++ |
| 5 | 1584 | 103 | 10 | 0.5 | ++ | − |
| 5 | 1586 | 134 | 28 | 0.64 | − | − |
| 5 | 1587 | 138 | 18 | 0.39 | + | − |
| 5 | 1588 | 356 | 28 | 0.5 | + | ++ |
| 5 | 1589 | 305 | 22 | 0.59 | +++ | + |
| 5 | 1590 | 266 | 20 | 0.8 | − | − |
| 5 | 1591 | 142 | 22 | 0.82 | − | + |
| 5 | 1592 | 218 | 15 | 0.93 | − | − |
| 5 | 1598 | 303 | 18 | 0.83 | + | − |
| 5 | 1599 | 211 | 15 | 0.87 | − | ++ |
| 5 | 1605 | 239 | 15 | 0.27 | + | + |
| 5 | 1607 | 373 | 20 | 0.75 | + | + |
| 5 | 1608 | 157 | 10 | 0.8 | ++ | − |
| 5 | 1609 | 113 | 18 | 0.39 | ++ | +++ |
| 5 | 1618 | 133 | 15 | 0.67 | + | + |
| 5 | 1620 | 172 | 20 | 0.65 | − | + |
| 5 | 1622 | 205 | 10 | 0.8 | + | ++ |
| 5 | 1623 | 124 | 28 | 0.61 | − | − |
| 5 | 1627 | 213 | 15 | 0.93 | − | − |
| 5 | 1634 | 383 | 28 | 0.5 | + | − |
| 5 | 1635 | 293 | 18 | 0.89 | − | − |
| 5 | 1637 | 188 | 22 | 0.68 | − | − |
| 5 | 1639 | 179 | 15 | 0.73 | + | − |
| 5 | 1640 | 181 | 10 | 0.8 | + | − |
| 5 | 1644 | 375 | 18 | 0.67 | − | ++ |
| 5 | 1645 | 260 | 25 | 0.48 | ++ | ++ |
| 5 | 1651 | 128 | 20 | 0.6 | + | + |
| 5 | 1652 | 175 | 25 | 0.64 | − | − |
| 5 | 1653 | 243 | 18 | 0.67 | + | − |
| 5 | 1655 | 307 | 20 | 0.65 | ++ | ++ |
| 5 | 1656 | 102 | 15 | 0.53 | + | ++ |
| 5 | 1657 | 329 | 15 | 0.67 | − | ++ |
| 5 | 1658 | 286 | 30 | 0.5 | + | − |
| 5 | 1659 | 145 | 10 | 0.6 | + | ++ |
| 5 | 1660 | 244 | 30 | 0.73 | − | ++ |
| 5 | 1661 | 290 | 20 | 0.7 | +++ | − |
| 5 | 1662 | 321 | 10 | 0.8 | + | ++ |
| 5 | 1663 | 186 | 20 | 0.3 | + | ++ |
| 5 | 1666 | 283 | 18 | 0.67 | − | ++ |
| 5 | 1667 | 384 | 10 | 0.5 | − | ++ |
| 5 | 1670 | 288 | 20 | 0.6 | + | − |
| 5 | 1671 | 361 | 15 | 0.6 | + | − |
| 5 | 1674 | 278 | 20 | 0.5 | + | − |
| 5 | 1675 | 246 | 15 | 0.6 | − | ++ |
| 5 | 1676 | 291 | 28 | 0.64 | − | ++ |
| 5 | 1678 | 364 | 15 | 0.67 | + | − |
| 5 | 1679 | 229 | 10 | 0.6 | + | ++ |
| 5 | 1680 | 184 | 15 | 0.73 | + | − |
| 5 | 1681 | 169 | 10 | 0.9 | − | ++ |
| 5 | 1683 | 161 | 20 | 0.85 | − | + |
| 5 | 1686 | 379 | 10 | 0.6 | + | ++ |
| 5 | 1688 | 137 | 15 | 0.67 | + | + |
| 5 | 1690 | 231 | 22 | 0.64 | + | − |
| 5 | 1692 | 190 | 15 | 0.87 | − | ++ |
| 5 | 1694 | 353 | 20 | 0.4 | + | ++ |
| 5 | 1696 | 141 | 10 | 0.7 | + | ++ |
| 5 | 1698 | 348 | 20 | 0.8 | − | − |
| 5 | 1706 | 249 | 18 | 0.61 | + | − |
| 5 | 1709 | 140 | 18 | 0.89 | − | + |
| 5 | 1711 | 376 | 22 | 0.5 | + | − |
| 5 | 1712 | 269 | 15 | 0.87 | + | − |
| 5 | 1714 | 257 | 18 | 0.39 | + | − |
| 5 | 1715 | 131 | 22 | 0.59 | + | − |
| 5 | 1719 | 280 | 15 | 0.6 | − | + |
| 5 | 1720 | 125 | 22 | 0.64 | + | + |
| 5 | 1721 | 334 | 18 | 0.78 | − | − |
| 5 | 1722 | 232 | 28 | 0.61 | + | ++ |
| 5 | 1724 | 92 | 30 | 0.5 | + | − |
| 5 | 1726 | 355 | 35 | 0.4 | + | − |
| 5 | 1728 | 325 | 15 | 0.4 | + | ++++ |
| 5 | 1730 | 139 | 25 | 0.72 | − | ++ |
| 5 | 1732 | 114 | 18 | 0.78 | − | ++ |
| 5 | 1733 | 234 | 20 | 0.5 | ++ | − |
| 5 | 1738 | 135 | 20 | 0.85 | − | − |
| 5 | 1739 | 108 | 10 | 0.7 | + | − |
| 5 | 1744 | 330 | 30 | 0.4 | + | − |
| 5 | 1745 | 119 | 30 | 0.4 | + | − |
| 5 | 1746 | 252 | 35 | 0.51 | − | − |
| 5 | 1749 | 241 | 10 | 0.6 | + | − |
| 5 | 1750 | 338 | 20 | 0.4 | + | − |
| 5 | 1751 | 115 | 15 | 0.67 | − | + |
| 5 | 1757 | 107 | 15 | 0.4 | + | ++ |
| 5 | 1758 | 154 | 22 | 0.73 | + | − |
| 5 | 1759 | 127 | 22 | 0.5 | + | − |
| 5 | 1760 | 253 | 30 | 0.3 | ++ | − |
| 5 | 1761 | 80 | 18 | 0.28 | + | + |
| 5 | 1762 | 197 | 18 | 0.83 | − | ++ |
| 5 | 1763 | 371 | 40 | 0.3 | + | ++ |
| 5 | 1764 | 111 | 35 | 0.4 | − | + |
| 5 | 1765 | 228 | 28 | 0.39 | + | ++ |
| 5 | 1766 | 192 | 28 | 0.5 | + | − |
| 5 | 1767 | 367 | 22 | 0.32 | + | − |
| 5 | 1768 | 251 | 35 | 0.29 | + | + |
| 5 | 1769 | 292 | 30 | 0.7 | − | − |
| 5 | 1774 | 159 | 20 | 0.65 | + | − |
| 5 | 1775 | 282 | 18 | 0.72 | + | ++ |
| 5 | 1776 | 116 | 18 | 0.72 | − | − |
| 5 | 1778 | 279 | 15 | 0.53 | − | − |
| 5 | 1780 | 91 | 18 | 0.61 | + | − |
| 5 | 1781 | 83 | 25 | 0.4 | ++ | − |
| 5 | 1783 | 240 | 25 | 0.32 | + | − |
| 5 | 1785 | 345 | 15 | 0.8 | − | − |
| 5 | 1786 | 220 | 25 | 0.6 | − | + |
| 5 | 1787 | 106 | 15 | 0.8 | − | + |
| 5 | 1788 | 182 | 20 | 0.7 | − | − |
| 5 | 1789 | 183 | 15 | 0.73 | + | + |
| 5 | 1790 | 318 | 10 | 0.8 | + | + |
| 5 | 1791 | 331 | 25 | 0.48 | ++ | ++ |
| 5 | 1794 | 242 | 18 | 0.5 | + | − |
| 5 | 1795 | 150 | 20 | 0.7 | + | − |
| 5 | 1796 | 158 | 25 | 0.6 | − | − |
| 5 | 1797 | 174 | 10 | 0.8 | + | − |
| 5 | 1799 | 99 | 35 | 0.51 | − | − |
| 5 | 1801 | 185 | 25 | 0.4 | + | − |
| 5 | 1802 | 227 | 28 | 0.39 | ++ | − |
| 5 | 1804 | 341 | 18 | 0.5 | + | − |
| 5 | 1805 | 339 | 35 | 0.4 | + | ++ |
| 5 | 1807 | 324 | 18 | 0.5 | ++ | − |
| 5 | 1808 | 342 | 22 | 0.41 | + | − |
| 5 | 1809 | 87 | 20 | 0.4 | ++ | − |
| 5 | 1811 | 359 | 22 | 0.68 | − | − |
| 5 | 1812 | 350 | 22 | 0.41 | + | ++ |
| 5 | 1813 | 132 | 15 | 0.4 | + | + |
| 5 | 1814 | 146 | 30 | 0.6 | − | − |
| 5 | 1815 | 315 | 22 | 0.5 | + | − |
| 5 | 1816 | 370 | 22 | 0.41 | + | − |
| 10 | 1962 | 366 | 25 | 0.4 | +++ | ++ |
| 10 | 1964 | 221 | 28 | 0.29 | + | + |
| 10 | 1965 | 235 | 20 | 0.5 | + | − |
| 10 | 1969 | 263 | 18 | 0.78 | + | − |
| 10 | 1970 | 369 | 25 | 0.64 | − | +++ |
| 10 | 1971 | 180 | 25 | 0.48 | + | ++ |
| 10 | 1974 | 287 | 15 | 0.67 | +++ | + |
| 10 | 1975 | 170 | 15 | 0.53 | + | − |
| 10 | 1976 | 323 | 30 | 0.5 | ++ | − |
| 10 | 1977 | 381 | 40 | 0.4 | + | − |
| 10 | 1979 | 149 | 10 | 0.9 | + | ++ |
| 10 | 1980 | 104 | 30 | 0.4 | ++ | + |
| 10 | 1983 | 103 | 10 | 0.5 | ++ | + |
| 10 | 1984 | 215 | 18 | 0.72 | + | ++ |
| 10 | 1985 | 134 | 28 | 0.64 | − | − |
| 10 | 1986 | 138 | 18 | 0.39 | + | ++ |
| 10 | 1987 | 356 | 28 | 0.5 | + | − |
| 10 | 1988 | 305 | 22 | 0.59 | +++ | − |
| 10 | 1989 | 266 | 20 | 0.8 | − | ++ |
| 10 | 1990 | 142 | 22 | 0.82 | − | ++ |
| 10 | 1991 | 218 | 15 | 0.93 | − | − |
| 10 | 1997 | 303 | 18 | 0.83 | + | + |
| 10 | 1998 | 211 | 15 | 0.87 | + | + |
| 10 | 2002 | 209 | 18 | 0.78 | − | − |
| 10 | 2004 | 239 | 15 | 0.27 | ++ | + |
| 10 | 2006 | 373 | 20 | 0.75 | + | + |
| 10 | 2007 | 157 | 10 | 0.8 | ++ | ++ |
| 10 | 2008 | 113 | 18 | 0.39 | +++ | ++ |
| 10 | 2011 | 151 | 15 | 0.93 | + | + |
| 10 | 2015 | 322 | 40 | 0.6 | − | − |
| 10 | 2017 | 133 | 15 | 0.67 | + | + |
| 10 | 2019 | 172 | 20 | 0.65 | + | + |
| 10 | 2021 | 205 | 10 | 0.8 | + | ++ |
| 10 | 2022 | 124 | 28 | 0.61 | − | + |
| 10 | 2025 | 219 | 18 | 0.61 | ++ | − |
| 10 | 2026 | 213 | 15 | 0.93 | − | + |
| 10 | 2033 | 383 | 28 | 0.5 | + | + |
| 10 | 2034 | 293 | 18 | 0.89 | − | + |
| 10 | 2036 | 188 | 22 | 0.68 | + | − |
| 10 | 2038 | 179 | 15 | 0.73 | + | ++ |
| 10 | 2039 | 181 | 10 | 0.8 | ++ | +++ |
| 10 | 2043 | 375 | 18 | 0.67 | − | + |
| 10 | 2044 | 260 | 25 | 0.48 | ++ | ++ |
| 10 | 2048 | 155 | 20 | 0.75 | − | ++ |
| 10 | 2050 | 128 | 20 | 0.6 | + | ++ |
| 10 | 2051 | 175 | 25 | 0.64 | + | + |
| 10 | 2052 | 243 | 18 | 0.67 | ++ | + |
| 10 | 2054 | 307 | 20 | 0.65 | + | − |
| 10 | 2055 | 102 | 15 | 0.53 | ++ | + |
| 10 | 2056 | 329 | 15 | 0.67 | + | − |
| 10 | 2057 | 286 | 30 | 0.5 | +++ | + |
| 10 | 2058 | 145 | 10 | 0.6 | ++ | ++ |
| 10 | 2060 | 290 | 20 | 0.7 | ++ | + |
| 10 | 2061 | 321 | 10 | 0.8 | + | − |
| 10 | 2062 | 186 | 20 | 0.3 | + | ++ |
| 10 | 2065 | 283 | 18 | 0.67 | + | ++ |
| 10 | 2066 | 384 | 10 | 0.5 | + | + |
| 10 | 2067 | 223 | 15 | 0.8 | + | + |
| 10 | 2069 | 288 | 20 | 0.6 | ++++ | + |
| 10 | 2070 | 361 | 15 | 0.6 | +++ | + |
| 10 | 2072 | 216 | 25 | 0.6 | + | − |
| 10 | 2073 | 278 | 20 | 0.5 | ++ | ++ |
| 10 | 2074 | 246 | 15 | 0.6 | + | + |
| 10 | 2075 | 291 | 28 | 0.64 | + | − |
| 10 | 2077 | 364 | 15 | 0.67 | + | + |
| 10 | 2078 | 229 | 10 | 0.6 | + | ++ |
| 10 | 2079 | 184 | 15 | 0.73 | + | − |
| 10 | 2080 | 169 | 10 | 0.9 | + | + |
| 10 | 2082 | 161 | 20 | 0.85 | − | ++ |
| 10 | 2085 | 379 | 10 | 0.6 | + | − |
| 10 | 2087 | 137 | 15 | 0.67 | + | − |
| 10 | 2089 | 231 | 22 | 0.64 | + | + |
| 10 | 2091 | 190 | 15 | 0.87 | + | − |
| 10 | 2093 | 353 | 20 | 0.4 | + | + |
| 10 | 2094 | 166 | 18 | 0.78 | + | ++ |
| 10 | 2095 | 141 | 10 | 0.7 | + | + |
| 10 | 2097 | 348 | 20 | 0.8 | − | − |
| 10 | 2105 | 249 | 18 | 0.61 | ++ | + |
| 10 | 2108 | 140 | 18 | 0.89 | − | ++ |
| 10 | 2110 | 376 | 22 | 0.5 | ++ | + |
| 10 | 2111 | 269 | 15 | 0.87 | + | ++ |
| 10 | 2113 | 257 | 18 | 0.39 | ++ | − |
| 10 | 2114 | 131 | 22 | 0.59 | ++ | − |
| 10 | 2115 | 312 | 18 | 0.83 | − | ++ |
| 10 | 2116 | 198 | 20 | 0.9 | − | + |
| 10 | 2118 | 280 | 15 | 0.6 | + | + |
| 10 | 2119 | 125 | 22 | 0.64 | ++ | − |
| 10 | 2120 | 334 | 18 | 0.78 | − | + |
| 10 | 2121 | 232 | 28 | 0.61 | + | ++ |
| 10 | 2123 | 92 | 30 | 0.5 | + | − |
| 10 | 2125 | 355 | 35 | 0.4 | + | − |
| 10 | 2127 | 325 | 15 | 0.4 | +++ | ++ |
| 10 | 2129 | 139 | 25 | 0.72 | + | − |
| 10 | 2131 | 114 | 18 | 0.78 | + | ++ |
| 10 | 2132 | 234 | 20 | 0.5 | + | + |
| 10 | 2137 | 135 | 20 | 0.85 | − | ++ |
| 10 | 2138 | 108 | 10 | 0.7 | + | + |
| 10 | 2142 | 349 | 23 | 0.65 | + | + |
| 10 | 2143 | 330 | 30 | 0.4 | +++ | ++ |
| 10 | 2144 | 119 | 30 | 0.4 | + | − |
| 10 | 2145 | 252 | 35 | 0.51 | − | − |
| 10 | 2148 | 241 | 10 | 0.6 | + | + |
| 10 | 2149 | 338 | 20 | 0.4 | + | + |
| 10 | 2150 | 115 | 15 | 0.67 | + | + |
| 10 | 2156 | 107 | 15 | 0.4 | + | + |
| 10 | 2157 | 154 | 22 | 0.73 | + | + |
| 10 | 2158 | 127 | 22 | 0.5 | + | + |
| 10 | 2159 | 253 | 30 | 0.3 | ++++ | − |
| 10 | 2160 | 80 | 18 | 0.28 | + | ++ |
| 10 | 2161 | 197 | 18 | 0.83 | − | ++ |
| 10 | 2162 | 371 | 40 | 0.3 | ++ | − |
| 10 | 2163 | 111 | 35 | 0.4 | + | + |
| 10 | 2164 | 228 | 28 | 0.39 | +++ | ++ |
| 10 | 2165 | 192 | 28 | 0.5 | + | − |
| 10 | 2166 | 367 | 22 | 0.32 | + | − |
| 10 | 2167 | 251 | 35 | 0.29 | + | ++ |
| 10 | 2168 | 292 | 30 | 0.7 | − | +++ |
| 10 | 2171 | 225 | 22 | 0.64 | + | − |
| 10 | 2173 | 159 | 20 | 0.65 | + | − |
| 10 | 2174 | 282 | 18 | 0.72 | ++ | − |
| 10 | 2175 | 116 | 18 | 0.72 | + | ++ |
| 10 | 2177 | 279 | 15 | 0.53 | ++ | + |
| 10 | 2179 | 91 | 18 | 0.61 | + | + |
| 10 | 2180 | 83 | 25 | 0.4 | + | + |
| 10 | 2182 | 240 | 25 | 0.32 | ++ | + |
| 10 | 2183 | 270 | 20 | 0.6 | ++ | ++ |
| 10 | 2184 | 345 | 15 | 0.8 | − | + |
| 10 | 2185 | 220 | 25 | 0.6 | − | − |
| 10 | 2186 | 106 | 15 | 0.8 | + | − |
| 10 | 2187 | 182 | 20 | 0.7 | + | − |
| 10 | 2188 | 183 | 15 | 0.73 | + | − |
| 10 | 2189 | 318 | 10 | 0.8 | + | ++ |
| 10 | 2190 | 331 | 25 | 0.48 | ++ | ++ |
| 10 | 2193 | 242 | 18 | 0.5 | + | + |
| 10 | 2194 | 150 | 20 | 0.7 | + | ++ |
| 10 | 2195 | 158 | 25 | 0.6 | + | − |
| 10 | 2196 | 174 | 10 | 0.8 | + | − |
| 10 | 2198 | 99 | 35 | 0.51 | + | − |
| 10 | 2200 | 185 | 25 | 0.4 | + | − |
| 10 | 2201 | 227 | 28 | 0.39 | +++ | ++ |
| 10 | 2203 | 341 | 18 | 0.5 | +++ | ++ |
| 10 | 2204 | 339 | 35 | 0.4 | + | − |
| 10 | 2206 | 324 | 18 | 0.5 | ++++ | − |
| 10 | 2207 | 342 | 22 | 0.41 | ++++ | − |
| 10 | 2208 | 87 | 20 | 0.4 | ++ | − |
| 10 | 2210 | 359 | 22 | 0.68 | + | − |
| 10 | 2211 | 350 | 22 | 0.41 | + | + |
| 10 | 2212 | 132 | 15 | 0.4 | + | − |
| 10 | 2213 | 146 | 30 | 0.6 | − | − |
| 10 | 2214 | 315 | 22 | 0.5 | + | ++ |
| 10 | 2215 | 370 | 22 | 0.41 | ++ | − |
| 16 | 2371 | 366 | 25 | 0.4 | + | + |
| 16 | 2373 | 221 | 28 | 0.29 | − | − |
| 16 | 2374 | 235 | 20 | 0.5 | + | ++ |
| 16 | 2378 | 263 | 18 | 0.78 | − | − |
| 16 | 2380 | 180 | 25 | 0.48 | + | − |
| 16 | 2383 | 287 | 15 | 0.67 | + | + |
| 16 | 2384 | 170 | 15 | 0.53 | + | − |
| 16 | 2385 | 323 | 30 | 0.5 | + | ++ |
| 16 | 2386 | 381 | 40 | 0.4 | − | + |
| 16 | 2388 | 149 | 10 | 0.9 | + | + |
| 16 | 2389 | 104 | 30 | 0.4 | + | − |
| 16 | 2392 | 103 | 10 | 0.5 | + | ++ |
| 16 | 2393 | 215 | 18 | 0.72 | + | ++ |
| 16 | 2394 | 134 | 28 | 0.64 | − | − |
| 16 | 2395 | 138 | 18 | 0.39 | + | − |
| 16 | 2396 | 356 | 28 | 0.5 | + | + |
| 16 | 2397 | 305 | 22 | 0.59 | + | + |
| 16 | 2398 | 266 | 20 | 0.8 | − | ++ |
| 16 | 2399 | 142 | 22 | 0.82 | − | − |
| 16 | 2400 | 218 | 15 | 0.93 | − | ++ |
| 16 | 2406 | 303 | 18 | 0.83 | + | + |
| 16 | 2407 | 211 | 15 | 0.87 | + | − |
| 16 | 2411 | 209 | 18 | 0.78 | − | + |
| 16 | 2413 | 239 | 15 | 0.27 | + | ++ |
| 16 | 2415 | 373 | 20 | 0.75 | − | + |
| 16 | 2416 | 157 | 10 | 0.8 | + | + |
| 16 | 2417 | 113 | 18 | 0.39 | + | + |
| 16 | 2420 | 151 | 15 | 0.93 | − | − |
| 16 | 2426 | 133 | 15 | 0.67 | + | ++ |
| 16 | 2428 | 172 | 20 | 0.65 | − | − |
| 16 | 2430 | 205 | 10 | 0.8 | + | ++ |
| 16 | 2431 | 124 | 28 | 0.61 | + | + |
| 16 | 2434 | 219 | 18 | 0.61 | + | + |
| 16 | 2435 | 213 | 15 | 0.93 | − | − |
| 16 | 2442 | 383 | 28 | 0.5 | + | ++ |
| 16 | 2443 | 293 | 18 | 0.89 | − | − |
| 16 | 2445 | 188 | 22 | 0.68 | + | − |
| 16 | 2447 | 179 | 15 | 0.73 | + | + |
| 16 | 2448 | 181 | 10 | 0.8 | + | + |
| 16 | 2452 | 375 | 18 | 0.67 | − | ++ |
| 16 | 2453 | 260 | 25 | 0.48 | + | ++ |
| 16 | 2457 | 155 | 20 | 0.75 | − | + |
| 16 | 2459 | 128 | 20 | 0.6 | + | + |
| 16 | 2461 | 243 | 18 | 0.67 | + | ++ |
| 16 | 2463 | 307 | 20 | 0.65 | + | + |
| 16 | 2464 | 102 | 15 | 0.53 | + | + |
| 16 | 2465 | 329 | 15 | 0.67 | + | + |
| 16 | 2466 | 286 | 30 | 0.5 | + | ++ |
| 16 | 2467 | 145 | 10 | 0.6 | + | − |
| 16 | 2469 | 290 | 20 | 0.7 | + | ++ |
| 16 | 2470 | 321 | 10 | 0.8 | − | ++ |
| 16 | 2471 | 186 | 20 | 0.3 | − | − |
| 16 | 2474 | 283 | 18 | 0.67 | + | ++ |
| 16 | 2475 | 384 | 10 | 0.5 | + | + |
| 16 | 2476 | 223 | 15 | 0.8 | − | − |
| 16 | 2478 | 288 | 20 | 0.6 | + | − |
| 16 | 2479 | 361 | 15 | 0.6 | + | ++ |
| 16 | 2481 | 216 | 25 | 0.6 | + | − |
| 16 | 2482 | 278 | 20 | 0.5 | + | − |
| 16 | 2483 | 246 | 15 | 0.6 | + | − |
| 16 | 2484 | 291 | 28 | 0.64 | − | ++ |
| 16 | 2486 | 364 | 15 | 0.67 | + | + |
| 16 | 2487 | 229 | 10 | 0.6 | + | − |
| 16 | 2488 | 184 | 15 | 0.73 | + | + |
| 16 | 2491 | 161 | 20 | 0.85 | − | ++ |
| 16 | 2494 | 379 | 10 | 0.6 | + | + |
| 16 | 2496 | 137 | 15 | 0.67 | + | ++ |
| 16 | 2498 | 231 | 22 | 0.64 | + | + |
| 16 | 2500 | 190 | 15 | 0.87 | − | + |
| 16 | 2502 | 353 | 20 | 0.4 | + | − |
| 16 | 2503 | 166 | 18 | 0.78 | − | ++ |
| 16 | 2504 | 141 | 10 | 0.7 | − | + |
| 16 | 2506 | 348 | 20 | 0.8 | − | − |
| 16 | 2514 | 249 | 18 | 0.61 | + | − |
| 16 | 2516 | 363 | 22 | 0.59 | − | +++ |
| 16 | 2519 | 376 | 22 | 0.5 | + | − |
| 16 | 2520 | 269 | 15 | 0.87 | + | + |
| 16 | 2522 | 257 | 18 | 0.39 | + | ++ |
| 16 | 2523 | 131 | 22 | 0.59 | + | − |
| 16 | 2524 | 312 | 18 | 0.83 | − | + |
| 16 | 2525 | 198 | 20 | 0.9 | − | − |
| 16 | 2527 | 280 | 15 | 0.6 | + | + |
| 16 | 2528 | 125 | 22 | 0.64 | + | + |
| 16 | 2529 | 334 | 18 | 0.78 | − | + |
| 16 | 2530 | 232 | 28 | 0.61 | + | ++ |
| 16 | 2532 | 92 | 30 | 0.5 | + | + |
| 16 | 2534 | 355 | 35 | 0.4 | + | ++ |
| 16 | 2536 | 325 | 15 | 0.4 | − | ++ |
| 16 | 2538 | 139 | 25 | 0.72 | − | − |
| 16 | 2540 | 114 | 18 | 0.78 | − | ++++ |
| 16 | 2541 | 234 | 20 | 0.5 | + | + |
| 16 | 2546 | 135 | 20 | 0.85 | − | ++++ |
| 16 | 2547 | 108 | 10 | 0.7 | + | ++ |
| 16 | 2551 | 349 | 23 | 0.65 | − | − |
| 16 | 2552 | 330 | 30 | 0.4 | + | ++ |
| 16 | 2553 | 119 | 30 | 0.4 | + | − |
| 16 | 2554 | 252 | 35 | 0.51 | + | − |
| 16 | 2557 | 241 | 10 | 0.6 | − | − |
| 16 | 2558 | 338 | 20 | 0.4 | + | ++ |
| 16 | 2559 | 115 | 15 | 0.67 | + | + |
| 16 | 2565 | 107 | 15 | 0.4 | + | + |
| 16 | 2566 | 154 | 22 | 0.73 | + | − |
| 16 | 2567 | 127 | 22 | 0.5 | + | + |
| 16 | 2568 | 253 | 30 | 0.3 | + | + |
| 16 | 2569 | 80 | 18 | 0.28 | + | + |
| 16 | 2570 | 197 | 18 | 0.83 | − | ++ |
| 16 | 2571 | 371 | 40 | 0.3 | + | + |
| 16 | 2572 | 111 | 35 | 0.4 | + | ++ |
| 16 | 2573 | 228 | 28 | 0.39 | + | ++ |
| 16 | 2574 | 192 | 28 | 0.5 | + | ++ |
| 16 | 2575 | 367 | 22 | 0.32 | + | + |
| 16 | 2576 | 251 | 35 | 0.29 | + | + |
| 16 | 2577 | 292 | 30 | 0.7 | − | − |
| 16 | 2582 | 159 | 20 | 0.65 | + | − |
| 16 | 2583 | 282 | 18 | 0.72 | + | + |
| 16 | 2584 | 116 | 18 | 0.72 | + | + |
| 16 | 2586 | 279 | 15 | 0.53 | + | + |
| 16 | 2587 | 337 | 28 | 0.39 | − | +++ |
| 16 | 2588 | 91 | 18 | 0.61 | + | + |
| 16 | 2589 | 83 | 25 | 0.4 | + | + |
| 16 | 2590 | 143 | 22 | 0.86 | − | ++ |
| 16 | 2591 | 240 | 25 | 0.32 | + | + |
| 16 | 2592 | 270 | 20 | 0.6 | + | − |
| 16 | 2593 | 345 | 15 | 0.8 | − | + |
| 16 | 2594 | 220 | 25 | 0.6 | − | − |
| 16 | 2595 | 106 | 15 | 0.8 | − | ++ |
| 16 | 2596 | 182 | 20 | 0.7 | + | − |
| 16 | 2597 | 183 | 15 | 0.73 | + | − |
| 16 | 2598 | 318 | 10 | 0.8 | + | + |
| 16 | 2599 | 331 | 25 | 0.48 | + | + |
| 16 | 2602 | 242 | 18 | 0.5 | + | − |
| 16 | 2603 | 150 | 20 | 0.7 | + | + |
| 16 | 2604 | 158 | 25 | 0.6 | + | − |
| 16 | 2605 | 174 | 10 | 0.8 | + | − |
| 16 | 2606 | 327 | 20 | 0.8 | − | − |
| 16 | 2609 | 185 | 25 | 0.4 | + | + |
| 16 | 2610 | 227 | 28 | 0.39 | + | + |
| 16 | 2612 | 341 | 18 | 0.5 | + | ++ |
| 16 | 2613 | 339 | 35 | 0.4 | + | ++ |
| 16 | 2615 | 324 | 18 | 0.5 | + | + |
| 16 | 2616 | 342 | 22 | 0.41 | + | − |
| 16 | 2617 | 87 | 20 | 0.4 | + | + |
| 16 | 2619 | 359 | 22 | 0.68 | − | ++ |
| 16 | 2620 | 350 | 22 | 0.41 | + | − |
| 16 | 2621 | 132 | 15 | 0.4 | + | ++ |
| 16 | 2622 | 146 | 30 | 0.6 | − | − |
| 16 | 2623 | 315 | 22 | 0.5 | + | + |
| 16 | 2624 | 370 | 22 | 0.41 | + | + |

TABLE 65
dual prime editing pegRNAs tested with single prime editing system

| | pegRNA | RTT | | | % | % |
|---|---|---|---|---|---|---|
| 3′ | SEQ ID | pairing | RTT | RTT | editing | indel |
| spacer | NO: | No. | length | GC % | efficiency | frequency |
| 5 | 1818 | 84 | 25 | 0.6 | − | ++ |
| 5 | 1819 | 85 | 15 | 0.8 | − | ++ |
| 5 | 1819 | 85 | 15 | 0.8 | − | ++ |
| 5 | 1819 | 85 | 15 | 0.8 | − | +++ |
| 5 | 1820 | 86 | 25 | 0.48 | − | − |
| 5 | 1820 | 86 | 25 | 0.48 | − | + |
| 5 | 1820 | 86 | 25 | 0.48 | − | − |
| 5 | 1821 | 88 | 30 | 0.4 | + | + |
| 5 | 1822 | 89 | 25 | 0.4 | + | − |
| 5 | 1822 | 89 | 25 | 0.4 | + | − |
| 5 | 1823 | 90 | 20 | 0.8 | − | − |
| 5 | 1823 | 90 | 20 | 0.8 | − | − |
| 5 | 1823 | 90 | 20 | 0.8 | − | ++ |
| 5 | 1826 | 96 | 15 | 0.6 | ++ | − |
| 5 | 1826 | 96 | 15 | 0.6 | +++ | − |
| 5 | 1826 | 96 | 15 | 0.6 | ++ | + |
| 5 | 1827 | 112 | 15 | 0.67 | + | + |
| 5 | 1827 | 112 | 15 | 0.67 | + | + |
| 5 | 1827 | 112 | 15 | 0.67 | + | + |
| 5 | 1828 | 121 | 20 | 0.5 | + | − |
| 5 | 1829 | 122 | 15 | 0.4 | ++ | + |
| 5 | 1829 | 122 | 15 | 0.4 | + | − |
| 5 | 1829 | 122 | 15 | 0.4 | ++ | + |
| 5 | 1830 | 123 | 20 | 0.6 | − | − |
| 5 | 1830 | 123 | 20 | 0.6 | − | − |
| 5 | 1831 | 129 | 15 | 0.53 | +++ | − |
| 5 | 1831 | 129 | 15 | 0.53 | ++ | + |
| 5 | 1831 | 129 | 15 | 0.53 | ++++ | − |
| 5 | 1832 | 130 | 30 | 0.5 | + | + |
| 5 | 1832 | 130 | 30 | 0.5 | + | + |
| 5 | 1832 | 130 | 30 | 0.5 | ++ | − |
| 5 | 1836 | 206 | 30 | 0.4 | + | − |
| 5 | 1840 | 306 | 20 | 0.7 | + | − |
| 5 | 1840 | 306 | 20 | 0.7 | − | ++ |
| 5 | 1843 | 319 | 20 | 0.6 | + | + |
| 5 | 1845 | 340 | 25 | 0.4 | + | ++ |
| 5 | 1846 | 368 | 20 | 0.5 | ++ | ++ |
| 5 | 1847 | 380 | 20 | 0.5 | + | − |
| 5 | 1848 | 382 | 20 | 0.4 | ++ | − |
| 5 | 1848 | 382 | 20 | 0.4 | ++ | − |
| 5 | 1848 | 382 | 20 | 0.4 | +++ | − |
| 10 | 2217 | 85 | 15 | 0.8 | + | ++ |
| 10 | 2217 | 85 | 15 | 0.8 | − | ++ |
| 10 | 2218 | 88 | 30 | 0.4 | +++ | + |
| 10 | 2218 | 88 | 30 | 0.4 | +++ | ++ |
| 10 | 2219 | 90 | 20 | 0.8 | − | + |
| 10 | 2223 | 96 | 15 | 0.6 | +++ | ++ |
| 10 | 2223 | 96 | 15 | 0.6 | +++ | ++ |
| 10 | 2223 | 96 | 15 | 0.6 | +++ | − |
| 10 | 2224 | 110 | 20 | 0.7 | − | + |
| 10 | 2224 | 110 | 20 | 0.7 | − | − |
| 10 | 2225 | 112 | 15 | 0.67 | + | ++ |
| 10 | 2225 | 112 | 15 | 0.67 | ++ | + |
| 10 | 2225 | 112 | 15 | 0.67 | ++ | + |
| 10 | 2226 | 122 | 15 | 0.4 | ++ | + |
| 10 | 2227 | 123 | 20 | 0.6 | − | − |
| 10 | 2227 | 123 | 20 | 0.6 | − | − |
| 10 | 2228 | 129 | 15 | 0.53 | +++ | ++ |
| 10 | 2228 | 129 | 15 | 0.53 | +++ | ++ |
| 10 | 2228 | 129 | 15 | 0.53 | +++ | ++ |
| 10 | 2229 | 130 | 30 | 0.5 | ++ | ++ |
| 10 | 2229 | 130 | 30 | 0.5 | + | − |
| 10 | 2229 | 130 | 30 | 0.5 | +++ | − |
| 10 | 2230 | 144 | 30 | 0.4 | ++ | ++ |
| 10 | 2234 | 247 | 25 | 0.4 | + | ++ |
| 10 | 2234 | 247 | 25 | 0.4 | ++ | ++ |
| 10 | 2234 | 247 | 25 | 0.4 | + | − |
| 10 | 2236 | 272 | 25 | 0.6 | + | ++ |
| 10 | 2238 | 306 | 20 | 0.7 | ++ | + |
| 10 | 2241 | 319 | 20 | 0.6 | ++ | + |
| 10 | 2242 | 351 | 25 | 0.48 | ++ | ++ |
| 10 | 2242 | 351 | 25 | 0.48 | ++ | + |
| 10 | 2242 | 351 | 25 | 0.48 | +++ | − |
| 10 | 2243 | 354 | 15 | 0.4 | ++ | − |
| 10 | 2243 | 354 | 15 | 0.4 | ++ | ++ |
| 10 | 2244 | 368 | 20 | 0.5 | +++ | + |
| 10 | 2244 | 368 | 20 | 0.5 | +++ | − |
| 10 | 2245 | 378 | 15 | 0.8 | ++ | ++ |
| 10 | 2246 | 380 | 20 | 0.5 | + | ++ |
| 10 | 2247 | 382 | 20 | 0.4 | ++ | ++ |
| 10 | 2247 | 382 | 20 | 0.4 | ++ | ++ |
| 10 | 2247 | 382 | 20 | 0.4 | ++ | + |
| 16 | 2626 | 81 | 15 | 0.4 | + | ++ |
| 16 | 2627 | 86 | 25 | 0.48 | − | ++ |
| 16 | 2627 | 86 | 25 | 0.48 | − | ++ |
| 16 | 2628 | 88 | 30 | 0.4 | + | + |
| 16 | 2629 | 89 | 25 | 0.4 | + | ++ |
| 16 | 2629 | 89 | 25 | 0.4 | + | − |
| 16 | 2629 | 89 | 25 | 0.4 | + | ++ |
| 16 | 2630 | 90 | 20 | 0.8 | − | − |
| 16 | 2630 | 90 | 20 | 0.8 | − | ++ |
| 16 | 2630 | 90 | 20 | 0.8 | − | ++ |
| 16 | 2633 | 96 | 15 | 0.6 | + | ++ |
| 16 | 2633 | 96 | 15 | 0.6 | + | − |
| 16 | 2635 | 105 | 15 | 0.8 | + | ++ |
| 16 | 2636 | 109 | 20 | 0.4 | + | + |
| 16 | 2636 | 109 | 20 | 0.4 | + | ++ |
| 16 | 2636 | 109 | 20 | 0.4 | + | ++ |
| 16 | 2637 | 123 | 20 | 0.6 | − | + |
| 16 | 2637 | 123 | 20 | 0.6 | − | + |
| 16 | 2638 | 126 | 15 | 0.4 | + | + |
| 16 | 2638 | 126 | 15 | 0.4 | + | + |
| 16 | 2639 | 129 | 15 | 0.53 | + | − |
| 16 | 2639 | 129 | 15 | 0.53 | + | + |
| 16 | 2639 | 129 | 15 | 0.53 | + | − |
| 16 | 2640 | 130 | 30 | 0.5 | + | ++ |
| 16 | 2640 | 130 | 30 | 0.5 | + | ++ |
| 16 | 2640 | 130 | 30 | 0.5 | + | − |
| 16 | 2644 | 206 | 30 | 0.4 | + | ++ |
| 16 | 2646 | 250 | 15 | 0.6 | − | ++ |
| 16 | 2647 | 259 | 30 | 0.4 | + | + |
| 16 | 2648 | 300 | 25 | 0.6 | − | ++ |
| 16 | 2648 | 300 | 25 | 0.6 | + | − |
| 16 | 2648 | 300 | 25 | 0.6 | + | ++ |
| 16 | 2649 | 306 | 20 | 0.7 | + | + |
| 16 | 2649 | 306 | 20 | 0.7 | + | ++ |
| 16 | 2649 | 306 | 20 | 0.7 | + | ++ |
| 16 | 2651 | 319 | 20 | 0.6 | + | − |
| 16 | 2652 | 320 | 15 | 0.67 | + | + |
| 16 | 2652 | 320 | 15 | 0.67 | + | − |
| 16 | 2652 | 320 | 15 | 0.67 | + | + |
| 16 | 2654 | 351 | 25 | 0.48 | + | ++ |
| 16 | 2655 | 368 | 20 | 0.5 | + | − |
| 16 | 2656 | 378 | 15 | 0.8 | − | + |
| 16 | 2657 | 380 | 20 | 0.5 | − | + |
| 16 | 2657 | 380 | 20 | 0.5 | − | − |

TABLE 66
dual prime editing pegRNAs tested with single prime editing system

| 3′ | pegRNA | RTT | | | % | % |
|---|---|---|---|---|---|---|
| | SEQ ID | pairing | RTT | RTT | editing | indel |
| spacer | NO: | No. | length | GC % | efficiency | frequency |
| 5 | 1849 | 390 | 12 | 0.5 | + | − |
| 5 | 1850 | 390 | 12 | 0.5 | − | − |
| 5 | 1851 | 390 | 12 | 0.5 | + | + |
| 5 | 1852 | 390 | 12 | 0.5 | + | − |
| 5 | 1853 | 390 | 12 | 0.5 | − | +++ |
| 5 | 1854 | 392 | 12 | 0.916666667 | − | ++ |
| 5 | 1855 | 392 | 12 | 0.916666667 | + | − |
| 5 | 1859 | 391 | 12 | 0.583333333 | + | − |
| 5 | 1860 | 391 | 12 | 0.583333333 | + | − |
| 5 | 1861 | 391 | 12 | 0.583333333 | ++ | ++ |
| 5 | 1862 | 391 | 12 | 0.583333333 | ++ | ++ |
| 5 | 1863 | 391 | 12 | 0.583333333 | + | + |
| 10 | 2248 | 390 | 12 | 0.5 | ++ | +++ |
| 10 | 2249 | 390 | 12 | 0.5 | ++ | + |
| 10 | 2250 | 390 | 12 | 0.5 | ++ | − |
| 10 | 2251 | 390 | 12 | 0.5 | + | − |
| 10 | 2252 | 390 | 12 | 0.5 | + | − |
| 10 | 2253 | 392 | 12 | 0.916666667 | − | ++ |
| 10 | 2254 | 392 | 12 | 0.916666667 | − | ++ |
| 10 | 2255 | 392 | 12 | 0.916666667 | − | ++ |
| 10 | 2258 | 391 | 12 | 0.583333333 | + | − |
| 10 | 2259 | 391 | 12 | 0.583333333 | + | ++ |
| 10 | 2260 | 391 | 12 | 0.583333333 | ++ | ++ |
| 10 | 2261 | 391 | 12 | 0.583333333 | ++++ | + |
| 10 | 2262 | 391 | 12 | 0.583333333 | + | − |
| 16 | 2658 | 390 | 12 | 0.5 | − | − |
| 16 | 2659 | 390 | 12 | 0.5 | + | ++ |
| 16 | 2660 | 390 | 12 | 0.5 | + | − |
| 16 | 2661 | 390 | 12 | 0.5 | + | + |
| 16 | 2662 | 390 | 12 | 0.5 | − | − |
| 16 | 2663 | 392 | 12 | 0.916666667 | + | ++ |
| 16 | 2664 | 392 | 12 | 0.916666667 | − | − |
| 16 | 2668 | 391 | 12 | 0.583333333 | + | − |
| 16 | 2669 | 391 | 12 | 0.583333333 | + | − |
| 16 | 2670 | 391 | 12 | 0.583333333 | + | + |
| 16 | 2671 | 391 | 12 | 0.583333333 | + | − |
| 16 | 2672 | 391 | 12 | 0.583333333 | + | + |

Table 67 provides sequences referenced throughout the present disclosure.

TABLE 67
amino acid and nucleic acid sequences

| SEQ | | |
|---|---|---|
| ID NO: | Description | SEQUENCE |
| 3577 | WT M-MLV RT | TLNIEDEHRLHETSKEPDVSLGSTWLSDFPQAWAETGGMGLAVRQAPLIIPLKATST |
| | | PVSIKQYPMSQEARLGIKPHIQRLLDQGILVPCQSPWNTPLLPVKKPGTNDYRPVQD |
| | | LREVNKRVEDIHPTVPNPYNLLSGLPPSHQWYTVLDLKDAFFCLRLHPTSQPLFAFE |
| | | WRDPEMGISGQLTWTRLPQGFKNSPTLFDEALHRDLADFRIQHPDLILLQYVDDLLL |
| | | AATSELDCQQGTRALLQTLGNLGYRASAKKAQICQKQVKYLGYLLKEGQRWLTEA |
| | | RKETVMGQPTPKTPRQLREFLGTAGFCRLWIPGFAEMAAPLYPLTKTGTLFNWGPD |
| | | QQKAYQEIKQALLTAPALGLPDLTKPFELFVDEKQGYAKGVLTQKLGPWRRPVAYL |
| | | SKKLDPVAAGWPPCLRMVAAIAVLTKDAGKLTMGQPLVILAPHAVEALVKQPPDR |
| | | WLSNARMTHYQALLLDTDRVQFGPVVALNPATLLPLPEEGLQHNCLDILAEAHGTR |
| | | PDLTDQPLPDADHTWYTDGSSLLQEGQRKAGAAVTTETEVIWAKALPAGTSAQRA |
| | | ELIALTQALKMAEGKKLNVYTDSRYAFATAHIHGEIYRRRGLLTSEGKEIKNKDEIL |
| | | ALLKALFLPKRLSIIHCPGHQKGHSAEARGNRMADQAARKAAITETPDTSTLLIENSS |
| | | P |
| 3578 | Reference M-MLV | TLNIEDEYRLHETSKEPDVSLGSTWLSDFPQAWAETGGMGLAVRQAPLIIPLKATST |
| | RT; M-MLV | PVSIKQYPMSQEARLGIKPHIQRLLDQGILVPCQSPWNTPLLPVKKPGTNDYRPVQD |
| | (H8Y) RT | LREVNKRVEDIHPTVPNPYNLLSGLPPSHQWYTVLDLKDAFFCLRLHPTSQPLFAFE |
| | | WRDPEMGISGQLTWTRLPQGFKNSPTLFDEALHRDLADFRIQHPDLILLQYVDDLLL |
| | | AATSELDCQQGTRALLQTLGNLGYRASAKKAQICQKQVKYLGYLLKEGQRWLTEA |
| | | RKETVMGQPTPKTPRQLREFLGTAGFCRLWIPGFAEMAAPLYPLTKTGTLFNWGPD |
| | | QQKAYQEIKQALLTAPALGLPDLTKPFELFVDEKQGYAKGVLTQKLGPWRRPVAYL |
| | | SKKLDPVAAGWPPCLRMVAAIAVLTKDAGKLTMGQPLVILAPHAVEALVKQPPDR |
| | | WLSNARMTHYQALLLDTDRVQFGPVVALNPATLLPLPEEGLQHNCLDILAEAHGTR |
| | | PDLTDQPLPDADHTWYTDGSSLLQEGQRKAGAAVTTETEVIWAKALPAGTSAQRA |
| | | ELIALTQALKMAEGKKLNVYTDSRYAFATAHIHGEIYRRRGLLTSEGKEIKNKDEIL |
| | | ALLKALFLPKRLSIIHCPGHQKGHSAEARGNRMADQAARKAAITETPDTSTLLIENSS |
| | | P |
| 3579 | M-MLV RT | TLNIEDEYRLHETSKEPDVSLGSTWLSDFPQAWAETGGMGLAVRQAPLIIPLKATST |
| | (pentamutant | PVSIKQYPMSQEARLGIKPHIQRLLDQGILVPCQSPWNTPLLPVKKPGTNDYRPVQD |
| | compared to | LREVNKRVEDIHPTVPNPYNLLSGLPPSHQWYTVLDLKDAFFCLRLHPTSQPLFAFE |
| | Reference M-MLV) | WRDPEMGISGQLTWTRLPQGFKNSPTLFNEALHRDLADFRIQHPDLILLQYVDDLLL |
| | | AATSELDCQQGTRALLQTLGNLGYRASAKKAQICQKQVKYLGYLLKEGQRWLTEA |
| | | RKETVMGQPTPKTPRQLREFLGKAGFCRLFIPGFAEMAAPLYPLTKPGTLFNWGPDQ |
| | | QKAYQEIKQALLTAPALGLPDLTKPFELFVDEKQGYAKGVLTQKLGPWRRPVAYLS |
| | | KKLDPVAAGWPPCLRMVAAIAVLTKDAGKLTMGQPLVILAPHAVEALVKQPPDRW |
| | | LSNARMTHYQALLLDTDRVQFGPVVALNPATLLPLPEEGLQHNCLDILAEAHGTRP |
| | | DLTDQPLPDADHTWYTDGSSLLQEGQRKAGAAVTTETEVIWAKALPAGTSAQRAE |
| | | LIALTQALKMAEGKKLNVYTDSRYAFATAHIHGEIYRRRGWLTSEGKEIKNKDEILA |
| | | LLKALFLPKRLSIIHCPGHQKGHSAEARGNRMADQAARKAAITETPDTSTLLIENSSP |
| 3580 | Wild Type | MDKKYSIGLDIGTNSVGWAVITDEYKVPSKKFKVLGNTDRHSIKKNLIGALLFDSGE |
| | Streptococcus | TAEATRLKRTARRRYTRRKNRICYLQEIFSNEMAKVDDSFFHRLEESFLVEEDKKHE |
| | pyogenes Cas9 | RHPIFGNIVDEVAYHEKYPTIYHLRKKLVDSTDKADLRLIYLALAHMIKFRGHFLIEG |
| | (SpCas9) | DLNPDNSDVDKLFIQLVQTYNQLFEENPINASGVDAKAILSARLSKSRRLENLIAQLP |
| | | GEKKNGLFGNLIALSLGLTPNFKSNFDLAEDAKLQLSKDTYDDDLDNLLAQIGDQY |
| | | ADLFLAAKNLSDAILLSDILRVNTEITKAPLSASMIKRYDEHHQDLTLLKALVRQQLP |
| | | EKYKEIFFDQSKNGYAGYIDGGASQEEFYKFIKPILEKMDGTEELLVKLNREDLLRK |
| | | QRTFDNGSIPHQIHLGELHAILRRQEDFYPFLKDNREKIEKILTFRIPYYVGPLARGNS |
| | | RFAWMTRKSEETITPWNFEEVVDKGASAQSFIERMTNFDKNLPNEKVLPKHSLLYE |
| | | YFTVYNELTKVKYVTEGMRKPAFLSGEQKKAIVDLLFKTNRKVTVKQLKEDYFKKI |
| | | ECFDSVEISGVEDRFNASLGTYHDLLKIIKDKDFLDNEENEDILEDIVLTLTLFEDREM |
| | | IEERLKTYAHLFDDKVMKQLKRRRYTGWGRLSRKLINGIRDKQSGKTILDFLKSDGF |
| | | ANRNFMQLIHDDSLTFKEDIQKAQVSGQGDSLHEHIANLAGSPAIKKGILQTVKVVD |
| | | ELVKVMGRHKPENIVIEMARENQTTQKGQKNSRERMKRIEEGIKELGSQILKEHPVE |
| | | NTQLQNEKLYLYYLQNGRDMYVDQELDINRLSDYDVDHIVPQSFLKDDSIDNKVLT |
| | | RSDKNRGKSDNVPSEEVVKKMKNYWRQLLNAKLITQRKFDNLTKAERGGLSELDK |
| | | AGFIKRQLVETRQITKHVAQILDSRMNTKYDENDKLIREVKVITLKSKLVSDFRKDF |
| | | QFYKVREINNYHHAHDAYLNAVVGTALIKKYPKLESEFVYGDYKVYDVRKMIAKS |
| | | EQEIGKATAKYFFYSNIMNFFKTEITLANGEIRKRPLIETNGETGEIVWDKGRDFATV |
| | | RKVLSMPQVNIVKKTEVQTGGFSKESILPKRNSDKLIARKKDWDPKKYGGFDSPTV |
| | | AYSVLVVAKVEKGKSKKLKSVKELLGITIMERSSFEKNPIDFLEAKGYKEVKKDLIIK |
| | | LPKYSLFELENGRKRMLASAGELQKGNELALPSKYVNFLYLASHYEKLKGSPEDNE |
| | | QKQLFVEQHKHYLDEIIEQISEFSKRVILADANLDKVLSAYNKHRDKPIREQAENIIH |
| | | LFTLTNLGAPAAFKYFDTTIDRKRYTSTKEVLDATLIHQSITGLYETRIDLSQLGGD |
| 3581 | spCas9(H840A) | MDKKYSIGLDIGTNSVGWAVITDEYKVPSKKFKVLGNTDRHSIKKNLIGALLFDSGE |
| | Nickase | TAEATRLKRTARRRYTRRKNRICYLQEIFSNEMAKVDDSFFHRLEESFLVEEDKKHE |
| | | RHPIFGNIVDEVAYHEKYPTIYHLRKKLVDSTDKADLRLIYLALAHMIKFRGHFLIEG |
| | | DLNPDNSDVDKLFIQLVQTYNQLFEENPINASGVDAKAILSARLSKSRRLENLIAQLP |
| | | GEKKNGLFGNLIALSLGLTPNFKSNFDLAEDAKLQLSKDTYDDDLDNLLAQIGDQY |
| | | ADLFLAAKNLSDAILLSDILRVNTEITKAPLSASMIKRYDEHHQDLTLLKALVRQQLP |
| | | EKYKEIFFDQSKNGYAGYIDGGASQEEFYKFIKPILEKMDGTEELLVKLNREDLLRK |
| | | QRTFDNGSIPHQIHLGELHAILRRQEDFYPFLKDNREKIEKILTFRIPYYVGPLARGNS |
| | | RFAWMTRKSEETITPWNFEEVVDKGASAQSFIERMTNFDKNLPNEKVLPKHSLLYE |
| | | YFTVYNELTKVKYVTEGMRKPAFLSGEQKKAIVDLLFKTNRKVTVKQLKEDYFKKI |
| | | ECFDSVEISGVEDRFNASLGTYHDLLKIIKDKDFLDNEENEDILEDIVLTLTLFEDREM |
| | | IEERLKTYAHLFDDKVMKQLKRRRYTGWGRLSRKLINGIRDKQSGKTILDFLKSDGF |
| | | ANRNFMQLIHDDSLTFKEDIQKAQVSGQGDSLHEHIANLAGSPAIKKGILQTVKVVD |
| | | ELVKVMGRHKPENIVIEMARENQTTQKGQKNSRERMKRIEEGIKELGSQILKEHPVE |
| | | NTQLQNEKLYLYYLQNGRDMYVDQELDINRLSDYDVDAIVPQSFLKDDSIDNKVLT |
| | | RSDKNRGKSDNVPSEEVVKKMKNYWRQLLNAKLITQRKFDNLTKAERGGLSELDK |
| | | AGFIKRQLVETRQITKHVAQILDSRMNTKYDENDKLIREVKVITLKSKLVSDFRKDF |
| | | QFYKVREINNYHHAHDAYLNAVVGTALIKKYPKLESEFVYGDYKVYDVRKMIAKS |
| | | EQEIGKATAKYFFYSNIMNFFKTEITLANGEIRKRPLIETNGETGEIVWDKGRDFATV |
| | | RKVLSMPQVNIVKKTEVQTGGFSKESILPKRNSDKLIARKKDWDPKKYGGFDSPTV |
| | | AYSVLVVAKVEKGKSKKLKSVKELLGITIMERSSFEKNPIDFLEAKGYKEVKKDLIIK |
| | | LPKYSLFELENGRKRMLASAGELQKGNELALPSKYVNFLYLASHYEKLKGSPEDNE |
| | | QKQLFVEQHKHYLDEIIEQISEFSKRVILADANLDKVLSAYNKHRDKPIREQAENIIH |
| | | LFTLTNLGAPAAFKYFDTTIDRKRYTSTKEVLDATLIHQSITGLYETRIDLSQLGGD |
| 3582 | Met(−) | DKKYSIGLDIGTNSVGWAVITDEYKVPSKKFKVLGNTDRHSIKKNLIGALLFDSGET |
| | spCas9(H839A) | AEATRLKRTARRRYTRRKNRICYLQEIFSNEMAKVDDSFFHRLEESFLVEEDKKHER |
| | Nickase | HPIFGNIVDEVAYHEKYPTIYHLRKKLVDSTDKADLRLIYLALAHMIKFRGHFLIEGD |
| | | LNPDNSDVDKLFIQLVQTYNQLFEENPINASGVDAKAILSARLSKSRRLENLIAQLPG |
| | | EKKNGLFGNLIALSLGLTPNFKSNFDLAEDAKLQLSKDTYDDDLDNLLAQIGDQYA |
| | | DLFLAAKNLSDAILLSDILRVNTEITKAPLSASMIKRYDEHHQDLTLLKALVRQQLPE |
| | | KYKEIFFDQSKNGYAGYIDGGASQEEFYKFIKPILEKMDGTEELLVKLNREDLLRKQ |
| | | RTFDNGSIPHQIHLGELHAILRRQEDFYPFLKDNREKIEKILTFRIPYYVGPLARGNSR |
| | | FAWMTRKSEETITPWNFEEVVDKGASAQSFIERMTNFDKNLPNEKVLPKHSLLYEY |
| | | FTVYNELTKVKYVTEGMRKPAFLSGEQKKAIVDLLFKTNRKVTVKQLKEDYFKKIE |
| | | CFDSVEISGVEDRFNASLGTYHDLLKIIKDKDFLDNEENEDILEDIVLTLTLFEDREMI |
| | | EERLKTYAHLFDDKVMKQLKRRRYTGWGRLSRKLINGIRDKQSGKTILDFLKSDGF |
| | | ANRNFMQLIHDDSLTFKEDIQKAQVSGQGDSLHEHIANLAGSPAIKKGILQTVKVVD |
| | | ELVKVMGRHKPENIVIEMARENQTTQKGQKNSRERMKRIEEGIKELGSQILKEHPVE |
| | | NTQLQNEKLYLYYLQNGRDMYVDQELDINRLSDYDVDAIVPQSFLKDDSIDNKVLT |
| | | RSDKNRGKSDNVPSEEVVKKMKNYWRQLLNAKLITQRKFDNLTKAERGGLSELDK |
| | | AGFIKRQLVETRQITKHVAQILDSRMNTKYDENDKLIREVKVITLKSKLVSDFRKDF |
| | | QFYKVREINNYHHAHDAYLNAVVGTALIKKYPKLESEFVYGDYKVYDVRKMIAKS |
| | | EQEIGKATAKYFFYSNIMNFFKTEITLANGEIRKRPLIETNGETGEIVWDKGRDFATV |
| | | RKVLSMPQVNIVKKTEVQTGGFSKESILPKRNSDKLIARKKDWDPKKYGGFDSPTV |
| | | AYSVLVVAKVEKGKSKKLKSVKELLGITIMERSSFEKNPIDFLEAKGYKEVKKDLIIK |
| | | LPKYSLFELENGRKRMLASAGELQKGNELALPSKYVNFLYLASHYEKLKGSPEDNE |
| | | QKQLFVEQHKHYLDEIIEQISEFSKRVILADANLDKVLSAYNKHRDKPIREQAENIIH |
| | | LFTLTNLGAPAAFKYFDTTIDRKRYTSTKEVLDATLIHQSITGLYETRIDLSQLGGD |
| 3583 | WT sluCas9 | MNQKFILGLDIGITSVGYGLIDYETKNIIDAGVRLFPEANVENNEGRRSKRGSRRLKR |
| | (recognized NNGG | RRIHRLERVKKLLEDYNLLDQSQIPQSTNPYAIRVKGLSEALSKDELVIALLHIAKRR |
| | PAM) | GIHKIDVIDSNDDVGNELSTKEQLNKNSKLLKDKFVCQIQLERMNEGQVRGEKNRF |
| | | KTADIIKEIIQLLNVQKNFHQLDENFINKYIELVEMRREYFEGPGKGSPYGWEGDPKA |
| | | WYETLMGHCTYFPDELRSVKYAYSADLFNALNDLNNLVIQRDGLSKLEYHEKYHII |
| | | ENVFKQKKKPTLKQIANEINVNPEDIKGYRITKSGKPQFTEFKLYHDLKSVLFDQSIL |
| | | ENEDVLDQIAEILTIYQDKDSIKSKLTELDILLNEEDKENIAQLTGYTGTHRLSLKCIR |
| | | LVLEEQWYSSRNQMEIFTHLNIKPKKINLTAANKIPKAMIDEFILSPVVKRTFGQAIN |
| | | LINKIIEKYGVPEDIIIELARENNSKDKQKFINEMQKKNENTRKRINEIIGKYGNQNAK |
| | | RLVEKIRLHDEQEGKCLYSLESIPLEDLLNNPNHYEVDHIIPRSVSFDNSYHNKVLVK |
| | | QSENSKKSNLTPYQYFNSGKSKLSYNQFKQHILNLSKSQDRISKKKKEYLLEERDIN |
| | | KFEVQKEFINRNLVDTRYATRELTNYLKAYFSANNMNVKVKTINGSFTDYLRKVW |
| | | KFKKERNHGYKHHAEDALIIANADFLFKENKKLKAVNSVLEKPEIESKQLDIQVDSE |
| | | DNYSEMFIIPKQVQDIKDFRNFKYSHRVDKKPNRQLINDTLYSTRKKDNSTYIVQTIK |
| | | DIYAKDNTTLKKQFDKSPEKFLMYQHDPRTFEKLEVIMKQYANEKNPLAKYHEETG |
| | | EYLTKYSKKNNGPIVKSLKYIGNKLGSHLDVTHQFKSSTKKLVKLSIKPYRFDVYLT |
| | | DKGYKFITISYLDVLKKDNYYYIPEQKYDKLKLGKAIDKNAKFIASFYKNDLIKLDG |
| | | EIYKIIGVNSDTRNMIELDLPDIRYKEYCELNNIKGEPRIKKTIGKKVNSIEKLTTDVL |
| | | GNVFTNTQYTKPQLLFKRGN |
| 3584 | sluCas9(N582A) | MNQKFILGLDIGITSVGYGLIDYETKNIIDAGVRLFPEANVENNEGRRSKRGSRRLKR |
| | Nickase | RRIHRLERVKKLLEDYNLLDQSQIPQSTNPYAIRVKGLSEALSKDELVIALLHIAKRR |
| | | GIHKIDVIDSNDDVGNELSTKEQLNKNSKLLKDKFVCQIQLERMNEGQVRGEKNRF |
| | | KTADIIKEIIQLLNVQKNFHQLDENFINKYIELVEMRREYFEGPGKGSPYGWEGDPKA |
| | | WYETLMGHCTYFPDELRSVKYAYSADLFNALNDLNNLVIQRDGLSKLEYHEKYHII |
| | | ENVFKQKKKPTLKQIANEINVNPEDIKGYRITKSGKPQFTEFKLYHDLKSVLFDQSIL |
| | | ENEDVLDQIAEILTIYQDKDSIKSKLTELDILLNEEDKENIAQLTGYTGTHRLSLKCIR |
| | | LVLEEQWYSSRNQMEIFTHLNIKPKKINLTAANKIPKAMIDEFILSPVVKRTFGQAIN |
| | | LINKIIEKYGVPEDIIIELARENNSKDKQKFINEMQKKNENTRKRINEIIGKYGNQNAK |
| | | RLVEKIRLHDEQEGKCLYSLESIPLEDLLNNPNHYEVDHIIPRSVSFDNSYHNKVLVK |
| | | QSEASKKSNLTPYQYFNSGKSKLSYNQFKQHILNLSKSQDRISKKKKEYLLEERDIN |
| | | KFEVQKEFINRNLVDTRYATRELTNYLKAYFSANNMNVKVKTINGSFTDYLRKVW |
| | | KFKKERNHGYKHHAEDALIIANADFLFKENKKLKAVNSVLEKPEIESKQLDIQVDSE |
| | | DNYSEMFIIPKQVQDIKDFRNFKYSHRVDKKPNRQLINDTLYSTRKKDNSTYIVQTIK |
| | | DIYAKDNTTLKKQFDKSPEKFLMYQHDPRTFEKLEVIMKQYANEKNPLAKYHEETG |
| | | EYLTKYSKKNNGPIVKSLKYIGNKLGSHLDVTHQFKSSTKKLVKLSIKPYRFDVYLT |
| | | DKGYKFITISYLDVLKKDNYYYIPEQKYDKLKLGKAIDKNAKFIASFYKNDLIKLDG |
| | | EIYKIIGVNSDTRNMIELDLPDIRYKEYCELNNIKGEPRIKKTIGKKVNSIEKLTTDVL |
| | | GNVFTNTQYTKPQLLFKRGN |
| 3585 | Met(−) sluCas9 | NQKFILGLDIGITSVGYGLIDYETKNIIDAGVRLFPEANVENNEGRRSKRGSRRLKRR |
| | ( )Nickase | RIHRLERVKKLLEDYNLLDQSQIPQSTNPYAIRVKGLSEALSKDELVIALLHIAKRRGI |
| | | HKIDVIDSNDDVGNELSTKEQLNKNSKLLKDKFVCQIQLERMNEGQVRGEKNRFKT |
| | | ADIIKEIIQLLNVQKNFHQLDENFINKYIELVEMRREYFEGPGKGSPYGWEGDPKAW |
| | | YETLMGHCTYFPDELRSVKYAYSADLFNALNDLNNLVIQRDGLSKLEYHEKYHIIEN |
| | | VFKQKKKPTLKQIANEINVNPEDIKGYRITKSGKPQFTEFKLYHDLKSVLFDQSILEN |
| | | EDVLDQIAEILTIYQDKDSIKSKLTELDILLNEEDKENIAQLTGYTGTHRLSLKCIRLV |
| | | LEEQWYSSRNQMEIFTHLNIKPKKINLTAANKIPKAMIDEFILSPVVKRTFGQAINLIN |
| | | KIIEKYGVPEDIIIELARENNSKDKQKFINEMQKKNENTRKRINEIIGKYGNQNAKRL |
| | | VEKIRLHDEQEGKCLYSLESIPLEDLLNNPNHYEVDHIIPRSVSFDNSYHNKVLVKQS |
| | | EASKKSNLTPYQYFNSGKSKLSYNQFKQHILNLSKSQDRISKKKKEYLLEERDINKFE |
| | | VQKEFINRNLVDTRYATRELTNYLKAYFSANNMNVKVKTINGSFTDYLRKVWKFK |
| | | KERNHGYKHHAEDALIIANADFLFKENKKLKAVNSVLEKPEIESKQLDIQVDSEDNY |
| | | SEMFIIPKQVQDIKDFRNFKYSHRVDKKPNRQLINDTLYSTRKKDNSTYIVQTIKDIY |
| | | AKDNTTLKKQFDKSPEKFLMYQHDPRTFEKLEVIMKQYANEKNPLAKYHEETGEY |
| | | LTKYSKKNNGPIVKSLKYIGNKLGSHLDVTHQFKSSTKKLVKLSIKPYRFDVYLTDK |
| | | GYKFITISYLDVLKKDNYYYIPEQKYDKLKLGKAIDKNAKFIASFYKNDLIKLDGEIY |
| | | KIIGVNSDTRNMIELDLPDIRYKEYCELNNIKGEPRIKKTIGKKVNSIEKLTTDVLGNV |
| | | FTNTQYTKPQLLFKRGN |
| 3586 | Staphylococcus | MKRNYILGLDIGITSVGYGIIDYETRDVIDAGVRLFKEANVENNEGRRSKRGARRLK |
| | aureus Cas9 | RRRRHRIQRVKKLLFDYNLLTDHSELSGINPYEARVKGLSQKLSEEEFSAALLHLAK |
| | (saCas9) | RRGVHNVNEVEEDTGNELSTKEQISRNSKALEEKYVAELQLERLKKDGEVRGSINRF |
| | | KTSDYVKEAKQLLKVQKAYHQLDQSFIDTYIDLLETRRTYYEGPGEGSPFGWKDIK |
| | | EWYEMLMGHCTYFPEELRSVKYAYNADLYNALNDLNNLVITRDENEKLEYYEKFQ |
| | | IIENVFKQKKKPTLKQIAKEILVNEEDIKGYRVTSTGKPEFTNLKVYHDIKDITARKEII |
| | | ENAELLDQIAKILTIYQSSEDIQEELTNLNSELTQEEIEQISNLKGYTGTHNLSLKAINL |
| | | ILDELWHTNDNQIAIFNRLKLVPKKVDLSQQKEIPTTLVDDFILSPVVKRSFIQSIKVIN |
| | | AIIKKYGLPNDIIIELAREKNSKDAQKMINEMQKRNRQTNERIEEIIRTTGKENAKYLI |
| | | EKIKLHDMQEGKCLYSLEAIPLEDLLNNPFNYEVDHIIPRSVSFDNSFNNKVLVKQEE |
| | | NSKKGNRTPFQYLSSSDSKISYETFKKHILNLAKGKGRISKTKKEYLLEERDINRFSV |
| | | QKDFINRNLVDTRYATRGLMNLLRSYFRVNNLDVKVKSINGGFTSFLRRKWKFKKE |
| | | RNKGYKHHAEDALIIANADFIFKEWKKLDKAKKVMENQMFEEKQAESMPEIETEQE |
| | | YKEIFITPHQIKHIKDFKDYKYSHRVDKKPNRELINDTLYSTRKDDKGNTLIVNNLNG |
| | | LYDKDNDKLKKLINKSPEKLLMYHHDPQTYQKLKLIMEQYGDEKNPLYKYYEETG |
| | | NYLTKYSKKDNGPVIKKIKYYGNKLNAHLDITDDYPNSRNKVVKLSLKPYRFDVYL |
| | | DNGVYKFVTVKNLDVIKKENYYEVNSKCYEEAKKLKKISNQAEFIASFYNNDLIKIN |
| | | GELYRVIGVNNDLLNRIEVNMIDITYREYLENMNDKRPPRIIKTIASKTQSIKKYSTDI |
| | | LGNLYEVKSKKHPQIIKKG |
| 3587 | DO NOT | DO NOT INCLUDE and DELETE FROM SPECIFICATION |
| | INCLUDE- | |
| | replacement saCas9 | |
| | Nickase sequence | |
| | provided below. | |
| 3588 | saCas9 (N580A) | MKRNYILGLDIGITSVGYGIIDYETRDVIDAGVRLFKEANVENNEGRRSKRGARRLK |
| | Nickase | RRRRHRIQRVKKLLFDYNLLTDHSELSGINPYEARVKGLSQKLSEEEFSAALLHLAK |
| | | RRGVHNVNEVEEDTGNELSTKEQISRNSKALEEKYVAELQLERLKKDGEVRGSINRF |
| | | KTSDYVKEAKQLLKVQKAYHQLDQSFIDTYIDLLETRRTYYEGPGEGSPFGWKDIK |
| | | EWYEMLMGHCTYFPEELRSVKYAYNADLYNALNDLNNLVITRDENEKLEYYEKFQ |
| | | IIENVFKQKKKPTLKQIAKEILVNEEDIKGYRVTSTGKPEFTNLKVYHDIKDITARKEII |
| | | ENAELLDQIAKILTIYQSSEDIQEELTNLNSELTQEEIEQISNLKGYTGTHNLSLKAINL |
| | | ILDELWHTNDNQIAIFNRLKLVPKKVDLSQQKEIPTTLVDDFILSPVVKRSFIQSIKVIN |
| | | AIIKKYGLPNDIIIELAREKNSKDAQKMINEMQKRNRQTNERIEEIIRTTGKENAKYLI |
| | | EKIKLHDMQEGKCLYSLEAIPLEDLLNNPFNYEVDHIIPRSVSFDNSFNNKVLVKQEE |
| | | ASKKGNRTPFQYLSSSDSKISYETFKKHILNLAKGKGRISKTKKEYLLEERDINRFSV |
| | | QKDFINRNLVDTRYATRGLMNLLRSYFRVNNLDVKVKSINGGFTSFLRRKWKFKKE |
| | | RNKGYKHHAEDALIIANADFIFKEWKKLDKAKKVMENQMFEEKQAESMPEIETEQE |
| | | YKEIFITPHQIKHIKDFKDYKYSHRVDKKPNRELINDTLYSTRKDDKGNTLIVNNLNG |
| | | LYDKDNDKLKKLINKSPEKLLMYHHDPQTYQKLKLIMEQYGDEKNPLYKYYEETG |
| | | NYLTKYSKKDNGPVIKKIKYYGNKLNAHLDITDDYPNSRNKVVKLSLKPYRFDVYL |
| | | DNGVYKFVTVKNLDVIKKENYYEVNSKCYEEAKKLKKISNQAEFIASFYNNDLIKIN |
| | | GELYRVIGVNNDLLNRIEVNMIDITYREYLENMNDKRPPRIIKTIASKTQSIKKYSTDI |
| | | LGNLYEVKSKKHPQIIKKG |
| 3589 | Met(−) | KRNYILGLDIGITSVGYGIIDYETRDVIDAGVRLFKEANVENNEGRRSKRGARRLKR |
| | saCas9(N579A) | RRRHRIQRVKKLLFDYNLLTDHSELSGINPYEARVKGLSQKLSEEEFSAALLHLAKR |
| | Nickase | RGVHNVNEVEEDTGNELSTKEQISRNSKALEEKYVAELQLERLKKDGEVRGSINRF |
| | | KTSDYVKEAKQLLKVQKAYHQLDQSFIDTYIDLLETRRTYYEGPGEGSPFGWKDIK |
| | | EWYEMLMGHCTYFPEELRSVKYAYNADLYNALNDLNNLVITRDENEKLEYYEKFQ |
| | | IIENVFKQKKKPTLKQIAKEILVNEEDIKGYRVTSTGKPEFTNLKVYHDIKDITARKEII |
| | | ENAELLDQIAKILTIYQSSEDIQEELTNLNSELTQEEIEQISNLKGYTGTHNLSLKAINL |
| | | ILDELWHTNDNQIAIFNRLKLVPKKVDLSQQKEIPTTLVDDFILSPVVKRSFIQSIKVIN |
| | | AIIKKYGLPNDIIIELAREKNSKDAQKMINEMQKRNRQTNERIEEIIRTTGKENAKYLI |
| | | EKIKLHDMQEGKCLYSLEAIPLEDLLNNPFNYEVDHIIPRSVSFDNSFNNKVLVKQEE |
| | | ASKKGNRTPFQYLSSSDSKISYETFKKHILNLAKGKGRISKTKKEYLLEERDINRFSV |
| | | QKDFINRNLVDTRYATRGLMNLLRSYFRVNNLDVKVKSINGGFTSFLRRKWKFKKE |
| | | RNKGYKHHAEDALIIANADFIFKEWKKLDKAKKVMENQMFEEKQAESMPEIETEQE |
| | | YKEIFITPHQIKHIKDFKDYKYSHRVDKKPNRELINDTLYSTRKDDKGNTLIVNNLNG |
| | | LYDKDNDKLKKLINKSPEKLLMYHHDPQTYQKLKLIMEQYGDEKNPLYKYYEETG |
| | | NYLTKYSKKDNGPVIKKIKYYGNKLNAHLDITDDYPNSRNKVVKLSLKPYRFDVYL |
| | | DNGVYKFVTVKNLDVIKKENYYEVNSKCYEEAKKLKKISNQAEFIASFYNNDLIKIN |
| | | GELYRVIGVNNDLLNRIEVNMIDITYREYLENMNDKRPPRIIKTIASKTQSIKKYSTDI |
| | | LGNLYEVKSKKHPQIIKKG |
| 3590 | spCas9-NG | MDKKYSIGLDIGTNSVGWAVITDEYKVPSKKFKVLGNTDRHSIKKNLIGALLFDSGE |
| | | TAEATRLKRTARRRYTRRKNRICYLQEIFSNEMAKVDDSFFHRLEESFLVEEDKKHE |
| | | RHPIFGNIVDEVAYHEKYPTIYHLRKKLVDSTDKADLRLIYLALAHMIKFRGHFLIEG |
| | | DLNPDNSDVDKLFIQLVQTYNQLFEENPINASGVDAKAILSARLSKSRRLENLIAQLP |
| | | GEKKNGLFGNLIALSLGLTPNFKSNFDLAEDAKLQLSKDTYDDDLDNLLAQIGDQY |
| | | ADLFLAAKNLSDAILLSDILRVNTEITKAPLSASMIKRYDEHHQDLTLLKALVRQQLP |
| | | EKYKEIFFDQSKNGYAGYIDGGASQEEFYKFIKPILEKMDGTEELLVKLNREDLLRK |
| | | QRTFDNGSIPHQIHLGELHAILRRQEDFYPFLKDNREKIEKILTFRIPYYVGPLARGNS |
| | | RFAWMTRKSEETITPWNFEEVVDKGASAQSFIERMTNFDKNLPNEKVLPKHSLLYE |
| | | YFTVYNELTKVKYVTEGMRKPAFLSGEQKKAIVDLLFKTNRKVTVKQLKEDYFKKI |
| | | ECFDSVEISGVEDRFNASLGTYHDLLKIIKDKDFLDNEENEDILEDIVLTLTLFEDREM |
| | | IEERLKTYAHLFDDKVMKQLKRRRYTGWGRLSRKLINGIRDKQSGKTILDFLKSDGF |
| | | ANRNFMQLIHDDSLTFKEDIQKAQVSGQGDSLHEHIANLAGSPAIKKGILQTVKVVD |
| | | ELVKVMGRHKPENIVIEMARENQTTQKGQKNSRERMKRIEEGIKELGSQILKEHPVE |
| | | NTQLQNEKLYLYYLQNGRDMYVDQELDINRLSDYDVDHIVPQSFLKDDSIDNKVLT |
| | | RSDKNRGKSDNVPSEEVVKKMKNYWRQLLNAKLITQRKFDNLTKAERGGLSELDK |
| | | AGFIKRQLVETRQITKHVAQILDSRMNTKYDENDKLIREVKVITLKSKLVSDFRKDF |
| | | QFYKVREINNYHHAHDAYLNAVVGTALIKKYPKLESEFVYGDYKVYDVRKMIAKS |
| | | EQEIGKATAKYFFYSNIMNFFKTEITLANGEIRKRPLIETNGETGEIVWDKGRDFATV |
| | | RKVLSMPQVNIVKKTEVQTGGFSKESIRPKRNSDKLIARKKDWDPKKYGGFVSPTV |
| | | AYSVLVVAKVEKGKSKKLKSVKELLGITIMERSSFEKNPIDFLEAKGYKEVKKDLIIK |
| | | LPKYSLFELENGRKRMLASARFLQKGNELALPSKYVNFLYLASHYEKLKGSPEDNE |
| 3591 | spCas9-NG | QKQLFVEQHKHYLDEIIEQISEFSKRVILADANLDKVLSAYNKHRDKPIREQAENIIH |
| | (H840A) Nickase | LFTLTNLGAPRAFKYFDTTIDRKVYRSTKEVLDATLIHQSITGLYETRIDLSQLGGD |
| | | MDKKYSIGLDIGTNSVGWAVITDEYKVPSKKFKVLGNTDRHSIKKNLIGALLFDSGE |
| | | TAEATRLKRTARRRYTRRKNRICYLQEIFSNEMAKVDDSFFHRLEESFLVEEDKKHE |
| | | RHPIFGNIVDEVAYHEKYPTIYHLRKKLVDSTDKADLRLIYLALAHMIKFRGHFLIEG |
| | | DLNPDNSDVDKLFIQLVQTYNQLFEENPINASGVDAKAILSARLSKSRRLENLIAQLP |
| | | GEKKNGLFGNLIALSLGLTPNFKSNFDLAEDAKLQLSKDTYDDDLDNLLAQIGDQY |
| | | ADLFLAAKNLSDAILLSDILRVNTEITKAPLSASMIKRYDEHHQDLTLLKALVRQQLP |
| | | EKYKEIFFDQSKNGYAGYIDGGASQEEFYKFIKPILEKMDGTEELLVKLNREDLLRK |
| | | QRTFDNGSIPHQIHLGELHAILRRQEDFYPFLKDNREKIEKILTFRIPYYVGPLARGNS |
| | | RFAWMTRKSEETITPWNFEEVVDKGASAQSFIERMTNFDKNLPNEKVLPKHSLLYE |
| | | YFTVYNELTKVKYVTEGMRKPAFLSGEQKKAIVDLLFKTNRKVTVKQLKEDYFKKI |
| | | ECFDSVEISGVEDRFNASLGTYHDLLKIIKDKDFLDNEENEDILEDIVLTLTLFEDREM |
| | | IEERLKTYAHLFDDKVMKQLKRRRYTGWGRLSRKLINGIRDKQSGKTILDFLKSDGF |
| | | ANRNFMQLIHDDSLTFKEDIQKAQVSGQGDSLHEHIANLAGSPAIKKGILQTVKVVD |
| | | ELVKVMGRHKPENIVIEMARENQTTQKGQKNSRERMKRIEEGIKELGSQILKEHPVE |
| | | NTQLQNEKLYLYYLQNGRDMYVDQELDINRLSDYDVDAIVPQSFLKDDSIDNKVLT |
| | | RSDKNRGKSDNVPSEEVVKKMKNYWRQLLNAKLITQRKFDNLTKAERGGLSELDK |
| | | AGFIKRQLVETRQITKHVAQILDSRMNTKYDENDKLIREVKVITLKSKLVSDFRKDF |
| | | QFYKVREINNYHHAHDAYLNAVVGTALIKKYPKLESEFVYGDYKVYDVRKMIAKS |
| | | EQEIGKATAKYFFYSNIMNFFKTEITLANGEIRKRPLIETNGETGEIVWDKGRDFATV |
| 3592 | spCas9-NG | RKVLSMPQVNIVKKTEVQTGGFSKESIRPKRNSDKLIARKKDWDPKKYGGFVSPTV |
| | (H840A) Nickase | AYSVLVVAKVEKGKSKKLKSVKELLGITIMERSSFEKNPIDFLEAKGYKEVKKDLIIK |
| | | LPKYSLFELENGRKRMLASARFLQKGNELALPSKYVNFLYLASHYEKLKGSPEDNE |
| | | QKQLFVEQHKHYLDEIIEQISEFSKRVILADANLDKVLSAYNKHRDKPIREQAENIIH |
| | | LFTLTNLGAPRAFKYFDTTIDRKVYRSTKEVLDATLIHQSITGLYETRIDLSQLGGD |
| | | DKKYSIGLDIGTNSVGWAVITDEYKVPSKKFKVLGNTDRHSIKKNLIGALLFDSGET |
| | | AEATRLKRTARRRYTRRKNRICYLQEIFSNEMAKVDDSFFHRLEESFLVEEDKKHER |
| | | HPIFGNIVDEVAYHEKYPTIYHLRKKLVDSTDKADLRLIYLALAHMIKFRGHFLIEGD |
| | | LNPDNSDVDKLFIQLVQTYNQLFEENPINASGVDAKAILSARLSKSRRLENLIAQLPG |
| | | EKKNGLFGNLIALSLGLTPNFKSNFDLAEDAKLQLSKDTYDDDLDNLLAQIGDQYA |
| | | DLFLAAKNLSDAILLSDILRVNTEITKAPLSASMIKRYDEHHQDLTLLKALVRQQLPE |
| | | KYKEIFFDQSKNGYAGYIDGGASQEEFYKFIKPILEKMDGTEELLVKLNREDLLRKQ |
| | | RTFDNGSIPHQIHLGELHAILRRQEDFYPFLKDNREKIEKILTFRIPYYVGPLARGNSR |
| | | FAWMTRKSEETITPWNFEEVVDKGASAQSFIERMTNFDKNLPNEKVLPKHSLLYEY |
| | | FTVYNELTKVKYVTEGMRKPAFLSGEQKKAIVDLLFKTNRKVTVKQLKEDYFKKIE |
| | | CFDSVEISGVEDRFNASLGTYHDLLKIIKDKDFLDNEENEDILEDIVLTLTLFEDREMI |
| | | EERLKTYAHLFDDKVMKQLKRRRYTGWGRLSRKLINGIRDKQSGKTILDFLKSDGF |
| | | ANRNFMQLIHDDSLTFKEDIQKAQVSGQGDSLHEHIANLAGSPAIKKGILQTVKVVD |
| | | ELVKVMGRHKPENIVIEMARENQTTQKGQKNSRERMKRIEEGIKELGSQILKEHPVE |
| | | NTQLQNEKLYLYYLQNGRDMYVDQELDINRLSDYDVDAIVPQSFLKDDSIDNKVLT |
| | | RSDKNRGKSDNVPSEEVVKKMKNYWRQLLNAKLITQRKFDNLTKAERGGLSELDK |
| | | AGFIKRQLVETRQITKHVAQILDSRMNTKYDENDKLIREVKVITLKSKLVSDFRKDF |
| | | QFYKVREINNYHHAHDAYLNAVVGTALIKKYPKLESEFVYGDYKVYDVRKMIAKS |
| | | EQEIGKATAKYFFYSNIMNFFKTEITLANGEIRKRPLIETNGETGEIVWDKGRDFATV |
| 3593 | spCas9-VRQR | RKVLSMPQVNIVKKTEVQTGGFSKESIRPKRNSDKLIARKKDWDPKKYGGFVSPTV |
| | | AYSVLVVAKVEKGKSKKLKSVKELLGITIMERSSFEKNPIDFLEAKGYKEVKKDLIIK |
| | | LPKYSLFELENGRKRMLASARFLQKGNELALPSKYVNFLYLASHYEKLKGSPEDNE |
| | | QKQLFVEQHKHYLDEIIEQISEFSKRVILADANLDKVLSAYNKHRDKPIREQAENIIH |
| | | LFTLTNLGAPRAFKYFDTTIDRKVYRSTKEVLDATLIHQSITGLYETRIDLSQLGGD |
| | | MDKKYSIGLDIGTNSVGWAVITDEYKVPSKKFKVLGNTDRHSIKKNLIGALLFDSGE |
| | | TAEATRLKRTARRRYTRRKNRICYLQEIFSNEMAKVDDSFFHRLEESFLVEEDKKHE |
| | | RHPIFGNIVDEVAYHEKYPTIYHLRKKLVDSTDKADLRLIYLALAHMIKFRGHFLIEG |
| | | DLNPDNSDVDKLFIQLVQTYNQLFEENPINASGVDAKAILSARLSKSRRLENLIAQLP |
| | | GEKKNGLFGNLIALSLGLTPNFKSNFDLAEDAKLQLSKDTYDDDLDNLLAQIGDQY |
| | | ADLFLAAKNLSDAILLSDILRVNTEITKAPLSASMIKRYDEHHQDLTLLKALVRQQLP |
| | | EKYKEIFFDQSKNGYAGYIDGGASQEEFYKFIKPILEKMDGTEELLVKLNREDLLRK |
| | | QRTFDNGSIPHQIHLGELHAILRRQEDFYPFLKDNREKIEKILTFRIPYYVGPLARGNS |
| | | RFAWMTRKSEETITPWNFEEVVDKGASAQSFIERMTNFDKNLPNEKVLPKHSLLYE |
| | | YFTVYNELTKVKYVTEGMRKPAFLSGEQKKAIVDLLFKTNRKVTVKQLKEDYFKKI |
| | | ECFDSVEISGVEDRFNASLGTYHDLLKIIKDKDFLDNEENEDILEDIVLTLTLFEDREM |
| | | IEERLKTYAHLFDDKVMKQLKRRRYTGWGRLSRKLINGIRDKQSGKTILDFLKSDGF |
| | | ANRNFMQLIHDDSLTFKEDIQKAQVSGQGDSLHEHIANLAGSPAIKKGILQTVKVVD |
| | | ELVKVMGRHKPENIVIEMARENQTTQKGQKNSRERMKRIEEGIKELGSQILKEHPVE |
| | | NTQLQNEKLYLYYLQNGRDMYVDQELDINRLSDYDVDHIVPQSFLKDDSIDNKVLT |
| | | RSDKNRGKSDNVPSEEVVKKMKNYWRQLLNAKLITQRKFDNLTKAERGGLSELDK |
| | | AGFIKRQLVETRQITKHVAQILDSRMNTKYDENDKLIREVKVITLKSKLVSDFRKDF |
| | | QFYKVREINNYHHAHDAYLNAVVGTALIKKYPKLESEFVYGDYKVYDVRKMIAKS |
| | | EQEIGKATAKYFFYSNIMNFFKTEITLANGEIRKRPLIETNGETGEIVWDKGRDFATV |
| 3594 | spCas9-VRQR | RKVLSMPQVNIVKKTEVQTGGFSKESILPKRNSDKLIARKKDWDPKKYGGFVSPTV |
| | (H840A) Nickase | AYSVLVVAKVEKGKSKKLKSVKELLGITIMERSSFEKNPIDFLEAKGYKEVKKDLIIK |
| | | LPKYSLFELENGRKRMLASARELQKGNELALPSKYVNFLYLASHYEKLKGSPEDNE |
| | | QKQLFVEQHKHYLDEIIEQISEFSKRVILADANLDKVLSAYNKHRDKPIREQAENIIH |
| | | LFTLTNLGAPAAFKYFDTTIDRKQYRSTKEVLDATLIHQSITGLYETRIDLSQLGGD |
| | | MDKKYSIGLDIGTNSVGWAVITDEYKVPSKKFKVLGNTDRHSIKKNLIGALLFDSGE |
| | | TAEATRLKRTARRRYTRRKNRICYLQEIFSNEMAKVDDSFFHRLEESFLVEEDKKHE |
| | | RHPIFGNIVDEVAYHEKYPTIYHLRKKLVDSTDKADLRLIYLALAHMIKFRGHFLIEG |
| | | DLNPDNSDVDKLFIQLVQTYNQLFEENPINASGVDAKAILSARLSKSRRLENLIAQLP |
| | | GEKKNGLFGNLIALSLGLTPNFKSNFDLAEDAKLQLSKDTYDDDLDNLLAQIGDQY |
| | | ADLFLAAKNLSDAILLSDILRVNTEITKAPLSASMIKRYDEHHQDLTLLKALVRQQLP |
| | | EKYKEIFFDQSKNGYAGYIDGGASQEEFYKFIKPILEKMDGTEELLVKLNREDLLRK |
| | | QRTFDNGSIPHQIHLGELHAILRRQEDFYPFLKDNREKIEKILTFRIPYYVGPLARGNS |
| | | RFAWMTRKSEETITPWNFEEVVDKGASAQSFIERMTNFDKNLPNEKVLPKHSLLYE |
| | | YFTVYNELTKVKYVTEGMRKPAFLSGEQKKAIVDLLFKTNRKVTVKQLKEDYFKKI |
| | | ECFDSVEISGVEDRFNASLGTYHDLLKIIKDKDFLDNEENEDILEDIVLTLTLFEDREM |
| | | IEERLKTYAHLFDDKVMKQLKRRRYTGWGRLSRKLINGIRDKQSGKTILDFLKSDGF |
| | | ANRNFMQLIHDDSLTFKEDIQKAQVSGQGDSLHEHIANLAGSPAIKKGILQTVKVVD |
| | | ELVKVMGRHKPENIVIEMARENQTTQKGQKNSRERMKRIEEGIKELGSQILKEHPVE |
| | | NTQLQNEKLYLYYLQNGRDMYVDQELDINRLSDYDVDAIVPQSFLKDDSIDNKVLT |
| | | RSDKNRGKSDNVPSEEVVKKMKNYWRQLLNAKLITQRKFDNLTKAERGGLSELDK |
| | | AGFIKRQLVETRQITKHVAQILDSRMNTKYDENDKLIREVKVITLKSKLVSDFRKDF |
| | | QFYKVREINNYHHAHDAYLNAVVGTALIKKYPKLESEFVYGDYKVYDVRKMIAKS |
| | | EQEIGKATAKYFFYSNIMNFFKTEITLANGEIRKRPLIETNGETGEIVWDKGRDFATV |
| 3595 | Met(−) spCas9- | RKVLSMPQVNIVKKTEVQTGGFSKESILPKRNSDKLIARKKDWDPKKYGGFVSPTV |
| | VRQR (H839A) | AYSVLVVAKVEKGKSKKLKSVKELLGITIMERSSFEKNPIDFLEAKGYKEVKKDLIIK |
| | Nickase | LPKYSLFELENGRKRMLASARELQKGNELALPSKYVNFLYLASHYEKLKGSPEDNE |
| | | QKQLFVEQHKHYLDEIIEQISEFSKRVILADANLDKVLSAYNKHRDKPIREQAENIIH |
| | | LFTLTNLGAPAAFKYFDTTIDRKQYRSTKEVLDATLIHQSITGLYETRIDLSQLGGD |
| | | DKKYSIGLDIGTNSVGWAVITDEYKVPSKKFKVLGNTDRHSIKKNLIGALLFDSGET |
| | | AEATRLKRTARRRYTRRKNRICYLQEIFSNEMAKVDDSFFHRLEESFLVEEDKKHER |
| | | HPIFGNIVDEVAYHEKYPTIYHLRKKLVDSTDKADLRLIYLALAHMIKFRGHFLIEGD |
| | | LNPDNSDVDKLFIQLVQTYNQLFEENPINASGVDAKAILSARLSKSRRLENLIAQLPG |
| | | EKKNGLFGNLIALSLGLTPNFKSNFDLAEDAKLQLSKDTYDDDLDNLLAQIGDQYA |
| | | DLFLAAKNLSDAILLSDILRVNTEITKAPLSASMIKRYDEHHQDLTLLKALVRQQLPE |
| | | KYKEIFFDQSKNGYAGYIDGGASQEEFYKFIKPILEKMDGTEELLVKLNREDLLRKQ |
| | | RTFDNGSIPHQIHLGELHAILRRQEDFYPFLKDNREKIEKILTFRIPYYVGPLARGNSR |
| | | FAWMTRKSEETITPWNFEEVVDKGASAQSFIERMTNFDKNLPNEKVLPKHSLLYEY |
| | | FTVYNELTKVKYVTEGMRKPAFLSGEQKKAIVDLLFKTNRKVTVKQLKEDYFKKIE |
| | | CFDSVEISGVEDRFNASLGTYHDLLKIIKDKDFLDNEENEDILEDIVLTLTLFEDREMI |
| | | EERLKTYAHLFDDKVMKQLKRRRYTGWGRLSRKLINGIRDKQSGKTILDFLKSDGF |
| | | ANRNFMQLIHDDSLTFKEDIQKAQVSGQGDSLHEHIANLAGSPAIKKGILQTVKVVD |
| | | ELVKVMGRHKPENIVIEMARENQTTQKGQKNSRERMKRIEEGIKELGSQILKEHPVE |
| | | NTQLQNEKLYLYYLQNGRDMYVDQELDINRLSDYDVDAIVPQSFLKDDSIDNKVLT |
| | | RSDKNRGKSDNVPSEEVVKKMKNYWRQLLNAKLITQRKFDNLTKAERGGLSELDK |
| | | AGFIKRQLVETRQITKHVAQILDSRMNTKYDENDKLIREVKVITLKSKLVSDFRKDF |
| | | QFYKVREINNYHHAHDAYLNAVVGTALIKKYPKLESEFVYGDYKVYDVRKMIAKS |
| | | EQEIGKATAKYFFYSNIMNFFKTEITLANGEIRKRPLIETNGETGEIVWDKGRDFATV |
| 3596 | SpRY | RKVLSMPQVNIVKKTEVQTGGFSKESILPKRNSDKLIARKKDWDPKKYGGFVSPTV |
| | | AYSVLVVAKVEKGKSKKLKSVKELLGITIMERSSFEKNPIDFLEAKGYKEVKKDLIIK |
| | | LPKYSLFELENGRKRMLASARELQKGNELALPSKYVNFLYLASHYEKLKGSPEDNE |
| | | QKQLFVEQHKHYLDEIIEQISEFSKRVILADANLDKVLSAYNKHRDKPIREQAENIIH |
| | | LFTLTNLGAPAAFKYFDTTIDRKQYRSTKEVLDATLIHQSITGLYETRIDLSQLGGD |
| | | MDKKYSIGLDIGTNSVGWAVITDEYKVPSKKFKVLGNTDRHSIKKNLIGALLFDSGE |
| | | TAERTRLKRTARRRYTRRKNRICYLQEIFSNEMAKVDDSFFHRLEESFL VEEDKKHE |
| | | RHPIFGNIVDEVAYHEKYPTIYHLRKKLVDSTDKADLRLIYLALAHMIKFRGHFLIEG |
| | | DLNPDNSDVDKLFIQLVQTYNQLFEENPINASGVDAKAILSARLSKSRRLENLIAQLP |
| | | GEKKNGLFGNLIALSLGLTPNFKSNFDLAEDAKLQLSKDTYDDDLDNLLAQIGDQY |
| | | ADLFLAAKNLSDAILLSDILRVNTEITKAPLSASMIKRYDEHHQDLTLLKALVRQQLP |
| | | EKYKEIFFDQSKNGYAGYIDGGASQEEFYKFIKPILEKMDGTEELLVKLNREDLLRK |
| | | QRTFDNGSIPHQIHLGELHAILRRQEDFYPFLKDNREKIEKILTFRIPYYVGPLARGNS |
| | | RFAWMTRKSEETITPWNFEEVVDKGASAQSFIERMTNFDKNLPNEKVLPKHSLLYE |
| | | YFTVYNELTKVKYVTEGMRKPAFLSGEQKKAIVDLLFKTNRKVTVKQLKEDYFKKI |
| | | ECFDSVEISGVEDRFNASLGTYHDLLKIIKDKDFLDNEENEDILEDIVLTLTLFEDREM |
| | | IEERLKTYAHLFDDKVMKQLKRRRYTGWGRLSRKLINGIRDKQSGKTILDFLKSDGF |
| | | ANRNFMQLIHDDSLTFKEDIQKAQVSGQGDSLHEHIANLAGSPAIKKGILQTVKVVD |
| | | ELVKVMGRHKPENIVIEMARENQTTQKGQKNSRERMKRIEEGIKELGSQILKEHPVE |
| | | NTQLQNEKLYLYYLQNGRDMYVDQELDINRLSDYDVDHIVPQSFLKDDSIDNKVLT |
| | | RSDKNRGKSDNVPSEEVVKKMKNYWRQLLNAKLITQRKFDNLTKAERGGLSELDK |
| | | AGFIKRQLVETRQITKHVAQILDSRMNTKYDENDKLIREVKVITLKSKLVSDFRKDF |
| | | QFYKVREINNYHHAHDAYLNAVVGTALIKKYPKLESEFVYGDYKVYDVRKMIAKS |
| | | EQEIGKATAKYFFYSNIMNFFKTEITLANGEIRKRPLIETNGETGEIVWDKGRDFATV |
| 3597 | SpRY(H840A) | RKVLSMPQVNIVKKTEVQTGGFSKESIRPKRNSDKLIARKKDWDPKKYGGFLWPTV |
| | Nickase | AYSVLVVAKVEKGKSKKLKSVKELLGITIMERSSFEKNPIDFLEAKGYKEVKKDLIIK |
| | | LPKYSLFELENGRKRMLASAKQLQKGNELALPSKYVNFLYLASHYEKLKGSPEDNE |
| | | QKQLFVEQHKHYLDEIIEQISEFSKRVILADANLDKVLSAYNKHRDKPIREQAENIIH |
| | | LFTLTRLGAPRAFKYFDTTIDPKQYRSTKEVLDATLIHQSITGLYETRIDLSQLGGD |
| | | MDKKYSIGLDIGTNSVGWAVITDEYKVPSKKFKVLGNTDRHSIKKNLIGALLFDSGE |
| | | TAERTRLKRTARRRYTRRKNRICYLQEIFSNEMAKVDDSFFHRLEESFL VEEDKKHE |
| | | RHPIFGNIVDEVAYHEKYPTIYHLRKKLVDSTDKADLRLIYLALAHMIKFRGHFLIEG |
| | | DLNPDNSDVDKLFIQLVQTYNQLFEENPINASGVDAKAILSARLSKSRRLENLIAQLP |
| | | GEKKNGLFGNLIALSLGLTPNFKSNFDLAEDAKLQLSKDTYDDDLDNLLAQIGDQY |
| | | ADLFLAAKNLSDAILLSDILRVNTEITKAPLSASMIKRYDEHHQDLTLLKALVRQQLP |
| | | EKYKEIFFDQSKNGYAGYIDGGASQEEFYKFIKPILEKMDGTEELLVKLNREDLLRK |
| | | QRTFDNGSIPHQIHLGELHAILRRQEDFYPFLKDNREKIEKILTFRIPYYVGPLARGNS |
| | | RFAWMTRKSEETITPWNFEEVVDKGASAQSFIERMTNFDKNLPNEKVLPKHSLLYE |
| | | YFTVYNELTKVKYVTEGMRKPAFLSGEQKKAIVDLLFKTNRKVTVKQLKEDYFKKI |
| | | ECFDSVEISGVEDRFNASLGTYHDLLKIIKDKDFLDNEENEDILEDIVLTLTLFEDREM |
| | | IEERLKTYAHLFDDKVMKQLKRRRYTGWGRLSRKLINGIRDKQSGKTILDFLKSDGF |
| | | ANRNFMQLIHDDSLTFKEDIQKAQVSGQGDSLHEHIANLAGSPAIKKGILQTVKVVD |
| | | ELVKVMGRHKPENIVIEMARENQTTQKGQKNSRERMKRIEEGIKELGSQILKEHPVE |
| | | NTQLQNEKLYLYYLQNGRDMYVDQELDINRLSDYDVDAIVPQSFLKDDSIDNKVLT |
| | | RSDKNRGKSDNVPSEEVVKKMKNYWRQLLNAKLITQRKFDNLTKAERGGLSELDK |
| | | AGFIKRQLVETRQITKHVAQILDSRMNTKYDENDKLIREVKVITLKSKLVSDFRKDF |
| | | QFYKVREINNYHHAHDAYLNAVVGTALIKKYPKLESEFVYGDYKVYDVRKMIAKS |
| | | EQEIGKATAKYFFYSNIMNFFKTEITLANGEIRKRPLIETNGETGEIVWDKGRDFATV |
| 3598 | Met(−) | RKVLSMPQVNIVKKTEVQTGGFSKESIRPKRNSDKLIARKKDWDPKKYGGFLWPTV |
| | SpRY(H839A) | AYSVLVVAKVEKGKSKKLKSVKELLGITIMERSSFEKNPIDFLEAKGYKEVKKDLIIK |
| | Nickase | LPKYSLFELENGRKRMLASAKQLQKGNELALPSKYVNFLYLASHYEKLKGSPEDNE |
| | | QKQLFVEQHKHYLDEIIEQISEFSKRVILADANLDKVLSAYNKHRDKPIREQAENIIH |
| | | LFTLTRLGAPRAFKYFDTTIDPKQYRSTKEVLDATLIHQSITGLYETRIDLSQLGGD |
| | | DKKYSIGLDIGTNSVGWAVITDEYKVPSKKFKVLGNTDRHSIKKNLIGALLFDSGET |
| | | AERTRLKRTARRRYTRRKNRICYLQEIFSNEMAKVDDSFFHRLEESFLVEEDKKHER |
| | | HPIFGNIVDEVAYHEKYPTIYHLRKKLVDSTDKADLRLIYLALAHMIKFRGHFLIEGD |
| | | LNPDNSDVDKLFIQLVQTYNQLFEENPINASGVDAKAILSARLSKSRRLENLIAQLPG |
| | | EKKNGLFGNLIALSLGLTPNFKSNFDLAEDAKLQLSKDTYDDDLDNLLAQIGDQYA |
| | | DLFLAAKNLSDAILLSDILRVNTEITKAPLSASMIKRYDEHHQDLTLLKALVRQQLPE |
| | | KYKEIFFDQSKNGYAGYIDGGASQEEFYKFIKPILEKMDGTEELLVKLNREDLLRKQ |
| | | RTFDNGSIPHQIHLGELHAILRRQEDFYPFLKDNREKIEKILTFRIPYYVGPLARGNSR |
| | | FAWMTRKSEETITPWNFEEVVDKGASAQSFIERMTNFDKNLPNEKVLPKHSLLYEY |
| | | FTVYNELTKVKYVTEGMRKPAFLSGEQKKAIVDLLFKTNRKVTVKQLKEDYFKKIE |
| | | CFDSVEISGVEDRFNASLGTYHDLLKIIKDKDFLDNEENEDILEDIVLTLTLFEDREMI |
| | | EERLKTYAHLFDDKVMKQLKRRRYTGWGRLSRKLINGIRDKQSGKTILDFLKSDGF |
| | | ANRNFMQLIHDDSLTFKEDIQKAQVSGQGDSLHEHIANLAGSPAIKKGILQTVKVVD |
| | | ELVKVMGRHKPENIVIEMARENQTTQKGQKNSRERMKRIEEGIKELGSQILKEHPVE |
| | | NTQLQNEKLYLYYLQNGRDMYVDQELDINRLSDYDVDAIVPQSFLKDDSIDNKVLT |
| | | RSDKNRGKSDNVPSEEVVKKMKNYWRQLLNAKLITQRKFDNLTKAERGGLSELDK |
| | | AGFIKRQLVETRQITKHVAQILDSRMNTKYDENDKLIREVKVITLKSKLVSDFRKDF |
| | | QFYKVREINNYHHAHDAYLNAVVGTALIKKYPKLESEFVYGDYKVYDVRKMIAKS |
| | | EQEIGKATAKYFFYSNIMNFFKTEITLANGEIRKRPLIETNGETGEIVWDKGRDFATV |
| 3599 | SRGN3.1 | RKVLSMPQVNIVKKTEVQTGGFSKESIRPKRNSDKLIARKKDWDPKKYGGFLWPTV |
| | | AYSVLVVAKVEKGKSKKLKSVKELLGITIMERSSFEKNPIDFLEAKGYKEVKKDLIIK |
| | | LPKYSLFELENGRKRMLASAKQLQKGNELALPSKYVNFLYLASHYEKLKGSPEDNE |
| | | QKQLFVEQHKHYLDEIIEQISEFSKRVILADANLDKVLSAYNKHRDKPIREQAENIIH |
| | | LFTLTRLGAPRAFKYFDTTIDPKQYRSTKEVLDATLIHQSITGLYETRIDLSQLGGD |
| | | MNQKFILGLDIGITSVGYGLIDYETKNIIDAGVRLFPEANVENNEGRRSKRGSRRLKR |
| | | RRIHRLERVKLLLTEYDLINKEQIPTSNNPYQIRVKGLSEILSKDELAIALLHLAKRRG |
| | | IHNVDVAADKEETASDSLSTKDQINKNAKFLESRYVCELQKERLENEGHVRGVENR |
| | | FLTKDIVREAKKIIDTQMQYYPEIDETFKEKYISLVETRREYFEGPGQGSPFGWNGDL |
| | | KKWYEMLMGHCTYFPQELRSVKYAYSADLFNALNDLNNLIIQRDNSEKLEYHEKY |
| | | HIIENVFKQKKKPTLKQIAKEIGVNPEDIKGYRITKSGTPEFTSFKLFHDLKKVVKDH |
| | | AILDDIDLLNQIAEILTIYQDKDSIVAELGQLEYLMSEADKQSISELTGYTGTHSLSLK |
| | | CMNMIIDELWHSSMNQMEVFTYLNMRPKKYELKGYQRIPTDMIDDAILSPVVKRTF |
| | | IQSINVINKVIEKYGIPEDIIIELARENNSDDRKKFINNLQKKNEATRKRINEIIGQTGN |
| | | QNAKRIVEKIRLHDQQEGKCLYSLESIPLEDLLNNPNHYEVDHIIPRSVSFDNSYHNK |
| | | VLVKQSENSKKSNLTPYQYFNSGKSKLSYNQFKQHILNLSKSQDRISKKKKEYLLEE |
| | | RDINKFEVQKEFINRNLVDTRYATRELTNYLKAYFSANNMNVKVKTINGSFTDYLR |
| | | KVWKFKKERNHGYKHHAEDALIIANADFLFKENKKLKAVNSVLEKPEIETKQLDIQ |
| | | VDSEDNYSEMFIIPKQVQDIKDFRNFKYSHRVDKKPNRQLINDTLYSTRKKDNSTYI |
| | | VQTIKDIYAKDNTTLKKQFDKSPEKFLMYQHDPRTFEKLEVIMKQYANEKNPLAKY |
| | | HEETGEYLTKYSKKNNGPIVKSLKYIGNKLGSHLDVTHQFKSSTKKLVKLSIKNYRF |
| | | DVYLTEKGYKFVTIAYLNVFKKDNYYYIPKDKYQELKEKKKIKDTDQFIASFYKND |
| | | LIKLNGDLYKIIGVNSDDRNIIELDYYDIKYKDYCEINNIKGEPRIKKTIGKKTESIEKF |
| | | TTDVLGNLYLHSTEKAPQLIFKRGL |
| 3600 | sRGN3.1(N585A) | MNQKFILGLDIGITSVGYGLIDYETKNIIDAGVRLFPEANVENNEGRRSKRGSRRLKR |
| | Nickase | RRIHRLERVKLLLTEYDLINKEQIPTSNNPYQIRVKGLSEILSKDELAIALLHLAKRRG |
| | | IHNVDVAADKEETASDSLSTKDQINKNAKFLESRYVCELQKERLENEGHVRGVENR |
| | | FLTKDIVREAKKIIDTQMQYYPEIDETFKEKYISLVETRREYFEGPGQGSPFGWNGDL |
| | | KKWYEMLMGHCTYFPQELRSVKYAYSADLFNALNDLNNLIIQRDNSEKLEYHEKY |
| | | HIIENVFKQKKKPTLKQIAKEIGVNPEDIKGYRITKSGTPEFTSFKLFHDLKKVVKDH |
| | | AILDDIDLLNQIAEILTIYQDKDSIVAELGQLEYLMSEADKQSISELTGYTGTHSLSLK |
| | | CMNMIIDELWHSSMNQMEVFTYLNMRPKKYELKGYQRIPTDMIDDAILSPVVKRTF |
| | | IQSINVINKVIEKYGIPEDIIIELARENNSDDRKKFINNLQKKNEATRKRINEIIGQTGN |
| | | QNAKRIVEKIRLHDQQEGKCLYSLESIPLEDLLNNPNHYEVDHIIPRSVSFDNSYHNK |
| | | VLVKQSEASKKSNLTPYQYFNSGKSKLSYNQFKQHILNLSKSQDRISKKKKEYLLEE |
| | | RDINKFEVQKEFINRNLVDTRYATRELTNYLKAYFSANNMNVKVKTINGSFTDYLR |
| | | KVWKFKKERNHGYKHHAEDALIIANADFLFKENKKLKAVNSVLEKPEIETKQLDIQ |
| | | VDSEDNYSEMFIIPKQVQDIKDFRNFKYSHRVDKKPNRQLINDTLYSTRKKDNSTYI |
| | | VQTIKDIYAKDNTTLKKQFDKSPEKFLMYQHDPRTFEKLEVIMKQYANEKNPLAKY |
| | | HEETGEYLTKYSKKNNGPIVKSLKYIGNKLGSHLDVTHQFKSSTKKLVKLSIKNYRF |
| | | DVYLTEKGYKFVTIAYLNVFKKDNYYYIPKDKYQELKEKKKIKDTDQFIASFYKND |
| | | LIKLNGDLYKIIGVNSDDRNIIELDYYDIKYKDYCEINNIKGEPRIKKTIGKKTESIEKF |
| | | TTDVLGNLYLHSTEKAPQLIFKRGL |
| 3601 | Met(−) | NQKFILGLDIGITSVGYGLIDYETKNIIDAGVRLFPEANVENNEGRRSKRGSRRLKRR |
| | SRGN3.1(N584A)N | RIHRLERVKLLLTEYDLINKEQIPTSNNPYQIRVKGLSEILSKDELAIALLHLAKRRGI |
| | ickase | HNVDVAADKEETASDSLSTKDQINKNAKFLESRYVCELQKERLENEGHVRGVENRF |
| | | LTKDIVREAKKIIDTQMQYYPEIDETFKEKYISLVETRREYFEGPGQGSPFGWNGDLK |
| | | KWYEMLMGHCTYFPQELRSVKYAYSADLFNALNDLNNLIIQRDNSEKLEYHEKYHI |
| | | IENVFKQKKKPTLKQIAKEIGVNPEDIKGYRITKSGTPEFTSFKLFHDLKKVVKDHAI |
| | | LDDIDLLNQIAEILTIYQDKDSIVAELGQLEYLMSEADKQSISELTGYTGTHSLSLKC |
| | | MNMIIDELWHSSMNQMEVFTYLNMRPKKYELKGYQRIPTDMIDDAILSPVVKRTFI |
| | | QSINVINKVIEKYGIPEDIIIELARENNSDDRKKFINNLQKKNEATRKRINEIIGQTGNQ |
| | | NAKRIVEKIRLHDQQEGKCLYSLESIPLEDLLNNPNHYEVDHIIPRSVSFDNSYHNKV |
| | | LVKQSEASKKSNLTPYQYFNSGKSKLSYNQFKQHILNLSKSQDRISKKKKEYLLEER |
| | | DINKFEVQKEFINRNLVDTRYATRELTNYLKAYFSANNMNVKVKTINGSFTDYLRK |
| | | VWKFKKERNHGYKHHAEDALIIANADFLFKENKKLKAVNSVLEKPEIETKQLDIQV |
| | | DSEDNYSEMFIIPKQVQDIKDFRNFKYSHRVDKKPNRQLINDTLYSTRKKDNSTYIV |
| | | QTIKDIYAKDNTTLKKQFDKSPEKFLMYQHDPRTFEKLEVIMKQYANEKNPLAKYH |
| | | EETGEYLTKYSKKNNGPIVKSLKYIGNKLGSHLDVTHQFKSSTKKLVKLSIKNYRFD |
| | | VYLTEKGYKFVTIAYLNVFKKDNYYYIPKDKYQELKEKKKIKDTDQFIASFYKNDLI |
| | | KLNGDLYKIIGVNSDDRNIIELDYYDIKYKDYCEINNIKGEPRIKKTIGKKTESIEKFTT |
| | | DVLGNLYLHSTEKAPQLIFKRGL |
| 3602 | SRGN3.3 | MNQKFILGLDIGITSVGYGLIDYETKNIIDAGVRLFPEANVENNEGRRSKRGSRRLKR |
| | | RRIHRLERVKLLLTEYDLINKEQIPTSNNPYQIRVKGLSEILSKDELAIALLHLAKRRG |
| | | IHNVDVAADKEETASDSLSTKDQINKNAKFLESRYVCELQKERLENEGHVRGVENR |
| | | FLTKDIVREAKKIIDTQMQYYPEIDETFKEKYISLVETRREYFEGPGQGSPFGWNGDL |
| | | KKWYEMLMGHCTYFPQELRSVKYAYSADLFNALNDLNNLIIQRDNSEKLEYHEKY |
| | | HIIENVFKQKKKPTLKQIAKEIGVNPEDIKGYRITKSGTPEFTSFKLFHDLKKVVKDH |
| | | AILDDIDLLNQIAEILTIYQDKDSIVAELGQLEYLMSEADKQSISELTGYTGTHSLSLK |
| | | CMNMIIDELWHSSMNQMEVFTYLNMRPKKYELKGYQRIPTDMIDDAILSPVVKRTF |
| | | IQSINVINKVIEKYGIPEDIIIELARENNSDDRKKFINNLQKKNEATRKRINEIIGQTGN |
| | | QNAKRIVEKIRLHDQQEGKCLYSLESIPLEDLLNNPNHYEVDHIIPRSVSFDNSYHNK |
| | | VLVKQSENSKKSNLTPYQYFNSGKSKLSYNQFKQHILNLSKSQDRISKKKKEYLLEE |
| | | RDINKFEVQKEFINRNLVDTRYATRELTSYLKAYFSANNMDVKVKTINGSFTNHLR |
| | | KVWRFDKYRNHGYKHHAEDALIIANADFLFKENKKLQNTNKILEKPTIENNTKKVT |
| | | VEKEEDYNNVFETPKLVEDIKQYRDYKFSHRVDKKPNRQLINDTLYSTRMKDEHDY |
| | | IVQTITDIYGKDNTNLKKQFNKNPEKFLMYQNDPKTFEKLSIIMKQYSDEKNPLAKY |
| | | YEETGEYLTKYSKKNNGPIVKKIKLLGNKVGNHLDVTNKYENSTKKLVKLSIKNYR |
| | | FDVYLTEKGYKFVTIAYLNVFKKDNYYYIPKDKYQELKEKKKIKDTDQFIASFYKN |
| | | DLIKLNGDLYKIIGVNSDDRNIIELDYYDIKYKDYCEINNIKGEPRIKKTIGKKTESIEK |
| | | FTTDVLGNLYLHSTEKAPQLIFKRGL |
| 3603 | SRGN3.3(N585A) | MNQKFILGLDIGITSVGYGLIDYETKNIIDAGVRLFPEANVENNEGRRSKRGSRRLKR |
| | Nickase | RRIHRLERVKLLLTEYDLINKEQIPTSNNPYQIRVKGLSEILSKDELAIALLHLAKRRG |
| | | IHNVDVAADKEETASDSLSTKDQINKNAKFLESRYVCELQKERLENEGHVRGVENR |
| | | FLTKDIVREAKKIIDTQMQYYPEIDETFKEKYISLVETRREYFEGPGQGSPFGWNGDL |
| | | KKWYEMLMGHCTYFPQELRSVKYAYSADLFNALNDLNNLIIQRDNSEKLEYHEKY |
| | | HIIENVFKQKKKPTLKQIAKEIGVNPEDIKGYRITKSGTPEFTSFKLFHDLKKVVKDH |
| | | AILDDIDLLNQIAEILTIYQDKDSIVAELGQLEYLMSEADKQSISELTGYTGTHSLSLK |
| | | CMNMIIDELWHSSMNQMEVFTYLNMRPKKYELKGYQRIPTDMIDDAILSPVVKRTF |
| | | IQSINVINKVIEKYGIPEDIIIELARENNSDDRKKFINNLQKKNEATRKRINEIIGQTGN |
| | | QNAKRIVEKIRLHDQQEGKCLYSLESIPLEDLLNNPNHYEVDHIIPRSVSFDNSYHNK |
| | | VLVKQSEASKKSNLTPYQYFNSGKSKLSYNQFKQHILNLSKSQDRISKKKKEYLLEE |
| | | RDINKFEVQKEFINRNLVDTRYATRELTSYLKAYFSANNMDVKVKTINGSFTNHLR |
| | | KVWRFDKYRNHGYKHHAEDALIIANADFLFKENKKLQNTNKILEKPTIENNTKKVT |
| | | VEKEEDYNNVFETPKLVEDIKQYRDYKFSHRVDKKPNRQLINDTLYSTRMKDEHDY |
| | | IVQTITDIYGKDNTNLKKQFNKNPEKFLMYQNDPKTFEKLSIIMKQYSDEKNPLAKY |
| | | YEETGEYLTKYSKKNNGPIVKKIKLLGNKVGNHLDVTNKYENSTKKLVKLSIKNYR |
| | | FDVYLTEKGYKFVTIAYLNVFKKDNYYYIPKDKYQELKEKKKIKDTDQFIASFYKN |
| | | DLIKLNGDLYKIIGVNSDDRNIIELDYYDIKYKDYCEINNIKGEPRIKKTIGKKTESIEK |
| | | FTTDVLGNLYLHSTEKAPQLIFKRGL |
| 3604 | Met(−) | NQKFILGLDIGITSVGYGLIDYETKNIIDAGVRLFPEANVENNEGRRSKRGSRRLKRR |
| | sRGN3.3(N584A)N | RIHRLERVKLLLTEYDLINKEQIPTSNNPYQIRVKGLSEILSKDELAIALLHLAKRRGI |
| | ickase | HNVDVAADKEETASDSLSTKDQINKNAKFLESRYVCELQKERLENEGHVRGVENRF |
| | | LTKDIVREAKKIIDTQMQYYPEIDETFKEKYISLVETRREYFEGPGQGSPFGWNGDLK |
| | | KWYEMLMGHCTYFPQELRSVKYAYSADLFNALNDLNNLIIQRDNSEKLEYHEKYHI |
| | | IENVFKQKKKPTLKQIAKEIGVNPEDIKGYRITKSGTPEFTSFKLFHDLKKVVKDHAI |
| | | LDDIDLLNQIAEILTIYQDKDSIVAELGQLEYLMSEADKQSISELTGYTGTHSLSLKC |
| | | MNMIIDELWHSSMNQMEVFTYLNMRPKKYELKGYQRIPTDMIDDAILSPVVKRTFI |
| | | QSINVINKVIEKYGIPEDIIIELARENNSDDRKKFINNLQKKNEATRKRINEIIGQTGNQ |
| | | NAKRIVEKIRLHDQQEGKCLYSLESIPLEDLLNNPNHYEVDHIIPRSVSFDNSYHNKV |
| | | LVKQSEASKKSNLTPYQYFNSGKSKLSYNQFKQHILNLSKSQDRISKKKKEYLLEER |
| | | DINKFEVQKEFINRNLVDTRYATRELTSYLKAYFSANNMDVKVKTINGSFTNHLRK |
| | | VWRFDKYRNHGYKHHAEDALIIANADFLFKENKKLQNTNKILEKPTIENNTKKVTV |
| | | EKEEDYNNVFETPKLVEDIKQYRDYKFSHRVDKKPNRQLINDTLYSTRMKDEHDYI |
| | | VQTITDIYGKDNTNLKKQFNKNPEKFLMYQNDPKTFEKLSIIMKQYSDEKNPLAKY |
| | | YEETGEYLTKYSKKNNGPIVKKIKLLGNKVGNHLDVTNKYENSTKKLVKLSIKNYR |
| | | FDVYLTEKGYKFVTIAYLNVFKKDNYYYIPKDKYQELKEKKKIKDTDQFIASFYKN |
| | | DLIKLNGDLYKIIGVNSDDRNIIELDYYDIKYKDYCEINNIKGEPRIKKTIGKKTESIEK |
| | | FTTDVLGNLYLHSTEKAPQLIFKRGL |
| 3605 | SpG | MDKKYSIGLDIGTNSVGWAVITDEYKVPSKKFKVLGNTDRHSIKKNLIGALLFDSGE |
| | | TAEATRLKRTARRRYTRRKNRICYLQEIFSNEMAKVDDSFFHRLEESFLVEEDKKHE |
| | | RHPIFGNIVDEVAYHEKYPTIYHLRKKLVDSTDKADLRLIYLALAHMIKFRGHFLIEG |
| | | DLNPDNSDVDKLFIQLVQTYNQLFEENPINASGVDAKAILSARLSKSRRLENLIAQLP |
| | | GEKKNGLFGNLIALSLGLTPNFKSNFDLAEDAKLQLSKDTYDDDLDNLLAQIGDQY |
| | | ADLFLAAKNLSDAILLSDILRVNTEITKAPLSASMIKRYDEHHQDLTLLKALVRQQLP |
| | | EKYKEIFFDQSKNGYAGYIDGGASQEEFYKFIKPILEKMDGTEELLVKLNREDLLRK |
| | | QRTFDNGSIPHQIHLGELHAILRRQEDFYPFLKDNREKIEKILTFRIPYYVGPLARGNS |
| | | RFAWMTRKSEETITPWNFEEVVDKGASAQSFIERMTNFDKNLPNEKVLPKHSLLYE |
| | | YFTVYNELTKVKYVTEGMRKPAFLSGEQKKAIVDLLFKTNRKVTVKQLKEDYFKKI |
| | | ECFDSVEISGVEDRFNASLGTYHDLLKIIKDKDFLDNEENEDILEDIVLTLTLFEDREM |
| | | IEERLKTYAHLFDDKVMKQLKRRRYTGWGRLSRKLINGIRDKQSGKTILDFLKSDGF |
| | | ANRNFMQLIHDDSLTFKEDIQKAQVSGQGDSLHEHIANLAGSPAIKKGILQTVKVVD |
| | | ELVKVMGRHKPENIVIEMARENQTTQKGQKNSRERMKRIEEGIKELGSQILKEHPVE |
| | | NTQLQNEKLYLYYLQNGRDMYVDQELDINRLSDYDVDHIVPQSFLKDDSIDNKVLT |
| | | RSDKNRGKSDNVPSEEVVKKMKNYWRQLLNAKLITQRKFDNLTKAERGGLSELDK |
| | | AGFIKRQLVETRQITKHVAQILDSRMNTKYDENDKLIREVKVITLKSKLVSDFRKDF |
| | | QFYKVREINNYHHAHDAYLNAVVGTALIKKYPKLESEFVYGDYKVYDVRKMIAKS |
| | | EQEIGKATAKYFFYSNIMNFFKTEITLANGEIRKRPLIETNGETGEIVWDKGRDFATV |
| | | RKVLSMPQVNIVKKTEVQTGGFSKESILPKRNSDKLIARKKDWDPKKYGGFLWPTV |
| | | AYSVLVVAKVEKGKSKKLKSVKELLGITIMERSSFEKNPIDFLEAKGYKEVKKDLIIK |
| | | LPKYSLFELENGRKRMLASAKQLQKGNELALPSKYVNFLYLASHYEKLKGSPEDNE |
| 3606 | SpG(H840A) | QKQLFVEQHKHYLDEIIEQISEFSKRVILADANLDKVLSAYNKHRDKPIREQAENIIH |
| | Nickase | LFTLTNLGAPAAFKYFDTTIDRKQYRSTKEVLDATLIHQSITGLYETRIDLSQLGGD |
| | | MDKKYSIGLDIGTNSVGWAVITDEYKVPSKKFKVLGNTDRHSIKKNLIGALLFDSGE |
| | | TAEATRLKRTARRRYTRRKNRICYLQEIFSNEMAKVDDSFFHRLEESFLVEEDKKHE |
| | | RHPIFGNIVDEVAYHEKYPTIYHLRKKLVDSTDKADLRLIYLALAHMIKFRGHFLIEG |
| | | DLNPDNSDVDKLFIQLVQTYNQLFEENPINASGVDAKAILSARLSKSRRLENLIAQLP |
| | | GEKKNGLFGNLIALSLGLTPNFKSNFDLAEDAKLQLSKDTYDDDLDNLLAQIGDQY |
| | | ADLFLAAKNLSDAILLSDILRVNTEITKAPLSASMIKRYDEHHQDLTLLKALVRQQLP |
| | | EKYKEIFFDQSKNGYAGYIDGGASQEEFYKFIKPILEKMDGTEELLVKLNREDLLRK |
| | | QRTFDNGSIPHQIHLGELHAILRRQEDFYPFLKDNREKIEKILTFRIPYYVGPLARGNS |
| | | RFAWMTRKSEETITPWNFEEVVDKGASAQSFIERMTNFDKNLPNEKVLPKHSLLYE |
| | | YFTVYNELTKVKYVTEGMRKPAFLSGEQKKAIVDLLFKTNRKVTVKQLKEDYFKKI |
| | | ECFDSVEISGVEDRFNASLGTYHDLLKIIKDKDFLDNEENEDILEDIVLTLTLFEDREM |
| | | IEERLKTYAHLFDDKVMKQLKRRRYTGWGRLSRKLINGIRDKQSGKTILDFLKSDGF |
| | | ANRNFMQLIHDDSLTFKEDIQKAQVSGQGDSLHEHIANLAGSPAIKKGILQTVKVVD |
| | | ELVKVMGRHKPENIVIEMARENQTTQKGQKNSRERMKRIEEGIKELGSQILKEHPVE |
| | | NTQLQNEKLYLYYLQNGRDMYVDQELDINRLSDYDVDAIVPQSFLKDDSIDNKVLT |
| | | RSDKNRGKSDNVPSEEVVKKMKNYWRQLLNAKLITQRKFDNLTKAERGGLSELDK |
| | | AGFIKRQLVETRQITKHVAQILDSRMNTKYDENDKLIREVKVITLKSKLVSDFRKDF |
| | | QFYKVREINNYHHAHDAYLNAVVGTALIKKYPKLESEFVYGDYKVYDVRKMIAKS |
| | | EQEIGKATAKYFFYSNIMNFFKTEITLANGEIRKRPLIETNGETGEIVWDKGRDFATV |
| 3607 | Met(−) | RKVLSMPQVNIVKKTEVQTGGFSKESILPKRNSDKLIARKKDWDPKKYGGFLWPTV |
| | SpG(H839A) | AYSVLVVAKVEKGKSKKLKSVKELLGITIMERSSFEKNPIDFLEAKGYKEVKKDLIIK |
| | Nickase | LPKYSLFELENGRKRMLASAKQLQKGNELALPSKYVNFLYLASHYEKLKGSPEDNE |
| | | QKQLFVEQHKHYLDEIIEQISEFSKRVILADANLDKVLSAYNKHRDKPIREQAENIIH |
| | | LFTLTNLGAPAAFKYFDTTIDRKQYRSTKEVLDATLIHQSITGLYETRIDLSQLGGD |
| | | DKKYSIGLDIGTNSVGWAVITDEYKVPSKKFKVLGNTDRHSIKKNLIGALLFDSGET |
| | | AEATRLKRTARRRYTRRKNRICYLQEIFSNEMAKVDDSFFHRLEESFLVEEDKKHER |
| | | HPIFGNIVDEVAYHEKYPTIYHLRKKLVDSTDKADLRLIYLALAHMIKFRGHFLIEGD |
| | | LNPDNSDVDKLFIQLVQTYNQLFEENPINASGVDAKAILSARLSKSRRLENLIAQLPG |
| | | EKKNGLFGNLIALSLGLTPNFKSNFDLAEDAKLQLSKDTYDDDLDNLLAQIGDQYA |
| | | DLFLAAKNLSDAILLSDILRVNTEITKAPLSASMIKRYDEHHQDLTLLKALVRQQLPE |
| | | KYKEIFFDQSKNGYAGYIDGGASQEEFYKFIKPILEKMDGTEELLVKLNREDLLRKQ |
| | | RTFDNGSIPHQIHLGELHAILRRQEDFYPFLKDNREKIEKILTFRIPYYVGPLARGNSR |
| | | FAWMTRKSEETITPWNFEEVVDKGASAQSFIERMTNFDKNLPNEKVLPKHSLLYEY |
| | | FTVYNELTKVKYVTEGMRKPAFLSGEQKKAIVDLLFKTNRKVTVKQLKEDYFKKIE |
| | | CFDSVEISGVEDRFNASLGTYHDLLKIIKDKDFLDNEENEDILEDIVLTLTLFEDREMI |
| | | EERLKTYAHLFDDKVMKQLKRRRYTGWGRLSRKLINGIRDKQSGKTILDFLKSDGF |
| | | ANRNFMQLIHDDSLTFKEDIQKAQVSGQGDSLHEHIANLAGSPAIKKGILQTVKVVD |
| | | ELVKVMGRHKPENIVIEMARENQTTQKGQKNSRERMKRIEEGIKELGSQILKEHPVE |
| | | NTQLQNEKLYLYYLQNGRDMYVDQELDINRLSDYDVDAIVPQSFLKDDSIDNKVLT |
| | | RSDKNRGKSDNVPSEEVVKKMKNYWRQLLNAKLITQRKFDNLTKAERGGLSELDK |
| | | AGFIKRQLVETRQITKHVAQILDSRMNTKYDENDKLIREVKVITLKSKLVSDFRKDF |
| | | QFYKVREINNYHHAHDAYLNAVVGTALIKKYPKLESEFVYGDYKVYDVRKMIAKS |
| | | EQEIGKATAKYFFYSNIMNFFKTEITLANGEIRKRPLIETNGETGEIVWDKGRDFATV |
| | | RKVLSMPQVNIVKKTEVQTGGFSKESILPKRNSDKLIARKKDWDPKKYGGFLWPTV |
| | | AYSVLVVAKVEKGKSKKLKSVKELLGITIMERSSFEKNPIDFLEAKGYKEVKKDLIIK |
| | | LPKYSLFELENGRKRMLASAKQLQKGNELALPSKYVNFLYLASHYEKLKGSPEDNE |
| | | QKQLFVEQHKHYLDEIIEQISEFSKRVILADANLDKVLSAYNKHRDKPIREQAENIIH |
| | | LFTLTNLGAPAAFKYFDTTIDRKQYRSTKEVLDATLIHQSITGLYETRIDLSQLGGD |
| | saCas9 PAM | NNGRRT |
| | saCas9 PAM | NNGRRN |
| | saCas9-KKH | NNNRRT |
| | (E782K, N968K, | |
| | R1015H) PAM | |
| | NmCas9 PAM | NNNNGATT |
| | StCas9 PAM | NNAGAAW |
| | TdCas9 PAM | NAAAAC |
| 3608 | NLS of SV40 Large | PKKKRKV |
| | T-AG | |
| 3609 | N-terminal NLS | MKRTADGSEFESPKKKRKV |
| | and Linker for | |
| | Exemplary Prime | |
| | Editor | |
| 3610 | NLS | MDSLLMNRRKFLYQFKNVRWAKGRRETYLC |
| 3611 | NLS of | AVKRPAATKKAGQAKKKKLD |
| | Nucleoplasmin | |
| 3612 | NLS of EGL-13 | MSRRRKANPTKLSENAKKLAKEVEN |
| 3613 | NLS of C-Myc | PAAKRVKLD |
| 3614 | NLS of Tus-protein | KLKIKRPVK |
| 3615 | NLS of polyoma | VSRKRPRP |
| | large T-AG | |
| 3616 | NLS of Hepatitis D | EGAPPAKRAR |
| | virus antigen | |
| 3617 | NLS of murine p53 | PPQPKKKPLDGE |
| 3618 | C-Terminal linker | SGGSKRTADGSEFEPKKKRKV |
| | and NLS for | |
| | Exemplary Prime | |
| | Editor | |
| 3619 | MS2 aptamer | GCCAACATGAGGATCACCCATGTCTGCAGGGCC |
| 3620 | MS2cp | GSASNFTQFVLVDNGGTGDVTVAPSNFANGVAEWISSNSRSQAYKVTCSVRQSSAQ |
| | | NRKYTIKVEVPKVATQTVGGEELPVAGWRSYLNMELTIPIFATNSDCELIVKAMQGL |
| | | LKDGNPIPSAIAANSGIY |
| 3621 | Exemplary Prime | MKRTADGSEFESPKKKRKVDKKYSIGLDIGTNSVGWAVITDEYKVPSKKFKVLGNT |
| | Editor [NLS]- | DRHSIKKNLIGALLFDSGETAEATRLKRTARRRYTRRKNRICYLQEIFSNEMAKVDD |
| | [Cas9(H840A)]- | SFFHRLEESFLVEEDKKHERHPIFGNIVDEVAYHEKYPTIYHLRKKLVDSTDKADLRL |
| | [linker]- | IYLALAHMIKFRGHFLIEGDLNPDNSDVDKLFIQLVQTYNQLFEENPINASGVDAKAI |
| | [MMLV_RT(D200N) | LSARLSKSRRLENLIAQLPGEKKNGLFGNLIALSLGLTPNFKSNFDLAEDAKLQLSKD |
| | (T330P)(L603W) | TYDDDLDNLLAQIGDQYADLFLAAKNLSDAILLSDILRVNTEITKAPLSASMIKRYDE |
| | (T306K)(W313F)]- | HHQDLTLLKALVRQQLPEKYKEIFFDQSKNGYAGYIDGGASQEEFYKFIKPILEKMD |
| | [NLS] | GTEELLVKLNREDLLRKQRTFDNGSIPHQIHLGELHAILRRQEDFYPFLKDNREKIEKI |
| | | LTFRIPYYVGPLARGNSRFAWMTRKSEETITPWNFEEVVDKGASAQSFIERMTNFDK |
| | | NLPNEKVLPKHSLLYEYFTVYNELTKVKYVTEGMRKPAFLSGEQKKAIVDLLFKTN |
| | | RKVTVKQLKEDYFKKIECFDSVEISGVEDRFNASLGTYHDLLKIIKDKDFLDNEENE |
| | | DILEDIVLTLTLFEDREMIEERLKTYAHLFDDKVMKQLKRRRYTGWGRLSRKLINGI |
| | | RDKQSGKTILDFLKSDGFANRNFMQLIHDDSLTFKEDIQKAQVSGQGDSLHEHIANL |
| | | AGSPAIKKGILQTVKVVDELVKVMGRHKPENIVIEMARENQTTQKGQKNSRERMKR |
| | | IEEGIKELGSQILKEHPVENTQLQNEKLYLYYLQNGRDMYVDQELDINRLSDYDVD |
| | | AIVPQSFLKDDSIDNKVLTRSDKNRGKSDNVPSEEVVKKMKNYWRQLLNAKLITQR |
| | | KFDNLTKAERGGLSELDKAGFIKRQLVETRQITKHVAQILDSRMNTKYDENDKLIRE |
| | | VKVITLKSKLVSDFRKDFQFYKVREINNYHHAHDAYLNAVVGTALIKKYPKLESEF |
| | | VYGDYKVYDVRKMIAKSEQEIGKATAKYFFYSNIMNFFKTEITLANGEIRKRPLIETN |
| | | GETGEIVWDKGRDFATVRKVLSMPQVNIVKKTEVQTGGFSKESILPKRNSDKLIARK |
| | | KDWDPKKYGGFDSPTVAYSVLVVAKVEKGKSKKLKSVKELLGITIMERSSFEKNPI |
| | | DFLEAKGYKEVKKDLIIKLPKYSLFELENGRKRMLASAGELQKGNELALPSKYVNFL |
| | | YLASHYEKLKGSPEDNEQKQLFVEQHKHYLDEIIEQISEFSKRVILADANLDKVLSA |
| | | YNKHRDKPIREQAENIIHLFTLTNLGAPAAFKYFDTTIDRKRYTSTKEVLDATLIHQSI |
| | | TGLYETRIDLSQLGGDSGGSSGGSSGSETPGTSESATPESSGGSSGGSSTLNIEDEYRL |
| | | HETSKEPDVSLGSTWLSDFPQAWAETGGMGLAVRQAPLIIPLKATSTPVSIKQYPMS |
| | | QEARLGIKPHIQRLLDQGILVPCQSPWNTPLLPVKKPGTNDYRPVQDLREVNKRVED |
| | | IHPTVPNPYNLLSGLPPSHQWYTVLDLKDAFFCLRLHPTSQPLFAFEWRDPEMGISG |
| | | QLTWTRLPQGFKNSPTLFNEALHRDLADFRIQHPDLILLQYVDDLLLAATSELDCQQ |
| | | GTRALLQTLGNLGYRASAKKAQICQKQVKYLGYLLKEGQRWLTEARKETVMGQPT |
| | | PKTPRQLREFLGKAGFCRLFIPGFAEMAAPLYPLTKPGTLFNWGPDQQKAYQEIKQA |
| | | LLTAPALGLPDLTKPFELFVDEKQGYAKGVLTQKLGPWRRPVAYLSKKLDPVAAG |
| | | WPPCLRMVAAIAVLTKDAGKLTMGQPLVILAPHAVEALVKQPPDRWLSNARMTHY |
| | | QALLLDTDRVQFGPVVALNPATLLPLPEEGLQHNCLDILAEAHGTRPDLTDQPLPDA |
| | | DHTWYTDGSSLLQEGQRKAGAAVTTETEVIWAKALPAGTSAQRAELIALTQALKM |
| | | AEGKKLNVYTDSRYAFATAHIHGEIYRRRGWLTSEGKEIKNKDEILALLKALFLPKR |
| | | LSIIHCPGHQKGHSAEARGNRMADQAARKAAITETPDTSTLLIENSSPSGGSKRTADG |
| | | SEFEPKKKRKV |
| 3622 | Pemax Prime | MKRTADGSEFESPKKKRKVDKKYSIGLDIGTNSVGWAVITDEYKVPSKKFKVLGNT |
| | Editor | DRHSIKKNLIGALLFDSGETAEATRLKRTARRRYTRRKNRICYLQEIFSNEMAKVDD |
| | | SFFHRLEESFLVEEDKKHERHPIFGNIVDEVAYHEKYPTIYHLRKKLVDSTDKADLRL |
| | | IYLALAHMIKFRGHFLIEGDLNPDNSDVDKLFIQLVQTYNQLFEENPINASGVDAKAI |
| | | LSARLSKSRKLENLIAQLPGEKKNGLFGNLIALSLGLTPNFKSNFDLAEDAKLQLSKD |
| | | TYDDDLDNLLAQIGDQYADLFLAAKNLSDAILLSDILRVNTEITKAPLSASMIKRYDE |
| | | HHQDLTLLKALVRQQLPEKYKEIFFDQSKNGYAGYIDGGASQEEFYKFIKPILEKMD |
| | | GTEELLVKLKREDLLRKQRTFDNGSIPHQIHLGELHAILRRQEDFYPFLKDNREKIEKI |
| | | LTFRIPYYVGPLARGNSRFAWMTRKSEETITPWNFEEVVDKGASAQSFIERMTNFDK |
| | | NLPNEKVLPKHSLLYEYFTVYNELTKVKYVTEGMRKPAFLSGEQKKAIVDLLFKTN |
| | | RKVTVKQLKEDYFKKIECFDSVEISGVEDRFNASLGTYHDLLKIIKDKDFLDNEENE |
| | | DILEDIVLTLTLFEDREMIEERLKTYAHLFDDKVMKQLKRRRYTGWGRLSRKLINGI |
| | | RDKQSGKTILDFLKSDGFANRNFMQLIHDDSLTFKEDIQKAQVSGQGDSLHEHIANL |
| | | AGSPAIKKGILQTVKVVDELVKVMGRHKPENIVIEMARENQTTQKGQKNSRERMKR |
| | | IEEGIKELGSQILKEHPVENTQLQNEKLYLYYLQNGRDMYVDQELDINRLSDYDVD |
| | | AIVPQSFLKDDSIDNKVLTRSDKNRGKSDNVPSEEVVKKMKNYWRQLLNAKLITQR |
| | | KFDNLTKAERGGLSELDKAGFIKRQLVETRQITKHVAQILDSRMNTKYDENDKLIRE |
| | | VKVITLKSKLVSDFRKDFQFYKVREINNYHHAHDAYLNAVVGTALIKKYPKLESEF |
| | | VYGDYKVYDVRKMIAKSEQEIGKATAKYFFYSNIMNFFKTEITLANGEIRKRPLIETN |
| | | GETGEIVWDKGRDFATVRKVLSMPQVNIVKKTEVQTGGFSKESILPKRNSDKLIARK |
| | | KDWDPKKYGGFDSPTVAYSVLVVAKVEKGKSKKLKSVKELLGITIMERSSFEKNPI |
| | | DFLEAKGYKEVKKDLIIKLPKYSLFELENGRKRMLASAGELQKGNELALPSKYVNFL |
| | | YLASHYEKLKGSPEDNEQKQLFVEQHKHYLDEIIEQISEFSKRVILADANLDKVLSA |
| | | YNKHRDKPIREQAENIIHLFTLTNLGAPAAFKYFDTTIDRKRYTSTKEVLDATLIHQSI |
| | | TGLYETRIDLSQLGGDSGGSSGGSKRTADGSEFESPKKKRKVSGGSSGGSTLNIEDEY |
| | | RLHETSKEPDVSLGSTWLSDFPQAWAETGGMGLAVRQAPLIIPLKATSTPVSIKQYP |
| | | MSQEARLGIKPHIQRLLDQGILVPCQSPWNTPLLPVKKPGTNDYRPVQDLREVNKRV |
| | | EDIHPTVPNPYNLLSGLPPSHQWYTVLDLKDAFFCLRLHPTSQPLFAFEWRDPEMGIS |
| | | GQLTWTRLPQGFKNSPTLFNEALHRDLADFRIQHPDLILLQYVDDLLLAATSELDCQ |
| | | QGTRALLQTLGNLGYRASAKKAQICQKQVKYLGYLLKEGQRWLTEARKETVMGQ |
| | | PTPKTPRQLREFLGKAGFCRLFIPGFAEMAAPLYPLTKPGTLFNWGPDQQKAYQEIK |
| | | QALLTAPALGLPDLTKPFELFVDEKQGYAKGVLTQKLGPWRRPVAYLSKKLDPVA |
| | | AGWPPCLRMVAAIAVLTKDAGKLTMGQPLVILAPHAVEALVKQPPDRWLSNARMT |
| | | HYQALLLDTDRVQFGPVVALNPATLLPLPEEGLQHNCLDILAEAHGTRPDLTDQPLP |
| | | DADHTWYTDGSSLLQEGQRKAGAAVTTETEVIWAKALPAGTSAQRAELIALTQAL |
| | | KMAEGKKLNVYTDSRYAFATAHIHGEIYRRRGWLTSEGKEIKNKDEILALLKALFLP |
| | | KRLSIIHCPGHQKGHSAEARGNRMADQAARKAAITETPDTSTLLIENSSPSGGSKRTA |
| | | DGSEFESPKKKRKVGSGPAAKRVKLD |
| | PEmax-N- | MKRTADGSEFESPKKKRKV |
| | terminal | |
| | bpSV40NLS | |
| 3623 | PEmax-CAS9 | DKKYSIGLDIGTNSVGWAVITDEYKVPSKKFKVLGNTDRHSIKKNLIGALLFDSGET |
| | (R221K N394K | AEATRLKRTARRRYTRRKNRICYLQEIFSNEMAKVDDSFFHRLEESFLVEEDKKHER |
| | H840A) (not | HPIFGNIVDEVAYHEKYPTIYHLRKKLVDSTDKADLRLIYLALAHMIKFRGHFLIEGD |
| | including N- | LNPDNSDVDKLFIQLVQTYNQLFEENPINASGVDAKAILSARLSKSRKLENLIAQLPG |
| | terminal Met in | EKKNGLFGNLIALSLGLTPNFKSNFDLAEDAKLQLSKDTYDDDLDNLLAQIGDQYA |
| | Cas9) | DLFLAAKNLSDAILLSDILRVNTEITKAPLSASMIKRYDEHHQDLTLLKALVRQQLPE |
| | | KYKEIFFDQSKNGYAGYIDGGASQEEFYKFIKPILEKMDGTEELLVKLKREDLLRKQ |
| | | RTFDNGSIPHQIHLGELHAILRRQEDFYPFLKDNREKIEKILTFRIPYYVGPLARGNSR |
| | | FAWMTRKSEETITPWNFEEVVDKGASAQSFIERMTNFDKNLPNEKVLPKHSLLYEY |
| | | FTVYNELTKVKYVTEGMRKPAFLSGEQKKAIVDLLFKTNRKVTVKQLKEDYFKKIE |
| | | CFDSVEISGVEDRFNASLGTYHDLLKIIKDKDFLDNEENEDILEDIVLTLTLFEDREMI |
| | | EERLKTYAHLFDDKVMKQLKRRRYTGWGRLSRKLINGIRDKQSGKTILDFLKSDGF |
| | | ANRNFMQLIHDDSLTFKEDIQKAQVSGQGDSLHEHIANLAGSPAIKKGILQTVKVVD |
| | | ELVKVMGRHKPENIVIEMARENQTTQKGQKNSRERMKRIEEGIKELGSQILKEHPVE |
| | | NTQLQNEKLYLYYLQNGRDMYVDQELDINRLSDYDVDAIVPQSFLKDDSIDNKVLT |
| | | RSDKNRGKSDNVPSEEVVKKMKNYWRQLLNAKLITQRKFDNLTKAERGGLSELDK |
| | | AGFIKRQLVETRQITKHVAQILDSRMNTKYDENDKLIREVKVITLKSKLVSDFRKDF |
| | | QFYKVREINNYHHAHDAYLNAVVGTALIKKYPKLESEFVYGDYKVYDVRKMIAKS |
| | | EQEIGKATAKYFFYSNIMNFFKTEITLANGEIRKRPLIETNGETGEIVWDKGRDFATV |
| | | RKVLSMPQVNIVKKTEVQTGGFSKESILPKRNSDKLIARKKDWDPKKYGGFDSPTV |
| | | AYSVLVVAKVEKGKSKKLKSVKELLGITIMERSSFEKNPIDFLEAKGYKEVKKDLIIK |
| | | LPKYSLFELENGRKRMLASAGELQKGNELALPSKYVNFLYLASHYEKLKGSPEDNE |
| | | QKQLFVEQHKHYLDEIIEQISEFSKRVILADANLDKVLSAYNKHRDKPIREQAENIIH |
| | | LFTLTNLGAPAAFKYFDTTIDRKRYTSTKEVLDATLIHQSITGLYETRIDLSQLGGD |
| 3624 | PEmax-SGGSx2- | SGGSSGGSKRTADGSEFESPKKKRKVSGGSSGGS |
| | bpSV40NLS- | |
| | SGGSx2 linker | |
| | PEmax- | TLNIEDEYRLHETSKEPDVSLGSTWLSDFPQAWAETGGMGLAVRQAPLIIPLKATST |
| | MMLV RT D200N | PVSIKQYPMSQEARLGIKPHIQRLLDQGILVPCQSPWNTPLLPVKKPGTNDYRPVQD |
| | T330P L603W | LREVNKRVEDIHPTVPNPYNLLSGLPPSHQWYTVLDLKDAFFCLRLHPTSQPLFAFE |
| | T306K W313F | WRDPEMGISGQLTWTRLPQGFKNSPTLFNEALHRDLADFRIQHPDLILLQYVDDLLL |
| | | AATSELDCQQGTRALLQTLGNLGYRASAKKAQICQKQVKYLGYLLKEGQRWLTEA |
| | | RKETVMGQPTPKTPRQLREFLGKAGFCRLFIPGFAEMAAPLYPLTKPGTLFNWGPDQ |
| | | QKAYQEIKQALLTAPALGLPDLTKPFELFVDEKQGYAKGVLTQKLGPWRRPVAYLS |
| | | KKLDPVAAGWPPCLRMVAAIAVLTKDAGKLTMGQPLVILAPHAVEALVKQPPDRW |
| | | LSNARMTHYQALLLDTDRVQFGPVVALNPATLLPLPEEGLQHNCLDILAEAHGTRP |
| | | DLTDQPLPDADHTWYTDGSSLLQEGQRKAGAAVTTETEVIWAKALPAGTSAQRAE |
| | | LIALTQALKMAEGKKLNVYTDSRYAFATAHIHGEIYRRRGWLTSEGKEIKNKDEILA |
| | | LLKALFLPKRLSIIHCPGHQKGHSAEARGNRMADQAARKAAITETPDTSTLLIENSSP |
| 3625 | PEmax-C-terminal | SGGSKRTADGSEFESPKKKRKV |
| | linker-NLS | |
| 3626 | PEmax-C-terminal | GSGPAAKRVKLD |
| | linker-NLS2 | |
| 3627 | Linker | (GGGGS)n |
| 3628 | Linker | (G)n |
| 3629 | Linker | (EAAAK)n |
| 3630 | Linker | (GGS)n |
| 3631 | Linker | (SGGS)n |
| 3632 | Linker | (XP)n |
| 3633 | Linker | SGSETPGTSESATPES |
| 3634 | Linker | SGGSSGGSSGSETPGTSESATPESSGGSSGGS |
| 3635 | Linker between | SGGSSGGSSGSETPGTSESATPESSGGSSGGSS |
| | Cas9 domain and | |
| | RT domain in | |
| | Exemplary Prime | |
| | Editor | |
| 3636 | Linker | SGGSGGSGGS |
| 3637 | Linker | SGGS |
| 3638 | Linker | SGGSSGGSSGSETPGTSESATPESAGSYPYDVPDYAGSAAPAAKKKKLDGSGSGGSS |
| | | GGS |
| 3639 | Linker | GGSGGS |
| 3640 | Linker | GGSGGSGGS |
| 3641 | gRNA core | GTTTTAGAGCTAGAAATAGCAAGTTAAAATAAGGCTAGTCCGTTATCAACTTGA |
| | | AAAAGTGGCACCGAGTCGGTGC |
| 3642 | gRNA core | GTTTGAGAGCTAGAAATAGCAAGTTTAAATAAGGCTAGTCCGTTATCAACTTGA |
| | | AAAAGTGGGACCGAGTCGGTCC |
| 3643 | gRNA core | GTTTAAGAGCTATGCTGGAAACAGCATAGCAAGTTTAAATAAGGCTAGTCCGTT |
| | | ATCAACTTGAAAAAGTGGCACCGAGTCGGTGC |
| 3644 | SaCas9 gRNA core | GTTTTAGTACTCTGGAAACAGAATCTACTAAAACAAGGCAAAATGCCGTGTTTA |
| | | TCTCGTCAACTTGTTGGCGAGA |
| 3645 | SluCas9 gRNA | GTTTTAGTACTCTGGAAACAGAATCTACTGAAACAAGACAATATGTCGTGTTTAT |
| | core | CCCATCAATTTATTGGTGGGA |

LENGTHY TABLES
The patent application contains a lengthy table section. A copy of the table is available in electronic form from the USPTO web site (https://seqdata.uspto.gov/?pageRequest=docDetail&DocID=US20240360476A1). An electronic copy of the table will also be available from the USPTO upon request and payment of the fee set forth in 37 CFR 1.19(b)(3).

What is claimed is:

1. A prime editing composition comprising (A) a first prime editing guide RNA (PEgRNA) or one or more polynucleotides encoding the first PEgRNA and (B) a second PEgRNA or one or more polynucleotides encoding the second PEgRNA,
wherein the first PEgRNA comprises:
(i) a first spacer that is complementary to a first search target sequence on a first strand of a DMPK gene,
(ii) a first gRNA core capable of binding to a Cas9 protein; and
(iii) a first extension arm comprising a first editing template and a first primer binding site (PBS),
wherein the first spacer comprises at its 3′ end nucleotides 5-20 of a sequence selected from the group consisting of SEQ ID NOs: 1, 20, 39, 58, 77, 484, 518, 536, 554, 572, 590, 912, 929, 947, 965, 983, 1001, 1019, and 1341, and wherein the first PBS comprises at its 5′ end a sequence that is the reverse complement of nucleotides 15-17 of the selected sequence;
wherein the second PEgRNA comprises:
(i) a second spacer that is complementary to a second search target sequence on a second strand of the DMPK gene complementary to the first strand,
(ii) a second gRNA core capable of binding to a Cas9 protein; and
(iii) a second extension arm comprising a second editing template and a second PBS,
wherein the second spacer comprises at its 3′ end nucleotides 5-20 of a sequence selected from the group consisting of SEQ ID NOs: 1359, 1381, 1403, 1503, 1525, 1864, 1882, 1900, 1918, 1936, 2263, 2281, 2299, 2317, 2335, 2353, 2673, and wherein the second PBS comprises at its 5′ end a sequence that is the reverse complement of nucleotides 15-17 of the selected sequence;
and wherein
(a) the first editing template comprises a region of complementarity to the second editing template;
(b) the first editing template comprises nucleotides 8-17 of the selected sequence for the second spacer, and the second editing template comprises nucleotides 8-17 of the selected sequence for the first spacer; or
(c) the first editing template comprises nucleotides 8-17 of the selected sequence for the second spacer, and a region of complementarity to the second editing template, and the second editing template comprises nucleotides 8-17 of the selected sequence for the first spacer, and a region of complementarity to the first editing template.

2. The prime editing composition of claim 1, wherein the selected sequence for the first spacer is SEQ ID NOs: 77, 484, 536, 590, or 1019.

3. The prime editing composition of claim 1 or 2, wherein the selected sequence for the second spacer is SEQ ID NO: 1525, 1900, 1936, 2263, 2281, 2299, 2353, or 2673.

4. The prime editing composition of any one of claims 1-3, wherein the selected sequence for the first spacer is SEQ ID NO: 77, 536, or 1019.

5. The prime editing composition of any one of claims 1-4, wherein the selected sequence for the second spacer is SEQ ID NO: 1900, 1936, or 2673.

6. The prime editing composition of any one of claims 1-5, wherein the first spacer and/or the second spacer is from 16 to 22 nucleotides in length.

7. The prime editing composition of any one of claims 1-6, wherein the first spacer and/or the second spacer comprises at its 3′ the selected sequence.

8. The prime editing composition of claim 7, wherein the first spacer and/or the second spacer is 20 nucleotides in length and comprises the selected sequence.

9. The prime editing composition of any one of claims 1-8, wherein the first gRNA core and the second gRNA core comprise the same sequence.

10. The prime editing composition of claim 9, wherein the first gRNA core and the second gRNA core each comprises SEQ ID NO: 3641.

11. The prime editing composition of any one of claims 1-10, wherein the first spacer, the first gRNA core, the first editing template, and the first PBS form a contiguous sequence in a single molecule.

12. The prime editing composition of claim 11, wherein the first PEgRNA comprises from 5′ to 3′ the first spacer, the first gRNA core, the first editing template, and the first PBS.

13. The prime editing composition of any one of claims 1-12, wherein the second spacer, the second gRNA core, the second editing template, and the second PBS form a contiguous sequence in a single molecule.

14. The prime editing composition of claim 13, wherein the second pegRNA comprises from 5′ to 3′ the second spacer, the second gRNA core, the second editing template, and the second PBS.

15. The prime editing composition of any one of claims 1-14, where in the first PBS is at least 8 nucleotides in length and comprises at its 5′ end a sequence that is the reverse complement of nucleotides 10-17 of the selected sequence for the first spacer.

16. The prime editing composition of claim 15, wherein the first PBS is 8-17 nucleotides in length and comprises at its 5′ end a sequence that is the reverse complement of nucleotides 10-17, 9-17, 8-17, 7-17, 6-17, 5-17, 4-17, 3-17, 2-17, or 1-17 of the selected sequence for the first spacer.

17. The prime editing composition of claim 16, wherein the first PBS is 8-14 nucleotides in length.

18. The prime editing composition of claim 16, wherein the first PBS is 8-12 nucleotides in length.

19. The prime editing composition of claim 16, wherein the first PBS is 8-10 nucleotides in length.

20. The prime editing composition of claim 16, wherein the first PBS is 8-9 nucleotides in length.

21. The prime editing composition of claim 16, wherein the first PBS is 10 nucleotides in length.

22. The prime editing composition of any one of claims 1-21, where in the second PBS is at least 8 nucleotides in length and comprises at its 5′ end a sequence that is the reverse complement of nucleotides 10-17 of the selected sequence for the second spacer.

23. The prime editing composition of claim 22, wherein the second PBS is 8-17 nucleotides in length and comprises at its 5′ end a sequence that is the reverse complement of nucleotides 10-17, 9-17, 8-17, 7-17, 6-17, 5-17, 4-17, 3-17, 2-17, or 1-17 of the selected sequence for the second spacer.

24. The prime editing composition of claim 23, wherein the second PBS is 8-14 nucleotides in length.

25. The prime editing composition of claim 23, wherein the second PBS is 8-12 nucleotides in length.

26. The prime editing composition of claim 23, wherein the second PBS is 8-10 nucleotides in length.

27. The prime editing composition of claim 23, wherein the second PBS is 8-9 nucleotides in length.

28. The prime editing composition of claim 23, wherein the second PBS is 10 nucleotides in length.

29. The prime editing composition of any one of claims 1-28, wherein the first editing template comprises a region of complementarity to the second editing template.

30. The prime editing composition of claim 29, wherein the region of complementarity is about 20 to about 80 nucleotides in length.

31. The prime editing composition of claim 29, wherein the region of complementarity is about 20 to about 40 nucleotides in length.

32. The prime editing composition of claim 29, wherein the region of complementarity is 23-38 nucleotides in length.

33. The prime editing composition of claim 29, wherein the GC content of the region of complementarity is about 40% to about 80%.

34. The prime editing composition of claim 29, wherein the GC content of the region of complementarity is about 50% to about 80%.

35. The prime editing composition of claim 29, wherein the GC content of the region of complementarity is about 60% to about 80%.

36. The prime editing composition of claim 29, wherein the GC content of the region of complementarity is at least 63%.

37. The prime editing composition of claim 29, wherein the GC content of the region of complementarity is 63%-79%.

38. The prime editing composition of any one of claims 29-37, wherein the first editing template comprises SEQ ID NO: 2691.

39. The prime editing composition of any one of claims 29-38, wherein the second editing template comprises SEQ ID NO: 3075.

40. The prime editing composition of any one of claims 29-37, where in the first editing template comprises a sequence selected from the group consisting of SEQ ID NOs: 2692-2736.

41. The prime editing composition of any one of claims 29-37 and 40, wherein the second editing template comprises a sequence selected from the group consisting of SEQ ID NOs: 3076-3120.

42. The prime editing composition of any one of claims 29-37, wherein the first editing template comprises a sequence selected from the group consisting of SEQ ID NOs: 2737-2769.

43. The prime editing composition of any one of claims 29-37 and 42, wherein the second editing template comprises a sequence selected from the group consisting of SEQ ID NOs: 3121-3153.

44. The prime editing composition of any one of claims 29-43, wherein:
(i) the first spacer comprises SEQ ID NO: 77, and the first PBS comprises SEQ ID No: 85,
(ii) the first spacer comprises SEQ ID NO: 536, and the first PBS comprises SEQ ID No: 544,
or
(iii) the first spacer comprises SEQ ID NO: 1019, and the first PBS comprises SEQ ID No:1027;
and wherein:
(i) the second spacer comprises SEQ ID NO: 1900, and the second PBS comprises SEQ ID NO: 1908,
(ii) the second spacer comprises SEQ ID NO: 1936, and the second PBS comprises SEQ ID NO: 1944,
or
(iii) the second spacer comprises SEQ ID NO: 2673, and the second PBS comprises SEQ ID NO: 2681.

45. The prime editing composition of any one of claims 29-43, wherein the first spacer comprises SEQ ID No: 77, and the first PBS is 8-10 nucleotides in length and comprises SEQ ID NOs: 87, 86, or 85.

46. The prime editing composition of claim 45, wherein the second spacer comprises SEQ ID NO: 1936, and the second PBS is 8-10 nucleotides in length and comprises SEQ ID NOs: 1946, 1945, or 1944.

47. The prime editing composition of any one of claims 44-46, wherein the first PEgRNA comprises a sequence selected from the group consisting of SEQ ID NOs: 93-102, 552, 553, 1035, and 1036, and wherein the second PEgRNA comprises a sequence selected from the group consisting of SEQ ID NOs: 1952-1961, 1916, 1917, 2689, and 2690.

48. The prime editing composition of any one of claims 44-47, wherein the first PEgRNA comprises a sequence selected from the group consisting of SEQ ID NOs: 94, 552, and 1035; and wherein the second PEgRNA comprises a sequence selected from the group consisting of 1952, 1916, and 2689.

49. The prime editing composition of any one of claims 29-48, wherein the first editing template and the second editing template have the same length and are perfectly complementary to each other.

50. The prime editing composition of any one of claims 1-28, wherein the first editing template comprises at its 3′ end nucleotides 8-17 of the selected sequence for the second spacer, and wherein the second editing template comprises at its 3′ end nucleotides 8-17 of the selected sequence of the first spacer.

51. The prime editing composition of claim 50, wherein the first editing template comprises at its 3′ end nucleotides 1-17, 2-17, 3-17, 4-17, 5-17, 6-17, 7-17, or 8-17 of the selected sequence for the second spacer, and/or wherein the second editing template comprises at its 3′ end nucleotides 1-17, 2-17, 3-17, 4-17, 5-17, 6-17, 7-17, or 8-17 of the selected sequence for the first spacer.

52. The prime editing composition of claim 50, wherein the first editing template comprises at its 3′ end nucleotides 3-17 of the selected sequence for the second spacer, and wherein the second editing template comprises at its 3′ end nucleotides 3-17 of the selected sequence of the first spacer.

53. The prime editing composition of claim 52, wherein the first editing template comprises a region of complementarity to a sequence on the second strand of the DMPK gene that is directly 3′ to nucleotide 3 of the second search target sequence, wherein the region of complementarity is about 20, about 25, about 30 about 35, about 40, about 45, about 50 nucleotides in length.

54. The prime editing composition of claim 53, wherein the region of complementarity is about 20-30 nucleotides in length.

55. The prime editing composition of claim 52, wherein the second editing template comprises a region of complementarity to a sequence on the first strand of the DMPK gene that is directly 3′ to nucleotide 3 of the first search target sequence, wherein the region of complementarity is about 20, about 25, about 30, about 35, about 40, about 45, or about 50 nucleotides in length.

56. The prime editing composition of claim 55, wherein the region of complementarity is about 20-30 nucleotides in length.

57. The prime editing composition of any one of claims 50-56, wherein the first editing template comprises a sequence selected from the group consisting of SEQ ID NOs: 3459-3461, wherein the second spacer comprises at its 3′ end nucleotides 5-20 of SEQ ID NO: 1525, optionally wherein the second spacer comprises at its 3 end SEQ ID NO: 1525.

58. The prime editing composition of any one of claims 50-57, wherein the second editing template comprises a sequence selected from the group consisting of SEQ ID NOs: 3477-3479, wherein the first spacer comprises at its 3′ end nucleotides 5-20 of SEQ ID NO: 484, optionally wherein the first spacer comprises at its 3′ end SEQ ID NO: 484.

59. The prime editing composition of claim 57 or 58, wherein the first PEgRNA comprises a sequence selected from the group consisting of SEQ ID NOs: 503-517, and wherein the second PEgRNA comprises a sequence selected from the group consisting of SEQ ID NOs: 1548-1562.

60. The prime editing composition of any one of claims 1-28, wherein the first editing template comprises from 5′ to 3′ (i) a region of complementarity to the second editing template and (ii) nucleotides 8-17 of the selected sequence for the second spacer; and wherein the second editing template comprises from 5′ to 3′ (i) a region of complementarity to the first editing template and (ii) nucleotides 8-17 of the selected sequence for the first spacer.

61. The prime editing composition of claim 60, wherein the first editing template comprises nucleotides 1-17, 2-17, 3-17, 4-17, 5-17, 6-17, 7-17, or 8-17 of the selected sequence for the second spacer, and/or wherein the second editing template comprises 1-17, 2-17, 3-17, 4-17, 5-17, 6-17, 7-17, or 8-17 of the selected sequence for the first spacer.

62. The prime editing composition of claim 60, wherein the first editing template comprises nucleotides 3-17 of the selected sequence for the second spacer, and wherein the second editing template comprises nucleotides 3-17 of the selected sequence of the first spacer.

63. The prime editing composition of claim 60, wherein the first editing template comprises a region of complementarity to a sequence on the second strand of the DMPK gene that is directly 3′ to nucleotide 3 of the second search target sequence, wherein the region of complementarity is about 20, about 25, about 30, about 35, about 40, about 45, or about 50 nucleotides in length.

64. The prime editing composition of claim 63, wherein the region of complementarity between the first editing template and the sequence on the second strand of the DMPK gene is about 20-30 nucleotides in length.

65. The prime editing composition of claim 60, wherein the second editing template comprises a region of complementarity to a sequence on the first strand of the DMPK gene that is directly 3′ to nucleotide 3 of the first search target sequence, wherein the region of complementarity is about 20, about 25, about 30, about 35, about 40, about 45, or about 50 nucleotides in length.

66. The prime editing composition of claim 65, wherein the region of complementarity between the second editing template and the sequence on the first strand of the DMPK gene is about 20-30 nucleotides in length.

67. The prime editing composition of any one of claims 60-66, wherein the region of complementarity between the first editing template and the second editing template is about 20 to about 80 nucleotides in length.

68. The prime editing composition of claim 67, wherein the region of complementarity between the first editing template and the second editing template is about 20 to about 40 nucleotides in length.

69. The prime editing composition of claim 67, wherein the region of complementarity between the first editing template and the second editing template is 23-38 nucleotides in length.

70. A prime editing composition comprising: a first prime editing guide RNA (PEgRNA) or one or more polynucleotides encoding the first PEgRNA and a second PEgRNA or one or more polynucleotides encoding the second PEgRNA, wherein
(a) the first PEgRNA comprises
(i) a first spacer comprising at its 3′ end nucleotides 5-20 of a Spacer sequence selected from any one of Tables 1A, 2A, 3A, 4A, 5A, 6A, 7A, 8A, 9A, 10A, 11A, 12A, 13A, 14A, 15A, 16A, 17A, 18A, and 19A;
(ii) a first gRNA core capable of binding to a Cas9 protein;
(iii) a first extension arm comprising
a first primer binding site (PBS) comprising a PBS sequence selected from the same Table as the first spacer, and
a first editing template comprising an RTT sequence selected from Table 37 and has an RTT Pairing NO: x in Table 37,
and
(b) the second PEgRNA comprises
(i) a second spacer comprising at its 3′ end nucleotides 5-20 of a Spacer sequence selected from any one of Tables 20A, 21A, 22A, 23A, 24A, 25A, 26A, 27A, 28A, 29A, 30A, 31A, 32A, 33A, 34A, 35A, and 36A;
(ii) a second gRNA core capable of binding to a Cas9 protein;
(iii) a second extension arm comprising
a second primer binding site (PBS) comprising a PBS sequence selected from the same Table as the second spacer, and
a second editing template comprising an RTT sequence selected from Table 38 and has an RTT pairing NO: x in Table 38,
wherein the RTT pairing number x in (a) and (b) are the same integer.

71. The prime editing composition of claim 70, wherein the first editing template and the second editing template have the same length and are perfectly complementary to each other.

72. A prime editing composition comprising a first prime editing guide RNA (PEgRNA) or one or more polynucleotides encoding the first PEgRNA and a second PEgRNA or one or more polynucleotides encoding the second PEgRNA, wherein
(a) the first PEgRNA comprises
(i) a first spacer comprising at its 3′ end nucleotides 5-20 of a Spacer sequence selected from Table xA, wherein x is an integer from 1 to 19;
(ii) a first gRNA core capable of binding to a Cas9 protein;
(iii) a first extension arm comprising
a first primer binding site (PBS) comprising a PBS sequence selected from the same Table as the first spacer, and
a first editing template comprising an RTT sequence selected from Table 39, wherein the Pairing Spacer number of the RTT sequence in Table 39 is y,
and
(b) the second PEgRNA comprises
(iv) a second spacer comprising at its 3′ end nucleotides 5-20 of a Spacer sequence selected from Table yA, wherein y is an integer from 20 to 36;
(v) a second gRNA core capable of binding to a Cas9 protein;
(vi) a second extension arm comprising
a second editing template comprising an RTT sequence selected from Table 40, wherein the Pairing Spacer number of the RTT sequence in Table 40 is x, and
a second primer binding site (PBS) comprising a PBS sequence selected from the same Table as the second spacer,
wherein x in (a) and (b) are the same integer, and wherein y in (a) and (b) are the same integer.

73. The prime editing composition of any one of claims 70-72, wherein the first spacer and/or the second spacer is from 16 to 22 nucleotides in length.

74. The prime editing composition of any one of claims 70-73, wherein the first spacer and/or the second spacer comprises at its 3′ the selected sequence.

75. The prime editing composition of claim 74, wherein the first spacer and/or the second spacer is 20 nucleotides in length.

76. The prime editing composition of any one of claims 70-75, wherein the first gRNA core and the second gRNA core comprise the same sequence.

77. The prime editing composition of claim 76, wherein the first gRNA core and the second gRNA core each comprises SEQ ID NO: 3641.

78. The prime editing composition of any one of claims 70-77, wherein the first spacer, the first gRNA core, the first editing template, and the first PBS form a contiguous sequence in a single molecule.

79. The prime editing composition of claim 78, wherein the first pegRNA comprises from 5′ to 3′ the first spacer, the first gRNA core, the first editing template, and the first PBS.

80. The prime editing composition of any one of claims 70-79, wherein the second spacer, the second gRNA core, the second editing template, and the second PBS form a contiguous sequence in a single molecule.

81. The prime editing composition of claim 80, wherein the second pegRNA comprises from 5′ to 3′ the second spacer, the second gRNA core, the second editing template, and the second PBS.

82. The prime editing composition of any one of claims 1-81, wherein the first PEgRNA and/or the second PEgRNA further comprises 3′ mN*mN*mN*N and 5′mN*mN*mN*modifications, where m indicates that the nucleotide contains a 2′-O-Me modification and a * indicates the presence of a phosphorothioate bond.

83. The prime editing composition of any one of claims 1-82, further comprising a prime editor or one or more polynucleotides encoding the prime editor, wherein the prime editor comprises a Cas9 nickase having a nuclease inactivating mutation in the HNH domain and a reverse transcriptase.

84. The prime editing composition of claim 83, wherein the Cas9 nickase comprises an amino acid sequence having at least 80%, 85%, 90%, 95%, 96%, 97%, 98%, 99%, or 100% identity to SEQ ID NO: 3582.

85. The prime editing composition of claim 83 or 84, wherein the reverse transcriptase comprises an amino acid sequence having at least 80%, 85%, 90%, 95%, 96%, 97%, 98%, 99%, or 100% identity to SEQ ID NO: 3579.

86. The prime editing system of claim 84 or 85, wherein the sequence identities are determined by Needleman-Wunsch alignment of two protein sequences with Gap Costs set to Existence: 11 Extension: 1 where percent identity is calculated by dividing the number of identities by the length of the alignment.

87. The prime editing composition of any one of claims 83-86, wherein the prime editor is a fusion protein.

88. The prime editing composition of any one of claims 83-86, wherein the one or more polynucleotides comprise (a) a first sequence encoding an N-terminal portion of the Cas9 nickase and an intein-N and (b) a second sequence encoding a intein-C, a C-terminal portion of the Cas9 nickase, and the reverse transcriptase.

89. The prime editing composition of any one of claims 83-88, comprising one or more vectors that comprises the one or more polynucleotides encoding the first PEgRNA, the one or more polynucleotides encoding the second PEgRNA, and the one or more polynucleotides encoding the prime editor.

90. The prime editing composition of claim 89, wherein the one or more vectors are AAV vectors.

91. An LNP comprising the prime editing composition of any one of claims 1-90.

92. A method of editing a DMPK gene, the method comprising contacting the DMPK gene with (a) the prime editing composition of any one of claims 1-82 and a prime editor comprising a Cas9 nickase having a nuclease inactivation mutation in the HNH domain and a reverse transcriptase, (b) the prime editing composition of any one of claims 83-90, or (c) the LNP of claim 91.

93. The method of claim 92, wherein the DMPK gene is in a cell.

94. The method of claim 93, wherein the cell is a mammalian cell.

95. The method of claim 94, wherein the cell is a human cell.

96. The method of any one of claims 93-95, wherein the cell is a fibroblast, a myoblast, a myosatellite, a muscle progenitor cell, a cardiomyocyte, a differentiated muscle cell, a skeletal muscle cell, or a smooth muscle cell.

97. The method of any one of claims 93-96, wherein the cell is in a subject.

98. The method of claim 97, wherein the subject is a human.

99. The method of any one of claims 93-98, wherein the cell is from a subject having myotonic dystrophy type 1.

100. A cell generated by the method of any one of claims 92-99.

101. A population of cells generated by the method of any one of claims 92-99.

102. A method for treating myotonic dystrophy type 1 in a subject in need thereof, the method comprising administering to the subject (a) the prime editing composition of any one of claims 1-82 and a prime editor comprising a Cas9 nickase having a nuclease inactivation mutation in the HNH domain and a reverse transcriptase, (b) the prime editing composition of any one of claims 83-90, (c) the LNP of claim 91, (d) the cell of claim 100, or (3) the population of cells of claim 101.
